SEQUENCE LISTING (386 sequences)

SEQ ID NO: 1 - protein, 631 aa, Alicyclobacillus acidocaldarius [sequence omitted]

SEQ ID NO: 2 - protein, 631 aa, synthetic construct
  source 1..631: note = amino acid sequence of SHC/HAC enzyme variant #65
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYSHVFPTLALGRYKQAIERR

SEQ ID NO: 3 - protein, 631 aa, synthetic construct
  source 1..631: note = amino acid sequence of Aac 215G2 SHC/HAC enzyme variant
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 4 - protein, 631 aa, synthetic construct
  source 1..631: note = AacSHC P1
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 5 - protein, 631 aa, synthetic construct
  source 1..631: note = AacSHC P2
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 6 - protein, 631 aa, synthetic construct
  source 1..631: note = AacSHC P3
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 7 - protein, 631 aa, synthetic construct
  source 1..631: note = SHC#65 P1
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYSHVFPTLALGRYKQAIERR

SEQ ID NO: 8 - protein, 631 aa, synthetic construct
  source 1..631: note = SHC#65 P2
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTMFPGDFYLGYTMYSHVFPTLALGRYKQAIERR

SEQ ID NO: 9 - protein, 631 aa, synthetic construct
  source 1..631: note = SHC#65 P3
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTMFPGDFYLGYTMYSHVFPTLALGRYKQAIERR

SEQ ID NO: 10 - protein, 631 aa, synthetic construct
  source 1..631: note = 215G2 P1
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 11 - protein, 631 aa, synthetic construct
  source 1..631: note = 215G2P2
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 12 - protein, 631 aa, synthetic construct
  source 1..631: note = 215G2P3
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 13 - protein, 725 aa, Zymomonas mobilis
  source 1..725: note = amino acid sequence of wild-type ZmoSHC1 AAV90172.1
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQWARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQATNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYMEIDSIGKGPTTPSQTAWALMGLIAANRPEDYEAIAKGCHYLIDRQEQDGSWKEEEFTGTGFPGYGVGQTIKLDDPALSKRLLQGAELSRAFMLRYDFYRQFFPIMALSRAERLIDLNN

SEQ ID NO: 14 - protein, 608 aa, Zymomonas mobilis
  source 1..608: note = amino acid sequence of wild-type ZmoSHC1
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQWARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQATNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYME

SEQ ID NO: 15 - protein, 658 aa, Zymomonas mobilis
  source 1..658: note = amino acid sequence of wild-type ZmoSHC2
MTVSTSSAFHHSPLSDDVEPIIQKATRALLEKQQQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYWARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAVQKAVAWLKTIQNEDGGWGENCDSYALDYSGYEPMDSTASQTAWALLGLMAVGEANSEAVTKGLNWLAQNQDEEGLWKEDYYSGGGFPRVFYLRYHGYSKYFPLWALARYRNLKKANQPIVHYGM

SEQ ID NO: 16 - protein, 684 aa, Bradryhizobium japonicum
  source 1..684: note = amino acid sequence of wild-type BjpSHC
MTVTSSASARATRDPGNYQTALQSTVRAAADWLIANQKPDGHWVGRAESNACMEAQWCLALWFMGLEDHPLRKRLGQSLLDSQRPDGAWQVYFGAPNGDINATVEAYAALRSLGFRDDEPAVRRAREWIEAKGGLRNIRVFTRYWLALIGEWPWEKTPNIPPEVIWFPLWFPFSIYNFAQWARATLMPIAVLSARRPSRPLPPENRLDALFPHGRKAFDYELPVKAGAGGWDRFFRGADKVLHKLQNLGNRLNLGLFRPAATSRVLEWMIRHQDFDGAWGGIQPPWIYGLMALYAEGYPLNHPVLAKGLDALNDPGWRVDVGDATYIQATNSPVWDTILTLLAFDDAGVLGDYPEAVDKAVDWVLQRQVRVPGDWSMKLPHVKPGGWAFEYANNYYPDTDDTAVALIALAPLRHDPKWKAKGIDEAIQLGVDWLIGMQSQGGGWGAFDKDNNQKILTKIPFCDYGEALDPPSVDVTAHIIEAFGKLGISRNHPSMVQALDYIRREQEPSGPWFGRWGVNYVYGTGAVLPALAAIGEDMTQPYIGRACDWLVAHQQADGGWGESCASYMDVSAVGRGTTTASQTAWALMALLAANRPQDKDAIERGCMWLVERQSAGTWDEPEFTGTGFPGYGVGQTIKLNDPALSQRLMQGPELSRAFMLRYGMYRHYFPLMALGRALRPQSHS

SEQ ID NO: 17 - protein, 657 aa, Burkholderia ambifaria
  source 1..657: note = amino acid sequence of wild-type Burkholderia ambifaria SHC
MNDLTEMATLSAGTVPAGLDAAVASATDALLAAQNADGHWVYELEADSTIPAEYVLLVHYLGETPNLELEQKIGRYLRRVQQADGGWPLFTDGAPNISASVKAYFALKVIGDDENAEHMQRARRAIQAMGGAEMSNVFTRIQLALYGAIPWRAVPMMPVEIMLLPQWFPFHLSKVSYWARTVIVPLLVLNAKRPIAKNPRGVRIDELFVDPPVNAGLLPRQGHQSPGWFAFFRVVDHALRAADGLFPNYTRERAIRQAVSFVDERLNGEDGLGAIYPAMANAVMMYDVLGYAEDHPNRAIARKSIEKLLVVQEDEAYCQPCLSPVWDTSLAAHALLETGDARAEEAVIRGLEWLRPLQILDVRGDWISRRPHVRPGGWAFQYANPHYPDVDDTAVVAVAMDRVQKLKHNDAFRDSIARAREWVVGMQSSDGGWGAFEPENTQYYLNNIPFSDHGALLDPPTADVSGRCLSMLAQLGETPLNSEPARRALDYMLKEQEPDGSWYGRWGMNYVYGTWTALCALNAAGLTPDDPRVKRGAQWLLSIQNKDGGWGEDGDSYKLNYRGFEQAPSTASQTAWALLGLMAAGEVNNPAVARGVEYLIAEQKEHGLWDETRFTATGFPRVFYLRYHGYRKFFPLWALARYRNLKRNNATRVTFGL

SEQ ID NO: 18 - protein, 682 aa, Burkholderia ambifaria
  source 1..682: note = amino acid sequence of wild-type Burkholderia ambifaria SHC
MIRRMNKSGPSPWSALDAAIARGRDALMRLQQPDGSWCFELESDATITAEYILMMHFMDKIDDARQEKMARYLRAIQRLDTHGGWDLYVDGDPDVSCSVKAYFALKAAGDSEHAPHMVRARDAILELGGAARSNVFTRILLATFGQVPWRATPFMPIEFVLFPKWVPISMYKVAYWARTTMVPLLVLCSLKARARNPRNIAIPELFVTPPDQERQYFPPARGMRRAFLALDRVVRHVEPLLPKRLRQRAIRHAQAWCAERMNGEDGLGGIFPPIVYSYQMMDVLGYPDDHPLRRDCENALEKLLVTRPDGSMYCQPCLSPVWDTAWSTMALEQARGVAVPEAGAPASALDELDARIARAYDWLAERQVNDLRGDWIENAPADTQPGGWAFQYANPYYPDIDDSAVVTAMLDRRGRTHRNADGSHPYAARVARALDWMRGLQSRNGGFAAFDADCDRLYLNAIPFADHGALLDPPTEDVSGRVLLCFGVTKRADDRASLARAIDYVKRTQQPDGSWWGRWGTNYLYGTWSVLAGLALAGEDPSQPYIARALAWLRARQHADGGWGETNDSYIDPALAGTNAGESTSNCTAWALLAQMAFGDGESESVRRGIAYLQSVQQDDGFWWHRSHNAPGFPRIFYLKYHGYTAYFPLWALARYRRLAGGVSAAGAHAVPASTGADAALA

SEQ ID NO: 19 - protein, 617 aa, Bacillus anthracis
  source 1..617: note = amino acid sequence of wild-type Bacillus anthracis SHC
MLLYEKAHEEIVRRATALQTMQWQDGTWRFCFEGAPLTDCHMIFLLKLLGRDKEIEPFVERVASLQTNEGTWKLHEDEVGGNLSATIQSYAALLASKKYTKEDANMKRAENFIQERGGVARAHFMTKFLLAIHGEYEYPSLFHLPTPIMFLQNDSPFSIFELSSSARIHLIPMMLCLNKRFRVGKKLLPNLNHIAGGGGEWFREDRSPVFQTLLSDVKQIISYPLSLHHKGYEEIERFMKERIDENGTLYSYATASFYMIYALLALGHSLQSSMIQKAIAGITSYIWKMERGNHLQNSPSTVWDTALLSYALQEAQVSKDNKMIQNATAYLLKKQHTKKADWSVHAPALTPGGWGFSDVNTTIPDIDDTTAVLRALARSRGNKNIDNAWKKGGNWIKGLQNNDGGWGAFEKGVTSKLLAKLPIENASDMITDPSTPDITGRVLEFFGTYAQNELPEKQIQRAINWLMNVQEENGSWYGKWGICYLYGTWAVMTGLRSLGIPSSNPSLTRAASWLEHIQHEDGGWGESCHSSVEKRFVTLPFSTPSQTAWALDALISYYDTETPAIRKGVSYLLSNPYVNERYPTGTGLPGAFYIRYHSYAHIYPLLTLAHYIKKYRK

SEQ ID NO: 20 - protein, 720 aa, Frankia alni
  source 1..720: note = amino acid sequence of wild-type Frankia alni SHC
MPAGVGVLVWLDQRLRAMGRPDLVTTTGGAEIPFVLVAATASTVGVALALRRPRHPVGWLFLALGGVLLLSGGTQGYAAYGAVARPGRLPAADLVAIYADAGFIPWLVLVALILHLTPTGRPLSARWGRIALATAVAGGLWLLVGLVTTETMQPPFQSVTNPLLIGGPLGPLLVARRVLGLATGAGVVLAAVSLIVRFRRSVDVERRQLLWVAVAAVPLPVLMAASFAASYAGNNTAAGLAAATLIGLLAIGAGLAIGQYHLYDVEEILSRAVTYLLVSGLLAASYATVVIVVGQSLAGRTGRSQISAVLATLAAVAVTAPAYRKIQEGVDRRFSRRRFETLQVIRRYLRDPDPDVAVEEVLRRALGDPTLAVAYLVDDRRQWVSADGQPANPGNSFMAAVEVYRRGRPIARVTFDRGRAQPGLVRAAATAATAELDNAGLRAAVALQLVEVRQSRTRIAAAQFAERRTIERNLHDGAQQRLLALALQLRAVQLGGDEASLRQAISTGIDQLQAAVVELRELANGLHPAVLADGGLAAALDDVAARTPVPIKISAPDRRYPPDLEAAAWFIACEAMANAVKHAHPTTIAVDVSAPDGQLIVEVRDDGIGGAQPSGPGLRGIADRAEAFGGSLTVHTDPGTGTTIRALLHRRSPLSSGRRSVMIEGCVDVVAVRRFRCRSSRGSGSRRRRSSWRCGGICGSRCRTGMSRSCSRNAASKLIT

SEQ ID NO: 21 - protein, 685 aa, Rhodopseudomonas palustris
  source 1..685: note = amino acid sequence of wild-type Rhodopseudomonas palustris SHC
MDSILAPRADAPRNIDGALRESVQQAADWLVANQKPDGHWVGRAETNATMEAQWCLALWFLGLEDHPLRVRLGRALLDTQRPDGAWHVFYGAPNGDINATVEAYAALRSLGHRDDEEPLRKARDWILSKGGLANIRVFTRYWLALIGEWPWEKTPNILPEVIWLPTWFPFSIYNFAQWARATLMPIAVLSAHRPSRPLAPQDRLDALFPQGRDSFNYDLPARLGAGVWDVIFRKIDTILHRLQDWGARRGPHGIMRRGAIDHVLQWIIRHQDYDGSWGGIQPPWIYGLMALHTEGYAMTHPVMAKALDALNEPGWRIDIGDATFIQATNSPVWDTMLSLLAFDDAGLGERYPEQVERAVRWVLKRQVLVPGDWSVKLPDVKPGGWAFEYANNFYPDTDDTSVALMALAPFRHDPKWQAEGIEDAIQRGIDWLVAMQCKEGGWGAFDKDNDKKILAKIPFCDFGEALDPPSADVTAHIIEAFAKVGLDRNHPSIVRALDYLKREQEPEGPWFGRWGVNYVYGTGAVLPALAAIGEDMRQPYIARACDWLIARQQANGGWGESCVSYMDAKQAGEGTATASQTAWALMALIAADRPQDRDAIERGCLYLTETQRDGTWQEVHYTGTGFPGYGVGQTIKLNDPLLSKRLMQGPELSRSFMLRYDLYRHYFPMMAIGRVLRQRGDRSGH

SEQ ID NO: 22 - protein, 685 aa, Gluconobacter morbifer
  source 1..685: note = amino acid sequence of wild-type GmoSHC
MSPADISTKSSSFQRLDNMLPEAVSSACDWLIDQQKPDGHWVGPVESNACMEAQWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQATNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFILERQEDGSWTEEEYTGTGFPGYGVGQAIKLDDPSLPDRLLQGAELSRAFMLRYDLYRQYFPVMALSRARRMMKEDASAAA

SEQ ID NO: 23 - protein, 642 aa, Thermosynechococcus elongatus
  source 1..642: note = amino acid sequence of wild-type Tel SHC
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSWARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQPCVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAFDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTGFPCHFYLKYHYYQQHFPLTALGRYARWRNLLAT

SEQ ID NO: 24 - protein, 720 aa, Acetobacter pasteurianus
  source 1..720: note = amino acid sequence of wild-type Apa SHC1
MNMASRFSLKKILRSGSDTQGTNVNTLIQSGTSDIVRQKPAPQEPADLSALKAMGNSLTHTLSSACEWLMKQQKPDGHWVGSVGSNASMEAEWCLALWFLGLEDHPLRPRLGKALLEMQRPDGSWGTYYGAGSGDINATVESYAALRSLGYAEDDPAVSKAAAWIISKGGLKNVRVFTRYWLALIGEWPWEKTPNLPPEIIWFPDNFVFSIYNFAQWARATMMPLAILSARRPSRPLRPQDRLDALFPGGRANFDYELPTKEGRDVIADFFRLADKGLHWLQSSFLKRAPSREAAIKYVLEWIIWHQDADGGWGGIQPPWVYGLMALHGEGYQFHHPVMAKALDALNDPGWRHDKGDASWIQATNSPVWDTMLSLMALHDANAEERFTPEMDKALDWLLSRQVRVKGDWSVKLPNTEPGGWAFEYANDRYPDTDDTAVALIAIASCRNRPEWQAKGVEEAIGRGVRWLVAMQSSCGGWGAFDKDNNKSILAKIPFCDFGEALDPPSVDVTAHVLEAFGLLGLPRDLPCIQRGLAYIRKEQDPTGPWFGRWGVNYLYGTGAVLPALAALGEDMTQPYISKACDWLINCQQENGGWGESCASYMEVSSIGHGATTPSQTAWALMGLIAANRPQDYEAIAKGCRYLIDLQEEDGSWNEEEFTGTGFPGYGVGQTIKLDDPAISKRLMQGAELSRAFMLRYDLYRQLFPIIALSRASRLIKLGN

SEQ ID NO: 25 - protein, 625 aa, Bacillus megaterium
  source 1..625: note = amino acid sequence of wild-type BmeSHC
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGLNWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDQSVEKGIKFLLNPNLTEQQTHYPTGIGLPGQFYIQYHSYNDIFPLLALAHYAKKHSS

SEQ ID NO: 26 - protein, 691 aa, Streptomyces albolongus
  source 1..691: note = amino acid sequence of wild-type SalSHC
VTATADGRLDPEYDPEPAAVGERPPVTDRLTGRQTTVATAPAPGAGRRQAEGPERSGPLDPAQALARATAELLSRQSPDGWWKGDLETNVTMDAEDLLLRQFLGIREPEQTAATAAWIRSQQREDGTWSTFYGGPPELSTTVEAYVALKLAGDDPGAPHMAAAARYVRERGGIAASRVFTRIWLALFGWWPWERLPEMPPEIIFLPRWLPLNIYAFGCWARQTIVPLTVVSAHRPVRPAPFDLTELHTDPADPYPLRPLAPPTGWDGVFERLDLVLHAYHKRALRPLRRAALAQAGRWIVERQEADGCWGGIQPPAVYSLIALHLLGYDLEHPVMRAGLAAFDRFTVHTEDGRRWLEACQSPVWDTCLATIALRDAGLPADHPALVSAADWMLAEEIRRPGDWSVQRPRLAPGGWAFEFENDNYPDIDDTAEVVLALKRVAHPDRARIDGAVRRGVEWNLGMQSRNGAWGAFDVDNTSTLPNKLPFCDFGEVVDPPSADVTAHVVEMLAETGLAGDRRTRRGIDWLLKNQEPDGSWFGRWGTNYIYGTGSVLPALVAAGIPGSHPAVRRAVDWLADRQNPDGGWGEDMRSYEDPVRWSGRGDSTASQTAWALMALLAAGEGPDGARSEVVERGVQWLCRTQLPSGSWDEPQFTGTGFPWDFSINYHLYRLVFPVTALGRYLHGSPLTGGGA

SEQ ID NO: 27 - protein, 656 aa, Acetobacter pasteurianus
  source 1..656: note = amino acid sequence of wild-type ApaSHCA
MAADGSALSESRLSSEALDRAVLSAHTALSQAQQDDGHWVYELEADATIPAEYILLEHFMDRIDDALEQKIAIYLRRIQSEEHGGWPLYHNGKFDLSATVKAYFALKAVGDDINAPHMQRAREAILDHGGAERSNVFTRSQLALFGEVPWRATPVMPVELMLLPAKAFFSVWNMSYWSRTVIAPLLVLAALRPVAANPRQVHVRELFVTPPEKVQDWIRGPYRSAWGYVFKGLDSVLRPVVPFIPEKTHKKAIQAALDFIEPRLNGKDGLGAIYPAMANVVMMYRAMGVPDEDPRAKTAWEAVQALIVEKDDEAYCQPCVSPIWDTGLSGHAMIEAASGPNGIAPEKTVAELKKASAWLRSKQILNVKGDWAVRNPNLAPGGWAFQYGNDYYPDVDDTAVVGMLLHREGDPTNAEAIERARTWIVGMQSTDGGWGAFDIDNNKDVLNHIPFADHGALLDPPTADVTARCISFLAQLRNPEDEPVIQRGLEYLRKEQEKDGSWFGRWGTNYIYGTWSALCALNAAGVSHDDPAVVKAVEWLRSVQRADGGWGEGCESYEGGPHGTYGESLPSQTAWAVLGLMAAGRRDDPAVTRGIAWLADQQDANGEWHEDPYNAVGFPKVFYLRYHGYKQFFPLMALARYRNLESSNTRRVSFGF

SEQ ID NO: 28 - protein, 657 aa, Burkholderia ambifaria; identical to SEQ ID NO: 17
  source 1..657: note = strain: bamshc1; note = amino acid sequence of wild-type BamSHC1

SEQ ID NO: 29 - protein, 682 aa, Burkholderia ambifaria; identical to SEQ ID NO: 18
  source 1..682: note = strain: bamshc2; note = amino acid sequence of wild-type BamSHC2

SEQ ID NO: 30 - protein, 695 aa, Pelobacter carbinolicus
  source 1..695: note = strain: pcashc2; note = amino acid sequence of wild-type PcaSHC2
MNVIRQLNSGVNAAKSLDDGIESAIEWLAENQDKEGFWVGMLESNSCIEAEWILAMHLLGVKDDPKYDKVVQAILNEQREDGSWAVYYDAPAGDINATVEAYAALRTAGFGAGDERLIKARNWIFSHGGLKNVRVFTRYWLALIGEWPWDETPALAPEIIYLPAWCPLNIYDFACWARATLVPLSVLSVRRPVKPLPAESRLDELFPEGRENADYSLPESEKGLAERFFLVVDWFLKKYNRLPMQFGREKAIRLCLEWIVRHQDYDGGWGGIQPPLIYSLIALNTEGYGLNHPVISKGLDAFNPPWAYEKNGGVYLQCSESPVWDTLFTMLALFESGCSFDDTPMMRPALDWILSKQITSWGDWQVKVRGVRPGGWAFERANTAYPDVDDTALALVVLAEARRHVKDSAAVDAALERAEEWILGLQCRNGGWAAFDRDNNSAIVTKIPFCDFGEVLDPPSVDVTAHVVEALAALGRDRHDPVVARALKYIRSEQEPGGSWFGRWGVNHIYGTCAVLPALAAIGEDMRAPYVLRAADWLVRHQNDDGGWGESCASYMDDSQCGQGSSTASQTGWALMALVAMSSHDYDEAIRRGLDYLLSHQKSGTWDEPQYTGTGFPGYGVGERTNLKEAGATLDQGCELARGFMINYNMYRHYFPLIAMARARRHLGLAANPRHQDSRSSVEVAPEALRGRACG

SEQ ID NO: 31 - protein, 685 aa, Rhodopseudomonas palustris; identical to SEQ ID NO: 21
  source 1..685: note = strain: rpashc1; note = amino acid sequence of wild-type RpaSHC1 ABJ08391.1

SEQ ID NO: 32 - protein, 680 aa, Streptomyces coelicolor
  source 1..680: note = strain: scoshc; note = amino acid sequence of wild-type ScoSHC
MTATTDGSTGASLRPLAASASDTDITIPAAAAGVPEAAARATRRATDFLLAKQDAEGWWKGDLETNVTMDAEDLLLRQFLGIQDEETTRAAALFIRGEQREDGTWATFYGGPGELSTTIEAYVALRLAGDSPEAPHMARAAEWIRSRGGIASARVFTRIWLALFGWWKWDDLPELPPELIYFPTWVPLNIYDFGCWARQTIVPLTIVSAKRPVRPAPFPLDELHTDPARPNPPRPLAPVASWDGAFQRIDKALHAYRKVAPRRLRRAAMNSAARWIIERQENDGCWGGIQPPAVYSVIALYLLGYDLEHPVMRAGLESLDRFAVWREDGARMIEACQSPVWDTCLATIALADAGVPEDHPQLVKASDWMLGEQIVRPGDWSVKRPGLPPGGWAFEFHNDNYPDIDDTAEVVLALRRVRHHDPERVEKAIGRGVRWNLGMQSKNGAWGAFDVDNTSAFPNRLPFCDFGEVIDPPSADVTAHVVEMLAVEGLAHDPRTRRGIQWLLDAQETDGSWFGRWGVNYVYGTGSVIPALTAAGLPTSHPAIRRAVRWLESVQNEDGGWGEDLRSYRYVREWSGRGASTASQTGWALMALLAAGERDSKAVERGVAWLAATQREDGSWDEPYFTGTGFPWDFSINYNLYRQVFPLTALGRYVHGEPFAKKPRAADAPAEAAPAEVKGS

SEQ ID NO: 33 - protein, 710 aa, Syntrophobacter fumaroxidans
  source 1..710: note = strain: sfushc; note = amino acid sequence of wild-type SfuSHC1
MRRLDTFPPEIPTGSRDKPPSGEEHSCSTPAEPLRSRLDEGILRAVDWLVCDQHPDGFWAGMLQSNSCMEAEWVLAMHFLGIDDDPKYDGVIRAILGEQRADGSWGVFHKAPNGDINTTVECYAALRASGLAPESAPLSSAREWILAGGGLANIRNFTKYWLALIGEWPWEGTPTIPPELIFFPPRMPLNIYHFASWARSTIVPLSILSARRPVRPLPEDRRLDELFPQGRSAFDFRLPRKDGWLSWEGFFHVCDRILRLYARTRRAPFRETAIRVCLEWIIRRQETDGAWSGIQPPWIYALLALHAEGYGLDHPILRAGLRAFDSHWSYERDGGIYLQASESPVWDTVLSLRALADCGEERKASVSIASALEWLLNRQISVPGDWAVRVPSVPCGGWAFQRANSFYPDVDDTAVAIEVLARLRPFTANQSAVDRAIRSARDWVLAMQCSNGGWAAFDRDNDFKLVTKIPFCDFGELLDPPSVDVTAHVIEALAALGWDMTSREIEAAVSFIRREQEAEGSWFGRWGVNHIYGTATVLPALRAIGEDMSSAYVLRAADWLASRQNADGGWGETPASYMDDSLRGVGESTASQTAWAIMGLVAVGSGAHDDTVRRGIDFLLFAQHGGTWEEPQYTGTGFPGYSVGERIRLRDMGASLKQGTELQRAFMINYNLYRHYFPLMALGRARYHLQLRRSAREGGNGETTPNGSAL

SEQ ID NO: 34 - protein, 671 aa, Teredinibacter turnerae
  source 1..671: note = strain: ttushc; note = amino acid sequence of wild-type TtuSHC
MEIQDEVDLLEPQESLTASADSAVDRALFWLLDAQYEDGYWAGILESNACMEAEWLLCFHVLGIANHPMSRGLVQGLLQRQRADGSWDVYYGARAGDINTTVEVYAALRCQGYAADHPDIKRARDWIQLQGGVKQVRVFTRFWLALIGEWPWEETPNLPPEILFFPRWFPFNIYHFAAWARATLVPLCILSARRMVVPLNKKSCLQELFPEDRSAVVALGKKAGAWSTFFYHADRALKKYQRTFKRPPGRQQAIKMCLEWILRRQDADGAWGGIQPPWIYSLMALKAEGYPVTHPVMAKGLAALDAHWSYERPGGARFVQACESPVWDTLLSSFALLDCGFSCTSSSELRKAVDWILDQQVLLPGDWQQKLPTVSPGGWAFERANVHYPDVDDTAVALIVLAKVRPDYPDTARVNLAIERGLNWLFAMQCRNGGWGAFDKDNDKDLLTKIPFSDFGETIDPASVDVTAHVLEALGLLGYRTTHPAVAKALEFIRSEQENDGCWFGRWGVNYIYGTAAVLPALASLNMNMNQEFIRRAANWILGKQNNDGGWGESCASYMDDTQRGRGPSTASQTAWAMMSLLAVDGGTYAESLLRAEAYLKTTQTPEGTWDEPYYTGTGFPGYGIGRREIKRQRSLQQHAELSRGFMINYNLYRHYFPLMALGRLAALRGA

SEQ ID NO: 35 - DNA, 1896 bp, Alicyclobacillus acidocaldarius
  source 1..1896: mol_type = genomic DNA; note = nucleotide sequence encoding the polypeptide of wild-type AacSHC
atggctgagcagttggtggaagcgccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggatgtggctggcgctggtgggagaatatccgtgggagaaggtgcccatggtcccgccggagatcatgttcctcggcaagcgcatgccgctcaacatctacgagtttggctcgtgggctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggcgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacatcccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacggcctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtaccgccacgtgtttccgacgctcgcgctcggccgctacaagcaagccatcgagcgcaggtga

SEQ ID NO: 36 - DNA, 1896 bp, synthetic construct
  source 1..1896: mol_type = other DNA; note = nucleotide sequence of SHC/HAC enzyme variant #65
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgtgggctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacgacctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtacagccacgtgtttccgacgctcgcgctcggccgctacaagcaagccatcgagcgcaggtga

SEQ ID NO: 37 - DNA, 1896 bp, synthetic construct
  source 1..1896: mol_type = other DNA; note = nucleotide sequence encoding Aac 215G2 SHC/HAC enzyme variant
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgtgggctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacggcctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtaccgccacgtgtttccgacgctcgcgctcggccgctacaagcaagccatcgagcgcaggtga

SEQ ID NO: 38 - DNA, 1896 bp, synthetic construct
  source 1..1896: mol_type = other DNA; note = nucleotide sequence encoding the AacSHC P1
atggccgaacagctggttgaagccccggcctatgcccgtaccctggatcgtgccgtggaatatctgctgagttgtcagaaagatgaaggttattggtggggcccgctgctgagcaatgttaccatggaagcagaatatgtgctgctgtgccatattctggatcgcgtggatcgtgatcgtatggaaaaaattcgtcgttatctgctgcatgaacagcgcgaagatggcacctgggcactgtatccgggtggcccgccggatctggataccaccattgaagcctatgtggcactgaaatatattggtatgagccgtgatgaagaaccgatgcagaaagcactgcgcttcattcagagtcagggcggtattgaaagcagtcgtgtgttcacccgcatgtggctggcactggtgggcgaatatccgtgggaaaaagtgccgatggtgccgccggaaattatgttcctgggcaaacgtatgccgctgaatatctatgaattcggcagcggcgcccgcgcaaccgtggtggcactgagcattgtgatgagtcgtcagccggtgttcccgctgccggaacgtgcccgtgtgccggaactgtatgaaaccgatgtgccgccgcgccgtcgcggtgcaaaaggtggtggtggttggatcttcgatgcactggatcgtgcactgcatggttatcagaaactgagtgttcatccgttccgtcgcgccgccgaaattcgcgcactggattggctgctggaacgtcaggcaggcgatggcagttggggtggtattcagccgccgtggttctatgccctgattgccctgaaaattctggatatgacccagcatccggccttcattaaaggctgggaaggtctggaactgtatggcgtggaactggattatggcggttggatgttccaggcaagcattagtccggtgtgggataccggtctggcagtgctggccctgcgcgcagcaggtctgcctgcagatcatgatcgcctggttaaagccggcgaatggctgctggatcgccagattaccgtgccgggtgattgggcagttaaacgtccgaatctgaaaccgggcggcttcgcattccagttcgataatgtgtattatccggatgttgatgataccgccgtggttgtgtgggccctgaataccctgcgcctgccggatgaacgccgccgtcgtgatgccatgaccaaaggcttccgctggattgttggtatgcagagcagcaatggtggctggggcgcatacgatgtggataataccagcgatctgccgaatcatattccgttctgtgacttcggtgaagttaccgatccgccgagcgaagatgttaccgcccatgttctggaatgcttcggtagcttcggttatgatgatgcctggaaagttattcgtcgtgccgttgaatatctgaaacgcgaacagaaaccggatggtagttggttcggtcgctggggtgtgaattatctgtatggcaccggtgcagttgttagtgcactgaaagcagtgggcattgatacccgcgaaccgtatattcagaaagccctggattgggtggaacagcatcagaatccggatggcggttggggtgaagattgtcgtagttatgaagatccggcatacgctggtaaaggtgcaagtaccccgagtcagaccgcatgggccctgatggcactgattgccggtggccgtgcagaaagcgaagcagcccgccgtggcgttcagtatctggtggaaacacaacgcccggatggcggctgggatgaaccgtattataccggcaccggcttcccgggcgacttctatctgggttataccatgtatcgccatgtgttcccgaccctggccctgggccgttataaacaggccattgaacgccgttaa

SEQ ID NO: 39 - DNA, 1896 bp, synthetic construct
  source 1..1896: mol_type = other DNA; note = nucleotide sequence encoding the AacSHC P2
atggccgaacagctggttgaagccccggcctatgcccgtaccctggatcgtgccgtggaatatctgctgagttgtcagaaagatgaaggttattggtggggcccgctgctgagcaatgttaccatggaagcagaatatgtgctgctgtgccatattctggatcgcgtggatcgtgatcgtatggaaaaaattcgtcgttatctgctgcatgaacagcgcgaagatggcacctgggcactgtatccgggtggcccgccggatctggataccaccattgaagcctatgtggcactgaaatatattggtatgagccgtgatgaagaaccgatgcagaaagcactgcgcttcattcagagtcagggcggtattgaaagcagtcgtgtgttcacccgcatgtggctggcactggtgggcgaatatccgtgggaaaaagtgccgatggtgccgccggaaattatgttcctgggcaaacgtatgccgctgaatatctatgaattcggcagcggcgcccgcgcaaccgtggtggcactgagcattgtgatgagtcgtcagccggtgttcccgctgccggaacgtgcccgtgtgccggaactgtatgaaaccgatgtgccgccgcgccgtcgcggtgcaaaaggtggtggtggttggatcttcgatgcactggatcgtgcactgcatggttatcagaaactgagtgttcatccgttccgtcgcgccgccgaaattcgcgcactggattggctgctggaacgtcaggcaggcgatggcagttggggtggtattcagccgccgtggttctatgccctgattgccctgaaaattctggatatgacccagcatccggccttcattaaaggctgggaaggtctggaactgtatggcgtggaactggattatggcggttggatgttccaggcaagcattagtccggtgtgggataccggtctggcagtgctggccctgcgcgcagcaggtctgcctgcagatcatgatcgcctggttaaagccggcgaatggctgctggatcgccagattaccgtgccgggtgattgggcagttaaacgtccgaatctgaaaccgggcggcttcgcattccagttcgataatgtgtattatccggatgttgatgataccgccgtggttgtgtgggccctgaataccctgcgcctgccggatgaacgccgccgtcgtgatgccatgaccaaaggcttccgctggattgttggtatgcagagcagcaatggtggctggggcgcatacgatgtggataataccagcgatctgccgaatcatattccgttctgtgacttcggtgaagttaccgatccgccgagcgaagatgttaccgcccatgttctggaatgcttcggtagcttcggttatgatgatgcctggaaagttattcgtcgtgccgttgaatatctgaaacgcgaacagaaaccggatggtagttggttcggtcgctggggtgtgaattatctgtatggcaccggtgcagttgttagtgcactgaaagcagtgggcattgatacccgcgaaccgtatattcagaaagccctggattgggtggaacagcatcagaatccggatggcggttggggtgaagattgtcgtagttatgaagatccggcatacgctggtaaaggtgcaagtaccccgagtcagaccgcatgggccctgatggcactgattgccggtggccgtgcagaaagcgaagcagcccgccgtggcgttcagtatctggtggaaacacaacgcccggatggcggctgggatgaaccgtattataccggcaccatgttcccgggcgacttctatctgggttataccatgtatcgccatgtgttcccgaccctggccctgggccgttataaacaggccattgaacgccgttaa

SEQ ID NO: 40 - DNA, 1896 bp, synthetic construct
  source 1..1896: mol_type = other DNA; note = nucleotide sequence encoding the AacSHC P3
atggccgaacagctggttgaagccccggcctatgcccgtaccctggatcgtgccgtggaatatctgctgagttgtcagaaagatgaaggttattggtggggcccgctgctgagcaatgttaccatggaagcagaatatgtgctgctgtgccatattctggatcgcgtggatcgtgatcgtatggaaaaaattcgtcgttatctgctgcatgaacagcgcgaagatggcacctgggcactgtatccgggtggcccgccggatctggataccaccattgaagcctatgtggcactgaaatatattggtatgagccgtgatgaagaaccgatgcagaaagcactgcgcttcattcagagtcagggcggtattgaaagcagtcgtgtgttcacccgcatgtggctggcactggtgggcgaatatccgtgggaaaaagtgccgatggtgccgccggaaattatgttcctgggcaaacgtatgccgctgaatatctatgaattcggcagcggcgcccgcgcaaccgtggtggcactgagcattgtgatgagtcgtcagccggtgttcccgctgccggaacgtgcccgtgtgccggaactgtatgaaaccgatgtgccgccgcgccgtcgcggtgcaaaaggtggtggtggttggatcttcgatgcactggatcgtgcactgcatggttatcagaaactgagtgttcatccgttccgtcgcgccgccgaaattcgcgcactggattggctgctggaacgtcaggcaggcgatggcagttggggtggtattcagccgccgtggttctatgccctgattgccctgaaaattctggatatgacccagcatccggccttcattaaaggctgggaaggtctggaactgtatggcgtggaactggattatggcggttggatgttccaggttagcattagtccggtgtgggataccggtctggcagtgctggccctgcgcgcagcaggtctgcctgcagatcatgatcgcctggttaaagccggcgaatggctgctggatcgccagattaccgtgccgggtgattgggcagttaaacgtccgaatctgaaaccgggcggcttcgcattccagttcgataatgtgtattatccggatgttgatgataccgccgtggttgtgtgggccctgaataccctgcgcctgccggatgaacgccgccgtcgtgatgccatgaccaaaggcttccgctggattgttggtatgcagagcagcaatggtggctggggcgcatacgatgtggataataccagcgatctgccgaatcatattccgttctgtgacttcggtgaagttaccgatccgccgagcgaagatgttaccgcccatgttctggaatgcttcggtagcttcggttatgatgatgcctggaaagttattcgtcgtgccgttgaatatctgaaacgcgaacagaaaccggatggtagttggttcggtcgctggggtgtgaattatctgtatggcaccggtgcagttgttagtgcactgaaagcagtgggcattgatacccgcgaaccgtatattcagaaagccctggattgggtggaacagcatcagaatccggatggcggttggggtgaagattgtcgtagttatgaagatccggcatacgctggtaaaggtgcaagtaccccgagtcagaccgcatgggccctgatggcactgattgccggtggccgtgcagaaagcgaagcagcccgccgtggcgttcagtatctggtggaaacacaacgcccggatggcggctgggatgaaccgtattataccggcaccatgttcccgggcgacttctatctgggttataccatgtatcgccatgtgttcccgaccctggccctgggccgttataaacaggccattgaacgccgttaa

SEQ ID NO: 41 - DNA, 1897 bp, synthetic construct
  source 1..1897: mol_type = other DNA; note = nucleotide sequence encoding the SHC#65P1
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgggcgctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacgacctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtacagccacgtgtttccgacgctcgcgctcggccgctacaagcaagccatcgagcgcaggtgaa

SEQ ID NO: 42 - DNA, 1897 bp, synthetic construct
  source 1..1897: mol_type = other DNA; note = nucleotide sequence encoding the SHC#65P2
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgggcgctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacgacctgggcgctgatggcgctcatcgcgggcggcagggcggagtccgaggccgcgcgccgcggcgtgcaatacctcgtggagacgcagcgcccggacggcggctgggatgagccgtactacaccggcacgatgttcccaggggatttctacctcggctacaccatgtacagccacgtgtttccgacgctcgcgctcggccgctacaagcaagccatcgagcgcaggtgaa

SEQ ID NO: 43 - DNA, 1897 bp, synthetic construct
  source 1..1897: mol_type = other DNA; note = nucleotide sequence encoding the SHC#65 P3
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgggcgctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggtttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacgacctgggcgctgatggcgctcatcgcgggcggcagggcggagtccgaggccgcgcgccgcggcgtgcaatacctcgtggagacgcagcgcccggacggcggctgggatgagccgtactacaccggcacgatgttcccaggggatttctacctcggctacaccatgtacagccacgtgtttccgacgctcgcgctcggccgctacaagcaagccatcgagcgcaggtgaa

SEQ ID NO: 44 - DNA, 1896 bp, synthetic construct
  source 1..1896: mol_type = other DNA; note = nucleotide sequence encoding the 215G2P1
atggccgaacagctggttgaagccccggcctatgcccgtaccctggatcgtgccgtggaatatctgctgagttgtcagaaagatgaaggttattggtggggcccgctgctgagcaatgttaccatggaagcagaatatgtgctgctgtgccatattctggatcgcgtggatcgtgatcgtatggaaaaaattcgtcgttatctgctgcatgaacagcgcgaagatggcacctgggcactgtatccgggtggcccgccggatctggataccaccattgaagcctatgtggcactgaaatatattggtatgagccgtgatgaagaaccgatgcagaaagcactgcgcttcattcagagtcagggcggtattgaaagcagtcgtgtgttcacccgccgttggctggcactggtgggcgaatatccgtgggaaaaagtgccgatggtgccgccggaaattatgttcctgggcaaacgtatgccgctgaatatctatgaattcggcagcggcgcccgcgcaaccgtggtggcactgagcattgtgatgagtcgtcagccggtgttcccgctgccggaacgtgcccgtgtgccggaactgtatgaaaccgatgtgccgccgcgccgtcgcggtgcaaaaggtggtggtggttggatcttcgatgcactggatcgtgttctgcatggttatcagaaactgagtgttcatccgttccgtcgcgccgccgaaattcgcgcactggattggctgctggaacgtcaggcaggcgatggcagttggggtggtattcagccgccgtggttctatgccctgattgccctgaaaattctggatatgacccagcatccggccttcattaaaggctgggaaggtctggaactgtatggcgtggaactggattatggcggttggatgttccaggcaagcattagtccggtgtgggataccggtctggcagtgctggccctgcgcgcagcaggtctgcctgcagatcatgatcgcctggttaaagccggcgaatggctgctggatcgccagattaccgtgccgggtgattgggcagttaaacgtccgaatctgaaaccgggcggcttcgcattccagttcgataatgtgtattatccggatgttgatgataccgccgtggttgtgtgggccctgaataccctgcgcctgccggatgaacgccgccgtcgtgatgccatgaccaaaggcttccgctggattgttggtatgcagagcagcaatggtggctggggcgcatacgatgtggataataccagcgatctgccgaatcataccccgttctgtgacttcggtgaagttaccgatccgccgagcgaagatgttaccgcccatgttctggaatgcttcggtagcttcggttatgatgatgcctggaaagttattcgtcgtgccgttgaatatctgaaacgcgaacagaaaccggatggtagttggttcggtcgctggggtgtgaattatctgtatggcaccggtgcagttgttagtgcactgaaagcagtgggcattgatacccgcgaaccgtatattcagaaagccctggattgggtggaacagcatcagaatccggatggcggttggggtgaagattgtcgtagttatgaagatccggcatacgctggtaaaggtgcaagtaccccgagtcagaccgcatgggccctgatggcactgattgccggtggccgtgcagaaagcgaagcagcccgccgtggcgttcagtatctggtggaaacacaacgcccggatggcggctgggatgaaccgtattataccggcaccggcttcccgggcgacttctatctgggttataccatgtatcgccatgtgttcccgaccctggccctgggccgttataaacaggccattgaacgccgttaa

SEQ ID NO: 45 - DNA, 1896 bp, synthetic construct
  source 1..1896: mol_type = other DNA; note = nucleotide sequence encoding the215G2 P2
atggccgaacagctggttgaagccccggcctatgcccgtaccctggatcgtgccgtggaatatctgctgagttgtcagaaagatgaaggttattggtggggcccgctgctgagcaatgttaccatggaagcagaatatgtgctgctgtgccatattctggatcgcgtggatcgtgatcgtatggaaaaaattcgtcgttatctgctgcatgaacagcgcgaagatggcacctgggcactgtatccgggtggcccgccggatctggataccaccattgaagcctatgtggcactgaaatatattggtatgagccgtgatgaagaaccgatgcagaaagcactgcgcttcattcagagtcagggcggtattgaaagcagtcgtgtgttcacccgccgttggctggcactggtgggcgaatatccgtgggaaaaagtgccgatggtgccgccggaaattatgttcctgggcaaacgtatgccgctgaatatctatgaattcggcagcggcgcccgcgcaaccgtggtggcactgagcattgtgatgagtcgtcagccggtgttcccgctgccggaacgtgcccgtgtgccggaactgtatgaaaccgatgtgccgccgcgccgtcgcggtgcaaaaggtggtggtggttggatcttcgatgcactggatcgtgttctgcatggttatcagaaactgagtgttcatccgttccgtcgcgccgccgaaattcgcgcactggattggctgctggaacgtcaggcaggcgatggcagttggggtggtattcagccgccgtggttctatgccctgattgccctgaaaattctggatatgacccagcatccggccttcattaaaggctgggaaggtctggaactgtatggcgtggaactggattatggcggttggatgttccaggcaagcattagtccggtgtgggataccggtctggcagtgctggccctgcgcgcagcaggtctgcctgcagatcatgatcgcctggttaaagccggcgaatggctgctggatcgccagattaccgtgccgggtgattgggcagttaaacgtccgaatctgaaaccgggcggcttcgcattccagttcgataatgtgtattatccggatgttgatgataccgccgtggttgtgtgggccctgaataccctgcgcctgccggatgaacgccgccgtcgtgatgccatgaccaaaggcttccgctggattgttggtatgcagagcagcaatggtggctggggcgcatacgatgtggataataccagcgatctgccgaatcataccccgttctgtgacttcggtgaagttaccgatccgccgagcgaagatgttaccgcccatgttctggaatgcttcggtagcttcggttatgatgatgcctggaaagttattcgtcgtgccgttgaatatctgaaacgcgaacagaaaccggatggtagttggttcggtcgctggggtgtgaattatctgtatggcaccggtgcagttgttagtgcactgaaagcagtgggcattgatacccgcgaaccgtatattcagaaagccctggattgggtggaacagcatcagaatccggatggcggttggggtgaagattgtcgtagttatgaagatccggcatacgctggtaaaggtgcaagtaccccgagtcagaccgcatgggccctgatggcactgattgccggtggccgtgcagaaagcgaagcagcccgccgtggcgttcagtatctggtggaaacacaacgcccggatggcggctgggatgaaccgtattataccggcaccatgttcccgggcgacttctatctgggttataccatgtatcgccatgtgttcccgaccctggccctgggccgttataaacaggccattgaacgccgttaa

SEQ ID NO: 46 - DNA, 1896 bp, synthetic construct
  source 1..1896: mol_type = other DNA; note = nucleotide sequence encoding the 215G2 P3
atggccgaacagctggttgaagccccggcctatgcccgtaccctggatcgtgccgtggaatatctgctgagttgtcagaaagatgaaggttattggtggggcccgctgctgagcaatgttaccatggaagcagaatatgtgctgctgtgccatattctggatcgcgtggatcgtgatcgtatggaaaaaattcgtcgttatctgctgcatgaacagcgcgaagatggcacctgggcactgtatccgggtggcccgccggatctggataccaccattgaagcctatgtggcactgaaatatattggtatgagccgtgatgaagaaccgatgcagaaagcactgcgcttcattcagagtcagggcggtattgaaagcagtcgtgtgttcacccgccgttggctggcactggtgggcgaatatccgtgggaaaaagtgccgatggtgccgccggaaattatgttcctgggcaaacgtatgccgctgaatatctatgaattcggcagcggcgcccgcgcaaccgtggtggcactgagcattgtgatgagtcgtcagccggtgttcccgctgccggaacgtgcccgtgtgccggaactgtatgaaaccgatgtgccgccgcgccgtcgcggtgcaaaaggtggtggtggttggatcttcgatgcactggatcgtgttctgcatggttatcagaaactgagtgttcatccgttccgtcgcgccgccgaaattcgcgcactggattggctgctggaacgtcaggcaggcgatggcagttggggtggtattcagccgccgtggttctatgccctgattgccctgaaaattctggatatgacccagcatccggccttcattaaaggctgggaaggtctggaactgtatggcgtggaactggattatggcggttggatgttccaggttagcattagtccggtgtgggataccggtctggcagtgctggccctgcgcgcagcaggtctgcctgcagatcatgatcgcctggttaaagccggcgaatggctgctggatcgccagattaccgtgccgggtgattgggcagttaaacgtccgaatctgaaaccgggcggcttcgcattccagttcgataatgtgtattatccggatgttgatgataccgccgtggttgtgtgggccctgaataccctgcgcctgccggatgaacgccgccgtcgtgatgccatgaccaaaggcttccgctggattgttggtatgcagagcagcaatggtggctggggcgcatacgatgtggataataccagcgatctgccgaatcataccccgttctgtgacttcggtgaagttaccgatccgccgagcgaagatgttaccgcccatgttctggaatgcttcggtagcttcggttatgatgatgcctggaaagttattcgtcgtgccgttgaatatctgaaacgcgaacagaaaccggatggtagttggttcggtcgctggggtgtgaattatctgtatggcaccggtgcagttgttagtgcactgaaagcagtgggcattgatacccgcgaaccgtatattcagaaagccctggattgggtggaacagcatcagaatccggatggcggttggggtgaagattgtcgtagttatgaagatccggcatacgctggtaaaggtgcaagtaccccgagtcagaccgcatgggccctgatggcactgattgccggtggccgtgcagaaagcgaagcagcccgccgtggcgttcagtatctggtggaaacacaacgcccggatggcggctgggatgaaccgtattataccggcaccatgttcccgggcgacttctatctgggttataccatgtatcgccatgtgttcccgaccctggccctgggccgttataaacaggccattgaacgccgttaa

SEQ ID NO: 47 - protein, 631 aa, synthetic construct
  source 1..631: note = amino acid sequence of SHC/HAC enzyme variant #66
MAEQLVEAPAYARTLDRAVEYLLSCQKDEGYWWGPLLSNVTMEAEYVLLCHILDRVDRDRMEKIRRYLLHEQREDGTWALHPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYSHVFPTLALGRYKQAIERR

SEQ ID NO: 48 - protein, 631 aa, synthetic construct
  source 1..631: note = amino acid sequence of SHC/HAC enzyme variant #110B8
MAEQLVEAPAYARTLDRAVEYLLSCQKDEGYWWGPLLSNVTMEAEYVLLCHILDRVDRDRMEKIRRYLLHEQREDGTWALHPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNLTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYRHVFPTLALGRYKQAIERR

SEQ ID NO: 49 - protein, 631 aa, synthetic construct
  source 1..631: note = amino acid sequence of SHC/HAC enzyme variant #90C7
MAEQLVEAPAYARTLDRAVEYLLSCQKDEGYWWGPLLSNVTMEAEYVLLCHILDRVDRDRMEKIRRYLLHEQREDGTWALYPGGPPDLDATIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYSHVFPTLALGRYKQAIERR

SEQ ID NO: 50 - protein, 631 aa, synthetic construct
  source 1..631: note = amino acid sequence of SHC/HAC enzyme variant #115A7
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARTTVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDKTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 51 - protein, 631 aa, synthetic construct
  source 1..631: note = amino acid sequence of SHC/HAC variant 215G2 + L37Q
MAEQLVEAPAYARTLDRAVEYLLSCQKDEGYWWGPLQSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 52 - protein, 631 aa, synthetic construct
  source 1..631: note = amino acid sequence of SHC/HAC variant 215G2 + V174I
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATIVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 53 - protein, 631 aa, synthetic construct
  source 1..631: note = amino acid sequence of SHC/HAC variant 215G2 + V174I + F601Y
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATIVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGYPGDFYLGYTMYRHVFPTLALGRYKQAIERR

SEQ ID NO: 54 - protein, 631 aa, synthetic construct
  source 1..631: note = amino acid sequence of SHC/HAC variant 215G2 + L37Q + V174I + F601Y
MAEQLVEAPAYARTLDRAVEYLLSCQKDEGYWWGPLQSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATIVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGYPGDFYLGYTMYRHVFPTLALGRYKQAIERR

SEQ ID NO: 55 - protein, 720 aa, Acetobacter pasteurianus; identical to SEQ ID NO: 24
  source 1..720: note = strain: ifo 3283-01; note = BAH99456.1 squalene--hopene cyclase [Acetobacter pasteurianus IFO 3283-01]

SEQ ID NO: 56 - protein, 648 aa, Acidithiobacillus caldus
  source 1..648: note = strain: atcc 51756; note = EET25937.1 Squalene--hopene cyclase [Acidithiobacillus caldus ATCC 51756]
MNPVVHNLTRPHRSAEPRPSALQRSIAAAQAALLQHQAADGHWCFEFEADCTIPAEYILMMHYMDERDAALEAKMAAYLRRKQENHGGWSLYHGGHFDMSASVKAYFALKLAGDDPEAAHMRRARSAILAHGGAERANVFTRITLALFGQVPWRAVPFIPVEILLFPRWFPMHIYKVASWSRTVMVPLFILCSLKPQAKNPLGVHIRELFTRPPEDIDDYFAHALQGWVSRIFLWFDRLGRALESWIPQALRRRAIARAEAWFIERLNGEDGLNGIFPAMVNAHEALALLGYAAEHPYRQQTRAALTKLVVERAGEAYCQPCVSPVWDTCLALHALLEADGDVSEAARRSMQWLLDRQITDAPGDWRERRPHLAGGGWAFQYANPYYPDLDDTAAVAWALARARRPEDRPAVERAANWLAGMQSRNGGFGAYDVDNTYYYLNEIPFADHKALLDPPTADVSGRVLAFLAILDREQDAPVRARLIQYLLREQEPSGAWFGRWGTNYIYGTWSVLMGMAELRDPGAEVRDAMARAAHWLRSVQQDDGGWGESNDSYADPGLAGLGQESTAAQTAWACLALMAAGDSDSESLRRGIQWLQRHQEQPGDWQDPYFNAPGFPRVFYLTYHGYKIYFPLWALARYRNITERHCA

SEQ ID NO: 57 - protein, 643 aa, Acidithiobacillus ferrooxidans
  source 1..643: note = strain: atcc 53993; note = ACH84004.1 squalene-hopene cyclase [Acidithiobacillus ferrooxidans ATCC 53993]
MNRMLQPLHSGAGIFRSSLDRVIAQARQALGGRQAEDGHWCFEFEADCTIPAEYILMQHYMDERDEALEARIAVYLRGKQADHGGWPLYYGGHFDLSASVKVYYALKLAGDDPELPHMRRAREAILAHGGAERSNVFTRITLALFAQVPWRAVPFIPVEIMLLPRWFPFHIYKVASWSRTVMVPLFILCSLKARAKNPLQVHIRELFRRPPDQITDYFSHARRGIVAYIFLSLDRFWRLMEGWIPHGIRRRALKKAEAWFTARINGEDGLNGIFPAMVNAHEALELLGYPPDHDYRRQTGAALRKLVVERANDAYCQPCVSPVWDTCLALHALLEEDGEVSPAVQNGIRWLKNRQIGAEPGDWRESRPHLAGGGWAFQYANPYYPDLDDTAAVGWALARAGRAEDRDSIEKAANWLAGMQSRNGGFGAYDVDNTHYYLNEIPFADHKALLDPPTADVTGRVVAFLAHLARPRDRDVLRRAVAYLLREQESSGAWFGRWGTNYIYGTWSVLMALAELNDPSLKPTMERAAYWLRAVQQGDGGWGESNDSYSDPGLAGMGQTSTAAQTAWACLGLMAAGDRDSVALHRGIAWLQAHQEGDGCWQAPFFNAPGFPKVFYLIYHGYAFYFPLWALARYRNLGCMAHE

SEQ ID NO: 58 - protein, 633 aa, Acidothermus cellulolyticus
  source 1..633: note = strain: 11b; note = ABK53469.1 squalene-hopene cyclase [Acidothermus cellulolyticus 11B]
MTQASVREDAKAALDRAVDYLLSLQDEKGFWKGELETNVTIEAEDLLLREFLGIRTPDITAETARWIRAKQRSDGTWATFYDGPPDLSTSVEAYVALKLAGDDPAAPHMEKAAAYIRGAGGVERTRVFTRLWLALFGLWPWDDLPTLPPEMIFLPSWFPLNIYDWGCWARQTVVPLTIVSALRPVRPIPLSIDEIRTGAPPPPRDPAWTIRGFFQRLDDLLRGYRRVADHGPARLFRRLAMRRAAEWIIARQEADGSWGGIQPPWVYSLIALHLLGYPLDHPVLRRGLDGLNGFTIREETADGAVRRLEACQSPVWDTALAVTALRDAGLPADHPRVQAAARWLVGEEVRVAGDWAVRRPGLPPGGWAFEFANDNYPDTDDTAEVVLALRRVRLEDADQQALEAAVRRATTWVIGMQSTDGGWGAFDADNTRELVLRLPFCDFGAVIDPPSADVTAHIVEMLAALGMRDHPATVAGVRWLLAHQEPDGSWFGRWGANHIYGTGAVVPALIAAGVSPDTPPIRRAIRWLEEHQNPDGGWGEDLRSYTDPALWVGRGVSTASQTAWALLALLAAGEEASPAVDRGVRWLVTTQQPDGGWDEPHYTGTGFPGDFYINYHLYRLVFPISALGRYVNR

SEQ ID NO: 59 - protein, 695 aa, Adiantum capillus-veneris
  source 1..695: note = BAF93209.1 hydroxyhopane synthase [Adiantum capillus-veneris]
MLPYNQDHHFGKVAENATMPPTLDEAIERSQDFLLSLQYPEGYWWAELEANVTLTAQTIMLYKILGIDHKYPIHKMKTYILRTQRAHGGWEIFYGDGGCLSTTIGAYMALRILGVPKTDPVLQKALKLIHSKGGVTKSRMFTKICLALLGCYDWKGIPSLPPWLVLLPSWFPFSLYDTASWVRGCVVPLTIIFDKKPVYKLNPLLCLDELYSEGKGKARVHLSFIPGDWTSNFFVGLDHVFKYMENLGVVPFRQWGIKEAERWTLERHEDSGDFHGIYPPMFYSIVSYSLLGYEITDPVVHRALESMRGFTVEREDECVVQSCISPMWDTAFVIRSLAESGLQPDHPALQKAGEWLLQKQATQHGNWFYKKRTGRAGGWAFQFFNRWYPDVDDSAAVSMALNAIKLQDDDVKKGAIKRCAEWISVMQCKDGGWAAYDCDNDREWLNCTPFGDLKAMIDPNTVDVTARVLEMVGRVKEAGDASAILPPRAIARGLAYLRREQETEGCWYGRWGVNYIYGTSGALMALALVAPSTHKEEIERGARWLVEVQNKRGTKGANGYSHTNGAREGGVAMNGNCKNMGAPEDGGWGETCFSYNDITLKGRNEVSTVSQTAWALQGLLAAGDALGKYEVESIEHGVQYLLSTQRKDGSWCEKHFTGGGFPRFFYIRYHLYAGHFPLSALARYRDRVRAGKMAK

SEQ ID NO: 60 - protein, 648 aa, Histoplasma capsulatum
  source 1..648: note = strain: h143; note = EER40510.1 squalene-hopene cyclase [Histoplasma capsulatum H143]
MAQMASSLGSPRLLLRMGREAAQQQHLASGTEVQKALRLAVGHSLDLQRTDGAWCGEVHSNATFTAQYVFLQQQIGLPLDPTEIEGLSRWLFSQQNEDGSWGLGPGLGGDVSTTTETYLALKILGVSPEDPRMAAARTSIIKAGSLPATRMFTRVFLASFGLIPWSAVPPLPAELILLPTLFPVNIYNLSSWARATCVPLLLIRHHEPLHSLPNGRHAENDFLDELWTKDIPRDFCYTTPLSRMWRLGDYAGIFFTSADHGFRFLGQYFNSPLRNLSRRKIINWILDHQEQSGEWAGYWPPQHNNIWALSLEGYSLDHPVLRRGIAAVKSFVLHDATGMRAQVTVSQVWDTALMSIALSDSAPSTGIISPTQAIDWLMHHEVASHRGDWRVLRPKLATGGFCFEEFNTLYPDVDDTAAVIMALIKSNPAHLISGCVRQCFGMMMAGRHGYSLDCQLETRLRASSQLAIAYLLGCQENNGSWWGRWGVNYLYGTSNVLCGLAYYYDRSSLSKGDGKSNSNIVSAVDRASEWLKARQHSNGGWGEGLESYDNAQLAGCGQPTASQSAWVTMALLNYLSPTDEVIQRGVSYLVRNQVKYGDESRATWPLERYTATGFPGHLYMEYDYYRHYFPIMALGRYVNKLSGSHKLL

SEQ ID NO: 61 - protein, 673 aa, Histoplasma capsulatum
  source 1..673: note = strain: g186ar; note = EEH02950.1 squalene-hopene cyclase [Histoplasma capsulatum G186AR]
MLLAEVQKALRLAVGHSLDLQRADGAWCGEVHSNATFTSQYVFLQQQIGLPLDPTEIEGLSRWLFSQQNEDGSWGLGPGLGGDVSTTTETYLALKILGVSPEDPRMAAARTSIIKAGSLPATRMFTRVFLASFGLIPWSAVPPLPAELILLPTLFPVNIYNLSSWARATCVPLLLIRHHEPLHSLPNGRHAENDFLDELWTKDIPRDFCYTTPLSRMWRLGDYAGIFFTSADHGLRFLGQYFHSPLRNLSRRKIINWILDHQEQSGEWAGYWPPQHNNIWALSLEGYSLDHPVLRRGIAAVKSFVLHDATGMRAQVTVSQVWDTALMSIALSDSAPSTGIISPTQAIDWLMHHEVASHRGDWRVLRPKLATGGFCFEEFNTLYPDVDDTAAVIMALIKSNPAHLISGCVRRAAQWILGMQNRDGGWGAFDWNNDKFFLNKIPFSDMDSLCDPSTPDVTGRIIECFGMMMAGRHGYSLDCQLENRLRASSQLAIAYLLGCQENNGSWWGRWGVNYLYGTSNVLCGLAYYYDRSSLSKGDVKSNSNIVSAVDRASEWLKARQHSNGGWGEGPESYDNAQLAGCGQPTASQSAWVTMALLNYLSPTDEVIQRGVSYLVRNQVKYGDESRATWLLERYTATGFPGHLYMEYDYYRHYFPIMALGRYVNKLSGSHKLL

SEQ ID NO: 62 - protein, 631 aa, Alicyclobacillus acidocaldarius
  source 1..631: note = strain: laa1; note = EED08231.1 squalene/oxidosqualene cyclase [Alicyclobacillus acidocaldarius LAA1]
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVAISIVMSRQPVFPLPERARVPELYDTDVPPRRRGAKGGGGRIFDALDRALHGYQKLSVHPFRRAAEIRALDWLLERQAGDGSWGGIQPPWFYTLIALKILDMTQHPAFIKGWEGLELYGVDLDYGGWMFQASISPVWDTGLAVLALRAAGLPADHDRLVKAGEWLLDRQITVPGDWAVKRPNLKPGGFAFQFDNVYYPDVDDTAVVVWALNSLRLPDERRRRDVMTKGFRWIVGMQSSNGGWGAYDVDNTSDLPNHIPFCDFGEVTDPPSEDVTAHVLECFGSFGYDDAWKVIRRAVEYLKREQRPDGSWFGRWGVNYLYGTGAVVPALKAVGIDVREPFIQKALDWVEQHQNPDGGWGEDCRSYEDPAYAGKGASTPSQTAWALMALIAGGRAESDSVRRGVQYLVETQRPDGGWDEPYYTGTGFPGDFYLGYTMYRHVFPTLALGRYKQAIERR

SEQ ID NO: 63 - protein, 227 aa, Alicyclobacillus acidocaldarius
  source 1..227: note = AAT70690.1 squalene-hopene cyclase - partial [Alicyclobacillus acidocaldarius]
QASISPVWDTGLAVLALRAAGLPADHDRLVKAGEWLLDRQITVPGDWVVKRPNLNPGGFALQFDNVYYPDVDDTAVVIWALNTLRLPDERRRRDAMTKGFRWIVGMQSSNGGWGAYDVDNTSDLPNHIPFCDFGEVTDPPSEDVTAHVLECFGSFGYDDAWKVIQRAVAYLKREQKPDGSWFGRWGVNYIYGTGAVVSALKAVGIDMREPYIQKALDWVEQHQNPDG

SEQ ID NO: 64 - protein, 634 aa, Alicyclobacillus acidoterrestris
  source 1..634: note = CAA61950.1 squalene-hopene cyclase [Alicyclobacillus acidoterrestris]
MTKQLLDTPMVQATLEAGVAHLLRRQAPDGYWWAPLLSNVCMEAEYVLLCHCLGKKNPEREAQIRKYIISQRREDGTWSIYPGGPSDLNATVEAYVALKYLGEPASDPQMVQAKEFIQNEGGIESTRVFTRLWLAMVGQYPWDKLPVIPPEIMHLPKSVPLNIYDFASWARATIVTLSYRHESPTCDATSGLCKGSGIVRGEGPPKRRSAKGGDSGFFVALDKFLKAYNKWPIQPGRKSGEQKALEWILAHQEADGCWGGIQPPWFYALLALKCLNMTDHPAFVKGFEGLEAYGVHTSDGGWMFQASISPIWDTGLTVLALRSAGLPPDHPALIKAGEWLVSKQILKDGDWKVRRRKAKPGGWAFEFHCENYPDVDDTAMVVLALNGIQLPDEGKRRDALTRGFRWLREMQSSNGGWGAYDVDNTRQLTKSDSIFATSGEVIDPPSEDVTAHVLECFGSFGYDEAWKVIRKAVEYLKAQQRPDGSWFGRWGVNYVYGIGAVVPGLKAVGVDMREPWVQKSLDWLVEHQNEDGGWGEDCRSYDDPRLAGQGVSTPSQTAWALMALIAGGRVESDAVLRGVTYLHDTQRADGGWDEEVYTGTGFPGDFYLAYTMYRDILPVWALGRYQEAMQRIRG

SEQ ID NO: 65 - protein, 637 aa, Trichormus variabilis
  source 1..637: note = strain: atcc 29413; note = ABA24268.1 Terpene synthase [Trichormus variabilis ATCC 29413]
MRTQDRVQVNSIAEAIAASQKYLLSLQNPTGYWWAELESNVTITAEVVLLHKIWGTDKTRPLHKIEAYLRSQQKQHGGWELFYGDGGELSTSVEAYMALKLLGVPATDPAMIQARDFILQRGGISKTRIFTKFHLALIGCYNWRGLPSLPAWVMLLPNQFPVNIYEMSSWARSSTVPLLIVFDQKPVYQVNPAITLDELYAEGVENVRYELPRSGDWTDLFLTLDEGFKLAESFNFIPFREEGIKAAEKWIIERQEATGDWGGIIPAMLNSMLALRVLGYATNDPIVERGLQAIDNFAIETADCYRVQPCVSPVWDTAWVIRALIDSGMAPDHPAIVKAGEWLLQKQIFDYGDWNVKNRQGQPGAWAFEFDNRFYPDVDDTAVVVMALHAAKLPHEQLKQKACDRALQWVASMQCKPGGWAAFDIDNDQDWLNAVPYGDLKAMIDPNTADVTARVIEMLGACNLSIDSHDLERALTYLLNEQEAEGCWFGRWGVNYIYGTSGVLCALALINPQKYQRHIQQGATWLVGCQNPDGGWGETCFSYNDPSLKGQGDSTPSQTAWALIGLIAAGEATGNFAHDVIERGINHLVSTQQPDGSWFEAYFTGTGFPCHFYLKYHYYQQYFPLIALGRYQAINPL

SEQ ID NO: 66 - protein, 654 aa, Anaeromyxobacter sp.
  source 1..654: note = strain: fw109-5; note = ABS28257.1 squalene-hopene cyclase [Anaeromyxobacter sp. Fw109-5]
MSGEVRVAGDALAEDAGRAAAAASQYLYRTQQRDHWRAELESNVTVTAEYVLLRQALGLDLEERRDALVRYLCSRQKADGSFGIASTLPGDVSTTAEAYLALRLLGLDREDERLRAAERFIRGAGGLARVRVFTRINLALFGLFPWEAVPTVPAELIFLPRWAPVNVYRLASWARSTMVPLFVLFHHRPVFALPGGAGSDWLDHLWLGPGDKRVPYRTSVMETVRRHGPGWKAFFNAADAWLRVHDRLRHLPPLGRLRTEALRACEEWILARQEASGDWAGIFPPMLNGVLALHVAGHGLDAAPVRRGLEAIERFAVSDREGFRIEACQSPVWDTILALIGLLDSGESPTDPRLVAARRWIEGMQLTNDWGDWKVYDPRGEPGGWAFEYANSWYPDVDDTAAVIVGLLKHDPASRAGETVRRAAAWVASMQNRDGGWAAFDVNNDRLFLNEIPFSDMDSLCDPSSPDVTGRVLEAFGMLDAPHLRAACRRGVAYLRRAQEPEGSWYGRWGVNYVYGTSNVLNGLARQRVPASDPMVARALGWLDSVQNADGGFGEGLESYADRAAMGRGPSTASQTAWGVMGLLAYRAADDAAVRRGIAWLVERQLADGEAQGSWEEEAFTGTGFPRHFYLRYHLYRHYFPLMALGRFCAQGRG

SEQ ID NO: 67 - protein, 710 aa, Aspergillus clavatus
  source 1..710: note = strain: nrrl 1; note = EAW07713.1 prenyltransferase and squalene oxidase repeat protein [Aspergillus clavatus NRRL 1]
MATLTTMATTATMATTEASQPLEAQARTALTKATSYAWEIISNRHWCGELESNVTVTCEHIFFLYVLYQHIDPDEGSQYRQWLLSQQNADGSWGIAPNYPGDVSTSAEAYLALRIIGMSPDSPELFQARTFIRAAGGLSKMRMFTRIFFAEFGLVPWTAIPQLPAEFILVPAHFPISIYRLASWARSNVVPLLIIAHHRPLYPLPNGLHKQNPFLDELWLDPATKPLPYGSLDPTDPLSFVFTILDKALSYLGGLRRCPTRGYARRRCIQWILQHQEKAGDWAGIIPPMHAGIKALWLEGYKLHDEPIQLGLAAIERFTWTDNRGKRLQCCISPVWDTVLMIRALQDTPASLGIKSDPRIADALAWTAENQHRGPEGDWRVYQPNIPVGGWAFEYSNTWYPDIDDTAAAVLAFLTHDPATARSRLVRDAVLWIVGMQNADGGWAAFDHENNRLFLNKIPFSDMESLCDPSTPDVTGRTIECLGMLRDLLMLPAEKAGKKGEKYGYPDGERDAAADSHLLKIINTACARAIPYLIRTQEATGAWYGRWAVNYVYGTCLVLCGLQYFKHDPTFAPEIDTMATRAVKWLRQIQNSDGGWGESVLSYREPWRAGCGPSTPSQTAWALMGLLTVCGGEDRSVQRGVRHLVDTQDDILSKGEGGAAAWTEREFTSTGFPNHFYISYTLYRVYFPITALGRYLSLVEGGKKENGGGA

SEQ ID NO: 68 - protein, 673 aa, Aspergillus flavus
  source 1..673: note = strain: nrrl3357; note = EED48353.1 squalene-hopene-cyclase - putative [Aspergillus flavus NRRL3357]
MTTGHRQFDDGLSERERLIHEAGLTLQRSMDYAYNVVRSDGHWCGEMSSNVTITAEYIFLRQALGLDLKTDGAAYCRHILSQQNSDGSWGLAPEYPGDVSTTTEAYLALKMLGLSTDAPAMQQAKAFVLNAGGVAKVRVFTRIFLATFGLFPWKAVPQLPVELILLPSACPINIYKFASWARGTIAPLLIICHHQPVYALPNGVFAENEYLDELWQDPTNKSEPYSPSIWELLSQGDITGLTFSLLDKLLYQLNGLRSIPLLRSYALKQCMKWILERQEPTGDWAGIFPPMHASVYAFVLEGYKLEDPPVRLGIEALENFAWEDAKGKRVQPCVSPVWDTTLMSIALSDAATPNHQIVDRAIQWIRDRQLLEPRGDWRVYRPRLAPGGFSFEYTNSHYPDIDDSAAIILAQVKHDPISANSSSVIAAATWILGMQNPDGGWAAFDVENDKLFLNKIPFSDMDSLCDTSCADITGRILEAFGLLIRRVPDKDSSQLFQLLPAIRAACRRGIRYLASTQEANGAWFGRWGCNYIYGTSHALCGLAYFLQEDQQVPAMVQPALQWLKSQQNDDGGWGESLLSYQSPERKEQRSTASQTAWALMGLLAHLPHTDIVIERGIRWLVSSQRPVETLGSTWPEPVYTGTGFPNHFYLGYDYYRHYFPMMALGRYLRGVQG

SEQ ID NO: 69 - protein, 738 aa, Aspergillus fumigatus
  source 1..738: note = strain: a1163; note = EDP50814.1 squalene-hopene-cyclase - putative [Aspergillus fumigatus A1163]
MLGAIREPPIDVQIALHSRDDNQTGLVLRGTRRTVDRVLKGLCSSPCFFCSVSLTMATLTTTMATTATMATTEASKPLEAQARTALTKATNYAWEIFSNRHWCGELESNVTVTCEHIFFLYVLYQHIDPGEGSQYRQWLLSQQNSDGSWGIAPNYPGDISTSAEAYLALRIIGMSTDSPELYRARTFIRAAGGLSKMRMFTRIFFAEFGLVPWTAIPQLPAEFILVPAHFPISIYRLASWARSNVVPLLIIAHHRPLYPLPNGLHKQNPFLDELWLDPATKPLPYGSSDPTDPVAFVFTILDKALSYLGGLRRSPTRGYARRRCVQWILQHQEKAGDWAGIIPPMHAGIKALLLEGYKLHDEPIQLGLAAIERFTWADNRGKRLQCCISPVWDTVLMIRALQDTPASLGIKLDPRIADALAWTAENQHRGPEGDWRVYKPNIPVGGWAFEYHNTWYPDIDDTAAAVLAFLTHDPATARSRLVRDAVLWIVGMQNADGGWAAFDHENNQLFLNKIPFSDMESLCDPSTPDVTGRTIECLGMLRDLLMRPAENAENGEKYGYPDGEGDAAADAHLLQIINTACARAIPYLIRSQEATGTWYGRWAVNYVYGTCLVLCGLQYFKHDPKFAPEIQAMAARAVKWLKQVQNSDGGWGESLLSYREPWRAGCGPSTPSQTAWALMGILTVCGGEDRSVQRGVRHLVDTQDDTLSQGDGGAAAWTEREFTIREPLHEASQRIGSD

SEQ ID NO: 70 - protein, 689 aa, Aspergillus fumigatus
  source 1..689: note = strain: af293; note = EAL84865.1 squalene-hopene-cyclase - putative [Aspergillus fumigatus Af293]
MLQTEAITTEGLRVRSLSPDDPLLPRIKQAIKLSGQHSRGEMHSDGHWCGEVKTNATTSAEHVLLCQALGINLDADREAFISWFRCTQGADGGWSTAPDQAGDISVTVEAYLALKILGLSEDDAAMRRARDFAIAAGGVAKVRIFTRIYLALFGLFPWAAVPELPPELILLPSRVPVSIYHWSAWARATVVPLLIISHHRPIYALPGGGKGTSSDYLDELWCDPQNKMIPYNHDEPTAWRSDPFASIFTLADSILHRLDGLRSFNPFRRFALQKCVDWILEHQEDMGDIGDIMPPLHGAMLALRLEGYPLHSGPIHRGLEAIERFAYRDKQGKRIQTTVSAFWDTSLMLIALGDAGMASKPWLTRSLGWLQQHQRLGNYGDWKVNNHGLKAGGFSFGYFNTWYPDVDDTASAVLAMIRQDERLVHSASVLDALNWLLGMQNTDGGWGAFDRDNDKHFLNKIPFSDMDALCDPSTPDVTGHVLEAFGLFLALSKADALADRVVAASRRAIRYLSDTHVLSRGWYGRWGCNYIYGTSAVLCALAYFGSENDALSGVRVMKDAINQAIRWLETVQNPDGGWGETVDSYKDPSRAGSGPSTASQTAWAIMALLPYLPPSTEVIQRGMEYLLRTQTKTASQGATWHEKAYTATGFPKYFYMGYSLYAHYFPMMALGRYAYPCPAWHENWRLKRD

SEQ ID NO: 71 - protein, 671 aa, Aspergillus fumigatus
  source 1..671: note = strain: af293; note = EAL86291.2 squalene-hopene-cyclase - putative [Aspergillus fumigatus Af293]
MATLTTTMATTATMATTEASKPLEAQARTALTKATNYAWEIFSNRHWCGELESNVTVTCEHIFFLYVLYQHIDPGEGSQYRQWLLLQQNSDGSWGIAPNYPGDISTSAEAYLALRIIGMSTDSPELYRARTFIRAAGGLSKMRMFTRIFFAEFGLVPWTAIPQLPAEFILVPAHFPISIYRLASWARSNVVPLLIIAHHRPLYPLPNGLHKQNPFLDELWLDPATKPLPYGSSDPTDPVAFVFTILDKALSYLGGLRRSPTRGYARRRCVQWILQHQEKAGDWAGIIPPMHAGIKALLLEGYKLHDEPIQLGLAAIERFTWADNRGKRLQCCISPVWDTRVYKPNIPVGGWAFEYHNTWYPDIDDTAAAVLAFLTHDPATARSRLVRDAVLWIVGMQNADGGWAAFDHENNQLFLNKIPFSDMESLCDPSTPDVTGRTIECLGMLRDLLMRPAENAENGEKYGYPDGEGDAAADAHLLQIINTACARAIPYLIRSQEATGTWYGRWAVNYVYGTCLVLCGLQYFKHDPKFAPEIQAMAARAVKWLKQVQNSDGGWGESLLSYREPWRAGCGPSTPSQTAWALMGILTVCGGEDRSVQRGVRHLVDTQDDTLSQGDGGAAAWTEREFTSTGFPNHFYISYTLYRVYFPITALGRYLSLIEGGQEKKKKGGGT

SEQ ID NO: 72 - protein, 693 aa, Aspergillus niger
  source 1..693: note = CAK43501.1 unnamed protein product [Aspergillus niger]
MAHQETMASETSISLHTLACDATKLAGTYALRQVREDGHWYGEMKSNATITAEYVFLAQALGFSIEEDRDDLIKYFLSEQNTDGSWSLAYDFPGDVSVTAEAYFALCLLGLDRSHPAMASAREFTLSKGGIAKVRVFTRMFFACFGLFPWSAVPELPAELILLPAAAPMSIYQLASWARATVVPMLVIRHHRPIYALPNGRSSSNEYLDELWVDPTDKMVPYSPSLWSLWNDDLTAFGFTLADNILKALGGLRWFPSRKIALRHCVAWILERQEPEGDIGGIFPPLHAALFALALEGYGLESSPVRRGIDALQNTYAWRDSTGLRIQGCISPILDTILMTIGLIDSSLPAESPLVARSSRYLKAHQQLGNEGDWRVYNGNVPSGGFNFEYFNSWYPDIDDTAAAILAMVKQDPNLLDLGPILSAVQWILGLQNDDGGWAAFDRENNYLFLNKIPFSDMDSFCDPSTADVTGRVIECFGLNGKNPIPRFFIDDMSSATERAIDFLSTEQEADGSWYGRWGSNYIYGTSAVLCGLVYHLEGWDDTYPVMEKRHKVDTHAALDWLKRHQNPDGGWGERLESYYEPRLAGNGPSTASQTAWALMGLLAYLAPTDESITRGIQYLSRTQIKEGELAGSWKEDHYTGTGFPNHFYLCYTLYSQYFPMMALGRYTSLSGYRPLENLESTVEDHKGNSSDC

SEQ ID NO: 73 - protein, 653 aa, Aspergillus niger
  source 1..653: note = CAK45506.1 unnamed protein product [Aspergillus niger]
MMTLREEGHKEGITPGKEQLTSDIEHSLKLATEYALSSIRSDGHWCGELRSNVTITAEYIFLRHALGLDLRTDNAAYCRYILSQQNCDGSWGLAPEYPGDVSTTTEAYLALKLLGTSPDMPAMQQARAFVRKAGGAEKVRVFTRIFLATFGLFPWDAVPQLPVELILLPSSCPINMYTLASWARGTIAPLLIICHHQPVYALPEDYLDELWLDPTDKNVPYGSSLRDLLSRGDITGLAFSVVDNLLYYLNGLRSVPLLRSYARRKCIQWILERQEPTGDWAGIFPPMHASIYAFVLEGYELNDPPVRLGIQALENFAWEDEKGKRIQACVSPVWDTALMSIGLCDAMSPDKQILQQAITWIRNRQLLKPCGDWRIYRSKLAPGGFSFEYENSHYPDVDDTAAIILAQLKQDPQSVASDSVIAAATWILGMQNPDGGWAAFDVENDKLFLNKIPFSDMDSLCDTSCADITGRILEAFGLMMKRELKRPVLSPMLRHACIRGITYLASTQESNGAWFGRWGCNYIYGTCHALGLVAPALQWLKSKQNDDGGWGEPLLSYRTPGTQLQQQSTPSQTAWALMGLLAHLPLTDPAIERGIRWLVCSQQPEKGNGASWPEAVYTGTGFPNHFYLGYDYYRHYFPMMALGRYLQASQAQA

SEQ ID NO: 74 - protein, 673 aa, Aspergillus oryzae
  source 1..673: note = strain: rib40; note = BAE63941.1 unnamed protein product [Aspergillus oryzae RIB40]
MTTGHRQFDDGLSERERLIHEAGLTLQRSMDYAYNVVRSDGHWCGEMSSNVTITAEYIFLRQALGLDLKTDGAAYCRHILSQQNSDGSWGLAPEYPGDVSTTTEAYLALKMLGLSTDAPAMQQAKAFVLNAGGVAKVRVFTRIFLATFGLFPWKAVPQLPVELILLPSACPINIYKFASWARGTIAPLLIICHHQPVYALPNGVFAENEYLDELWQDSTNKSEPYSPSIWELLSQGDITGLTFSLLDKLLYQLNGLRSIPLLRSYALKQCMKWILERQEPTGDWAGIFPPMHASVYAFVLEGYKLEDPPVRLGIEALENFAWEDAKGKRVQPCVSPVWDTTLMSIALSDAATPNHQIVDRAIQWIRDRQLLEPRGDWRVYRPRLAPGGFSFEYTNSHYPDIDDSAAIILAQVKHDPISANSSSVIAAATWILGMQNPDGGWAAFDVENDKLFLNKIPFSDMDSLCDTSCADITGRILEAFGLLIRRVPDKDSSQLFQLLPAIRAACRRGIRYLASTQEANGAWFGRWGCNYIYGTSHALCGLAYFLQEDQQVPAMVQPALQWLKSQQNDDGGWGESLLSYQSPERKEQRSTASQTAWALMGLLAHLPHTDIVIERGIRWLVSSQRPVETLGSTWPEPVYTGTGFPNHFYLGYDYYRHYFPMMALGRYLRGVQG

SEQ ID NO: 75 - protein, 627 aa, Bacillus velezensis
  source 1..627: note = strain: fzb42; note = ABS74269.1 squalene--hopene cyclase [Bacillus velezensis FZB42]
MSTIHENVRSRQKKTISLLRETQNADGSWSFCFEGPILTNAFLILLLTSLGDNDKELIAELAEGIRAKQRPDGTFANYPDDRKGNVTATVQGYAGLLASGLYSRSEAHMIQAERFIISNGGLRNVHFMTKWMLAANGLYPWPALHLPLSFLVIPPTFPLHFYQFSTYARIHFVPMAVTLNKRFSLKNPNVSSLAHLDRHMTKNPFTWLRSDQDENRDLSSLFAHWKRLLQIPAAFHQLGLRTAKTYMLDRIEEDGTLYSYASATIFMVYGLLALGVSRHSPVLRKALAGTKALLTSCGNIPYLENSTSTVWDTALLNYALMKSGISDNDQMITSAARFLRERQQKKVADWAVHNPHAEPGGWGFSNINTNNPDCDDTAAVLKAIPRKLYPASWERGLSWLLSMQNSDGGFSAFEKNVNHPLVRLLPLESAEEAAIDPSTSDLTGRVLHCLGEAGLSSDHPQIEKAVQWLIRHQEEDGSWYGRWGVCYIYGTWAALTGMKACGVSQNHPAVKKAIRWLKSIQNEDGSWGESCKSAEEKTYVPLSYGTLVQTAWAAEALLQYEKTHHQAVTKGISFLIENRHYEGAAFSYPTGIGLPKQFYIRYHSYPYVFSLLALSTFMKMSEKEEEK

SEQ ID NO: 76 - protein, 631 aa, Bacillus wiedmannii
  source 1..631: note = EEK66523.1 Squalene-hopene cyclase [Bacillus wiedmannii]
MNIVIRISKGWVSNLLLYEKVHEEIARRTTALQTMQRQDGTWQFCFEGAPLTDCHMIFLLKLLGRDKEIEPFVKRLASLQTNEGTWKLYEDEVGGNLSATIQSYAALLASKKYTKEDANMKRAEMFINERGGVARAHFMTKFLLAIHGEYEYPSLFHLPTPIMFLQNDSPLSIFELSSSARIHLIPMMVCLNKRFQVGKKLLPNLNHIAGGGGEWFREDRSPMFQTLLSDVKQIISYPLSLHHKGYEEVERFMKERIDENGTLYSYATASFYMIYALLALGHSLQSSMIQKAIAGITSYIWKMEKGNHLQNSPSTVWDTALLSYTLQEAHASKDNKMIQHAAAYVLKKQHTKKADWSVHAPGLIPGGWGFSDVNTTIPDVDDTTAVLRALARSRGNENVDNAWKKGVNWVKGLQNNDGGWGAFEKGVTSNLLANLPIENASDMITDPSTPDITGRVLELFGTYAQNELPEKQKQSAINWLMNVQEENGSWYGKWGICYIYGTWAVMTGLRSLGIPSSNPSMKRAALWLEHIQHEDGGWGESCQSSVEKRFITLPFSTPSQTAWALDALISYHDEETPAIRKGISYLLANPYVNEKYPTGTGLPGGFYIHYHSYAYIYPLLTLAHYIKKYRK

SEQ ID NO: 77 - protein, 631 aa, Bacillus cereus
  source 1..631: note = strain: g9241; note = EAL12758.1 squalene--hopene cyclase [Bacillus cereus G9241]
MNIVIRISKGWVSNLLLYEKVHEEIARRTTALQSMQRQDGTWRFCFEGAPLTDCHMIFLLKLLGRDKEIEPFVKRLASLQTNEGTWKLYEDEVGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLVSDVKKIITYPLSLHHKGYEEVERFMKERIDENGTLYSYATASFYMIYALLALGHSLQSSMIQKAIAGITSYMWKMESGNHVQNSPSTVWDTALLSYALQEAHVLKDNKMLQNATAYLLKKQHTKKADWSVHAPALTPGGWGFSDVNTTVPDVDDTTAVLRVLARSRGNEKVDHAWQKGINWVKGLQNNDGGWGAFEKGVTSHILANLPIENASDMITDPSTPDITGRVLEFFGTYAQNELPEKQKQSAINWLMNVQEENGSWYGKWGICYIYGTWAVLTGLRSLGIPSSDPSLKRAALWLEHIQHEDGGWGESCQSSVEKRFVTLPFSTPSQTAWALDALISYYDKETSVIRKGISYLLSNPYINETYPTGTGLPGGFYIRYHSYAHIYPLLTLAHYAKKYRK

SEQ ID NO: 78 - protein, 620 aa, Bacillus cereus
  source 1..620: note = strain: rock3-42; note = EEL44583.1 Squalene-hopene cyclase [Bacillus cereus Rock3-42]
MSNLLLYEKAHEEIVRRATALQTMQWQDGTWRFCFEGAPLTDCHMIFLLKLLGRDKEIEPFVERVASLQTNEGTWKLYEDEVGGNLSATIQSYAALLASKKYTKEDANMKRAENFIQERGGVARAHFMTKFLLAIHGEYEYPSLFHLPTPIMFLQDDAPFSIFELSSSARIHLIPMMLCLNKRFRVGKKLLPNLNHIAGGGGEWFREDRSPVFQTLLSDVKQIISYPLSLHHKGYEEIERFMKERIDENGTLYSYATASFYMIYALLALGHSLQSSMIQKAIAGITSYIWKMERGNHLQNSPSTVWDTALLSYALQEAQVSKDNKMIQNATAYLLKKQHTKKADWSVHAPALTPGGWGFSDVNTTIPDIDDTTAVLRALARSRGNKNIDNAWKKGGNWIKGLQNNDGGWGAFEKGVTSKLLAKLPIENASDMITDPSTPDITGRVLEFFGTYAQNELPEKQIQRAINWLMNVQEENGSWYGKWGICYIYGTWAVMTGLRSLGIPSSNPSLKRAASWLEHIQHEDGGWGESCHSSVEKRFVTLPFSTPSQTAWALDALISYYDTETPAIRKGVSYLLLNPYVNERYPTGTGLPGAFYIRYHSYAHIYPLLTLAHYLKKYRK

SEQ ID NO: 79 - protein, 620 aa, Bacillus cereus
  source 1..620: note = strain: m1293; note = EEK43841.1 Squalene-hopene cyclase [Bacillus cereus m1293]
MSNLLLYEKVHEEIARRATALQTMQRQDGTWRFCFEGAPLTDCHMIFLLKLLGRDKEIEPFVKRLASLQTNEGTWKLYEDEVGGNLSATIQSYAALLASQKYTKEDANMKRAENFIKERGGVARAHFMTKFLLAIHGEYEYPSLFHVPTPIMFLQNDSPLSIFELSSSARIHLIPMMVCLNKRFRVGKKLLPNLNHIAGGGGEWFREDRSPLFQTLLSDVKQIISYPLSLHHKGYEEVERFMKERIDENGTLYSYATASFYMIYALLALGHSLQSSMIQKAIAGITSYIWKMERGSHLQNSPSTVWDTALLSYALQEAQVPKDHKMIQQTITYLLKKQHTKKADWSVHAPALTPGGWGFSDVNTTVPDVDDTTAVLRVLARSRENEKVNNAWQKGIDWVKGLQNNDGGWGAFEKGVTSKLLANLPIENASDMITDPSTPDITGRVLELFGTYTQNELPEKQKQSAINWLMNAQEENGSWYGKWGICYIYGTWAVMTGLRSLGIPSNNPSLKRAALWLEHIQHEDGGWGESCQSSMEKRFITLPFSTPSQTAWALDALISYYDTETPAIRKGISYLLANPYVNEKYPTGTGLPGGFYIRYHSYAQIYPLLTLAHYTKKYRK

SEQ ID NO: 80 - protein, 620 aa, Bacillus cereus
  source 1..620: note = strain: atcc 4342; note = EEK82938.1 Squalene-hopene cyclase [Bacillus cereus ATCC 4342]
MSNLLLYEKAHEEIARRATALQTMQREDGTWRFCFEGAPLTDCHMIFLLKLLGRDKEIEPFVKRLASLQTNEGTWKLYEDEVGGNLSATIQSYAALLASGKYTKEDANMKRAENFIKERGGVARAHFMTKFLLAIHGEYEYPSLFHVPTPIMFLQNDSPLSIFELSSSARIHLIPMMLCLNKRFRVGKKLLPNLNHIAGGGGEWFREERSPLFQTLLSDVKQIISYPLSLHHKGYEEVERFMKERIDENGTLYSYATASFYMIYALLALGHSLQSSMIQKAIAGITSYIWKMESGNHVQNSPSTVWDTALLSYALQEAHVPKDNKMLQNATAYLLKKQHTKKADWSVHAPALTPGGWGFSDVNTTVPDVDDTTAVLRVLARSKGNEKLDHAWQKGINWVKGLQNNDGGWGAFEKGVTSRILANLPIENASDMITDPSTPDITGRVLEFFGTYAQNELPEKQKQSAINWLMNAQEENGSWYGKWGICYIYGTWAVMTGLRSFGIPSSNPSLKRAALWLEHIQHKDGGWGESCHSSVEKRFVTLPFSTPSQTAWALDALISYYDTETPVIRKGISYLLANPYVNEKYPTGTGLPGGFYIRYHSYAHIYPLLTLTHYIKKYRK

SEQ ID NO: 81 - protein, 620 aa, Bacillus cereus
  source 1..620: note = strain: bdrd-st26; note = EEK99528.1 Squalene-hopene cyclase [Bacillus cereus BDRD-ST26]
MSNLLLYEKAHEEIARRATALQSMQWQDGTWRFCFEGAPLTDCHMIFLLKLLGRDKEIEPFVKRLASLQTNEGTWKLYEDEVGGNLSATIQSYAALLASGKYTKEDANMKRAENFIKERGGVARAHFMTKFLLAVHGEYEYPSLFHLPTPIMFLQNDSPLSIFELSSSARIHLIPMMLCLNKRFRVGKKLLPNLNHIAGGGGEWFREERSPLFQTLVSDVKQIISYPLSLHHKGYEEVERFMKERIDENGTLYSYATASFYMIYALLALGHSLQSSIIQNAITGITSYIWKMESGNHLQNSPSTVWDTALLSYALQEAHVPKDNKMLQNATAYLLKKQHTKKADWSVHASALTPGGWGFSDVNTTVPDVDDTTAVLRVLARSRGNEKVDHAWQKGINWVKGLQNNDGGWGAFEKGVTSNILAKLPIENASDMITDPSTPDITGRVLEFFGTYAQNELPEKQKQSAINWLMNVQEENGSWYGKWGICYIYGTWAVMTGLRSFGIPSSNPSLKRAALWLEHIQHKDGGWGESCHSSVEKRFVTLPFSTPSQTAWALDALISYYDTETPIIRKGISYLLANPYVNEKYPTGTGLPGGFYIRYHSYAHIYPLLTLAHYIKKYRK

SEQ ID NO: 82 - protein, 620 aa, Bacillus cereus
  source 1..620: note = strain: r309803; note = EEK77935.1 Squalene-hopene cyclase [Bacillus cereus R309803]
MNNLLLYEKVHEEIARRATALQTMQQQDGTWRFCFEGAPLTDCHMIFLLKLLGRDKEIEPFVKRLASLQTNEGTWKLYEDEVGGNLSATIQSYAALLASKKYTKEDANMKRAENFIKERGGVARAHFMTKFLLAIHGEYEYPSLFHLPTPIMFLQNDSHLSIFELSSSARIHLIPMMLCLNKRFRVGKKLLPSLNHIAGGGGEWFREDRSPLFQTLVSDVKQIISYPLSLHHKGYEEVERFMKERIDENGTLYSYATASFYMIYALLALGHSLQSTMIQKAITGITSYIWKMESGNHLQNSPSTVWDTALLSYALQEAHVPKDNKMIQHAATYLLKKQHTQKADWSVHAPALTPGGWGFSDVNTTIPDVDDTTAVLRALARSRGNEKVDNAWPKGINWVKGLQNNDGGWGAFEKGVTSNILANLPIENASDMITDPSTPDITGRVLEFFGKYAQNELPEKQKQSAINWLMNVQEENGSWYGKWGICYIYGTWAVMTGLRSLGIPSSNPSMKRAALWLEHIQHEDGGWGESCHSSVEKRFVTLPFSTPSQTAWALDALISYYDKETSIIRKGISYLLANPYVNEKYPTGTGLPGGFYIRYHSYAHIYPLLTLAHYIKKYRK

SEQ ID NO: 83 - protein, 620 aa, Bacillus cereus
  source 1..620: note = strain: ah1271; note = EEL81079.1 Squalene-hopene cyclase [Bacillus cereus AH1271]
MSNLLLYEKVHEEIARRTTALQTMQRRDGTWRFCFEGAPLTDCHMIFLLKLLGRDKEIEPFVKRLASLQTNEGTWKLYEDEVGGNLSATIQSYAALLASKKYTKEDANMKRAEMFINERGGVARAHFMTKFLLAVHGEYEYPSLFHLPTPIMFLQSDSPLSIFELSSSARIHLIPMMLCLNKKFRIRKKLLPNLNHISGGGGEWFRGNRSPLFQTLVSDVKQIISYPLSLHHKGNEEVERFMKERIDENGTLYSYATASFYMIYALLALGHSLQSTMIQKAITGITSYIWNMESGNHLQNSPSTVWDTALLSYALQEAHVPKDTNMLQHATAYLLKKQHTKKADWSVHAPALAPGGWGFSDVNTTIPDVDDTTAVLRALARSRGSEKVDYVWEKGINWVKGLQNNDGGWGAFEKGVTSNLLANLPIENASDMITDPSTPDITGRVLELFGTYAQNELPEKQTQSAINWLMNVQEKNGSWYGKWGICYIYGTWAVMTGLRSLGIPSSNPSLKRAALWLEHIQHEDGGWGESCHSSVEKRFVTLPFSTPSQTAWALDALISYYDKETPAIRKGISYLLANRYVNEKYPTGTGLPGGFYICYHSYAHIYPLLTLAHYIKKYRK

SEQ ID NO: 84 - protein, 620 aa, Bacillus cereus
  source 1..620: note = strain: m1550; note = EEK88307.1 Squalene-hopene cyclase [Bacillus cereus m1550]
MSNLLLYEKVHEEIARRTTALQTMQRQDGTWQFCFEGAPLTDCHMIFLLKLLGRDKEIEPFVKRLASLQTNEGTWKLYEDEMGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLVSDVKKIITYPLSLHHKGYEEVERFMKERIDENGTLYSYATASFYMIYALLALGHSIQSPIIQKAITGITSYIWKMERGSHLQNSPSTVWDTALLSYVLQEAQVPKASKVIHNASAYLLRKQQTKKVDWSVHAPDLFPGGWGFSDVNTTIPDIDDTTAALRALARSRGNENVDTAWKRAVNWVKGLQNNDGGWGTFEKGVTSRILANLPIENASDMITDPSTPDITGRVLEFFGTYTQNELPEKQKQSAINWLMNVQEENGSWYGKWGICYIYGTWAVLTGLRSLGIPSSDPSVKRAALWLEHIQHEDGGWGESCQSSVEKRFVTLPFSTPSQTAWALDALISYYDKETPVIRKGISYLLSNSYINEKYPTGTGLPGGFYIRYHSYAHIYPLLTLAHYAKKYRK

SEQ ID NO: 85 - protein, 620 aa, Bacillus cereus
  source 1..620: note = strain: f65185; note = EEL63943.1 Squalene-hopene cyclase [Bacillus cereus F65185]
MSNLLLYEKVHEEIARRTTALQTMQRQDGTWRFCFEGAPLTDCHMIFLLKLLGRDKEIEPFVKRLASLQTNEGTWKLYEDEVGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLVSDVKKIITYPLSLHHKGYEEVERFMKERIDENGTLYSYATASFYMIYALLALGHSIQSPIIEKAIMGITSYIWKVERGSHLQNSPSTIWDTALLSYALQEAQVPKASKVIQNASAYLLRKQQTKKVDWSVHAPDLFPGGWGFSDVNTTIPDIDDTTAVLRALARSRGNEHVDNAWKRAVNWVKGLQNNDGGWGAFEKGVTSRILANLPIENASDMITDPSTPDITGRVLEFFGTYTQNELPEKQKQSAINWLMNVQEENGSWYGKWGICYIYGTWAVLTGLRSLGIPSSDSSLKRAVLWLEHIQHEDGGWGESCQSSVEKRFVTLPFSTPSQTAWALDALISYYDKETPVIRKGISYLLSNPYINEKYPTGTGLPGGFYIRYHSYAHIYPLLTLAHYAKKYRK

SEQ ID NO: 86 - protein, 620 aa, Bacillus cereus
  source 1..620: note = strain: rock4-18; note = EEL59884.1 Squalene-hopene cyclase [Bacillus cereus Rock4-18]
MNALLLYEKVHEEIARRTTALQTMQRQDGTWRFCFEGAPLTDCHMIFLLKLLGRDKEVEPFVKRLASLQTNEGTWKLYDDEMGGNLSATIQSYAALLASKKYTKEDANMKRAEMFITERGGVARAHFMTKFLLAIHGEYEYPSLFHLPTPIMFLQNDSPLSIFELSSSARIHLIPMMLCLNKRFRVGKKLLPNLNHIAGGGGEWFREDQSPMFQTLLGNVKQIISYPLSLHHKGNEEVERFMKERIDENGTLYSYASASFYMIYALLALGHSIQSPMIQKAITGITSYIWKMERGNHLQNSPSTVWDTALLSYALQEARVSKESKMIQNASAYLLKKQHKKKADWSVHAPVLIPGGWGFSDVNTTVPDVDDTTAVLRALAQSRGNGNVDDAWKKGTNWIKGLQNNDGGWGAFEKGVTSKLLANLPIENASDMITDPSTPDITGRVLEFFGTYTQNELPEKQKQSAINWLMNEQEENGSWYGKWGICYIYGTWAVMTGLRALGITSAHPSLKRATLWLEHIQHEDGGWGESCQSSVEKRFATLPFSTPSQTAWALDALISYYDKETPAIRKGISYLLANPYVNEKYPTGTALPGGFYIHYHSYAHIYPLLTLAHYAKKYKK

SEQ ID NO: 87 - protein, 617 aa, Bacillus cereus
  source 1..617: note = strain: rock4-18; note = EEL59884.2 Molybdopterin converting factor - subunit 1 [Bacillus cereus Rock4-18]
MLLYEKVHEEIARRTTALQTMQRQDGTWRFCFEGAPLTDCHMIFLLKLLGRDKEVEPFVKRLASLQTNEGTWKLYDDEMGGNLSATIQSYAALLASKKYTKEDANMKRAEMFITERGGVARAHFMTKFLLAIHGEYEYPSLFHLPTPIMFLQNDSPLSIFELSSSARIHLIPMMLCLNKRFRVGKKLLPNLNHIAGGGGEWFREDQSPMFQTLLGNVKQIISYPLSLHHKGNEEVERFMKERIDENGTLYSYASASFYMIYALLALGHSIQSPMIQKAITGITSYIWKMERGNHLQNSPSTVWDTALLSYALQEARVSKESKMIQNASAYLLKKQHKKKADWSVHAPVLIPGGWGFSDVNTTVPDVDDTTAVLRALAQSRGNGNVDDAWKKGTNWIKGLQNNDGGWGAFEKGVTSKLLANLPIENASDMITDPSTPDITGRVLEFFGTYTQNELPEKQKQSAINWLMNEQEENGSWYGKWGICYIYGTWAVMTGLRALGITSAHPSLKRATLWLEHIQHEDGGWGESCQSSVEKRFATLPFSTPSQTAWALDALISYYDKETPAIRKGISYLLANPYVNEKYPTGTALPGGFYIHYHSYAHIYPLLTLAHYAKKYKK

SEQ ID NO: 88 - protein, 620 aa, Bacillus cereus
  source 1..620: note = strain: eel69857.1; note = Bacillus cereus EEL69857.1
MSGVLLYDKVHEEIERRTTALQTMQRQDGTWQFCFEGALLTDCHMIFLLKLLGRNDEIEPFVKRLASLQTNEGTWKLYEDEKGGNLSATIQAYAALLASEKYSKEDMNMRRAEMFIKEHGGVSRAHFMTKFLLAIHGEYEFPALFHFPTPILFLQDDSPLSIFGLSSSARIHLIPMMICMNKRFRVEKKLLPNLNHIAGGGGQWFREERSPLFQSLLGDVKKVISYPLSLHHKGYEEVERFMKERIDENGTLYSYASATFYMIYALLALGHSIQSPIIEKAVTGLKSYIWKMDRGSHLQNSPSTVWDTALLSYSLQEAKVTNENKMIQRATEYLLQKQQTKKVDWSVHASSLVAGGWGFSDVNTTIPDIDDTTAALRALARSRGNDRVDDAWGRGVEWVKGLQNNDGGWGAFERGVTSKLLSNLPIENASDMITDPSTPDITGRVLELFGTYAPNELLEEQKKKAIKWLMDVQEQNGSWYGKWGICYIYGTWATMTGLRALGVPSTHPSLKKAASWLEHLQHEDGGWGESCQSSVEKKFISLPFSTPSQTAWALDALISYYDQETPIIRKGITYLLAQSTMNEKYPTGTGLPGGFYIRYHSYGHIYPLLALAHYVKKYRK

SEQ ID NO: 89 - protein, 620 aa, Bacillus cereus
  source 1..620: note = strain: ah1273; note = EEL92663.1 Squalene-hopene cyclase [Bacillus cereus AH1273]
MSGVLLYDKVREEIERRTTALQTMQRQDGTWSFCFEGALLTDCHMIFLLKLLGRNDEIEPFVKRLASLQTNEGTWKLYEDENGGNLSATIQAYAALLASEKYSKEDINMRRAEMFIKEHGGVSRAHFMTKFLLAIHGEYEFPTLFHFPTPILFLQDDSPLSIFELSSSARIHLIPMMICMNKRFRVEKKLLPNLNHIAGEGGQWFREERSPLFQSFVGDVKKVIAYPLSLHHKGYEEVERFIGERIDENGTLYSYASATFYMIYALLALGHSIQSPIIEKAVIGLKSYIWKMDRGSHLQNSPSTVWDTALLSYSLQEANVMKENKMIQKATEYLLQRQQTKRMDWSVHAPSIMAGGWGFSDVNTTIPDVDDTTAALRALARSRGSSRVDSAWERGVEWLKGLQNNDGGWGAFERGVTSRILANLPIENASDMITDPSTPDITGRVLEFFGTYAPNELPEEQKKKAVKWLMDVQELNGSWYGKWGICYIYGTWAAMTGLRALGVPSSHPSLKKAASWLEHLQYEDGGWGESCQSSVEKKFISLPFSTPSQTAWALDALISYYDQETPIIRKGISYLLAQPTMNEKYPTGTGLPGGFYIRYHSYGHIYPLLALAHYIKKYKK

SEQ ID NO: 90 - protein, 619 aa, Bacillus cereus
  source 1..619: note = strain: rock3-44; note = EEL49968.1 Squalene-hopene cyclase [Bacillus cereus Rock3-44]
MLLYEKVHEEVKEKMAALQAMQQQDGTWRFCFEGSPLTDCYMIFLLTLLGQDQEIEPFVARLAALQTNEGTWKLYEDEPDGNLSATIQAYAALLVSKMYKKEDINMKRAEVFIRKQGGITKAHFMTKFLLALHGGYEYPPLFHFPTPILFLSEDSPLSIFELSSSARIHLIPMMLCMNKRFTVSKKMLPNLDYISGGSKEQWFREERSPLFQTLLRDVTKFLSYPLSLHYKGDKAAERFMIERIDTNGTLYSYASATFYMIYALLALGHSIQSPLISNAVLGLKTYVWNMDRWAHLQNSPSTVWDTALLSYSLQEARVPHDNEMIQKAINYLLQKQHKEKKDWSVHAPTLDAGGWGFSDVNTTIPDVDDTTAVLRALAGSRQGNPKVESAWRKGIEWVKGLQNSDGGWAAFEKGVTSKVLTHLPLDNSGDMITDPSTVDITGRVLEFFGTYAPNELQGDQKDRAIRWLIYTQEKNGSWHGKWGVCYIYGTWAALTGLRAVGVPSNHIALQKAATWLESIQHSDGGWGESCRSSVEKKFISLPFSTPSQTAWALDALIACYDSETPTIRKGISYLLKHSTKHQEYPTGTALANGFYIRYHSYHHIFPLLTFAHYIKKYRK

SEQ ID NO: 91 - protein, 617 aa, Bacillus cereus
  source 1..617: note = strain: e33l; note = AAU16998.1 squalene-hopene cyclase [Bacillus cereus E33L]
MLLYEKVHEEIVRRATALQTMQWQDGTWRFCFEGAPLTDCHMIFLLKLLGREKEIEPFVERIASLQTNEGTWKLYEDEVGGNLSATIQSYAALLASKKYTKEDANMKRAENFIKERGGVARAHFMTKFLLAIHGGYEYPSLFHLPTPIMFLQNDSPLSIFELSSSARIHLIPMMLCLNKRFRVGKKLLPNLNHIAGGGGEWFREDRSPVFQTLISDVKQIISYPLSLHHKGYEEIERFMKERIDENGTLYSYATASFYMIYALLALGHSPQSSMIQKAIAGLTSYIWKMGRGSHLQNSPSTVWDTALLSYALQEARVSKDNKMIQNATAYLLKKQHTKKADWSVHAPALIPGGWGFSDVNTTIPDIDDTTAVLRALARSRGNKNIDNAWQKGVNWIKGLQNNDGGWGAFEKGVTSKLLANLPIENASDMITDPSTPDITGRVLEFFGTYAQNGLPEKQKQSAINWLMNAQEENGSWYGKWGICYIYGTWAVMTGLRSLGIPSSNPSLKRAASWLEYIQHEDGGWGESCHSSVEKRFVTLPFSTPSQTAWALDALISYYDTETPAIRKGVSYLLSNPYVNERYPTGTGLPGAFYIRYHSYAHIYPLLTLAHYLKKYRK

SEQ ID NO: 92 - protein, 617 aa, Bacillus cereus
  source 1..617: note = strain: atcc 10987; note = AAS42477.1 squalene-hopene cyclase [Bacillus cereus ATCC 10987]
MLLYEKAHEEIARRATALQTMQRQDGTWRFCFEGAPLTDCHMIFLLKLLGRDKEIEPFVKRLASLQTNEGTWKLYEDEVGGNLSATIQSYAALLASKKYTKEDANMKRAENFIKERGGVARAHFMTKFLLAIHGEYEYPSLFHLPTPIMFLQNDSPLSIFELSSSARIHLIPMMVCLNKRFRVGKKLLPNLNHIAGGGGEWFREDRSPLFQTLLSDVKQIISYPLSLHHKGYEEVERFMKERIDENGTLYSYATASFYMIYALLALGHSLQSSLIQKAIAGITSYIWKMERGSHLQNSPSTVWDTALLSYALQEAHVPKDHKMIQQTITYLLKKQHTKKADWSVHALALTPGGWGFSDVNTTIPDVDDTTAVLRALARSRGNENIDNAWKKGVNWIKGLQNNDGGWGAFEKGVTSKLLANLPIENASDMITDPSTPDITGRVLELFGTYTQNELPKKQKQSAINWLMNVQERNGSWYGKWGICYIYGTWAVMTGLRSLGIPSNNPSLKRAALWLEHIQHEDGGWGESCQSSVEKRFVTLPFSTPSQTAWALDALISYYDKETPTIRKGVSYLLANPYVNEKYPTGTGLPGGFYIRYHSYAQIYPLLTLAHYTKKYQK

SEQ ID NO: 93 - protein, 617 aa, Bacillus cereus
  source 1..617: note = strain: g9842; note = ACK95843.1 putative squalene-hopene cyclase [Bacillus cereus G9842]
MLLYEKVYEEIARRTTALQTMQRQDGTWRFCFEGAPLTDCHMIFLLKLLGRDKEIEPFVKRLASLQTNEGTWKLYEDEVGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLLSEVKKIITYPLSLHHKGYEEVERFMKERIDENGTLYSYATASFYMIYALLALGHSIQSPIIQKAITGIASYIWKMERGSHLQNSPSTVWDTALLSYALQEAQVPKASKVIQNASAYLLRKQQTKKVDWSVHAPNLFPGGWGFSDVNTMIPDIDDTTAVLRALARSRGDENVDNAWKRAVNWVKGLQNNDGGWGAFEKGVTSRILANLPIENASDMITDPSTPDITGRVLEFFGTYAQNELPEKQKQSAINWLMNVQEENGSWYGKWGICYIYGTWAVLTGLRSLGIPSSDPSLKRAALWLEHIQHEDGGWGESCQSSVEKRFVTLPFSTPSQTAWALDALISYYEKETPIIRKGISYLLSNPYVNEKYPTGTGLPGGFYIRYHSYAHIYPLLTLAHYTKKYRK

SEQ ID NO: 94 - protein, 486 aa, Bacillus coahuilensis
  source 1..486: note = strain: m4-4; note = ZP_03226483.1 squalene-hopene cyclase [Bacillus coahuilensis m4-4]
MAEAISYPRRVHIITTKFPVNFYDFSVFGRSNIAPILLLADSKFQIPKTTETPDISHLYVRELYWWSEDRGWNGFTKAINKGVNNLIGLPNELHTLGRKQAENYMLDRLEDDGTLLSYYSSTFFMIYALLSVGYTKDHKVIKKAARGLLSMNTTVKDTIHIQYTTAHIWNTSLISHALQTAGASPDDTMVMRANHYLLQRQHTKFGDWAIYQPNLGPGGWGFSHSNTFNPDVDDTTASLRSIQNSLHSHPNYQSSWYRGLSFTLGMQNQDGGFPAFEKGVDKTFLHLLPVQGAEFLLTDPSTPDLTGRTLEFLGESAHLYKDSGAIKRGVNWLIENQRRDGSWYGRWGICYIYGTWAALTGLQAVGVSKEHPSVQEGIDWLKSIQQDDGGWGESCESDSQKTYIPLSKSTVTQTAWAVDALIAYEKEETVEIKKGMEYLLENWNHEDWTMDYPMGQGMAKAFYIHYHSYRYVFPLLTMGHYMRKFM

SEQ ID NO: 95 - protein, 619 aa, Bacillus cytotoxicus
  source 1..619: note = strain: nvh 391-98; note = ABS22481.1 squalene/oxidosqualene cyclase [Bacillus cytotoxicus NVH 391-98]
MVLYGRVCAEIERTITALHTMQQQDGAWRFCFEGSPLTDCHMIFLLRLLEKEEEIEPFVARLTSIQTNEGTWKLYEDERAGNVSTTIQAYAALLASGMYTKEDVNMKRAEAFIQERGGIARSHFMTKFLLALHGGYEYPRMFYFPTPILFLPEDSPLSIFELSSSARIHLIPMMICMNKRFTVSKTILPNLDHISGSSKSEWFREDRSSLFETILGEVKKFVTYPLSLHHKGDKEAERFMIERIDRNGTLYSYASATFYMIYALLALGHHIQSPLIQQAVAGLRTYKWHMEAGIHLQNSPSTVWDTALLSYALQEANVNESTPMIQTATEYIWQRQHHEKKDWSLHAPTLSPGGWGFSDVNTTIPDVDDTTAALRALARSRKRNRRIEEAWKKGVNWVKGLQNKDGGWAAFEKGVTNRFLTHLPLENSGDMMTDPSTADITGRVLEFFGTYAPNELQDHQKNRAITWLMDVQENNGSWYGKWGVSYIYGTWAALTGLRAVGVANTHPALKKAVMWLERIQHRDGGWGESCRSSIEKRFVPLSFSTPSQTAWAIDALISYYDEETPVIRKGISYLLEHAASHQEYPTGTGLPNGFYIRYHSYSYMYPLLTFAHYINKYRK

SEQ ID NO: 96 - protein, 629 aa, Bacillus licheniformis
  source 1..629: note = strain: dsm 13 = atcc 14580; note = AAU23777.1 SqhC [Bacillus licheniformis DSM 13 = ATCC 14580]
MRSILEDVKAFRQKTLAELQNRQRSDGSWRFCFEGPVMTDSFFILMLTSLGDQDSSLIASLAERIRSRQSEDGAFRNHPDERAGNLTATVQGYTGMLASGLYDRKAPHMQKAEAFIKDAGGLKGVHFMTKWMLAANGLYPWPRAYIPLSFLLIPSYFPLHFYHFSTYARIHFVPMAITFNRRFSLKNNQIGSLRHLDEAMSKNPLEWLNIRAFDERTFYSFNLQWKQLFQWPAYVHQLGFEAGKKYMLDRIEEDGTLYSYASATMFMIYSLLAMGISKNAPVVKKAVSGIKSLISSCGKEGAHLENSTSTVWDTALISYAMQESGVPEQHSSTSSAADYLLKRQHVKKADWAVSNPQAVPGGWGFSHINTNNPDLDDTAAALKAIPFQRRPDAWNRGLAWLLSMQNKDGGFAAFEKDVDHPLIRNLPLESAAEAAVDPSTADLTGRVLHLLGLKGRFTDNHPAVRRALRWLDHHQKADGSWYGRWGVCFIYGTWAALTGMKAVGVSANQTSVKKAISWLKSIQREDGSWGESCKSCEAKRFVPLHFGTVVQSSWALEALLQYERPDDPQIIKGIRFLIDEHESSRERLEYPTGIGLPNQFYIRYHSYPFVFSLLASSAFIKKAEMRETY

SEQ ID NO: 97 - protein, 620 aa, Bacillus mycoides
  source 1..620: note = strain: dsm 2048; note = EEL98438.1 Squalene-hopene cyclase [Bacillus mycoides DSM 2048]
MSGVLLYDKVHEEIERRTTALQTMQRQDGTWQFCFEGALLTDCHMIFLLKLLGRNDEIEPFVKRLVSLQTNEGTWKLYEDEKGGNLSATIQAYAALLASERYSKEAMNMRRAEMFIKEHGGVSRAHFMTKFLLAIHGEYEFPALFHFPTPILFLQDDSPLSIFGLSSSARIHLIPMMICMNKRFRVEKKLLPNLNHIAGGGGQWFREERSPLFQSFLGDVKKVISYPLSLHHKGYEEVERFMKERIDENGTLYSYASATFYMIYALLALGHSIQSPIIEKAVTGLKSYIWKMDRGSHLQNSPSTVWDTALLSYSLQEAKVTNENKMIQRATEYLLQKQQTKKVDWSVHASSLVAGGWGFSDVNTTIPDIDDTTAALRALARSRGNDRVDDAWGRGVEWVKGLQNNDGGWGAFERGVTSKLLSNLPIENASDMITDPSTPDITGRVLELFGTYAPNELLEEQKKKAIKWLMDVQEQNGSWYGKWGICYIYGTWATMTGLRALGVPSTHPALKKAASWLEHLQHEDGGWGESCQSSVEKKFISLPFSTPSQTAWALDALISYYDQETPIIRKGISYLLAQSTMNEKYPTGTGLPGGFYIRYHSYGHIYPLLALAHYVKKYRK

SEQ ID NO: 98 - protein, 619 aa, Bacillus pseudomycoides
  source 1..619: note = EEM04821.1 Squalene-hopene cyclase [Bacillus pseudomycoides]
MLLYEKVRQEVERKVTALRTTQYQDGAWRFCFEGSPLTDCHMIFLLRLLGQNGEMEPFVTRVASLQTNEGTWKLYEDESVGNLSTTINAYVALLASGRYTKEDINMKRAEAFIRRQGGITKAHFMTKFLLALHGGYEYPSLFHFPTPMLFLPEDSPLSIFELSSSARIHLIPMMICMNKRFTVSKTIFPNLDYISGGSKKQWFREERSPLFQTLLGDVKKFLSYPLSLQHKGYKEAERFMIERIETNGTLYSYASATFYMIYALLALGHSIQSPLISNAVLGLKSYIWNMNKGTHLQNSPSTVWDTALLSYSLQEAGVPNDNQMIQKATDYLLQKQHKEKKDWSVHAPSLDAGGWGFSDVNTTIPDIDDTTAALRAIARSREGNQRIEEDWRKGIEWVKGLQNIDGGWAAFERGVTSHFLTHLPLDNAGDMTTDPSTSDITGRVLEFFGTYAPHQLKDDQKDRAIKWLMQAQEKNGSWYGKWGVCYIYGTWAVLTGLRAVGVPSNHTALQKAATWLERIQHNDGGWGESCRSSIEKHFISLPFSTPSQTAWALDALITFYDTETPVIRKGISYLLAHLNQNQDYPTGIGLPDGFYIRYHSYHHIFPILTFAHYIKKYMK

SEQ ID NO: 99 - protein, 619 aa, Bacillus pseudomycoides
  source 1..619: note = strain: dsm 12442; note = EEM16144.1 Squalene-hopene cyclase [Bacillus pseudomycoides DSM 12442]
MLLYEKVRQEVERKVTALRTMQYQDGAWRFCFEGSPLTDCHMIFLLRLLGQNGEMEPFVTRVASLQTNEGTWKLYEDESVGNLSTTINAYVALLASGRYTKEDINMKRAEAFIRRQGGITKAHFMTKFLLALHGGYEYPSLFHFPTPMLFLPEDSPLSIFELSSSARIHLIPMMICMNKRFTVSKTILPNLDYISGGSKKQWFREERSSLFQRLLGDVKKFLSYPLSLQHKGYKEAERFMIERIETNGTLYSYASATFYMIYALLALGHSIQSPLISNAVLGLKSYIWNMNKGTHLQNSPSTVWDTALLSYSLQEAGVPNDNQMIQKATDYLLQKQHKEKKDWSVHAPSLDAGGWGFSDVNTTIPDIDDTTAALRAIARSREGNQRIEEAWRKGIEWVKGLQNIDGGWAAFERGVTSHFLTHLPLDNAGDMTTDPSTSDITGRVLEFFGTYAPHQLKDDQKDRAIKWLMQAQEKNGSWYGKWGVCYIYGTWAALTGLRAVGVPSNHTALQKAATWLERIQHNDGGWGESCRSSIEKHFISLPFSTPSQTAWALDALITFYDTETPVIRKGISYLLAHLNQNQDYPTGIGLPDGFYIRYHSYHHIFPILTFAHYIKKYMK

SEQ ID NO: 100 - protein, 624 aa, Bacillus pumilus
  source 1..624: note = strain: safr-032; note = ABV62529.1 squalene-hopene cyclase [Bacillus pumilus SAFR-032]
MQDFKTKVNVYMDELHMQMQHRQREDGAFVFCFEGSMMTNAFLIMLLKAVGDTDQALVHQLAEAIREKQNEDGSFSLYHDQAGHVTATVQGYCGMLVSGRYQQDEPHMEKAARYIRSKGGLKDVHFMTKWMLAVNGMHPWPYFYAPLSFLLIPTYFPLHFYHLSAYARIHFVPMMIALNKRYTSHEQFPSLSHLDANMSKNPFDWFMAREERSTHHFLAYMRSYTALDSRFDFFGYEAAKRYMFDRLEKDGTLYSYLSASIFMVYALMSLGYSPGHHLILKAVKGMKQLVTDCGGKKYAENSTSTVWDTALVSYASQRAGRTQDDPVIKKSFTYLLNRQQMKKADWAIHNRHAAPGGFGFSDLNTNNPDCDDTQIVLKAIPQTYAPVQWKRGFDWLLSMQNRDGGFSAFEKNQDHFLLRHLPLESAEDAAIDPSTPDITGRVLHLIASEENDKSPLMQRQKDHCVKWLLDHQEKDGSWYGRWGVCYIYGTWAALTGLKASGIPSSHPAVQKACRFLKTIQLEDGSFGESCKSSEVKRYVPLPFGTVVQTAWAAEALLQYVQPDDKSILKAISFLIQHQHSSKALHYPVGIGLPKQFYITYHSYPFVFPMMACSTFLEEMRRKNE

SEQ ID NO: 101 - protein, 624 aa, Bacillus pumilus
  source 1..624: note = strain: atcc 7061; note = EDW21137.1 squalene-hopene cyclase [Bacillus pumilus ATCC 7061]
MQDFKTKVNEYIDELHMQLQRRQREDGAFVFCFEGPMMTNAFLIMLLKAVGDSDQALVHQLAEAIREKQNEDGSFSLYHDQAGHVTATVQGYCGMLVSGRYQQDEPHMEKAAHFIRSNGGLKDVHFMTKWMLAVNGMHPWPYFYAPLSFLLIPTYFPLHFYHLSAYARIHFVPMMIALNKRYTSHEQFPSLAHLDANMSKNPFDWFMAREERSTHHFLAYMRSYTALDSRLDFFGYEAAKRYMFDRLEKDGTLYSYLSASIFMVYALMSLGYSPGHHLILKAVKGMKQLVTDCGGRKYAENSTSNVWDTALVSYASQQAGRTQDDPVIKKSFTYLLNRQQMKKADWAIHNRHAAPGGFGFSDLNTNNPDCDDTQIVLKAVPQTYAPVQWKRGFDWLLSMQNQDGGFSAFEKNQNHFLLRHLPLESAEDAAIDPSTPDIAGRVLHLIALEENSMSPLMQRQKDHCVKWLLDHQEKNGSWFGRWGVCYIYGTWAALTGLKTAGISSSHSAVQKACRFLKTIQLEDGSFGESCKSAEVKRYVPLPFGTVVQTAWAAEALLQYVQPDDKVILKAISFLIQHQHSSEALHYPVGIGLPKQFYITYHSYPFVFPMMACSTFLEEMRRKNE

SEQ ID NO: 102 - protein, 638 aa, Bacillus sp.
  source 1..638: note = strain: nrrl b-14911; note = EAR64404.1 SqhC [Bacillus sp. NRRL B-14911]
MKNRNKGAGCMQLVKSEIERLKQQLLSEQTPDGSWNHPFDTGCMTDIYMIVLLRTLEEEDEEELIKELAKGILSRQGKDGAWRLFHDHHEGSLSLTIEAYYALLYSGYYEKNHPALVKARRVITKGGGLKKAGMYTKIMLALTGQYPWPLLFPVPMEVILLPRSFPLNMYDISVFGRSNLIPVILLGNKKFSRKTALSPDLGDLSVRDDDDPWPELRSAEWRSLTSFLAAGVKALVGIPRQIRAWSIEKAREYMQSHTEPDGTLYNYFSSTFYMIFALLALGGGPEEPAIRNAVAGLKRMTVKADGRTHIQYTTAAVWNTALISHALQEAGVPPKENAIQKANQYLAGQQHRRFGDWIVHNTKAEPGGWGFSRFNTINPDVDDTTAALRSLYQPAREKPHYDDIWKKGLLWTLSMQNRDGGWPAFERNVDKKLLHLLPIQGAEFILTDPSTADLTGRTLEFLGKAGYADASLPPIKKAVKWLKKHQEPNGSWYGRWGICYIYGTWAAVTGMAAVGVTLEDKSMKKGIDWLLSIQNEDGGWGESCRSDMEKKYIPLKESTLTQTAWAVDALAAAGMADSTPSRKGAAFLVREGKRKDWTADYPMGQGMANFFYIHYHSYRCIWPLLALSHYIEKSEAPD

SEQ ID NO: 103 - protein, 625 aa, Bacillus sp.
  source 1..625: note = strain: sg-1; note = EDL66148.1 squalene-hopene cyclase [Bacillus sp. SG-1]
METLIDPEISRLTQRLLEDQEEDGAWRYCFENSLMTDAYMIVLIRSLGIKKERLVQELADRLLSQQEEKGFWKIYRDEVEGNLSATVEAYFALLWSGAVKEKDENMVKARDCILSGGGLDKVHSMTKFMLAAHGQYPWDRFFPVPVEVILLPTYFPVSFTDFSAYARVHLAPLLLLKSERYIRKTSTTPDLSYLLKDQEDFSFFREEERSFIEYVTSGVEAIAAFPANLNDLAKKTALNYMLARLEPDGSLYSYFSSSFYMIIALLSQGYSRKDPLVVNAIKALISYQCKGDGYPHIQNSPSTIWDTALISHALQSSGVDSRNAQILKASHYLYRHQHTQKGDWASEAPQTAPGGWGFSESNTINPDVDDTTAALRALKLDAYTDPVKRMAWNRGVKWALSMQNKDGGWPAFEKNKNKDILSWVPMDGAEDAALDRSCADLTGRTLEFLGNDAGMGRENSQVLKGIEWLMNNQENDGSWYGKWGICYIYGTWAALTGMMAAGMSADHQSIIKAIKWLYQIQNSDGGWGESCRSDKERKYISLGASTPSQTAWALDALISINDHPTKEIDRGIESLVRLLNTDDWRKEYPTGAGLPGRFYIHYHSYPYIWPLLALSNYKTKFLEVR

SEQ ID NO: 104 - protein, 632 aa, Bacillus subtilis
  source 1..632: note = sp|Q796C3.1|SQHC_BACSU RecName: Full=Sporulenol synthase - AltName: Full=Tetraprenyl-beta-curcumene cyclase
MGTLQEKVRRFQKKTITELRDRQNADGSWTFCFEGPIMTNSFFILLLTSLDEGENEKELISSLAAGIHAKQQPDGTFINYPDETRGNLTATVQGYVGMLASGCFHRTEPHMKKAEQFIISHGGLRHVHFMTKWMLAANGLYPWPALYLPLSLMALPPTLPIHFYQFSSYARIHFAPMAVTLNQRFVLINRNISSLHHLDPHMTKNPFTWLRSDAFEERDLTSILLHWKRVFHAPFAFQQLGLQTAKTYMLDRIEKDGTLYSYASATIYMVYSLLSLGVSRYSPIIRRAITGIKSLVTKCNGIPYLENSTSTVWDTALISYALQKNGVTETDGSVTKAADFLLERQHTKIADWSVKNPNSVPGGWGFSNINTNNPDCDDTTAVLKAIPRNHSPAAWERGVSWLLSMQNNDGGFSAFEKNVNHPLIRLLPLESAEDAAVDPSTADLTGRVLHFLGEKVGFTEKHQHIQRAVKWLFEHQEQNGSWYGRWGVCYIYGTWAALTGMHACGVDRKHPGIQKALRWLKSIQNDDGSWGESCKSAEIKTYVPLHRGTIVQTAWALDALLTYENSEHPSVVKGMQYLTDSSSHSADSLAYPAGIGLPKQFYIRYHSYPYVFSLLAVGKYLDSIEKETANET

SEQ ID NO: 105 - protein, 556 aa, Bacillus subtilis
  source 1..556: note = AAB84441.1 squalene-hopene cyclase [Bacillus subtilis]
MGTLQEKVRRFQKKTITELRDRQNADGSWTFCFEGPIMTNSFFILLLTSLDEGENEKELISSLAAGIHAKQQPDGTFINYPDETRGNLTATVQGYVGMLASGCFHRTEPHMKKAEQFIISHGGLRHVHFMTKWMLAANGLYPWPALYLPLSLMALPPTLPIHFYQFSSYARIHFAPMAVTLNQRFVLINRNISSLHHLDPHMTKNPFTWLRSDAFEERDLTSILLHWKRVFHAPFAFQQLGLQTAKTYMLDRIEKDGTLYSYASATIYMVYSLLSLGVSRYSPIIRRAITGIKSLVTKCNGIPYLENSTSTVWDTALISYALQKNGVTETDGSVTKAADFLLERQHTKIADWSVKNPNSVPGGWGFSNINTNNPDCDDTTAVLKAIPRNHSPAAWERGVSWLLSMQNNDGGFSAFEKNVNHPLIRLLPLESAEDAAVDPSTADLTGRVLHFLGEKVGFTEKHQHIQRAVKWLFEHQEQNGSWYGRWGVCYIYGTWAALTGMHACGLTESIPVYKRLCVGSNPYKMMTEAGENPAKAPKSKHMYRFIEEPLYKRPGL

SEQ ID NO: 106 - protein, 631 aa, Bacillus thuringiensis
  source 1..631: note = strain: al hakam; note = ABK86448.1 sporulene cyclase [Bacillus thuringiensis str. Al Hakam]
MNIVIRISKGWVSNLLLDEKAHEEIVRRATALQTMQWQDGTWRFCFEGAPLTDCHTIFLLKLLGRDKEIEPFVERVASLQTNEGTWKLYEDEVGGNLSATIQSYAALLASKKYTKEDANMKRAENFIQERGGVARAHFMTKFLLAIHGEYEYPSLFHVPTPIMFLQNDSPFSIFELSSSARIHLIPMMLCLNKRFRVGKKLLPNLNHIAGGGGEWFREDRSPVFQTLLSDVKQIISYPLSLHHKGYKEIERFMKERIDENGTLYSYATASFYMIYALLALGHSLQSSMIQKAIAGITSYIWKMERGNHLQNSPSTVWDTALLSYALQEAQVSKDNKMIQNATAYLLKKQHTKKADWSVHAQALTPGGWGFSDVNTTIPDIDDTTAVLRALARSRGNKNIDNAWKKGVNWIKGLQNNDGGWGAFEKGVTSKLLAKLPIENASDMITDPSTPDITGRVLEFFGTYAQNELPEKQIQRAINWLMNVQEENGSWYGKWGICYIYGTWAVMTGLRSLGIPSSNPSLTRAASWLEHIQHEDGGWGESCHSSVEKRFVTLPFSTPSQTAWALDALISYYDTETPAIRKGVSYLLSNPYVNERYPTGTGLPGAFYIRYHSYAHIYPLLTLAHYIKKYRK

SEQ ID NO: 107 - protein, 653 aa, Bacillus thuringiensis
  source 1..653: note = serovar: tochigiensis; note = EEM21409.1 Squalene-hopene cyclase [Bacillus thuringiensis serovar tochigiensis BGSC 4Y1]
MSNLLLYEKAHEEIARRATALQTMQREDGTWRFCFEGAPLTDCHMIFLLKLLGRDKEIEPFVKRLATLQTNEGTWKLYEDEVGGNLSATIQSYAALLASGKYTKEDANMKRAENFIKERGGVARAHFMTKFLLAIHGEYEYPSLFHVPTPIMFLQNDSPLSIFELSSSARIHLIPMMLCLNKRFRVGKKLLPNLNHIAGGGGEWFREERSPLFQTLLSDVKQIISYPLSLHHKGYEEVERFMKERIDENGTLYSYATASFYMIYALLALGHSLQSSMIQKAIAGITSYIWKMESGNHVQNSPSTVWDTALLSYALQEAHVPKDNKMLQNATAYLLKKQHTKKADWSVHAPALTPGGWGFSDVNTTVPDVDDTTAVLRVLARSKGNEKLDHAWQKGINWVKGLQNNDGGWGAFEKGVTSRILANLPIENASDMITDPSTPDITGRVLEFFGTYAQNELPEKQKQSAINWLMNAQEENGSWYGKWGICYIYGTWAVMTGLRSFGIPSSNPSLKRAALWLEHIQHKDGGWGESCHSSVEKRFVTLPFSTPSQTAWALDALISYYDTETPVIRKGISYLLANPYVNEKYPTGTGLPGGFYIRYHSYAHIYPLLTLTHYIKNIENKPRDISRFIFLGSRSLLKRIRLCFPYFSVDWRF

SEQ ID NO: 108 - protein, 620 aa, Bacillus thuringiensis
  source 1..620: note = serovar: huazhongensis; note = EEM82653.1 Squalene-hopene cyclase [Bacillus thuringiensis serovar huazhongensis BGSC 4BD1]
MSNLLLYEKVHEEIARRTTALQTMQRQDGTWRFCFEGAPLTDCHMIFLLKLLGRDKEIEPFVKRLASLQTNEGTWKLYEDEAGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLLSDVKKIITYPLSLHHKGYEEVERFMKERIDENGTLYSYATASFYMIYALLALGHSIQSPIIEKAITGITSYIWKMERGSHLQNSPSTIWDTALLSYALQEAQVPKASKVIHNASAYLLRKQQTKKVDWSVHAPDLFPGGWGFSDVNTTIPDIDDTTAALRALARSRGNENVDNAWKRAVNWVKGLQNNDGGWGAFEKGVTSRILANLPIENASDMITDPSTPDITGRVLEFFGTYTQNELPEKQKQSAINWLMNVQEENGSWYGKWGICYIYGTWAVMTGLRSFGIPSSNPSLKRAALWLEHIQHEDGGWGESCQSSVEKRFVTLPFSTPSQTAWALDALISYYDKETPVIRKGISYLLSNSYINEKYPTGTGLPGGFYIRYHSYAHIYPLLTLAHYAKKYKK

SEQ ID NO: 109 - protein, 620 aa, Bacillus thuringiensis
  source 1..620: note = serovar: kurstaki; note = EEM52372.1 Squalene-hopene cyclase [Bacillus thuringiensis serovar kurstaki str. T03a001]
MSNLLLYEKVYEEIARRTTALQTMQRQDGTWRFCFEGAPLTDCHMIFLLKLLGRDKEIEPFVKRLASLQTNEGTWKLYEDEVGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLVSDVKKIITYPLSLHHKGYEEVERFMKGRIDENGTLYSYATASFYMIYALLALGHSIQSPIIEKAIMGITSYIWKMERGSHLQNSPSTIWDTALLSYALQEAQVPKVSKVIQNASAYLLRKQQTKKVDWSVHAPDLFPGGWGFSDVNTTIPDIDDTTAVLRALARSRGNENVDNAWKRAVNWVKGLQNNDGGWGAFEKGVTSRILANLPIENASDMITDPSTPDITGRVLEFFGTYTQNELPEKQKQSAINWLMNVQEENGSWYGKWGICYIYGTWAVLTGLRSLGIPSSDSSLKRAVLWLEHIQHEDGGWGESCQSSVEKRFVTLPFSTPSQTAWALDALISYYDKETPVIRKGISYLLSNPYINEKYPTGTGLPGGFYIRYHSYAHIYPLLTLAHYAKKYRK

SEQ ID NO: 110 - protein, 620 aa, Bacillus thuringiensis
  source 1..620: note = strain: bt407; note = EEM27851.1 Squalene-hopene cyclase [Bacillus thuringiensis Bt407]
MSNLLLYEKVHEEIARRTTALQTMQRQDGTWQFCFEGAPLTDCHMIFLLKLLGRDKEIEPFVKRLASLQTNEGTWKLYEDEMGGNLSATIQSYAALLASEKYTKEDANMKRAENFIKERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLASDVKKIITYPLSLHHKGYEEVERFMKERIDENGTLYSYATASFYMIYALLALGHSIQSPIIEKAIMGITSYIWKMERGSHLQNSPSTIWDTALLSYALQEAQVPKASKVIQNASAYLLRKQQTKKVDWSVHAPDLFPGGWGFSDVNTTIPDIDDTTAVLRALARSRGNENVDNAWKRAVNWVKGLQNNDGGWGAFEKGVTSRILANLPIENASDMITDPSTPDITGRVLEFFGTYGQNELPEKQKQSAINWLTNAQEENGSWYGKWGICYIYGTWAVLTGLRSLGIPSSDPSLKRAALWLEHIQHEDGGWGESCHSSVEKRFVTLPFSTPSQTAWALDALISYYDKETPVIRKGISYLLSNPYINEKYPTGTGLPGGFYICYHSYAHIYPLLTLAHYAKKYRK

SEQ ID NO: 111 - protein, 620 aa, Bacillus thuringiensis
  source 1..620: note = serovar: sotto; note = EEM40716.1 Squalene-hopene cyclase [Bacillus thuringiensis serovar sotto str. T04001]
MSNLLLYEKVHEEIARRTTALQTMQRQDGTWRFCFEGAPLTDCHMIFLLKLLGRDKEIEPFVKRLASLQTNEGTWKLYEDEVGGNLSATIQSYAALLASEKYTKEDANMKRAEMFINERGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLLSEVKKIITYPLSLHHKGYEEVERFMKERIDENGTLYSYATASFYMIYALLALGHSIQSPIIQKAITGIASYIWKMERGSHLQNSPSTVWDTALLSYALQEAQVPKASKVIQNASAYLLRKQQTKKVDWSVHAPNLFPGGWGFSDVNTMIPDIDDTTAVLRALARSRGDENVDNAWKRAVNWVKGLQNNDGGWGAFEKGVTSRILANLPIENASDMITDPSTPDITGRVLEFFGTYAQNELPEKQKQSAINWLMNVQEENGSWYGKWGICYIYGTWAVLTGLRSLGIPSSDPSLKRAALWLEHIQHEDGGWGESCQSSVEKRFVTLPFSTPSQTAWALDALISYYEKETPIIRKGISYLLSNPYVNEKYPTGTGLPGGFYIRYHSYAHIYPLLTLAHYTKKYRK

SEQ ID NO: 112 - protein, 617 aa, Bacillus thuringiensis
  source 1..617: note = serovar: pakistani; note = EEM46814.1 Squalene-hopene cyclase [Bacillus thuringiensis serovar pakistani str. T13001]
MLLYEKVHEEIARRTTALQTMQRQDGTWRFCFEGAPLTDCHMIFLLKLLGKDKEIEPFVKRLASLQTNEGTWKLYEDEVGGNLSATIQSYAALLASEKYTKEDVNMKRAEMFINEHGGVARAHFMTKFLLAIHGEYEYPSLFHLPTPIMFLQNDSPLSIFELSSSARIHLIPMMLCLNKRFRVGKKLLPNLNHIAGGGGEWFREDRSPVFQTLVSDVKKIITYPLSLHHKGYEEVERFMKERIDENGTLYSYATASFYMIYALLALGHSIQSPIIQKAITGITSYIWKMERGSHLQNSPSTVWDTALLSYALQEAQVPKASKVIHNASAYLLRKQQTKKVDWSVHAPDLFPGGWGFSDVNTTIPDIDDTTAALRALARSRGNENVDTAWKRAVNWVKGLQNNDGGWGAFEKGVTSRILANLPIENASDMITDPSTPDITGRVLEFFGTYTQNELPEKQKQSAINWLMNVQEENGSWYGKWGICYIYGTWAVLTGLRSLGIPSSDPSVKRAALWLEHIQHEDGGWGESCQSSVEKRFVTLPFSTPSQTAWALDALISYYDKETPVIRKGISYLLSNSYINEKYPTGTGLPGGFYIRYHSYAHIYPLLTLAHYAKKYRK

SEQ ID NO: 113 - protein, 617 aa, Bacillus thuringiensis
  source 1..617: note = strain: ibl 200; note = EEM94969.1 Squalene-hopene cyclase [Bacillus thuringiensis IBL 200]
MLLYEKVHEEIARRTTALQTMQRQDGTWRFCFEGAPLTDCHMIFLLKLLGRDKEIEPFVKRLASLQTNEGTWKLYEDEVGGNLSATIQSYAALLASEKYTKEDANMKRAEMFINERGGVARAHFMTKFLLAVHGEYEYPSLFHLPTPIMFLQNDSPLSIFELSSSARIHLIPMMLCLNKRFRVGKKLLPNLNHIAGGGGEWFREDRSPVFQTLLSEVKKIITYPLSLHHKGYEAVERFMKERIDENGTLYSYATASFYMIYALLALGHSIQSPIIQKAITGITSYIWKMERGSHLQNSPSTVWDTALLSYALQEAQVPKASKGIQNASAYLLRKQQTKKVDWSVHAPDLFPGGWGFSDVNTTIPDIDDTTAVLRALARSRGNENVDNSWKRAVNWVKGLQNNDGGWGAFEKGVTSRILANLPIENASDMIPDPSTPDITGRVLEFFGTYAQNELPEKQKQSAINWLMNIQEENGSWYGKWGICYIYGTWAVLTGLRSLGIPSSDPSLKRAALWLEHIQHEDGGWGESCQSSVEKRFVTLPFSTPSQTAWALDALISYYEKETPVIRKGISYLLSNPYVNEKYPTGTGLPGGFYIRYHSYTHIYPLLTLAHYAKKYRK

SEQ ID NO: 114 - protein, 617 aa, Bacillus mycoides
  source 1..617: note = strain: kbab4; note = ABY44436.1 squalene/oxidosqualene cyclase [Bacillus mycoides KBAB4]
MLLYDKVHEEIERRTTALQTMQRQDGTWQFCFEGALLTDCHMIFLLKLLGRNDEIEPFVKRLVSLQTNEGTWKLYEDEKGGNLSATIQAYAALLASEKYSKEDMNMRRAEMFIKEHGGVSRAHFMTKFLLAIHGEYEFPALFHFPTPILFLQDDSPLSIFGLSSSARIHLIPMMICMNKRFRVEKKLLPNLNHIAGGGGQWFREERSPLIQSFLGDVKKVISYPLSLHHKGYEEVERFMKERIDENGTLYSYASATFYMIYALLALGHSIQSPIIEKAVTGLKSYIWKMDRGSHLQNSPSTVWDTALLSYSLQEAKVTNENKMIQRATEYLLQKQQTKKVDWSVHASSLVAGGWGFSDVNTTIPDIDDTTAALRALARSRGNDRVDDAWGRGVEWVKGLQNNDGGWGAFERGVTSKLLSNLPIENASDMITDPSTPDITGRVLELFGTYAPNELLEEQKKKAIKWLMDVQEQNGSWYGKWGICYIYGTWATMTGLRALGVPSTHPALKKAASWLEHLQHEDGGWGESCQSSVEKKFISLPFSTPSQTAWALDALISYYDQETPIIRKGISYLLAQSTMNEKYPTGTGLPGGFYIRYHSYGHIYPLLALAHYVKKYRK

SEQ ID NO: 115 - protein, 651 aa, Pedosphaera parvula
  source 1..651: note = strain: ellin514; note = EEF59508.1 squalene-hopene cyclase [Pedosphaera parvula Ellin514]
MKSEEVTIKPAVGLEKDELNAAITRSQSFLLCEQKPEGYWVGELMVDSTIVSDTIAYHHWNGKVDPEWQRKAVNHILSMQLPEGGWNIYQNGPPEVNATIKAYLALKLAGIPITDPRMLKARQVALTLGGVPRMNTFSKLYLALLGLWPWKYVPTIPCEVLLLGKWFHVNIWDMSNWSRAMIVPLAIINHYKPTRPVKVDLSELFLEGFHERDLALPKDPQSFTWRNFFLGLDQLHKFAELWVNAGIHPFRRLALKKCEQWMLERFEGSDGLAAIFPAMLNSLIALKSLGYPDDHPEVLRAERELKKLEHETKDTVRIEPCLSPGWDTAIAAMCLRESGVPAEHPRLKKAGDWLVNREVRFKADWHHKNPVDVEPSGWVFQFNNKWNPDLDDTAMVLLALRLIPTDHPRRRDEAFQRGLKWLLAFQCRDGGWAAYDKDCTKNILEKVPFADHNAMLDPECADITARVLELLGFEGYALDHPQVQEAVEYLREHQETDGSWYGRWGVNYIYGTWQTLRGLWALKMDMNQPWLLKARDWLESVQLPDGGWGERCNTYDDPVFKGQGPSTASQTAWAVMALCTFGDPKRPSLVRGIQYLIENQNEDGSWTELETTGTGFPRVYYLKYDIYRNTWPLLAMATYRKMLDPKEVRVK

SEQ ID NO: 116 - protein, 662 aa, Beijerinckia indica
  source 1..662: note = subspecies: indica; note = ACB96717.1 squalene-hopene cyclase [Beijerinckia indica subsp. indica ATCC 9039]
MPEEAILTETHPLDATTIETAITRARKALLGEQRADGHFVFELEADVSIPCEYILFYHFIGRPAPAELEAKIGHYLRARQSAEHDGWPLFQDGAFNISSSVKAYFALKAIGDTPDMPHMQRARTAILAHGGAAAANVFTRSLLALFGLIPWHGIPVMPIEIMHLPEWFPFHIAKISYWGRTVLVPMMVVHALKPKPANTCTIRIDELFVIPPDQVRHWPGSPGKRFPWTAIFAGIDKVLQIAEPYFPRRSRQSAIDKAVAFVTKRLNGEDGLGAIYPAMAYSALMYLSIGRSLSDPHIQLVLKAIDKLVVVKDHEAYVQPCVSPVWDTALASHALMEAGDGDKPILDSLKKGLAWLKPLQVTDIAGDWAWKKPDVKPGGWAFQYGNAYYPDLDDTAVVVMAMDRARDRWPEIDEDNFRPSIARAREWIVGLQSENGGFGAFDADNDRDYLNAIPFADHGALLDPPTADVTARCISMLTQLGEKPENSETLRRAIAYLFAEQEKDGSWFGRWGLNYIYGTWSVLCSLNAAGIAHDAPEVRRAVAWLRTIQNEDGGWGEDAESYALDYAGYQQAPSTSSQTAWAVLGLMAAGEKDDPAVARGIAYLTRTQGEDGFWTEKRFTATGFPRVFYLRYHGYSKFFPLWAMARYRNLHNGNHASVLTGM

SEQ ID NO: 117 - protein, 691 aa, Blastopirellula marina
  source 1..691: note = strain: dsm 3645; note = EAQ81955.1 squalene-hopene cyclase [Blastopirellula marina DSM 3645]
MEGASPTASNRISQYAVDLRAKARAAVASTCDWLLSHQHADGHWCAELEGDSILQSEYILLLAWLGKERTEIARRCAAHLLKQQEPNGAWTQFPGAPIDVGSSVKAYFALKLTGHDAAADYMVRARNAILEAGGADKVNSFTRFYLALLGQIPFELCPAVPPEMVLLPNWSPINIYRISSWSRTIFVPLSIVWAHRAARDIVEDVSIHELFIRKPEDWPELRCPGLEKPAGLFSWDRFFRTADSGLKLLEKYGLRPLRKRALRQAQQWMLDRFQQSDGPGAIFPPIVWSAIALRTLGYAEDSPEIQYCLDHLERLVLEDGETTKLQPCKSPVWDTSITLRALAAAGLGLAQEPTCRGVEWLLSKEVRVPGDWTNNVDCEPGGWFFEYENAFYPDNDDTSMGIMALADQLAAANITLEVHPGETLANTSVVVGGRGIAEQLAGSSAAMMEQAAAATRRAVAWMVAMQNKDGGWGAFDKNNDAEFLCHVPFADHNAMIDPSTPDLSARVIESFGRLGVTIESPGKLGDTVRRAVAYIRANQLSDGSWFGRWGVNYIYGTWQCLVGLRAVGVPANDPAIEQGKLWLLAHQQACGGWGESCETYEDPSLRGQGSPTASQTAWALLGIIAAGGANLAEVVHGVQYLMDTQREDGAWDEIEFTGTGFPRVFYLKYHYYPIYFPLLALAEWNRATARS

SEQ ID NO: 118 - protein, 610 aa, Blastopirellula marina
  source 1..610: note = strain: dsm 3645; note = EAQ78122.1 probable squalene-hopene cyclase [Blastopirellula marina DSM 3645]
MDQDRLQRCYAIARDDLLAQRNGQGHWTGELSTSALSTATAVSALQLVVRHDPAQSERLMPLIEGGVRYLTEHQNPDGGWGDTDRSYSNIATTMLAVAALTIAERREALFEQLAFAENYIEAQGGIPGLRRRYGKDKTFAVPILTNYALAGLVDWREVSPLPFELACLPQKFYKLVKLPVVSYAIPALVAIGQARYFHRPPFNPLMRGLRGAAVKKSLAVLERMQPASGGYLEAAPLTSFVVMSLASIGNASHPVAQNGVQFLVDSVREDGSWPIDSNLANWVTTLSISALATGGDDIAELDCLPWVLANQYQETHPFTGADPGGWGWTDLSGSVPDADDTPGAMLAIAHFFHSPRADNETRRQIASAAISGARWLLDLQNSDGGWPTFCAGWGTQPFDRSGSDLTAHAIRALHAWRSELGDLPVERAIERGLRYLQKQQRDDGSWLPLWFGNQDIHDDENPIYGTVKVLLAYRDLGKMSSETAQRGAAWLAARQNEDGGFGGGPSISTLCGGPGESSVEETALAIEALFAAENSNISAEIVPPAVGWLCQRVEEGSYVNCTPIGFYFSKLWYYEKLYPRVMTVTSLGAALQANASVPPAPETVTTSSDH

SEQ ID NO: 119 - protein, 660 aa, Bradyrhizobium japonicum
  source 1..660: note = CAA60250.1 squalene-hopene cyclase [Bradyrhizobium japonicum]
MDSVNATAREAKESKISESEILESSIASATQGVLGFQQSDGHWVFELEADCTIPAEYVLLRHYLAEPVDTVLEAKIGNYLRRVQGAHGGWPLVHDGEFDMSASVKAYFALKMIGDSIDAPHMVRAREAIHARGGAIHSNVFTRFMLAMFGIVTWRAVPVLPIEIMLLPFWSPFHINKISYWARTTMVPLMVIAALKPRAKNPKGVGIDELFLQDPRSIGMTAKAPHQSMAWFLLFRSLDAILRVIEPLFPKSLRKRAIDTALAFSEERLNGEDGMGAIYPPMANLVMMYDALGKDENYPPRAVTRRGIDKLLVIGDDEAYCQPCVSPVWDTTLTAHALLEAGGDKAGPAAKHGLDWLIPKQELEVKGDWAVKRPDVRPGGWAFQYNNAYYPDLDDTAVVVMSMDRMRREHGVTGYDSAIDRGREWIEGMQSDDGGWAAFDVNNLEYYLNNIPFSDHGALLDPPTEDVTARCVSMLAQLGETAKTSKHVADGVAYLRKTQHPEGSWYGRWGMNFIYGTWSVLCALNMAGVRHDDPMIRKAADWLASIQNKDGGWGEDTVSYRLDYKGWEAAPSTASQTAWALLALMAAGEVDHPAVARGVEYLIATQNEKGLWDEQRYTATGFPRVFYLRYHGYSKFFPLWGLARYRNLRNTNSRVVGVGM

SEQ ID NO: 120 - protein, 656 aa, Acetobacter pasteurianus; identical to SEQ ID NO: 27
  source 1..656: note = strain: ifo 3283-01; note = BAH98349.1 squalene--hopene cyclase [Acetobacter pasteurianus IFO 3283-01]

SEQ ID NO: 121 - protein, 684 aa, Bradyrhizobium sp.
  source 1..684: note = strain: ors 278; note = CAL79893.1 Squalene-hopene cyclase [Bradyrhizobium sp. ORS 278]
MTLTSSASARAPRDPGNYQTALQSTVRAAADWLIANQKPDGHWVGRAESNACMEAQWCLALWFMGLEDHPLRKRLGQSLLDTQRPDGAWQVYFNAPNGDINATVEAYAALRSLGYPDSEPAVRRAREWIEAKGGLRNIRVFTRYWLALIGEWPWEKTPNIPPEVIWFPLWFPFSIYNFAQWARATLMPIALLSARRPSRPLPPENRLDTLFPRGRDAFDYELPVKANAGGWDKFFRGADKVLHALQNFGNRLNLGLFRPAATSRVLEWMIRHQDFDGAWGGIQPPWIYGLMALYAEGYPLNHPVLAKGLDALNDPGWRVDVGEATYIQATNSPVWDTILTLLAFDDAGVLGDYPDAVDKAVNWVLARQVRVPGDWSMKLPHVKPGGWAFEYANNHYPDTDDTAVALIALAPLRHDPKWKAKGIDEAIQLGVDWLIGMQSQGGGWGAFDKDNNQQILTKIPFCDYGEALDPPSVDVTAHIVEAFGKLGISRNHPSMVQALDYIRKEQEPSGPWFGRWGVNYVYGTGAVLPALAAIGEDMTQPYIGRACDWLVAHQQPDGGWGESCASYMDISAVGRGTTTASQTAWALMALLAANRPQDKDAIERGCMWLVERQSAGTWDEPEFTGTGFPGYGVGQTIKLTDPSLQERLMQGPELSRAFMLRYGMYRHYFPLMALGRALRPQGHG

SEQ ID NO: 122 - protein, 660 aa, Brevibacillus brevis
  source 1..660: note = strain: nbrc 100599; note = BAH44778.1 putative squalene-hopene cyclase [Brevibacillus brevis NBRC 100599]
MRIGTTTNPSMPFPLSSSGAVFYREVNELREVQQEINRIQAFLLQRQQEDGTWRFCLESSPMTDSHMIILLRTLGIHDERLMEKLTAHITALQHDNGAWKLYPDEQEGHLSTTIDSYYALLLSGKYTKNEPRMALARSFILEKGGLTQANMLTKFATALTGQYQWPSHFLVPVEIALLPPSFPVSFYDFVGYARVHLAPMMIVADRNYVKKPDNAPDLSDLYADTPISRGLYPHRFLENFLKEGQSFLATIHDSLQQLPFLPGQLHKLALRRLEQYILARIEPDGTLYNYSTSTFFMIFALLARGFSPKDPLIQKAMQGLTGSVYDYENGAHLQLATSAVWDTALLTFSLQKSGLSPTHPAIQKANRYLLRKQQHTYGDWKIRNPNGKPGGWGFSDYNTMNPDIDDTTAALRSLRLLARTDVTAATAWKRGLEWLLSMQNDDGGWPAFERNTDADFIRHLPIEGADTVSTDPSSADLTGRTLEFLGNYAGRTLTDLHVEKGVRWLLKHQESDGSWYGRWGIAYLYGTWAAITGLMAVGFSPTEPAIQKAVAWLVANQNPDGGWGESCQSDLKKTYVPLGASTPSQTAWAIDALIAVSSKPTAELQRGIRYLLTHNQANDWTTRYPTGGGRPGGTYFAYHSYRWIWPLLALSHYQVKYANT

SEQ ID NO: 123 - protein, 682 aa, Burkholderia ambifaria
  source 1..682: note = strain: iop40-10; note = EDT05097.1 squalene-hopene cyclase [Burkholderia ambifaria IOP40-10]
MIRRMNKPAPSPWSALDTAIARGRDALMRLQQPDGSWCFELESDATITAEYILMMHFMDKIDDARQEKMARYLRAIQRLDTHGGWDLYLDGDPDLSCSVKAYFALKAAGDSEHAPHMVRARDAILKLGGAARSNVFTRILLATFGQVPWRATPFMPIEFVLFPKWVPISMYKVAYWARTTMVPLLVLCSLKARARNPRNIAIPELFVTPPDQERQYFPPARGMRRAFLALDRVVRHVEPLLPKRLRQRAIRHAQAWCAERMNGEDGLGGIFPPIVYSYQMMDVLGYPDDHPLRRDCENALEKLLVTRPDGSMYCQPCLSPVWDTAWSTMALEQARGVAVPEAGAPAGALDELDARIARAYDWLAERQVNDLRGDWIENAPADTQPGGWAFQYANPYYPDIDDSAVITAMLDRRGRTHRNADGSHPYAARVARALDWMRGLQSRNGGFAAFDADCDRMYLNAIPFADHGALLDPPTEDVSGRVLLCFGVTKRADDRASLARAIDYVKRTQQPDGSWWGRWGTNYLYGTWSVLAGLALAGEDPSQPYIARALAWLRARQHADGGWGETNDSYIDPALAGTNAGESTSNCTAWALLAQMAFGDGESESVKRGIAYLQSVQQDDGFWWHRSHNAPGFPRIFYLKYHGYTAYFPLWALARYRRLAGGASSAGAHTVPASTGADAALA

SEQ ID NO: 124 - protein, 682 aa, Burkholderia ambifaria
  source 1..682: note = strain: mex-5; note = EDT37649.1 squalene-hopene cyclase [Burkholderia ambifaria MEX-5]
MIRRMNKPAPSPWSALDAAIARGRDALMRLQQPDGSWCFELESDATITAEYILMMHFMDKIDDARQEKMARYLRAIQRLDTHGGWDLYVDGDPDVSCSVKAYFALKAAGDSEHAPHMVRARDAILALGGAARSNVFTRILLATFGQVPWRAAPFMPIEFVLFPKWVPISMYKVAYWTRTTMVPLLVLCSLKAHARNPRNIAIPELFVTPPDQERHYFPPARGMRRAFLALDRVVRHAEPLLPKRLRQRAIRHAQAWCAERMNGEDGLGGIFPPIVYSYQMMDVLGYPADHPLRRDCENALEKLLVTRPDGSMYCQPCLSPVWDTAWSTMALEQARGVAVHEAGAPASALDELDARIARAYDWLAERQVNDLRGDWIENAPADTQPGGWAFQYANPYYPDIDDSAVVTAMLDRRGRTHRNADGTHPYAARVARALDWMRGLQSRNGGFAAFDADCDRMYLNAIPFADHGALLDPPTEDVSGRVLLCFGVTKRADDRASLARAIDYVKRTQQPDGSWWGRWGTNYLYGTWSVLAGLALAGEDPSQPYIARALAWLRARQHADGGWGETNDSYIDPALAGTNAGESTSNCTAWALLAQMAFGDGESESVKRGIAYLQSVQQDDGFWWHRSHNAPGFTRIFYLKYHGYTAYFPLWALARYRRLAGGASSAGAHAVPASTAADAALA

SEQ ID NO: 125 - protein, 678 aa, Burkholderia ambifaria
  source 1..678: note = strain: mc40-6; note = ACB68303.1 squalene-hopene cyclase [Burkholderia ambifaria MC40-6]
MNKPAPSPWSALDTAIARGRDALMRLQQPDGSWCFELESDATITAEYILMMHFMDKIDDVRQEKMARYLRAIQRLDTHGGWDLYVDGDPDVSCSVKAYFALKAAGDSEHAPHMVRARDAILALGGAARSNVFTRILLATFGQVPWRATPFMPIEFVLFPKWVPISMYKVAYWARTTMVPLLVLCSLKARARNPRNIAIPELFVTPPDEERHYFPPARGMRRAFLALDRVVRHVEPLLPKRLRQRAIRHAQAWCAERMNGEDGLGGIFPPIVYSYQMMDVLGYPDDHPRRRDCENALEKLLVTRTDGSMYCQPCLSPVWDTAWSTMALEQARAVAVPEAGARASALDELDARIARAYDWLAERQVNDLRGDWIENAPADTQPGGWAFQYANPYYPDIDDTAVVTAMLDRRGRTHRNADGSHPYAARVARALDWMRGLQSRNGGFAAFDADCDRMYLNAIPFADHGALLDPPTEDVSGRVLLCFGVTKRAADRASLARAIDYVKRTQQPDGSWWGRWGTNYLYGTWSVLAGLALAGEDPSQPYIARALAWLRARQHADGGWGETNDSYIDPTLAGTNAGESTSNCTAWALLAQMAFGDCESESVRRGIAYLQSVQQDDGFWWHRSHNAPGFPRIFYLKYHGYTAYFPLWALARYRRLASGVSSAGVHAVPASTGADAALA

SEQ ID NO: 126 - protein, 657 aa, Burkholderia ambifaria
  source 1..657: note = strain: mex-5; note = EDT42454.1 squalene-hopene cyclase [Burkholderia ambifaria MEX-5]
MNDLTEMATLSAGTVPAGLDAAVASATDALLAAQNADGHWVYELEADSTIPAEYVLLVHYLGETPNLELEQKIGRYLRRVQQADGGWPLFTDGAPNISASVKAYFALKVIGDDENAEHMQRARRAIHAMGGAEMSNVFTRIQLALYGAIPWRAVPMMPVEIMLLPQWFPFHLSKVSYWARTVIVPLLVLNAKRPIAKNPRGVRIDELFVDPPVNAGLLPRQGHQSPGWFAFFRVVDHVLRAADGLFPSYTRERAIRQAVSFVDERLNGEDGLGAIYPAMANAVMMYDVLGYAEDHPNRAIARKSIEKLLVVHEDEAYCQPCLSPVWDTSLAAHALLETGDARAEEAVIRGLEWLRPLQILDVRGDWISRRPHVRPGGWAFQYANAHYPDVDDTAVVAVAMDRVQKLKHNDTFRDSIALAREWVVGMQSSDGGWGAFEPENTQYYLNNIPFSDHGALLDPPTADVSGRCLSMLAQLGETPLNSEPARRALDYMLKEQEPDGSWYGRWGMNYVYGTWTALCALNAAGLTPDDPRVKRGAQWLLSIQNKDGGWGEDGDSYKLNYRGFEQAPSTASQTAWALLGLMAAGEVNNPAVARGVEYLIAEQKEHGLWDETRFTATGFPRVFYLRYHGYRKFFPLWALARYRNLKRDNATHVTFGL

SEQ ID NO: 127 - protein, 687 aa, Burkholderia cenocepacia
  source 1..687: note = strain: pc184; note = EAY66961.1 Terpene synthase [Burkholderia cenocepacia PC184]
MIRRMNQSAPSSWSALDAAIARGRDALVRLQQPDGSWCFELESDATITAEYILMMHFMDRIDDVRQEKMARYLRANQRLDTHGAWDLYVDGAPDVSCSVKAYFALKAAGDSEHAPHMIRARDAILKLGGAARSNVFTRILLATFGQVPWRAAPFMAVEFVLFPKWVPISMYKVAYWARTTMVPLLVLCSLKARARNPRNVSIRELFVTPPEQERHYFPPARGMRRLFLALDRTVRPIEPLLPKRLRQRAIRHAEAWCAERMNGEDGLGGIFPPIVYSYQMMQVLGYPDDHPLRRDCENALEKLLVTRPDGSMYCQPCLSPVWDTAWSTMALEQARGVAAPETGDTATGAPRDLDGRIARAYDWLATRQVNDLRGDWIENAPADVEPGGWAFQYANPYYPDIDDTALVTAMLDRRGRTHRAADGTHPYASCVSRALDWMRGLQSRNGGFAAFDADCDRMYLNAIPFADHGALLDPPTEDVSGRVLLCFGVTKRAADRASLARAIDYVKRTQQPDGSWWGRWGTNYLYGTWSVLAGLALAGEDKSQPYIARALDWLRARQHADGGWGETNDSYLDPKLAGTNGGESTSNCTAWALLAQMAFGDCESDSVKRGIAYLQSVQQEDGFWWHRSHNAPGFPRIFYLKYHGYTAYFPLWALARYRRLAGAKDAGATRSGASGASATSVTDDALA

SEQ ID NO: 128 - protein, 684 aa, Burkholderia cenocepacia
  source 1..684: note = strain: mc0-3; note = ACA95661.1 squalene-hopene cyclase [Burkholderia cenocepacia MC0-3]
MIRRMNKSAPSPWSALDAAIARGRDALVRLQQPDGSWCFELESDATITAEYILMMHFMDRIDDVRQERMARYLRANQRLDTHGAWDLYVDGAPDVSCSVKAYFALKAAGDSEHAPHMIRARDAILKLGGAARSNVFTRILLATFGQVPWRAAPFMPIEFVLFPKWVPISMYKVAYWARTTMVPLLVLCSLKARARNPRNVSIRELFVTPPEQERHYFLPARGMRRLFLALDRTVRPIEPLLPKRLRQRAIRHAEAWCAERMNGEDGLGGIFPPIVYSYQMMQVLGYPDDHPLRRDCENALEKLLVTRPDGSMYCQPCLSPVWDTAWSTMALEQARGVAAPETGDTASGALRELDERIARAYDWLATRQVNDLRGDWIENAPADVEPGGWAFQYANPYYPDIDDTALVTAMLDRRGRTHRGADGTHPYASRVARALDWMRGLQSRNGGFAAFDADCDRMYLNAIPFADHGALLDPPTEDVSGRVLLCFGVTKRAADRASLAHAIDYVKRTQQPDGSWWGRWGTNYLYGTWSVLAGLALAGEDKSQPYITRALDWLRARQHADGGWGETNDSYIDPKLAGTNDGESTSNCTAWALLAQMAFGDCESDSVKRGIAYLQSVQQEDGFWWHRSHNAPGFPRIFYLKYHGYTAYFPLWALARYRRLAGAKDADATRSPASATPATDNALA

SEQ ID NO: 129 - protein, 679 aa, Burkholderia cenocepacia
  source 1..679: note = strain: j2315; note = CAR57099.1 squalene-hopene cyclase [Burkholderia cenocepacia J2315]
MIRRMNKSAPSPWSTLDTAIARGRDALVRLQQPDGSWCFELESDATITAEYILMMHFMDRIDDVRQEKMARYLRANQRLDTHGAWDLYVDGAPDVSCSVKAYFALKAAGDSEQAPHMIRARDAILKLGGAARSNVFTRILLATFGQVPWRAAPFMPIEFVLFPKWVPISMYKVAYWARTTMVPLLVLCSLKARARNPRNVSIRELFVTPPEQERRYFPPARGMRRLFLALDRAVRHIEPLMPKRLRQRAIRHAQAWCAERMNGEDGLGGIFPPIVYSYQMMQVLGYPDDHPLRRDCENALEKLLVTRPDGSVYCQPCLSPVWDTAWSTMALEQARGVAAPETGETAAGTLRELDERIARAYDWLAARQVNDLRGDWIENVPADVEPGGWAFQYANPYYPDIDDSALVTAMLDRRGRTHRHADGTNPYAPRVARALDWMRGLQSRNGGFAAFDADCDRMYLNAIPFADHGALLDPPTEDVSGRVLLCFGVTKRAEDRASLARCIDYVKRTQQPDGSWWGRWGTNYLYGTWSVLAGLALAGEDKSQPYIARALDWLRARQHADGGWGETNDSYLDPTLAGTNGGESTSNCTAWALLAQMAFGDCESDSVKRGIAYLQSVQQEDGFWWHRSHNAPGFPRIFYLKYHGYTAYFPLWALARYRRLAGAAAAPPAALVAADTALA

SEQ ID NO: 130 - protein, 657 aa, Burkholderia cenocepacia
  source 1..657: note = strain: j2315; note = CAR56694.1 squalene--hopene cyclase [Burkholderia cenocepacia J2315]
MNDLTEMATLSAGAVPAGVDAAVARATDALLAAQQADGHWVYELEADSTIPAEYVLLVHYLGETPNLELEQKIGKYLRRIQQADGGWPLFTDGAPNISASVKAYFALKVIGDDENAEHMQRARRAIHAMGGAEMSNVFTRIQLALYGAIPWRAVPMMPVEIMLLPQWFPFHLSKVSYWARTVIVPLLVLNAKRPLAKNPRGVRIDELFIDPPVNAGLLPRQGHQSPGWFAFFRVVDHALRAVDGLFPSYTRERAIRQAVSFVDERLNGEDGLGAIYPAMANSVMMYAALGYAEDHPNRAIARKSVEKLLVVHDDEAYCQPCLSPVWDTSLAAHALLETGDARAQEAVLRGLEWLRPLQILDVRGDWISRRPNVRPGGWAFQYANAHYPDVDDTAVVVMAMDRAQKLTQSDTYRESMARAREWVVGMQSSDGGWGAFEPENTQYYLNNIPFSDHGALLDPPTADVSGRCLSMLSQLGETPLNSEPARRALDYMLKEQEPDGSWYGRWGMNYVYGTWTALCSLNAAGLTPDDPRMKRGAQWLLSIQNKDGGWGEDGDSYKLNYRGYEQAPSTASQTAWALLGLMAAGEVNNPAVARGVDYLVAQQNEEGLWDETRFTATGFPRVFYLRYHGYRKFFPLWALARYRNLKRANATRVTVGM

SEQ ID NO: 131 - protein, 658 aa, Burkholderia dolosa
  source 1..658: note = strain: au0158; note = EAY71311.1 Squalene cyclase [Burkholderia dolosa AU0158]
MNDLTDMATLSAGAAPAADLDAAVARATDALLAAQNADGHWVYELEADSTIPAEYVLLVHYLGETPNLELERKIGRYLRRIQQADGGWPLFTDGAPNVSASVKAYFALKVIGDDENAEHMQRARRAIHAMGGAEMSNVFTRIQLALYGAIPWRAVPMMPVEIMLLPQWFPFHLSKVSYWARTVIVPLLVLNAKRPLAKNPRGVRIDELFIDPPVNAGLLPRQGHQSAGWFAFFRVVDHVLRAVDGLFPKYTRERAIRQAVSFVDERLNGEDGLGAIYPAMANAVMMYDVLGYAEDHPNRAIARKSIEKLLVVHDDEAYCQPCLSPVWDTSLAAHALLETGDPRAEDAALRGLEWLRPLQILDVRGDWISRRPNVRPGGWAFQYANAHYPDVDDTAVVAMAMDRAQKLRQSDTYRESIARAREWVVGMQSSDGGWGAFEPENTQYYLNNIPFSDHGALLDPPTADVSGRCLSMLSQLGESALTSEPARRALDYMLKEQEPDGSWYGRWGMNYVYGTWTALCALNAAGLGPDDPRVKRAAQWLLSIQNKDGGWGEDGDSYKLNYRGYEQAPSTASQTAWALLGLMAAGEVNNPAVARGIDYLLAEQKEHGLWDEVRFTATGFPRVFYLRYHGYRKFFPLWALARYRNLKRANATRVTVGM

SEQ ID NO: 132 - protein, 776 aa, Burkholderia glumae
  source 1..776: note = strain: bgr1; note = ACR32572.1 Squalene-hopene cyclase [Burkholderia glumae BGR1]
MNDLTNSSAPGARPDDATPSAAGPTPAEAAGGAVAPSRAVQPADTQTAATGAAGAAAAVGATPAELAATAPASSGTPAGASAAPAPSGTPSVDAPAELASAAPAPSGATPAATATAATAPAPARAASIDAPALAAADLDAAITRATDALLAAQQADGHWIYELEADSTIPAEYVLLVHYLGETPNLELERKIARYLRRVQLPGGGWPLFTDGAPDVSASVKAYFALKMIGDDANAEHMVRARNAIHAMGGAEMSNVFTRIQLALFGVVPWFAVPMMPVEIMLLPQWFPFHLSKVSYWARTVTVPLLVLSAKRPLARNPRGVRVDELFVAPPVNAGLLPRAGHQSPAWFACFRLLDGLLRLTDGLFPRYTRERAIRQALQFVDERLNGEDGLGAIYPAMANSVMMYAALGYPEDHPNRATARRAIEKLLVIHDDEAYCQPCLSPVWDTSLAAHALLETGEPRAEAAAIRGLDWLRPLQILDVRGDWISRRPDVRPGGWAFQYANPHYPDVDDTAVVTLAMDRVAKLAQTDAYRDAIARAREWVVGMQSSDGGWGAFEPENTHQYLNSIPFSDHGALLDPPTADVSGRCLSMLAQLGETAANSAPARRALDYLLAEQGADGSWYGRWGMNYIYGTWSALGALNAAGLPFDDPRVKRAAQWLLSIQNPDGGWGEDGDSYKLDYRGYERAASTASQTAWALLGLMAAGEVEHPAVARGIAWLAAQQREHGLWDEARFTATGFPRVFYLRYHGYRKFFPLWALARYRNLRRTGTRRVTVGM

SEQ ID NO: 133 - protein, 676 aa, Burkholderia glumae
  source 1..676: note = strain: bgr1; note = ACR30752.1 Squalene-hopene cyclase [Burkholderia glumae BGR1]
MIRPMKNSDLPLPSLLDAAILRGRDALAQRQSADGSWCFELESDATITAEYILMMHFMGKIDEARQARMARYLRGIQRLATHGAWDLYVDGAPDVSCSVKAYFALKAAGDSEDAPHMARARETILKLGGAAKSNVFTRILLATFGQVPWRATPFMPVEFVLFPKWVPISMYKVAYWARTTMVPLLVLCSLKARAKNPRNVSIRELFVTAPEAERHYFARGGFVRNLFLGIDRALRPLDALIPKALRRRAIRHAEAWCAERMNGEDGMGGIFPPIVYSYQMMDVLGYPEDHPLRRDCENALDKLLVERPDGSVYCQPCLSPVWDTAWSTMALEQARAVPDPRDAPPVSDAQLQRCIAASYEWLAGKQVTQVRGDWVENAPAATPAGGWAFQYENPYYPDIDDSAVVAAMLHRRGRLLARSTGTDPYAQVVARGLDWMRGLQSRNGGFGAFDADCDRLYLNLIPFADHGALLDPPTEDVSGRVLLCLGVTGRDEDKPALARAIEYVKRMQRADGCWWGRWGTNYIYGTWSVLAGLALAGENPSQPYIARAIAWLRACQNADGGWGETNDSYLDPALAGTNGGESASNVTAWALLAQMAFGDWQSESVQRGIRYLLSVQQADGFWWHRSHNAPGFPRIYYLKYHGYTAYFPLWALARYRRLSQAGAARDVTDGAALAAS

SEQ ID NO: 134 - protein, 683 aa, Paraburkholderia graminis
  source 1..683: note = strain: c4d1m; note = EDT12320.1 squalene-hopene cyclase [Paraburkholderia graminis C4D1M]
MNDLSQAHVLGAAMPETAGEAQNAQAAANSAAAAAEASAVLAPSLDAAITRATDAILAAQKPDGHWVYELEADATIPAEYVLLVHYLGETPNVELEQKIARYLRRIQLPNGGWPLFTDGAIDISASVKAYFALKMIGDPVDAEHMVRAREAILAHGGAETVNVFTRILLALFGVVSWRAVPMMPVEITLLPMWFPFHLSKVSYWARTVIVPLLVLNAKRPLARNPRRVRIDELFRGAPVNTGMPARAPHQHVGWFGFFRVVDTVLRAVDGLFPKATRERAVREAVAFVDQRLNGEDGLGAIFPAMANSVMMYDVLGYPADHPNRAIARRSIEKLLVIKDDEAYCQPCLSPVWDTSLAAHALLETGDARAEQAAERGLAWLRPLQILDVRGDWISRRPNVRPGGWAFQYNNAYYPDVDDTAVVAMAMHRSEALTHSGADREAIARAREWVVGMQSSDGGWGAFEPENTQYYLNNIPFSDHGALLDPPTADVSGRCLSMLAQLGEFPQNSEPAQRALDYMLKEQEADGSWYGRWGLNYIYGTWTALCSLNAAGLPHDDPRIRRAAQWLLSIQNEDGGWGEGGESYKLDYRGYERAPSTASQTAWALMGLMAAGEVDHEAVARGIEYLQREQREHGLWDETRFTATGFPRVFYLRYHGYRKFFPLWALARYRHLKRNGLTRVAVGM

SEQ ID NO: 135 - protein, 657 aa, Burkholderia mallei
  source 1..657: note = strain: savp1; note = ABM48844.1 squalene-hopene cyclase [Burkholderia mallei SAVP1]
MNDMTEMHTLDATAAPAGLDAAVARATDALLAAQQADGHWVYELEADSTIPAEYVLLVHYLGEAPNVELEQKIARYLRRIQQPDGGWPLFTDGAPNISASVKAYFALKVIGDDENAEHMQRARRAIHAMGGAEMSNVFTRIQLALYGVVPWYAVPMMPVEIMLLPQWFPFHLSKVSYWARTVIVPLLVLNAKRPVAKNPRGVRIDELFKGAPVSTGLLPKQPHQSAGWFAFFRAVDGVLRLVDGLFPRYTRERAIRQAVAFVDERLNGEDGLGAIYPAMANAVMMYAALGYPEDHPNRAIARRSIEKLLVVGEQEAYCQPCLSPVWDTSLAAHALLETGDARAREAAVRGLDWLVPRQILDVRGDWISRRPHVRPGGWAFQYANAHYPDVDDTAVVAMAMDRVAKLDRTDAYRESIARAREWVVGMQSSDGGWGAFEPENTQYYLNNIPFSDHGALLDPPTADVSGRCLSMLAQFGETSASSEPARRALDYMLKEQEPDGSWYGRWGMNYIYGTWTALCSLNAAGLGHDDPRVKRAAQWLLSIQNADGGWGEDGDSYKLDYRGYERAPSTSSQTAWALLGLMAAGEVDNPAVARGVDYLLGTQREHGLWDETRFTATGFPRVFYLRYHGYRKFFPLWALARYRNLKRANAMRVTVGM

SEQ ID NO: 136 - protein, 683 aa, Burkholderia multivorans
  source 1..683: note = strain: atcc 17616; note = ABX19650.1 squalene-hopene cyclase [Burkholderia multivorans ATCC 17616]
MIRAMNKSALSPWSALDTAIARGRDALARLQQPDGSWCFELESDATITAEYILMMHFMDRIDDALQERMARYLRAIQRLDTHGAWDLYVDGAPDVSCSVKAYFALKAAGDSEHAPHMIRAREAILKLGGAARSNVFTRILLATFGQVPWRATPFMPIEFVLFPKWVPISMYKVAYWARTTMVPLLVLCSLKARARNPRNVAIPELFVTPPDQERHYFPPTRGMRRAFLILDRVVRHVEPLLPKRLRRRAIRHAEAWCAQRMNGEDGLGGIFPPIVYSYQMMDVLGYPEDHPLRRDCENALAKLLVTRPDGSVYCQPCLSPVWDTAWSTMALEQARSVAVPESDESARALDELDARIARAYDWLATRQVNDLRGDWIENAPADTQPGGWAFQYANPYYPDIDDSAVVTAMLDRRGRTHRNADGSHPYAARVARALDWMRALQSRNGGFAAFDADCDRLYLNAIPFADHGALLDPPTEDVSGRVLLCFGVTRRAEDRASLARAIDYVKRTQQPDGSWWGRWGTNYLYGTWSVLAGLTLAGEDPSQPYIARALEWLRAHQHADGGWGETNDSYLDPALAGTNGGESTSNCTAWALLAQMAFGDCASDSVKRGIAYLQSVQQDDGFWWHRSHNAPGFPRIFYLKYHGYTAYFPLWALARYRRLAGAAEARARASSGRAPHAADTALA

SEQ ID NO: 137 - protein, 657 aa, Burkholderia multivorans
  source 1..657: note = strain: atcc 17616; note = ABX16859.1 squalene-hopene cyclase [Burkholderia multivorans ATCC 17616]
MNDLTDMATLSAGTVPAELDAAVARATDALLAAQNADGHWVYELEADSTIPAEYVLLVHYLGETPNLELEQKIGRYLRRIQQADGGWPLFTDGAPNISASVKAYFALKVIGDDENAEHMQRARRAIHAMGGAEMSNVFTRIQLALYGAIPWRAVPMMPVEIMLLPQWFPFHLSKVSYWARTVIVPLLVLNAKRPLAKNPRGVRIDELFIDPPVNAGLLPRQGHQSAGWFAFFRAVDHVLRAVDGLFPAYTRERAIRQAVAFVDERLNGEDGLGAIYPAMANAVMMYDVLGYAEDHPNRAIARKSIEKLLVVHEDEAYCQPCLSPVWDTSLAAHALLETRDPRAEQAAVRGLDWLRPLQILDVRGDWISRRPHVRPGGWAFQYANPHYPDVDDTAVVAMAMDRAQKLNQSDTYRESIARAREWVVGMQSSDGGWGAFEPENTQYYLNNIPFSDHGALLDPPTADVSGRCLSMLSQLGETALNSDAARRALDYMLKEQEPDGSWYGRWGMNYVYGTWTALCALNAAGLGPDDARVKRAAQWLLSIQNKDGGWGEDGDSYKLNYRGYEPAPSTASQTAWALLGLMAAGEVNNPAVKRGIDYLIAEQKEHGLWDEARFTATGFPRVFYLRYHGYRKFFPLWALARYRNLKRDNTTRVTVGI

SEQ ID NO: 138 - protein, 677 aa, Paraburkholderia phymatum
  source 1..677: note = strain: stm815; note = ACC73258.1 squalene-hopene cyclase [Paraburkholderia phymatum STM815]
MNDLSMTQTLGEVLPQTLIDDHAPVAAALATGAAPVDALDAAVTRATEAILAVQKDDGHWVYELEADATIPAEYVLLVHFLGETPNLELEQKIARYLRRIQLPNGGWPLFTDGAMDVSASVKAYFALKMIGDPEDAAHMVRARECILANGGAEAANVFTRILLALFGVVTWYAVPMMPVEIMLLPKWFPFHLSKVSYWARTVIVPLLVLNAKRPVARNPRGVRIDELFRGAPVTTGLLPRSGHQSKSWFAFFRAVDGVLRVTDGLFPKASRERAIKAAVSFVDERLNGVDGLGAIFPAMANSVMMYDVLGYPADHPNRAIARESIEKLLVVHEDEAYCQPCLSPVWDTSLAAHALLETGDARAEEAAERGLAWLRPLQILDVRGDWISRRPDVRPGGWAFQYNNAHYPDVDDTAVVAMAMHRSAAVTNSNVDANAIARAREWVVGMQSSDGGWGAFEPENTQYYLNNIPFSDHGALLDPPTADVSGRCLSMLAQLGEMPATSEPARRAYDYLLKEQEDDGSWYGRWGMNYIYGTWTALCALNAAGISLEDARIKRAAQWLVSIQNADGGWGEDGTSYKLDYRGYEKAPSIPSQTAWALLGLMAAGYVDHPAVARGIDYLQREQRDHGLWDEERFSATGFPRVFYLRYHGYRKYFPLWALARYRNLKRTGEKRVTVGM

SEQ ID NO: 139 - protein, 676 aa, Paraburkholderia phytofirmans
  source 1..676: note = strain: psjn; note = ACD21317.1 squalene-hopene cyclase [Paraburkholderia phytofirmans PsJN]
MNDLSQAQPLDAILPDFADAAPSAPAPAVTGEAPTASLDAAITRATEAILAAQKPDGHWVYELEADATIPAEYVLLVHYLGETPNLELEQKIARYLRRIQLPDGGWPLFTDGALDISASVKAYFALKMIGDPADAEHMVRAREAILAHGGAETVNVFTRILLALFGVVSWRAVPMMPVEIMLLPMWFPFHLSKVSYWARTVIVPLLVLNAKRPVARNPRRVRIDELFRGAPVNTGPRDRAPHQHAGWFRFFSGVDVLLRAVDGLFPKSTRERAVRQAVAFVDERLNGEDGLGAIFPAMANSVMMYDVLGYPADHPNRAIARQSIDKLLVIKDDEAYCQPCLSPVWDTSLAAHALLETGEAHAEQAAERGLAWLRPLQILDVRGDWISRRPNVRPGGWAFQYNNAHYPDVDDTAVVAMAMQRSATVTQSDVDRDAIARAREWVVGMQSSDGGWGAFEPENTQYYLNNIPFSDHGALLDPPTADVSGRCLSMLAQLGELPQNSEPAQRAFDYMLKEQESDGSWYGRWGLNYIYGTWTALCSLNAAGLPHDDPRMKRAAQWLLSIQNEDGGWGEGGESYKLDYHGYERAPSTASQTAWALMGLMAAGEVNHEAVARGVAYLEREQREHGLWDETRFTATGFPRVFYLRYHGYRKFFPLWALARFRHLKRNGLTRVAVGM

SEQ ID NO: 140 - protein, 657 aa, Burkholderia pseudomallei
  source 1..657: note = strain: 576; note = EEC32728.1 squalene-hopene cyclase [Burkholderia pseudomallei 576]
MNDMTEMHTLDATAAPAGLDAAVARATDALLAAQQADGHWVYELEADSTIPAEYVLLVHYLGEAPNVELEQKIARYLRRIQQPDGGWPLFTDGAPNISASVKAYFALKVIGDDENAEHMQRARRAIHAMGGAEMSNVFTRIQLALYGVVPWYAVPMMPVEIMLLPQWFPFHLSKVSYWARTVIVPLLVLNAKRPVAKNPRGVRIDELFKGAPVSTGLLPKQPHQSAGWFAFFRAVDGVLRLVDGLFPRYTRERAIRQAVAFVDERLNGEDGLGAIYPAMANAVMMYAALGYPEDHPNRAIARRSIEKLLVVGEQEAYCQPCLSPVWDTSLAAHALLETGDARAREAAVRGLDWLVPRQILDVRGDWISRRPHVRPGGWAFQYANAHYPDVDDTAVVAMAMDRVAKLDRTDAYRESIARAREWVVGMQSSDGGWGAFEPENTQYYLNNIPFSDHGALLDPPTADVSGRCLSMLAQFGETSASSEPARRALDYMLKEQEPDGSWYGRWGMNYIYGTWTALCSLNAAGLGHDDPRVKRAAQWLLSIQNADGGWGEDGDSYKLDYRGYERAPSTSSQTAWALLGLMAAGAVDNPAVARGVDYLLGTQREHSLWDETRFTATGFPRVFYLRYHGYRKFFPLWALARYRNLKRANATRVTVGM

SEQ ID NO: 141 - protein, 681 aa, Burkholderia sp.
  source 1..681: note = strain: h160; note = EEA03553.1 squalene-hopene cyclase [Burkholderia sp. H160]
MNDLSQTQPLAAVLPEAADAPAVADASATAAPEPVQAASPSALDASITRATDTILAAQKPDGHWVYELEADATIPAEYVLLVHYLGETPNLELEQKIARYLRRIQLPNGGWPLFTDGALDISASVKAYFALKMIGDPVDAEHMVRARDAILAHGGAERANVFTRILLALFGVVSWRAVPMMPVEIMLLPVWFPFHLSKVSYWARTVIVPLLVLNAKRPLARNPRKVRIDELFRAAPVNTGMNERAPHQHAGWFGFFRCVDTVLRAVDGLLPKATRERAIRAAVAFVDERLNGEDGLGAIFPAMANSVMMYDVLGYPADHPHRAIARKSLDKLLVIKDDEAYCQPCLSPVWDTSLAAHALLETGEARAEQAAERGLAWLRPLQILDVRGDWISRRPNVRPGGWAFQYNNAHYPDVDDTAVVAMAMHRSAALTQSDVDREAIARAREWVVGMQSSDGGWGAFEPENTQYYLNNIPFSDHGALLDPPTADVSGRCLSMFAQIGELPQSSEPARRAFDYMLQEQEPDGSWYGRWGLNYIYGTWTALSSLNAAGMPHDDPRMRRAAQWLVSIQNEDGGWGEGGESYKLDYHGYERAPSTASQTAWALLGLMAAGEVNHEAVARGIDYLQREQREHGLWDETRFSATGFPRVFYLRYHGYRKFFPLWALARFRHLKRHGLTRVTVGM

SEQ ID NO: 142 - protein, 678 aa, Burkholderia lata
  source 1..678: note = ABB06563.1 Terpene synthase/Squalene cyclase [Burkholderia lata]
MIRRMTTPTPSPWSALDTAIARGRDALVRLQQPDGSWCFELESDATITAEYILMMHFMDKIDDLRQEKMARYLRANQRLDTHGGWALYVDGDPDVSCSVKAYFALKAAGDSEHAPHMVRARDAILKLGGAARANVFTRILLATFGQVPWRAAPFMPIEFVLFPKWVPISMYKVAYWARTTMVPLLVLCSLKARARNPRNISIRELFVTPPDEERQYFPPARGMRKLFLALDRTVRHVEPLMPKGLRQRAIRHAEAWCAERMNGEDGLGGIFPPIVYCYQMMEVLGYPDDHPLRRDCENALEKLLVTRPDGSMYCQPCLSPVWDTAWSTMALEQARGVAVAEDGEPGDARRALDERITRAYDWLAERQVNDLRGDWIENAPADVQPGGWAFQYANPYYPDIDDTAVVTAMLDRRGRTHANADGTNPYATRVARALDWMRGLQSRNGGFGAFDADCDRLYLNAIPFADHGALLDPPTEDVSGRVLLCFGVTKRADEHASLARCIDYVKRTQQPDGSWWGRWGTNYIYGTWSVLAGLALAGEDKSQPYIARAIEWLRARQHADGGWGETNDSYIDPKLGGTNGGESTSNFTAWALLAQMAFGDCESDSVKRGIAYLQSVQQEDGFWWHRSHNAPGFPRIFYLKYHGYTAYFPLWALARYRRLAGVANKRVSTADKTADAMA

SEQ ID NO: 143 - protein, 657 aa, Burkholderia lata
  source 1..657: note = ABB10136.1 Terpene synthase/Squalene cyclase [Burkholderia lata]
MNDLTEMATLSAGAVPAGVDTAVARATDALLAAQNADGHWVYELEADSTIPAEYVLLVHYLGETPNLELEQKIGKYLRRIQQADGGWPLFTDGAPNISASVKAYFALKVIGDDENAEHMQRARRAIHAMGGAEMSNVFTRIQLALYGAIPWRAVPMMPVEIMLLPQWFPFHLSKVSYWARTVIVPLLVLNAKRPLAKNPRGVRIDELFIDPPVNAGLLPRQGHQSAGWFAFFRVVDHALRAVDGLFPNYTRERAIRQAVSFVDERLNGEDGLGAIYPAMANSVMMYDVLGYAEDHPNRAIARKSIEKLLVVQEDEAYCQPCLSPVWDTSLAANALLETRDARAEDAAIRGLEWLRPLQILDVRGDWISRRPHVRPGGWAFQYANAHYPDVDDTAVVAVAMERAQQLKQNDAYRDSIARAREWVVGMQSSDGGWGAFEPENTQYYLNNIPFSDHGALLDPPTADVSGRCLSMLSQLGETPLNSEPARRALDYMLKEQEPDGSWYGRWGMNYVYGTWTALCSLNAAGLTPDDPRVKRGAQWLLSIQNKDGGWGEDGDSYKLNYRGFEQAPSTASQTAWALLGLMAAGEVNNPAVARGIDYLIAEQNAEGLWDETRFTATGFPRVFYLRYHGYRKFFPLWALARYRNLKRDNTTRVTVGL

SEQ ID NO: 144 - protein, 694 aa, Burkholderia sp.
  source 1..694: note = strain: ccge1002; note = EFA54357.1 squalene-hopene cyclase [Burkholderia sp. CCGE1002]
MNDLSQTQPLDAVLPEAADAASNLAEAAVVANAPAVADALATATPSPMQTAGASPLDVSITRATDAILAAQQPDGHWIYELEADATIPAEYVLLVHYLGETPNLELEQKIARYLRRIQLPNGGWPLFTDGALDISASVKAYFALKMIGDPVDAEHMVRARDAILAHGGAEHANVFTRILLALFGVVSWRAVPMMPVEIMLLPMWFPFHLSKVSYWARTVIVPLLVLNAKRPLARNPRKVRIDELFRGAPVNTGMNERAPHQHAGWFGFFRCVDTVLRAVDGLLPKASRERAIRAAVAFVDERLNGEDGLGAIFPAMANSVMMYDVLGYPADHPNRAIARKSLDKLLVIKEDEAYCQPCLSPVWDTSLVAHALLETREARAEQAAERGLAWLRPLQILDVRGDWISRRPNVRPGGWAFQYNNAHYPDVDDTAVVAMAMHRSAALTKSDVDREAIARAREWVVGMQSSEGGWGAFEPENTQYYLNNIPFSDHAALLDPPTADVSGRCLSMFAQIGELPQNSEPAQRAFDYMLQEQESDGSWYGRWGLNYIYGTWTALCSLNAAGMSHDDPRMRRAVQWLVSIQNEDGGWGEGGESYKLDYRGYERAPSTASQTAWALLGLMAAGEVDHDAVARGIDYLQREQREHGLWDETRFTATGFPRVFYLRYHGYRKFFPLWALARFRHLKRNGLTRVTVGM

SEQ ID NO: 145 - protein, 678 aa, Paraburkholderia xenovorans
  source 1..678: note = strain: lb400; note = ABE35912.1 Terpene synthase [Paraburkholderia xenovorans LB400]
MNDLSQTQPRDAVLPEAAGAVPPASAPAPAAASEAPAASLDTAITRATDAILAAQKPDGHWVYELEADATIPAEYVLLVHYLGETPNVELEQKIARYLRRIQLPDGGWPLFTDGAPDVSASVKAYFALKMIGDPADAEHMVRAREAILANGGAEAVNVFTRILLALFGVVSWRAVPMMPVEIMLLPMWFPFHLSKVSYWARTVIVPLLVLNAKRPLARNPRRVRIDELFRGAPVNTGPRDRAPHQHAGWFRFFSGVDMLLRAVDGLFPKATRERAVRAAVAFVDERLNGEDGLGAIFPAMANSVMMYDVLGYPADHPNRAIARQSIEKLLVIKDDEAYCQPCLSPVWDTSLVAHALLETGEARAEQAAERGLAWLRPLQILDVRGDWISRRPNVRPGGWAFQYNNDYYPDVDDTAVVVMAMHRSAALTHSEVDREAIARAREWVVGMQSSDGGWGAFEPENTQYYLNNIPFSDHGALLDPPTADVSGRCLSMLAQLGELPQGSEPAQRAFAYMLKEQEPDGSWYGRWGLNYIYGTWTALCSLNAAGMPHDDPRMKRAAKWLLSIQNEDGGWGEGGESYKLDYHGYERAPSTASQTAWALMGLMAAGEVNHEAVARGVAYLQREQREHGLWDETRFTATGFPRVFYLRYHGYRKFFPLWALARFRHLKRHGLTRVAVGM

SEQ ID NO: 146 - protein, 657 aa, Candidatus Koribacter
  source 1..657: note = subspecies: versatilis; note = ABF40741.1 Squalene cyclase [Candidatus Koribacter versatilis Ellin345]
MDDRHIQSEITFGKIDGIRERIQQAMDAAKRYLFSKQDPEGFWCGELEADTTLQSDYIVMHTLLGTGDPVKMQKAGKQILQHQNPDGGWNIYPDGPSNISAAVKAYFSLKLIGHKPDEPEMTKAREWILAHGGVTACNTFSKMYLCFFGQYDYDTVPAIPPEIVLFPNWFWFNLYEISSWSRGILVPLAICYAKKPFKKIPDEANIDELFVEGRHANLHLTWDKKPFSWRNFFLVLNNMVHFFERVHVRPLRKLAMKRAEKWMLERLEMSDGLGGIYPAILNSIIALRALGYSTDDPQVIRAMDEFEKLGIEEDDTFRMQPCMSPVWDTAYALYALGEAGVPGSDPRMQKAAEWMLKKQVTHKGDWAVKVRNVQPGGWYFEFNNEFYPDVDDTAQVILSLNHVRTSNERYQDDTVKRALDWQLAMQCKNGGWASFDKDNNKMVFQYIPFADHNAMLDPATVDITGRVLEALSHHGYSLKDKVVQRAVKFIQSEQEPDGSWFGRWGVNYIYGTMLCLRGLAAVGVDHHEPMVQQAAEWLRMVQNPDGGWGESVGSYDDPKLRGQGPSTASQTAWAVMGLLAANDLRSDSVTRGIAWLLENQKPNGSWWEKWITGTGFPRVFYLKYTMYAEYFPLIAFAEYLRRLNTPLDEKVKLGPQA

SEQ ID NO: 147 - protein, 488 aa, Candidatus Kuenenia
  source 1..488: note = subspecies: stuttgartiensis; note = CAJ71215.1 strongly similar to squalene-hopene cyclase (C-terminus) [Candidatus Kuenenia stuttgartiensis]
MILFPAGFYFSIYEISYWSRCIVVPLSIAIARKPHVTVGDDLLKELYLVPREDVVYRIERDQDGFCWYNFFIDADSIFRRYEQHPIKFIRRIAKKMAEKWLLEHMEKSGGLGAIWPAMINSIFAMKCLDYPDDHPALTAQMKEVEALVIYEGDMLYLQPCVSPVWDTAWSIIAMNDSGIPGSHPVLQKAGKWLLSKEVRDFGDWKLKCKVEEPSGWYFQYANEFYPDTDDTGAVLMALQRVSLPEDMHKEKTLLRALRWLQAMQCDDGGWGAFDRNNNKTILNNIPFADFNALLDPSTSDVTGRCIEFFGRIGFNKTYLNIKKAVEFLKKEQDEDGSWFGRWGSNYIYGTWSVISGLIAVGEDINKAYIKKAIAWLKSVQNSDGGWGETIKSYEDSALKGIGKSTPSQTAWALLTLITAGEIKSSSTERGIDFLLSTQKEDGSWDEREFTATGFPKVFYLKYHMYRNYFPLMALGRYRHFTHKLATSQ

SEQ ID NO: 148 - protein, 641 aa, Candidatus Solibacter
  source 1..641: note = subspecies: usitatus; note = ABJ82180.1 squalene-hopene cyclase [Candidatus Solibacter usitatus Ellin6076]
MSYEWTEPVRPGRRHAVSPVQNFCQSLAPAIQRACDALFSQQAADGFWCGELTADTTLESDYILLQLWLNQPDDHGWNPPTRPRIDRAGRSILERQLPDGGFNIYAGGPSEVSATIKAYCALKLAGLDPHSPPLRRARERILALGGLQAANSYVKINLSLFGLYPRKHVPSVPPEIVMLPGNVLYEMSSWTRSILVPLSIVQARGSNRRAPNGFNLDELLLPGVKLALPKRKGLAVLFHHLDRMFKVWEKRGSERIRGAAIREAERWLIARTHYTEGLGAIYPAMMYFIMALDALGYAEDHPDRSEAIRHFESLLIETDDRFLFQPCVSPVWDTAICAFALGEAGNTDDPRMTLAADWLISKEVRRKGDWSIKRPDTEPSGWAFEFANEFYPDIDDTAMVLLALMHANGSNPEAQAAAERRAVNWLLAMQSSDGGWAAFDVDNNWAMLNQVPFADHNAMLDPTCPDITGRVLECLCRRGMAGHDAARRGVAYLLQAQEKDGSWYGRWGVNYIYGSFLAMRGLTTSGAPGSQDAVDRAARWLRAIQNPDGGWGESCASYARDGYVAAPSSASQTAWALLGLCAAGDRDSAQFRRGVEYLLTLQAPDGKWPEGATTGTGFPNVFYLTYAMYRDYFPLLALSQV

SEQ ID NO: 149 - protein, 641 aa, Candidatus Solibacter
  source 1..641: note = subspecies: usitatus; note = ABJ82254.1 squalene-hopene cyclase [Candidatus Solibacter usitatus Ellin6076]
MSAPSHVGNTLEHAAELATRKAMAYLTCLQERDGHWCAELTADTTLESDYILFQLWLYPPQDGKWEPETRPLIRKAVNSILERQLPDGGFNICVGGPSEVSASVKAYVAMKLAGLPPEDDRMARLRERILALGGIQAANSYVKVNLSLFDLYPREFSPSIPPEVALLPFDLLYQMSAWTRAIVISLGIVHAANPRRPAPAGFNLQELWLPGVSPEFRRDPSFFTWHNTFLTVDKALKLWERYGSKAVRRRAVEKAKTWMIERLHHSDGLGAIYPPMMYSVMALDVLGYAKDDPLRVEALRHFNNLMVDDGDRFFFQPCFSPVWDTAIGAYALVQADPSHEAIAPAADWLIAKEVRRKGDWSVKRPNTEPSGWAFEYSNEYYPDIDDTAMVMLALGETRASNTEAQAAACKRGLAWLLAMQSSDGGWAAFDADNNWEFLSQVPFADHNAMLDPTCADITGRVLEALASQGLDRNHKAVRRGAEWLIRHQENDGSWYGRWGVAYIYGTCFALRGLAASGENDREAHILRAGEWLRSIQNADGGWGESCKSYDNRIFTGGPSTPSQTAWAILGLIAGGDANSLSVQHGIEYLLETQRSDGSWDEQFATGTGFPRVFYLNYHMYKDYFPLLALASFVKARAGSNG

SEQ ID NO: 150 - protein, 644 aa, Catenulispora acidiphila
  source 1..644: note = strain: dsm 44928; note = ACU75510.1 squalene-hopene cyclase [Catenulispora acidiphila DSM 44928]
MTDVIDKAVAATGPADPSQGAAATLQAAADHLLGLQDDAGWWKGELETNVTMDAEDLLLRQFLGIRTEEVTREAGDWIRSQQRADGTWANFFDGPADLSTTIEAYTALRMAGDAKDAEHMRAARTYILDSGGIEASRVFTRIWLALFGEWQWSDLPVMPPELIYLPKWFPLNVYDWACWARQTVVPLTIVNALRPVRPLGFDLKELRTGRRAPAQRGLFSTLDRALHVYERKPLRSVRDAALRRSADWIIARQEADGSWGGIQPPWVYSLMALNLLGYGVDHPVMRKGIEGLDRFTIRDERGRRLEACQSPVWDTVLAMTALRDAELPENHPALVKAADWVLGEEITNPGDWSVRRPRVAPGGWAFEFDNDGYPDVDDTAEVVLALNRVAHPDAPAAIRRGVDWLEGMACKDGGYGAFDADNTRTLALKLPFCDFGAVIDPPTADVTAHTLEAYAALGLANSRASQRALEWLVKAQERDGSWFGRWGANHVYGTGAVVPAMVAVGVDPEDEMIRRAVRWLEEHQNDDGGWGEDLRSYRDKSWIGRGVSTASQTAWALLALLAAGEERGTAVEQGVRFLIRTQRADGTWDEDHYTGTGFPGDFYLNYHLYRLVFPISALGRYVRAVGAAGDGGDAGHAGHAGTVS

SEQ ID NO: 151 - protein, 657 aa, Chthoniobacter flavus
  source 1..657: note = strain: ellin428; note = EDY15838.1 squalene-hopene cyclase [Chthoniobacter flavus Ellin428]
MIFTDTPTGSTQNRLDVAIRRAQQNLLRLQHNEGYWCGELFVDSTLCSDYVLFMHWADEIDPVMEEKCVAHIRRRQLEDGGWNIYEGGPSDVNATVKAYFALKLAGHAPTQPWMQEARACILRLGGIPKMNTYAKLYLALLGQFPWRYLPTVPVEIMFMPRWFFFDIYEVSSWSRAMLMPLAILNHYKPTKHLPADKQLHELYPIGSEESDLGLGMQKPRFSWPNFFLFCDRLIKIMHSLPWKPWKRAALARAEAWMTQRMGEGSDGLAAIFPAMLNSMIALRTLRYSREHPLYVKAKNDFAGLFVDDPQDFRIQPCLSPVWDTAINLVALLESGLDPHDPKIEAAVNWLKEKEVRINGDWYVKNHHVPPSGWAFEFNNVYYPDTDDTMMVLAALARAGAHEESAPVETKAMFERALKWLLSFQCRDGGWAAFDKDVTQGWLEDVPFADHNAILDPTCSDLTGRVLELLGLIDYDRNCTPVRRALKFLRDTQEDDGSWYGRWGVNYIYGTWQVLRGLRSIGEDMRQQWIVRARDWLESCQNEDGGWGETCASYDDPTLKGKGPSTASQTAWALMGLIAAADPTEPGAFDRKSIRQGVDYLLSTQVADGSWVEPEVTGTGFPRVFYLRYDMYRNNFPLMALATYRKAREGKLPVRQRE

SEQ ID NO: 152 - protein, 603 aa, Chthoniobacter flavus
  source 1..603: note = strain: ellin428; note = EDY22035.1 putative squalene-hopene cyclase [Chthoniobacter flavus Ellin428]
METLSRSRLEAALAKATQALLTELNPAGHWSGELSSSALSTATAIVALGAVDREQQRELIAGGMRWLAQHQNADGGWGDTVKSRSNISTTALCWAAVSTSTEHAESAAKAEAWLTRAAGSMAQLVPAIEARYGKDRTFSVPILMHLAICGRVSWSQIPALPFELAALPHQLFGALQLPVVSYALPALIAIGQAIHHHAPPTNPLLNGLRKSARARTLEVLESIQPQNGGFLEATPLTSFVTMALASAGEAQHPVARRGVSFLQASVQRDGSWAIDTNLATWVTTLSIKALAHQPGALSPERALTLREWLLGQQYVVEHPYTHAAPGGWAWTDLPGGVPDADDTPGALLALLHLGVVDAPTRQAGQIGVRWLLDLQNRDGGIPTFCRGWGALPFDRSSPDLTAHTLRAWTAWLPQLDESLKRRTLRAVTKAIHFLATHQRTDGSWLPLWFGNEHAPDDENPLYGTAKVVIALRELLNRDFTLPNGMLERALCWLVERQDISGGWSGAKNGPVSVEETALAVEALAGTGHVSATDRGAAWLTEQIEADTWREPAPIGFYFAKLWYYERLYPQIWTVGALGRVAALRVGESESDTPAGLHRATSET

SEQ ID NO: 153 - protein, 645 aa, Crocosphaera watsonii
  source 1..645: note = strain: wh 8501; note = EAM53094.1 Terpene synthase [Crocosphaera watsonii WH 8501]
MQTQDRLTQKQPLSLKDAITASQNYLLSLQYPQGYWWAELESNITLTAETVLLHKIWGTDKTRPLHKVEAYLRQQQREHGGWELFYGDGGEISTSVEAYMALRLLGVPQDDPALIRAKDFIISKGGISKTRIFTKFHLALIGCYDWKGIPSIPPWIMLFPDSFPFTIYEMASWARESTVPLIIVFNDKPVFSVDPVFNLDELYAEGVENVKYELPKNNNWGDIFLGIDQVFKFAEQVDLVPFRKEGLKAAEKWILNHQQETGDWGGIMPPMLNSLLAFRTLNYDVNDPSVKLGFEAIDRFSIEEDDTYRLQACVSPIWDTAWCVRALTDSGLEKDHFSLVKAGKWLLDKQVMEYGDWAVKNKAGKPGGWAFEFTNRFYPDLDDSATVVMALNGIKLPDEARKQAAINRCLQWIETMQCKEGGWAAFDLNNDQAWLNEVPYGDLKAMIDPNTADVTARVVEMLGSCDLEIESDRLNKSLNYLYKEQEKDGSWFGRWGVNYIYGTSGVLSALAVINPEKHKTQMEQGINWLLSCQNKDGGWGETCRSYNDPSLKGKGVSTPSQTAWSLIGLLDAGEALNKFETDAIERGVNYLLDTQTEEGTWEESEFTGTGFPCHFYIRYHFYRHYFPLIALGRYQNLSSEFGVRS

SEQ ID NO: 154 - protein, 681 aa, Cupriavidus taiwanensis
  source 1..681: note = strain: lmg 19424; note = CAQ72562.1 Squalene hopene cyclase [Cupriavidus taiwanensis LMG 19424]
MNETAFANPAPQVGPAQRQPAAPQEAPAARLPAPALDRGIDRALDALLHQQRPDGHWVYELEADATIPAEYVLMVHYLGEDPDRDLEARIARYLRRIQNPDGGWPLFHQGRSDISASVKAYFALKMAGDDPQSAPMQRARQAIHAMGGAEATNVFTRTLLALYGVLPWKAVPMMPVEIMLLPRWFPFHLSKVSYWARTVIVPLLVLNSLRPQARNPRGVGINELFVGNCHTVGLPPRAAHQHAGWYTVFRGLDALLRLAEPLFPRTLRRRAIAAAQRFVRERLNGEDGLGAIFPAMANSVMMFDVLGVPPEDPARAVARRSIERLLVEHGDEAYCQPCLSPVWDTALATHALLETGEARAAQAAGRALDWLRPLQVLDLRGDWAVRRPLVRPGGWAFQYANAYYPDVDDTAVVAAAMDRFMRAHHAPGRYGEAVARATEWIVGMQSGNGGWGAFEPENTHLYLNNIPFADHGALLDPPTADVSARCLSMLCQTGATPANSEPAARALRYLLAEQMPDGSWFGRWGTNYIYGTWSALCALNAAGLPPEAPELCRAVAWLARIQNADGGWGEDGSSYRLDYSGYEPAPSVASQTAWALLALMAAGAAQHPAVARGIDYLLRTQQPGGLWHEPRFTAVGFPRVFYLRYHGYARYFPLWALARYRNLQRGLGDHGGNSGQVAWGL

SEQ ID NO: 155 - protein, 663 aa, Crocosphaera subtropica
  source 1..663: note = strain: atcc 51142; note = ACB53858.1 squalene-hopene-cyclase [Crocosphaera subtropica ATCC 51142]
MLTYKEYRRSVTEIAMQTRDRQTQKPALSLNDAITASQNYLLSLQYPQGYWWAELESNITLTAETVLLHKIWGTDKTRPLHKVEAYLRQQQREQGGWELFYGDGGEISTSVEAYMALRLLGVPQDDPALIRAKDFILSKGGISKTRIFTKFHLALIGCYSWKGIPSIPPWIMLFPNSFPFTIYEMASWARESTVPLIIVFNDKPVFAVDPIFNLDELYAEGIENVKYELPKNNNWGDIFLGLDKVFKFAEQVDLVPFRKKGLQAAERWMLNHQQETGDWGGIMPPMVNSLLAFRVLNYDVNDPSVQRGFEAIDRFSIEENETYRVQACVSPVWDTAWCVRALTNSGLPKDHFSLVKAGKWLLEKQCLEYGDWAVKNKTGKPGGWAFEFTNRFYPDIDDSAVVVMALNGIKLPDEARKQAAINRCVKWIETMQCKEGGWAAFDVDNDQAWLNEVPYGDLKAMIDPNTADVTARVVEMVGSCDLEISSKRLNKALNYLYKEQEKDGSWFGRWGVNYIYGTSGVLSALAVINPEKHQPQIEQGINWLLSCQNKDGGWGETCWSYNDSNLKGKGISTASQTAWALIGLLDAGEALNHFETDSIQRGISYLLNTQTEEGTWEESEFTGTGFPCHFYIRYHFYRHYFPLIALGRYQNLSSEFGIRNSEL

SEQ ID NO: 156 - protein, 643 aa, Gloeothece citriformis
  source 1..643: note = strain: pcc 7424; note = ACK71719.1 squalene/oxidosqualene cyclase [Gloeothece citriformis PCC 7424]
MQIQDRNSSPQVTEVLNQVKDAIAASQDYLMSIQYPEGYWWAELESNVTITAEVVLLHKIWGTDKTRPLHKVETYLRRQQREHGGWELFYGDGGDLSTSVEAYMALRLLGVSIDDPALIRGREFILKRGGISKSRIFTKLHLALIGCYDWRGIPSIPPWIMLLPENFPFTIYEMSSWARSSTVPLLIVFDKKPVYCCDPTINLDELYSEGIENVKYDLPKTGDWTDIFVWLDGVFKFAQDYNLVPLRQESLQAAERWVLERQEDSGDWGGIIPAMLNSLLALRALNYEAVDPIVHRGLQSVDNFAIETEDTYHVQPCISPVWDTAWAIRALVESGLKADDPRLVKGAQWLLDKQILDYGDWAVKNKQGTPGGWAFEFDNRWYPDLDDSAVVVMALDQVKMPNEDLKNGAIRRCVRWMATMQCKDGGWGAFDLDNDQNWLNFLPYADLKAMIDPNTSDVTARVLEMLGTCGLIMDSNRVQKAIAYLEKEQEPDGSWFGRWGVNYIYGTSGVLSALAVIAPETHQKELKKGAAWLVGCQNADGGWGETCFSYNDSSLKGKGDSTASQTAWGLIGLLAAGEATGEFFKTAIERGVNYLLKTQREDGTWDENYFTGTGFPCHFYLKYHLYLQYFPLIALSRYQRLLT

SEQ ID NO: 157 - protein, 651 aa, Cyanothece sp.
  source 1..651: note = strain: pcc 8802; note = EDY02410.1 squalene/oxidosqualene cyclase [Cyanothece sp. PCC 8802]
MQTQNRVTSTQKVELSNLTQAIIASQNYILSRQYPEGYWWGELESNITLTAETVLLHKIWKTDKTRPFHKVETYLRRQQNEQGGWELFYGDGGELSTSVEAYMALRLLGVTPEDPALIRAKDFILSKGGISKTRIFTKFHLALIGCYDWKGIPSIPPWIMLFPDNFPFTIYEMSSWARESTVPLLIVFDKKPIFEIEPAFNLDELYAEGVENVKYALPRNHNWSDIFLGLDKLFKWTEKNNLVPFHKKSLQAAEKWMLNHQQESGDWGGIMPPMVNSLIAFKVLNYDVADPSVQRGFEAIDRFSIEEEDTYRVQACVSPVWDTAWVIRALVDSGLKPDHPSLVKAGEWLLDKQILEYGDWAIKNKQGKPGGWAFEFINRFYPDLDDSAVVVMALNGIKLPDENRKKAAINRCLEWMATMQCKPGGWAAFDVDNDQAWINEIPYGDLKAMIDPNTADVTARVLEMVGSCGLKMDENRVQKALFYLEKEQESDGSWFGRWGVNYIYGTSGVLSALAVIAPNTHKPQMEKAVNWLISCQNEDGGWGETCWSYNDSSLKGTGISTASQTAWAIIGLLDAGEALETLATDAIKRGIDYLLATQTPDGTWEEAEFTGTGFPCHFYIRYHLYRHYFPLIALGRYWKIGLKTPSVIPLN

SEQ ID NO: 158 - protein, 647 aa, Rippkaea orientalis
  source 1..647: note = strain: pcc 8801; note = ACK66841.1 squalene/oxidosqualene cyclase [Rippkaea orientalis PCC 8801]
MQTQNRVTSTQKVELSNLTKAIIASQNYIMSRQYPEGYWWGELESNITLTAETILLHKIWKTDKTRPFHKVETYLRRQQNEQGGWELFYGDGGELSTSVEAYMALRLLGVTPEDPALIRAKDFILSQGGISKTRIFTKFHLALIGCYDWKGIPSIPPWIMLFPDNFPFTIYEMSSWARESTVPLLIVFDKKPIFEIEPAFNLDELYAEGVENVKYALPRNHNWSDIFLGLDKLFKWTEKNNLVPFHKKSLQAAERWMLNHQQESGDWGGIMPPMVNSLIAFKVLNYDVADPSVQRGFEAIDRFSIEEEDTYRVQACVSPVWDTAWVIRALVDSGLKPDHPSLVKAGEWLLDKQILEYGDWAIKNKQGKPGGWAFEFINRFYPDLDDSAVVVMALNGIKLPDENCKKAAINRCLEWMATMQCKPGGWAAFDVDNDQAWINEIPYGDLKAMIDPNTADVTARVLEMVGSCGLKMDENRVQKALFYLEKEQESDGSWFGRWGVNYIYGTSGVLSALAVIAPNTHKPQMEKAVNWLISCQNEDGGWGETCWSYNDPSLKGTGVSTASQTAWALIGLLDAGEALETLATDAIKRGINYLLDTQTPDGTWEEAEFTGTGFPCHFYIRYHLYRHYFPLIALGRYWKIGLKNLKG

SEQ ID NO: 159 - protein, 643 aa, Cyanothece sp.
  source 1..643: note = strain: pcc 7822; note = EDX97382.1 squalene/oxidosqualene cyclase [Cyanothece sp. PCC 7822]
MQIQARNISTKVTEVFSKVKEAIAASQQYLLSIQYPEGYWWAELESNVTITAEAVLLHKIWGTDTTRPLHKVETYLRRQQREHGGWELFYGDGGDLSTSVEAYMALRLLGVSASDPALVRAKAFILSRGGISKSRIFTKMHLALIGCYDWRGVPSIPPWIMLLPENFPFTIYEMSSWARGSTVPLLIVFDKKPVYQCGITLDELYSEGINHVRYDLPRNGDWTDVFVWLDGVFKFAETNNLIPFRNESLKAAERWVLERQEDTGDWGGIIPAMLNSLLALRALDYEVNDPIVHRGFKSVDNFAIETEETYHVQPCISPVWDTAWVLRALVESGLKPDEPVLVKGAQWLLDKQILDYGDWAVKNKEGTPGGWAFEFDNRWYPDLDDSAVVVMALEQVKMPDEQLKYGAMRRCVRWMATMQCKAGGWGAFDVNNDQNWLNYLPYADLKAMIDPNTADVTARVLEMLGTCELSMDHDRVKRAIAYLEQEQEADGSWFGRWGVNYIYGTSGALSALAAIAPVTHQAQIEKGAAWLVGCQNPDGGWGETCFSYNNPALRGKGDSTASQTAWGLIGLLAAGEATGKFAKTALERGVNYLLATQRPDGTWDESYFTGTGFPCHFYLKYHLYLQYFPLIALSRYQRLLGFN

SEQ ID NO: 160 - protein, 640 aa, Crocosphaera chwakensis
  source 1..640: note = strain: ccy0110; note = EAZ91809.1 squalene-hopene-cyclase [Crocosphaera chwakensis CCY0110]
MQTRDRQTHKPALSLNDAITASQNYLLSLQYPQGYWWAELESNITLTAETVLLHKIWGTDKTRPLHKVEAYLRQQQREHGGWELFYGDGGEISTSVEAYMALRLLGVPSNDPALIRAKNFIISQGGISKTRIFTKFHLALIGCYSWKGIPSIPPWIMLFPNSFPFTIYEMASWARESTVPLIIVFNDKPVFAIDPIFNLDELYAEGIENVKYELPKNNNWGDLFLGLDKVFKLAEQVDLVPFRKQGLQAAERWMLDHQQETGDWGGIMPPMVNSLLAFRVLNYDVADPSVQRGFEAIDRFSIEENDTYRVQACVSPVWDTAWCIRALTDSGLPKDHFSLVKAGKWLLEKQVLEYGDWAVKNKTGKPGGWAFEFTNRFYPDIDDSATVVMALNGIKLPDEALKQAAINRCLKWIETMQCKAGGWAAFDVDNDQAWLNEIPYGDLKAMIDPNTADVTARVVEMVGSCDLEMSSDRLNKALDYLYEEQEKDGSWFGRWGVNYIYGTSGVLSALAVINPKQHKSQIEQGMNWLLSCQNEDGGWGETCWSYNDLSLKGKGVSTPSQTAWALIGLLDAGEVLNHFETDSIERGINYLLNTQTEEGTWEESEFTGTGFPCHFYIRYHFYRHYFPLIALGRYQQMLGS

SEQ ID NO: 161 - protein, 668 aa, Cyanothece sp.
  source 1..668: note = strain: pcc 7425; note = ACL45896.1 squalene/oxidosqualene cyclase [Cyanothece sp. PCC 7425]
MQIRATVDTAKLEKAIAASQEHLLSTQYPEGYWWAELESNVTMTAEVVLLHKIWKTDGTRPMHKAEKYLRSEQREHGGWELFYGDGGDLSTSVETYTALRLLGVPASDPALLKAKDFILRRGGISKTRIFTKLHLALIGCYDWRGLPSLPPWVMLLPENFPFTIYELSSWARGSTVPLLIVMDRKPVFSVNPQINVDELYAEGRDRVKFELPRKGDWTDLFIELDGLFKFTEQNNLVPFREEGLRAAERWVLERQEATGDWGGIIPAMLNSLLALRALGYHPADPYVRRGMAAVDRFAIETADTYRVQPCVSPVWDTALVMRGLIDSGLPADHPAIVKAGEWLLEKQILAYGDWAVKNKTGQPGAWAFEFENRFYPDVDDSAVVVMALQAAQLPDEDLKQQAIERCVKWIATMQCKPGGWAAFDVDNDQDWLNQIPYGDLKAMIDPNTADVTARVLEMIGRSGVTTGEASVERALAYLRREQEVEGCWFGRWGVNYIYGTSGVLAALALIAPKSDHAMIQRGADWLVRCQNADGGWGETCRSYNDPHLKGQGPSTASQTAWALIGLLAAGEATGEFAWGAIDRGINYLLATQQQDGRWDEDWFTGTGFPGHFYLKYHLYQQHFPLTALGRYSSLTGLKQELKIPLQLKSKPEVVMIEDSDLLSDEDAT

SEQ ID NO: 162 - protein, 651 aa, Rippkaea orientalis; identical to SEQ ID NO: 157
  source 1..651: note = strain: pcc 8802; note = ACV02092.1 squalene-hopene cyclase [Rippkaea orientalis PCC 8802]

SEQ ID NO: 163 - protein, 705 aa, Desulfovibrio salexigens
  source 1..705: note = strain: dsm 2638; note = EEC62384.1 squalene-hopene cyclase [Desulfovibrio salexigens DSM 2638]
MNKKSAMKLKKKAKNHVVSLLQPTDALNRVMKRFRSLQSPEGYWVFALEADVTIPSEYIMFNRFLGRKMDKGLAERLGNYIRAKQMADGGWPLHDNDGPVNISASVKAYMALKMLGDNKDAEHMVRARQIILAKGGAETANVFTRICLATFGQIPWHCPPAMPIEIVLLPKWFFFHLDKVSYWSRSVIYPLLIIYAKQPVCRLRPEEAVPELFCKPAEEHIHIDKYRDKGWRKNLFILLDRVLKRTIHLVPKSINKKALNYAEKWTREHMAGRGGIGAIFPAMANAVMALSLLGYDESDPDFARGMQSVDDLMVDKFHVPEKSPWEHTVITGGAELSAAPELDISPDHGTAENLEQAMCQPCNSPIWDTCLTLSAMMEAGENQDSKSTQQALNWLWDQQIFFRGDWISKAPKLEGGGWAFQFENTFYPDLDDTAMVLMAMCRAGVLDQPEHRENFIKGVNWLIGMQSSNGGWAAFDIDNCAEYLNDIPFADHGALLDPPTSDLTARVIELLGVLGYDKSFRPIKDGIEFLKKEQEDDGSWFGRWGVNYIYGTWSVLCGLRQAGEDMNSSYVCKAVEWFENHQNKDGGWGESCLSYNDKNYAGLGDSTASQTAWALLGLMAAGRVHSKAVSRGVRYLLDTQKDDGSWDESLFTGTGFPRVFYLRYHGYSQYFPMWALGVYQRFSADEDTKQIMMRRKSPLDLGRKW

SEQ ID NO: 164 - protein, 685 aa, Dryopteris crassirhizoma
  source 1..685: note = BAG68223.1 dammaradiene synthase [Dryopteris crassirhizoma]
  VARIANT 145: note = X may be any amino acid.
MLPYNQDFYNEDEALKDDHCEGAGNVSNPPTLDEAIKRSQDFLLSQQYPEGYWWAELEGNPTITSHTVILYKILGIEDEYPMDKMEKYLRRMQCIHGGWELFYGDGGQLSVTIESYVALRLLNVPPTDPALKKALKFIIDKGGVXKSRMFTKICLALLGCFDWRGIPSLPPWVMLLPGWFLSSIYETACWARGCVVPLIVVFDKKPVFKVSPEVSFDELYAEGREHACKTLPFCGDWTSHFFIAVDRVFKMMERLGVVPFQQWGIREAEKWLLERQEDTGDFLGVYPPMFYSVVCMKTLGYEVTDPVVRRALLSFKKFSIERADECSVQSSLSPVWDTALVVRSLVESGLPPDHPALQRAGEWLLQKQITKHGDWSFKNQSGVAGGWAFQFFNRWYPDLDDSAVVVMALDCLKLPNEDVKNGAITRCLKWISSMQCKGGGWAAFDKDNHQHWINSTPFSDLKAMVDPSTTDISARVLEMVGRLKLHGTSFDEAHFLPPESIARGLVYLRREQENEGCWFGRWGVNYIYGTCGALVALSLVAPMTHEEEIARGARWLVQVQNMHGKKINGPQDGGWGETCFSYNDPALKGQGDVSTASQTAWALQGLLAAGDALGKYEVESIGHGVQYLLSTQRKDGSWHESQFTGGGFPIHFYLRYHFYAQHFTLSSLARYRTRLQASKIKPPIP

SEQ ID NO: 165 - protein, 747 aa, Frankia alni
  source 1..747: note = strain: acn14a; note = CAJ61140.1 squalene-hopene cyclase [Frankia alni ACN14a]
MPDLELRDVDRADGRHHAPNLGRTDTLSPSAPTGEPAPASTPAAVATPTPTPTTAPAPAPAPENALRETVQRAAEHLLRLQDPRGWWKFDLETNPTMDAEDLLLREYLGIRTVEQTEATAKHIRSRRLDDGSWPTYFGGPGELSTTVECYIALRLAGDSPDDEPLRRSAAWIRERGGIPATRVFTRIWLALFGWWRWEDLPVLPPEIMFLPPRAPLSIYSFASWARQTIVPLTIVSAARPQCPAPFDLAELDPDEVPAAQSHGAAQSPDTRSPAGGRTLRGAMRRLGGDRPNTAKVFFRGLDAALHRYHRHPIGPLRRHALRTAERWIIARQEADGCFGGIQPPAVYSIIALRLLGYDLDHPVLAAALRSLDAYTLHREDGSRMIEASQSPIWDTALAVLALADAGIDAPADVDVAPALPTQRVATGAPAPSAPVPTALERAADWLLGQEIQHRRGDWAITHPGVAPGGWAFEFDNDTYPDTDDTAEVVLALHRLNRLRRLRHPTNTRIDAALERSTAWLFALQSRDGGWGAYDSDNASTLVYQIPFADFGALTDPSSADVTAHVVELLCETGRIRDPRTLRGVDWLLRNQEADGSWYGRWGVNYVYGTGSVLPALQAAGLPPTHPAMVAGARWLLSRQNSDGGWGEDIRSYGDPAWSGRGLSTPSQTAWAMLGLLATDHGGVHADALAAAARWLTEQQRPDGGWDEEMFTGTGFPGFFYLNYHGYRLVWPVMALGRYLHSRQHPSD

SEQ ID NO: 166 - protein, 704 aa, Frankia alni
  source 1..704: note = strain: acn14a; note = CAJ60090.1 Squalene--hopene cyclase [Frankia alni ACN14a]
MSLTSDPSPAAPTAEKSPKRPTIPVPATADAYGISRSSPPLPAATGRPQAAGPASAGVATARARDHLLALQSEEGWWKGDLETNVTMDAEDLFMKQFLGIRGDDETEQTARWIRSQQLADGGWPTFYGGPADLSTTIEAYIALRLAGDAVDAPHMARAAELVRAQGGVAASRVFTRIWLAALGQWSWDDVPVIPPELIFLPSWIPLNVYDFACWARQTIVALTIVGSLRPSHDLGFSIDELKVPAAARKPAALRSWEGAFERLDKLLHRYEKRPIKLLRTLALRRATEWVVARQEADGCWGGIQPPWVYSVMALHLMGYPLNHPVIATAFRGMERYVIRRDTPQGPIRQIEACQSPVWDTALAVVALADAGVPGDHPAMVKAGRWLVDEEVRVAGDWAVRRPELAPGGWAFEFDNDFYPDVDDTAEVVLALRRLLGAGHVAPPASRQGRAEAPPVNTVEDADPRLAAAMRAAAARGVDWSVGMRSSNGAWGAFDADNVRTLTTKIPFCDFGEVVDPPSADVTAHIVEMLADLGRSDHPITQRAVQWLLDNQEPGGSWFGRWGVNHLYGTGAVVPALIGAGVPTDHPAITAAVRWLLEHQSPEGGWGEDLRSYTDPAWIGRGELTASQTAWALLALLAVDPHSLAVKRGVRWLCETQRPDGTWDEPYFTGTGFPGDFSLNYHLYRLVFPLTALGRYVSLTGVATP

SEQ ID NO: 167 - protein, 741 aa, Frankia casuarinae
  source 1..741: note = ABD10207.1 Squalene cyclase [Frankia casuarinae]
MSLTSDPSPAAPKAAKSSKRVNIPAPATPDAYGISRSSPPLSGGGVSGGGVSGGGAATADGTPPTTQTSVDPDLAAAMTAANQARDHLLGLQSEEGWWKGDLETNVTIDAEHLFMKQFLGIRTEEETEPIARWVRSQQLADGGWATYYGGPAELSTTVEAYIALRLAGDEPDAPHMAAAAALIRSQGGVAAARVFTRIWLATFGEWSWDDVPVLPPELIFLPSWFPLNVYDFGCWARQTIVALTIVGSLRPVRDLGFSIDEIKVAAPVTPPKPAPLHSWEGAFERLDAILHRYERRPIKVLRTLALRRATEWVVARQEADGCWGGIQPPWIYSVMALHLMGYPLNHPVIATAFRGMERYIIRRETPEGPTAQIEACQSPVWDTALAVVALSDAGVPADHPAMVRAGRWLVDEEVRVAGDWAVRRPALAPGGWAFEFDNDFYPDTDDTAEVVLALRRLLGGSHVTPGGTVTPSGSVTPGGTAELSPAARDRASRGLAAVDPQLAGAMRAAAARGVDWSVGMRSSDGAWGAFDADNVRTLTAKIPFCDFGEVVDPPSADVTAHIVEMLADLGRSDHPITRRAVQWLLDNQEPGGSWFGRWGINHVYGTGAVVPALIAAGVPADHPAITAAVRWLLEHQSPDGGWGEDPRSYDDPAWIGRGELTASQTAWALLALLAVDPHSKAVKRGVRWLCETQRPDGTWDEPQFTGTGFPGDFYLNYHLYRLVFPLTALGRYVTLTGVATP

SEQ ID NO: 168 - protein, 728 aa, Frankia sp.
  source 1..728: note = strain: ean1pec; note = ABW15063.1 squalene-hopene cyclase [Frankia sp. EAN1pec]
MSLTSDQSSAAPTAAAQSPKIPNPSVARPSADAGSFETAGAVRTDSVSIDSVSTGTPVDPVVGAMRRGRDHLLSLQAEEGWWKGELETNVTMDAEDLMLRQFLGILTPSTATETGRWIRSQQLSDGGWATFYGGPSDLSTTIEAYVALRLAGDDPDAPHMRSAAEWVRSAGGIAASRVFTRIWLALFGEWSWDDVPVLPAEMTFLPPWFPLNIYDFACWARQTVVALTIVGSLRPVRSFGFTLDELRVQAPKATKAPLRSWAGAFERLDSVLHRYEKRPFQPLRRLALRRAAEWVIARQEADGCWGGIQPPMVYSIMALHLMGYPLNHPVISMAFRALDRFTIREETPEGTVRRIEACQSPVWDTALAVVALADAGLGGDHPAMVRAGRWLADEEVRVAGDWAVRRPTLAPGGWAFEFDNDFYPDVDDTAEVVIAIRRLLGDGHGPVDHSDGSGPGSAAATAASAAAEAAVAAAGTIAAADPELAARLRAAAERGVDWSVGMRSSNGAWAAFDADNVRTLVRKIPFCDFGEVVDPPSADVTAHMVEMLALLGRSDHPITQRGVRWLLDNQEAGGSWFGRWGVNHVYGTGAVVPALISAGVDAEHPAIVSSMHWLVEHQTPEGGWGEDLRSYRDDEWIGRGEPTASQTAWALLALLAAEPASGTAEWEAVERGVRWLCDTQRPDGTWDEPQFTGTGFPWDFSINYHLYRLVFPVTALGRYVTLTGRSTS

SEQ ID NO: 169 - protein, 689 aa, Frankia sp.
  source 1..689: note = strain: ean1pec; note = ABW14125.1 squalene-hopene cyclase [Frankia sp. EAN1pec]
MFQGSDRPPVTLVMNDMRGPDMNVSDTVSVTRESIPTQTSAGDATARDLTAAVGSELTRALRLATDHLLALQDGTGWWKFDLETNTSMDAEDLLLREYLGIRTTEVTAASARFIRSRQSDDGSWPQYFGGPGELSTTVESYIALRLAGDDASAPHMLSAATWVRDHGGVPATRVFTRIWLALFGWWRWEDLPALPPEIMLLPRRAPLNIYSFGSWARQTLVSLTVVSALRPVRPAPFDLDELYPDGPASAWSGAGPSNVLERISTRFTAKEIFLGIDRLLHVYHRRPVRSMRNHALRAAERWIIARQEADGCFGGIQPPAVYSIIALRLLGYELDHPVLKAALRALDDYSVTLPDGSRMVEASQSPVWDTALAVNALADAGATAAIAPDHPALVRAAGWLLGQEVRHRRGDWAVNHPDVPASGWAFEFENDTYPDTDDTAEVLLALRRVRHPARDELDAAERRAVAWLFGLQSSDGGWGAYDADNTSTIPYQIPFADFGALTDPPSADVTAHVVELLAEAGLGGDDRTRRGVDWLLDHQEADGSWFGRWGVNYVYGTGSVMPALRAAGLEPSHPAMRAGADWLLTHQNADGGWGEDLRSYTDPEWSGRGESTASQTAWAMLALLTVGDQPEVSGALARGARWLADHQRPDGSWDEDQFTGTGFPGDFYINYHGYRLLWPIMALGRYLRG

SEQ ID NO: 170 - protein, 773 aa, Frankia sp.
  source 1..773: note = strain: eui1c; note = EFA59873.1 squalene-hopene cyclase [Frankia sp. EuI1c]
MTTTDETALAAGTPKAAFAPAPRGAADDLVARTVAVEAPPSPAPASDDTLARAVAHLKSLQDEAGWWKGDLETNTTMDSEDLMLRHWLGIWNPEQAERTARFIRSKQYADGSWPIYHAGPGDLNATVESYVALRMVGDSPQDPHMRAAAAWARARGGVPATRIFTRIWLALFGWWRWEDLPVLPPELIFVPAKMPLSIYKFASWGRQTIVAIMVLMAHRPAGTPPFPIAELFPPPATKKKAAAQRKAQKKAGHAGGPTAWRDSSIDDMFTEPAPGTDTLRQPAALAIGPARPAPAKGRRGKGQPAAPDVMGRAKDGGGPGLPLPARLVSRVGFRTRRALRQAALDHVNWNLLFGGIDRFLHVYHRHPIRPVRSLALGLAERWIVVRQEADGCFGGIQPPTVYSIMALRVLGYPMDHPVMTAALRSLDEYSVTLPDGARMQEACQSPVWDTCLATIALADAGVPRDDPSLVRAADWMLAEEVRERRGDWSVPIPDVPTGGWSFEFDNDTYPDVDDSAEVMLALMRVAHPRPEKVVAATYRGLQWVFGMQCADGGWGAFDVDNAGELVYKIPFADFGMLTDPPSADVTAHVVELLGELGLGDDPRTKRGVEWLLHSQEADGSWYGRWGVNHLYGTGGVVPALRAAGLPASHPAIQRAADWLVAKQNPDGGWGESCYSYDEMSTAGVGVSTASQTAWALLALIAAGRVGDGVTGEAAARGVAWLAETQTAEGTWDEDYFTGTGFAGYFYINYHLYRLVWPVMALGRYQAALAGKGH

SEQ ID NO: 171 - protein, 764 aa, Frankia sp.
  source 1..764: note = strain: eui1c; note = EFA59089.1 squalene-hopene cyclase [Frankia sp. EuI1c]
MSLTSDPSPATPATQPTSARPGSLSDRRSRSGGSAVAGPVLVTTRPVAPVAKSGAVTPTATSGAVTSTATSGPALLPDLATDLADPTGPLAGAASATVRAAGGAGTRTQQTGQLGSTELAGPQADQVADRAAAVLGRARDHLLGLQSEAGWWKGELETNVTMDAEDLMLRQFLGILPPELAAETGRWIRSKQQDDGGWPTFHGGPSDLSTTFEAYVGLRLAGDLPDAPHMLAAASFVRAHGGLAATRVFTRIWMALFGEWPWDEVPVLPPELVLLPSWVPLNVYDFGCWARQTVVALTIVGHFRPVRSLGFSIDELRVAAVRPDRAPLVSWTGVFQRLDAGLRRYQRHPVKTLRELALRRATEWVLARQEADGGWGGIQPPWVYSIMALHLMGYSMDHPVLVAALDGLETFTVREQVREGDEVVTVRRLEACQSPVWDTALAVVALADAGLDARHPAMRKAGEWLVREEVTVPGDWRVRRPNLEPGGWAFEFANDIYPDVDDTAEVVLAVRRLLGSGWDDVDPTFAKQARASVERAVNWSVGMRSANGAWGAFDADNVRELATKIPFCDFGEVIDPPSADVTAHMVEMLADLGRADHPVTQRAVRWLLDDQEPGGSWFGRWGVNHVYGTGAVVPALISAGVAADHPAIRSAVRWLVAHQHPDGGWGEDLRSYQDDAWVGRGEPTASQTAWALLALLAADPMNEAVGRGVRWLCDTQLPNGTWDEPYYTGTGFPWDFSINYHLYRLVFPLTALGRYVTLTGRSAA

SEQ ID NO: 172 - protein, 618 aa, Geobacillus sp.
  source 1..618: note = strain: y412mc61; note = EED61885.1 squalene/oxidosqualene cyclase [Geobacillus sp. Y412MC61]
MVADERSALIDALKRSQSVDGSWRFPFETGISTDAYMIILLRTLGIHDEPLIQALVERIESRQDANGAWKLFADEGDGNVTATVEAYYALLYSGYRKKTDSHMQKAKARILEVGGLERVHLFTKVMLALTGQHSWPRRFPLPLVFFLLPPSFPLNMYDLSVYGRANMVPLLVVAERRYSRKTDNSPDLSDLAASRNDWRLPDTEALWSYVKRSLTGLPAWLHRAAEQRAVRYMLEHIEPDGTLYSYFSSTFLLIFALLALGYPKDDPHIARAVRGLRSLRTEIDGHTHMQYTTASVWNTALASYALQEAGVPPTDRTIEKANRYLLSRQHIRYGDWAVHNPYGVPGGWGFSDVNTMNPDVDDTTAALRAIRRAAAKETAFRHAWDRANRWLFSMQNDDGGFAAFEKNVGKRFWRYLPIEGAEFLLMDPSTADLTGRTLEYFGTFAGLTKDHSAIARAIDWLLDHQEADGSWYGRWGICYVYGTWAAVTGLSAVGVPIDHPAMQKAVRWLLSIQNDDGGWGESCKSDGAKTYVPLGASTPVHTAWALDALIAAAERPTPEMKAGVRALVRMLHHPDWTASYPVGQGMAGAFYIHYHGYRYIFPLLALAHYEQKFGPFVD

SEQ ID NO: 173 - protein, 617 aa, Geobacillus thermodenitrificans
  source 1..617: note = EDY05760.1 squalene/oxidosqualene cyclase [Geobacillus thermodenitrificans]
MAGERSALITALKRSQAADGSWRFPFETGISTDAYMIILLRTLDINDEPLIQALVERIESRQEANGAWKLFADEGDGNVTATVEAYYALLYSGYRQPTDRHMQKAKRRILDMGGLDRVHLFTKVMLALTGQYPWPGRFPLPLEFFLLPPSFPLNMYDLSVYGRANMIPLLIAADSRYSRKTDKSPDLSDLFASRGDWGMPESRSLLTYVKRSLIGLPAQLHQAAKQRAVRYLFEHIEPDGTLYSYFSSTFLFIFALLALGYRNDDPRIRQAVRGLRSLRTTIDGHVHLQYTTASVWNTALASYTLQEAGVPMTDRAIEKANRYLLSRQNVRYGDWAVHNPYSTPGGWGFSDVNTMNPDVDDTTAALRAIRQAAAKETAFRHAWDRANQWLFSMQNDDGGFAAFEKNVSSRFWRYLPIEGAEFLLMDPSTADLTGRTLEYFGTFAGLTKDQRAVSRAVDWLLSHQERNGSWYGRWGICYIYGTWAAITGLTAVGVPAHHPALQKAVRWLLSIQNDDGGWGESCKSDGAKTYVPLGDSTPVHTAWALDALVAAAERPTLEMKAGFRALFRLLHHPDWTASYPVGQGMAGAFYIHYHSYRYIFPLLALAHYEQKFGPLDD

SEQ ID NO: 174 - protein, 618 aa, Geobacillus sp.; identical to SEQ ID NO: 172
  source 1..618: note = strain: y412mc52; note = EEN95021.1 squalene/oxidosqualene cyclase [Geobacillus sp. Y412MC52]

SEQ ID NO: 175 - protein, 618 aa, Geobacillus sp.; identical to SEQ ID NO: 172
  source 1..618: note = strain: y412mc61; note = ACX79399.1 squalene/oxidosqualene cyclase [Geobacillus sp. Y412MC61]

SEQ ID NO: 176 - protein, 680 aa, Citrifermentans bemidjiense
  source 1..680: note = strain: bem; note = ACH40355.1 squalene cyclase [Citrifermentans bemidjiense Bem]
MTSPFKHPISNALTSFNGNVAEPEQSVEQQSGAKVHHLPASIWKRKMGRAKSPLDVAIEGSRDFFFQEQLPKGYWWAELESNVTITAEYIMLFHFLGLVDPERQRKMSTYLLSKQTEEGFWTIYYGGPGDLSTTIEAYFALKLSGYPEDHPALAKARAFILEQGGVVKSRVFTKIFLALFGEFDWQGIPSMPVELNLLPDWAYINIYEFSSWARATIVPLSVVMHSRPVRRVPPSARVQELFVRQPTAADYSFAKNDGLFTWEKFFLGLDRVLKVYEKSPLRPFKKTALAKAEEWVLEHQEPTGDWGGIQPAMLNAILALNVLGYRNDHPAVEQGLRALANFCIETEDQLVLQSCVSPVWDTALALKALLDAGVPPDHPSLVKGAQWLLDKEVTRAGDWRVKSPNLEAGGWAFEFLNDWYPDVDDSGFVMIALKGIQVKDHKAMDAAIKRGINWCLGMQSKNGGWGAFDKDNTKHVLNKIPFADLEALIDPPTADLTGRMLELMGTFDYPVTFPAAQRAIEFLKKNQEPEGPWWGRWGVNYLYGTWSVLCGLAAIGEDMDQPYIRKAVNWIKSRQNIDGGWGETCQSYHDRTLAGVGESTPSQTGWALLSLLAAGEMHSATVVRGVQYLISTQNSDGTWDEQQYTGTGFPKYFMIKYHIYRNCFPLMALGTYRTLTRTQP

SEQ ID NO: 177 - protein, 684 aa, Trichlorobacter lovleyi
  source 1..684: note = strain: sz; note = ACD95949.1 squalene-hopene cyclase [Trichlorobacter lovleyi SZ]
MKSRKYPISHALTSFNHTTVAPVEAPAPISVKSPAKVHRLPSSIWKKMEGSAGNPLDKAVELTRDFFFREQLPDGYWWAELESNVTITAEYIMLFHFLGMVDKDKERKMANYLLRQQTEEGYWTVWHNGPGDLSTTIEAYFALKLAGYHADHIALRKARDFILANGGILKSRVFTKTFLAMFGEFSWLGVPSMPIELMLLPDWAYLNVYEFSSWARATIIPMSVLMANRPVYKLPPHARVQELYVRPPRPTDYTFTKEDGIFSLKNFFIGVDHLLKIYESSPIRPFKKRATEKVEQWILEHQEKTGDWGGIQPAMLNAILALHCLGYANDHPAVAKGLEALANFTIEDSDSLVLQSCISPVWDTALVLQAMQEASVPLDHPSLIKASQWLLDREVRIKGDWKIKSPDLEPGGWAFEFQNDWYPDVDDSTAVMIAIKDIKVKNTKARQDAIRRGIDWCLGMQSENGGWAAFDKDNTKHMLNKIPFADLEALIDPPTADLTGRMLELMGNFGYTKDHPQAVSALEFLKNEQEPEGPWFGRWGVNYIYGTWYVLIGLEAIGEDMNSPYIKKSVNWIKSRQNLDGGWGEVCDSYWDRTLMGCGPSTASQTSWALMALMAAGEVGCQAVERGIQYLLATQNSDGTWDEEAFTGTGFPKYFMIKYHIYRNCFPLTALGRYRRLTAGTHAQ

SEQ ID NO: 178 - protein, 730 aa, Geobacter metallireducens
  source 1..730: note = strain: gs-15; note = ABB30662.1 squalene cyclase [Geobacter metallireducens GS-15]
MAKGILNKFAVIAGNKNAGLTAEEECTVVAPIKEVSGKAVHCRQAVKMAEEYLLALQNPEGYWVFELEADVTIPSEYIMLQRFLGREISPELRMRLENYLLDRQLPDGGWPLYAVDGFANISATVKAYLALKVLGHSPQAPHMIRARIMVLSLGGAARCNVFTRILLALFGQLPWHTPPAMPVEIVLLPQRFFFHLSKVSYWSRTVIVPMLLLYAKQPVCRLRPEEGIPELFNTPPDKLRNLDGFQSGRWRKNAFIIIDRLLKRFNRFIPSAIHRKAMAEAEHWTRSRMQGSGGIGAIFPAMAYAVMALRVLGCREDDPDYVRGMQAIDDLLQHRTPQEADSPRTGGPCIDSGTSAAFAFDPSPHAAADGRGNSSICQPCNSPIWDTCLSLSALMEAGMPASHPAAKQAVEWLLSQQIFSPGDWSLKAPDLEGGGWAFQFENTLYPDLDDTSKVIMSLLRAGALENGLYRDRVARGVNWVLGMQSSDGGWAAFDIDNNYHYLNDIPFADHGALLDPSTSDLTGRCIELLSMVGFDRTFPPIAQGIGFLRSKQEGSGAWFGRWGVNYIYGTWSVLSGLRQAGEDMQQPYIRRAVGWLTSCQNHDGGWGETCYSYDDPSLAGQGESTPSQTAWSLLGLMAAGDVHSLAVRRGVRYLLDHQNQWGTWEEKHFTGTGFPRVFYLRYHGYRHYFPLWALGVYSRLSSGQKTRQEERRHSSPGDLHLPWLERIGRR

SEQ ID NO: 179 - protein, 679 aa, Geobacter metallireducens
  source 1..679: note = strain: gs-15; note = ABB33038.1 squalene cyclase [Geobacter metallireducens GS-15]
MKISKHPISHALTSFNETAKETKEEPQKKRGGKVHHLPASIWKKRDVETTSPLDQAIKRSQEFFLREQLPAGYWWAELESNVTITAEYVILFHFMGLVNRDKDRKMATYLLSKQTEEGCWCIWHGGPGDLSTTIEAYFALKLAGYPADHPAMQKARTFILGKGGILKARVFTKIFLALFGEFSWLGVPSMPIEMMLLPNGFTFNLYEFSSWSRATIIPLSIVMAERPVRKLPPWARVQELYVRPPRPMDYTFTKEDGILTWKNIFIGIDHILKVYEASPIRPGMKKAMAIAEQWVLDHQEPTGDWGGIQPAMLNSVLALHCLGYANDHPAVAKGLQALANFCIESDDEIVLQSCISPVWDTALALMAMVDSEVPTDHPALVKAAQWLLDREVRKVGDWKIKAPNLEPGGWAFEFQNDWYPDVDDSGIVMMAIKDVKVKDSKAKAEAIQRGIAWCIGMQSKNGGWGAFDKDNTKHILNKIPFADLEALIDPPTADLTGRMLELMGTFGYPKDHPAAVRALQFVKENQEPDGPWWGRWGVNYIYGTWSVLCGLKAYGEDMGQPYVRKAVEWLAAHQNPDGGWGECCESYCDQKLAGTGPSTASQTGWALLSMLAAGDVDHPAVARGIRYLIETQQPDGTWDEDQFTGTGFPKYFMIKYHIYRNCFPLMAMGRYRALKGHKG

SEQ ID NO: 180 - protein, 714 aa, Geotalea daltonii
  source 1..714: note = strain: frc-32; note = ACM21577.1 squalene cyclase [Geotalea daltonii FRC-32]
MNKHKGTFSVIEGGKTTQARGSETCAIMDAADLEKVTSVAASQLAGQQQDDGHWVFDLEADVTIPAEYVMLQRFIGREIDPEISERLAAYMQERQLPDGGWPLYAVDGNVNISASVKAYFALKLLGHDKNAPHMVRARQLILSLGGAAKCNVFTRITLATFGQIPWHTAPAMPIEIMLLPRWFFFHLNKVAYWSRTVIVPLLILYATQPICRLQYNEGITELFTTPPDMLVHLDKFRHHAWRKNVFIALDRVLKRTMHLVPGRIKQHALAEAERWTRARMQGDGGIGAIYPAMANAVMALKTLGCSDDDADYLRGLEAVDNLMVHRNLKTGTIPMDDDSGGIAIDNSSAAPELSPTYLTDTAGNTEFSFCQPCNSPIWDTCMSLSALCESGYAENNSGVTDRAIKWLFSQQIATPGDWSEKCPGLESGGWAFQYENSRYPDVDDTAKVLMSLFRAGALEKPEYREKIERAIRWVQGMQSTDGGWGAFDVDNDYFYLNDIPFADHGALLDPSTADLTGRCIEMMGMLGHGPDYPPIARGIAYLKKEQEPFGGWFGRWGVNYIYGTWSVLSGLHQAGENMDAPYVRKAVEWLISCQNSDGGWGETCASYDDPSLAGSGASTASQTSWALMALMAAGEWRHSAVRNGVRYLTESYCNGWNEKQFTGTGFPRVFYLRYHGYSLFFPVWALAVYSRYINGTATVQEKVREKQFRQCLMV

SEQ ID NO: 181 - protein, 680 aa, Geobacter sp.
  source 1..680: note = strain: m21; note = EDV72707.1 squalene-hopene cyclase [Geobacter sp. M21]
MTSPFKHPISNALTSFNGNFAEPEQCVEQQTGAKVHHLPASIWKRKMGKAKSPLDVAIEGSRDFFFQEQLPKGYWWAELESNVTITAEYIMLFHFLGLVDRERQRKMSNYLLSKQTEEGFWPIYYGGPGDLSTTIEAYFALKLSGYPADHPALAKARAFILEQGGVVKSRVFTKIFLALFGEFEWQGVPSMPVELNLLPDWAYINIYEFSSWARATIVPLSVVMHSRPVRRVPPSARVQELFVRQPTAADYSFAKNDGIFTWENFFLGLDRVLKVYEKSPLRPFKNMALAKAEEWVLEHQEPTGDWGGIQPAMLNAVLALNVLGYQNDHPAVEQGLRALANFCIETEDQLVLQSCVSPVWDTALALKALLDAGVPPDHPSLVKGAQWLLDKEVTRPGDWRVKSPALEPGGWAFEFLNDWYPDVDDSGFVMIALKGIQVKDRKSMDAAIKRGINWCLGMQSKNGGWGAFDKDNTRHVLNKIPFADLEALIDPPTADLTGRMLELMGTFNYPITLPAAQRAIEFLKKNQEPEGPWWGRWGVNYLYGTWSVLCGLAAIGEDMDQPYIRKAVNWIKSRQNIDGGWGETCQSYHDRTLAGVGESTPSQTGWALLGLLAAGEMHSATVVRGVQYLISTQNSDGTWDEQQYTGTGFPKYFMIKYHIYRNCFPLMALGTYRTLTRTQP

SEQ ID NO: 182 - protein, 688 aa, Geotalea daltonii
  source 1..688: note = strain: frc-32; note = ACM22003.1 squalene cyclase [Geotalea daltonii FRC-32]
MSPCKHPISHALTSFNGETADSVPVQTPKTGAKIHHLPPSIWKKKEGELKSPLDIAIENSRDFFFREQLPDGYWWAELESNCTITAEYVMLYHFMDLVDRERERKMANYLLSKQTEEGFWTIYYGGPGDLSTTVEAYFALKLAGYPADHPAMVKARAFILDNGGIIKTRVFTKIFLALFGEFAWFGVPSMPIELILLPNWAYFNMYELSSWSRATIIPLSIVMTQRPVRKLPPASRVQELYVRPPSPIDYTFTKEDGIFTWKNFFIGVDHILKVYESNPIRPFKKKAMLAAENWVLEHQEATGDWGGIQPAMLNSVLALHCLGYANNHPAVAKGLEALENFCIESEDSLVLQSCISPVWDTALALKALVDSDVPNDHPALVKAAQWLLDKEIRKAGDWKVKSPELEPGGWAFEFLNDWYPDVDDSGFVMMALKDVAVKDRKSMDTAIKRGISWCLGMQSKNGGWGAFDKDNTKYLLNKIPFADLEALIDPPTVDLTGRMMELMGTFGYAKDYPPAVRALDFIKRNQEPDGSWWGRWGVNYIYGTWSVLCGLSAMGEDLNQPYIRKAINWLKSRQNIDGGWGETCESYHDSSLAGIGASTASQTGWALLALMAVGEENASAVARGVQYLLATQKSDGTWDEDLYTGTGFPKFFMIKYHIYRNCFPLTALGTYRRKTGGRAEMQVSEHNK

SEQ ID NO: 183 - protein, 680 aa, Geobacter sp.
  source 1..680: note = strain: m18; note = EET34621.1 squalene-hopene cyclase [Geobacter sp. M18]
MTSPFKHPISHALTSFNGIVTEPEQSVEQKAGAKVHQFPASLWKSKPGKAKSPLDIAIEGCRDFFFREQLPKGYWWAELESNVTITAEYIMLFNFLSLVDHERQRKMSNYLLSKQTEEGFWTIYYGGPGDLSTTVEAYFALKLTGYPADHPAMVKARAFILEKGGVIKSRVFTKIFLALFGEFDWLGVPSMPVELNLLPNWAYVNVYEFSSWARATIIPLSIVMLKRPVHKLPPSQRVQELFVRPPRAIDYTFTKEDGIFTWKNFFIGLDHMLKVYERSPVRPFKKRAMGKAEEWVLEHQEETGDWGGIQPAMLNAVLALSALGYDNGHPAVAHGLKALENFCIESDEQIVLQSCISPVWDTALALKALVDAGVPSDHPSLVKGAQWLLEREVRRPGDWRVKSPDLEPGGWAFEFLNDWYPDVDDSGFVMIALKGVEVKDRKAMNAAVKRGIDWCLGMQSKNGGWGAFDKDNTRHILNKIPFADLEALIDPPTADLTGRMLELMGTFGYAKTYPAAQRALKFLKENQEPEGPWWGRWGVNYLYGTWSVLCGLAAIGEDLEQPYIKKAVNWIKSRQNMDGGWGETCESYHDPTLAGMGESTASQTGWALLGLMAAGEVHSATVVRGVQYLISTQSQDGTWDETQYTGTGFPKYFMIKYHIYRNCFPLMALGTYRTLTGGTA

SEQ ID NO: 184 - protein, 680 aa, Geobacter sp.; identical to SEQ ID NO: 181
  source 1..680: note = strain: m21; note = ACT16952.1 squalene-hopene cyclase [Geobacter sp. M21]

SEQ ID NO: 185 - protein, 730 aa, Geobacter sulfurreducens
  source 1..730: note = strain: pca; note = AAR36453.1 squalene cyclase [Geobacter sulfurreducens PCA]
MAKGILNKFAVIAGTKKAGPPAGEERTVIAPIKEISGKAVHCSQAVKKAEEYLLALQNPEGYWVFELEADVTIPSEYIMLQRFLGREISPELGKRLENYLLDRQLPDGGWPLYAEDGFANISATVKAYLALKVLGHSPQAPHMIRARLMVLSLGGAARCNVFTRILLALFGQIPWHTPPAMPVEIVLLPQWFFFHLSKVSYWSRTVIVPLLLLYAKQPVCRLRPEEGIPELFSTPPDKLRHLDGFQPGYWRKNAFIIFDRLLKRFNRFIPSALHRKAIAEAEQWTRSHMQGSGGIGAIFPAMAYAVMALRVLGCGEGDPDYIRGLQAIDDLLQHRTPQEADPPRTDGTCIDSGMSAAFALTPSAHAAADGTGSSSICQPCNSPIWDTCLSLSALMEAGMPASHPAATQAVEWLLSQQILSPGDWSLKVPDLEGGGWAFQFENTLYPDLDDTSKVIMSLLRAGALENERYRDRIARGVNWVLGMQSSDGGWAAFDIDNNYHYLNDIPFADHGALLDPSTSDLTGRCIELLSMVGFDRTFPPIARGIGFLRSEQEENGAWFGRWGVNYIYGTWSVLSGLRQAGEDMQQPYIRKAVGWLASCQNHDGGWGETCYSYDDPSLAGKGASTPSQTAWSLLGLMAAGEVNSLAVRRGVRYLLDHQNQWGTWEEKHFTGTGFPRVFYLRYHGYRHFFPLWALGVYSRLSSGQKACQDERRHASPGDLHLPWLERIKKR

SEQ ID NO: 186 - protein, 679 aa, Geobacter sulfurreducens
  source 1..679: note = strain: pca; note = AAR34018.1 squalene cyclase [Geobacter sulfurreducens PCA]
MKISKNPISHALTSFNDAARETADNSAARKSGKIHHLPATIWKKKESTVSSPLDIAIERTQEFFFREQLPAGYWWAELESNATITAEYIMLFHFMGLVNREKERKMANYLLRQQTTEGYWTIWHGGPGDLSTTIEAYFALKLAGYPADHPSMSKARAFILEHGGILKARVFTKIFLALFGEFSWLGVPSMPIEMMLLPAGFTFNMYEFSSWSRATIIPLSIVMAERPVRKLPPWARVQELYVRPPRPTDYTFTKEDGILTWKNIFIGIDHVLKVYEASPIRPGRKKAMAIAEKWVLEHQEPTGDWGGIQPAMLNSVLALHVLGYANDHPAVAKGLQALANFCIEGEDELVLQSCVSPVWDTALGLMAMVDSGVPTDHPSLSKAAQWLLDREVRRPGDWKIKCPDLEPGGWAFEFMNDWYPDVDDSGIVMMAIKNVKVKDQRAKEDTITRGIAWCLGMQSKNGGWGAFDKDNTKHILNKIPFADLEALIDPPTADLTGRMLELMGTYGYPKDHPAAVRALKFIRETQEPDGPWWGRWGVNYIYGTWSVMSGLAAFGEDMSQPWIRKAVDWLVEHQNEDGGWGECCESYADPRLAGVGPSTASQTGWALLTLLAAGEVASSSVVRGVQYLLDTQKPDGTWDEDAFTGTGFPKFFMIKYHIYRNCFPLMALGRYRTLAGKGL

SEQ ID NO: 187 - protein, 679 aa, Geotalea uraniireducens
  source 1..679: note = strain: rf4; note = ABQ25226.1 squalene-hopene cyclase [Geotalea uraniireducens Rf4]
MNSCKHPISHALTSFNGETADAAKKQPVKPGAKIHHLPASIWKKKEGESKSPLDIAIENSRDFFFREQLPDGYWWAELESNCTITAEYLMLYHFMGIVDQERERKMATYLLSKQTAEGFWTIYFGGPGDLSTTVEAYFALKLAGYPADHPAMAKARAFILDNGGIIKCRVFTKIFLALFGEFAWFGVPSMPIELILLPNWAYFNMYELSSWSRATIIPLSIVMTERPVRKLPPSSRVQELYVRPPRPIDYTFSKEDGIITWKNFFIGVDHILKVYESNPIRPFKKRALATAENWVLDHQESTGDWGGIQPAMLNSVLALHCLGYANDHPAVAKGLEALANFCIETEDSLVLQSCISPIWDTALALKALVDSDVPTDHPALVKAAQWLLDKEVRKPGDWKIKCPELESGGWAFEFLNDWYPDVDDSGFVMMALKDVAVKDRKSMDGAIKRGINWCLGMQSKNGGWGAFDKDNTKYLLNKIPFADLEALIDPPTADLTGRMLELMGTFGYSKDYPAAVRALEFIKKNQEPEGSWWGRWGVNYIYGTWSVLGGLAAIGEDLNQPYIRKAVNWLKSRQNMDGGWGETCESYHDTSLAGIGESTPSQTGWALLSLMSAGEANSSTVARGIQYLIANQKSDGTWDEEQYTGTGFPKFFMIKYHIYRNCFPLTALGTYRKLTGGMA

SEQ ID NO: 188 - protein, 637 aa, Gloeobacter violaceus
  source 1..637: note = strain: pcc 7421; note = BAC91998.1 squalene-hopene cyclase [Gloeobacter violaceus PCC 7421]
MQVQPRIEKKHLDSAIEASQAYLLARQYSPGYWWAELESNVSMTAEVVLLHKIWRTDTGRPLAKATAHLLAEQRAHGGWELFYGDGGDLNTSIEAYMALKLLGLTADHPALARARAFILAKGGISRARIFTKIHLALIGCYDWRGVPSIPPWVMLLPEAFPVNIYEMSSWARGSTVPLLIVFDRKPVFAVEPAITLDELFVEGRAQARFDLPRSSSDWWANLFVDLDWGFKLAESLGAVPLREEGLKAAERWVLERQEATGDWGGIIPAMLNSLLALRCLDYDPHDPVVERGMAAVDRFAIETESTYRLQPCVSPVWDTALTMRALVDSGLPPDHPALAAAGTWLLKKQILDYGDWAVKNRTGPPGGWAFEFDNRFYPDVDDTAVVVMALDAVRLADETAKGQAIARAVCWVASMQCRGGGWAAFDIDNDAHWLNSLPYADLKAMIDPNTADVTARVLEMYGRCRLIPAAAGAQRALDYLRRTQEPEGCWFGRWGVNYLYGTSGVLSALAAFAPAERTAIERAAAWLRGCQNTDGGWGETCGSYVDRTLMGQGPSTASQTAWALLGLIDASRVARFSDSSALERGLAYLVETQKADGSWDEPYFTGTGFPGHFYLKYHLYQQHFPLSALGRYRRLLS

SEQ ID NO: 189 - protein, 663 aa, Gluconacetobacter diazotrophicus
  source 1..663: note = strain: pa1 5; note = ACI51585.1 squalene-hopene cyclase [Gluconacetobacter diazotrophicus PA1 5]
MMANATDTIELPPSRAADRIVPMTDIDQAVDAAHAALGRRQQDDGHWVFELEADATIPAEYVLLEHYLDRIDPALEERIGVYLRRIQGDHGGWPLYHGGKFDVSATVKAYFALKAIGDDIDAPHMARARAAILDHGGAERSNVFTRFQLALFGEVPWHATPVMPVELMLLPRKALFSVWNMSYWSRTVIAPLLVLAALRPRAINPRDVHVPELFVTPPDQVRDWIRGPYRSQLGRLFKYVDIALRPAERLIPDATRQRAIKAAVDFIEPRLNGEDGLGAIYPAMANTVMMYRALGVPDSDPRAATAWEAVRRLLVELDGEAYCQPCVSPIWDTGLAGHAMIEAASGPEGIRPEDTKKKLAAAAEWLRERQILNVKGDWAINCPDVPPGGWAFQYNNDYYPDVDDTAVVGMLLHREGDPANDEALERARQWIIGMQSSNGGWGAFDIDNNLDFLNHIPFADHGALLDPPTADVTARCISFLAQLGHPEDRPVIERGIAYLRTDQEREGCWFGRWGTNYIYGTWSVLCAYNAAGVAHDDPSVVRAVDWLRSVQREDGGWGEDCASYEGATPGIYTESLPSQTAWAVLGLMAVGLRDDPAVMRGMAYLTRTQKDDGEWDEEPYNAVGFPKVFYLRYHGYRQFFPLLALSRYRNLASSNSRHVAFGF

SEQ ID NO: 190 - protein, 663 aa, Gluconacetobacter diazotrophicus
  source 1..663: note = strain: pa1 5; note = CAP55563.1 putative Squalene--hopene cyclase [Gluconacetobacter diazotrophicus PA1 5]
MMANATDTIELPPSRAADRIVPMTDIDQAVDAAHAALGRRQQDDGHWVFELEADATIPAEYVLLEHYLDRIDPALEERIGVYLRRIQGDHGGWPLYHGGKFDVSATVKAYFALKAIGDDIDAPHMARARAAILDHGGAERSNVFTRFQLALFGEVPWHATPVMPVELMLLPRKALFSVWNMSYWSRTVIAPLLVLAALRPRAINPRDVHVPELFVTPPDQVRDWIRGPYRSQLGRLFKYVDIALRPAERLIPDATRQRAIKAAVDFIEPRLNGEDGLGAIYPAMANTVMMYRALGVPDSDPRAATAWEAVRRLLVELDGEAYCQPCVSPIWDTGLAGHAMIEAASGPKGIRPEDTKKKLAAAAEWLRERQILNGEGRLGDQLPRRAPRRLGLPVQQRLLPRRGRHGSGRHVLHREGDPANDEALERARQWIIGMQSSNGGWGAFDIDNNLDFLNHIPFADHGALLDPPTADVTARCISFLAQLGHPEDRPVIERGIAYLRTDQEREGCWFGRWGTNYIYGTWSVLCAYNAAGVAHDDPSVVRAVDWLRSVQREDGGWGEDCASYEGATPGIYTESLPSQTAWAVLGLMAVGLRDDPAVMRGMAYLTRTQKDDGEWDEEPYNAVGFPKVFYLRYHGYRQFFPLLALSRYRNLASSNSRHVAFGF

SEQ ID NO: 191 - protein, 664 aa, Gluconobacter oxydans
  source 1..664: note = strain: 621h; note = AAW61994.1 Squalene-hopene cyclase [Gluconobacter oxydans 621H]
MLIYSDILEKEDRVSETLSRQSVEPDEINHAIEGAQAALGGKQKSDGHWVYELEADATIPAEYVLLEHYLDRIDPEKQAKIGVYLRRIQGHHGGWPLYHDGGFDLSATVKAYFALKAIGDDINAPHMRIAREAILDHGGAARTNVFTRIQLALFGEVPWDATPVMPVELMLLPRKAFFSVWNMSYWSRAVIAPLLVLNALRPKAINPRGIHVQELFVKPPSEVKDWIRGPYRSVWGRFFKHLDSALRPVLPLIPRSVHKKALKAASDFIEPRLSRGGLGAIYPAMANVVMMYRAQGVPDSDPRAKTAWDAIQDLLVDHGDEIYCQPCVSPVWDTGLSGLAMIEAASGPAGTKTKETLAALKKSAEWLREHQILDVKGDWAINAPDLRPGGWAFQYENDYYPDVDDTAVVAMLLHRVDPENSREAISRAREWIIGMQSTNGGWGAFDIDNDHELLNHIPFSDHGALLDPPTADVSARCISFLAQLGDPDDRPVILKAIEYLRSEQEPEGCWFGRWGTNYIYGTWSVLCALNIAGVPHDDPMVLRAVNWLESVQRPDGGWGEDCATYEGGTAGTYKKSLPSQTAWAVLALMAVGRRESEAVKRGVAYLVSQQNEKGEWQEEAYNAVGFPKVFYLRYHGYKQFFPLTALARYRNLGVSNSGKVEYGF

SEQ ID NO: 192 - protein, 651 aa, Granulibacter bethesdensis
  source 1..651: note = strain: cgdnih1; note = ABI63005.1 Squalene--hopene cyclase [Granulibacter bethesdensis CGDNIH1]
MPKDIPADLASEAISGDMLEQAVLRASMALHRKQQTDGHWVFELEADATIPAEYVLLEHFLDRIDDDLERKIGVYLRRIQGDHGGWPLFHEGAFNLSASVKAYYALKAIGDDPDAPHMRRAREAILAAGGAERSNVFTRIQLALFGQIPWRGVPVMPAELMIAPKWFPINMWKVSYWSRTVIAPLLVLMDRKPKARNPRNVHVRELFLHDPDRIRDWIRGPFRSGWGHFFKYLDSVLRVVEPVALKPMRPRSIRLAVDFVRERLNGEDGLGAIYPAMANSVMMYDVLGYSPDHPEAAIAWESVRKLLVIKEDEAYCQPCLSPIWDTGLSGHAMAEAEGAVSPGVAAACDWLRNRQITDVVGDWAEIRPGVQPGGWAFQYNNAHYPDVDDTAVVAMLLHRQGDPAHEESIRKAREWIIGLQCRDGGWGAFDADNDKDYLNHIPFADHGALLDPPTADVTARCISFLAQLGNPEDKPVIDRAMAWLRKEQEADGSWFGRWGTNYIYGTWSVLCAMNVAGMPHDDPAIRRAVNFLVATQREDGGWGEDEETYDPASGAQPGRYKESTPSQTAWALIGLMAAGEAEHEATRRGIAYLQATQKPDGEWDEAAYTAVGFPRVFYLKYHGYRQFFPLMALSRYKNLRSSNMKKVSFGF

SEQ ID NO: 193 - protein, 665 aa, Hyphomicrobium denitrificans
  source 1..665: note = strain: atcc 51888; note = EET65847.1 squalene-hopene cyclase [Hyphomicrobium denitrificans ATCC 51888]
MTTNAAATSARSGEDAIRQVSGQQLETAIASARNSLLALQRPDGHFVFELEADATIPAEYVLMRHYLAEPVDAVLEEKIARYLRRIQSDDGGWPLFRDGASNISASVKAYYALKMIGDAPNAPHMQKARAWILAQGGASHSNVFTRNLLALFGAIPWSGVPVMPVEIMLLPKWFPFHIDKISYWARTVLIPLTVLNALKPVARNPKGVGIAELFVTPPDQVRNWPKGPHQKFPWSQVFGGIDRVLRLFEPAFPKSLRKKSIDKAVAFATERLNGEDGLGGIFPAMVNALLVYDALGYPHDHPDYVTARGSIEKLLVIKDDEAYCQPCLSPVWDTALAVHALMESGVAQADQNVDRALAWLKPLQVLDTVGDWAASRPGVRPGGWAFQYANAYYPDVDDTAVVVMAMDRAAGGDAAKRDHYRESMARGREWVAGVQSKNGGWGAFDADNTYEYLNQIPFSDHGALLDPPTADVSARCVSMLAQLGERRETSPVLDKAMRYLESTQEKDGSWYGRWGMNYIYGTWSVLCALNAAGVAPSAPSMRKAADWLLSIQNSDGGWGEDGESYSLDYKGYEPAPSTASQTAWALMGLMAAGEVDHPAVQRGVAYLAAKQGSDGFWGEERFTATGFPRVFYLRYHGYSKFFPLWALARYRNLNAANSKSVLVGM

SEQ ID NO: 194 - protein, 694 aa, Leptospirillum ferrodiazotrophum
  source 1..694: note = EES53667.1 squalene-hopene cyclase [Leptospirillum ferrodiazotrophum]
MSGQSNFTGGKKMTPAEGSSSPAPALLEKAAPSIELDERSDPLSRTLARAVSWLVAAQDGAGHWVAPLEADATIPSEYVFLHEVLGRPLDPVRRDKIVRAILSVQGKEGAWPLFHDGDPDISATVKAYQALKLCGFDPSHPALVRAREWVLSQGGAGKVNVFTRIALAIFGQYSWTKIPALPAEMVLLPSWFPFSIYSVSYWSRTVIVPLLFIYHHKPLVRLSPERGISELFDPARPDGESFAPSPDFFSLRNLFLLLDKVLQVWNRHPPGFLRKKALSFAMEWMVPRLKGEGGLGAIYPAMANSAVALSLEGYELDHPLMQRVLASIDDLLIEGEKEVLVQPCVSPVWDTALAMGALIEAGISPDSPTVDRAMEWFCAREVRTRGDWAIRAPDCEPGGWAFQFENDYYPDVDDTAMVLMGMAKILPARPDLAARMEGVFRRATLWVMAMQGTDGGWGAFDRDNDLLFLNHIPFADHGALLDPSTADLTGRVLELLGALGYGPDFPPAARAIRYLRREQEEDGSWFGRWGVNYIYGTWSVVAGLKSIGVPMSEPWVMRSMEFLLARQNPDGGWGEDCLSYASRDFAGRGASTPSQTAWALIALLHGGHAGHMAVRQGVDYLIQQMTPEGTWNEELFTGTGFPRVFYLRYHMYRHYFPLWALALYRNMTERGRALGHERVDFWKTAPYAPIARSV

SEQ ID NO: 195 - protein, 683 aa, Leptospirillum rubarum
  source 1..683: note = EAY57382.1 Squalene-hopene cyclase [Leptospirillum rubarum]
MKGKEPTREELLSFSSGIQMDSSAENTTPVSTEELQEKVRLAAESLISRQVEEGYWVEPLEADVTITSEYILLQYLLGRERDEFFRRAAPFILESQGEDGGWPLYHGGPAEISATVKAYLALKLLGYDADHPAMQRARALVLERGGAINVNVFTRITLALFGQYDWKGVPALPPEMILLPRWFPLSIYTVSYWSRTVIVPLLFIYHYKPLLELPPEKGVQELFITPMSEVRVHYAWDKHWVSWKNLFFVLDRILQAWNRHPPSFLRRKALKKAMEWMIPRLKGEGGLGAIYPAMANSVLALRLEGYAMDHPLVRRAIQSIDDLVFDLGEQQSVQPCHSPIWDTALALGALYEAGLDEGSPFVSRALDWFCRKEVRTVGDWSVRVPGVEAGGWAFQFENDYYPDIDDTSVVLMDFAKWVPEMGAYRDVFRRAIEWTLSMQGTDGGWGAFDKDNDFLFLNNIPFADHGALLDPSTSDVTGRVTELLGILGYDARTPVVRRALRFLRKEQEENGSWYGRWGVNYIYGTWSVVSALKAVGEDMSAPYVQKAMQFLFSRQNPDGGWGESCYSYFRKDTAGEGVSTSSQTAWALIALIHGGHVRHPAVSKGIDFLLSRQQADGKWLEQEYTGTGFPKVFYLRYNMYRDYFSLWALSLYRNVLLDGQSRVERLARRWKGNPYPVRSRFLA

SEQ ID NO: 196 - protein, 683 aa, Leptospirillum sp.
  source 1..683: note = strain: group ii '5-way cg'; note = EDZ38599.1 Squalene-hopene cyclase [Leptospirillum sp. Group II '5-way CG']
MEGKDPTREELLSFTSGIQMDSRVGNTNPVSTEELQEKVRLAAESLISRQGEEGYWVEPLEADITITSEYVLLQYLLGRERDEFFRRAAPFILESQGEDGGWPLYNGGPAEISATVKAYLALKLLGYDADHPAMQRARALVLERGGAINVNVFTRITLALFGQYDWKGVPALPPEMILLPRWFPLSIYTVSYWSRTVIVPLLFIYHYKPLLELPPEKGVQELFITPMSEVRVHYAWDKHWVSWKNLFFVLDRILQAWNRHPPSFLRRKALKKAMEWMIPRLKGEGGLGAIYPAMANSVLALRLEGYEMDHPLVRRAIQSIDDLVFDLGEQQSVQPCHSPIWDTALALGALYEAGLDEGSPFVSRALDWFCRKEVRTVGDWSVRVPGVEAGGWAFQFENDYYPDIDDTSVVLMDFAKWVPEMGAYRDVFRRAIEWTLSMQGTDGGWGAFDKDNDFLFLNNIPFADHGALLDPSTSDVTGRVTELLGILGYDARTPVVRRALRFLRKEQEENGSWYGRWGVNYIYGTWSVVSALKAVGEDMSAPYVQRAMQFLFSRQNPDGGWGESCYSYFRKDTAGEGVSTASQTAWALIALIHGGHVRHPAVSKGIDFLLSRQQADGKWLEQEYTGTGFPKVFYLRYNMYRDYFSLWALSLYRNVLLDGQSRVERLSRRWKGTPYPVRSRFLA

SEQ ID NO: 197 - protein, 667 aa, Magnetospirillum magnetotacticum
  source 1..667: note = strain: ms-1; note = ZP_00052068.2 COG1657: Squalene cyclase [Magnetospirillum magnetotacticum MS-1]
MREAAVSKVETLQRPKTRDVSLDDVERGVQSAARALTDMTQADGHICFELEADATIPSEYILFHHFRGTEPRAGLEAKIGNYLRRTQSKVHGGWALVHDGPFDMSASVKAYFALKMIGDDIEAPHMRAVRKAILQRGGAANANVFTRILLALYGEVPWTAVPVMPVEVMHLPKWFPFHLDKVSYWARCTMVPLFVIQAKKPRAKNPRGVGVAELFVTPPDSVRTWPGSPHATWPWTPIFGAIDRVLQKTQDHFPKVPRQRAIDKAVAWVSERLNGEDGLGAIFPSMVNSVLMYEVLGYPPDHPQVKIALEAIEKLVAEKDDEAYVQPCLSPVWDTALTSHAMLEVGGTQAEANARAGLDWLKPLQILDIKGDWAETKPNVRPGGWAFQYANPHYPDLDDTAVVVMAMDRAQRQHGLVSGMPDYSTSIARAREWVEGLQSADGGWAAFDADNNHHYLNHIPFSDHGALLDPPTADVTARVVSMLAQLGETRETSRALDRGVTYLLNDQEKDGSWYGRWGMNFIYGTWSVLCALNAAGVDPQSPEIRKAVAWLIRIQNPDGGWGEDASSYKLNPEFEPGYSTASQTAWALLALMAVGEVDDPAVARGVNYLMRTQGADGLWNEERYTATGFPRVFYLRYHGYPKFFPLWAMARFRNLKRGNSRQVQFGM

SEQ ID NO: 198 - protein, 691 aa, Methylacidiphilum infernorum
  source 1..691: note = strain: v4; note = ACD82457.1 Squalene-hopene cyclase [Methylacidiphilum infernorum V4]
MHSGRVFLEKENREENRATFHSSPLILVEESLNLPKKVEETIKKAQRYLLSIQKEDGHWVGELFVDVTLACDCIHLMHWRGKIDYKKQLRLVKHIVDRQLPDGGWNIYPGGPSEVNATVKAYFALKLAGFSPDDPLMAKARSTILRLGGIPKCMTYTKLGLALLGVYPWDRLPVIPPEIILFPNWFPFNIYEISAWSRAMLVPLSVIHHFKPTRNLPEKYQLHELFPYGTEHGKFSWLKKGARYLSKQGLFLACDKFLQYWDKTSLKPFRKMALKKAEKWLLERISAGSDGLGAIFPAMHYAIMALIAMGYTEDNPILKKAIADFEGLEVDDKKNDDLRIQPCLSPVWDTAVGLVALAESGVARNAKELKRAAYWLLDREIKIKGDWHVRNPHPEPSGWAFEYNNVYYPDVDDTLMVLLALRLIDIEDKIRKEEVMQRALRWVISFQCKNGGWAAFDKDVYKKWLEDIPFADHNAILDPPCSDITARALELFGKMGIKKTERFVQKAIAYLKETQENDGSWMGRWGVNYIYGTWQALRGLQAIGENMNQEWILRARDWLESCQNEDGGWGETPASYDNPQLKGKGPSTASQTAWAVSGIMACGDIFRPSVSRGIKYLCDRQLSDGSWAEEFLTGTGFPGVFYLKYDMYRNAWPLLVIGEYHRQYLKAKEQVSYWVDGTIGRKVKKERLPEI

SEQ ID NO: 199 - protein, 667 aa, Methylorubrum extorquens
  source 1..667: note = strain: cm4; note = ACK83067.1 squalene-hopene cyclase [Methylorubrum extorquens CM4]
MREAAVSKVETLQRPKTRDVSLDDVERGVQSATRALTEMTQADGHICFELEADATIPSEYILFHQFRGTEPRPGLEAKIGNYLRRTQSKVHGGWALVHDGPFDMSASVKAYFALKMIGDDIEAPHMRAVRKAILQRGGAANANVFTRILLALYGEVPWAAVPVMPVEVMHLPKWFPFHLDKVSYWARCTMVPLFVIQAKKPRAKNPRGVGVAELFVTPPDSVRTWPGSPHATWPWTPIFGGIDRVLQKTQDHFPKVPRQRAIDKAVAWVSERLNGEDGLGAIFPAMVNSVLMYEVLGYPPEHPQVKIALEAIEKLVAEKEDEAYVQPCLSPVWDTALNSHAMLEAGGHQAEANARAGLDWLKPLQILDIKGDWAETKPNVRPGGWAFQYANPHYPDLDDTAVVVMAMDRAQRQHGLVSGMPDYSESIARAREWVEGLQSADGGWAAFDADNNHHYLNHIPFSDHGALLDPPTADVTARVVSMLSQLGETRATSRALDRGVTYLLNDQEKDGSWYGRWGMNFIYGTWSVLCALNTAGVDPQSPEIRKAVAWLIRIQNPDGGWGEDASSYKLNPEFEPGYSTASQTAWALLALMAAGEVDDPAVARGVNYLVRTQGQDGLWSEERYTATGFPRVFYLRYHGYPKFFPLWAMARFRNLKRGNSRQVQFGM

SEQ ID NO: 200 - protein, 645 aa, Methylorubrum extorquens
  source 1..645: note = strain: cm4; note = ACK86232.1 squalene-hopene cyclase (plasmid) [Methylorubrum extorquens CM4]
MNTEPRFSAPETLRAIAGAGRALGRHQRRDGHWVFELEADATIPAEYVLLEHYMDRITPERQARIGAYLRRIQGEHGGWPMFHAGEFNISASVKAYCALKAIGDDPQAPHMVRARQAILGHGGAERANVFTRIQLALFGAIPWRGVPVMPVEIMHLPKWFFFNIWAMSYWARTCVVPLLVLQARKPRARNPRQVSFDEIFRTEPDEVRDWIRGPYRSRWGVVFKHIDTVLRWTEPLFSKVARESAIFKAVDFVEERLNGEDGLGAIYPAMAYALMMYDVLGYPEDDPRCVTIWKAIDKLLIETDEEVYCQPCVSPVWDTSLSGHAMIEAARTGGIEAQAELDAACDWLVARQVKDVRGDWAETRPDAEPGGWAFQYRNDHYPDVDDTAVVAMLLHRNGRPEHAEAIEKARRWVVGVQSRNGGWGAFDADNDREFLNHIPFSDHGALLDPPTADVTGRCISFLSQLGHEEDRPVIERALAYLRAEQERDGSWYGRWGTNYVYGTWTVLCGLNAAGIPHDDPMVRRAVDWLVSIQRADGGWGEDERSYDVGHYVENAESLPSQTAWAMLGLMSVGQADHPAVLRGAAYLQRTQGPDGEWQERAYNAVGFPRVFYLKYHGYRLFFPLFALSRLHNLQRGNSREVSFGF

SEQ ID NO: 201 - protein, 667 aa, Methylorubrum extorquens
  source 1..667: note = strain: am1; note = CAX24364.1 squalene-hopene-cyclase [Methylorubrum extorquens AM1]
MREAAVSKVETLQRPKTRDVSLDDVERGVQSATRALTEMTQADGHICFELEADATIPSEYILFHQFRGTEPRPGLEAKIGNYLRRTQSKVHGGWALVHDGPFDMSASVKAYFALKMIGDDIEAPHMRAVRKAILQRGGAANANVFTRILLALYGEVPWVAVPVMPVEVMHLPKWFPFHLDKVSYWARCTMVPLFVIQAKKPRAKNPRGVGVAELFVTPPDSVRTWPGSPHATWPWTPIFGGIDRVLQKTQDHFPKVPRQRAIDKAVAWVSERLNGEDGLGAIFPAMVNSVLMYEVLGYPPEHPQVKIALEAIEKLVAEKEDEAYVQPCLSPVWDTALNSHAMLEAGGHQAEANARAGLDWLKPLQILDIKGDWAETKPNVRPGGWAFQYANPHYPDLDDTAVVVMAMDRAQRQHGLVSGMPDYSESIARAREWVEGLQSADGGWAAFDADNNHHYLNHIPFSDHGALLDPPTADVTARVVSMLSQLGETRATSRALDRGVTYLLNDQEKDGSWYGRWGMNFIYGTWSVLCALNAAGVDPQSPEIRKAVAWLIRIQNPDGGWGEDASSYKLNPEFEPGYSTASQTAWALLALMAAGEVDDPAVARGVNYLVRTQGQDGLWSEERYTATGFPRVFYLRYHGYPKFFPLWAMARFRNLKRGNSRQVQFGM

SEQ ID NO: 202 - protein, 663 aa, Methylobacterium nodulans
  source 1..663: note = strain: ors 2060; note = ACL61886.1 squalene-hopene cyclase [Methylobacterium nodulans ORS 2060]
MGKVETLHRMSTQDITLDDVERRVSLASKALMRLAGPDGHWCFELEADATIPSEYILYHHFRGSIPSAELEGKIANYLRRTQSAQHDGWSLVHDGPFDMSATVKAYFALKMIGDSIEAPHMRRAREAILRRGGAAHANVFTRTLLALYGEVPWSAVPVMPVEVMLLPRWFPFHLDKVSYWARTVMVPLFVLQAKKPRARNPRGIGIQELFVEPPERVKRWPAGPQESSPWRPVFAAIDKVLQKVEGSFPAGSRARAIDKAVAFVSERLNGEDGLGAIFPAMVNAVLMYEALGYPEDHPLVATARSSVEKLVTVKEHEAYVQPCLSPVWDTALSAHALMEAGGVEAERHAKRALDWLKPLQVLDIKGDWAASKPNVRPGGWAFQYANPHYPDLDDTAVVVMAMDRAQVRRSPGPDAADYGQSIARAREWVEGLQSRDGGWAAFDADNTYHYLNYIPFSDHGALLDPPTADVTARCVSMLAQLGETRESCPPLDRGVAYLLADQEADGSWYGRWGMNYIYGTWSVLCALNAAGVDPASEPVRRAVNWLTTIQNPDGGWGEDAASYKLEYRGYERAPSTASQTAWALLGLMAAGEADSPAVARGINYLTRSQGADGLWTEDRYTATGFPRVFYLRYHGYAKFFPLWALARYRNLQQSNSRRVAVGM

SEQ ID NO: 203 - protein, 667 aa, Methylorubrum populi
  source 1..667: note = strain: bj001; note = ACB79998.1 squalene-hopene cyclase [Methylorubrum populi BJ001]
MREAAVSKVETLQRPKTRDVSLDDVERGVQNAARALTEMTQTDGHICFELEADATIPSEYILFHQFRGTVPRDGLEAKIGNYLRRTQSKVHGGWALVHDGPFDMSATVKAYFALKMIGDDIEAPHMRAARKAILQRGGAANANVFTRILLALYGEVPWAAVPVMPVEVMHLPKWFPFHLDKVSYWARCTMVPLFVIQAKKPRAKNPRGIGVAELFVTPPDSVRTWPGSPHATWPWTPIFGAIDRVLQKTQDHFPKVPRQRAIDKAVAWVSERLNGEDGLGAIFPSMVNSVLMYEVLGYPPDHPQVKIALEAIEKLVAEKDDEAYVQPCLSPVWDTALTSHAMLETGGAAAEANARAGLDWLKPLQILDIKGDWAETKPNVRPGGWAFQYANPHYPDLDDTAVVVMAMDRAQRQHGLVSGMPDYSASIARAREWVEGLQSADGGWAAFDADNNHHYLNHIPFSDHGALLDPPTADVTARVVSMLSQLGETRETSRALDRGVTYLLNDQEKDGSWYGRWGMNFIYGTWSVLCALNAAGVDPQSPEIRKAVAWLIRIQNPDGGWGEDASSYKLNPEFEPGYSTASQTAWALLALMAVGEVDDPAVARGVNYLMRTQGQDGLWNEERYTATGFPRVFYLRYHGYPKFFPLWAMARFRNLKKGNSRQVQFGM

SEQ ID NO: 204 - protein, 665 aa, Methylobacterium radiotolerans
  source 1..665: note = strain: jcm 2831; note = ACB27373.1 squalene-hopene cyclase [Methylobacterium radiotolerans JCM 2831]
MREAVSKVEALQRSKTQGISLEDVERGVAQATRALTALAHDDGHICFELEADATIPSEYILFHHFRGTQVPGDLEAKIGNYLRRTQGRHGGWALVHEGPFDMSCTVKAYFALKMIGDDIEAPHMRRAREGILSRGGAANANVFTRFMLALYGEVPWRAVPVMPVEVMFLPKWFPFHLDKISYWARTTVVPLFVLQATKPRARNPRGISVQELFVTPPESVRSWPGSPHATWPWTPIFGFIDRVLQRVENHLPRKSRQRAMEMARAWVSERLNGEDGLGAIFPAMVNSVLMYEVMGYRPDHPQVRVACDAIEKLVVEKADEAYVQPCVSPVWDTALASHALLEAGGPEAEAQARAGLDWLKPRQVLDIVGDWAARKPKVRPGGWAFQYANAHYPDLDDTAVVVMAMDRAMHQHGLVAGMPDYKASIARAREWVEGLQSEDGGWAAFDADNNHMYLNHIPFSDHGALLDPPTADVTARVVGMLSQLGETRETSRALDRGVNYLLNDQEEDGSWYGRWGMNFIYGTWSVLCALNAAGVDPADPRIQKAVSWLIRIQNPDGGWGEDASSYKIDPAFEPGSSTASQTAWALLALMAAGAVDDPAVTRGINFLTRTQGADGFWKEERYTATGFPRVFYLRYHGYPKFFPLWAMARFRNLKRGNSRRVQFGM

SEQ ID NO: 205 - protein, 663 aa, Methylobacterium sp.
  source 1..663: note = strain: 4-46; note = ACA20611.1 squalene-hopene cyclase [Methylobacterium sp. 4-46]
MGKVETLHRTSTQDITLDDVERRVTLASKALMRLANADGHWCFELEADATIPSEYILYHHFRGSIPTAELEGKIAAYLRRTQSAQHDGWALIHDGPFDMSATVKAYFALKMVGDPIDAPHMRRARDAILRRGGAAHANVFTRIMLALYGEVPWTAVPVMPVEVMLLPRWFPFHLDKVSYWARTVMVPLFVLQAKKPRARNPRGIGIRELFVEAPERVKRWPAGPQESSPWRPVFAAIDKVLQKVEGFFPAGSRARAIDKAVAFVSERLNGEDGLGAIFPAMVNTVLMFEALGYPDDHPFAVTARSSVEKLVTVKEHEAYVQPCLSPVWDTALAAHALMEAGGTEAERHAKRAMDWLKPLQVLDIKGDWAASKPDVRPGGWAFQYANPHYPDLDDTAVVVMAMDRVQSRRSPGPDAADYGLSIARAREWVEGLQSRDGGWAAFDADNTYHYLNYIPFSDHGALLDPPTADVTARCVSMLSQLGETRETCPPLDRGVAYLLADQEADGSWYGRWGMNYIYGTWSVLCALNAAGIDPACEPVRRAVTWLTAIQNPDGGWGEDASSYKLEYRGYERAPSTASQTAWALLALMAAGEADNPAVARGINYLTRTQGADGLWAEDRYTATGFPRVFYLRYHGYAKFFPLWALARYRNLQRGNSLKVAVGM

SEQ ID NO: 206 - protein, 672 aa, Methylocella silvestris
  source 1..672: note = strain: bl2; note = ACK52150.1 squalene-hopene cyclase [Methylocella silvestris BL2]
MDDRVGAATFEAQPRAGFGSVEAAISRAREALLAVQKPDGHFVFELEADVSIPAEYILFRHFLGDPAKTEIERKIGVYLRRRQTAAGGWPLFAEGVFNVSSSVKAYFALKIIGDDPNAPHMAKARNAILAHGGAAQSNVFTRSLLALYGEVPWRAVPAMPVEIMHLPRWFPFHLSKVSYWGRTVIAPLIVVHALKPRAKNPRKISVSELFVAPAETVSRWPGAPHKSFPWTTIFGAIDRVLHKTEPLLPARSHQTAIDKAVAFVTARLNGEDGLGAIYPAMAYSAMMFFALGAPLSDPRIVQIRKAIDRLLVIKDGEAYCQPCVSPVWDTALASHALMESAGQRPEARTAPAAAAVFEALDWLKPLQVLDVKGDWATQNPDVRPGGWAFQYANPHYPDLDDTAVVVLAMDRAVKTSPLIAGEEETAYVEAISRAREWILGLQSANGGFGAFDADNDRDYLNYIPFADHGALLDPPTADVTARCVSMLGQLGERPETSPALARAIDYLLSEQEEEGSWFGRWGMNYIYGTWSVLSAFNAVERPADCAATRKAAAWLKRIQNPDGGWGEDGESYALGYKGYNPAPSTASQTAWALLALMAAGEVDAPEVALGLDYLVSTQADDGFWDEARFTATGFPRVFYLRYHGYAKFFPLWAMARYRNLKSGNRLKTQFGM

SEQ ID NO: 207 - protein, 654 aa, Methylococcus capsulatus
  source 1..654: note = strain: str. bath; note = CAA71098.1 squalene-hopene cyclase [Methylococcus capsulatus str. Bath]
  VARIANT 591: note = X may be any amino acid.
MLREATAISNLEPPLTASYVESPLDAAIRQAKDRLLSLQHLEGYWVFELEADCTIPAEYILMMHFMDEIDAALQAKIANYLRHHQSADGSYPLFRGGAGDISCTVKVYYALKLAGDSIDAPHMKKAREWILAQGGAARSNVFTRIMLAMFEQIPWRGIPFTPVEIMLLPKWFPFHLDKVSYWSRTVMVPLFILCSHKVTARNPSRIHVRELFTVEPQKERHYFDHVKTPLGKAILALERFGRMLEPLIPKAVRKKATQKAFDWFTARLNGVDGLGAIFPAMVNAYEALDFLGVPPDDERRRLARESIDRLLVFQGDSVYCQPCVSPIWDTALTSLTLQEVARHTADLRLDAALSKGLKWLASKQIDKDAPGDWRVNRAGLEGGGWAFQFGNDYYPDVDDSAVVAHALLGSEDPSFDDNLRRAANWIAGMQSRNGGFGAFDADNTYYYLNSIPFADHGALLDPPTADVSARCAMFLARWVNRQPELRPVLERTIDYLRREQEADGSWFGRWGTNYIYGPGAVLLAYEGRRVPNDDPSVRRAVAWLKSIQREDGGWGEDNFSYHDPSYRGRFHTSTAFQTGFALIALMAAGEXGSPEVQAGVDYLLRQQRPDGFWNDECFTAPGFPRVFYLKYHGYDKFFPLWALARYRNERYALA

SEQ ID NO: 208 - protein, 695 aa, Aspergillus fischeri
  source 1..695: note = strain: nrrl 181; note = EAW20752.1 squalene-hopene-cyclase - putative [Aspergillus fischeri NRRL 181]
MLQTEAITTEGLRFRSLAPDDPLLPRVKQALKLSGQHSREEMHSDGHWCGEVKTNATTSAEHVLLCQALDINLDADREAFISWFRCTQGADGGWSTAPDQAGDISVTVEAYLALKILGLSEDDAAMRSARDFAIAAGGVARVRIFTRIYLAMFGLFPWAAVPELPPELILLPSRVPVSIYHWSAWARATVVPLLIISHHRPIYALPGGKATCSDYLDELWCDPRNKMVPYNHDKPTAWRSDPFALIFTLADSILHRLDGLRSFNPLRRFALRKCVDWILEHQEDMGDIGDIMPPLHGAMLALRLEGYPLHSDPIHRGLEAIERFAYRDQQGKRIQTTVSAFWDTSLMLVALGDAGMASSPWLTRSLGWLQQHQRLGNYGDWKVNNPGLKAGGFSFGYFNTWYPDVDDTASAVLAIIRQDERLVCSASVLDALNWLLGMQNTDGGWGAFDRDNNKLFLNKIPFSDMEAFCDPSTPDVTGHVLEAFGIFLAVSARQQSPTKADVLTDRIVSASRRAICYLSDTHVSSGGWYGRWGCNYIYGTSAVLCALAYFGSKSDTLSGVRSVKDAVNQAIRWLETVQNQDGGWGETVNSYKDPSRAGSGPSTASQTAWAIMALLPYLPPSTEVIQRGVEYLLRTQTKTASQGATWHEKAYTGTGFPKYFYMGYSFYCHYFPMMALGRYAYPCPEWHENWRPKKE

SEQ ID NO: 209 - protein, 654 aa, Nitrobacter hamburgensis
  source 1..654: note = strain: x14; note = ABE63461.1 Squalene cyclase [Nitrobacter hamburgensis X14]
MNSVNATVAPIDDAALGGSIGAATRGLLDLKQPDGHFVFELEADATIPSEYVLLRHYLGEPVDAALEAKIAVYLRRIQGAHGGWPLVHDGPFDMSASVKAYFALKMIGDSIDAPHMARAREAILSRGGAANVNVFTRFLLSLFEVLTWRSAPVLPIEIMLLPMWSPFHINKISYWARTTMVPLMVLAALKPRARNPRGIGIRELFLQDPATVGTPKRAPHQSPAWFTLFNSLDWILRKIEPLFPKRLRARAIEKAIAFVEERLNGEDGLGAIFPPMVNTVMMYDALGFPPEHPPRAVARRGIDKLLVIGKDEAYCQPCVSPIWDTALTCHALLEAGGPEALSGAGKSLDWLLPKQELVLKGDWAVKRPDVRPGGWAFQYANAHYPDLDDTAVVVMAMDRVRRNDRSDKYNEAIARGREWIEGMQSRDGGFAAFDADNLEYYLNNIPFSDHAALLDPPTEDVTARCVSMLAQLGETVRSSPSMAAGVDYLRRTQLKEGSWYGRWGLNYIYGTWSVVCALNAAGVDHQDPAMRKAVDWLVSIQNADGGWGEDAVSYRLDYKGFEGAPTTASQTAWALLALMAAGEVENPAVARGMKYLIDTQTKKGLWDEQRFTATGFPRVFYLRYHGYSRFFPLWALARYRNLRSTNSKVVGVGM

SEQ ID NO: 210 - protein, 651 aa, Nitrobacter sp.
  source 1..651: note = strain: nb-311a; note = EAQ34404.1 terpene synthase/Squalene cyclase [Nitrobacter sp. Nb-311A]
MNATVAQIGDAVLEDRIGSATRGLLNLKQSDGHFVFELEADATIPSEYILLRHYLGEPVDTVLEAKIAAYLRRIQGAHGGWPLVHDGPFDMSASVKAYFALKMIGDSVDAPHMARAREAILSRGGAANVNVFTRFLLSFFEVLTWRSVPVLPVEIMLLPMWSPFHLNKISYWARTTMVPLMVLAVLKPRARNPRDVGIRELFLQDPATVRTPKRAPHQSPAWFALFSSLDWILRRIEPLFPKRLRARAMEKAIAFVEERLNGEDGLGAIFPPMVNTVMMYDALGFPPEHPPRAVTRRGIDKLLVIGEDEAYCQPCVSPIWDTALSCHALLEAGAPEALNSAGKCLDWLLPKQELVLKGDWAAKRPDVRPGGWAFQYANGHYPDLDDTAVVVMAMDRVRRNGRGDKYDEAIERGREWIEGMQSRDGGFAAFDADNLEYYLNNIPFSDHAALLDPPTEDVTARCVSMLAQLGATVDGSSSMAAGVEYLRRTQLAEGSWYGRWGLNYIYGTWSVLCALNAAGVDHQDPAIRKAVDWLLSIQNEDGGWGEDAVSYRLDYKGFEGAPTTASQTAWALLALMAAGEVENPAVTRGIKYLIDTQTKKGLWDEQRYTATGFPRVFYLRYHGYSKFFPLWALARYRNLRSTNSKVVGVGM

SEQ ID NO: 211 - protein, 654 aa, Nitrobacter winogradskyi
  source 1..654: note = strain: nb-255; note = ABA05523.1 terpene synthase/Squalene cyclase [Nitrobacter winogradskyi Nb-255]
MNSINATAAPIDDNVLGDRIGAATRGLLSLKQSDGHFVFELEADATIPSEYILMRHYLGEPVDTVLEAKIAAYLRRIQGAHGGWPLVHDGPFDMSASVKAYFALKMAGDSIDAPHMARAREAILSRGGAANVNVFTRFLLSFFGELTWRSVPVLPVEIMLLPMWSPFHLNKVSYWARTTMVPLMVLAALKPRARNPRGIGIRELFLEDPATVGTPKRAPHQSPGWFALFTGFDRVLRLIEPLSPKWLRARAMKKAIAFVEERLNGEDGLGAIFPPMVNTVMMYDALGFPPEHPPRAVTRRGIDKLLVVGENEAYCQPCVSPIWDTALSCHALLEAGGPEAVNSAGKCLDWLLLKQELVLKGDWAVKRPDVRPGGWAFQYANGHYPDLDDTAVVVMAMDRVRRNGPNGRYDEAIARGREWIEGMQSRDGGFAAFDADNLEYYLNNIPFSDHAALLDPPTEDVTARCVSMLAQLGETVDSSSSMAAGVEYLRRTQLAEGSWYGRWGLNYIYGTWSVLCALNVAGVDHQDPVIRRAVNWLVSIQNADGGWGEDAVSYRLDYKGFEGAPTTASQTAWALLALMAAGEVENPAVARGIKYLIDTQTKKGLWDEQRYTATGFPRVFYLRYHGYSKFFPLWALARYRNLRSTNSKAVGVGM

SEQ ID NO: 212 - protein, 652 aa, Nitrococcus mobilis
  source 1..652: note = strain: nb-231; note = EAR22397.1 Terpene synthase/Squalene cyclase [Nitrococcus mobilis Nb-231]
MEQQPELISGGVGGVAYPWDLGSQAIEEAILAARAALLAHLHPDGYWCFELEADCTIPAEYIMMMHYTGELEAALELKLARYIRECQLQEGGWPLYYGGAMDISCSVKAYFALKLAGDDPEAAHMRRARKAVLERGGAVNANVFTHIALALFGEIPWRGVPFMPPEILLLPRWFPFHLSKVSYWSRTVMVPLFILAAHKPRARNPRAIHISELFVTDPQLETGYFKARSRLNRLFITLDALGRRIEPFIPRAVRAKALRRAAEWFITRLNGEHGLGAIFPAMVNSYEALELLGYAADHPLRQQVRKGLRDLVVEQADRAYCQPCLSPIWDTALACLALQEADRGSSSAQVRHALDWLQARQLLDTPGDWSEQHPSLPGGGWPFQFRNDHYPDLDDTAIVAWAMQRASDPERYGAAIRRATVWLLGMQSANGGFAAFDSDNTRYYLNEIPFADHGALLDPPTSDVTARVVALLGSLDGEVHDRSALNRAVAFLHREQEAEGCWYGRWGTNYIYGTWSVLTALEQLGYDFNAPWVRKAVIWLKSVQRDDGGWGESNDTYLDHRPQDRQADESTPFQTAWAVLALIAAGECRSPEVWRGVEYLLRHQRPDGLWYCPWFTAPGFPRVFYLKYHGYDAYFPLMALARYRNCVLDNDA

SEQ ID NO: 213 - protein, 659 aa, Nitrosococcus oceani
  source 1..659: note = strain: atcc 19707; note = ABA57818.1 Terpene synthase/Squalene cyclase [Nitrosococcus oceani ATCC 19707]
MTRALRQAPESAGAIGIAAASPATETSGQDTHPREISGAITAARDALLKLQQADGHWCFMLEADCTIPAEYILWTHFTGELEPEIERKLAARLRAKQASHGGWPLYEGGDLDISCSVKVYYALKLVGDDPNAPHMRRAREAILAQGGGARANVFTRLALAMFSQIPWRGVPFIPVEIMLLPRWFPFHLSKVSYWSRTVMVPLAILYSLKAQAQNPRNVHIQELFTVPPEQERHYFPVRSRLNKILLSVERTARLLEPLIPSMLRRRALKKAETWFTERLNGEDGLGGIFPAMVNAHESLILLGYSPDHPWRVQAKKALQNLVIEEKNSASCQPCLSPIWDTGLAALALQETEGGHTTAPVIRALDWLKERQILEQSGDWQVQHPNLKGGGWAFQYNNSYYPDLDDTALVAWSMDQAATPERYGEAIGRACDWLCGMQSRNGGFAAFESDNTHYYLNEIPFADHGALLDPPTADVTARCIVLLGRLNKPQYAETLQRALDYLRREQEPNGSWFGRWGTNYIYGTWSALTALEQANIDPQEGFIRKAVEWLKQVQRLDGGWGEDNYSYFDSSLAGRYQESTPVHTAWALLALMAVGEANSEAVKKGIAYLLQIQQEDGLWDHPAFNAPGFPRVFYLKYHGYDKFFPLWALARYRNHLNRQC

SEQ ID NO: 214 - protein, 670 aa, Nitrosomonas eutropha
  source 1..670: note = strain: c91; note = ABI59752.1 squalene-hopene cyclase [Nitrosomonas eutropha C91]
MSISQAFFRTLIQKSSLSDSSLVSENFPADDVAGNEANEISAVTLDEAITRAYTALLAQQREDGHWCFPLEADCTIPAEYILMMHFMDEVDTVLERKIANFLRTRQVTDGHGGWPLYYGGDFDMSCSVKTYYALKLAGDSPEAAHMVHARNAILERGGAARSNVFTRLLLAMYRQIPWRGVPFVPAEIMLLPRWFPFHLSKVAYWSRTVMVPLSILCTLKAKAINPRNVHVQELFVVDPVKEKNYFPVRTSLNRLLLYVERLASKLEPFIPSFIRRRAVKKAEQWVIERLNGNDGLGAIFPAMVNAYEALTLLGHDRDHPLLQQCRQSLRELLVDEGEEITWCQPCVSPVWDTVLATLALQEDKQADSEPIRRALDWIVPLQILDEPGDWRDSRPNLLGGGWAFQYANPHYPDLDDTAAVAWALIQTGAEDYRVSITRAADWLAGMQSSNGGFAAFDIDNAYYYLNEIPFADHGALLDPPTSDVSARCVGLLALNGEVRHQEAVKRGLDFLFNEQESSGAWFGRWGSNYIYGTWSVLEAFRLARVDKGHQAVQRAIQWLESVQRADGGWGETNDSYLDPQLAGQLEASTSFQTAWAVLGLMAAGEVENTAVRKGIDYLLRTQIATGLWEEPWFTAPGFPRVFYLKYHGYSKYFPLWALNRYRTLSSKSAV

SEQ ID NO: 215 - protein, 665 aa, Nitrosomonas sp.
  source 1..665: note = strain: al212; note = EET32702.1 squalene-hopene cyclase [Nitrosomonas sp. AL212]
MDGSQRISDMSQQPEGIAVSDEISSAYSVSSLNQDEINVDELENKLTQARSAMLSLQKPDGHWCFPLEADCTIPAEYILMMHFMDEIDVILENKIARFIREKQDLTHGGWPLYYGGAFDISCTIKSYYALKLVGDSPDAAHMVRAREAILERGGAAKANVFTRLLLAMYEQIPWSGVPVVPTELMLLPSWFPFHISKVSYWSRTVMIPLSILCTIKARAINPRNVDIRELFIVPPEQEKNYFPQADTWLKRAFMLVERVLSRVEPKLPQAIRQYSIRKAENWTLERLNGECGIGAIFPAMVNAHESLALLGYAYDHPSRVQCRNALRGLLVDEGERAWCQPCTSPVWDTVLTCLALQEDPAADQGPVLKALDWLVDQQVLDEPGDWRDKRPDLLGGGWAFQYANPHYPDLDDTAAVAWALDQSDAQRYQKPLDRAANWLAGMQSRNGGFAAFDIDNTYHYLNEIPFADHGALIDPPTSDVTARCVGLLGKYGKHQREVWRGISFLLREQEKNGSWFGRWGTNYIYGTWSVLEAFQLANFDMQHTSVRRAVKWLESVQRVDGGWGETNDSYLDIQLAGQFPQTSTTFQTAWAVLGLMAAGEVNSKSVRRGINYLLHNQADDHLWEDPWFTAPGFPRVFYLRYHGYSKFFPIWALVRYRALTKERVS

SEQ ID NO: 216 - protein, 653 aa, Nitrosospira multiformis
  source 1..653: note = strain: atcc 25196; note = ABB75845.1 Squalene cyclase [Nitrosospira multiformis ATCC 25196]
MKKFGGMARTSLQAQSPGSNNTPSMDEKMLKAGLEAARGALLAQQREDGHWCFPLEADCTIPAEYILMMHFMDEVDLDLEVRIARFIREKQDVAHGGWPLYYGGEFDLSCSVKAYYALKIVGDSPDAPHMVRARAAILKHGGAARANVFTRLLLAMYDQLPWRGVPFVPVEIILFPKWFPFHTSKVAYWSRTVMVPLSILCSLKARAANPRKVAIRELFTVPPGEERNYFPVRTALNRVFLLIERTLSLLEPFIPQGVRRLALRRAESWIVERLNGDSGLGAIFPAMVNAGEALALLGYPYDHPAREQCRKALRLLLVEEGERTWCQPCVSPVWDTVLTCLAFQEDTEVDQKPIRKALDWLVPCQVLDAPADWQEDHPGLPGGGWAFQYANPHYPDLDDTAAVAWALYQADPKAYQESISRAADWLAGMQSSNGGFAAFDSDNTYYYLNEIPFADHGALLDPPTSDVSARCAGFLALYGQSRHKQALERSLAYLFNEQEASGAWFGRWGSNYIYGTWSVLEAFRLAGIDAGHPAIRRAVHWLKSVQREDGGWGESNDSYLSPQQAGQFHTSTSFHTAWALLALMGAGEWRSHEVHRGIAYLLREQDSDGLWHEPWFTAPGFPRVFYLKYYGYTKYFPVWALTRFHALNRKFPG

SEQ ID NO: 217 - protein, 638 aa, Nostoc punctiforme
  source 1..638: note = strain: pcc 73102; note = ACC84529.1 squalene/oxidosqualene cyclase [Nostoc punctiforme PCC 73102]
MQTQDRVKVNQVAEAIAASQQYLLSIQNPAGYWWAELESNVTITAETVLLHKIWGTDQTRPLHKVEAYLRQEQRQHGGWELFYGDGGELSTSVEAYMALRLLGVPATDPAMIRAQAFILQRGGISKTRIFTKLHLALIGCYNWRGIPSLPPWIMLLPKAFPVNIYEMSSWARSSTVPLLVVCDRKPVFITDPTINLDELYAEGIDRVRWELPQSGDWTDLFLTLDQGFKWAESLNLVPFREEGIKAAEKWILERQEATGDWGGIIPAMLNSMLALRCLDYDRSDPIVERGLQAIDNFAIETDNSYRVQPCVSPVWDTAWVMRALVESGFVPDHPAVVKAGEWLLQKQILDYGDWAVKNRQGKPGAWAFEFENRFYPDVDDSAVVVMALHLAKLPNEKIKQAAIARAVNWIASMQCKPGGWAAFDLDNDQDWLNSIPYGDLKAMIDPNTADVTARVVEMLGACDLSIDSDNLERSLTYLLREQETEGCWFGRWGVNYIYGTSGVLSALALIDPQRHKLSIERGAAWLLGCQNLDGGWGETCRSYDDPSLKGKGDSTASQTAWALIGLLAAGEATGKLAVKAIEQGIGYLMATQQPDGTWFEANFTGTGFPCYFYLKYHLYQQYFPLIALGRYQAAIKES

SEQ ID NO: 218 - protein, 637 aa, Nostoc sp.
  source 1..637: note = strain: pcc 7120 = fachb-418; note = BAB72732.1 squalene-hopene-cyclase [Nostoc sp. PCC 7120 = FACHB-418]
MRTQDRVQVNSIAEAIAASQKYLLSLQNPAGYWWAELESNVTITAEVVLLHKIWGTDKTRPLHKVEAYLRSQQKQHGGWELFYGDGGELSTSVEAYMALKLLGVPATDPAMIQARDFILQRGGISKTRIFTKFHLALIGCYNWRGLPSLPAWVMLLPNQFPVNIYEMSSWARSSTVPLLIVFDQKPVYQVNPTITLDELYAEGVENVRYELPRSGDWTDLFLTLDEGFKLAESFNFIPFREEGIKAAEKWIIERQEATGDWGGIIPAMLNSMLALRSLGYDTNDPIVERGLQALDNFAIETVDCYRVQPCVSPVWDTAWVIRALIDSGIAPDHPAIVKAGEWLLQKQILDYGDWNVKNRQGKPGAWAFEFENRFYPDVDDTAVVVMALHAAKLPNEQLKQKACDRALQWVASMQCKPGGWAAFDLDNDQDWLNSVPYGDLKAMIDPNTADVTARVIEMLGACNLSIDSHNLERALTYLLNEQEAEGCWFGRWGVNYIYGTSGVLSALALINPQKYQRHIQQGATWLVGCQNPDGGWGETCFSYNDPSLKGQGDSTPSQTAWALIGLIAAGEATGNFAHDAIERGINHLVSTQQPDGSWFEAYFTGTGFPCHFYLKYHYYQQYFPLIALGRYQAIKSL

SEQ ID NO: 219 - protein, 654 aa, Afipia carboxidovorans
  source 1..654: note = strain: om5; note = ACI93782.1 squalene-hopene cyclase [Afipia carboxidovorans OM5]
MTSDTASAAALDPRRLATSITRASRALHDVQQPDSHWVFELEADVTIPAEYVMMRHYFAEPVDAEIEAKIAKYLRRMQNDNGGWSLFYGHEFDMSASVKAYYALKMIGDSPDAPHMKKAREAMLARGGASRANVFTRIMLALFGQVSWKAVPMMPVEIMLLPRWFPFHLTKVSYWARTVIVPLLVLMTLKPRAKNPRGIGVRELFLEDPQTVGPTPKAAHQSQLWFTSFDIIDRVLRITDPFFPKGMRKRAIAKAEAFVTERLNGVDGLGAIFPAMVNSIMMYDVLGYPPNDPNRALARESVERLLVIKDDEAYCQPCVSPVWDTALAAHSMLESGEAADIEAAKAGLDWLLPRQVLDLKGDWADKRPDVRPGGWAFQYNNAHYPDLDDTAVVVMAMDRVRRLDGTTKYDEAIARATEWILGLQSENGGWAAFDADNLEYYLNNIPFADHGALLDPPTEDVTARCLSMLAQLGDTLETSEPMRRGVEYLRKTQLPDGSWFGRWGINYVYGTWSVLCALNAVGVPHDDPMIAKAADWLESIQNEDGGWGEDGNSYKLNYKGYERAATTASQTAWATLALMAAGRVDRDATQRGIDNLVQSQEADGFWGEPYYTGGGFPRVFYLRYHGYSKFFPLWAMARYRNLRSSNSRFVGAGM

SEQ ID NO: 220 - protein, 609 aa, Paenibacillus sp.
  source 1..609: note = strain: jdr-2; note = EDS49994.1 squalene/oxidosqualene cyclase [Paenibacillus sp. JDR-2]
MRSELLQLQSADGSWRLCFDSGTMPDSYFIIILRMLGYSQDEALIRQIASRILSRQLPNGTWKIYPDEEDGNLDATAEAYFALLYSGFLTKLDPRMQLAKQFILSKGGLSKIRSLLTQAIFAAAGQASWPKSMRIPLEVFFSDNGIGIDLFSLSGHARVHIVPIIMLANAQFVQHSASMPDLSDLFAGSSKRFENDSPWIAALATLIGSLSLSELLPFESPTPQEKAVQFLFDRLEPDGTLLTYTTATMFMILVLLMLGYSSSSPLIHRMVSGIHSVICANSHVQIASSEVWDTAMLVHALRKAGVNPTSTALENAGAYLRQRQQTQLGDWAIRNPGTPAGGWGFSNVNTLYPDVDDTTAALRAIQPYSSRTPELQADWQRGLNWVLTMRNDNGGWPAFERQGSRLPITFFNFEGAKDIAVDPSTVDLTSRTLQFLGQELGMNAGNSWIESTLRWVLSQQESNGSWYGRWGITYVHGTSAALQGLTAVGIAEDHPAVKKGVDWLLQVQNEDGGWGESCISDKVRRYVPLNFSTPSQTAWALDGLTAALPKPTPALERGVDALLQSLDRHDWTYTYPTGGALPGSVYAHYASNNYIWPLLALSNIWQKYS

SEQ ID NO: 221 - protein, 609 aa, Paenibacillus sp.; identical to SEQ ID NO: 220
  source 1..609: note = strain: jdr-2; note = ACS99948.1 squalene/oxidosqualene cyclase [Paenibacillus sp. JDR-2]

SEQ ID NO: 222 - protein, 642 aa, Paenibacillus sp.
  source 1..642: note = note: oral taxon 786; note = EES74793.1 putative squalene-hopene cyclase [Paenibacillus sp. oral taxon 786 str. D14]
MALSNGEIREEIQRLSEELIQRQEPDGSWRFCFENGITIDACTIILLRTLNVDKEELIRQLHDRIVAAQQPEGCWRWYHDDKEGHLSATVEAYYALLCSGYSRPEDEPIQRAKRYILDRGGIGQARSLFTKAILAATGQRKWPASLSLIPIEILLLPESLPLNFYDFSGYSRVHLVPLLIMAERNFRTRSVRTPDLSELFLDARNGEEDPLTLTPESREPLKLIQSGLAHLVGTPRRIRQAAVNRAEQYMLDRIEGDGTLYTYASCTVLMVFALLALGYEPQHPVIQRAVEGLSQMKFTVDSTGQGGTRYVTIQNSPSTVWDTALISYALQEAGVSSSHPAIQRAADYLRNRQHRRPGDWQIHNPGIVPGGWGFSETNTFVPDVDDTTAALRALSALHGSEPAVLGAWNRGLNWVWSMQNNDGGWPAFEKNTNKEMLTWLAIEGAKSAATDPSEADLTGRTLEYLGNFAKLSVRQDWVARGADWLLSHQEADGSWYGRWGICYIYGTWAALTGLMAVGMPADHPGIAKAANWLIRIQNADGGWGESCRSDQVRRYVPLHASTPSQTAWALDALIAVHDRRAPEIERGVARLIALLHEDDWPSTYPTGAGLPGYFYVHYHSYRYIWPLLALSHYVNKYGDSSP

SEQ ID NO: 223 - protein, 737 aa, Syntrophotalea carbinolica
  source 1..737: note = strain: dsm 2380; note = ABA87701.1 squalene cyclase [Syntrophotalea carbinolica DSM 2380]
MDKIKMKNINQPKFRVFRGGQKAATPCPGTTNERRGALDRGRLSASLKHSREWLLSLQADAGNWVFALEADTTIASEYVMLQRFLGRPLAPELQQRLANYLLSRQLPDGGWPLYAEDGFANISTTVKAYLALKLLGYPTHCDPLVRARQIVLALGGAEKCNVFTRIALALFGQIPWRTTPAMPVEIMLLPRWFYFHLSKISYWARTVVVPLLILYAKRPVCRLEPWEGIPELFVTPPDKLGYLDVCKPGQWRKNVFIWVDRLTRKMVRCVPRRLHNLALRAAETWTREHMQGAGGIGAIFPAMANAVMALRTLGCSPDDADYQRGLKALDDLLIDRCDVPPREDTPVSPCWCTGTSAAPMLDPSPAGSHAQGGDQGICQPCASPIWDTGLALTALLEGGLDARHPAVDRAVRWLLDQQVDVKGDWAQRVPNLEAGGWAFQFENALYPDLDDTSKVLMSLIRAGAMDNPGYRQELSRAINWVIGMQNSDGGWGAFDVDNNYLYLNDIPFADHGALLDPSTADVTGRCIEMLAMAGFGRDFLPIARGVDFLRREQEDFGGWYGRWGVNYIYGTWSALSGLIHAGEDLQAPYIRQAVGWLESVQNPDGGWGETCYSYDDPALAGRGVSTASQTAWALLGLMAAGEVDNLAVRRGIQYLVEEQNRAGGWDERHFTGTGFPRVFYLRYHGYSQYFPLWALGLYERLSSGNPSRQQMVRRAGPAGLHLPVLDRRKKLRRKRKA

SEQ ID NO: 224 - protein, 719 aa, Pelobacter propionicus
  source 1..719: note = strain: dsm 2379; note = ABK98395.1 squalene-hopene cyclase [Pelobacter propionicus DSM 2379]
MKKATRSVFSLLDGGKISDSGSRGDSRHAGSRLDSVTKSAAALLASRQNPDGHWVFDLEADVTIPAEYVMMRCFIGEPLDSDMASRLSAYLLERQLPDGGWPLYAVDGNANISATVKAYFALKLLGHDKYAPHMVSARRMILAQGGAERSNVFTRITLALFGQVPWHTTPAMPIEIMLLPKWFFFHLSKVAYWSRTVIVPLLILYNKQPVCRLGYSEGIAELFSTSPDMLVHLDHFRYRAWRKNAFIVLDRLLKRTMHLVPGRIKRRALEEAERWTRERMKGDGSIGAIYPAMANAVMALKTLGCGDSDPDYLRGLRAIDRLLIHGKPEAGALPADGAGTLFPVLDGASSAAVDLYPASLSDTAKSHAFSFCQPCNSPVWDTALSLTALSEAGGGGYSPERAMEWLFNRQIATQGDWTERCPGLECGGWAFQYENALYPDVDDTAKVLMSLFRAGALERGEYPEKIAKAVRWVLGMQGADGGWGAFDVDNNHFYLNDIPFADHGALLDPSTADLTGRCIEMLGMLGHGPDYPPITRGIEFLREEQEPFGGWFGRWGVNYIYGTWSVLSGLSQAGEDMGRPYVRKAVEWLVSCQNDDGGWGETCASYDDPSLAGSGASTASQTAWALLGLMAAGEADHAAVRAGIAYLADSFADGWDERHFTGTGFPRVFYLRYHGYSLFFPVWALGVYARHREGGKTVQEQVRERGVNGVFDFVMGGSA

SEQ ID NO: 225 - protein, 679 aa, Pelobacter propionicus
  source 1..679: note = strain: dsm 2379; note = ABK98811.1 squalene-hopene cyclase [Pelobacter propionicus DSM 2379]
MNPAKYKISSSLTSLNAEPVEQAPLPAKRTGSKVHRLPPSIWKKMVAEAKSPLDKGIERTRDFFLREQLPDGYWWAELESNVTISAEYVMLFHFLGMVDRERERKLANYILAKQTSEGFWSLWHNGPGDLSTTIEAYFALKLAGYSADHPAMAKARAFVLANGGIIKARVFTKIFLALFGEFAWFGVPSMPIELMLLPDWAYFNMYEFSSWSRATIIPLSVVMSERPVRKLPPRAQVQELFVRPPRPTDYTITREDGLFTWKNFFIGADHLIKVYESSPIRPFKKRAVALAENWILEHQEQSGDWGGIQPAMLNSILALHCLGYANDHPAVAKGLDALANFCIEDDDCIVLQSCVSPVWDTALALVALQEADVPADHPALVKAAQWLLNLEVRRKGDWQVKCPELEPGGWAFEFLNDWYPDVDDSGFVMLSIKNIKVRDRKHREEAIKRGIAWCLGMQSENGGWGAFDRNNTKYLLNKIPFADLEALIDPPTADLTGRMLELMGNFDYPKSHPAAERALAFLKKEQESEGPWWGRWGVNYLYGTWSVLCGLEAIGEDMNQPYIRKAVNWIKSRQNNDGGWGEVCESYFDRSLMGSGPSTASQTGWALLALMAAGEANSRAAAQGVKYLLETQNEDGTWDEDAFTGTGFPKFFMIKYHIYRNCFPLTALGRYRRLTAAKG

SEQ ID NO: 226 - protein, 770 aa, Goniophlebium niponicum
  source 1..770: note = BAI48071.1 germanicene synthase [Goniophlebium niponicum]
MALPFNQDSYKGDDEADVSKGAAKSPPSLEEAIQRSQEFLLAQQFPEGFWFGELEANVTIISHTVILYKLLGIEENFPMYKFERYLRRMQCSHGGWEIAYGIGSYLSATIEAYIALRLLNVPQSDPALQKALRVILDSGGVTKARIFTKICLALLGSFDWRGIPSLPPWLILCPTWFPLSIYEVSSWARGCIVPLLVILDKKPVFKVSPEVSFDELYAEGREHACKIIPISGDWTSKFFITVDRVFKMMERLRVVPFRQWGIREAEKWILERQEESGDYVNIFPAMFYSVMCMKVLGYETTDPVVQRALLGFKGFTIETADECKVQSTVSPIWDTAFIVRALVDSGIPPDHPALQKAGQWLLQKQILKHGDWAFKDRQNPVNQRGFACLQRDSQIETADECRVQSTLSPVWDTAFVVKALVDSGIPPNHPALQKAGQWLLQNQTLTHGDWAFKTQSGHLAAGGWAFQSHNRWYPDADDSAAVMMALDCIELPDEDVKNGAIARGLKWISALQSRNGGWAGYDKNCDQQWINKVPFNDLNGILDVPTADVTARVLEMVGRLSRLGAVGTPYSPRHCTLVESIPHLLLPETIARGLAYLRREQEGEGCWWGKWGVNYIYGTCGALLALSQVAPTTHQEEIARGAKWLAQVQNRCDKQKAAQGPRDGGWGESCFSYDDPALKGQNDASTASQTAWAVQGLLAAGDALGKYEVEAIEQGVQYLLATQRKDGTWHEAHFTGSCFAQHFYVRYHYYAQHFPLSALGLYRTRILQHQ

SEQ ID NO: 227 - protein, 692 aa, Goniophlebium niponicum
  source 1..692: note = BAI48070.1 tirucalladiene synthase [Goniophlebium niponicum]
MLPYNQNSYKEALHGGHAAHNPPTLEEAIKRSQEFLLAHQHPEGFWWGDLECNVTSASHTLILYKILGIADRYPLHKFEKYLRRMQCSHGGWEMSFGDGGYLSATIEAYICLRLLNVPQSDPALQRALKNILARGGVTKARVFTKVCLALLGGFDWAALPSLPPWLMLFPAWFPWNIYEAASWARGCVVPLIVLLEKKPVFQVKPEVSFDELYVEGRAHACKALPFSAHDWVSNIFVAADRAFKLMERFGAVPFRQWSIKEAKKWVLDRQEEMGDFIGYNPPMLYFAVCLKLWGYEVTDPLLQRALLAHKKLTVETEDECWLQSSQSPVWDTALVIPALVESGLPPDHPALQKAGQWLLEKQILKHGDWALKTGGGRMQDDIGGGWAFQFVNSWYPDVDDSAAVVIALNCIKMPDEDVKNGAIARCLKWIAFMQGRNGGWAAFDRDSNQRWMDATPFSDIEAMLDVSTADVTARVLEMVGLMRLKHAAQPANNSLGKAHRHISTESIARGVDYLTKEQEKEGCWWGRWGVNYIYGTRGALMGLSQVAAKTHKKEIARGAAWLVKVQNKKNEKKQGAQDGGWGEACFSYDDPATKGQNSRSTASQTGWAMQGLLAAGEVLGRKYEMEAVEEGVQFLLDTQRKDGSWSEAEFTGGGFPKHYYLKYHYFAQHFPLSALARYRARLLQLSRPKNQA

SEQ ID NO: 228 - protein, 673 aa, Cupriavidus pinatubonensis
  source 1..673: note = strain: jmp134; note = AAZ64302.1 Terpene synthase:Squalene cyclase [Cupriavidus pinatubonensis JMP134]
MSMNEAVLAAPRAAVATAAPALQAPIEALSPLDAGIGHAVDALLAQQNADGHWVYELEADATIPAEYVLMVHYLGETPDLSLEARIARYLRRIQNADGGWPLFHEGRSDISASVKAYFALKMAGDDPQAAHMARAREVILAMGGAETSNVFTRTLLALYGVMPWQAVPMMPVEIMLLPQWFPFHLSKVSYWARTVIVPLLVLNSLRPQARNPRKVGIDELFLGSRDAVRLPPRAPHQHKGWHALFHGADVLLRTAEHVMPRGLRRRAIDAAKAFVRERLNGEDGLGAIFPAMANSVMMFDVLGVPPDDPDRAIARRSIDKLLVVHGDEAYCQPCLSPVWDTALAAHALLEASEPRATAAVTRALDWLRPLQVLDVRGDWTVRRPDVRPGGWAFQYANPHYPDVDDTAVVVAAMHRAARTDHSGRADPNAEATARAIEWIVGMQSANGGWGAFEPENTHLYLNNIPFADHGALLDPPTADVSARCLSMLCQTGATPDKSEPAARALQYLLAEQLPDGSWFGRWGTNYIYGTWSALCALNAAGLGPDAPPLRRAAEWLVAIQNPDGGWGEDGDSYKLEYRGYETAPSVASQTAWALLALMAAGQAAHPAVTRGIDYLLRTQQADGLWHEPRFTAVGFPRVFYLRYHGYARYFPLWALARYRNLERSGNRQVAWGL

SEQ ID NO: 229 - protein, 672 aa, Cupriavidus necator
  source 1..672: note = strain: h16; note = CAJ96989.1 squalene cyclase [Cupriavidus necator H16]
MSMNETAFATAVPRIAPASAGDSPAPRDAAQALDQGIGRAIDALLHQQRPDGHWVYELEADATIPAEYVLMVHYLGEAPDLELEARLARYLRRIQNPDGGWPLFHEGRSDVSASVKAYFALKMAGDDPQAAHMQRARRAVHALGGAEASNVFTRTLLALYGVMPWLAVPMMPVEIMLLPQWFPFHLSKVSYWARTVIVPLLVLNSLRPQARNPRGVGINELFVGNCHTVGLPPRAAHQHAGWYTVFRGLDALLRVAEPLVPRTLRRRAIAAAQAFVRERLNGEDGLGAIFPAMANSVMMFDVLGVPPDDPARALARQSVERLLVEHGDEAYCQPCLSPVWDTALAAHALLETGEARATAAAGRGLDWLRPLQVLDVRGDWAVRRPLVRPGGWAFQYANAYYPDVDDTAVVAAAMNRYMRAHDVPGRYDEAVARAAEWIVGMQGGDGGWGAFEPENTHLYLNNIPFADHGALLDPPTADVSARCLSMLCQIGATPGKSEPAARALRYLLAEQMPDGSWFGRWGTNYIYGTWSALCALNATGLAPEAPEMRRAVAWLEQIQNADGGWGEDGSSYRLDYRGYEPAPSVASQTAWALLALMAAGAAQHAAVARGIDYLLRTQQSGGLWHEPRFTAVGFPRVFYLRYHGYARYFPLWALARYRNLQRGGAHQVPWGL

SEQ ID NO: 230 - protein, 669 aa, Cupriavidus metallidurans
  source 1..669: note = strain: ch34; note = ABF11015.1 squalene-hopene cyclase (plasmid) [Cupriavidus metallidurans CH34]
MNQAATITRPQDETLTTSARRPAQPALPDPLDAGIAHVVESLLAQQQSDGHWVYELEADATIPAEYILMVHYLGETPDLVLEGKIANYLRRIQNADGGWPLFHAGASDISASVKGYFALKMAGDNPEAEHMRRARAAIHAMGGAEASNVFTRTLLALYGVMPWQAVPMMPVEIMLLPEWFPFHLSKVSYWARTVIVPLLVLNSLRPQARNPRKIGIDELFVRPCQATRLPRRAPHQSPLWVGVFRTLDAVVRMAEPLFPRGLRQRAIERAREFTVERLNGEDGLGAIFPAMVNSVLMFDVLGVPESDPNRAIARRSIDKLLVIKDDEAYCQPCLSPVWDTSLAAHALLEVGEPRTIAAAARGLDWLLPLQELELRGDWTVRRPNVRPGGWAFQYANPHYPDVDDTAVVAAAMDRVDKGDRSNRYDEAVSRACEWIVGMQSSNGGWGAFEPENTHLYLNNIPFADHGALLDPPTADVSARCLAMLCQLGQMPANSEPAARALRYLLDEQEADGSWFGRWGTNYIYGTWSALCGLNAAGIGTDAPEMKRAAQWLLSIQNEDGGWGESGDSYKLEYRGYEKAPSTASQTAWAMLGLMAAGAGDHPALVRGVEYLLRTQASHGFWDEPYFTAVGFPRVFYLRYHGYSRFFPLWALARFRNLLRDGNRAISWGL

SEQ ID NO: 231 - protein, 661 aa, Cupriavidus metallidurans
  source 1..661: note = strain: ch34; note = ABF11268.1 Squalene cyclase (plasmid) [Cupriavidus metallidurans CH34]
MTRKTIPASELDAAIVRARDALLDRQHPDGHWCFELECDATITAEYILMMHFVDEIDTALQARMAKYLRAVQRLDGHGAWDLYFGGDLDISCSVKAYFALKAAGDPPDAPHMVRAREAILARGGAAKSNVFTRILLATFGEIPWRGTPFMPVEFVLFPRWAPIHMDKVAYWARTTMVPLLVLCSIRAAAKNPLGVHVQELFVTPPELEREYFPRKRGLQQAFLVADRVVRHLEPLIPRALRRRAIQRAVEWSEARMNGEDGFGGIFPPMVYSYEMMVLLDYPEDHPLRVECKAALKKLVVHRDDGSSYCQPCLSPVWDTAWSVMALEQAPSDARTETAIARAYDWLTDRQVLDLRGDWENNAAPSTPPGGWAFQYENPYYPDIDDSAVVLAMLHARGKRTGQPGRYEMPVARCLDWIIGLQSRNGGFGAFDANCDRDFLNAIPFADHGALLDPPTEDVSGRVLLALGITERPQDATARERCIQYLRDTQQPDGSWWGRWGTNYIYGTWSVLAGLGLAGVDRKLPMVRNGLQWLRGKQNADGGWGETNDSYARPELAGKHEDGSMAEQTAWAMLGQMAVGEGDADSVHRGAAYLLDAQNEDGFWMHPYHNAPGFPRIFHLKYHGYTAYFPLWALGRYRRLAAARASAMQTAKAESAESMTAH

SEQ ID NO: 232 - protein, 647 aa, Rhizobium sp.
  source 1..647: note = sp|P55348.1|SQHC_SINFN RecName: Full=Probable squalene--hopene cyclase - AltName: Full=Squalene--hopanol cyclase
MNKHSGNRTAIDPAALEMSIASATEALLAYRHADGHWAFELEADSTIPSEYILLRHYLAEPIDVVLEAKIGNYLRRTQGAHGGWPLVHDGPFDMSASVKSYFALKMIGDSVDAAHMVKAREAIRARGGAANSNVLTRFLLALYGVVSWRAVPVLPIEIVLLPIWSPFHLYKISYWARTTIVPLMVLAVLKPRAKNPKGVGIEELFLQDTKSVGMNPKAPHQSWGWFLLFRGIDGILRVIEPHLPKKLRERAIASALAFTEERLNGEDGMGAIYPSMANIVMMYDALGKDDHFPPRAIARRAIDKLLVIGEEEAYCQPCLSPVWDTALTCHALQEVGGANAVAKAKQGLDWLKPRQVLDVKGDWAVKAPNIRPGGWPFQYNNAHYPDLDDTAVVVMAMDRAQRHAGSKEYATAIARGREWIEGMQSRDGGWAAFDVNNLEYYLNNLPFADHGALLDPPTEDVTARCVSMLAQVGEFTQRSKAVAEGIAYLRRTQHAEGSWYGRWGLNYIYGTWSVLCALNAAGIDHQDPMIRKAVEWLVSIQSWDGGWGEDAISYRLDYSGYEQAPSTSSQTAWALLGLMAAGEVEHPAVARGVNYLKNAQTENGLWDEQRYTATGFPRVFYLRYHGYSKFFPLWALARYRNLRSTNV

SEQ ID NO: 233 - protein, 661 aa, Rhodopirellula baltica
  source 1..661: note = strain: sh 1; note = CAD74517.1 probable squalene-hopene cyclase [Rhodopirellula baltica SH 1]
MMAVVENSVSEVLDRRELRGTLDLLRGELLAQRTKDGHWTGELSASALSTATAISAMSAAVRSGKLAGADKAALLEQIQSGRRWLADQQNDDGGFGDTDRSHSNIATSYLVLAAWTLSDQVTGETTDANAISRLRNWIQLAGELDGLRRRYGKDKTFVVPILTNMAIAGLVPWKKVSALPFEAAVVPQSMYRFVGMPVVSYAVPALVAIGQVKFLEGGGCLPPWSLVRRAAIEPSMKVLRSMQPSSGGYLEATPLTAFVVMSLSASGRADHEVTQNGLRFLRDSMLPDGSWPIDTNLANWATSLATTALTMDPDDDRSWSTNELIQWQRGCQYQERHPFTGADPGGWGWTDLTGSVPDADDTPGAIISLRMQATTRPDPLCDDYSRDWPASDSSGSVSANALDTWKACDRGVDWLLGLQNRDGGWPTFCRGWGKLPFDRSSNDLTAHALRAIACLPKRESAKRSRAVQRGLRFLRKNQQADGSWLPLWFGNQDRPEEDNPIYGTSRVLVDVSPALGHDAISRGLYYLINSQNSDGGWGGGESVRETFGLPEGFISSVEETALAVEALVSWWGRIPGNEGGQAAENDIPDGSPWDASMRSALRAAILSGTRWLIDAVQRERHQVAWPIGFYFAKLWYYERLYPLVYTTAALGRVMQRDELLR

SEQ ID NO: 234 - protein, 653 aa, Rhodopseudomonas palustris
  source 1..653: note = strain: tie-1; note = ACF02757.1 squalene-hopene cyclase [Rhodopseudomonas palustris TIE-1]
MDSGSYTTGVERNALEASIDAARSALLNYRRDDGHWVFELEADCTIPAEYVLLRHYLGEPVDAELEAKIAVYLRRIQGAHGGWPLVHDGDFDMSASVKGYFALKMIGDSIDAPHMVRAREAIRSRGGAIHSNVFTRFLLTLYGVTTWRAVPVLPVEIMLLPSWSPFTLTKISYWARTTMVPLLVLCALKPQAKNPKGVGIDELFLQDPKTIGMPVKAPHQNWALFKLFGSIDAVLRVIEPVMPKGIRKRAIDKALAFIEERLNGEDGMGAIFPPMANAVMMYEALGYPEDYPPRASQRRGIDLLLVDRGDEAYCQPCVSPVWDTALASHAVLEADGHEGAKSVRPALDWLLPRQVLDVKGDWAVKAPNVRPGGWAFQYNNAHYPDLDDTAVVVMALDRARKDQPNPAYDAAIARAREWIEGMQSDDGGWGAFDINNTEYYLNNIPFSDHGAMLDPPTEDVTARCVSMLAQLGETMDSSPALARAVGYLRDTQLAEGSWYGRWGMNYIYGTWSVLCALNAAGVPHADPMIRKAVAWLESVQNRDGGWGEDAVSYRLDYRGYESAPSTASQTAWALLALMAAGEVDHPAVARGIEYLKSTQTEKGLWDEQRYTATGFPRVFYLRYHGYSKFFPLWALARYRNLQATNSKVVGVGM

SEQ ID NO: 235 - protein, 654 aa, Rhodopseudomonas pseudopalustris
  source 1..654: note = CAA71101.1 squalene-hopene cyclase [Rhodopseudomonas pseudopalustris]
MDSGTFNPGGERGNTLDASIDAARAALLGYRRDDGHWVFELEADCTIPAEYVLLRHYLGEPIDAALEAKIAVYLRRTQGAHGGWPLVYDGEFDMSATVKGYFALKMIGDSIDAPHMAKAREAILSRGGAVHANVFTRFLLAMFGILTWRAVPVLPVEIMLLPMWSPFHLNKISYWARTTIVPLMVLAALKPRAVNRLGVGLDELFLQDPKSIGMPARGPHQNRGLFALFGAIDAVLRVIEPLIPKKLRKHAIDRAVAFVEERLNGEDGLGAIYPPMANTVMMYKVLGYPEDHPPRAITRRGIDLLLVIGEEEAYCQPCVSPIWDTSLTCHALLEAGGAEAAQPVREGLDWLLPKQVLDLKGDWAVKAPNVRPGGWAFQYNNAHYPDLDDTAVVVMALDRARRDQPSAAYDNAIARGREWIEGMQSDDGGWAAFDVNNTEYYLNNIPFSDHGAMLDPPTEDVTARCVSMLAQLGETEQTSKAVARGVAYLRKTQLPDGSWYGRWGMNYIYGTWAVLCALNAAGVDHQDPAIRKAVAWLASIQNADGGWGEDGVSYRLDYRGYETAPSTASQTAWALLSIMAAGEVDHPAVARGIEYLKGTQTEKGLWDEQRHTATGFPRVFYLRYHGYSKFFPLWGLARYRNLRATNSKVVGVGM

SEQ ID NO: 236 - protein, 654 aa, Rhodopseudomonas palustris
  source 1..654: note = strain: haa2; note = ABD06434.1 Squalene cyclase [Rhodopseudomonas palustris HaA2]
MTSGTTILGAERGRTLDASIDAARAALLGYRRDDGHWVFELEADCTIPAEYVLLRHYLGEPVDAALEAKIAVYLRRTQGAHGGWPLVHDGEFDVSATVKAYFALKMIGDSIDAPHMAKAREAILARGGAIHVNVFTRFLLSMFGILTWRSVPVLPVEIMLLPMWAPFHLNKISYWARTTIVPLMVLAALKPRAVNKLDIGLDELFLQDPQSIGMPAKAPHQSWGLFTLFGSIDAVLRVIEPLIPKKLRSYAIGRAVAFIEERLNGEDGLGAIYPPMANTVMMYKVLGYGEDHPPRAITRRGIDLLLVVGEEEAYCQPCVSPIWDTSLTCHALLEAGGAEAALPVRKGLDWLIPKQVLDLKGDWAVKAPNVRPGGWAFQYNNAHYPDLDDTAVVVMALDRARRDQPSAAYDNAIARGREWIEGMQSDDGGWAAFDVNNTEYYLNNIPFSDHGALLDPPTEDVTARCVSMLAQLGETAETSSALARGVAYLRKTQLAEGSWYGRWGLNYIYGTWSVLCALNAAGVAHQDPAMRKAVAWLASIQNADGGWGEDAVSYRLDYRGYESAPSTASQTAWALLALMAAGEVDHPAVARGVEYLKGTQTEKGVWDEQRYTATGFPRVFYLRYHGYSKFFPLWALARYRNLRATNSKVVGVGM

SEQ ID NO: 237 - protein, 654 aa, Rhodopseudomonas palustris
  source 1..654: note = strain: bisb18; note = ABD87279.1 Squalene cyclase [Rhodopseudomonas palustris BisB18]
MESGNNKQPAAAIGALDASIESATNALLGYRQPDGHWVFELEADCTIPAEYVLLRHYLGEPVDAALEAKIANYLRRVQGAHGGWPLVHDGGFDMSASVKGYFALKMIGDDIDAPHMAKAREAIRSRGGAIHSNVFTRFLLSMFGITTWRSVPVLPVEIMLLPMWSPFHLNKISYWARTTIVPLMVLAALKPRAVNRLDIGLDELFLQDPKSIKMPAKAPHQSWALFKLFAGIDAVLRTIEPLFPKRLRDHAIKLAVDFVEERLNGEDGLGAIYPPMANTVMMYKVLGFPEDHPPRAITRRGIDKLLVIGEDEAYCQPCVSPVWDTALTCHALLEVGGEAAVPPAKRGMDWLLPKQVLDLKGDWAVKRPNLRPGGWAFQYNNAHYPDLDDTAVVVMAMDRSRRATGSREYDEAIARAREWIEGMQSDDGGWAAFDVNNLEYYLNNIPFSDHGAMLDPPTEDVTARCVSMLSQLGETAASSKAVADGVEYLRRTQLPDGSWYGRWGLNYIYGTWSVLCALNAAGVDHQDPVIRKAVTWLASVQNPDGGWGEGAESYRLNYTRYEQAPTTASQTSWALLGLMAAGEVDSPVVARGVEYLKSTQTGKGLWDEQRYTATGFPRVFYLRYHGYAKFFPLWALARYRNLRSTNSKVVGVGM

SEQ ID NO: 238 - protein, 656 aa, Rhodospirillum rubrum
  source 1..656: note = strain: atcc 11170; note = ABC20867.1 terpene synthase - squalene cyclase [Rhodospirillum rubrum ATCC 11170]
MDATAPLRDPGAPSAENCSVDRRELDDVIGESCRWLGERQNQDGHWVFELEADATIPAEYILLNHFLDEIDDAREARIASYLRAIQGKHGGWPLFHDGDFDMSATVKAYYALKLTGDGVDEPHMVRARQAILEHGGAERTNVFTRFTLAMFDQVPWRACPVTPVEALLLPRFAPFHWSKVSYWSRTVMTPLMILYSRRARAVNPRGIGVRELFRRDPEVIRDWLKNPTGHWIGDALIQIDKVLRVIEPAIHWAFRDRAEKWALDFIEERLNGRDGLGGIYPAIANTLMAYHTLGYAKDHPGYRIAREAVDGLCTPHAKGEYVQPCLSPVWDTCLASHAIQEAGQSAGDRAVDQSNAWLRERQVLDVVGDWKSNRGHLRPGGWAFQYNNPHYPDVDDTAVVVMALARSKEDEANREAIARAEEWIIGMQSSNGGWGAFDAENEHDFLNHVPFADHGALLDPPTVDVSARCLGMLAQLGRPKTDPVVARGLDYLWREQEADGSWFGRWGTNYIYGTWSALNAFNAVEWDMTDPRICKAVDWLKSRQRDDGGWGEDCATYWKERRSVSKASTPSQTAWAVLGLMAAGEVDSPEVERGIRYLLEAPRDGGKWEEELYNAVGFPRIFYLRYHGYSAYFPLWALARYRNLTSGNCKRTIHGM

SEQ ID NO: 239 - protein, 554 aa, Rubrobacter xylanophilus
  source 1..554: note = strain: dsm 9941; note = ABG05671.1 Prenyltransferase/squalene oxidase [Rubrobacter xylanophilus DSM 9941]
MGRQTRNLTRREPAAEAEERGFRLLDAHRRADSSWVGELSSSALATAMSALALRLLGHPAESGPVAGGLAWLAATRNPDGGWGDAPGEPSNMNATSIAAAALARCAPRRYREEVAGGRRWVEEHGGFAALNDPRTTTLSGPGRTLWALAGLVPPERVRKLPTEMILLPRRIRRTVSTTFPAFLSLSLLHERFRPSPRWRRPLRRRAEREALAWLRRAQGPNGSYEESAFLTSLIAAALTAAGAEGGDIVRRALPFVLRSRRPDGSWPIDRDLENFDTTQAILAHHEAGRPLREAGRVREWLLDNQFRRPFFPTSSPPGGWAWAYPAGWPDTDDTACALRSLRLLGVPAGHPSIRLGLRWLYRMQNRDGSWPTFVRGSRMPFDHGCPYITSQVLSALALMGPEARRGAPLRRALAYLRRAQRPDGSLGSLWFRPHTRGTAAAVEAFSDLGLSGDPLVGRAARWLAEHQNPDGGWGDGHGAPSTAEETAWASAALLRLGGGEAARKGVRWLVEHQDPGGWKPAVIGLYYASLSYSDTFYALSYPLVALARHRRLSR

SEQ ID NO: 240 - protein, 640 aa, Saccharomonospora viridis
  source 1..640: note = strain: dsm 43017; note = ACU97316.1 squalene-hopene cyclase [Saccharomonospora viridis DSM 43017]
MTDVLTRELSPNSTRDRVRSCVSSARQYLLSLQHEEGWWKGELDTNVTMEAEDLLLRQFLGISDEQVTQETARWIRSCQREDGTWATFHGGPPDLSTTVEAYVALRLAGDAMDAAHLRKAREYILDSGGIESTRVFTRIWLALFGEWPWSRLPVLPPEMMLLPDWFPLNIYDWASWARQTVVPLTIVGSLRPTRDLGFSVRELRTGIQRRDLESPLSWAGVFHGLDSVLHRLEKLPLKPLRKVALARAEQWILDRQESDGGWGGIQPPWVYSILALHLRGYPLDHPVLRKALDGLDGFTIRHRTENGWIRKLEACQSPVWDTALAMTALLDSGTPPNDPALVRAADWILRQEIRVSGDWRVRRPALEPSGWAFEFANDHYPDTDDTAEVVLGLQRVRHPEPHRVNAAVERATAWLVGMQSSDGGWGAFDADNTRTLCEKLPFCDFGAVIDPPSADVTAHIVEMLAARGMADSESARRGVRWLLEHQEVDGSWFGRWGANHVYGTGAVVPALVACGISPQHEAVRAAVQWLVAHQNADGGWGEDLRSYVDRTWVGRGTSTPSQTAWALLALLAAGERGEVVRRGVEWLMAAQRPDGGWDEPQYTGTGFPGDFYISYHMYRIVFPLTALGRYLGRGGDVGTG

SEQ ID NO: 241 - protein, 636 aa, Saccharopolyspora erythraea
  source 1..636: note = strain: nrrl 2338; note = CAM03596.1 squalene--hopene cyclase [Saccharopolyspora erythraea NRRL 2338]
MTEEVLQRTAAPAEVLAAAREHLLSLQHERGWWKGELETNVTMDVEDLLLRRFLGILTTAETEQAARWIRSRQRADGTWAQFHGGPGDLSTTVEAYVGLKLAGDDVDSEHMAAARAWILERGGIEETRVFTRIWLALFGEWSWDDLPAMPPELVLLPPWVPLNLADWGCWARQTIVPLTVVCTLRPRRDLGVGLAELRSGRRRRKVPSPSWAGAFQVLDGALHGYQRHPLRGLREHAMRRAAEWIVARQEADGSWGGIQPPWVYSLLALHLLGYPLDHPVLRQGLAGLERFLIREETPEGTVRRLEACQSPVWDTVLSMQALRDAGLAADHPALRRAADFVLAEEIRVKGDWSVRRPDLAPGGWAFEFDNDGYPDIDDTAEVVLALNRVDHERPGAVNAAIDRGVRWMSGMQSADGGWGAFDADNTRELVNELPFCDFGAVIDPPSADVTAHVVEALCVLGRGDGEAVRRGVRWLLDHQELDGSWFGRWGANHVYGTGAAVPALVRAGLRRDHLALRRAVRWLEVHQNDDGGWGEDLRSYDDPVWVGRGRSTASQTAWALLALLAVDLHDTDAVRRGVGFLAETQRPDGTWDEPQFTGTGFPGDFYINYHLYRLVFPVTALGRYEQARREQSGGSG

SEQ ID NO: 242 - protein, 656 aa, Schizosaccharomyces japonicus
  source 1..656: note = strain: yfs275; note = EEB08219.1 squalene-hopene cyclase [Schizosaccharomyces japonicus yFS275]
MKTDGNTTLDTTISMEELERTVKSAYEALAKDQQDDGHWIYELEADVTIPAQFILLEHTLDKIDEELEQKIANYLRRCQSREHWGWPVYYGGEFNISASVQAYFALKMTGEDINAPHMVRAREAILAHGGPEYANVFTRIQLSLFGEASWLATPFMPVEIMLLPRWMYFSIWNMSYWSRTTVAPLLIVADLKPKAINPRNVHIPELFPTPPDKVKTWIHGPFRSKWGHVFKFIDTAIRPFTRFVPSFLHKKAYKAALDFIEPRLNGVDGLGAIYPPMSYSAVMYRALGIPDDDPRAATNWEALKGLLVIKEREAYCQACVSPVWDTALSGHALMEASFGPDGINADRTEKLIDRAAHWLRAHQVLNVVGDWAINNPNLQPGGWAFQYGNDYYPDVDDTAVAAMLLHRQNLPENEEALDRARKWIIGMQSSNGGWGAFDIDNDKQILNDIPFADHGALLDPPTADVSARCISLLAELGHPEDRPVIERGIKYLRKEQEEDGSWFGRWGTNYIYGAWSVLCAFNASGVPHDDPSVLKCVNFLKSVQREDGGWGESCETYEGSAHGVYTESLPSQTAWAVLGLMASGRRTDPAVKRGIVWLIQHQQDNGEWAEEPFNAVGFPRMFYLHYLGYKQFFPLLALARYRHMEKSGTNNVSFAF

SEQ ID NO: 243 - protein, 617 aa, Sphaerobacter thermophilus
  source 1..617: note = strain: dsm 20745; note = ACZ39437.1 squalene-hopene cyclase [Sphaerobacter thermophilus DSM 20745]
MDPALSRAVDWLLEHQDPAGWWCGEFETNVTITAEHILLLRFLGLDPSPLRDAVTRYLLGQQREDGSWALYYEGPADLSTSIEAYAALKVLGLDPTSEPMRRALQVIHDLGGVAQARVFTRIWLAMFGQYPWDGVPSMPPELIWLPPSAPFNLYDFACWARATITPLLIILARRPVRPLGCDLGELVLPGSEHLLTRVPGSGPFWWGDKVLKRYDHLVRHPGRDRACQRIVEWIIARQEADGSWGGIQSAWVMSLIALHLEGLPLDHPVMRAGLAGFDRVALEDERGWRLQASTSPVWDTAWAVLALRRAGLPREHPRLALAVDWLLQEQIPGGGDWQVRTGTIPGGGWAFEFDNDHYPDIDDTAVVVLALLEAGHEDRVRNAVERAARWILAMRSTDGGWGAFDRDNAREVIHRLPIADFGTLIDPPSEDVTAHVLEMLARLSFPSTDPVVARGLEFLQQTQRPDGAWFGRWGVNYIYGTWCAVSALTAFADTDATARAMVPRAVAWLLDRQNADGGWGETCGSYEDPNLAGVGRSTPSQTAWAVLALQAAGLGQHPACRRGLDFLRERQVGGTWEEREHTGTGFPGDFFINYHLYRHVFPTMALAGAATGMDSPR

SEQ ID NO: 244 - protein, 583 aa, Streptomyces albus
  source 1..583: note = strain: j1074; note = ZP_04706071.1 squalene-hopene cyclase - partial [Streptomyces albus J1074]
FLGIRDEATTRSAALFIRGEQREDGTWATFHGGPPDLSTTVEAYVALRLAGDSPDAPHMTRAAHWVRSQGGIAEARVFTRIWLALFGWWPWDRLPELPPELIFLPPWAPLNIYDFGCWARQTIVPLTVVSAKRPVRPAPFPLDELHTDPADPAPRARFAPLASWNGAFQRLDRALHAYRKVAPRALRRAAMATAGRWIVERQENDGCWGGIQPPAVYSMIALHLLGYDLGHPVMRAGLESLDRFTLTREDGSRMVEACQSPVWDTCLATIALADAGVPADHPQLVRAADWMLDEQIERPGDWSVRRPHLAPGGWAFEFHNDNYPDIDDTAEVVLALRRVRHPDTARMERAISLGVRWNLGMQSKNGAWGAFDVDNTSSLPNRLPFCDFGEVVDPPSADVTAHVVEMLAAEGLAADPRTRRAVDWLLAEQEPSGAWFGRWGVNYLYGTGSAVPALVDAGLPTTHPAIRRAVAWLESVQNDDGGWGEDLRSYREQGRMARGASTASQTGWALMALLAAGERESRAARRGVTFLAETQHEDGSWEEPYYTGTGFPWDFSINYHLYRQVFPLTALGRYTRGAAPEGA

SEQ ID NO: 245 - protein, 666 aa, Streptomyces avermitilis
  source 1..666: note = strain: ma-4680 = nbrc 14893; note = BAC69361.1 squalene-hopene cyclase [Streptomyces avermitilis MA-4680 = NBRC 14893]
MTATTDGSTGALPPRAASASEPHDTIPQAAGSVGIQDAAARATQRATDFLLSRQDAEGWWKGDLETNVTMDAEDLLLRQFLGIQDEKTTRAAGLFIRGEQRADGTWATFYGGPGDLSATIEAYVALRLAGDGPDEPHMAKASAWIRERGGIASARVFTRIWLALFGWWKWDDLPELPPELIYFPKWMPLNIYDFGCWARQTIVPLTVVSAKRPVRPAPFPLDELHADANDPNPAKPLAPMVSWDGLFQRLDVALHTYRKVAPRRLRKAAMNTAARWIIERQENDGCWGGIQPPAVYSVIALYLLGYDLEHPVMREGLASLDRFAVWRDDGARMIEACQSPVWDTCLATIALADAGVPADHPQLVRAADWMLGEEIVRPGDWAVKRPQLPPGGWAFEFHNDNYPDIDDTAEVVLALRRVKHHDPERLDNAIRRGVRWNLGMQSKDGGWGAFDVDNTSPFPNRLPFCDFGEVIDPPSADVTAHVVEMLAFEGLSHDPRTRRGIQWLLSAQEANGSWFGRWGVNYVYGTGSVVPALVAAGLPASHPAIRRAVTWLETVQNDDGGWGEDLRSYPEAAEWSGKGASTASQTGWALLALLAAGERESKAVERGIEWLAQTQRPDGSWDEPYFTGTGFPWDFSINYHLYRQVFPLTALGRYVNGEPLVEVKGG

SEQ ID NO: 246 - protein, 639 aa, Acaryochloris marina
  source 1..639: note = strain: mbic11017; note = ABW29816.1 squalene-hopene cyclase [Acaryochloris marina MBIC11017]
MAVRDRVNPKTLEAAIAASQSYLLTQQDETGYWWAELESNVSITSEVVLLHKIWGTDRSRPLEKVETYLRSQQRDHGGWELYFDDGGEISVSVEAYMALKLLGVPMEDPAMVRARQFILEHGGISRTRVFTKLHLALIGCYEWRGIPSLPPWVMLLPEQFPFTIYEMSSWARGSTVPLLIVMDREPVYAVEAGFNLDELYVEGRHRAQFDLPLSNEWTDAFIYLDGLFKFAESTNLVPFREEGIRAAERWILERQEATGDWGGIIPAMLNSLLGLKALDYDVHDPIIERGMAALDAFALETEDQYWIQPCISPVWDTALVVRGLAESGLAPDHPALVKAGEWLLNKQILDYGDWSVKNPGGLPGGWAFEFDNRFYPDVDDTAVVVMALNEVQLPDEQAKDAAIARAVNWIATMQCRPGGWAAFDINNDQDWLNALPYGDLKAMIDPNTADVTARVLEMIGRCHQTTGKNSVDRALRYLRTEQEPEGCWFGRWGVNYIYGTSGVLAALALIDPQGWQSQIQQAAAWLVSCQNTDGGWGETCASYDNPKLKGQGPSTASQTAWAIMGLLSAGEATSVYAEAAIERGVNYLTTTQKMDGTWDEDYFTGTGFPGHFYLKYHLYQQHFPLTALGRYQAMLQQKS

SEQ ID NO: 247 - protein, 666 aa, Streptomyces roseosporus
  source 1..666: note = strain: nrrl 15998; note = ZP_04697579.1 putative squalene-hopene cyclase [Streptomyces roseosporus NRRL 15998]
MTATTDGSTGAANITGAPADDPTDTRTAANDVTDIARRAAERSVEHLLGRQDEQGWWKGDLATNVTMDAEDLLLRQFLGIQDPATTRAAALFIRGEQLGDGTWNTFYGGPGDLSATIEAYVALRLAGDRPDEPHMARASGWIRDQGGIAAARVFTRIWLALFGWWKWDDLPELPPELMFFPKWVPLNIYDFGCWARQTIVPLTIVSAKRPVRPAPFALDELHTDPDHPNPPRKLAPPTSWDGLFQRLDKGLHLYHKVAPRPLRRIAMNVAARWIIERQENDGCWGGIQPPAVYSVIALHLLGYDLDHPVMKAGLASLDRFAVHREDGARMIEACQSPVWDTCLATIALADAGLRPDHPALVKAADWMLAEEITRPGDWSVRKPELAPGGWAFEFHNDNYPDIDDTAEVVLALRRVRHPDPARLEAAIARGVRWNLGMQSRNGAWGAFDADNTSPFPNRLPFCDFGEVIDPPSADVTGHVVEMLAVEGLANHPRTREGIEWLLAEQEACGAWFGRWGVNYVYGTGSVVPALITAGLPAGHPAIRRAVDWLESVQNDDGGWGEDLRSYQEEKWIGHGESTASQTAWALLALLAAGRRDTASVTRGVTWLTEAQQADGSWDEPYFTGTGFPWDFSINYHLYRQVFPLTALGRYVHGDPFADRTDAAEGV

SEQ ID NO: 248 - protein, 670 aa, Streptomyces flavogriseus
  source 1..670: note = strain: atcc 33331; note = EEW70811.1 squalene-hopene cyclase [Streptomyces flavogriseus ATCC 33331]
MTATTDGSTGAANLRAAAASDPTESTSAAPDMMAVARHAAERSVEHLLGRQDEQGWWKGDLATNVTMDAEDLLLRQFLGIQDPETVKAAARFIRGEQLGDGTWNTFYEGPPDLSATVEAYVALRLAGDRPDDPHMIRAAGWVREQGGIAESRVFTRIWLALFGWWKWDDLPELPPELMFFPKWVPLNIYDFGCWARQTIVPLTIVSAKRPVRPAPFALDELHTDPACPNPSRPTAPAASWDGVFQRLDKALHLYHKVAPRRLRRIAMNEAARWIIERQENDGCWGGIQPPAVYSVIALHLLGYDLDHPVMRAGLESLDRFAVWREDGARMIEACQSPVWDTCLATIALADAGVSPDHPALVRAADWMLGEEIVRPGDWAVRKPGLAPGGWAFEFHNVNYPDIDDTAEVALALRRVRHPDPARVDAAIERGVRWNLGMQSRNGAWGAFDADNTSPFPNRLPFCDFGEVIDPPSADVTGHVVEMLAVEGRAHDPRTRRGVEWLLAEQEASGAWFGRWGVNYIYGTGSVVPALIAAGLPAAHPSVRRAVDWLRSVQNDDGGWGEDLRSYREEKWIGHGSSTASQTGWALLALLAAGERETRSVERGVAWLAATQQADGSWDEPHFTGTGFPWDFSINYHLYRQVFPLTALGRYVYGDPFATATAIGAGTGKGA

SEQ ID NO: 249 - protein, 686 aa, Streptomyces ghanaensis
  source 1..686: note = strain: atcc 14672; note = ZP_04684415.1 squalene-hopene cyclase [Streptomyces ghanaensis ATCC 14672]
MTATTDGSTGAALPPRVTAASDTDTDIPVAAGVPDIAARAMRRATDFLLSRQSDQGWWKGDLETNVTMDAEDLLLRQFLGIRDEGTTRAAALFIRGEQREDGTWATFHGGPGDLSATIEAYVALRLAGDPPDAPHLARASAWIREQGGIAASRVFTRIWLALFGWWKWEDLPELPPELIWFPAWVPLNIYDFGCWARQTIVPLTIVSAERPVRPAPFPLDELHTDPARPNPPRALAPVTGWDGAFQRLDKALHVLRGAVPRRLRRAAMNTAARWIIERQENDGCWGGIQPPAVYSIIALHLLGYDLNHPVMRAGLESLDRFAVWREDGARMIEACQSPVWDTCLATIALADAGLPADHPQLVKAADWMLGEQIVRPGDWSVRRPHLPPGGWAFEFHNDNYPDIDDTAEVVLALRRVAHHDPERVDNAIGRGVRWNLGMQSRNGAWGAFDVDNTSPFPNRLPFCDFGEVIDPPSADVTAHVVEMLAAEGLAHDPRTRRGVQWLLAEQEPNGSWFGRWGVNYLYGTGSVVPALTAAGISGSHPAIRRAVAWLESVQNDDGGWGEDLRSYRDARGWSGRGASTASQTAWALMALLAAGERESRAVERGVEWLAATQHEDGSWDEPYFTGTGFPWDFSINYHLYRQVFPLTALGRYVNGEPLAGKPRAAGAATAREDTGQEQSLAEAKGS

SEQ ID NO: 250 - protein, 680 aa, Streptomyces griseoflavus
  source 1..680: note = strain: tu4000; note = ZP_05537325.1 squalene-hopene cyclase [Streptomyces griseoflavus Tu4000]
MTATTDGSTGAALPPRVTAASENDTDIPEAAGVPDIAAHAMRRATDFLLSRQDDQGWWKGDLETNVTMDAEDLLLRQFLGIRDEDTTRAAALFIRGEQREDGTWATFHGGPGELSTTIEAYVALRLAGDPPEAPHMARASAWIRERGGIAAARVFTRIWLALFGWWKWEDLPELPPELIWFPSWVPLNIYDFGCWARQTIVPLTIVSAKRPVRPAPFPLDELHTDPRRPRPPRPHAPPNTWDGAFQRLDRALHALRRAVPRRVRQAAMNAAARWIIERQENDGCWGGIQPPAVYSVIALHLLGYDLRHPVMRAGLESLDRFAVWREDGARMIEACQSPVWDTCLAAIALADAGLPADHPSLVKAADWMLGEQIVRPGDWSVRRPHLPPGGWAFEFHNDNYPDIDDTAEVVLALRRVRHHDPERMDSAIGRGVRWSLGMQSKNGAWGAFDVDNTSPFPNRLPFCDFGEVIDPPSADVTAHVVEMLAVEGLAHDPRTRRGIQWLLAEQEPDGSWFGRWGVNYLYGTGSVVPALAAAGIPGSHPAIRRAVAWLEKVQNDDGGWGEDLRSYRHVREWSGRGASTASQTAWALMALLAAGERDSGAVERGVAWLAATQREDGSWDEPYFTGTGFPWDFSINYHLYRQVFPLTALGRYVHGEPFSKKQTAARNGSAQPLAGVKGSR

SEQ ID NO: 251 - protein, 630 aa, Streptomyces hygroscopicus
  source 1..630: note = strain: atcc 53653; note = ZP_05513599.1 putative squalene-hopene cyclase [Streptomyces hygroscopicus ATCC 53653]
MLARRATDRAVRHLLSRQDEQGWWKGDLETNVTMDAEDLMLRHFLGIQNPDVLDAAGRYIRSQQAADGTWATFHGGPPELSATVEAYVALRLAGDPPDAPHMAAASAWVRNNGGVASSRVFTRIWLALFGWWRWEDLPELPPEIIYFPPWLPLNLYDFGCWARQTIVPLTVVSAKRPVRPAPFSLDELHADPRRPNPPRPAAPLASWDGAFQRLDRALHLYRKVALRPLRRAALRSCARWIVERQENDGCWGGIQPPAVYSVIALHLLGYDLDHPVMRAGLESLDRFAVWREDGSRMIEACQSPVWDTCLAVIALADAGLAPDHPALVKSADWMLAEEIDRPGDWSVKRPRLAPGGWAFEFDNDNYPDIDDTAEVILALRRVDHPRPERIAAAVRRGVRWTLGMQSRNGAWGAFDVDNTSPLPNRLPFCDFGEVIDPPSADVTAHVVEMLAHEGGARDPRTRRAVGWLLAEQEPSGAWFGRWGTNYVYGTGSVVPALVAAGLPATHPAIRRAVRWLESVQNEDGGWGEDQRSYPDPEWIGHGASTASQTAWALLALLAAGERESKAVERGVGWLAATQDQDGSWDEPYFTGTGFPWDFSINYHLYRLVFPLTALGRYVSGEATGARPRRT

SEQ ID NO: 252 - protein, 680 aa, Streptomyces lividans
  source 1..680: note = strain: tk24; note = ZP_05522220.1 squalene-hopene cyclase [Streptomyces lividans TK24]
MTATTDGSTGASLRPLAASASDTDITIPAAAAGVPEAAARATRRATDFLLAKQDAEGWWKGDLETNVTMDAEDLLLRQFLGIQDEETTRAAALFIRGEQREDGTWATFYGGPGELSTTIEAYVALRLAGDSPEAPHMARAAEWIRSRGGIASARVFTRIWLALFGWWKWDDLPELPPELIYFPTWVPLNIYDFGCWARQTIVPLTIVSAKRPVRPAPFPLDELHTDPARPNPPRPLAPVASWDGAFQRIDKALHAYRKVAPRRLRRAAMNSAARWIIERQENDGCWGGIQPPAVYSVIALYLLGYDLEHPVMRAGLESLDRFAVWREDGARMIEACQSPVWDTCLATIALADAGVPEDHPQLVKASDWMLGEQIVRPGDWSVKRPGLPPGGWAFEFHNDNYPDIDDTAEVVLALRRVRHHDPERVEKAIGRGVRWNLGMQSKNGAWGAFDVDNTSAFPNRLPFCDFGEVIDPPSADVTAHVVEMLAVEGLAHDPRTRRGIQWLLDAQEADGSWFGRWGVNYVYGTGSVIPALTAAGLPTSHPAIRRAVRWLESVQNEDGGWGEDLRSYRYVREWSGRGASTASQTGWALMALLAAGERDSKAVERGVAWLAATQREDGSWDEPYFTGTGFPWDFSINYNLYRQVFPLTALGRYVHGEPFAKKSRAADAPAEAAPAEVKGS

SEQ ID NO: 253 - protein, 674 aa, Streptomyces peucetius
  source 1..674: note = subspecies: caesius; note = ACA52082.1 squalene-hopene cyclase [Streptomyces peucetius subsp. caesius ATCC 27952]
MTATTDGSSGPLRGGAATAGETTSTSAARTTEPGTDLREAAARAAERAVEHLLARQDAEGWWKGDLETNVTMDAEDLLLRQFLGIQDPATVGASARFIRGQQRDDGTWATFYGGPGELSTTVEAYVALRLAGDRPDDPHMQRAASWVRSRGGIAASRVFTRIWLALFGWWKWEDLPELPPELIFLPKWFPLNIYDFGCWARQTIVPLTVVSAKRPVRPAPFALDELHTDPALPNPGKRLAPAASWDGFFQRADKALHAYHKVAPRRLRRAAMNAAARWIIERQENDGCWGGIQPPAVYSVIALHLLGYDLEHPVMRAGLESLDRFAVHHEEEGLPVRMIEACQSPVWDTCLATIALADAGLPADHPALVKAADWMLSEQIVRPGDWSVRRPGLGPGGWAFEFHNDNYPDIDDTAEVVLALRRVKHPDPERVDAAVARGTRWNLGMQSRDGAWGAFDADNTSPFPNRLPFCDFGEVIDPPSADVTAHVVEILAHEGMAHDPRTRRGVRWLLAHQEANGAWFGRWGVNYVYGTGAVVPALTAAGLPGSHPAIRRAVAWLESVQNEDGGWGEDLRSYREEKSIGRGVSTASQTGWALLALLAAGERESKAVERGVAHLAQTQAPDGSWDEPYFTGTGFPWDFSINYHLYRQVFPLTALGRYVHGEKLPGRAGAREGR

SEQ ID NO: 254 - protein, 674 aa, Streptomyces pristinaespiralis
  source 1..674: note = strain: atcc 25486; note = EDY61772.1 squalene-hopene cyclase [Streptomyces pristinaespiralis ATCC 25486]
MTATTDGSSGPVRAGAATAGDTTTTTAARTTAPGTDVREAAGRAAERAVEHLLARQDAQGWWKGDLETNVTMDAEDLLLRQFLGIQDAATVEASARFIRGQQRDDGTWATFYGGPGELSTTIEAYVALRLAGDRPDDPHMQRAASWVRSRGGIAAARVFTRIWLALFGWWKWDDLPELPPELILLPKWVPLNIYDFGCWARQTIVPLTVVSAKRPVRPAPFALDELHTDPAMPNPQKRFAPAASWDGFFQRADKALHLYHKVAPRRLRRAAMNAAARWIIERQENDGCWGGIQPPAVYSVIALHLLGYDLEHPVMRAGLESLDRFAVHREEEGLPVRMIEACQSPVWDTCLATIALADAGLPADHPALVKAADWMLSEQIVRPGDWAVRRPGLGPGGWAFEFHNDNYPDIDDTAEVILALRRVKHPDPERVEAAVARGTRWNLGMQSLNGAWGAFDADNTSPFPNRLPFCDFGEVIDPPSADVTAHVVEMLAHEGMAEDPRTRRGVRWLLREQEANGAWFGRWGVNYVYGTGAVVPALIAAGLPASHPSVRRAVTWLESVQNEDGGWGEDLRSYREEQSIGRGASTASQTGWALLALLSAGERDGRAVERGVAWLARTQRPDGSWDEPYFTGTGFPWDFSINYHLYRQVFPLTALGRFLHGEKPVGRAAAREGG

SEQ ID NO: 255 - protein, 664 aa, Streptomyces scabiei
  source 1..664: note = strain: 87.22; note = CBG68454.1 putative squalene-hopene cyclase [Streptomyces scabiei 87.22]
MTATTDGSTGALPPRAPSASDTDHGTPVAAGVQEAALHAVGRATDFLLSRQDAQGWWKGDLETNVTMDAEDLLLRQFLGIRDDATTRAAALFIRGEQRPDGTWATFYGGPPDLSATVEAYVALRLAGDDPAAPHMAKASAWIRARGGIAAARVFTRIWLALFGWWKWDDLPEMPPEIVYFPTWMPLNIYDFGCWARQTIVPLTVVSAKRPVRPAPFPLDELHTDPGRPNPPRPLDRLGSWEGAFQRLDRALHGYHKVALKRLRRAAMNRAARWIVERQENDGCWGGIQPPAVYSVIALHLLGYDLGHPVMRAGLESLDRFAVWREDGARMIEACQSPVWDTCLATIALADAGLPPDHPQLVKAADWMLGEEIVRPGDWSVKRPQLPPGGWAFEFHNDNYPDIDDTAEVVLALRRVRHPDPERVERAVRRGVRWTLGMQSGNGAWAAFDADNTSPFPNRLPFCDFGEVIDPPSADVTAHVVEMLAAEGLSHDPRTRRGIEWLLAEQEPGGAWFGRWGVNYVYGTGSVVPALVTAGLPAAHPAIRRAVAWLETVQNDDGGWGEDLRSYPDPAEWGGKGASTASQTAWALLALLAAGERDGKATERGVAWLARTQREDGSWDEPYFTGTGFPWDFSINYHLYRQVFPLTALGRYVHGEPAVLKPGTR

SEQ ID NO: 256 - protein, 684 aa, Streptomyces sp.
  source 1..684: note = strain: mg1; note = EDX25760.1 squalene-hopene cyclase [Streptomyces sp. Mg1]
MRRRRSPRGPGAGPEADYGPARASAPDRLRGDAARGDAARRVQDATARAIRNLLGRQDPAGWWKGDLETNVTMDAEDLLLRQFLGIRDEAVTQAAALFIRREQREDGTWATFHGGPPELSATIEAYVALRLAGDAPDAPHMATASAWIRAHGGLAAARVFTRIWLALFGWWDWENLPELPPELVLLPPWVPLNIYDFGCWARQTIVPLTVVSAMRPVRPAPFALDELHTDARVPVPPRRMAPPTTWNGAFQWMDRALHVYRRFAPRRLREAAMASAGRWIIERQENDGCWGGIQPPAVYSVIALHLLGYDLGHPVMRAGLESLDRFAVWREDGSRMIEACQSPVWDTCLAAIALADAGVRPDHPALVKAADWMLGEEIVRTGDWAVRRPGLAPGGWAFEFHNDTYPDIDDTAEVVLALRRIRHPDPARVEAAIARGVSWNLGMQSRGGAWGAFDADNTSPFPNRLPFCDFGEVIDPPSADVTAHVVEMLAAEGRAADPRTRRGIAWLLAEQEPEGPWFGRWGTNYVYGTGSVVPALTAAGLSPGHPAIRRAVLWLESVQNPDGGWGEDQRSYQDRAWAGKGESTPSQTAWALMALLSAGERDAKTVERGIAYLVETQLADGGWDEPHFTGTGFPWDFSINYHLYRHVFPLTALGRYLYGEPFGHDGRHIGAHLGDRTGVPAEGV

SEQ ID NO: 257 - protein, 665 aa, Streptomyces sp.
  source 1..665: note = strain: spb074; note = EDY46371.1 squalene-hopene cyclase [Streptomyces sp. SPB074]
MHEGEAMTATTDGSTGAATPPATTASAPLHLSPEARETHEATARATRRAVDFLLARQSDEGWWKGDLATNVTMDAEDLLLRQFLGIRDEATTRAAALFIRGEQQEDGTWNTFYGGPGDLSATIEGYVALRLAGDSPEAPHMRKASAFVRAQGGVARARVFTRIWLALFGWWKWEDLPEMPPELMFFPKWAPLNIYDFGCWARQTIVPLTVVCAQRPVRPAPFALEELHTDPADPDPAQPAPPVVSWDNVFHKLDKLLHGYRRIAPRRVREAAMRAAATWIVERQENDGCWGGIQPPAVYSIMALNLLGYDLDHPVLRAGLASLDRFAVWREDGARMIEACQSPVWDTCLATVALADAGVPADHPQMIKAADWMLAEQIVRPGDWVVRRPDLPPGGWAFEFHNDNYPDIDDTAEVVLALRRVAHPDATRVDKAVRRAVDWNVGMQSKNGAWGAFDADNTSPFPNRLPFSDFGEVIDPPSADVTAHVVEMLAEEGLAHHPRTRRGIEWLLKNQEGNGSWFGRWGVNYVYGTGAVVPALVAAGLPASHPAIRRSVSWLGQVQNEDGGWGEDLRSYQDSAWHGRGHSTASQTAWALLALLAAGERETEQVRRGIAYLVETQTEDGTWDEPWFTGTGFPWDFTINYHLYRQVFPVTALGRYLNGTGPGEN

SEQ ID NO: 258 - protein, 636 aa, Streptomyces sp.
  source 1..636: note = strain: aa4; note = ZP_05479203.1 squalene-hopene cyclase [Streptomyces sp. AA4]
MTQTVPRTAASAPAARTAADTVAAAVQFLRREQDRAGWWKGELATNVTMDAEDLLLRHFLGILTPQIAEESARWIRSQQRADGTWANFPDGPADLSTTVEAWVALRLAGDPADAPWLATAAEWIREHGGIEATRVFTRIWLAMVGQWSWDDLPSLPPELIFLPSWFPLNVYDFACWARQTIVPLTIVGTLRPARKLPFDVAELRTGKRPPKPRAPWTWDGVFQNLDTALHAYAKLPLNPVRKLALKQAAEWILARQEADGSWGGIQPPWVYSILALHLLGYSLDHPALKAGIAGLDGFTIREKTDQGWVRRLEACQSPVWDTALAMTALLDAGVSPGDESLVRAAEWMLGEEIRVPGDWAVRRPSLKPGGFAFEFANDGYPDTDDTAEVVLALRRMGKPDHLRIREAVDRSVAWLEGMQSSDGGWGAFDADNTQVLTTRLPFCDFGAVIDPPSADVTAHVVEMLAAEGKADTRECRRGIRWLWDNQEADGSWFGRWGANYVYGTGAVVPALVAAGVPGTDPRIRRAVRWLAEHQNDDGGWGEDLRSYDDRSWAGRGDSTPSQTAWALLALLAAGERESTVVARGVEWLCERQRPDGGWDEDKHTGTGFPGDFYLSYHLYRVVFPLSALGRYVRGGS

SEQ ID NO: 259 - protein, 676 aa, Streptomyces sp.
  source 1..676: note = strain: c; note = ZP_05510126.1 squalene-hopene cyclase [Streptomyces sp. C]
MTATTDGGGAITGGADPRHDSTAAPAAAAAGPSGGGTGLPEGVREAVDRATAELLARQDPAGWWKGDLQTNVTMDAEDLLLRQFLGIRDEAVTRAAALFIRGEQQGDGTWATFHGGPPELSATIEAYVALRLAGDPPDAPHMTRASAWIRAHGGIAAARVFTRIWLALFGWWSWDRLPELPPELVFLPPWVPLNIYDFGCWARQTIVPLTVVSALRPVRSAPFALDELHTDARDPVPAKPLPPLASWDGAFQRMDKALHLYRRVAPRRLRKAAMAAAGRWIVERQENDGCWGGIQPPAVYSVIALHLLGYDLGHPVMRAGLESLDRFAVWREDGARMVEACQSPVWDTCLAAIALADAGLPPDHPALVRAADWMLGEEIRRPGDWAVRRPGLAPGGWAFEFHNDNYPDIDDTAEVVLALRRIRHPQPGGVEAAIARGVSWTLGMQSKNGAWGAFDADNTSPFPNRLPFCDFGEVIDPPSADVTAHVVEMLAAEGRAADPRARRGIAWLLAEQEPDGPWFGRWGTNYVYGTGSVVPALTAAGIAPSHPAVRRAVRWLESVQNEDGGWGEDQRSYRDRSWAGKGASTASQTAWALMALLSAGERDGDAVARGLAYLVETQRPDGTWDEPYFTGTGFPWDFSINYHLYRQVFPLTALGRYLHGEPFGPERRNVPPAGES

SEQ ID NO: 260 - protein, 684 aa, Streptomyces sp.; identical to SEQ ID NO: 256
  source 1..684: note = strain: mg1; note = ZP_05001249.1 squalene-hopene cyclase [Streptomyces sp. Mg1]

SEQ ID NO: 261 - protein, 665 aa, Streptomyces sp.; identical to SEQ ID NO: 257
  source 1..665: note = strain: spb074; note = ZP_04995097.1 squalene-hopene cyclase [Streptomyces sp. SPB074]

SEQ ID NO: 262 - protein, 606 aa, Streptomyces sp.
  source 1..606: note = strain: spb78; note = ZP_05490781.1 squalene-hopene cyclase - partial [Streptomyces sp. SPB78]
MDFLLDRQSDEGWWKGDLATNVTMDAEDLLLRQFLGIRDEATTQAAALFIRGEQQEDGTWNTFYGGPGDLSATIEGYVALRLAGDSPEAPHMRKASAFVRARGGVARARVFTRIWLALFGWWKWEDLPEMPPELMFFPKWAPLNIYDFGCWARQTIVPLTVVCAQRPVRPAPFALEELHTDPADPNPAQPAPPVASWDNVFHKLDKMLHGYRKVAPRRVREAAMRAAATWIVERQENDGCWGGIQPPAVYSIIALHLLGYDLDHPVLRAGLESLDRFAVWREDGARMIEACQSPVWDTCLATVALADAGVPADHPQMIRAADWMLAEQIVRPGDWVVRRPDLPPGGWAFEFHNDNYPDIDDTAEVVLALRRVAHPDATRVDKAVRRAVDWNAGMQSKNGAWGAFDADNTSPFPNRLPFSDFGEVIDPPSADVTAHVVEMLAEEGLAHHPRTRRGIEWLLENQEANGSWFGRWGVNYVYGTGAVVPALVAAGIPAAHPAIRRSVSWLGQVQNEDGGWGEDLRSYQDTAWHGRGHSTASQTAWALLALLAAGERDSEQVRRGIAYLVETQTEDGTWDEPWFTGTGFPWDFTINYHLYRQVFPVTALGR

SEQ ID NO: 263 - protein, 669 aa, Streptomyces sviceus
  source 1..669: note = strain: atcc 29083; note = EDY55942.1 squalene-hopene cyclase [Streptomyces sviceus ATCC 29083]
MHEGEAMTATTDGSTGALPPRAAAASETHLDTPVAAGIQEAAVRAVQRATEHLLARQDAEGWWKGDLETNVTMDAEDLLLRQFLGIRDESTTRAAAKFIRGEQREDGTWAGFYGGPGELSTTVEAYVALRLDGDAPDAPHMAKASAWIRAQGGIAAARVFTRIWLALFGWWKWEDLPELPPELIYFPKWAPLNIYDFGCWARQTIVPLTIVSAKRPVRPAPFPLDELHADPADPNPAKPLAPVASWDGAFQRLDKAMHQLRKVAPRRLRRAAMNSAARWIIERQENDGCWGGIQPPAVYSVIALHLLGYDLQHPVMRAGLESLDRFAIWREDGSRMIEACQSPVWDTCLATIALVDAGVPADHPQLVKAADWMLGEEIVRPGDWSVKRPQLPPGGWAFEFHNDNYPDIDDTAEVVLALRRVRHHDPDRVENAIGRGVRWNLGMQSKNGAWGAFDVDNTSPFPNRLPFCDFGEVIDPPSADVTAHVVEMLAVEGLSHDPRTRRGIEWLLAEQEPDGSWFGRWGVNYIYGTGSVVPALTAAGLPASHPAIRRAVAWLEKVQNDDGGWGEDLRSYKYVKEWSGRGASTASQTAWALMALLAAGERDSKAVERGVEWLASTQRADGSWDEPYFTGTGFPWDFSINYHLYRQVFPLTALGRYVHGEPFSRTEAL

SEQ ID NO: 264 - protein, 664 aa, Streptomyces viridochromogenes
  source 1..664: note = strain: dsm 40736; note = ZP_05535608.1 squalene-hopene cyclase [Streptomyces viridochromogenes DSM 40736]
MTATTDGSTGALPPRADAASEHDIETPEAAGVREAAVRAARRATDFLLSRQDAQGWWKGDLETNVTMDAEDLMLRQFLGVLDEKTAQAAALFIRGEQREDGTWASFYGGPGELSTTIEAYVALRLAGDAPDSPHLAKASAWIREQGGIAAARVFTRIWLALFGWWKWEDLPELPPELIWFPKWVPLNIYDFGCWARQTIVPLTIVSAKRPVRPAPFPLDELHTDPARPNPPRPLAPAFSWDGAFQRMDKGLHALRKVAPRGLRRAAMNAAARWIIERQENDGCWGGIQPPAVYSIIALHLLGYDLQHPVMREGLASLDRFAVWREDGARMVEACQSPVWDTCLAAIALVDAGLPADHPQLVKAADWMLGEEIVRPGDWSVRRPGLPPGGWAFEFHNDNYPDIDDTAEVILALRRITHHDPVRVDKAVGRGVRWTLGMQSKNGAWAAFDVDNTSPFPNRLPFCDFGEVIDPPSADVTAHVIEMLAVEGLAHDPRTRRGIEWLLAEQEPDGSWFGRWGVNYVYGTGSVVPALVAAGLPGAHPAIRRAVSWLESVQNDDGGWGEDLRSYKYVKEWSGRGASTASQTAWALMALLAAGERDSKAVERGVEWLAATQREDGSWDEPYFTGTGFPWDFSINYHLYRQVFPLTALGRYVHGEPFADRLKGS

SEQ ID NO: 265 - protein, 653 aa, Synechococcus sp.
  source 1..653: note = strain: pcc 7335; note = EDX84551.1 squalene-hopene cyclase [Synechococcus sp. PCC 7335]
MSISALQTDRLSQTLTQSVVAAQQHLLSIQNPEGYWWANLESNASITAEVVLLHKIWGTLDSQPLAKLENYLRAQQKTHGGWELYWNDGGELSTSVEAYMGLRLLGVPASDPALVKAKQFILHRGGVSKTRIFTKFHLALIGCYRWQGLPSLPAWVMQLESPFPFSIYELSSWARGSTVPLLIVFDKKPVYPLQPSPTLDELFTESAENVRWELEEKGDWSDAFLWLDKAFKLAESVDLVPFREESIRKAEKWVLERQEPSGDWGGIIPAMLNSMLALRALGYSVSDPVVRRGFQAIDNFMVESETECWAQPCISPVWDTGLAVRSLTDSGLSPNHPALVKAGEWLLDKQILSYGDWSVKNPQGQPGGWAFEFENSFYPDVDDTAVVAMALQDITLPNEPLKRRAIARAVRWIATMQCKTGGWAAFDINNDQDWLNDIPYGDLRAMIDPSTADITGRVLEMHGRFAADLDLANSYAADLSPYRLSRGLNYLIKEQELDGSWFGRWGVNYIYGTGQALSALALIAPERCRIQIERGIAWFVSVQNADGGWGETCESYKDKSLKGKGISTASQTAWALLGLLDVSFCLDPAAKIAVDRGIQYLVSTQSEGTWQEESFTGTGFPQHFYLRYRLYCHYFPLMALGRYQRVINSSAGI

SEQ ID NO: 266 - protein, 653 aa, Synechococcus sp.; identical to SEQ ID NO: 265
  source 1..653: note = strain: pcc 7335; note = ZP_05035816.1 squalene-hopene cyclase [Synechococcus sp. PCC 7335]

SEQ ID NO: 267 - protein, 647 aa, Synechocystis sp.
  source 1..647: note = strain: pcc 6803; note = BAA17978.1 squalene-hopene-cyclase [Synechocystis sp. PCC 6803]
MVIAASPSVPCPSTEQVRQAIAASRDFLLSEQYADGYWWSELESNVTITAEVVILHKIWGTAAQRPLEKAKNYLLQQQRDHGGWELYYGDGGELSTSVEAYTALRILGVPATDPALVKAKNFIVGRGGISKSRIFTKMHLALIGCYDWRGTPSIPPWVMLLPNNFFFNIYEMSSWARSSTVPLMIVCDQKPVYDIAQGLRVDELYAEGMENVQYKLPESGTIWDIFIGLDSLFKLQEQAKVVPFREQGLALAEKWILERQEVSGDWGGIIPAMLNSLLALKVLGYDVNDLYVQRGLAAIDNFAVETEDSYAIQACVSPVWDTAWVVRALAEADLGKDHPALVKAGQWLLDKQILTYGDWQIKNPHGEPGAWAFEFDNNFYPDIDDTCVVMMALQGITLPDEERKQGAINKALQWIATMQCKTGGWAAFDIDNDQDWLNQLPYGDLKAMIDPSTADITARVVEMLGACGLTMDSPRVERGLTYLLQEQEQDGSWFGRWGVNYLYGTSGALSALAIYDAQRFAPQIKTAIAWLLSCQNADGGWGETCESYKNKQLKGQGNSTASQTAWALIGLLDALKYLPSLGQDAKLTTAIEGGVAFLVQGQTPKGTWEEAEYTGTGFPCHFYIRYHYYRQYFPLIALARYSHLQAS

SEQ ID NO: 268 - protein, 632 aa, Trichodesmium erythraeum
  source 1..632: note = strain: ims101; note = ABG50159.1 squalene-hopene cyclase [Trichodesmium erythraeum IMS101]
MIEKNKVKQSILASQKHLLSLQETEGYWWGQLESNVTITAEIILLHKIWQTDKKIPLNKAKNYLISQQREHGGWELFYGDGGDLSTSIEAYMALRLLGVSRTDPIMIEAQNFIIKKGGISCSRIFTKLHLALIGCYSWQGIPSIPSSIMLLPEDFPFTIYEMSSWARSSTVPLLIVFDKKPIFSVNPTINLDELYAEGINNASFELPRKYDLTDLFLGLDKAFKFAENLNLMPLQQEGLKAAEKWILERQEVTGDWGGIIPAMLNSMLALKCLEYDVADPVVVRGLEAIDRFAIENEDSYRVQACVSPVWDTAWVIRSLVDSGISPSHPAMVKAGQWLLQQQILDYGDWVFKNKFGKPGGWAFEFMNRFYPDIDDTAVVVMALDVVELPDEDLKGKAIARGMEWIASMQCEAGGWAAFDVDNNQDWLNATPYGDLKAMIDPNTADVTGRVLEMVGCCGLAMDSWRVKRGIDFLVREQEEEGCWFGRWGVNYIYGTSGVILALAVMARESHRGYIERGASWLVGCQNSDGGWGESCWSYNDPSLKGKGKSTASQTAWALIGLLAAGEGTGNFARDAIDGGVGFLVSTQNDDGSWLEDEFTGTGFPGHFYIKYHFYSQYFPLMALGRYESLLSG

SEQ ID NO: 269 - protein, 640 aa, Verrucomicrobium spinosum
  source 1..640: note = strain: dsm 4136; note = ZP_02925563.1 probable squalene-hopene cyclase [Verrucomicrobium spinosum DSM 4136]
MATSDPSLAEAIQNTRAHLLSLRNARGHWEGHLSNSALSTATAIVALHLVDAPLHSARIAQGVRWLVLHQNKDGGWGDTTLSKSNLSTTLLCWSALSLCEPDRTEPIQHCEAWIKERTGSLEPEVICRAVVARYGKDKTFSVPILMLCAIGGRLGPEKEAWSRVLALPFELAAMPREWFGAIGLPVVSYALPALIAIGYARFYHAPPSLLNPLHALRKALWPRISPMLKLLQPSTGGYLEATPLTSFVTMALASAGEKFHPCVPEAVRFLEDSQRPDGSWPIDTNLATWGTTLSTKALTATSEGREALDIPALKSWLLEQQYQEIHPFTNAAPGGWAWTDLPGGVPDADDTSGALVALWHLCEDEAERQALAPAVAKGVQWLMDLQNRDGGIPTFCRGWGTLPFDRSTPEITAHALHAWGLWQVVLPEELQQEVSLRIPRAIAFIARPPSRGAPGFNHVPLWFGNEHAKEEENHVYGTAQIMNHLLSSGLNTPEIKVILETGHRNLLAWQQLDGGWSGSETGPASLEETAVSVAALALHTLHAGNRTRSSAEDAVAKGTQWLVQHTATGTTFPSAPIGLYFARLWYHEQLYPVIWTLGALHAVETLSAAALPLRARASAPPQHPGVVRTKPIHIAPPSDP

SEQ ID NO: 270 - protein, 649 aa, Acidiphilium cryptum
  source 1..649: note = strain: jf-5; note = ABQ30890.1 squalene-hopene cyclase [Acidiphilium cryptum JF-5]
MFDTISFDFDALDQAISRAHARLSAEQRADGHYVYELEADATIPAEYVLLEHFLDRIDPELEARIGVFLRGIQGNSPQNPGGWPLFHDGAMDISASVKAYFALKAIGDDPDAPHMRRAREAILARGGAARTNVFTRIQLALFGAVPWRACPVMPVEIMLLPDWFPITIWKISYWSRTVIAPLLVLLTERPIARNPRNVRIDELFVTPPDQVTDYIRGPYRSNWGYLFKAIDSALRPLERHFPARSRKRAIQAAIDFITPRLNGEDGLGAIYPAMANTVMMYHTLGYSPDHPDYATAWASVRKLVTDASYRFEGASYVQPCLSPVWDTSLAAHALAEAGSPGDAQLAAACDWLIPRQILDVKGDWAYRKPDAPPGGWAFQYNNAHYPDVDDTAVVGMILDRNGDPAHREAVERARQWILGMQSRSGGWGAFDSDNEFHYLNHIPFADHGALLDPPTADVTARCISFLAQLGHAEDRPAIERGVAYLRREQEQDGSWFGRWGTNYIYGTWSSLCALNAAGVAQDDPMMVRAVEWLLARQRPDGGWGEDCETYAHAKPGEYHESLPSQTAWALLGLMAAGQAEHEAVARGIAWLQSVQEDDGSWTEQPYNAVGFPRVFYLRYHGYPRFFPLLAMARYRNLARGNSRQVQFGF

SEQ ID NO: 271 - protein, 699 aa, Acetobacter fabarum
  source 1..699: note = PAK78064.1 squalene--hopene cyclase [Acetobacter fabarum]
MNTISHPSKVKAAVSATVPPTPSCVTPTPFTGMGNSLAHTVAAACDWLIGEQKADGHWVGPVASNASMEAEWCLALWYLGLEDHPLRPRLGKALLHMQREDGSWGTYWGAGNGDINATVEAYAALRSLGYAADTPELSKACAWIMRMGGLRNVRVFTRYWLALIGEWPWEQTPNLPPEVIWFPNKFVFSIYNFAQWARATLVPLAILSARRPSRPLRPQDRLDALFPQGRENFDYVLPKKEGVDLWSSFFRTTDKGLHWLQSRFLKRNTVREAAIRHMLEWIIRHQDADGGWGGIQPPWVYGLMALHGEDYQFHHPVMAKALAALDDPGWRRDQGDASWVQATNSPVWDTMLALMALHDANAEERYTPQMDKALDWLLARQVRVKGDWSIKLPDVEPGGWAFEYANDRYPDTDDTAVALIALSSCRNREEWKEKGVEDAITRGVNWLIAMQSSCGGWGAFDKDNNRSLLSKIPFCDFGEALDPPSVDVTAHVLEAFGLLGVPRQTPALQRGLAYIRAEQEASGAWFGRWGVNYLYGTGAVLPALAAIGEDMTQPYITRACDWLIAHQQEDGGWGESCASYMDVSSIGWGTTTPSQTAWALMGLIAANREQDHPAIARGCRYLIDRQETDGSWTEEEFTGTGFPGYGVGQTIKLDDPAVAKRLQQGAELSRAFMLRYDLYRQFFPLMALSRAARIMPVGQ

SEQ ID NO: 272 - protein, 693 aa, Acetobacter orleanensis
  source 1..693: note = strain: jcm 7639; note = GAN69910.1 squalene--hopene cyclase [Acetobacter orleanensis JCM 7639]
MTTPLFKGMGNSLTHTVSSACEWLISQQNPDGHWVGPVGSNASMEAEWCLALWFLGLEDHPLRPRLGNALLQTQREDGSWDVYLGAGNGDINATVEAYAALRSLGYPENTPALQKAATWIKQKGGLKNIRVFTRYWLALIGEWPWEKTPNLPPEIIWFPNKFVFSIYNFAQWARATLVPLAILSARRPSRPLRPQDRLNALFPEGRGNFDYTLPKKEGVDLWSDFFRTTDKGLHWLQSKFLKRNTMREAAIRHMLEWIIRHQDADGGWGGIQPPWVYGLMALHGEGYQFHHPVMAKGLDALNDPGWRHDKGNASWIQATNSPVWDTMLAIMALHDAKAEDRFTPQVDKALGWLLDRQVRVKGDWSIKLPDVEPGGWAFEYANDFYPDTDDTAVALIALASCRHRPEWQERGVEDAIARAVRWLVAMQSSCGGWGAFDKDNNKALLSKIPFCDFGEALDPPSVDVTAHILEAFGLLGLPRDLPCIKHALDYVRAEQDPQGPWFGRWGVNYVYGTGAVLPALAAIGEDMTQPYITKACDWLVAHQQEDGGWGESCASYMDASTIGRGKTTPSQTAWALMGLIAAARPQDYPAIEKGCRYLIDRQEPDGSWTEEDYTGTGFPGYGVGQTIRLDDPALSKRLQQGAELSRAFMLRYDLYRQFFPIMALSRASRLISPETATEQAVEAAAKNLEKIIA

SEQ ID NO: 273 - protein, 720 aa, Acetobacter pasteurianus; identical to SEQ ID NO: 24
  source 1..720: note = WP_012812952.1 squalene--hopene cyclase [Acetobacter pasteurianus]

SEQ ID NO: 274 - protein, 633 aa, Acidothermus cellulolyticus; identical to SEQ ID NO: 58
  source 1..633: note = WP_011720532.1 squalene--hopene cyclase [Acidothermus cellulolyticus]

SEQ ID NO: 275 - protein, 631 aa, Alicyclobacillus acidocaldarius [sequence omitted]

SEQ ID NO: 276 - protein, 625 aa, Priestia megaterium
  source 1..625: note = WP_013083001.1 squalene--hopene cyclase [Priestia megaterium]
MIILLKEVQLEIQRRIAYLRPTQKNDGSFRYCFETGVMPDAFLIMLLRTFDLDKEVLIKQLTERIVSLQNEDGLWTLFDDEEHNLSATIQAYTALLYSGYYQKNDRILRKAERYIIDSGGISRAHFLTRWMLSVNGLYEWPKLFYLPLSLLLVPTYVPLNFYELSTYARIHFVPMMVAGNKKFSLTSRHTPSLSHLDVREQKQESEETTQESRASIFLVDHLKQLASLPSYIHKLGYQAAERYMLERIEKDGTLYSYATSTFFMIYGLLALGYKKDSFVIQKAIDGICSLLSTCSGHVHVENSTSTVWDTALLSYALQEAGVPQQDPMIKGTTRYLKKRQHTKLGDWQFHNPNTAPGGWGFSDINTNNPDLDDTSAAIRALSRRAQTDTDYLESWQRGINWLLSMQNKDGGFAAFEKNTDSILFTYLPLENAKDAATDPATADLTGRVLECLGNFAGMNKSHPSIKAAVKWLFDHQLDNGSWYGRWGVCYIYGTWAAITGLRAVGVSASDPRIIKAINWLKSIQQEDGGFGESCYSASLKKYVPLSFSTPSQTAWALDALMTICPLKDRSVEKGIKFLLNPNLTEQQTHYPTGIGLPGQFYIQYHSYNDIFPLLALAHYAKKHSS

SEQ ID NO: 277 - protein, 632 aa, Bacillus; identical to SEQ ID NO: 104
  source 1..632: note = WP_004399534.1 MULTISPECIES: sporulenol synthase [Bacillus]

SEQ ID NO: 278 - protein, 617 aa, Bacillus thuringiensis
  source 1..617: note = AJI35613.1 squalene-hopene cyclase [Bacillus thuringiensis]
MLLYEKAHEEIVRRATALQTMQWQDGTWRFCFEGAPLTDCHMIFLLKLLGRDKEIEPFVERVASLQTNEGTWKLYEDEVGGNLSATIQSYAALLASKKYTKEDANMKRAENFIQERGGVARAHFMTKFLLAIHGEYEYPSLFHLPTPIMFLQNDSPFSIFELSSSARIHLIPMMLCLNKRFRVGKKLLPNLNHIAGGGGEWFREDRSPVFQTLLSDVKQIISYPLSLHHKGYEEIERFMKERIDENGTLYSYATASFYMIYALLALGHSLQSSMIQKAIAGITSYIWKMERGNHLQNSPSTVWDTALLSYALQEAQVSKDNKMIQNATAYLLKKQHTKKADWSVHAPALTPGGWGFSDVNTTIPDIDDTTAVLRALARSRGNKNIDNAWKKGGNWIKGLQNNDGGWGAFEKGVTSKLLAKLPIENASDMITDPSTPDITGRVLEFFGTYAQNELPEKQIQRAINWLMNVQEENGSWYGKWGICYIYGTWAVMTGLRSLGIPSSNPSLKRAASWLEHIQHEDGGWGESCHSSVEKRFVTLPFSTPSQTAWALDALISYYDTETPAIRKGVSYLLSNPYVNERYPTGTGLPGAFYIRYHSYAHIYPLLTLAHYLKKYRK

SEQ ID NO: 279 - protein, 618 aa, Brevibacillus thermoruber
  source 1..618: note = WP_029099368.1 squalene--hopene cyclase [Brevibacillus thermoruber]
MNSELERLTAVLQREQQADGSWRYCFESGPLTDAYAIILLRTLNIPNEPLISGLAKRIASRQAPDGTWKLYSDERDGNLSTTIESYFALLAAGAAAPSDERLQAARRFIRAKGGLAQANLGTRVMLALTGQHPWPPFPIPAEFMLVPPFFPLHLFDLVGFARVHLVPIMVAAARTFAVRSRQMPDLSDLFRGLPSAGPQQLDLIWFHELIDSGIRSLPSFLRPARELGLREAERFLLDRLEPDGTLYSFFTATFLMIFALLALGYRPDHPVIIRAVRGAERLVCPVGDVLHMQNSTSTIWDTALLSHALQTAGMPVSHPVIQQATRYLLSRQHNRYGDWARRSPGVPPGGWGFSDINTINPDVDDTTAALRALHRAGSGDPAIRQAWDRGLRWLLSMQNSDGGWPAFERNTANPLVKLLPAGGAEAAFTDPSTADLTGRTLEFLGNHAGMTLQHPAVRRGVDWLLRHQETNGSWHGRWGISYLYGTWAALSGLIAAGVSPDHPAIQKGVSWLRSVQNRDGGWGESCQSDVLKRYVPLGASTPSQTAWAVDALTAVSRRTGPELEAGVRFLLAAGKRRDWTSSYPTGAALPGGFYIHYHSYRYIWPLLTLAQYRNKFQP

SEQ ID NO: 280 - protein, 659 aa, Chloracidobacterium thermophilum
  source 1..659: note = WP_014100779.1 squalene--hopene cyclase [Chloracidobacterium thermophilum]
MPGFAPRFVQPVVESPLPPAFRSARPAPATAAAVEAAIRKAQAYLLSKQYPEGYWWAELEANVTLTAEYVFLHKVLGTDGERTRQFEKIRTYLRRQQREHGGWELYYGDGGELSTSIEAYFALKLLGDSPDLPHMARARQFILARGGITKARVFTKIHLALFGAFPWEGCPTLPPWIMLLPDWFPFTIYELASWARSSTVPLLLVSDRKPVVRVPGGDADELYAEGRAQADLSLPNPAGLLSLGGVFIGFDWMLKLMERFDLSPRRAEALARAEQWTLEHQDDSGDWGGIIPAMLNSLLGLHCRGYAPTHPVMQKGIAAVERFCIETEDEFHTQPCVSPVWDTGLTILALLDSGLPNDHPALVRAGEWLLSKQIFRDGDWRFKNRTGPAGGWAFEFWNDFFPDVDDTAVVTMALHRLKLPDEAEKQRRLKLAIEWTLSMQSKNGGWGAFDVDNTLEILNDIPYGDLKAMIDPPTADLTGHILEMLGVTGYAAPREKVERAIAFIKSKQEPEGCWWGRWGVNYIYGTHMVICGLVALGLDPREAFIMRGTQWLNSCQNEDGGWGETCASYGDRTLMGVGKSTPSQTAWALLGLMAGGEGKSDCARRGIEYLVTHQNDDGSWTEAEFTGTGFPNHFYMNYHFYRNYFPLMALGRYRAFART

SEQ ID NO: 281 - protein, 636 aa, Cohnella thermotolerans
  source 1..636: note = WP_027091823.1 squalene--hopene cyclase [Cohnella thermotolerans]
MNLSGQVDQAVGRLSESLSRMQSDDGSWRFCYENAVLTDAYMIIALRTLEIADEPLIRQLRDRLLATQYADGAWRAYPDEREGNLSATVECYYALLYSGYSRDADPPLVKARAFILAKGGIQQIGGLLTKVMLASTGQYPWPRSLKIPLEFLLLPSQFPLSVFDFSGYARVHMIPALLLADRRFSLRTKTSPDLSELAGDRSREPPSWFDPALGRGHPRELQSLLEQIGKGIERLNGIPSQLHEEAVRRAERYMLERIEADGTLYSYATSTFLMILALLAIGYDKRHAVIRNAVQGLRAMVCRGAQPIFLQNAPSTVWDTALLSSALQEAGADANSPMIRRANAYLLAKQHRKPGDWLVHNPSAVPGGWGFSDTNTINPDVDDTTAALRAIKRQAGADPAYREAWNRGLHWLLSMQNDDGGWPAFEKNTDRQILVLLPLREAKSSAIDPSTSDLTGRTLEFLGNYAGLGMGHAFIRRGTDWLIGHQEKDGSWYGRWGVCYIYGTWAALTGLAAAGIRADHPAVRAGAQWLTDIQQADGGWGESCDSDRQMRYIPLEESTPSQTAWALDALIAVHEAPTPTIDRGIRRLIGLLQEESRFAAYPTGAGLPGIFYSHYYSYRYIWPLFALAHYKSKYSS

SEQ ID NO: 282 - protein, 617 aa, Geobacillus thermodenitrificans
  source 1..617: note = WP_029761705.1 squalene--hopene cyclase [Geobacillus thermodenitrificans]
MAGERSALITALKRSQAADGSWRFPFETGISTDAYMIILLRTLDINDEPLIQALVERIESRQEANGAWKLFADEGDGNVTATVEAYYALLYSGYRQPTDRHMQKAKRRILDMGGLDRIHLFTKVMLALTGQYPWPGRFPLPLEFFLLPPSFPLNMYDLSVYGRANMIPLLIAADSRYSRKTDKSPDLSDLFASRGDWGMPESRSLLTYVKRSLIGLPAQLHQAAKQRAVRYLFEHIEPDGTLYSYFSSTFLFIFALLALGYRNDDPRIRQAVRGLRSLRTTIDGHVHLQYTTASVWNTALASYTLQEAGVPMTDRAIEKANRYLLSRQHVRYGDWAVHNPYSTPGGWGFSDVNTMNPDVDDTTAALRAIRQAAAKETAFRHAWDRANQWLFSMQNDDGGFAAFEKNVSSRFWRYLPIEGAEFLLMDPSTADLTGRTLEYFGTFAGLTKDQRAVSRAVDWLLSHQERNGSWYGRWGICYIYGTWAAITGLTAVGVPAHHPALQKAVRWLLSIQNDDGGWGESCKSDGAKTYVPLGDSTPVHTAWALDALVAAAERPTPEMKAGFRALFRLLHHPDWTASYPVGQGMAGAFYIHYHSYRYIFPLLALAHYEQKFGPLDD

SEQ ID NO: 283 - protein, 618 aa, Geobacillus
  source 1..618: note = WP_031409036.1 MULTISPECIES: squalene--hopene cyclase [Geobacillus]
MVADERSVLIDALKQSQAADGSWRFPFETGISTDAYMIILLRTLEINDEPLIQALVERIESRQEANGAWKLFADEGDGNVTATTEAYYALLYSGYRKKTDPHMQKAKMRILEMGGLENVHLFTKVMLALTGQHPWPRRFPLPLAFFLLPPSFPLNMYDLSVYGRANMIPLLVAAERRYSRKTAKSPDLSDLAASRGGWRLPENRALWSYIKRALTGFPDELHDAAKQRAVRYMFGHIEPDGTLYSYFSSTFLFIFALLALGYPKDDLHIARAVRGLRSLRTEIDGHTHMQYTTASVWNTALASYALQEAGVPSTDRTIEKANRYLLSRQHIRYGDWAVHNPHSLPGGWGFSDVNTMNPDVDDTTAALRAIRRAAAKETAFRHAWDRANRWLFSMQNNDGGFAAFEKNVGKRFWRYLLIEGAEFLLMDPSTADLTGRTLEYFGTFAGLTKDHPAVARAVDWLLDHQEADGSWYGRWGICYVYGTWAAVTGLSAVGVSADHPAMQKAVHWLLSIQNADGGWGESCKSDGAKTYVPLGASTPVHTAWALDALIAAAERPTPEMKAGVRALVRMLHHPDWTASYPVGQGMAGAFYIHYHSYRYIFPLLALAHYEQKFCLIAN

SEQ ID NO: 284 - protein, 706 aa, Novacetimonas hansenii
  source 1..706: note = CUW48332.1 Squalene--hopene cyclase [Novacetimonas hansenii]
MDTTGHTPVTTAPAAPDATGTQTQTAGAPFAGMGNSMTHTISAACDWLIQQQKPDGHWVGSVGSNASMEAEWCIALWFLGLENHPLRPRLGNALLEMQREDGSWGVYHGAGNGDINATVEAYAALRSLGYPADTPALARAATWIARKGGLRNIRVFTRYWLALIGEWPWEKTPNLPPEIIWFPNKFVFSIYNFAQWARATLVPLAILSARRPSRPLRPQDRLDALFPEGRANFDYTLPKKEGRDLWATFFRTTDRGLHWLQSNVLRRSTMRGAAIRHMLEWIIRHQDADGGWGGIQPPWVYGLMALHGEDYQLHHPVMARSLGALDDPGWRHDRGNASWIQATNSPVWDTMLALMALHDAGGEDRFTPEMDRALDWLLARQVRVRGDWSIKLPDVEPGGWAFEYANDRYPDTDDTAVALIALAPCRNRPEWKEKGVDAAIDRAVRWLVAMQSECGGWGAFDKDNNRSLLSKIPFCDFGEALDPPSVDVTAHILEAFGLLGLPRDMPAIQRALAYVRAEQDPAGPWFGRWGVNYVYGTGAVLPALAAIGEDMTQPYIARACDWLVAHQQEDGGWGESCASYMEIASVGRGTTTPSQTAWAVMGLVAANRAQDYPAIARGCRYLIERQQPDGSWHEAEFTGTGFPGYGVGQTIKLDDPMLSQRLSQGAELSRAFMLRYDLYRQLFPIMALSRAARLMPVGGAKQQGTV

SEQ ID NO: 285 - protein, 680 aa, Gluconobacter frateurii
  source 1..680: note = strain: nbrc 103465; note = GAD08844.1 squalene--hopene cyclase [Gluconobacter frateurii NBRC 103465]
MGVWRMSAPILKGMSNSLAHTVSCACDWLIGQQKADGHWVGSVESNASMEAEWCLALWFLGLEDHPLRPRLGNALLEMQRDDGAWGVYLGAQSGDINATVEAYAALRSLGYSANSPVLLKAGAWISEKGGLKNIRVFTRYWLALIGEWPWEKTPNLPPEIIWFPNNFVFSIYNFAQWARATLAPLAILSARRPSRPLRPQDRLDALFPEGREKFDYTLPKKDRVDLWSSFFRTTDKGLHWLQSRFLKRNTVREAAIRHMLEWIIRHQDADGGWGGIQPPWVYGLMALHGEGYPFHHPVMAKALAALDDPGWRYDRGEASWIQATNSPVWDTMLALMALHDANAQERFTPEMDKALGWLLERQVRVKGDWSIKLPDVEPGGWSFEYANDRYPDTDDTAVALIALSFCRHREEWKQKGVDKAIDRAVNWLIAMQSSCGGWGAFDKDNNKSLLSKIPFCDFGEALDPPSVDVTAHILEAFGLLGLSRDLPVVQKALAYVRLEQDPQGPWFGRWGVNYLYGTGAVLPALAAIGEDMTQPYILKACEWLISCQQDDGGWGESCASYMDISSIGRGSTTASQTAWALMGLIAVGRPQDHEAIAKGCRFLIDRQEADGSWTEEEFTGTGFPGYGVGQTIKLDDPALSKRLMQGAELSRAFMLRYDMYRQYFPIMALARASRLLTQDI

SEQ ID NO: 286 - protein, 700 aa, Komagataeibacter nataicola
  source 1..700: note = AQU88860.1 squalene--hopene cyclase [Komagataeibacter nataicola]
MNSESRLSRKQAGAPGPDKIEARPDSTPAAAFRGIDNSLTHTLSSACKWLMEQQKPDGHWVGSVASNASMEAEWCLALWFLGLEDHPLRPRLGKALLEMQREDGSWGIYYGAGNGDINATVESYAALRSLGYAADDPALSRAATWIASKGGLRNVRVFTRYWLALIGEWPWEKTPNLPPEIIWFPNKFVFSIYNFAQWARATLVPLAILSARRSSRPLRPQDRLDALFPGGRENFDYELPPRDGQDLWATFFRKTDRALHWLQTKFLKRNTMREAAIRHMLEWIIRHQDADGGWGGIQPPWVYGLMALHGEDYQFHHPVMAKGLAALDDPGWRYDRGDASWVQATNSPVWDTMLALMALHDADAETDFTPEMDKALGWLLERQVRVKGDWSVKLPDLEPGGWAFEYANDRYPDTDDTAVALIALAACRDREEWKGRGVEAAITRGVNWLVGMQSTCGGWGAFDKDNNRALLSKIPFCDFGEALDPPSVDVTAHVLEAFGVLGLPRDMPALQRGLAYIRAEQEADGPWFGRWGVNYLYGTGAVLPALAAIGEDMTQPYIARACDWLVAHQQENGGWGESCASYMEIASIGRGPTTPSQTAWALMGLIAANRKQDHEAIVRGCRYLIDQQQADGSWEEKEFTGTGFPGYGVGQTIKLDDPALTSRLQQGAELSRAFMLRYDLYRQFFPIMALSRAVRVLKGS

SEQ ID NO: 287 - protein, 714 aa, Komagataeibacter xylinus
  source 1..714: note = strain: e25; note = AHI26287.1 squalene--hopene cyclase [Komagataeibacter xylinus E25]
MTRESRPLTKTAASSGNITSGNTFPAVGTQSVNGGGKSTGSASALRTMDNSLSHAISSACDWLVGQQKPDGHWVGPVASNASMEAEWCLALWFLGLDDHPLRPRLGKALLEMQREDGSWGTYYGAGNGDINATVESYAALRSLGYPADDPAISRAATWIASKGGLKNIRVFTRYWLALIGEWPWEKTPNLPPEVIWFPNNFVFSIYNFAQWARATLVPLAILSARRPSRPLRPQDRLDALFPGGRANFDYELPARGDRDLWDRFFRATDRGLHWLQSRFLKRNTLREAAIRHMLEWIIRHQDADGGWGGIQPPWVYGLMALHGEDYQFHHPVMAKALSALNDPGWRHDKGDASWIQATNSPVWDTMLAIMALHDADGETQFSPQMEKALGWLLDRQVRVKGDWSIKLPDVEPGGWAFEYANDRYPDTDDTAVALIALSSCRNREEWKKRGVEEAISRGVNWLIGMQSECGGWGAFDKDNNRSILSKIPFCDFGEALDPPSVDVTAHVLEAFGILGLPRHMPTIQRALAYIRAEQEPDGPWFGRWGVNYLYGTGAVLPALAAIGEDMTQPYITKACDWLVAHQQENGGWGESCASYMELSMVGRGVTTPSQTAWALMGLIAANRPQDYGAIARGCRYLIDLQQADGSWHEKEFTGTGFPGYGVGQTIKLDDPALSKRLQQGAELSRAFMLRYDLYRQFFPIMALSRASRLMKLEK

SEQ ID NO: 288 - protein, 689 aa, Methylacidiphilum fumariolicum
  source 1..689: note = WP_009061034.1 squalene--hopene cyclase [Methylacidiphilum fumariolicum]
MHSGRLFLKKENEVGDNKKLHSVPLSLVEETLNFPQKVEKTIKKAQRYLLSIQKEDGHWVGELFVDVTLACDCIHLMHWRGKIDYKKQKRLVKHILDRQLPDGGWNIYPGGPSEVNATVKAYFALKLAGFSPDEPLMAKARSTILRLGGIPKCMTYTKLGLALLGVYPWDRLPVIPPEIILFPNWFPFNLYEISAWSRTMLVPLSIIHHFKPTRILPEKLQLHELFPYGTERGKFSWLKKGAKYLSKEGLFLACDKFLQYWDKTSLKPFRKMAIEKAEKWILERIAAGSDGLGAIFPAMHYAIMALIALGYTEDNPILKKAITDFESLEVDDQKNDDLRIQPCLSPLWDTAIGLVALAESGYERNAPQLKKAAHWIINREIRIKGDWYVRNPHPEASGWAFEYNNMYYPDVDDTLMVLLALRLIDIDDKIKKEEVMQRALRWVISFQCENGGWAAFDKNVYKKWLEDIPFADHNAILDPPCSDITARALELFGKMGIRKNEKFVQKAIRYLKETQESDGSWMGRWGVNYIYGTWQALRGLQAIGEDMNQEWILRARDWLESCQNEDGGWGETPASYDNPQLKGKGPSTASQTAWAISGIMACGDIFRPSITRGIKYLCERQLSDGSWAEEFLTGTGFPGVFYLKYDMYRNAWPLLVIGEYYRQYQQAKERATYWVDATLGCMEKRLSAV

SEQ ID NO: 289 - protein, 654 aa, Methylococcus capsulatus
  source 1..654: note = WP_010960137.1 squalene--hopene cyclase [Methylococcus capsulatus]
MLREATAISNLEPPLTASYVESPLDAAIRQAKDRLLSLQHLEGYWVFELEADCTIPAEYILMMHFMDEIDAALQAKIANYLRSHQSADGSYPLFRGGAGDISCTVKVYYALKLAGDSIDAPHMKKAREWILAQGGAARSNVFTRIMLAMFEQIPWRGIPFIPVEIMLLPKWFPFHLDKVSYWSRTVMVPLFILCSHKVTARNPSRIHVRELFTVDPQKERHYFDHVKTPLGKAILALERFGRMLEPLIPKAVRKKATQKAFDWFTARLNGVDGLGAIFPAMVNAYEALDFLGVPPDDERRRLARESIDRLLVFQGDSVYCQPCVSPIWDTALTSLTLQEVARHTADLRLDAALSKGLKWLASKQIDKDAPGDWRVNRAGLEGGGWAFQFGNDYYPDVDDSAVVAHALLGSEDPSFDDNLRRAANWIAGMQSRNGGFGAFDADNTYYYLNSIPFADHGALLDPPTADVSARCAMFLARWVNRQPELRPVLERTIDYLRREQEADGSWFGRWGTNYIYGTWSVLLAYEAAGVPNDDPSVRRAVAWLKSIQREDGGWGEDNFSYHDPSYRGRFHTSTAFQTGFALIALMAAGEAGSPEVQAGVDYLLRQQRPDGFWNDECFTAPGFPRVFYLKYHGYDKFFPLWALARYRNERYALA

SEQ ID NO: 290 - protein, 617 aa, Sphaerobacter thermophilus; identical to SEQ ID NO: 243
  source 1..617: note = WP_012872483.1 squalene--hopene cyclase [Sphaerobacter thermophilus]

SEQ ID NO: 291 - protein, 710 aa, Syntrophobacter fumaroxidans; identical to SEQ ID NO: 33
  source 1..710: note = WP_011698842.1 squalene--hopene cyclase [Syntrophobacter fumaroxidans]

SEQ ID NO: 292 - protein, 671 aa, Teredinibacter turnerae; identical to SEQ ID NO: 34
  source 1..671: note = WP_015819476.1 squalene--hopene cyclase [Teredinibacter turnerae]

SEQ ID NO: 293 - protein, 630 aa, Thermoactinomyces sp.
  source 1..630: note = strain: gus2-1; note = KFZ40906.1 hypothetical protein JS81_04530 [Thermoactinomyces sp. Gus2-1]
MQSQWILHVQAMIHRLKKELIQKQNPDGTWPFCFENGIGTDAYYVLLHQVLKRPDPGTLAPVLERILDKQTGDGTWKAFPDEKEGNVSATLDASLALLYSGVKTPDDPSLKRARDFLLSRGMETKAGSLTQVVLALLGHRSWSRITKLPVEFFLLPAASPVNFFDFVGYARVHIAPVMLASDQDFYIHLKGYREVEDWLPSSFRTYMERMHPDYFWTKEDLLPAVETTAYSTFFKKNVHQRALYWGENFLLSRIEEDGTLYSYMTSTFLMIFALLSLDYPPDHPLIQKAMEGLDRMIFPLQEGAHLQEATSTVWDTSLVMTALQNAGLSPGHGVIQKGRNYLLLKQHTSCGDWCLKNRYAIPGGWGFSDTNTINPDVDDTVQCLHAIAPAVREGWAQDEWKRGLQWLLSMQNRDGGWPAFERNTNKMWLKLLPARNEKRVWGDPSTADLTGRVLHFLGSELGWTIDRPEVRRAWSWLYHHQNSDGSWFGRWGVSYIYGTWAALKGLAAVGVPETHVSVQKGIRFLLSKQRPDGGWGESCYSDAEDRFVPLSFSTPVQTAWALDALIAYHDHPTPAIEKGMACLLEMMEKRGEEWSYPAGAGLSGQFYVYYHSYPYVWSLMAMTHYLQKYS

SEQ ID NO: 294 - protein, 725 aa, Zymomonas mobilis; identical to SEQ ID NO: 13
  source 1..725: note = WP_011241313.1 squalene--hopene cyclase [Zymomonas mobilis]

SEQ ID NO: 295 - protein, 631 aa, Alicyclobacillus acidocaldarius [sequence omitted]

SEQ ID NO: 296 - protein, 653 aa, Pseudomonas mendocina
  source 1..653: note = PmeSHC
MHNQPFPRDNLSIAVGQSSGATSNADLTEAVDRATDALLARQLPEGHWHYPLESDVATTAHYLLAARYLGEAIECEVEVKIAGYLRARQLPEGGWPLDADGPINVSSSVLAYFALKLLGDAPDAPHMRRACTAIRAHGGAESSNVFARFMLALFGLVPWRALPTMPVILLLAPRWFPFHLAKMASWARLVLVPLLVISNQRPLAKNLQNTGLEELFIHLPSRLGLPPRAPHQQPARFACLWAVDRLLGLFDKAWPKRLKQRAIDTALTFVGERLNGESGLGGLHVATMLASLMYETVGFPFEHPMRVMARQASNKLLVIEEEQAYCQPSESPLWDTAWALHALLETQTLRGCSAALQGAQWLRTLQILEVHGDWAQQAPDLPPGGWAFQYRNERYPDVDDSAVVAAALHRASRLNADKAGLEATRRACRWVAGLQSRNGGWGAYDRDNTAEYLNDLPFADHAAMTDAPTADVTSRCLSLLAPFQQDAEEARVVRDAVDCLWAMQESDGSWLGHWGVTYIYGTWSALCALDTAGVDHQDPRMQRAARWLVSIQNQDGGWGESAGAHEVSDVGHYRAASTASQTAWAMLGLMAAGAVGHPAVSRGADYLIATQRDQGGWAESGFTGAAVQKVMYLRYHGYSEYFPLWALARYRNL

SEQ ID NO: 297 - DNA, 1896 bp, synthetic construct
  source 1..1896: mol_type = other DNA; note = nucleotide sequence of SHC/HAC enzyme variant #66
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgcacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgtgggctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacgacctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtacagccacgtgtttccgacgctcgcgctcggccgctacaagcaagccatcgagcgcaggtga

SEQ ID NO: 298 - DNA, 1896 bp, synthetic construct
  source 1..1896: mol_type = other DNA; note = nucleotide sequence of SHC/HAC enzyme variant #110B8
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgcacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgtgggctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaacctcaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacgacctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtaccgccacgtgtttccgacgctcgcgctcggccgctacaagcaagccatcgagcgcaggtga

SEQ ID NO: 299 - DNA, 1896 bp, synthetic construct
  source 1..1896: mol_type = other DNA; note = nucleotide sequence of SHC/HAC variant #90C7
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacgcgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgtgggctcgggcgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacatgacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacggcctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtacagccacgtgtttccgacgctcgcgctcggccgctacaagcaagccatcgagcgcaggtga

SEQ ID NO: 300 - DNA, 1896 bp, synthetic construct
  source 1..1896: mol_type = other DNA; note = nucleotide sequence of SHC/HAC variant #115A7
atggctgagcagttggtggaagctccggcctacgcgcggacgctggatcgcgcggtggagtatctcctctcctgccaaaaggacgaaggctactggtgggggccgcttctgagcaacgtcacgatggaagcggagtacgtcctcttgtgccacattctcgatcgcgtcgatcgggatcgcatggagaagatccggcggtacctgttgcacgagcagcgcgaggacggcacgtgggccctgtacccgggtgggccgccggacctcgacacgaccatcgaggcgtacgtcgcgctcaagtatatcggcatgtcgcgcgacgaggagccgatgcagaaggcgctccggttcattcagagccagggcgggatcgagtcgtcgcgcgtgttcacgcggaggtggctggcgctggtgggagaatatccgtgggagaaggtgcccatggtcccgccggagatcatgttcctcggcaagcgcatgccgctcaacatctacgagtttggctcgtgggctcggacgaccgtcgtggcgctctcgattgtgatgagccgccagccggtgttcccgctgcccgagcgggcgcgcgtgcccgagctgtacgagaccgacgtgcctccgcgccggcgcggtgccaagggagggggtgggtggatcttcgacgcgctcgaccgggtgctgcacgggtatcagaagctgtcggtgcacccgttccgccgcgcggccgagatccgcgccttggactggttgctcgagcgccaggccggagacggcagctggggcgggattcagccgccttggttttacgcgctcatcgcgctcaagattctcgacaagacgcagcatccggcgttcatcaagggctgggaaggtctagagctgtacggcgtggagctggattacggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacaccccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacggcctgggcgctgatggcgctcatcgcgggcggcagggcggagtccgaggccgcgcgccgcggcgtgcaatacctcgtggagacgcagcgcccggacggcggctgggatgagccgtactacaccggcacgggcttcccaggggatttctacctcggctacaccatgtaccgccacgtgtttccgacgctcgcgctcggccgctacaagcaagccatcgagcgcaggtga

SEQ ID NO: 301 - protein, 685 aa, synthetic construct
  source 1..685: note = amino acid sequence of a GmoSHC variant
MSPADISTKSSSFQRLDNMLPEAVSSACDWLIDQQKPDGHWVGPLESNACMEAEWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATIMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQASNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGYPGYGVGQAIKLDDPSLPDRLLQGAELSRAFMLRYDLYRQYFPVMALSRARRMMKEDASAAA

SEQ ID NO: 302 - protein, 642 aa, synthetic construct
  source 1..642: note = amino acid sequence of TelSHC with W172G, P311V, F425Y, G609A mutations
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSGARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQACVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAYDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTAFPCHFYLKYHYYQQHFPLTALGRYARWRNLLAT

SEQ ID NO: 303 - protein, 720 aa, synthetic construct
  source 1..720: note = amino acid sequence of ZmoSHC1 with Q221S+222G+A368V + F486Y + G667A mutations
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFASGARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQVTNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAYDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYMEIDSIGKGPTTPSQTAWALMGLIAANRPEDYEAIAKGCHYLIDRQEQDGSWKEEEFTGTAFPGYGVGQTIKLDDPALSKRLLQGAELSRAFMLRYDFYRQFFPIMALSRAERL

SEQ ID NO: 304 - protein, 642 aa, synthetic construct
  source 1..642: note = amino acid sequence of TelSHC with W172G mutation (P1).
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSGARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQPCVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAFDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTGFPCHFYLKYHYYQQHFPLTALGRYARWRNLLAT

SEQ ID NO: 305 - protein, 631 aa, synthetic construct; identical to SEQ ID NO: 11
  source 1..631: note = amino acid sequence of AacSHC with W169G, G600M, M132R, A224V and I432T mutations

SEQ ID NO: 306 - protein, 631 aa, synthetic construct
  source 1..631: note = amino acid sequence of AacSHC with W169G, G600M, M132R and I432T mutations.
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 307 - protein, 685 aa, Gluconobacter morbifer
  source 1..685: note = strain: g707; note = EHH69691.1 squalene-hopene cyclase [Gluconobacter morbifer G707]
MSPADISTKSSSFQRLDNMLPEAVSSACDWLIDQQKPDGHWVGPVESNACMEAQWCLALWFLGQEDHPLRPRLAQALLEMQREDGSWGIYVGADHGDINTTVEAYAALRSMGYAADMPIMAKSAAWIQQKGGLRNVRVFTRYWLALIGEWPWDKTPNLPPEIIWLPDNFIFSIYNFAQWARATMMPLTILSARRPSRPLLPENRLDGLFPEGRENFDYELPVKGEEDLWGRFFRAADKGLHSLQSFPVRRFVPREAAIRHVIEWIIRHQDADGGWGGIQPPWIYGLMALSVEGYPLHHPVLAKAMDALNDPGWRRDKGDASWIQATNSPVWDTMLAVLALHDAGAEDRYSPQMDKAIGWLLDRQVRVKGDWSIKLPDTEPGGWAFEYANDKYPDTDDTAVALIALAGCRHRPEWRERDIEGAISRGVNWLLAMQSSSGGWGAFDKDNNRSILTKIPFCDFGEALDPPSVDVTAHVLEAFGLLGISRNHPSVQKALAYIRSEQERNGAWFGRWGVNYVYGTGAVLPALAAIGEDMTQPYIVRACDWLMSVQQENGGWGESCASYMDINAVGHGVATASQTAWALIGLLAAKRPKDREAIARGCQFLIERQEDGSWTEEEYTGTGFPGYGVGQAIKLDDPSLPDRLLQGAELSRAFMLRYDLYRQYFPVMALSRARRMMKEDASAAA

SEQ ID NO: 308 - protein, 658 aa, Zymomonas mobilis
  source 1..658: note = subspecies: mobilis; note = EER62728.1 squalene-hopene cyclase [Zymomonas mobilis subsp. mobilis ATCC 10988]
MTVSTSSAFHHSSLSDDVEPIIQKATRALLEKQHQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYWARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAIQKAVAWLKNIQNEDGGWGENCDSYALDYSGYEPMDSTASQTAWALLGLMAVGEANSEAVTKGINWLAQNQDEEGLWKEDYYSGGGFPRVFYLRYHGYSKYFPLWALARYRNLKKANQPIVHYGM

SEQ ID NO: 309 - protein, 714 aa, Komagataeibacter xylinus; identical to SEQ ID NO: 287
  source 1..714: note = strain: e25; note = AHI26287.1 squalene--hopene cyclase [Komagataeibacter xylinus E25]

SEQ ID NO: 310 - protein, 618 aa, Geobacillus; identical to SEQ ID NO: 283
  source 1..618: note = WP_031409036.1 MULTISPECIES: squalene--hopene cyclase [Geobacillus]

SEQ ID NO: 311 - protein, 680 aa, synthetic construct
  source 1..680: note = amino acid sequence of ScoSHC1 with W196G mutation (P1)
MTATTDGSTGASLRPLAASASDTDITIPAAAAGVPEAAARATRRATDFLLAKQDAEGWWKGDLETNVTMDAEDLLLRQFLGIQDEETTRAAALFIRGEQREDGTWATFYGGPGELSTTIEAYVALRLAGDSPEAPHMARAAEWIRSRGGIASARVFTRIWLALFGWWKWDDLPELPPELIYFPTWVPLNIYDFGCGARQTIVPLTIVSAKRPVRPAPFPLDELHTDPARPNPPRPLAPVASWDGAFQRIDKALHAYRKVAPRRLRRAAMNSAARWIIERQENDGCWGGIQPPAVYSVIALYLLGYDLEHPVMRAGLESLDRFAVWREDGARMIEACQSPVWDTCLATIALADAGVPEDHPQLVKASDWMLGEQIVRPGDWSVKRPGLPPGGWAFEFHNDNYPDIDDTAEVVLALRRVRHHDPERVEKAIGRGVRWNLGMQSKNGAWGAFDVDNTSAFPNRLPFCDFGEVIDPPSADVTAHVVEMLAVEGLAHDPRTRRGIQWLLDAQETDGSWFGRWGVNYVYGTGSVIPALTAAGLPTSHPAIRRAVRWLESVQNEDGGWGEDLRSYRYVREWSGRGASTASQTGWALMALLAAGERDSKAVERGVAWLAATQREDGSWDEPYFTGTGFPWDFSINYNLYRQVFPLTALGRYVHGEPFAKKPRAADAPAEAAPAEVKGS

SEQ ID NO: 312 - protein, 725 aa, synthetic construct
  source 1..725: note = amino acid sequence of ZmoSHC1 with W222G mutation (P1)
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQGARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQATNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYMEIDSIGKGPTTPSQTAWALMGLIAANRPEDYEAIAKGCHYLIDRQEQDGSWKEEEFTGTGFPGYGVGQTIKLDDPALSKRLLQGAELSRAFMLRYDFYRQFFPIMALSRAERLIDLNN

SEQ ID NO: 313 - protein, 658 aa, synthetic construct
  source 1..658: note = amino acid sequence of ZmoSHC2 with W177G mutation (P1)
MTVSTSSAFHHSPLSDDVEPIIQKATRALLEKQQQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYGARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAVQKAVAWLKTIQNEDGGWGENCDSYALDYSGYEPMDSTASQTAWALLGLMAVGEANSEAVTKGINWLAQNQDEEGLWKEDYYSGGGFPRVFYLRYHGYSKYFPLWALARYRNLKKANQPIVHYGM

SEQ ID NO: 314 - protein, 6 aa, synthetic construct
  source 1..6: note = the motif DXDDTA found in SHC/HAC
  VARIANT 2: note = X may be any amino acid.
DXDDTA

SEQ ID NO: 315 - protein, 631 aa, synthetic construct; identical to SEQ ID NO: 4
  source 1..631: note = amino acide sequence of AacSHC with W169G mutation

SEQ ID NO: 316 - protein, 631 aa, synthetic construct
  source 1..631: note = amino acide sequence of AacSHC with W169A mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 317 - protein, 631 aa, synthetic construct
  source 1..631: note = amino acide sequence of AacSHC with W169V mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 318 - protein, 631 aa, synthetic construct
  source 1..631: note = amino acide sequence of AacSHC with W169G and G600A mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 319 - protein, 631 aa, synthetic construct
  source 1..631: note = amino acide sequence of AacSHC with W169G and G600V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 320 - protein, 631 aa, synthetic construct
  source 1..631: note = amino acide sequence of AacSHC with W169G and G600L mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 321 - protein, 631 aa, synthetic construct
  source 1..631: note = amino acide sequence of AacSHC with W169G and G600I mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 322 - protein, 631 aa, synthetic construct; identical to SEQ ID NO: 5
  source 1..631: note = amino acide sequence of AacSHC with W169G and G600M mutations

SEQ ID NO: 323 - protein, 631 aa, synthetic construct
  source 1..631: note = amino acide sequence of AacSHC with W169A and G600A mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 324 - protein, 631 aa, synthetic construct
  source 1..631: note = amino acide sequence of AacSHC with W169A and G600V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 325 - protein, 631 aa, synthetic construct
  source 1..631: note = amino acide sequence of AacSHC with W169A and G600L mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 326 - protein, 631 aa, synthetic construct
  source 1..631: note = amino acide sequence of AacSHC with W169A and G600I mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 327 - protein, 631 aa, synthetic construct
  source 1..631: note = amino acide sequence of AacSHC with W169A and G600M mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 328 - protein, 631 aa, synthetic construct
  source 1..631: note = amino acide sequence of AacSHC with W169V and G600A mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 329 - protein, 631 aa, synthetic construct
  source 1..631: note = amino acide sequence of AacSHC with W169V and G600V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 330 - protein, 631 aa, synthetic construct
  source 1..631: note = amino acide sequence of AacSHC with W169V and G600L mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 331 - protein, 631 aa, synthetic construct
  source 1..631: note = amino acide sequence of AacSHC with W169V and G600I mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 332 - protein, 631 aa, synthetic construct
  source 1..631: note = amino acide sequence of AacSHC with W169V and G600M mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 333 - protein, 631 aa, synthetic construct
  source 1..631: note = amino acide sequence of AacSHC with W169G and G600A and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 334 - protein, 631 aa, synthetic construct
  source 1..631: note = amino acide sequence of AacSHC with W169G and G600V and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 335 - protein, 631 aa, synthetic construct
  source 1..631: note = amino acide sequence of AacSHC with W169G and G600L and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 336 - protein, 631 aa, synthetic construct
  source 1..631: note = amino acide sequence of AacSHC with W169G and G600I and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 337 - protein, 631 aa, synthetic construct; identical to SEQ ID NO: 6
  source 1..631: note = amino acide sequence of AacSHC with W169G and G600M and A306V mutations

SEQ ID NO: 338 - protein, 631 aa, synthetic construct
  source 1..631: note = amino acide sequence of AacSHC with W169A and G600A and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 339 - protein, 631 aa, synthetic construct
  source 1..631: note = amino acide sequence of AacSHC with W169A and G600V and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 340 - protein, 631 aa, synthetic construct
  source 1..631: note = amino acide sequence of AacSHC with W169A and G600L and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 341 - protein, 631 aa, synthetic construct
  source 1..631: note = amino acide sequence of AacSHC with W169A and G600I and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 342 - protein, 631 aa, synthetic construct
  source 1..631: note = amino acide sequence of AacSHC with W169A and G600M and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 343 - protein, 631 aa, synthetic construct
  source 1..631: note = amino acide sequence of AacSHC with W169V and G600A and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 344 - protein, 631 aa, synthetic construct
  source 1..631: note = amino acide sequence of AacSHC with W169V and G600V and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 345 - protein, 631 aa, synthetic construct
  source 1..631: note = amino acide sequence of AacSHC with W169V and G600L and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 346 - protein, 631 aa, synthetic construct
  source 1..631: note = amino acide sequence of AacSHC with W169V and G600I and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 347 - protein, 631 aa, synthetic construct
  source 1..631: note = amino acide sequence of AacSHC with W169V and G600M and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 348 - protein, 631 aa, synthetic construct; identical to SEQ ID NO: 3
  source 1..631: note = amino acide sequence of AacSHC with M132R and A224V and I432T mutations

SEQ ID NO: 349 - protein, 1896 aa, synthetic construct
  source 1..1896: note = nucleotide sequence coding for wild type AacSHC
ATGGCGGAACAGCTGGTGGAAGCGCCGGCGTATGCGCGCACCCTGGATCGCGCGGTGGAATATCTGCTGAGCTGCCAGAAAGATGAAGGCTATTGGTGGGGCCCGCTGCTGAGCAACGTGACCATGGAAGCGGAATATGTGCTGCTGTGCCATATTCTGGATCGCGTGGATCGCGATCGCATGGAAAAAATTCGCCGCTATCTGCTGCATGAACAGCGCGAAGATGGCACCTGGGCGCTGTATCCGGGCGGCCCGCCGGATCTGGATACCACCATTGAAGCGTATGTGGCGCTGAAATATATTGGCATGAGCCGCGATGAAGAACCGATGCAGAAAGCGCTGCGCTTTATTCAGAGCCAGGGCGGCATTGAAAGCAGCCGCGTGTTTACCCGCATGTGGCTGGCGCTGGTGGGCGAATATCCGTGGGAAAAAGTGCCGATGGTGCCGCCGGAAATTATGTTTCTGGGCAAACGCATGCCGCTGAACATTTATGAATTTGGCAGCTGGGCGCGCGCGACCGTGGTGGCGCTGAGCATTGTGATGAGCCGCCAGCCGGTGTTTCCGCTGCCGGAACGCGCGCGCGTGCCGGAACTGTATGAAACCGATGTGCCGCCGCGCCGCCGCGGCGCGAAAGGCGGCGGCGGCTGGATTTTTGATGCGCTGGATCGCGCGCTGCATGGCTATCAGAAACTGAGCGTGCATCCGTTTCGCCGCGCGGCGGAAATTCGCGCGCTGGATTGGCTGCTGGAACGCCAGGCGGGCGATGGCAGCTGGGGCGGCATTCAGCCGCCGTGGTTTTATGCGCTGATTGCGCTGAAAATTCTGGATATGACCCAGCATCCGGCGTTTATTAAAGGCTGGGAAGGCCTGGAACTGTATGGCGTGGAACTGGATTATGGCGGCTGGATGTTTCAGGCGAGCATTAGCCCGGTGTGGGATACCGGCCTGGCGGTGCTGGCGCTGCGCGCGGCGGGCCTGCCGGCGGATCATGATCGCCTGGTGAAAGCGGGCGAATGGCTGCTGGATCGCCAGATTACCGTGCCGGGCGATTGGGCGGTGAAACGCCCGAACCTGAAACCGGGCGGCTTTGCGTTTCAGTTTGATAACGTGTATTATCCGGATGTGGATGATACCGCGGTGGTGGTGTGGGCGCTGAACACCCTGCGCCTGCCGGATGAACGCCGCCGCCGCGATGCGATGACCAAAGGCTTTCGCTGGATTGTGGGCATGCAGAGCAGCAACGGCGGCTGGGGCGCGTATGATGTGGATAACACCAGCGATCTGCCGAACCATATTCCGTTTTGCGATTTTGGCGAAGTGACCGATCCGCCGAGCGAAGATGTGACCGCGCATGTGCTGGAATGCTTTGGCAGCTTTGGCTATGATGATGCGTGGAAAGTGATTCGCCGCGCGGTGGAATATCTGAAACGCGAACAGAAACCGGATGGCAGCTGGTTTGGCCGCTGGGGCGTGAACTATCTGTATGGCACCGGCGCGGTGGTGAGCGCGCTGAAAGCGGTGGGCATTGATACCCGCGAACCGTATATTCAGAAAGCGCTGGATTGGGTGGAACAGCATCAGAACCCGGATGGCGGCTGGGGCGAAGATTGCCGCAGCTATGAAGATCCGGCGTATGCGGGCAAAGGCGCGAGCACCCCGAGCCAGACCGCGTGGGCGCTGATGGCGCTGATTGCGGGCGGCCGCGCGGAAAGCGAAGCGGCGCGCCGCGGCGTGCAGTATCTGGTGGAAACCCAGCGCCCGGATGGCGGCTGGGATGAACCGTATTATACCGGCACCGGGTTCCCGGGCGATTTTTATCTGGGCTATACCATGTATCGCCATGTGTTTCCGACCCTGGCGCTGGGCCGCTATAAACAGGCGATTGAACGCCGCTAA

SEQ ID NO: 350 - protein, 631 aa, synthetic construct
  source 1..631: note = amino acide sequence of AacSHC with W169G and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSGARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 351 - protein, 631 aa, synthetic construct
  source 1..631: note = amino acide sequence of AacSHC with W169A and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSAARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 352 - protein, 631 aa, synthetic construct
  source 1..631: note = amino acide sequence of AacSHC with W169V and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSVARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 353 - protein, 725 aa, synthetic construct
  source 1..725: note = SEQ ID NO: 13 with the substitutions W222G and G667M (or ZmoSHC1 P2 variant)
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQGARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQATNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYMEIDSIGKGPTTPSQTAWALMGLIAANRPEDYEAIAKGCHYLIDRQEQDGSWKEEEFTGTMFPGYGVGQTIKLDDPALSKRLLQGAELSRAFMLRYDFYRQFFPIMALSRAERLIDLNN

SEQ ID NO: 354 - protein, 725 aa, synthetic construct
  source 1..725: note = SEQ ID NO: 13 with the substitutions W222G, A368V and G667M (also called the ZmoSHC1 P3 variant).
MGIDRMNSLSRLLMKKIFGAEKTSYKPASDTIIGTDTLKRPNRRPEPTAKVDKTIFKTMGNSLNNTLVSACDWLIGQQKPDGHWVGAVESNASMEAEWCLALWFLGLEDHPLRPRLGNALLEMQREDGSWGVYFGAGNGDINATVEAYAALRSLGYSADNPVLKKAAAWIAEKGGLKNIRVFTRYWLALIGEWPWEKTPNLPPEIIWFPDNFVFSIYNFAQGARATMVPIAILSARRPSRPLRPQDRLDELFPEGRARFDYELPKKEGIDLWSQFFRTTDRGLHWVQSNLLKRNSLREAAIRHVLEWIIRHQDADGGWGGIQPPWVYGLMALHGEGYQLYHPVMAKALSALDDPGWRHDRGESSWIQVTNSPVWDTMLALMALKDAKAEDRFTPEMDKAADWLLARQVKVKGDWSIKLPDVEPGGWAFEYANDRYPDTDDTAVALIALSSYRDKEEWQKKGVEDAITRGVNWLIAMQSECGGWGAFDKDNNRSILSKIPFCDFGESIDPPSVDVTAHVLEAFGTLGLSRDMPVIQKAIDYVRSEQEAEGAWFGRWGVNYIYGTGAVLPALAAIGEDMTQPYITKACDWLVAHQQEDGGWGESCSSYMEIDSIGKGPTTPSQTAWALMGLIAANRPEDYEAIAKGCHYLIDRQEQDGSWKEEEFTGTMFPGYGVGQTIKLDDPALSKRLLQGAELSRAFMLRYDFYRQFFPIMALSRAERLIDLNN

SEQ ID NO: 355 - protein, 658 aa, synthetic construct
  source 1..658: note = SEQ ID NO: 15 with the substitutions W177G and G619M (also called the ZmoSHC2 P2 variant
MTVSTSSAFHHSPLSDDVEPIIQKATRALLEKQQQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYGARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAVQKAVAWLKTIQNEDGGWGENCDSYALDYSGYEPMDSTASQTAWALLGLMAVGEANSEAVTKGINWLAQNQDEEGLWKEDYYSGGMFPRVFYLRYHGYSKYFPLWALARYRNLKKANQPIVHYGM

SEQ ID NO: 356 - protein, 658 aa, synthetic construct
  source 1..658: note = SEQ ID NO: 15 with the substitutions W177G, A321V and G619M (also called the ZmoSHC2 P3 variant
MTVSTSSAFHHSPLSDDVEPIIQKATRALLEKQQQDGHWVFELEADATIPAEYILLKHYLGEPEDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYGARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQVCLSPIWDTALVAHAMLEVGGDEAEKSAISALSWLKPQQILDVKGDWAWRRPDLRPGGWAFQYRNDYYPDVDDTAVVTMAMDRAAKLSDLHDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAVQKAVAWLKTIQNEDGGWGENCDSYALDYSGYEPMDSTASQTAWALLGLMAVGEANSEAVTKGINWLAQNQDEEGLWKEDYYSGGMFPRVFYLRYHGYSKYFPLWALARYRNLKKANQPIVHYGM

SEQ ID NO: 357 - protein, 680 aa, synthetic construct
  source 1..680: note = SEQ ID NO: 32 with the substitutions W196G and G629M (also called the ScoSHC P2 variant)
MTATTDGSTGASLRPLAASASDTDITIPAAAAGVPEAAARATRRATDFLLAKQDAEGWWKGDLETNVTMDAEDLLLRQFLGIQDEETTRAAALFIRGEQREDGTWATFYGGPGELSTTIEAYVALRLAGDSPEAPHMARAAEWIRSRGGIASARVFTRIWLALFGWWKWDDLPELPPELIYFPTWVPLNIYDFGCGARQTIVPLTIVSAKRPVRPAPFPLDELHTDPARPNPPRPLAPVASWDGAFQRIDKALHAYRKVAPRRLRRAAMNSAARWIIERQENDGCWGGIQPPAVYSVIALYLLGYDLEHPVMRAGLESLDRFAVWREDGARMIEACQSPVWDTCLATIALADAGVPEDHPQLVKASDWMLGEQIVRPGDWSVKRPGLPPGGWAFEFHNDNYPDIDDTAEVVLALRRVRHHDPERVEKAIGRGVRWNLGMQSKNGAWGAFDVDNTSAFPNRLPFCDFGEVIDPPSADVTAHVVEMLAVEGLAHDPRTRRGIQWLLDAQETDGSWFGRWGVNYVYGTGSVIPALTAAGLPTSHPAIRRAVRWLESVQNEDGGWGEDLRSYRYVREWSGRGASTASQTGWALMALLAAGERDSKAVERGVAWLAATQREDGSWDEPYFTGTMFPWDFSINYNLYRQVFPLTALGRYVHGEPFAKKPRAADAPAEAAPAEVKGS

SEQ ID NO: 358 - protein, 680 aa, synthetic construct
  source 1..680: note = SEQ ID NO: 32 with the substitutions W196G, A335V and G629M (also called the ScoSHC P3 variant
MTATTDGSTGASLRPLAASASDTDITIPAAAAGVPEAAARATRRATDFLLAKQDAEGWWKGDLETNVTMDAEDLLLRQFLGIQDEETTRAAALFIRGEQREDGTWATFYGGPGELSTTIEAYVALRLAGDSPEAPHMARAAEWIRSRGGIASARVFTRIWLALFGWWKWDDLPELPPELIYFPTWVPLNIYDFGCGARQTIVPLTIVSAKRPVRPAPFPLDELHTDPARPNPPRPLAPVASWDGAFQRIDKALHAYRKVAPRRLRRAAMNSAARWIIERQENDGCWGGIQPPAVYSVIALYLLGYDLEHPVMRAGLESLDRFAVWREDGARMIEVCQSPVWDTCLATIALADAGVPEDHPQLVKASDWMLGEQIVRPGDWSVKRPGLPPGGWAFEFHNDNYPDIDDTAEVVLALRRVRHHDPERVEKAIGRGVRWNLGMQSKNGAWGAFDVDNTSAFPNRLPFCDFGEVIDPPSADVTAHVVEMLAVEGLAHDPRTRRGIQWLLDAQETDGSWFGRWGVNYVYGTGSVIPALTAAGLPTSHPAIRRAVRWLESVQNEDGGWGEDLRSYRYVREWSGRGASTASQTGWALMALLAAGERDSKAVERGVAWLAATQREDGSWDEPYFTGTMFPWDFSINYNLYRQVFPLTALGRYVHGEPFAKKPRAADAPAEAAPAEVKGS

SEQ ID NO: 359 - protein, 642 aa, synthetic construct
  source 1..642: note = SEQ ID NO: 23 with the substitutions W172G and G609M (also called the TelSHC P2 variant
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSGARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQPCVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAFDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTMFPCHFYLKYHYYQQHFPLTALGRYARWRNLLAT

SEQ ID NO: 360 - protein, 642 aa, synthetic construct
  source 1..642: note = SEQ ID NO: 23 with the substitutions W172G, A311V and G609M (also called the TelSHC P3 variant
MPTSLATAIDPKQLQQAIRASQDFLFSQQYAEGYWWAELESNVTMTAEVILLHKIWGTEQRLPLAKAEQYLRNHQRDHGGWELFYGDGGDLSTSVEAYMGLRLLGVPETDPALVKARQFILARGGISKTRIFTKLHLALIGCYDWRGIPSLPPWIMLLPEGSPFTIYEMSSGARSSTVPLLIVMDRKPVYGMDPPITLDELYSEGRANVVWELPRQGDWRDVFIGLDRVFKLFETLNIHPLREQGLKAAEEWVLERQEASGDWGGIIPAMLNSLLALRALDYAVDDPIVQRGMAAVDRFAIETETEYRVQVCVSPVWDTALVMRAMVDSGVAPDHPALVKAGEWLLSKQILDYGDWHIKNKKGRPGGWAFEFENRFYPDVDDTAVVVMALHAVTLPNENLKRRAIERAVAWIASMQCRPGGWAAFDVDNDQDWLNGIPYGDLKAMIDPNTADVTARVLEMVGRCQLAFDRVALDRALAYLRNEQEPEGCWFGRWGVNYLYGTSGVLTALSLVAPRYDRWRIRRAAEWLMQCQNADGGWGETCWSYHDPSLKGKGDSTASQTAWAIIGLLAAGDATGDYATEAIERGIAYLLETQRPDGTWHEDYFTGTMFPCHFYLKYHYYQQHFPLTALGRYARWRNLLAT

SEQ ID NO: 361 - protein, 631 aa, synthetic construct
  source 1..631: note = amino acide sequence of AacSHC with T77A mutation
MAEQLVEAPAYARTLDRAVEYLLSCQKDEGYWWGPLLSNVTMEAEYVLLCHILDRVDRDRMEKIRRYLLHEQREDGAWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 362 - protein, 631 aa, synthetic construct
  source 1..631: note = amino acide sequence of AacSHC with I92V mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTVEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 363 - protein, 631 aa, synthetic construct
  source 1..631: note = amino acide sequence of AacSHC with F129L mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVLTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 364 - protein, 631 aa, synthetic construct
  source 1..631: note = amino acide sequence of AacSHC with M132R mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 365 - protein, 631 aa, synthetic construct
  source 1..631: note = amino acide sequence of AacSHC with A224V mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 366 - protein, 631 aa, synthetic construct
  source 1..631: note = amino acide sequence of AacSHC with I432T mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 367 - protein, 631 aa, synthetic construct
  source 1..631: note = amino acide sequence of AacSHC with Q579H mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVHYLVETQRPDGGWDEPYYTGTGFPGDFYLGYTMYRHVFPTLALGRYKQAIERR

SEQ ID NO: 368 - protein, 631 aa, synthetic construct
  source 1..631: note = amino acide sequence of AacSHC with F601Y mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGYPGDFYLGYTMYRHVFPTLALGRYKQAIERR

SEQ ID NO: 369 - protein, 631 aa, synthetic construct
  source 1..631: note = amino acide sequence of AacSHC with M132R+I432Tmutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 370 - protein, 631 aa, synthetic construct; identical to SEQ ID NO: 368
  source 1..631: note = amino acide sequence of AacSHC with F601Y mutation

SEQ ID NO: 371 - protein, 631 aa, synthetic construct
  source 1..631: note = amino acide sequence of AacSHC with T77A+I92V+F129L mutations
MAEQLVEAPAYARTLDRAVEYLLSCQKDEGYWWGPLLSNVTMEAEYVLLCHILDRVDRDRMEKIRRYLLHEQREDGAWALYPGGPPDLDTTVEAYVALKYIGMSRDEEPMQKALRFIQSQGGIESSRVLTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 372 - protein, 631 aa, synthetic construct
  source 1..631: note = amino acide sequence of AacSHC with Q579H+F601Y mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVHYLVETQRPDGGWDEPYYTGTGYPGDFYLGYTMYRHVFPTLALGRYKQAIERR

SEQ ID NO: 373 - protein, 631 aa, synthetic construct; identical to SEQ ID NO: 363
  source 1..631: note = amino acide sequence of AacSHC with F129L mutation

SEQ ID NO: 374 - protein, 631 aa, synthetic construct
  source 1..631: note = amino acide sequence of AacSHC with F129L+F601Y mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVLTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGYPGDFYLGYTMYRHVFPTLALGRYKQAIERR

SEQ ID NO: 375 - protein, 631 aa, synthetic construct
  source 1..631: note = amino acide sequence of AacSHC with F129L+M132R+I432T mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVLTRRWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 376 - protein, 631 aa, synthetic construct
  source 1..631: note = amino acide sequence of AacSHC with M132R+I432T +F601Y mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGYPGDFYLGYTMYRHVFPTLALGRYKQAIERR

SEQ ID NO: 377 - protein, 631 aa, synthetic construct
  source 1..631: note = amino acide sequence of AacSHC with F129L+M132R+I432T+F601Y mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVLTRRWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGYPGDFYLGYTMYRHVFPTLALGRYKQAIERR

SEQ ID NO: 378 - protein, 631 aa, synthetic construct
  source 1..631: note = amino acide sequence of AacSHC with F605W mutation
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDWYLGYTMYRHVFPTLALGRYKQAIERR

SEQ ID NO: 379 - protein, 631 aa, synthetic construct
  source 1..631: note = amino acide sequence of SHC/HAC enzyme variant #49
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSWARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNLTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYRHVFPTLALGRYKQAIERR

SEQ ID NO: 380 - protein, 631 aa, synthetic construct; identical to SEQ ID NO: 47
  source 1..631: note = amino acide sequence of SHC/HAC enzyme variant #66

SEQ ID NO: 381 - protein, 631 aa, synthetic construct; identical to SEQ ID NO: 48
  source 1..631: note = amino acide sequence of SHC/HAC enzyme variant #110B8

SEQ ID NO: 382 - protein, 631 aa, synthetic construct; identical to SEQ ID NO: 49
  source 1..631: note = amino acide sequence of SHC/HAC enzyme variant #90C7

SEQ ID NO: 383 - protein, 631 aa, synthetic construct; identical to SEQ ID NO: 50
  source 1..631: note = amino acide sequence of SHC/HAC enzyme variant #115A7

SEQ ID NO: 384 - protein, 725 aa, Zymomonas mobilis; identical to SEQ ID NO: 13
  source 1..725: note = amino acid sequence of wild-type ZmoSHC1

SEQ ID NO: 385 - protein, 631 aa, synthetic construct
  source 1..631: note = amino acid sequence of Aac 215G2 SHC/HAC enzyme variant with W169G and A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGYTMYRHVFPTLALGRYKQAIERR

SEQ ID NO: 386 - protein, 631 aa, synthetic construct
  source 1..631: note = amino acid sequence of Aac #65 SHC/HAC enzyme variant + W169G+A306V mutations
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRRWLALVGEYPWEKVPMVPPEIMFLGKRMPLNIYEFGSGARATVVALSIVMSRQPVFPLPERARVPELYETDVPPRRRGAKGGGGWIFDALDRVLHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHTPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTTWALMALIAGGRAESEAARRGVQYLVETQRPDGGWDEPYYTGTGFPGDFYLGYTMYSHVFPTLALGRYKQAIERR

(19) United States
(12) Patent Application Publication
(10) Pub. No.: US 2026/0043058 A1
(43) Pub. Date: Feb. 12, 2026

(54) SHC ENZYMES AND ENZYME VARIANTS

(71) Applicant: GIVAUDAN SA, VERNIER (CH)

(72) Inventors: Eric EICHHORN, Kemptthal (CH); Bernhard HAUER, Fussgoenheim (DE); Andreas SCHNEIDER, Leinfelden-Echterdingen (DE)

(21) Appl. No.: 18/847,838

(22) PCT Filed: Mar. 17, 2023
(86) PCT No.: PCT/EP2023/056842

§ 371 Date: Sep. 17, 2024

(30) Foreign Application Priority Data
Mar. 17, 2022 (EP) 22162848.0
Apr. 14, 2022 (EP) 22168607.4

(51) Int. Cl. C12P 17/04; C12N 9/10; C12N 9/90

(52) U.S. Cl. CPC C12P 17/04; C12N 9/1029; C12N 9/90; C12Y 203/01048; C12Y 504/99017

(57) ABSTRACT

Squalene Hopene Cyclase (SHC) enzymes and variants thereof and their uses for making (−)-Ambrox from homofarnesol and Ambra oxide from bishomofarnesol and the like.

### TECHNICAL FIELD

The present invention relates generally to SHC/HAC enzymes and variants thereof. The present invention further relates to the various uses of the SHC/HAC enzymes and variants thereof, for example to enzymatically convert (3E,7E)-homofarnesol (EEH) to (−)-Ambrox or to enzymatically convert E,E-bishomofarnesol (BisEEH) to Ambra oxide and the like. The present invention also relates to the products of the enzymatic reactions, for example the (−)-Ambrox or Ambra oxide made using the SHC/HAC enzymes and variants thereof, and the various uses of said products.

### BACKGROUND

Squalene Hopene Cyclases (SHCs) are membrane-bound enzymes which act as biocatalysts for the cyclisation of the linear triterpenoid squalene to hopene and hopanol.

A number of wild-type and variant SHC enzymes from a variety of bacteria have been demonstrated to be useful to convert (3E,7E)-homofarnesol to (−)-Ambrox (see, for example, WO 2016/170099; WO 2018/157021; Neumann & Simon 1986, Biol Chem Hoppe-Seyler 367, 723-729; JP2009060799; Seckler & Poralla 1986, Biochem Biophys Act 356-363; Ochs et al 1990, J Bacteriol 174, 298-302; WO 2010/139719; U.S. Pat. No. 8,759,043; WO 2012/066059; Seitz et al 2012, J Molecular Catalysis B: Enzymatic 84, 72-77; and Seitz 2012 PhD thesis (http://elib.uni-stuttgart.de/handle/1 1682/1400), the contents of which are incorporated herein by reference). It is desirable to provide new and improved methods for making (−)-Ambrox, for example using new SHC enzymes or enzyme variants. It is also desirable to provide new and improved methods for cyclizing other substrates, for example to form compounds useful in or as fragrances.

### SUMMARY

In a first aspect, there is provided a process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant, wherein the SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295,296, 307, 308, 309,310, 361, 362, 363, 364, 365, 366, 367, 368, 369, 370, 371, 372, 373, 374, 375, 376, 377, 378, 379, 380, 381, 382 or 383 and wherein the SHC/HAC enzyme variant amino acid sequence has amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382, or 383 at positions corresponding to positions W169, A306 and/or G600 of SEQ ID NO: 1, 2, or 3 or at a position in an amino acid sequence of a wild type SHC/HAC enzyme corresponding to W169, A306 and/or G600 of SEQ ID NO:1, 2, or 3.

In an embodiment, there is provided a process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant, wherein the SHC/HAC enzyme variant has an amino acid sequence having at least 30.0% identity or similarity to SEQ ID NO: 1, 13, 15, 23, 32, and wherein the SHC/HAC enzyme variant amino acid sequence has amino acid alterations relative to SEQ ID NO: 1 at positions corresponding to positions W169, A306 and/or G600 of SEQ ID NO: 1 or at a position in an amino acid sequence of a wild type SHC/HAC enzyme corresponding to W169, A306 and/or G600 of SEQ ID NO: 1.

In an embodiment, there is provided a process for preparing Ambra oxide or a mixture comprising Ambra oxide, the process comprising enzymatically converting (2,E)-Bishomofarnesol (BisEEH) or a mixture of isomers of bishomofarnesol comprising BisEEH to Ambra oxide or a mixture comprising Ambra oxide using a SHC/HAC enzyme variant, wherein the SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309,310, 361, 362, 363, 364, 365, 366, 367, 368, 369, 370, 371, 372, 373, 374, 375, 376, 377, 378, 379, 380, 381, 382, or 383 and wherein the SHC/HAC enzyme variant amino acid sequence has amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382, or 383 at positions corresponding to positions W169, A306 and/or G600 of SEQ ID NO: 1, 2, or 3 or at a position in an amino acid sequence of a wild type SHC/HAC enzyme corresponding to W169, A306 and/or G600 of SEQ ID NO:1, 2, or 3.

In an embodiment, there is provided a process for preparing Ambra oxide or a mixture comprising Ambra oxide, the process comprising enzymatically converting (2,E)-Bishomofarnesol (BisEEH) or a mixture of isomers of bishomofarnesol comprising BisEEH to Ambra oxide or a mixture comprising Ambra oxide using a SHC/HAC enzyme variant, wherein the SHC/HAC enzyme variant has an amino acid sequence having at least 30.0% identity or similarity to SEQ ID NO: 1, 13, 15, 23, 32, and wherein the SHC/HAC enzyme variant amino acid sequence has amino acid alterations relative to SEQ ID NO: 1 at positions corresponding to positions W169, A306 and/or G600 of SEQ ID NO: 1 or at a position in an amino acid sequence of a wild type SHC/HAC enzyme corresponding to W169, A306 and/or G600 of SEQ ID NO: 1.

In an embodiment, there is provided a process wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, A or V or a functional equivalent thereof, preferably wherein the W at position 169 is replaced by G,
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by A, V, L, I or M or a functional equivalent thereof.

In an embodiment, there is provided a process wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G.

In an embodiment, there is provided a process wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, and
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V.

In an embodiment, there is provided a process wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, and
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by M.

In an embodiment, there is provided a process wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G,
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V, and
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by M.

In an embodiment, there is provided a process wherein:
the amino acid at position 168 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 is S or the amino acid at a position in an amino acid sequence of a wild type SHC corresponding to 168 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 is S.

In an embodiment, there is provided a process as defined above, wherein the amino acid at position 168 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is S (or the amino acid at a position in an amino acid sequence of a wild type SHC corresponding to 168 of SEQ ID NO:1) is S.

In an embodiment, there is provided a process as defined above, wherein
the SHC/HAC enzyme variant has an amino acid sequence having at least 70.0% identity to SEQ ID NO: 1, 4, 5, 6 or 350 and has at least one of the following mutations: W169G, A306V and G600M or
the SHC/HAC enzyme variant has an amino acid sequence having at least 70.0% identity to SEQ ID NO: 7, 8, 9 or 386 and has the following mutations: M132R, A224V, 1432T, A557T, R613S, and has at least one of the following mutations: W169G, A306V and G600M or
the SHC/HAC enzyme variant has an amino acid sequence having at least 70.0% identity to SEQ ID NO: 10, 11, 12 or 385 and has the following mutations: M132R, A224V, 1432T and has at least one of the following mutations: W169G, A306V and G600M.

In an embodiment, there is provided a process wherein the W at position 169 is replaced by G.

In an embodiment, there is provided a process wherein:
the W at position 169 is replaced by G, and the A at position is replaced by V.

In an embodiment, there is provided a process wherein:
the W at position 169 is replaced by G, and the G at position 600 is replaced by M.

In an embodiment, there is provided a process wherein:
the W at position 169 is replaced by G, the A at position 306 is replaced by V, and the G at position 600 is replaced by M.

In an embodiment, there is provided a process as defined above, wherein
the SHC/HAC enzyme variant has an amino acid sequence having at least 70.0% identity to SEQ ID NO: 23, 304, 359 or 360 and has at least one of the following mutations: W172G, A311V and G609M (Tel SHC/HAC variant) or
the SHC/HAC enzyme variant has an amino acid sequence having at least 70.0% identity to SEQ ID NO: 32, 311, 357, or 358 and has the following mutations: W196G, A335V and G629M (Sco SHC/HAC variant) or
the SHC/HAC enzyme variant has an amino acid sequence having at least 70.0% identity to SEQ ID NO:13, 312, 353 or 354 and has at least one of the following mutations: W222G, A368V and G667M (Zmo SHC1 variant) or
the SHC/HAC enzyme variant has an amino acid sequence having at least 70.0% identity to SEQ ID NO: 15, 313, 355 or 356 and has at least one of the following mutations: W177G, A321V and G619M (Zmo SHC2 variant).

In an embodiment, there is provided a process wherein:
the SHC/HAC enzyme variant has an amino acid sequence having at least 70.0% identity to SEQ ID NO: 23, 304, 359 or 360 and has a W172G mutation.

In an embodiment, there is provided a process wherein:
the SHC/HAC enzyme variant has an amino acid sequence having at least 70.0% identity to SEQ ID NO: 23, 304, 359 or 360 and has W172G and A311V mutations.

In an embodiment, there is provided a process wherein:
the SHC/HAC enzyme variant has an amino acid sequence having at least 70.0% identity to SEQ ID NO: 23, 304, 359 or 360 and has W172G and G609M mutations.

In an embodiment, there is provided a process wherein:
the SHC/HAC enzyme variant has an amino acid sequence having at least 70.0% identity to SEQ ID NO: 23, 304, 359 or 360 and has W172G, A311V and G609M mutations.

In an embodiment, there is provided a process wherein:
the SHC/HAC enzyme variant has an amino acid sequence having at least 70.0% identity to SEQ ID NO: 32, 311, 357, or 358 and has a W196G mutation.

In an embodiment, there is provided a process wherein:
wherein the SHC/HAC enzyme variant has an amino acid sequence having at least 70.0% identity to SEQ ID NO: 32, 311, 357, or 358 and has W196G and A335V mutations.

In an embodiment, there is provided a process wherein:
the SHC/HAC enzyme variant has an amino acid sequence having at least 70.0% identity to SEQ ID NO: 32, 311, 357, or 358 and has W196G and G629M mutations.

In an embodiment, there is provided a process wherein:
the SHC/HAC enzyme variant has an amino acid sequence having at least 70.0% identity to SEQ ID NO: 32, 311, 357, or 358 and has W196G, A335V and G629M mutations.

In an embodiment, there is provided a process wherein:
the SHC/HAC enzyme variant has an amino acid sequence having at least 70.0% identity to SEQ ID NO:13, 312, 353 or 354 and has W222G mutation.

In an embodiment, there is provided a process wherein:
the SHC/HAC enzyme variant has an amino acid sequence having at least 70.0% identity to SEQ ID NO:13, 312, 353 or 354 and has W222G and A368V mutations.

In an embodiment, there is provided a process wherein:
the SHC/HAC enzyme variant has an amino acid sequence having at least 70.0% identity to SEQ ID NO:13, 312, 353 or 354 and has W222G and G667M mutations.

In an embodiment, there is provided a process wherein:
the SHC/HAC enzyme variant has an amino acid sequence having at least 70.0% identity to SEQ ID NO:13, 312, 353 or 354 and has W222G, A368V and G667M mutations.

In an embodiment, there is provided a process wherein:
the SHC/HAC enzyme variant has an amino acid sequence having at least 70.0% identity to SEQ ID NO: 15, 313, 355 or 356 and has W177G mutation.

In an embodiment, there is provided a process wherein:
the SHC/HAC enzyme variant has an amino acid sequence having at least 70.0% identity to SEQ ID NO: 15, 313, 355 or 356 and has W177G and A321V mutations.

In an embodiment, there is provided a process wherein:
the SHC/HAC enzyme variant has an amino acid sequence having at least 70.0% identity to SEQ ID NO: 15, 313, 355 or 356 and has W177G and G619M mutation.

In an embodiment, there is provided a process wherein:
the SHC/HAC enzyme variant has an amino acid sequence having at least 70.0% identity to SEQ ID NO: 15, 313, 355 or 356 and has W177G, A321V and G619M mutations.

In an embodiment, there is provided a process as defined above, wherein the SHC/AHC enzyme variant has an amino acid sequence comprising or consisting of or essentially consisting of SEQ ID NO: 4, 5, 6, 7, 8, 9, 10, 11, 12, 305, 306, 304, 302, 311, 312, 313, 353, 354, 355, 356, 357, 358, 359, 360, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 350, 351, or 352.

In an embodiment, there is provided a process as defined above, wherein, the SHC/HAC enzyme variant disclosed herein exhibits:
an increased substrate specificity for EEH (or for BisEEH) when homofarnesol or bis-homofarnesol substrates are used,
an increased product selectivity for (−)-Ambrox (or Ambra oxide) when homofarnesol or bis-homofarnesol substrates are used,
an increased specificity for a particular isomer of substrates when substrates other than homofarnesol or bis-homofarnesol are used (eg ethyl-homofarnesol, hydroxyfarnesylacetone and the like—see, for example WO2021/110858 and WO2021/209482 respectively),
an increased productivity and/or
an increased degree of conversion of EEH (or BisEEH) as well as an increased conversion rate of EEH (or BisEEH) over the first 3 to 6 hours (or over the first 4, 5, or 6 hours) of the reaction, compared to the reference SHC enzyme (e.g. wild-type SHC such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or 215G2 AacSHC or SHC #65 or a parent SHC enzyme the variant derives from such as those represented by SEQ ID NO:2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383).

In an embodiment, there is provided a process as defined above wherein the enzymatic conversion takes place at a temperature in the range of about 30° C. to about 50° C., for example from about 40° to about 50° C., and/or at a pH in the range of about 5 to about 6.

In an embodiment, there is provided a process as defined above, wherein the process comprises culturing a recombinant host cell that produces the SHC/HAC enzyme variant.

In an embodiment, there is provided a process as defined above, wherein the recombinant host cells comprise a nucleic acid sequence selected from SEQ ID NO: 38, 39, 40, 41, 42, 43, 44, 45 or 46.

In an embodiment, there is provided a process as defined above, wherein the mixture of isomers of homofarnesol comprising EEH is an EE:EZ isomer mixture, preferably wherein the EE:EZ isomer mixture is in a weight ratio of 80:20.

In an embodiment, there is provided a process as defined above, wherein the weight ratio of the recombinant host cell producing the SHC/HAC enzyme variant to EEH or a mixture of isomers of homofarnesol comprising EEH in an EE:EZ isomer mixture in a weight ratio of 80:20 is about 2:1, 1:1 or about 0.5:1 or about 0.1:1.

In an embodiment, there is provided a process wherein:
the weight ratio of the recombinant host cell producing the SHC/HAC enzyme variant to EEH or a mixture of isomers of homofarnesol comprising EEH in an EE:EZ isomer mixture is 0.4 and the concentration of EEH is 450 g/l.

In an embodiment, there is provided a process wherein:
the weight ratio of the recombinant host cell producing the SHC/HAC enzyme variant to EEH or a mixture of isomers of homofarnesol comprising EEH in an EE:EZ isomer mixture is 1 and the concentration of EEH is 250 g/l.

In an embodiment, there is provided a process wherein:
the weight ratio of the recombinant host cell producing the SHC/HAC enzyme variant to EEH or a mixture of isomers of homofarnesol comprising EEH in an EE:EZ isomer mixture is 1 and the concentration of EEH is 300 g/l.

In a further aspect there is provided a SHC/HAC enzyme variant as defined herein.

In a further aspect there is provided a (−)-Ambrox obtained by or obtainable by the process as defined herein, in a solid form in an amorphous or crystalline form.

In a further aspect there is provided the use of (−)-Ambrox as obtained herein as part of a fragrance or a cosmetic or a consumer product such as fabric care, toiletry, beauty care, a cleaning product, a detergent product, and/or a soap product.

In a further aspect there is provided a fragrance or a cosmetic or a consumer product comprising (−)-Ambrox as obtained herein.

In a further aspect there is provided a nucleic acid sequence encoding the SHC/HAC enzyme variant as defined herein.

In a further aspect there is provided a construct comprising the nucleic acid sequence as defined herein.

In a further aspect there is provided a recombinant host cell comprising the nucleic acid sequence or the construct as defined herein.

### BRIEF DESCRIPTION OF THE FIGURES

FIG. 1: EEH conversion with AacSHC and 215G2SHC P1, P2, and P3 variants. Reactions were run with 8 g/l EEH (AacSHC wt 4 g/l EEH only), cells to an OD_{650 nm} of 10 applying individually optimized reaction conditions (T, pH, SDS).

FIG. 2 shows the reaction products of Homofarnesol SHC-catalyzed cyclization using a mixture of E,E- and E,Z-Homofarnesol isomers.

FIG. 3: Substrate specificity and product selectivity with P1, P2, and P3 SHC variants. Reactions were run with 8 g/l EEH, cells to an OD650 nm of 10 applying individually optimized reaction conditions (T, pH, SDS).

FIG. 4: Homofarnesol cyclization with SHC variants. Reactions were run with 125 g/l EEH and 250 g/l cells, at T, pH and SDS (SDS:cells ratio) conditions defined as optimal for each of the variants.

FIG. 5: Homofarnesol bioconversions with SHC variants. Reactions were run with 125 g/l EEH and 250 g/l cells, at T, pH and SDS (SDS:cells ratio) conditions defined as optimal for each of the variants.

FIG. 6: Substrate specificity and product selectivity in Homofarnesol bioconversions with SHC enzyme variants. Reactions were run with 125 g/l E,E-Homofarnesol and 250 g/l cells, at T, pH and SDS (SDS:cells ratio) conditions defined as optimal for each of the SHC enzymes.

FIG. 7: Homofarnesol bioconversions with SHC variants. Reactions were run with 250 g/l EEH and 250 g/l cells, at T, pH and SDS (SDS:cells ratio) conditions defined as optimal for each of the variants.

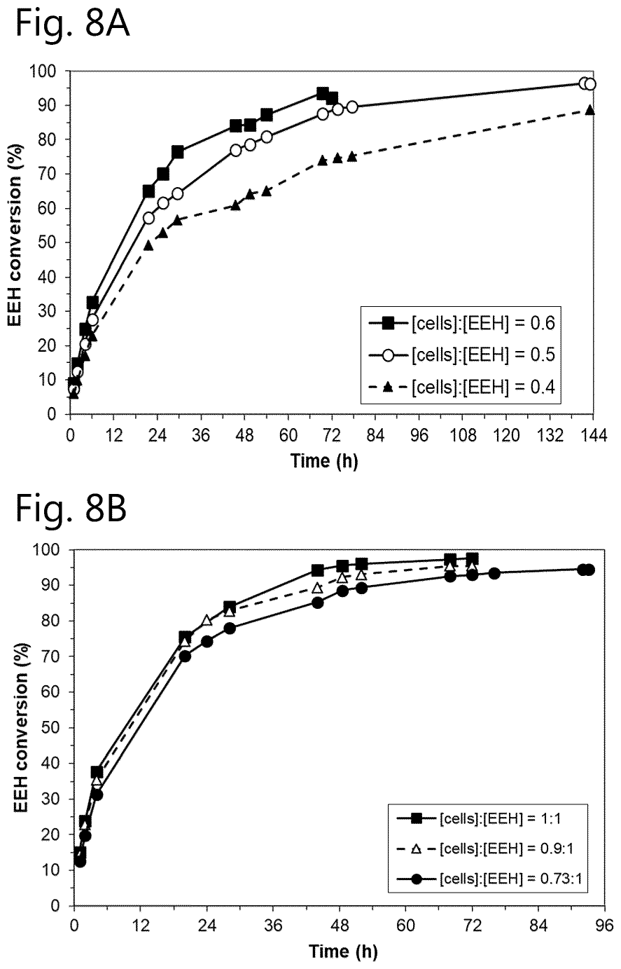

FIG. 8: Homofarnesol bioconversions with SHC variants. Reactions were run with A: 250 g/l EEH and SHC #65 P2, and with B: 300 g/l EEH and SHC #65, at varying [EEH]:[cells] ratios, and at T, pH and SDS (SDS:cells ratio) conditions defined as optimal for each of the variants.

FIG. 9: Squalene Hopene cyclase amino acid sequence alignment. The figure shows an amino acid sequence alignment of the squalene hopene cyclase enzymes listed in Table 3 and/or Table 16 prepared with CLUSTAL O (1.2.4). The amino acids at positions 169, 306, and 600 of the Alicyclobacillus acidocaldarius (AacSHC) sequence are highlighted in white on a black background. W at position 169 is almost strictly conserved as is G at position 600 throughout the amino acid sequences aligned: 15 out of 16 sequences, one single conservative substitution. A at position 306 is less conserved (9 out of 16 sequences).

FIG. 10: Bis-homofarnesol cyclization with SHC variants. Reactions were run with 4 g/l E,E-Bis-homofarnesol and cells to an OD_{650 nm} of 10, applying conditions individually defined as optimal for SHC #65 and SHC #65 P2 regarding T, pH and SDS concentration.

FIG. 11: Homofarnesol cyclization with wild-type and variantSHC enzymes. Reactions were run in deionized water at 30° C. with 2.36 g/l Homofarnesol and cells to an OD_{650 nm} of 6.

FIG. 12: Homofarnesol bioconversion with SHC #65 P2. Reactions were run with 400 g/l EEH and cells that had produced SHC #65 P2 at [cells]:[EEH] ratios of 0.6, 0.5, 0.4, and 0.3. Reactions were run at 35° C., pH 5.8, and at a constant [SDS]:[cells] ratio of 0.025.

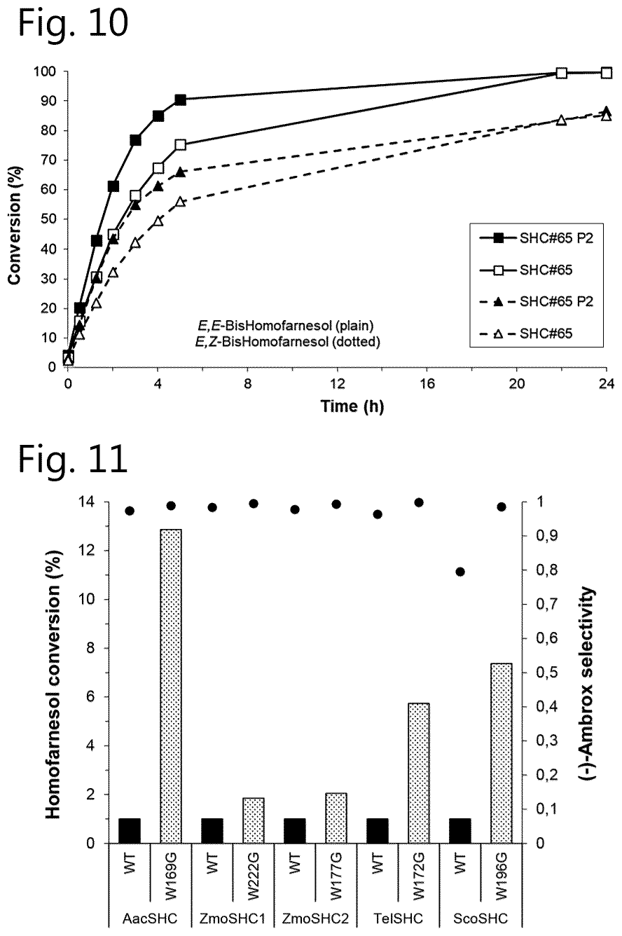

FIG. 13: Homofarnesol bioconversion with SHC #65 P2. Reactions were run with 450 g/l EEH and 180 g/l cells ([cells]:[EEH] ratio of 0.4), or 250 g/l EEH and 250 g/l cells ([cells]:[EEH] ratio of 1.0) at 35° C., pH 5.8, and a constant [SDS]:[cells] ratio of 0.030.

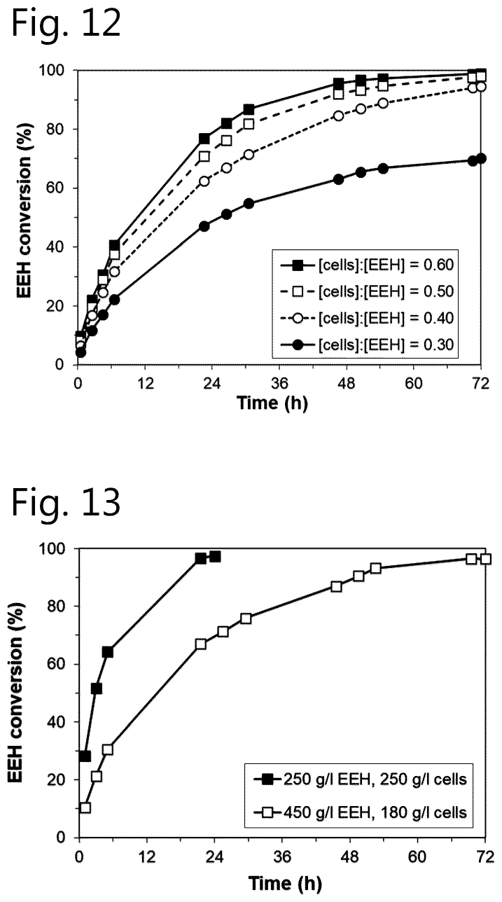

### SUMMARY OF THE SEQUENCES

SEQ ID NO: 1 is the wild-type Alicyclobacillus acidocaldarius (Aac) SHC amino acid sequence.

SEQ ID NO: 2 corresponds to SEQ ID NO: 1 with the substitutions M132R, A224V, 1432T, A557T and R613S and may be referred to as SHC/HAC enzyme variant #65 or SHC #65 variant herein.

SEQ ID NO: 3 may be referred to as 215G2 SHC (or 215G2 SHC variant) and corresponds to the wild-type AacSHC amino acid sequence with the mutations M132R, A224V and 1432T.

SEQ ID NO: 4 corresponds to SEQ ID NO: 1 with the substitution W169G (also called the Aac SHC P1).

SEQ ID NO:5 corresponds to SEQ ID NO: 1 with the substitutions W169G and G600M (also called the Aac SHC P2).

SEQ ID NO:6 corresponds to SEQ ID NO: 1 with the substitutions W169G, A306V and G600M (also called the Aac SHC P3).

SEQ ID NO: 7 corresponds to SEQ ID NO: 2 with the substitution W169G (also called the SHC #65 P1 variant).

SEQ ID NO:8 corresponds to SEQ ID NO: 2 with the substitutions W169G and G600M (also called the SHC #65 P2 variant).

SEQ ID NO:9 corresponds to SEQ ID NO: 2 with the substitutions W169G, A306V and G600M (also called the SHC #65 P3 variant).

SEQ ID NO: 10 corresponds to SEQ ID NO: 3 with the substitution W169G (also called the 215G2 SHC P1 variant).

SEQ ID NO:11 corresponds to SEQ ID NO: 3 with the substitutions W169G and G600M (also called the 215G2 SHC P2 variant).

SEQ ID NO:12 corresponds to SEQ ID NO: 3 with the substitutions W169G, A306V and G600M (also called the 215G2 SHC P3 variant).

SEQ ID NO: 13 to SEQ ID NO:34, SEQ ID NO:384 Wild type SHCs enzymes that may be used in the invention as SEQ ID NO:1 (see also tables 3 and 16). SEQ ID NO:13 is identical with SEQ ID NO: 384.

SEQ ID NO: 35 is the nucleotide sequence encoding the wild-type AacSHC.

SEQ ID NO: 36 is the nucleotide sequence encoding the polypeptide of SEQ ID NO: 2 (SHC #65 variant).

SEQ ID NO: 37 is the nucleotide sequence encoding the 215G2 variant (SEQ ID NO:3).

SEQ ID NO: 38 is the nucleotide sequence encoding the AacSHC P1 represented by SEQ ID NO:4.

SEQ ID NO: 39 is the nucleotide sequence encoding the AacSHC P2 represented by SEQ ID NO:5.

SEQ ID NO: 40 is the nucleotide sequence encoding the AacSHC P3 represented by SEQ ID NO:6.

SEQ ID NO: 41 is the nucleotide sequence encoding the SHC #65 P1 variant represented by SEQ ID NO:7.

SEQ ID NO: 42 is the nucleotide sequence encoding the SHC #65 P2 variant represented by SEQ ID NO:8.

SEQ ID NO: 43 is the nucleotide sequence encoding the SHC #65 P3 variant represented by SEQ ID NO:9.

SEQ ID NO: 44 is the nucleotide sequence encoding the 215G2 P1 variant represented by SEQ ID NO:10 SEQ ID NO: 45 is the nucleotide sequence encoding the 215G2 P2 variant represented by SEQ ID NO:11 SEQ ID NO: 46 is the nucleotide sequence encoding the 215G2 P3 variant represented by SEQ ID NO:12 SEQ ID NO: 47 corresponds to SEQ ID NO: 1 with the substitutions M132R, A224V, 1432T, Y81H, A557T and R613S and may be referred to as SHC/HAC enzyme variant #66 herein.

SEQ ID NO: 48 corresponds to SEQ ID NO: 1 with the substitutions M132R, A224V, 1432T, Y81H, H431L and A557T and may be referred to as SHC/HAC enzyme variant #110B8 herein.

SEQ ID NO: 49 corresponds to SEQ ID NO: 1 with the substitutions M132R, A224V, 1432T, T90A and R613S and may be referred to as SHC/HAC enzyme variant #90C7 herein.

SEQ ID NO: 50 corresponds to SEQ ID NO: 1 with the substitutions M132R, A224V, 1432T, A172T and M277K and may be referred to as SHC/HAC enzyme variant #115A7 herein.

SEQ ID NO: 51 is the amino acid sequence of the SHC/HAC variant 215G2 SHC (represented by SEQ ID NO:3) with the additional mutation L37Q.

SEQ ID NO: 52 is the amino acid sequence of the SHC/HAC variant 215G2 SHC (represented by SEQ ID NO:3) with the additional mutation V1741.

SEQ ID NO: 53 is the amino acid sequence of the SHC/HAC variant 215G2 SHC (represented by SEQ ID NO:3) with the additional mutations V1741 and F601Y.

SEQ ID NO: 54 is the amino acid sequence of the SHC/HAC variant 215G2 SHC (represented by SEQ ID NO:3) with the additional mutations L37Q, V1741 and F601Y.

SEQ ID NO: 55-296, 307-310 are wild type amino acid sequence of SHC/HAC as identified in table 2 or 16.

SEQ ID NO: 297-300 are nucleotide sequences encoding SEQ ID NO:47-50.

SEQ ID NO: 301 is the amino acid sequence of an GmoSHC variant with V45L, Q54E, M1841, T326S, F624Y.

SEQ ID NO: 302 is the amino acid sequence of TeISHC with W172G, P311V, F425Y, G609A mutations.

SEQ ID NO: 303 is the amino acid sequence of ZmoSHC1 with Q221S, W222G, A368V, F486Y, G667A mutations.

SEQ ID NO: 304 is the amino acid sequence of TeISHC with W172G mutation (P1).

SEQ ID NO: 305 is the amino acid sequence of AacSHC with W169G, G600M, M132R, A224V and 1432T mutations.

SEQ ID NO: 306 is the amino acid sequence of AacSHC with W169G, G600M, M132R and 1432T mutations.

SEQ ID NO: 307-310 (together with SEQ ID NO: 55-296) are wild type amino acid sequence of SHC/HAC as identified in table 2 or 16.

SEQ ID NO: 311 is the amino acid sequence of ScoSHC1 with W196G mutation (P1)

SEQ ID NO: 312 is the amino acid sequence of ZmoSHC1 with W222G mutation (P1)

SEQ ID NO: 313 is the amino acid sequence of ZmoSHC2 with W177G mutation (P1)

SEQ ID NO:314 is the motif DXDDTA found in SHC/HAC.

SEQ ID NO: 315-360 are amino acid sequences of SHC variants, especially SEQ ID NO: 315-347, 348, 350-352 (except 349) are SHC variants derived from the AaC SHC (SEQ ID NO:1).

SEQ ID No. 315 corresponds to SEQ ID NO: 1 with the substitution W169G.

SEQ ID No. 316 corresponds to SEQ ID NO: 1 with the substitution W169A.

SEQ ID No. 317 corresponds to SEQ ID NO: 1 with the substitution W169V.

SEQ ID No. 318 corresponds to SEQ ID NO: 1 with the substitutions W169G and G600A.

SEQ ID No. 319 corresponds to SEQ ID NO: 1 with the substitutions W169G and G600V.

SEQ ID No. 320 corresponds to SEQ ID NO: 1 with the substitutions W169G and G600L.

SEQ ID No. 321 corresponds to SEQ ID NO: 1 with the substitutions W169G and G6001.

SEQ ID No. 322 corresponds to SEQ ID NO: 1 with the substitutions W169G and G600M.

SEQ ID No. 323 corresponds to SEQ ID NO: 1 with the substitutions W169A and G600A.

SEQ ID No. 324 corresponds to SEQ ID NO: 1 with the substitutions W169A and G600V.

SEQ ID No. 325 corresponds to SEQ ID NO: 1 with the substitutions W169A and G600L.

SEQ ID No. 326 corresponds to SEQ ID NO: 1 with the substitutions W169A and G6001.

SEQ ID No. 327 corresponds to SEQ ID NO: 1 with the substitutions W169A and G600M.

SEQ ID No. 328 corresponds to SEQ ID NO: 1 with the substitutions W169V and G600A.

SEQ ID No. 329 corresponds to SEQ ID NO: 1 with the substitutions W169V and G600V.

SEQ ID No. 330 corresponds to SEQ ID NO: 1 with the substitutions W169V and G600L.

SEQ ID No. 331 corresponds to SEQ ID NO: 1 with the substitutions W169V and G6001.

SEQ ID No. 332 corresponds to SEQ ID NO: 1 with the substitutions W169V and G600M.

SEQ ID No. 333 corresponds to SEQ ID NO: 1 with the substitutions W169G, G600A and A306V.

SEQ ID No. 334 corresponds to SEQ ID NO: 1 with the substitutions W169G, G600V and A306V.

SEQ ID No. 335 corresponds to SEQ ID NO: 1 with the substitutions W169G, G600L and A306V.

SEQ ID No. 336 corresponds to SEQ ID NO: 1 with the substitutions W169G, G6001 and A306V.

SEQ ID No. 337 corresponds to SEQ ID NO: 1 with the substitutions W169G, G600M and A306V.

SEQ ID No. 338 corresponds to SEQ ID NO: 1 with the substitutions W169A, G600A and A306V.

SEQ ID No. 339 corresponds to SEQ ID NO: 1 with the substitutions W169A, G600V and A306V.

SEQ ID No. 340 corresponds to SEQ ID NO: 1 with the substitutions W169A, G600L and A306V.

SEQ ID No. 341 corresponds to SEQ ID NO: 1 with the substitutions W169A, G6001 and A306V.

SEQ ID No. 342 corresponds to SEQ ID NO: 1 with the substitutions W169A, G600M and A306V.

SEQ ID No. 343 corresponds to SEQ ID NO: 1 with the substitutions W169V, G600A and A306V.

SEQ ID No. 344 corresponds to SEQ ID NO: 1 with the substitutions W169V, G600V and A306V.

SEQ ID No. 345 corresponds to SEQ ID NO: 1 with the substitutions W169V, G600L and A306V.

SEQ ID No. 346 corresponds to SEQ ID NO: 1 with the substitutions W169V, G6001 and A306V.

SEQ ID No. 347 corresponds to SEQ ID NO: 1 with the substitutions W169V, G600M and A306V.

SEQ ID No. 348 corresponds to SEQ ID NO: 1 with the substitutions M132R, A224V and 1432T.

SEQ ID NO:349 is a nucleotide sequence coding for a SHC variant derived from AacSHC.

SEQ ID No. 350 corresponds to SEQ ID NO: 1 with the substitutions W169G and A306V.

SEQ ID No. 351 corresponds to SEQ ID NO: 1 with the substitutions W169A and A306V.

SEQ ID No. 352 corresponds to SEQ ID NO: 1 with the substitutions W169V and A306V.

SEQ ID NO:353 corresponds to SEQ ID NO: 13 with the substitutions W222G and G667M (also called the ZmoSHC1 P2 variant).

SEQ ID NO:354 corresponds to SEQ ID NO: 13 with the substitutions W222G, A368V and G667M (also called the ZmoSHC1 P3 variant).

SEQ ID NO:355 corresponds to SEQ ID NO: 15 with the substitutions W177G and G619M (also called the ZmoSHC2 P2 variant).

SEQ ID NO:356 corresponds to SEQ ID NO: 15 with the substitutions W177G, A321V and G619M (also called the ZmoSHC2 P3 variant).

SEQ ID NO:357 corresponds to SEQ ID NO: 32 with the substitutions W196G and G629M (also called the ScoSHC P2 variant).

SEQ ID NO:358 corresponds to SEQ ID NO: 32 with the substitutions W196G, A335V and G629M (also called the ScoSHC P3 variant).

SEQ ID NO:359 corresponds to SEQ ID NO: 23 with the substitutions W172G and G609M (also called the TeISHC P2 variant).

SEQ ID NO:360 corresponds to SEQ ID NO: 23 with the substitutions W172G, A311V and G609M (also called the TeISHC P3 variant).

SEQ ID NO: 361-383 are amino acid sequences of SHC variants derived from Aac SHC (SEQ ID NO:1) as identified in Table 18.

SEQ ID No. 361 corresponds to SEQ ID NO: 1 with the substitution T77A.

SEQ ID No. 362 corresponds to SEQ ID NO: 1 with the substitution 192V.

SEQ ID No. 363 corresponds to SEQ ID NO: 1 with the substitution F129L.

SEQ ID No. 364 corresponds to SEQ ID NO: 1 with the substitution M132R.

SEQ ID No. 365 corresponds to SEQ ID NO: 1 with the substitution A224V.

SEQ ID No. 366 corresponds to SEQ ID NO: 1 with the substitution 1432T.

SEQ ID No. 367 corresponds to SEQ ID NO: 1 with the substitution Q579H.

SEQ ID No. 368 corresponds to SEQ ID NO: 1 with the substitution F601Y.

SEQ ID No. 369 corresponds to SEQ ID NO: 1 with the substitutions M132R and 1432T.

SEQ ID No. 370 corresponds to SEQ ID NO: 1 with the substitution F601Y.

SEQ ID No. 371 corresponds to SEQ ID NO: 1 with the substitutions T77A, 192V and F129L.

SEQ ID No. 372 corresponds to SEQ ID NO: 1 with the substitutions Q579H and F601Y.

SEQ ID No. 373 corresponds to SEQ ID NO: 1 with the substitutions F129L.

SEQ ID No. 374 corresponds to SEQ ID NO: 1 with the substitutions F129L and F601Y.

SEQ ID No. 375 corresponds to SEQ ID NO: 1 with the substitutions F129L, M132R and 1432T.

SEQ ID No. 376 corresponds to SEQ ID NO: 1 with the substitutions M132R, 1432T and F601Y.

SEQ ID No. 377 corresponds to SEQ ID NO: 1 with the substitutions F129L, M132R, 1432T, and F601Y.

SEQ ID No. 378 corresponds to SEQ ID NO: 1 with the substitution F605W.

SEQ ID No. 379 corresponds to SEQ ID NO: 1 with the substitutions M132R, A224V, 1432T, A557T and H431L, and may be referred to as SHC/HAC enzyme variant #49 or SHC #49 variant herein.

SEQ ID No. 380 corresponds to SEQ ID NO: 1 with the substitutions M132R, A224V, 1432T, Y81H, A557T and R613S and may be referred to as SHC/HAC enzyme variant #66 or SHC #66 variant herein.

SEQ ID No. 381 corresponds to SEQ ID NO: 1 with the substitutions M132R, A224V, 1432T, Y81H, H431L and A557T, and may be referred to as SHC/HAC enzyme variant #110B8 or SHC #110B8 variant herein.

SEQ ID No. 382 corresponds to SEQ ID NO: 1 with the substitutions M132R, A224V, 1432T, T90A and R613S, and may be referred to as SHC/HAC enzyme variant #90C7 or SHC #90C7 variant herein.

SEQ ID No. 383 corresponds to SEQ ID NO: 1 with the substitutions M132R, A224V, 1432T, A172T and M277K, and may be referred to as SHC/HAC enzyme variant #115A7 or SHC #115A7 variant herein.

SEQ ID No. 385 corresponds to SEQ ID NO: 3 with the substitutions W169G and A306V.

SEQ ID No. 386 corresponds to SEQ ID NO: 2 with the substitutions W169G and A306V.

### DETAILED DESCRIPTION

### SHC/HAC Enzymes and Variants Thereof

As used herein, the term “SHC enzyme” means a wild-type (WT) Squalene Hopene Cyclase enzyme that is naturally occurring in, for example, a thermophilic bacterium such as Alicyclobacillus acidocaldarius (Aac). SHCs that act in the cyclisation of homofarnesol to Ambrox may also be referred to as Homofarnesol Ambrox Cyclase (HAC) enzymes. Therefore, the term “SHC/HAC enzyme” may be used herein.

As used herein, the term “variant” is to be understood as a polypeptide which differs in comparison to the polypeptide from which it is derived by one or more changes or alteration in the amino acid sequence. The polypeptide from which a variant is derived is also known as the parent or reference polypeptide. Typically, a variant is constructed artificially, preferably by gene-technological means. Typically, the polypeptide from which the variant is derived is a wild-type protein or wild-type protein domain. However, the variants usable in the present disclosure may also be derived from homologs, orthologs, or paralogs of the parent polypeptide or from artificially constructed variants, provided that the variant exhibits at least one biological activity of the parent polypeptide. The changes in the amino acid sequence may be amino acid exchanges (substitutions), insertions, deletions, N-terminal truncations, or C-terminal truncations, or any combination of these changes, which may occur at one or several sites.

As used herein, the term “SHC/HAC enzyme variant” means an enzyme that is derived from a wild-type SHC enzyme (such as Aac SHC enzyme, represented by SEQ ID NO:1) but has one or more amino acid alterations compared to the wild-type SHC enzyme and is therefore not naturally occurring in a prokaryote. Table 2 gives a list of wild-type SHC that may be used in the present invention to generate a SHC/HAC enzyme variant. Tables 3 and 16 give a list of preferred wild type SHC to be used in the present invention to generate a SHC/HAC enzyme variant. Table 3 lists the following SHC/AHC enzymes: AaC SHC represented by SEQ ID NO:1, ZmoSHC1 represented by SEQ ID NO:13 or 384, ZmoSCH2 represented by SEQ ID NO:15, BjpSHC represented by SEQ ID NO:16, Burkhoderia ambifaria SHC represented by SEQ ID NO:17 or 18, Bacillus anthracis SHC represented by SEQ ID NO:19, Frankia alni SHC represented by SEQ ID NO:20, Rhodopseudomonas palustris SHC represented by SEQ ID NO:21, GmoSCO SHC represented by SEQ ID NO:22, Tel SHC represented by SEQ ID NO:23, ApaSHC1 represented by SEQ ID NO:24, BmeSHC represented by SEQ ID NO:25, SaISHC represented by SEQ ID NO:26, ApaSHCA represented by SEQ ID NO:27, BamSHC1 represented by SEQ ID NO:28, BamSHC2 represented by SEQ ID NO: 29, PcaSHC2 (or Syntrophotalea carbinolica DSM 2380) represented by SEQ ID NO:30, RpaSHC1 represented by SEQ ID NO:31, ScoSHC represented by SEQ ID NO:32, SfuSHC represented by SEQ ID NO:33, TtuSHC represented by SEQ ID NO:34. More preferred wild type SHC to be used in the present invention to generate a SHC/HAC enzyme variant are: AaC SHC represented by SEQ ID NO:1, ZmoSHC1 represented by SEQ ID NO:13, ZmoSCH2 represented by SEQ ID NO:15, Tel SHC represented by SEQ ID NO:23 and ScoSHC represented by SEQ ID NO:32.

The one or more amino acid alterations may, for example, modify (e.g. increase) the enzymatic activity for a substrate (e.g. EEH). In the context of the invention, a SHC/HAC enzyme variant may be derived from a SHC/HAC variant. Examples of SHC/HAC variants include SEQ ID NO:2, 3, 4748, 49, 50, 51, 52, 53, 54, 361, 362, 363, 364, 365, 366, 367, 368, 369, 370, 371, 372, 373, 374, 375, 376, 377, 378, 379, 380, 381, 382 or 383.

Assays for determining and quantifying SHC/HAC enzyme and/or SHC/HAC enzyme variant activity are described herein and are known in the art. By way of example, SHC/HAC enzyme and/or SHC/HAC enzyme variant activity can be determined by incubating purified SHC/HAC enzyme or enzyme variant or extracts from host cells or a complete recombinant host organism that has produced the SHC/HAC enzyme or enzyme variant with an appropriate substrate under appropriate conditions and carrying out an analysis of the reaction products (e.g. by gas chromatography (GC) or HPLC analysis). Further details on SHC/HAC enzyme and/or SHC/HAC enzyme variant activity assays and analysis of the reaction products are provided in the Examples. These assays include producing the SHC/HAC enzyme variant in recombinant host cells (e.g. E. coli).

As used herein, the term “activity” means the ability of an enzyme to react with a substrate to provide a desired product. The activity can be determined in what is known as an activity test for monitoring the formation of the desired product. The SHC/HAC enzyme derivatives of the present disclosure may be characterized by their ability to cyclize homofamesol (e.g. EEH) into (−)-Ambrox and demonstrate a biological activity such as an HAC activity. The SHC/HAC enzyme derivatives of the present disclosure may be characterized by their ability to cyclize bishomofamesol (e.g. E,E-Bishomofarnesol) into Ambra oxide.

In the context of the application, an activity or a biological activity of a SHC/HAC variant is compared with the corresponding activity or biological activity of the wild type, parent, reference SHC/HAC it derives from and under the same conditions. Examples of wild type SHC/HAC enzymes have been identified in table 2, 3 or 16. In an embodiment, wild type SHC/HAC enzymes are those represented by SEQ ID NO:1 or by any of SEQ ID NO:13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310.

Examples of parent or reference SHC/HAC enzyme used to create the SHC/HAC variant of the invention may be represented by any of SEQ ID NO: 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383.

A “biological activity” as used herein, refers to any activity a polypeptide may exhibit, including without limitation: enzymatic activity; substrate specificity, selectivity (such as substrate selectivity and/or product selectivity), rate of conversion (such as an EEH:EZH conversion ratio), yield, binding activity to another compound (e.g. binding to another polypeptide, in particular binding to a receptor, or binding to a nucleic acid); inhibitory activity (e.g. enzyme inhibitory activity); activating activity (e.g. enzyme-activating activity); or toxic effects. It is not required that the variant exhibits such an activity to the same extent as the parent or wild-type or reference polypeptide. A variant is regarded as a variant within the context of the present application, if it exhibits the relevant activity to a degree of at least 10%, at least 20%, at least 30%, at least 40%, at least 50%, at least 60%, at least 70%, at least 80%, at least 90%, at least 100%, at least 110%, at least 120%, at least 130%, at least 140%, at least 150% or at least 200% of the activity of the parent polypeptide. Likewise, a variant is regarded as a variant within the context of the present application, if it exhibits the relevant biological activity to a degree of at least 10%, at least 20%, at least 30%, at least 40%, at least 50%, at least 60%, at least 70%, at least 80%, at least 90%, at least 100%, at least 110%, at least 120%, at least 130%, at least 140%, at least 150% or at least 200% of the activity of the parent polypeptide (as the terms derivative and variant are used interchangeably throughout the present disclosure).

In an embodiment, the SHC/HAC enzyme variants described herein show a better yield (i.e. increased yield) compared to the yield obtained using a reference SHC enzyme (e.g. a wild-type SHC/HAC enzyme such as AacSHC/AHC enzyme or a known SHC/HAC enzyme variant or the parent SHC the variant derives from). The term “yield” refers to the gram of recoverable product (i.e. (−)-Ambrox or Ambra oxide) per gram of feedstock (which can be calculated as a percent molar conversion rate). In this context, “increase” may mean an increase of at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200% or at least 400% of the yield obtained using a reference SHC enzyme.

In an embodiment, “selectivity” refers to “product selectivity” and describes the ability to produce a particular compound (eg (−)-Ambrox or Ambra oxide) in an enzymatically catalyzed method as described in a substantially enriched/predominant form (“product selectivity”) from a mixture of several substrate isomers. In an embodiment, the mixture of homofarnesol substrate isomers is selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)]. In an embodiment, the mixture is [(3E,7E), (3Z,7E)] also designated [EE:EZ] or EEH:EZH. In an embodiment, the ratio of EEH:EZH within this mixture may range from about 50:50 to about 99:01 or from about 60:40 to about 99:1 or from about 70:30 to about 95:5 or from about 80:20 to about 95:5.

In a preferred embodiment, “selectivity” refers to “product selectivity” and describes the ability to produce a particular compound (eg (−)-Ambrox, i.e. compound (I)) in an enzymatically catalyzed method as described in a substantially enriched/predominant form (“product selectivity”) from a EEH:EZH mixture with a ratio of 80:20 or 90:10.

Within this context, “substantially enriched/predominant form” may mean that the total products formed as a result of the cyclisation reaction of the enzyme variant of WT SHC may, for example, consist essentially of or consist of:
compound of formula (I) ((−)-Ambrox) described herein when using any of the homofarnesol substrate isomer mixture identified above (preferably EEH:EZH, more preferably with a ratio of 80:20 or 90:10) or
compounds of formula (I) ((−)-Ambrox) and formula (III) described herein when using any of the homofarnesol substrate isomer mixture identified above (preferably EEH:EZH, more preferably with a ratio of 80:20 or 90:10).

It means that compounds of formula (II) and (IV) may not detectable at the end of the process or during the first 3, 4, 5 or 6 or 12 or 18 hours of the process. The detection of compounds of formula (I), (II), (III) and (IV) may be done using techniques known to the skilled person, preferably those used in the experimental part. It also means that in the absence of any downstream processing steps, only the compound of formula (I) or only the compounds of formula (I) and (III) are produced. This means that down stream processing (DSP) may be simplified because both the degree of substrate conversion and the conversion rate of the substrate has been increased. In other words, the DSP is simplified since the amount of unreacted homofarnesol present at the end of the reaction may be reduced. It is known that inreacted homofarnesol dissolves at least part of the solid (−)-Ambrox formed and that solid (−)-Ambrox formed may also dissolve in the other liquid by-products which, if not present (eg compounds II and/or IV) may simplify the DSP steps.

In this context, “a downstream processing step” means a separation of solid (−)-Ambrox from unreacted homofarnesol substrate as well as a separation of (−)-Ambrox from other by-products (eg compounds II and/or Ill and/or IV). DSP steps may include but are not limited to one or more of a centrifugation, a filtration, a steam or organic solvent extraction or distillation, or a selective crystallization step. In this context an example of a simplified DSP is where a distillation step may be more efficient at separating (−)-Ambrox from unreacted homofarnesol than separating (−)-Ambrox from the by-products produced. Each of these downstream processing steps have been later defined herein. Exemplary downstream processing steps are provided in WO2022/023464 the contents of which are incorporated herein by reference.

In an embodiment, the SHC/HAC enzyme variants described herein show an increased product selectivity (i.e increased selectivity for (−)-Ambrox or Ambra oxide) compared to the product selectivity obtained using a reference SHC enzyme (e.g. a wild-type SHC/HAC enzyme such as AacSHC/AHC enzyme represented by SEQ ID NO:1 or of any other wild type SHC/HAC enzyme represented by SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or a known SHC/HAC enzyme variant or the parent SHC the variant derives from, for example any SHC/HAC represented by SEQ ID NO: 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383). The term “product selectivity” also refers to the gram of recoverable product (i.e. (−)-Ambrox or Ambra oxide) per gram of total products formed (which can be calculated as a percent molar conversion rate). In this context, “increase” may mean an increase of at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200% or at least 400% of the yield obtained using a reference SHC enzyme.

In an embodiment, “selectivity” means an ability to preferentially convert a particular substrate isomer (eg the EEH isomer) into a specific product in an enzymatically catalyzed method as described herein out of a plurality/mixture of several substrate isomers (“substrate selectivity”).

More specifically, this means that a particular product (−)-Ambrox is enriched with respect to the enzymatic conversion of a specific substrate (EEH) isomer from a mixture of several substrate isomers.

Therefore, “substrate selectivity” of an enzyme or enzyme variant refers to the ability of the enzyme or enzyme variant to react with a particular substrate isomer compared to another substrate isomer, while the enzyme is in contact with a mixture comprising at least two distinct substrate isomers. For example, a WT SHC enzyme (such as those represented by SEQ ID NO:1 or by any of the SHC/HAC disclosed in tables 2, 3 or 16 or represented by SEQ ID NO:13, 14, 15, 16, 17, 18, 19, 20, 21, 22, 23, 24, 25, 26, 27, 28, 29, 30, 31, 32, 33, 34, 55. 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or enzyme variant of WT SHC (such as those represented by any of SEQ ID NO: 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383) that is selective for EEH over other isomers of homofarnesol or selective for BisEEH over other isomers of bishomofarnesol means that the WT SHC enzyme or enzyme variant of WT SHC is more likely to convert EEH than other isomers of homofarnesol or to convert BisEEH than other isomers of bishomofarnesol. In an embodiment, the mixture of homofarnesol substrate isomers comprising at least two distinct substrate isomers of homofarnesol comprises the EEH isomer and is selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)]. In an embodiment, the mixture is [(3E,7E), (3Z,7E)] also designated [EE:EZ] or EEH:EZH. In an embodiment, the ratio of EEH:EZH within this mixture may range from about 55:45, 50:50 to about 99:01 or from about 60:40 to about 99:1 or from about 70:30 to about 95:5 or from about 80:20 to about 95:5.

For example, the wt % of total products formed as a result of the reaction of the enzyme variant of a WT or of a reference or parent SHC with the mixture comprising at least two distinct substrate isomers of homofarnesol, said mixture comprising EEH may be at least about 1 percentage point greater than the wt % of total products formed as a result of the reaction of the corresponding WT or parent or reference of SHC enzyme with the same mixture comprising EEH. For example, the wt % of total products formed as a result of the reaction of said enzyme variant may be at least about 2 or at least about 3 or at least about 4 percentage points greater than the wt % of total products formed as a result of the reaction of WT or reference or parent SHC with the same mixture comprising EEH. For example, the wt % of total products formed as a result of the reaction of said enzyme variant may be up to about 40 or up to about 30 or up to about 20 or up to about 15 or up to about 10 percentage points greater than the wt % of total products formed as a result of the reaction of WT or reference or parent SHC with the same mixture comprising EEH. For example, the wt % of total products formed as a result of the reaction of said enzyme variant with a mixture comprising EEH may be from about 1 to about 40 or from about 2 to about 30 or from about 3 to about 20 or from about 4 to about 10 percentage points greater than the wt % of total products formed as a result of the reaction of WT or reference or parent SHC with the same mixture comprising EEH. The total products formed as a result of the reaction of the enzyme variant of a WT SHC or of reference or parent SHC may, for example, comprise, consist essentially of or consist of compounds of formula (II) ((−)-Ambrox) and formula (IV) described herein when a mixture comprising at least two distinct substrate isomers of homofarnesol, said mixture comprising EEH is used as a substrate. In all these embodiments, the mixture of homofarnesol substrate isomers comprising at least two distinct substrate isomers of homofarnesol comprises the EEH isomer and is selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)]. In an embodiment, the mixture is [(3E,7E), (3Z,7E)] also designated [EE:EZ] or EEH:EZH. In an embodiment, the ratio of EEH:EZH within this mixture may range from about 55:45, 50:50 to about 99:01 or from about 60:40 to about 99:1 or from about 70:30 to about 95:5 or from about 80:20 to about 95:5.

The total products formed as a result of the reaction of the enzyme variant of WT or of a reference or parent SHC may, for example, comprise, consist essentially of or consist of compounds of formula (X) and/or formula (XII) described herein when bisEEH is used as a substrate.

For example, the wt % of (−)-Ambrox formed using the enzyme variant of a WT SHC/HAC (such as those WT SHC/HAC represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or of a reference/variant SHC/HAC (such as those represented by SEQ ID NO: 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383) may, for example, be at least about 1 percentage point greater than the wt % of (−)-Ambrox formed as a result of the reaction of the WT or reference/variant SHC with a mixture comprising at least two distinct substrate isomers of homofarnesol, said mixture comprising EEH. For example, the wt % of (−)-Ambrox formed as a result of the reaction of the enzyme variant of said WT or reference or patent SHC with said mixture comprising EEH may be at least about 2 or at least about 3 or at least about 4 percentage points greater than the wt % of (−)-Ambrox formed as a result of the reaction of said WT or reference or parent SHC with said mixture comprising EEH. For example, the wt % of (−)-Ambrox formed as a result of the reaction of said enzyme variant of WT SHC with said mixture comprising EEH may be up to about 40 or up to about 30 or up to about 20 or up to about 15 or up to about 10 percentage points greater than the wt % of (−)-Ambrox formed as a result of the reaction of said WT or said reference or parent SHC with said mixture comprising EEH. For example, the wt % of (−)-Ambrox formed as a result of the reaction of said enzyme variant of said WT or said parent or reference SHC with said mixture comprising EEH may be from about 1 to about 40 or from about 2 to about 30 or from about 3 to about 20 or from about 4 to about 10 percentage points greater than the wt % of (−)-Ambrox formed as a result of the reaction of said WT of said reference or parent SHC with said mixture comprising EEH. In all these embodiments, the mixture of homofarnesol substrate isomers comprising at least two distinct substrate isomers of homofarnesol comprises the EEH isomer and is selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)]. In an embodiment, the mixture is [(3E,7E), (3Z,7E)] also designated [EE:EZ] or EEH:EZH. In an embodiment, the ratio of EEH:EZH within this mixture may range from about 55:45, 50:50 to about 99:01 or from about 60:40 to about 99:1 or from about 70:30 to about 95:5 or from about 80:20 to about 95:5.

Selectivity of an enzyme variant of a WT SHC/HAC (such those represented by SEQ ID NO:1, or 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or of those parent or reference SHC/HAC (such as those represented by SEQ ID NO: 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383) may also be compared to the selectivity of the corresponding WT SHC/HAC or corresponding reference/parent SHC/HAC enzyme by comparing the EEH:EZH conversion ratio (i.e. % conversion of EEH:% conversion of EZH) or bisEEH:bisEZH conversion ratio (i.e. % conversion of bisEEH:% conversion of bisEZH) of reactions using each enzyme. This may be determined by measuring the amount of EEH and EZH or bisEEH and bisEZH remaining in the reaction mixture when the reaction has completed. Usually the reaction has completed after 18 hours or after 20 hours. Alternatively, the selectivity of an enzyme variant of a WT SHC/HAC (such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or of a reference or variant SHC/HAC (such as those represented by SEQ ID NO: 2, 3 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383) may also be compared to the selectivity of said corresponding WT or reference or parent SHC/HAC by comparing the ratio of the products arising from the conversion of EEH (compounds of formulae I and IV) and EZH (compounds of formulae II and Ill) respectively or the conversion of bisEEH (compounds of formula X and XII) and bisEZH (XI and XIII) respectively.

The SHC/HAC enzyme variant of a WT or of a reference or parent SHC/HAC may, for example, provide an EEH:EZH conversion ratio of at least about 2.0 in a process for making (−)-Ambrox from a mixture of homofarnesol comprising at least two distinct substrate isomers of homofarnesol, said mixture comprising EEH and EZH. In an embodiment, the mixture of homofarnesol substrate isomers comprising at least two distinct substrate isomers of homofarnesol comprises the EEH and the EZH isomers and is selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)]. In an embodiment, the mixture comprises EEH and EZH.

In an embodiment, the mixture is [(3E,7E), (3Z,7E)] also designated [EE:EZ] or EEH:EZH. In an embodiment, the ratio of EEH:EZH within this mixture may range from about 55:45, 50:50 to about 99:01 or from about 60:40 to about 99:1 or from about 70:30 to about 95:5 or from about 80:20 to about 95:5.

For example, the enzyme variant of a WT or of a reference or parent SHC/HAC may provide an EEH:EZH conversion ratio of at least about 2.5 or at least about 3.0 or at least about 3.5 in a process for making (−)-Ambrox from a mixture as defined above. In an embodiment, the mixture comprises EEH and EZH. For example, the enzyme variant of said WT SHC/HAC or of said reference or parent SHC/HAC may provide an EEH:EZH conversion ratio up to about 5.0 or up to about 4.5 or up to about 4.0 in a process for making (−)-Ambrox from the mixture as defined above. In an embodiment, said mixture comprises EEH and EZH. For example, the enzyme variant of said WT or of said reference or parent SHC/HAC may provide an EEH:EZH conversion ratio ranging from about 2.0 to about 5.0 or from about 2.5 to about 4.5 or from about 3.0 to about 4.0 in a process for making (−)-Ambrox from the mixture as defined above. In an embodiment, said mixture comprises EEH and EZH. This may, for example, be in contrast to the conversion ratio provided by AacSHC in a process for making (−)-Ambrox from a mixture comprising EEH and EZH, which may, for example, be less than about 2.0.

In particular, where the WT SHC enzyme or enzyme variant of WT SHC has a higher selectivity for EEH over other isomers of homofarnesol compared to WT AacSHC and/or variants of WT AacSHC, the wild-type SHC/HAC enzyme (e.g. from which the SHC/HAC enzyme variant may be derived) may be selected from TeISHC1, ApaSHC1, ZmoSHC1, ZmoSHC2, BjaSHC (or BjpSHC), GmoSHC BmeSHC, SaISHC, ApaSHCA. For example, where the WT SHC enzyme or enzyme variant of WT SHC has a higher selectivity for EEH over other isomers of homofarnesol compared to WT AacSHC and/or variants of WT AacSHC, the wild-type SHC/HAC enzyme (e.g. from which the SHC/HAC enzyme variant may be derived) may be selected from ZmoSHC1, BjaSHC (BjpSHC), GmoSHC, ApaSHC1 and BmeSHC.

In an embodiment, the SHC/HAC enzyme variants described herein show an increased rate of EEH conversion (or increased rate of BisEEH conversion) compared to the rate of conversion of a reference SHC enzyme (e.g. a wild-type SHC/HAC enzyme such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290. 291, 292, 293, 294, 295, 296, 307, 308, 309 or 310) or a known SHC/HAC enzyme variant or the parent SHC the variant derives from (such as those represented by SEQ ID NO:2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383). The term “conversion rate” refers to the amount of converted substrate (i.e. EEH or BisEEH) per gram of biocatalyst and per unit of time (which can be calculated as a percent molar conversion rate). In this context, “increase” may mean an increase of at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200% or at least 400% of the conversion rate obtained using a reference SHC enzyme.

In an embodiment, the SHC/HAC enzyme variants described herein show an increased specificity for their substrate EEH (or BisEEH) compared to the specificity of a reference SHC enzyme (e.g. a wild-type SHC/HAC enzyme such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or a known SHC/HAC enzyme variant or the parent SHC the variant derives from such as those represented by SEQ ID NO:2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383) for the same substrate. The term “substrate specificity” also refers to the amount of a particular converted substrate isomer (i.e. EEH or BisEEH) per gram of substrate initially present at a certain point of time of an enzymatically catalysed reaction, or during an “interval” of said reaction. In particular, said selectivity may be observed during an “interval” corresponding 1 to 99%, 2 to 95%, 3 to 90%, 5 to 85%, 10 to 80%, 15 to 75%, 20 to 70%, 25 to 65%, 30 to 60, or 40 to 50% conversion of the initial amount of the substrate. In an embodiment, the “interval” may be from 1 to 8 hours or from 2 to 7 or from 3 to 6 hours. In an embodiment, the “interval is 6 hours. In this context, “increase” may mean an increase of at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200% or at least 400% of the substrate specificity obtained using a reference SHC enzyme.

In additional embodiments, the SHC/HAC enzyme variants described herein show a modified (e.g. increased) productivity relative to a reference SHC enzyme (e.g. wild-type AacSHC (SEQ ID NO:1) or 215G2 AacSHC (SEQ ID NO:3) or SHC #65 (SEQ ID NO:2) or a parent SHC enzyme the variant derives from such as any of the following wild type SHC/HAC identified in table 2 or 3 or 16 or represented by SEQ ID NO:13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or derived from a variant such as any of the following SEQ ID NO: 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383). The term “productivity” refers to the amount of recoverable product (i.e. (−)-Ambrox or Ambra oxide) in grams per liter of reaction capacity per hour of bioconversion time (i.e. time after the substrate was added). The term “productivity” also refers to the amount of recoverable product in grams per liter of reaction capacity per hour of bioconversion time (ie time after the substrate was added) per gram of biocatalyst used in the reaction. In this context, “increase” may mean an increase of at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 200% or at least 400% of the productivity obtained using a reference SHC enzyme.

In an embodiment, the SHC/HAC enzyme variant disclosed herein exhibits:
an increased substrate specificity for EEH (or for BisEEH) when homofarnesol or bis-homofarnesol substrates are used,
an increased product selectivity for (−)-Ambrox (or Ambra oxide) when homofarnesol or bis-homofarnesol substrates are used,
an increased specificity for a particular isomer of substrates when substrates other than homofarnesol or bis-homofarnesol are used (eg ethyl-homofarnesol, hydroxyfarnesylacetone and the like—see, for example WO2021/110858 and WO2021/209482 respectively)
an increased productivity and/or
an increased degree of conversion of EEH (or BisEEH) as well as an increased conversion rate of EEH (or BisEEH) over the first 3 to 6 hours (or over the first 4, 5, or 6 hours) of the reaction,
   compared to the reference SHC enzyme (e.g. wild-type SHC such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295 296, 307, 308, 309, 310 or 215G2 AacSHC or SHC #65 or a parent SHC enzyme the variant derives from such as those represented by SEQ ID NO:2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383).

In an embodiment, the SHC/HAC enzyme variant disclosed herein exhibits:
an increased substrate specificity for EEH (or for BisEEH) when homofarnesol or bis-homofarnesol substrates are used
an increased product selectivity for (−)-Ambrox (or Ambra oxide) when homofarnesol or bis-homofarnesol substrates are used
an increased specificity for a particular isomer of substrates when substrates other than homofarnesol or bis-homofarnesol are used (eg ethyl-homofarnesol, hydroxyfarnesylacetone and the like—see, for example WO2021/110858 and WO2021/209482 respectively)—an increased productivity and
an increased degree of conversion of EEH (or BisEEH) as well as an increased conversion over the first 3 to 6 hours (or over the first 4, 5 or 6 hours) of the reaction,
   compared to the reference SHC enzyme (e.g. wild-type AacSHC or 215G2 AacSHC or SHC #65 or a parent SHC enzyme the variant derives from).

In further embodiments, the SHC/HAC enzyme variants described herein show a modified (i.e. increased) yield compared with the yield of a reference SHC enzyme (e.g. wild-type AacSHC (SEQ ID NO: 1) or SHC #65 (SEQ ID NO:2), 215G2 AacSHC (SEQ ID NO: 3), ZmoSHC1 represented by SEQ ID NO:13, ZmoSCH2 represented by SEQ ID NO:15, BjpSHC represented by SEQ ID NO:16, Burkhoderia ambifaria SHC represented by SEQ ID NO:17 or 18, Bacillus anthracis SHC represented by SEQ ID NO:19, Frankia alni SHC represented by SEQ ID NO:20, Rhodopseudomonas palustris SHC represented by SEQ ID NO:21, GmoSCO SHC represented by SEQ ID NO:22, Tel SHC represented by SEQ ID NO:23, ApaSHC1 represented by SEQ ID NO:24, BmeSHC represented by SEQ ID NO:25, SaISHC represented by SEQ ID NO:26, ApaSHCA represented by SEQ ID NO:27, BamSHC1 represented by SEQ ID NO:28, BamSHC2 represented by SEQ ID NO: 29, PcaSHC2 (or Syntrophotalea carbinolica DSM 2380) represented by SEQ ID NO:30, RpaSHC1 represented by SEQ ID NO:31, ScoSHC represented by SEQ ID NO:32, SfuSHC represented by SEQ ID NO:33, TtuSHC represented by SEQ ID NO:34.

In a preferred embodiment, the SHC/HAC enzyme variants described herein show a modified (i.e. increased) yield compared with the yield of a reference AacSHC (SEQ ID NO: 1) or SHC #65 (SEQ ID NO:2), or 215G2 AacSHC (SEQ ID NO: 3) or ZmoSHC1 represented by SEQ ID NO:13 or ZmoSCH2 represented by SEQ ID NO:15 or Tel SHC represented by SEQ ID NO:23 or ScoSHC represented by SEQ ID NO:32.

In particular, the wild-type SHC/HAC enzyme (e.g. from which the SHC/HAC enzyme variant may be derived) may be the Alicyclobacillus acidocaldarius (Aac) SHC/HAC enzyme, the Zymomonas mobilis (Zmo) SHC/HAC enzyme, the Bradyrhizobium japonicum (Bjp/Bja) SHC/HAC enzyme, the Acetobacter pasteurianus (Apa) SHC/HAC enzyme, the Bacillus megaterium (Bme) SHC/HAC enzyme or the Gluconobacter morbifer (Gmo) SHC/HAC enzyme. In particular, the wild-type SHC/HAC enzyme (e.g. from which the SHC/HAC enzyme variant may be derived) may be the Alicyclobacillus acidocaldarius (Aac) SHC/HAC enzyme.

The term “target yield factor” refers to the ratio between the product concentration obtained and the concentration of the SHC/HAC variant enzyme (for example, purified SHC/HAC enzyme variant or an extract from the recombinant host cells producing the SHC/HAC enzyme variant) in the reaction medium. In various embodiments, the SHC/HAC enzyme variants disclosed herein show a modified (e.g. increased) fold increase in enzymatic activity (e.g. a modified/increased homofarnesol Ambrox cyclase (HAC) activity) relative to the activity of a reference SHC protein (e.g. SEQ ID No.1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383). This increase in activity may be at least by a factor of: 2, 3, 4, 6, 8, 10, 12, 14, 16, 18, 20, 25, 30, 35, 40, 45, 50, 55, 60, 65, 70, 75, 80, 85, 90, 95, and/or 100.

As used herein, the term “amino acid alteration” means an insertion of one or more amino acids between two amino acids, a deletion of one or more amino acids or a substitution (which may be conservative or non-conservative) of one or more amino acids with one or more different amino acids relative to the amino acid sequence of a reference amino acid sequence. Substitutions replace the amino acids of the reference sequence with the same number of amino acids in the variant sequence. Reference amino acid sequences may, for example, be a wild-type (WT) amino acid sequence (for example SEQ ID NO: 1 or any of SEQ ID NO:13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or may, for example, itself be a SHC/HAC enzyme variant sequence (for example the Aac variant—SEQ ID NO: 2 or 3 or 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383). The word “substitution” is synonymous with the word “replacement”.

The amino acid alterations can be easily identified by a comparison of the amino acid sequences of the SHC/HAC enzyme variant with the amino acid sequence of the reference amino acid sequence.

Conservative amino acid substitutions may be made, for instance, on the basis of similarity in polarity, charge, size, solubility, hydrophobicity, hydrophilicity, and/or the amphipathic nature of the amino acid residues involved. The 20 naturally occurring amino acids as outlined above can be grouped into the following six standard amino acid groups:
(1) hydrophobic: Met, Ala, Val, Leu, lie;
(2) neutral hydrophilic: Cys, Ser, Thr; Asn, Gln;
(3) acidic: Asp, Glu;
(4) basic: His, Lys, Arg;
(5) residues that influence chain orientation: Gly, Pro; and
(6) aromatic: Trp, Tyr, Phe.

Accordingly, as used herein, the term “conservative substitutions” means an exchange of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt alpha-helices. Some preferred conservative substitutions within the above six groups are exchanges within the following sub-groups: (i) Ala, Val, Leu and lie; (ii) Ser and Thr; (ii) Asn and Gln: (iv) Lys and Arg; and (v) Tyr and Phe. Given the known genetic code, and recombinant and synthetic DNA techniques, the skilled scientist readily can construct DNAs encoding the conservative amino acid variants.

In a preferred embodiment (as demonstrated in Example 11) a set of conserved amino acids in the amino acid sequence of a wild type SHC, in particular wild type AacSHC of SEQ ID No. 1, comprises L22, Q26, G30, W32, A44, L48, Q72, G76, W78, Y95, L98, G102, A113, 1117, G121, G122, F129, T130, L134, A135, G138, W142, P146, W169, A170, R171, F217, D222, R237, 1261, P263, P281, S309, P310, W312, D313, T314, A320, W339, Q344, G349, D350, W351, G361, G362, A364, F365, N369, Y372, P373, D374, D376, D377, W406, Q411, G415, A419, P433, D436, D442, P443, D447, V448, Q479, G483, W485, G487, R488, W489, G490, N492, Y495, G496, T497, L504, W522, Q527, G531, G532, W533, G534, E535, S539, Y540, G547, T552, T556, W558, A559, A565, L581, Q585, G589, W591, G600, F601, P602, F605, Y609, Y612, F616, P617, A620 and R623 by reference to the wild type AacSHC of SEQ ID No 1.

In a preferred embodiment (as demonstrated in Example 14) a set of conserved amino acids in the amino acid sequence for five wild type SHC, in particular wild type AacSHC of SEQ ID No. 1, wild type ZmoSHC1 of SEQ ID No. 13, wild type ZmoSHC2 of SEQ ID No. 15, wild type TeISHC of SEQ ID No. 23 and wild type ScoSHC of SEQ ID No. 32 is presented.

As used herein, “non-conservative substitutions” or “non-conservative amino acid exchanges” are defined as exchanges of an amino acid by another amino acid listed in a different group of the six standard amino acid groups (1) to (6) as shown above. Typically, the SHC/HAC enzyme variants described herein are prepared using non-conservative substitutions which alter the biological function (e.g. HAC activity) of the disclosed SHC/HAC enzyme variants. For ease of reference, the one-letter amino acid symbols recommended by the IUPAC-IUB Biochemical Nomenclature Commission are indicated as follows. The three letter codes are also provided for reference purposes.

TABLE 1
Amino acid nomenclature

| One Letter Code | Three Letter Code | Amino Acid Name |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Amino acid alterations such as amino acid substitutions may be introduced using known protocols of recombinant gene technology including PCR, gene cloning, site-directed mutagenesis of cDNA, transfection of host cells, and in vitro transcription which may be used to introduce such changes to the reference sequence resulting in an SHC/HAC enzyme variant. The enzyme variants can then be screened for SHC/HAC functional activity.

Suitable sources of SHC/HAC enzymes are identified in table 2, 3 or 16. In an embodiment, suitable sources include, for example, Alicyclobacillus acidocaldarius (Aac), Zymomonas mobilis (Zmo), Bradyrhizobium japonicum (Bjp), Gluconobacter morbifer (Gmo), Burkholderia ambifaria, Bacillus anthracis, Methylococcus capsulatus, Frankia alni, Acetobacter pasteurianus (Apa), Thermosynechococcus elongatus (Tel), Streptomyces coelicolor (Sco), Rhodopseudomonas palustris (Rpa), Teredinibacter turnerae (Ttu), Pelobacter carbinolicus (Pca) or Syntrophotalea carbinolica DSM 2380, Bacillus megaterium (Bme), Streptomyces albolongus (Sal) and Tetrahymena pyriformis (see, for example WO 2010/139719, US 2012/01345477, WO 2012/066059, the contents of which are incorporated herein by reference).

In particular, the SHC/HAC enzyme (e.g. from which the SHC/HAC enzyme variant may be derived) may be the Alicyclobacillus acidocaldarius (Aac) SHC/HAC enzyme, the Zymomonas mobilis SHC/HAC (ZmoSHC1) enzyme the Bradyrhizobium japonicum (Bjp or Bja) SHC/HAC enzyme or the Gluconobacter morbifer (Gmo) SHC/HAC enzyme or an Acetobacter pasteurianus SHC/HAC (ApaSHC1) enzyme or the Bacillus megaterium (Bme) SHC/HAC enzyme. In particular, the SHC/HAC enzyme (e.g. from which the SHC/HAC enzyme variant may be derived) may be the Alicyclobacillus acidocaldarius (Aac) SHC/HAC enzyme. This enzyme is represented by SEQ ID NO:1.

For ease of reference, the designation “AacSHC” may be used to refer to the Alicyclobacillus acidocaldarius (Aac) SHC/HAC enzyme, “ZmoSHC” may be used to refer to the Zymomonas mobiis (Zmo) SH19HAC enzymes, BjpSHC or BjaSHC may be used to refer to the Bradyrhizobium japonicum (Bjp) SHC/HAC enzyme, “ApaSHC” may be used to refer to the Acetobacter pasteurianus (Apa) SHHAC enzymes, “BmeSHC” may be used to refer to Bacillus megaterium (Bme) SH18HAC enzyme, “SaISH” may be used to refer to the Streptomyces albolongus (Sal) SH92HAC enzyme and “GmoSHC” may be used to refer to the Gluconobacter morbifer (Gmo) SHmsHAC enzyme.

AacSHC, ZmoSHC and BjpSHC enzyme sequences are disclosed in BASF WO 2010/139719, US 201201345477A1, Seitz et al (as cited above) and Seitz (2012 PhD thesis as cited above). Two different sequences are disclosed for ZmoSHC, referred to as ZmoSHC1 and ZmoSHC2. The Gino SHC/HAC enzyme sequence is disclosed in WO 2018/157021. The SaISHC enzyme is disclosed in Liu et al (2020): A Novel Soluble Squalene-Hopene Cyclase and Its Application in Efficient Synthesis of Hopene, Frontiers in Bioengineering and Biotechnology, vol 8, article 426, https:doi.orgal v.3389ifbioe.2020.00426).

TABLE 2
Sources and accession numbers of wild-type (WT) SHC enzymes.

| SeqID | Source organism | NCBI Acc. Nr.- |
|---|---|---|
| 69 | Aspergillus fumigatus | EDP50814.1 |
| 105 | Bacillus subtilis | AAB84441.1 |
| 19 | Bacillus anthracis | AAP27368.1 |
| 186 | Geobacter sulfurreducens | AAR34018.1 |
| 185 | Geobacter sulfurreducens | AAR36453.1 |
| 92 | Bacillus cereus ATCC 10987 | AAS42477.1 |
| 91 | Bacillus cereus E33L | AAU16998.1 |
| 96 | Bacillus licheniformis DSM 13 | AAU23777.1 |
| 13 | Zymomonas mobilis subsp. mobilis ZM4 ATCC | AAV90172.1 |
| | 31821 | |
| 191 | Gluconobacter oxydans | AAW61994.1 |
| 228 | Cupriavidus pinatubonensis JMP134 | AAZ64302.1 |
| 211 | Nitrobacter winogradskyi | ABA05523.1 |
| 65 | Anabaena variabilis | ABA24268.1 |
| 213 | Nitrosococcus oceani ATCC 19707 | ABA57818.1 |
| 30 | Pelobacter carbinolicus or Syntrophotalea | ABA87615.1 |
| | carbinolica DSM 2380 | |
| 223 | Syntrophotalea carbinolica DSM 2380 | ABA87701.1 |
| 142 | Burkholderia lata | ABB06563.1 |
| 143 | Burkholderia lata | ABB10136.1 |
| 178 | Geobacter metallireducens | ABB30662.1 |
| 179 | Geobacter metallireducens | ABB33038.1 |
| 216 | Nitrosospira multiformis ATCC 25196 | ABB75845.1 |
| 238 | Rhodospirillum rubrum ATCC 11170 | ABC20867.1 |
| 236 | Rhodopseudomonas palustris | ABD06434.1 |
| 167 | Frankia casuarinae | ABD10207.1 |
| 237 | Rhodopseudomonas palustris | ABD87279.1 |
| 145 | Burkholderia xenovorans LB400 | ABE35912.1 |
| 209 | Nitrobacter hamburgensis | ABE63461.1 |
| 230 | Ralstonia metallidurans | ABF11015.1 |
| 231 | Ralstonia metallidurans | ABF11268.1 |
| 146 | Candidatus Koribacter versatilis Ellin345 | ABF40741.1 |
| 239 | Rubrobacter xylanophilus DSM 9941 | ABG05671.1 |
| 268 | Trichodesmium erythraeum | ABG50159.1 |
| 214 | Nitrosomonas eutropha | ABI59752.1 |
| 192 | Granulibacter bethesdensis | ABI63005.1 |
| 29 | Burkholderia ambifaria | ABI91648.1 |
| 31 | Rhodopseudomonas palustris | ABJ08391.1 |
| 148 | Candidatus Solibacter usitatus Ellin6076 | ABJ82180.1 |
| 149 | Candidatus Solibacter usitatus Ellin6076 | ABJ82254.1 |
| 33 | Syntrophobacter fumaroxidans | ABK17672.1 |
| 33 | Syntrophobacter fumaroxidans | ABK18414.1 |
| 58 | Acidothermus cellulolyticus | ABK53469.1 |
| 106 | Bacillus thuringiensis str. Al Hakam | ABK86448.1 |
| 224 | Pelobacter propionicus DSM 2379 | ABK98395.1 |
| 225 | Pelobacter propionicus DSM 2379 | ABK98811.1 |
| 135 | Burkholderia mallei SAVP1 | ABM48844.1 |
| 187 | Geotalea uraniireducens Rf4 | ABQ25226.1 |
| 270 | Acidiphilium cryptum | ABQ30890.1 |
| 16 | Bradyrhizobium sp. | ABQ33590.1 |
| 95 | Bacillus cytotoxicus | ABS22481.1 |
| 66 | Anaeromyxobacter sp. | ABS28257.1 |
| 75 | Bacillus velezensis FZB42 | ABS74269.1 |
| 100 | Bacillus pumilus SAFR-032 | ABV62529.1 |
| 169 | Frankia sp. | ABW14125.1 |
| 168 | Frankia sp. | ABW15063.1 |
| 246 | Acaryochloris marina | ABW29816.1 |
| 137 | Burkholderia multivorans ATCC 17616 | ABX16859.1 |
| 136 | Burkholderia multivorans ATCC 17616 | ABX19650.1 |
| 114 | Bacillus mycoides KBAB4 | ABY44436.1 |
| 205 | Methylobacterium sp. | ACA20611.1 |
| 253 | Streptomyces peucetius subsp. caesius ATCC | ACA52082.1 |
| | 27952 | |
| 128 | Burkholderia cenocepacia | ACA95661.1 |
| 204 | Methylobacterium radiotolerans | ACB27373.1 |
| 155 | Crocosphaera subtropica ATCC 51142 | ACB53858.1 |
| 125 | Burkholderia ambifaria MC40-6 | ACB68303.1 |
| 203 | Methylobacterium populi | ACB79998.1 |
| 116 | Beijerinckia indica subsp. indica ATCC 9039 | ACB96717.1 |
| 138 | Burkholderia phymatum | ACC73258.1 |
| 217 | Nostoc punctiforme PCC 73102 | ACC84529.1 |
| 139 | Burkholderia phytofirmans PsJN | ACD21317.1 |
| 198 | Methylacidiphilum infernorum | ACD82457.1 |
| 177 | Geobacter lovleyi | ACD95949.1 |
| 234 | Rhodopseudomonas palustris | ACF02757.1 |
| 176 | Geobacter bemidjiensis | ACH40355.1 |
| 57 | Acidithiobacillus ferrooxidans | ACH84004.1 |
| 189 | Gluconacetobacter diazotrophicus | ACI51585.1 |
| 219 | Afipia carboxidovorans | ACI93782.1 |
| 206 | Methylocella silvestris | ACK52150.1 |
| 158 | Rippkaea orientalis PCC 8801 | ACK66841.1 |
| 156 | Gloeothece citriformis PCC 7424 | ACK71719.1 |
| 199 | Methylorubrum extorquens CM4 | ACK83067.1 |
| 200 | Methylorubrum extorquens | ACK86232.1 |
| 93 | Bacillus cereus G9842 | ACK95843.1 |
| 161 | Cyanothece sp. PCC 7425 | ACL45896.1 |
| 202 | Methylobacterium nodulans | ACL61886.1 |
| 180 | Geotalea daltonii FRC-32 | ACM21577.1 |
| 182 | Geotalea daltonii FRC-32. | ACM22003.1 |
| 34 | Teredinibacter turnerae | ACR13362.1 |
| 133 | Burkholderia glumae BGR1 | ACR30752.1 |
| 132 | Burkholderia glumae BGR1 | ACR32572.1 |
| 221 | Paenibacillus sp. JDR-2 | ACS99948.1 |
| 184 | Geobacter sp. M21 | ACT16952.1 |
| 150 | Catenulispora acidiphila DSM 44928 | ACU75510.1 |
| 240 | Saccharomonospora viridis DSM 43017 | ACU97316.1 |
| 162 | Rippkaea orientalis PCC 8802 | ACV02092.1 |
| 243 | Sphaerobacter thermophilus DSM 20745 | ACZ39437.1 |
| 309 | Komagataeibacter xylinus E25 | AHI26287.1 |
| 278 | Bacillus thuringiensis | AJI35613.1 |
| 286 | Komagataeibacter nataicola | AQU88860.1 |
| 267 | Synechocystis sp. PCC 6803 | BAA17978.1 |
| 218 | Nostoc sp. PCC 7120 | BAB72732.1 |
| 23 | Thermosynechococcus elongatus | BAC09861.1 |
| 245 | Streptomyces avermitilis NBRC 14893 | BAC69361.1 |
| 188 | Gloeobacter violaceus PCC 7421 | BAC91998.1 |
| 74 | Aspergillus oryzae | BAE63941.1 |
| 59 | Adiantum capillus-veneris | BAF93209.1 |
| 251 | Streptomyces griseus ATCC 53653 | BAG17791.1 |
| 164 | Dryopteris crassirhizoma | BAG68223.1 |
| 122 | Brevibacillus brevis NBRC 100599 | BAH44778.1 |
| 120 | Acetobacter pasteurianus IFO 3283-01 | BAH98349.1 |
| 24 or 27 | Acetobacter pasteurianus | BAH99456.1 |
| 227 | Goniophlebium niponicum | BAI48070.1 |
| 226 | Goniophlebium niponicum | BAI48071.1 |
| 119 | Bradyrhizobium japonicum | CAA60250.1 |
| 64 | Alicyclobacillus acidoterrestris | CAA61950.1 |
| 207 | Methylococcus capsulatus | CAA71098.1 |
| 235 | Rhodopseudomonas palustris | CAA71101.1 |
| 32 | Streptomyces coelicolor | CAB39697.1 |
| 233 | Rhodopirellula baltica | CAD74517.1 |
| 166 | Frankia alni ACN14a | CAJ60090.1 |
| 165 | Frankia alni ACN14a | CAJ61140.1 |
| 147 | Candidatus Kuenenia stuttgartiensis | CAJ71215.1 |
| 229 | Cupriavidus necator H16 | CAJ96989.1 |
| 72 | Aspergillus niger | CAK43501.1 |
| 121 | Bradyrhizobium sp. ORS 278 | CAL79893.1 |
| 241 | Saccharopolyspora erythraea NRRL 2338 | CAM03596.1 |
| 190 | Gluconacetobacter diazotrophicus | CAP55563.1 |
| 154 | Cupriavidus taiwanensis LMG 19424 | CAQ72562.1 |
| 130 | Burkholderia cenocepacia J2315 | CAR56694.1 |
| 129 | Burkholderia cenocepacia J2315 | CAR57099.1 |
| 201 | Methylobacterium extorquens | CAX24364.1 |
| 255 | Streptomyces scabiei | CBG68454.1 |
| 284 | Gluconacetobacter xylinus | CUW48332.1 |
| 287 | Komagataeibacter xylinus | AHI26287.1 |
| 77 | Bacillus cereus G9241 | EAL12758.1 |
| 70 | Aspergillus fumigatus | EAL84865.1 |
| 71 | Aspergillus fumigatus | EAL86291.2 |
| 153 | Crocosphaera watsonii WH 8501 | EAM53094.1 |
| 210 | Nitrobacter sp. | EAQ34404.1 |
| 118 | Blastopirellula marina DSM 3645 | EAQ78122.1 |
| 117 | Blastopirellula marina DSM 3645 | EAQ81955.1 |
| 212 | Nitrococcus mobilis | EAR22397.1 |
| 102 | Bacillus sp. NRRL B-14911 | EAR64404.1 |
| 67 | Aspergillus clavatus | EAW07713.1 |
| 208 | Neosartorya fischeri NRRL 181 | EAW20752.1 |
| 195 | Leptospirillum rubarum. | EAY57382 |
| 127 | Burkholderia cenocepacia PC184 | EAY66961.1 |
| 131 | Burkholderia dolosa AU0158 | EAY71311.1 |
| 160 | Crocosphaera chwakensis | EAZ91809.1 |
| 103 | Bacillus sp. SG-1 | EDL66148.1 |
| 69 | Aspergillus fumigatus | EDP50814.1 |
| 220 | Paenibacillus sp. | EDS49994.1 |
| 123 | Burkholderia ambifaria IOP40-10 | EDT05097.1 |
| 134 | Burkholderia graminis C4D1M | EDT12320.1 |
| 124 | Burkholderia ambifaria MEX-5 | EDT37649.1 |
| 126 | Burkholderia ambifaria MEX-5 | EDT42454.1 |
| 181 | Geobacter sp. M21 | EDV72707.1 |
| 101 | Bacillus pumilus ATCC 7061 | EDW21137.1 |
| 256 | Streptomyces sp. | EDX25760.1 |
| 265 | Synechococcus sp. PCC 7335 | EDX84551.1 |
| 159 | Cyanothece sp. PCC 7822 | EDX97382.1 |
| 157 | Cyanothece sp. PCC 8802 | EDY02410.1 |
| 173 | Geobacillus thermodenitrificans | EDY05760.1 |
| 151 | Chthoniobacter flavus Ellin428 | EDY15838.1 |
| 152 | Chthoniobacter flavus Ellin428 | EDY22035.1 |
| 257 | Streptomyces sp. | EDY46371.1 |
| 263 | Streptomyces sviceus ATCC 29083 | EDY55942.1 |
| 254 | Streptomyces pristinaespiralis ATCC 25486 | EDY61772.1 |
| 196 | Leptospirillum sp. | EDZ38599.1 |
| 141 | Burkholderia sp. H160 | EEA03553.1 |
| 242 | Schizosaccharomyces japonicus | EEB08219.1 |
| 140 | Burkholderia pseudomallei | EEC32728.1 |
| 163 | Desulfovibrio salexigens DSM 2638 | EEC62384.1 |
| 62 | Alicyclobacillus acidocaldarius LAA1 | EED08231.1 |
| 68 | Aspergillus flavus | EED48353.1 |
| 172 | Geobacillus sp. | EED61885.1 |
| 115 | Pedosphaera parvula Ellin514 | EEF59508.1 |
| 61 | Histoplasma capsulatum G186AR | EEH02950.1 |
| 79 | Bacillus cereus m1293 | EEK43841.1 |
| 76 | Bacillus wiedmannii | EEK66523.1 |
| 82 | Bacillus cereus | EEK77935.1 |
| 80 | Bacillus cereus | EEK82938.1 |
| 84 | Bacillus cereus m1550 | EEK88307.1 |
| 81 | Bacillus cereus | EEK99528.1 |
| 78 | Bacillus cereus Rock3-42 | EEL44583.1 |
| 90 | Bacillus cereus Rock3-44 | EEL49968.1 |
| 86 | Bacillus cereus Rock4-18 | EEL59884.1 |
| 85 | Bacillus cereus F65185 | EEL63943.1 |
| 88 | Bacillus cereus | EEL69857.1 |
| 83 | Bacillus cereus | EEL81079.1 |
| 89 | Bacillus cereus AH1273 | EEL92663.1 |
| 97 | Bacillus mycoides DSM 2048 | EEL98438.1 |
| 98 | Bacillus pseudomycoides | EEM04821.1 |
| 99 | Bacillus pseudomycoides DSM 12442 | EEM16144.1 |
| 107 | Bacillus thuringiensis serovar tochigiensis BGSC | EEM21409.1 |
| | 4Y1 | |
| 110 | Bacillus thuringiensis Bt407 | EEM27851.1 |
| 111 | Bacillus thuringiensis serovar sotto str. T04001 | EEM40716.1 |
| 112 | Bacillus thuringiensis serovar pakistani str. | EEM46814.1 |
| | T13001 | |
| 109 | Bacillus thuringiensis serovar kurstaki str. | EEM52372.1 |
| | T03a001 | |
| 108 | Bacillus thuringiensis serovar huazhongensis | EEM82653.1 |
| | BGSC 4BD1 | |
| 113 | Bacillus thuringiensis IBL 200 | EEM94969.1 |
| 174 | Geobacillus sp. Y412MC52 | EEN95021.1 |
| 175 | Geobacillus sp. Y412MC61 | ACX79399.1 |
| 60 | Histoplasma capsulatum H143 | EER40510.1 |
| 308 | Zymomonas mobilis | EER62728.1 |
| 194 | Leptospirillum ferrodiazotrophum | EES53667.1 |
| 222 | Paenibacillus sp. oral taxon 786 | EES74793.1 |
| 56 | Acidithiobacillus caldus | EET25937.1 |
| 215 | Nitrosomonas sp. AL212 | EET32702.1 |
| 183 | Geobacter sp. M18 | EET34621.1 |
| 193 | Hyphomicrobium denitrificans ATCC 51888 | EET65847.1 |
| 248 | Streptomyces flavogriseus ATCC 33331 | EEW70811.1 |
| 144 | Burkholderia sp. CCGE1002 | EFA54357.1 |
| 171 | Frankia sp. Eullc | EFA59089.1 |
| 170 | Frankia sp. Eullc | EFA59873.1 |
| 285 | Gluconobacter frateurii NBRC 103465 | GAD08844.1 |
| 272 | Acetobacter orleanensis JCM 7639 | GAN69910.1 |
| 272 | Acetobacter orleanensis JCM 7639 | GAN69910.1 |
| 293 | Thermoactinomyces sp. Gus2-1 | KFZ40906.1 |
| 285 | Gluconobacter frateurii NBRC 103465 | GAD08844.1 |
| 295 | Alicyclobacillus acidocaldarius | P33247.4 |
| 232 | Rhizobium sp. | P55348.1 |
| 271 | Acetobacter fabarum | PAK78064.1 |
| 104 | Bacillus subtilis | Q796C3.1 |
| 277 | Bacillus subtilis | WP_004399534.1 |
| 288 | Methylacidiphilum fumariolicum | WP_009061034.1 |
| 289 | Methylococcus capsulatus | WP_010960137.1 |
| 23 | Thermosynechococcus elongatus | WP_011058142.1 |
| 294 | Zymomonas mobilis | WP_011241313.1 |
| 29 | Syntrophobacter fumaroxidans | WP_011698842.1 |
| 274 | Acidothermus cellulolyticus | WP_011720532.1 |
| 275 | Alicyclobacillus acidocaldarius | WP_012811690.1 |
| 273 | Acetobacter pasteurianus | WP_012812952.1 |
| 290 | Sphaerobacter thermophiles | WP_012872483.1 |
| 276 | Bacillus megaterium | WP_013083001.1 |
| 280 | Chloracidobacterium thermophilum | WP_014100779.1 |
| 292 | Teredinibacter turnerae | WP_015819476.1 |
| 281 | Cohnella thermotolerans | WP_027091823.1 |
| 279 | Brevibacillus thermoruber | WP_029099368.1 |
| 282 | Geobacillus thermodenitrificans | WP_029761705.1 |
| 310 | Geobacillus vulcani | WP_031409036 |
| 283 | Geobacillus sp. | WP_031409036.1 |
| 197 | Magnetospirillum magnetotacticum | ZP_00052068.2 |
| 269 | Verrucomicrobium spinosum DSM 4136 | ZP_02925563.1 |
| 94 | Bacillus coahuilensis | ZP_03226483.1 |
| 249 | Streptomyces ghanaensis ATCC 14672 | ZP_04684415.1 |
| 247 | Streptomyces roseosporus NRRL 15998 | ZP_04697579.1 |
| 244 | Streptomyces albus | ZP_04706071.1 |
| 261 | Streptomyces sp. SPB74 | ZP_04995097.1 |
| 260 | Streptomyces sp. Mg1 | ZP_05001249.1 |
| 266 | Synechococcus sp. PCC 7335 | ZP_05035816.1 |
| 258 | Streptomyces sp. AA4 | ZP_05479203.1 |
| 262 | Streptomyces sp. SPB78 | ZP_05490781.1 |
| 259 | Streptomyces sp. | ZP_05510126.1 |
| 252 | Streptomyces lividans | ZP_05522220.1 |
| 264 | Streptomyces viridochromogenes DSM 40736 | ZP_05535608.1 |
| 250 | Streptomyces griseoflavus | ZP_05537325.1 |
| 307 | Gluconobacter morbifer | EHH69691.1 |
| 55 | Acetobacter pasteurianus | IFO 3283-01 |
| 63 | Alicyclobacillus acidocaldarius | AAT70690.1 |
| 73 | Aspergillus niger | CAK45506.1 |
| 87 | Bacillus cereus Rock4-18 | EEL59884.2 |

TABLE 3
Sources and accession numbers of preferred wild-type (WT) SHC enzymes.

| | | | SEQ ID No. |
|---|---|---|---|
| | | | according to |
| | Reference | | WO 2010139719 |
| | (and incorporated | | US2012/0135477 (*) |
| SHC Source Strain | herein by | | or to the present |
| (SHC name) | reference) | Accession No. | disclosure (**) |
| Alicyclobacillus acidocaldarius | JP2009-060799 | NBRC15652 | SEQ ID No: 1 ** |
| (AacSHC) | (Kao) | | |
| | Neumann et al Biol | | |
| | Chem (1986) 367; | | |
| | 723-729 | | |
| Zymomonas mobilis (ZmoSHC1) | WO2010139719 | ATCC31821 | SEQ ID No. 1 * |
| | US20120135477 | PF62207_2 | SEQ ID NO: 13 ** |
| | | Genpept | SEQ ID No. 2 * |
| | | Accession No. | SEQ ID NO: 14 ** |
| | | AAV90172 | |
| | | AAF12829.1 | |
| | | AVZ42714.1 | |
| Zymomonas mobilis (ZmoSHC2) | Reipen et al (1995) | EMBL/Genbank | SEQ ID No. 15 ** |
| | Microbiology | Accession No. | |
| | 141: 155-161 | X80766 | |
| Bradryhizobium japonicum (BjpSHC | WO2010139719 | PF62207_5 | SEQ ID No. 5 * |
| or BjaSHC) | US2012/0135477 | ABQ33590.1 | SEQ ID NO: 16 ** |
| Burkholderia ambifaria | WO2010139719 | | SEQ ID No. 6 * |
| | US2012/0135477 | | SEQ ID NO: 17 ** |
| Burkholderia ambifaria | WO2010139719 | | SEQ ID No. 7 * |
| | US2012/0135477 | | SEQ ID NO: 18 ** |
| Bacillus anthracis | WO2010139719 | | SEQ ID No. 8 * |
| | US2012/0135477 | | SEQ ID NO: 19 ** |
| Frankia alni | WO2010139719 | | SEQ ID No. 9 * |
| | US2012/0135477 | | SEQ ID NO: 20 ** |
| Rhodopseudomonas palustris | WO2010139719 | | SEQ ID No. 10 * |
| | US2012/0135477 | | SEQ ID NO: 21 ** |
| Gluconobacter morbifer (WT | WO2018157021 | EHH69691.1 | SEQ ID NO: 22 ** |
| GmoSHC) | | | |
| Thermosynechococcus elongatus | | BAC09861.1 | SEQ ID NO: 23 ** |
| (TelSHC) | | | |
| Acetobacter pasteurianus | | ASC07046.1 | SEQ ID NO: 24 ** |
| (ApaSHC1) | | | |
| Bacillus megaterium (BmeSHC) | | WP_016763969 | SEQ ID NO: 25 ** |
| Streptomyces albolongus SalSHC) | | AZN28579 | SEQ ID NO: 26 ** |
| Acetobacter pasteurianus | | WP_003625617 | SEQ ID NO: 27 ** |
| ApaSHCA) | | | |
| Burkholderia ambifaria BamSHC1) | | AJY25620.1 | SEQ ID NO: 28 ** |
| Burkholderia ambifaria (BamSHC2) | | ABI91648.1 | SEQ ID NO: 29 ** |
| Pelobacter carbinolicus (PcaSHC2) | | ABA87615.1 | SEQ ID NO: 30 ** |
| or Syntrophotalea carbinolica DSM | | | |
| 2380 | | | |
| Rhodopseudomonas palustris | | ABJ08391.1 | SEQ ID NO: 31 ** |
| (RpaSHC1) | | | |
| Streptomyces coelicolor (ScoSHC) | | CAB39697.1 | SEQ ID NO: 32 ** |
| Syntrophobacter fumaroxidans | | ABK17672.1 | SEQ ID NO: 33 ** |
| (SfuSHC) | | | |
| Teredinibacter turnerae (TtuSHC) | | ACR13362.1 | SEQ ID NO: 34 ** |

The sequences of the wild-type AacSHC, wild-type ZmoSHC1, wild-type ZmoSHC2, wild-type BjpSHC, wild-type GmoSHC, wild-type TeISHC and wild-type ApaSHC1, wild-type BmeSHC, wild-type SaISHC and wild-type ApaSHCA, are also disclosed herein in table 3.

Most preferred wild-type SHC are: AacSHC (SEQ ID NO: 1) or ZmoSHC1 represented by SEQ ID NO:13 or ZmoSCH2 represented by SEQ ID NO:15 or Tel SHC represented by SEQ ID NO:23 or ScoSHC represented by SEQ ID NO:32.

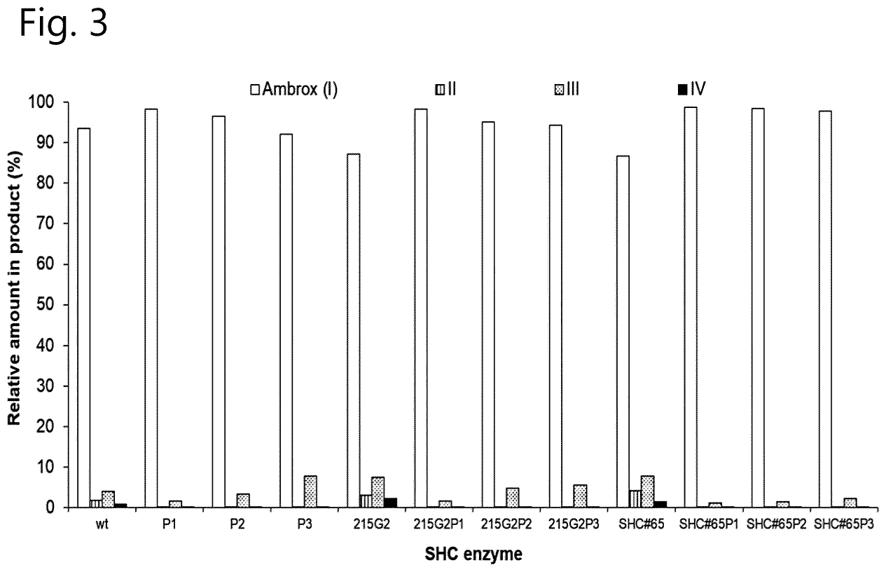

An alignment of WT SHC sequences prepared by Hoshino and Sato (2002 as cited above) indicates that multiple motifs were detected and consists of the core sequence Gln-X-X-X-Gly-X-Trp which is found six times in the SHC sequences of both Z. mobilis and A. acidocaldarius (See FIG. 3 of Reipen et al 1995, Microbiology 141, 155-161) (X can be any amino acid). Hoshino and Sato (2002 as cited above) report that aromatic amino acids are unusually abundant in SHCs and that two characteristic motifs were noted in the SHCs: one is a QW motif represented by specific amino acid motifs [(K/R)(G/A)X2-3(F/Y/W)(L/IV)3X3QX2-5GXW] and the alternative is a DXDDTA motif (SEQ ID NO:314) Wendt et al (1997, Science 277, 1811-1815 and 1999, J Mol Biol 286, 175-187) reported on the X-ray structure analysis of A. acidocaldarius SHC (X can be any amino acid). The DXDDTA motif appears to correlate with the SHC active site.

A reference AacSHC protein as used herein may refer to the wild-type AacSHC protein as disclosed in SEQ ID NO: 1. AacSHC has the activity of a homofarnesol Ambrox cyclase (HAC) useful in the production of Ambrox derivatives through a biocatalytic reaction of SHC with a homofarnesol substrate. The main reaction of the AacSHC is the cyclisation of a linear or a non-linear substrate such as homofarnesol to produce Ambrox.

Another reference AacSHC protein is SHC/AHC enzyme 215G2 SHC or 215G2 SHC as disclosed in SEQ ID NO: 3. SEQ ID NO:3 corresponds to SEQ ID NO:1 with the mutations M132R, A224V and 1432T.

Another reference AacSHC protein is SHC/AHC enzyme variant #65 as disclosed in SEQ ID NO: 2. SEQ ID NO:2 corresponds to SEQ ID NO: 1 with the substitutions M132R, A224V, 1432T, A557T and R613S and may be referred to as SHC/HAC enzyme variant #65 or SHC #65 variant herein.

The wording “functional homologs” may be replaced by “functional equivalents” or by “homologs”.

Functional homologs of the wild-type SHC/HAC enzymes or the SHC/HAC enzyme variants described herein are also suitable for use in cyclization reactions, for example for producing (−)-Ambrox, for example in a recombinant host. Thus, the recombinant host may include one or more heterologous nucleic acid(s) encoding functional homologs of the polypeptides described above and/or a heterologous nucleic acid encoding a SHC/HAC derivative enzyme as described herein.

A functional homolog is a polypeptide that has sequence identity and optionally sequence similarity to a reference polypeptide, and that carries out one or more of the biochemical or physiological function(s) of the reference polypeptide. A functional homolog and the reference polypeptide may be natural occurring polypeptides, and the sequence identity and optionally sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild-type coding sequence, may themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional homologs described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide:polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of the nucleic acid sequences encoding the SHC derivative polypeptides and the like.

Hybridization can also be used to identify functional homologs and/or as a measure of homology between two nucleic acid sequences. A nucleic acid sequence encoding any of the proteins disclosed herein, or a portion thereof, can be used as a hybridization probe according to standard hybridization techniques. The hybridization of a probe to DNA or RNA from a test source (e.g. a mammalian cell) is an indication of the presence of the relevant DNA or RNA in the test source.

Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C. followed by a wash in 1×SSC, 0.1% SDS at 50° C. Highly stringent conditions are defined as equivalent to hybridization in 6× sodium chloride/sodium citrate (SSC) at 45° C. followed by a wash in 0.2×SSC, 0.1% SDS at 65° C. Sequence analysis to identify functional homologs can also involve BLAST, Reciprocal BLAST, or PSI-BLAST analysis of non-redundant databases using a relevant amino acid sequence as the reference sequence. Amino acid sequence is, in some instances, deduced from the nucleotide sequence. Those polypeptides in the database that have greater than 40% sequence identity are candidates for further evaluation for suitability for use in the SHC/HAC bioconversion reaction. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have for e.g. conserved functional domains.

Typically, polypeptides that exhibit at least about 30% amino acid sequence identity are useful to identify conserved regions. Conserved regions of related polypeptides exhibit at least 30%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, amino acid sequence identity. In some embodiments, a conserved region exhibits at least, 70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity. Sequence identity can be determined as set forth above and below. Usually in this context, a region may comprise from 5 to 50 amino acids or from 10 to 150 or from 10 to 80 or from 10 to 100 amino acids.

The SHC/HAC enzymes or enzyme variants described herein and used in the methods described herein may, for example, be based on an amino acid sequence of SEQ ID NO: 1, SEQ ID NO: 2, SEQ ID NO: 3, SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or of any of SEQ ID NO: 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or a variant, homologue, mutant, derivative or fragment thereof.

In addition, the produced reference SHC enzyme may be based on an amino acid sequence produced from E. coli.

“Percent (%) identity” with respect to the nucleotide sequence of a gene is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared. The terms “polypeptide” and “protein” are used interchangeably herein and mean any peptide-linked chain of amino acids, regardless of length or post-translational modification.

As used herein the term “derivative” includes but is not limited to a variant. The terms “derivative” and “variant” are used interchangeably herein.

In preferred embodiments, a variant enzyme usable in the present disclosure exhibits a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes (alterations) in the amino acid sequence (i.e. exchanges, insertions, deletions, N-terminal truncations, and/or C-terminal truncations). The amino acid exchanges may be conservative and/or non-conservative. In preferred embodiments, a variant usable in the present disclosure differs from the protein or domain from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acid exchanges, preferably conservative amino acid changes. Variants may additionally or alternatively comprise deletions of amino acids, which may be N-terminal truncations, C-terminal truncations or internal deletions or any combination of these. Such variants comprising N-terminal truncations, C-terminal truncations and/or internal deletions are referred to as “deletion variants” or “fragments” in the context of the present application. The terms “deletion variant” and “fragment” are used interchangeably herein. A deletion variant may be naturally occurring (e.g. splice variants) or it may be constructed artificially, preferably by gene-technological means. Typically, the protein or protein domain from which the deletion variant is derived is a wild-type protein. However, the deletion variants of the present disclosure may also be derived from homologs, orthologs, or paralogs of the parent polypeptide or from artificially constructed variants, provided that the deletion variants exhibit at least one biological activity of the parent polypeptide. Preferably, a deletion variant (or fragment) has a deletion of up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acids at its N-terminus and/or at its C-terminus and/or internally as compared to the parent polypeptide.

In certain embodiments, the SHC/HAC enzyme variants described herein only include substitutions and do not include any deletions or insertions.

A “variant” as used herein, can alternatively or additionally be characterised by a certain degree of sequence identity or similarity to the parent polypeptide from which it is derived. A variant of the WT/reference SHC/HAC or the SHC/HAC Derivative of the present disclosure may have a sequence identity of at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the respective reference polypeptide or to the respective reference polynucleotide.

The expression “at least 30%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity” is used throughout the specification with regard to polypeptide and polynucleotide sequence comparisons. A polynucleotide belonging to a family of any of the enzymes disclosed herein or a protein can be identified based on its similarity to the relevant gene or protein, respectively. For example, the identification can be based on sequence identity. In certain preferred embodiments the disclosure features isolated nucleic acid molecules which are at least 30%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to (a) a nucleic acid molecule that encodes the polypeptide of a wild-type SHC/HAC enzyme (e.g. SEQ ID NO: 1, SEQ ID NO:13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or any other SHC/HAC disclosed in table 2, 3 or 16 disclosed herein (b) the nucleotide sequence SEQ ID NO: 35 and (c) a nucleic acid molecule which includes a segment of at least 30 (e.g. at least 30, 40, 50, 60, 80, 100, 125, 150, 175, 200, 250, 300, 400, 500, 600, 700, 800, 850, 900, 950, 1000, or 1010) nucleotides of SEQ ID NO: 35 (or a nucleotide sequence encoding a SHC/HAC enzyme as disclosed in table 2, 3 or 16).

Preferably, the polypeptide in question and the reference polypeptide exhibit the indicated sequence identity or similarity over a continuous stretch of 20, 30, 40, 45, 50, 60, 70, 80, 90, 100 or more amino acids. Preferably, the polynucleotide in question and the reference polynucleotide exhibit the indicated sequence identity over a continuous stretch of 60, 90, 120, 135, 150, 180, 210, 240, 270, 300 or more nucleotides. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence (e.g. SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 and any SHC/HAC enzyme as disclosed in table 2, 3 or 16) if not specifically indicated otherwise.

For example, a peptide sequence consisting of 130 amino acids compared to the amino acids of full length of wild-type AacSHC with 631 amino acid residues may exhibit a maximum sequence identity percentage of 20.6% (130/631×100) while a sequence with a length of 300 amino acids may exhibit a maximum sequence identity percentage of 47.5% (300/631×100).

The identity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on https://www.ebi.ac.uk/Tools/msa/clustalo/or the GAP program (mathematical algorithm of the University of Iowa) or the mathematical algorithm of Myers and Miller (1989—Cabios 4: 11-17) or Clone Manager 9. Preferred parameters used are the default parameters as they are set on https://www.ebi.ac.uk/Tools/msa/clustalo/.

The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215, 403-410. BLAST polynucleotide searches are performed with the BLASTN program, score=100, word length=12, to obtain polynucleotide sequences that are homologous to those nucleic acids which encode the relevant protein.

BLAST protein searches are performed with the BLASTP program, score=50, word length=3, to obtain amino acid sequences homologous to the SHC polypeptide. To obtain gapped alignments for comparative purposes, Gapped BLAST is utilized as described in Altschul et al (1997) Nucleic Acids Res. 25, 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1: 154-162) or Markov random fields. When percentages of sequence identity are referred to in the present application, these percentages are calculated in relation to the full length of the longer sequence, if not specifically indicated otherwise.

In particular embodiments, % identity between two sequences is determined using CLUSTAL O (version 1.2.4).

In an embodiment, “similarity” means the degree of sequence relatedness between amino acid sequences, as the case may be, as determined by the match between strings of such sequences.

“Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Optionally, in determining the degree of amino acid similarity, the skilled person may also take into account so-called “conservative” amino acid substitutions, as will be clear to the skilled person. “Similarity” between two amino acid sequences is determined by comparing the amino acid sequence and its conserved amino acid substitutes of one polypeptide to the sequence of a second polypeptide. As used herein, “conservative” amino acid substitutions refer to the interchangeability of residues having similar side chains. Examples of classes of amino acid residues for conservative substitutions are given in the Tables below.

TABLE 4
Classes of amino acid residues

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), and |
| Residues | Gln (Q) |
| Aliphatic Uncharged | Gly (G), Ala (A), Val (V), Leu (L), |
| Residues | and Ile (I) |
| Non-polar Uncharged Residues | Cys (C), Met (M), and Pro (P) |
| Aromatic Residues | Phe (F), Tyr (Y), and Trp (W) |

TABLE 5
Alternative conservative amino acid residue substitution classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

TABLE 6
Alternative physical and functional classifications
of amino acid residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, |
| | V, W, and Y |
| Negatively charged residues | D and E |
| Polar residues | C, D, E, H, K, N, Q, R, S, |
| | and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, |
| formation | S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lie or Val; Lys to Arg; Gln or Glu; Met to Leu or lie; Phe to Met, Leu or Tyr; Ser to Thr; Thr to Ser; Trp to Tyr; Tyr to Trp or Phe; and, Val to lie or Leu.

Specific SHC/HAC enzymes and enzymes variants that may be used in the methods described herein are further described below.

Variants of SHC/HAC with New Mutations

It has surprisingly been found that SHC/HAC enzyme variants derived from Aac SHC/HAC enzyme (SEQ ID NO:1) or from variants of said Aac SHC/HAC enzyme (SEQ ID NO:2 or SEQ ID NO:3) or derived from other wild type SHC as identified herein (such as those identified in table 2, 3 or 16 especially such as those represented by SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) or derived from other variants (such as those represented by SEQ ID NO: 47, 48, 49, 50, 51, 52, 53, 54) and those disclosed in Table 14 of WO2016/170099 and/or Table 6 of WO2021/110848 (as provided below as represented by SEQ ID NO: 361, 362, 363, 364, 365, 366, 367, 368, 369, 370, 371, 372, 373, 374, 375, 376, 377, 378, 379, 380, 381, 382 or 383) exhibit modified/optimised activities/biological activities as earlier defined herein and as further demonstrated in the experimental part.

SEQ ID NO:3 corresponds to SEQ ID NO:1 with the amino acid alterations (substitutions) M132R, A224V and 1432T.

SEQ ID NO:2 corresponds to SEQ ID NO: 1 with the amino acid alterations (substitutions) M132R, A224V, 1432T, A557T and R613S.

In an embodiment, the new SHC/HAC enzyme variants derived from SEQ ID NO:2 or 3 still comprise the amino acid alterations (substitutions) they had compared to SEQ ID NO:1. These amino acid alterations are the following:
Variant derived from SEQ ID NO:3 may comprise the following amino acid alterations (substitutions): M132R, A224V and 1432T compared to SEQ ID NO:1,
Variant derived from SEQ ID NO:2 may comprise the following amino acid alterations (substitutions): M132R, A224V, 1432T, A557T and R613S compared to SEQ ID NO:1,
Variant derived from SEQ ID NO:2 may comprise the following amino acid alterations (substitutions): M132R and 1432T compared to SEQ ID NO:1,
Variant derived from SEQ ID NO:2 may comprise the following amino acid alterations (substitutions): M132R and 1432T, A557T and R613S compared to SEQ ID NO:1.

Variant derived from any one of the sequences listed in Table 14 of WO2016/170099 including but not limited to SEQ ID No. 5, 7, 9, 11, 13, 15, 17, 19, 171, 23, 25, 27, 29, 31, 33, 35, 37 or 39 (corresponding to SEQ ID NO:361-368, 378, 3, 369-377 of the present application respectively). (see table 7 below and sequences disclosed at the end of the experimental part) Variants derived from any one of the sequences listed in Table 6 of WO2021/110848 including but not limited to SEQ ID No. 5, 17, 18, 2 or 4 (see table 8 below and sequences disclosed at the end of the experimental part).

TABLE 7
corresponding to Table 14 of WO 2016/170099: AacSHC
Derivative amino acid and nucleotide SEQ ID No.

| | | SHC | |
|---|---|---|---|
| | | Deriva- | Nucleotide |
| Amino acid | | tive | SEQ ID |
| SEQ ID No. | Mutation (s) | name | No. |
| SEQ ID No. 5 | T77A | | 6 |
| SEQ ID No. 7 | I92V | | 8 |
| SEQ ID No. 9 | F129L | | 10 |
| SEQ ID No. 11 | M132R | | 12 |
| SEQ ID No. 13 | A224V | | 14 |
| SEQ ID No. 15 | I432T | | 16 |
| SEQ ID No. 17 | Q579H | | 18 |
| SEQ ID No. 19 | F601Y | | 20 |
| SEQ ID No. 171 | F605W | | 170 |
| SEQ ID No. 21 | M132R + A224V + I432T | 215G2 | 22 |
| SEQ ID No. 23 | M132R + I432T | SHC26 | 24 |
| SEQ ID No. 25 | F601Y | SHC3 | 26 |
| SEQ ID No. 27 | T77A + I92V + F129L | 111C8 | 28 |
| SEQ ID No. 29 | Q579H + F601Y | 101A10 | 30 |
| SEQ ID No. 31 | F129L | SHC10 | 32 |
| SEQ ID No. 33 | F129L + F601Y | SHC30 | 34 |
| SEQ ID No. 35 | F129L + M132R + I432T | SHC31 | 36 |
| SEQ ID No. 37 | M132R + I432T + F601Y | SHC32 | 38 |
| SEQ ID No. 39 | F129L + M132R + I432T + | SHC33 | 40 |
| | F601Y | | |

TABLE 8
corresponding to Table 6 of WO2021/110848. Mutations
in selected new SHC variant enzymes.

| SHC Variant | Y81H | T90A | A172T | M277K | H431L | A557T | R613S |
|---|---|---|---|---|---|---|---|
| 110B8 | + | | | | + | + | |
| (SEQ ID NO: 5) | | | | | | | |
| 90C7 | | + | | | | | + |
| (SEQ ID NO: 17) | | | | | | | |
| 115A7 | | | + | + | | | |
| (SEQ ID NO: 18) | | | | | | | |
| SHC 49 | | | | | + | + | |
| (SEQ ID NO: 2) | | | | | | | |
| SHC 65 | | | | | | + | + |
| (SEQ ID NO: 3) | | | | | | | |
| SHC 66 | + | | | | | + | + |
| (SEQ ID NO: 4) | | | | | | | |

Note:
these mutations appear in addition to the mutations present in 215G2 SHC: M132R, A224V, and I432T. SEQ ID NO: 2, 3, 4, 5, 17 and 18 of table 8 correspond to SEQ ID NO: 379, 2, 380, 381, 382 and 383.

Sequences identified in tables 7 and 8 above are represented by any of SEQ ID NO: 361, 362, 363, 364, 365, 366, 367, 368, 369, 370, 371, 372, 373, 374, 375, 376, 377, 378, 379, 380, 381, 382 or 383 in the sequence listing and as indicated at the end of the experimental part.

In certain embodiments, the SHC/HAC enzyme variant may have equal to or less than about 30 amino acid alterations compared to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383. For example, the SHC/HAC enzyme variant may have equal to or less than about 25 or equal to or less than about 20 or equal to or less than about 15 or equal to or less than about 10 or equal to or less than about 9 or equal to or less than about 8 or equal to or less than about 7 or equal to or less than about 6 amino acid alterations compared to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383. For example, the SHC/HAC enzyme variant may have at least about 5 or at least about 6 amino acid alterations compared to SEQ ID NO: 1, 2, 3, 13, 14, 15, 16, 17, 18, 19, 20, 21, 22, 23, 24, 25, 26, 27, 28, 29, 30, 31, 32, 33, 34, 55. 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383. The amino acid alterations may, for example, be insertions, deletions and/or substitutions as described above.

In certain embodiments, the only amino acid alterations in the SHC/HAC enzyme variant compared to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 are substitutions (i.e. there are no insertions or deletions).

Amino acid alterations are defined relative to a reference sequence. An amino acid alteration relative to a reference sequence means that the amino acid sequence of the variant sequence is different to the reference sequence.

Amino acids in the reference sequence and the variant sequence may be assigned a number, where the numbering starts with the amino acid at the N-terminus of the polypeptide (i.e. the amino acid at the N-terminus of the polypeptide is numbered 1, the next amino acid is numbered 2 etc.). The “position” of a reference sequence refers to a specific amino acid residue present in the reference sequence as identified by the specific numbering of the amino acids in the reference sequence. The “position” of a variant sequence refers to a specific amino acid residue present in the variant sequence as identified by the specific numbering of the amino acids in the variant sequence.

Since the variant sequence may include deletions or insertions compared to the reference sequence, the amino acids in the variant sequence may be numbered differently to the same amino acids in the reference sequence. By way of example, if an amino acid is inserted between amino acids 131 and 132 of SEQ ID NO: 1, the amino acid following the insertion will have the numbering 133 in the variant sequence while it retains the numbering 132 in the reference sequence. In this example, the position of the variant sequence that corresponds to position 132 of the reference sequence is position 133. Therefore, amino acids in the variant sequence that have been retained from the reference sequence may be defined by referring to the “corresponding position” of the reference sequence. In other words, a “position” in the variant sequence may be defined by reference to a “corresponding position” in the reference sequence.

In particular, substitutions in the variant sequence compared to the reference sequence may be defined by referring to the “corresponding position” of the reference sequence in spite of any insertions and/or deletions in the reference sequence. Where the amino acids of a reference sequence have been deleted, there is no “corresponding position” in the variant sequence. Where there are no insertions or deletions compared to the reference sequence (i.e. there are only substitutions), the “corresponding position” of the reference sequence will be the same as the position in the variant sequence.

For example, position 169 in AacSHC represented by SEQ ID NO:1 corresponds to position 222 in ZmoSHC1 (SEQ ID NO:13 or 14), position 177 in ZmoSHC2 (SEQ ID NO:15), position 172 in TeISHC (SEQ ID NO:23) and position 196 in ScoSH1 (SEQ ID NO:32).

For example, position 306 in AacSHC represented by SEQ ID NO:1 corresponds to position 368 in ZmoSHC1 (SEQ ID NO:13 or 14), position 321 in ZmoSHC2 (SEQ ID NO:15), position 311 in TeISHC (SEQ ID NO:23) and position 335 in ScoSH1 (SEQ ID NO:32).

For example, position 600 in AacSHC represented by SEQ ID NO:1 corresponds to position 667 in ZmoSHC1 (SEQ ID NO:13 or 14), position 619 in ZmoSHC2 (SEQ ID NO:15), position 609 in TeISHC (SEQ ID NO:23) and position 629 in ScoSH1 (SEQ ID NO:32).

In an embodiment, the SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and wherein the SHC/HAC enzyme variant amino acid sequence has amino acid alterations relative to SEQ ID NO: 1, 2, 3, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382, or 383 at positions corresponding to positions W169, A306 and/or G600 of SEQ ID NO: 1, 2, 3 or at a position in an amino acid sequence of a wild type SHC/HAC enzyme corresponding to W169, A306 and/or G600 of SEQ ID NO:1, 2, or 3.

The SHC/HAC enzyme variant amino acid sequence has amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and/or G600 of SEQ ID NO: 1, 2, 3 or at a position in an amino acid sequence of a wild type SHC/HAC enzyme corresponding to W169, A306 and/or G600 of SEQ ID NO:1, 2, or 3.

In an embodiment, the SHC/HAC enzyme or enzyme variant (also named type 1 variants) has an amino acid sequence with at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity to SEQ ID NO: 1, SEQ ID NO:2, SEQ ID NO:3, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO:21, SEQ ID NO:22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO:30, SEQ ID NO:31, SEQ ID NO: 32, SEQ ID NO: 33, SEQ ID NO: 34, SEQ ID NO: 47, SEQ ID NO: 48, SEQ ID NO: 49, SEQ ID NO: 50, SEQ ID NO:51, SEQ ID NO:52, SEQ ID NO: 53, SEQ ID NO: 54 and/or SEQ ID NO: 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307,308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, or 3 at positions corresponding to positions W169, A306 and/or G600 of SEQ ID NO: 1, 2, 3, 47, 48, 49, 50, 51, 52, 53, and/or 54 or at a position in an amino acid sequence of a wild type SHC/HAC enzyme as SEQ ID NO: 13, SEQ ID NO: 14, SEQ ID NO: 15, SEQ ID NO: 16, SEQ ID NO: 17, SEQ ID NO:18, SEQ ID NO: 19, SEQ ID NO: 20, SEQ ID NO:21, SEQ ID NO:22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO:30, SEQ ID NO:31, SEQ ID NO: 32, SEQ ID NO: 33, SEQ ID NO: 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307,308, 309 or 310, corresponding to W169, A306 and/or G600 of SEQ ID NO:1, 2, 3, 47, 48, 49, 50, 51, 52, 53, and/or 54.

In a preferred embodiment, the SHC/HAC enzyme or enzyme variant has an amino acid sequence with at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity to SEQ ID NO: 1, SEQ ID NO:2, SEQ ID NO:3, and has amino acid alterations relative to SEQ ID NO: 1, 2, 3 at positions corresponding to positions W169, A306 and/or G600 of SEQ ID NO: 1, 2, 3.

In an embodiment, a SHC/HAC enzyme variant (also named type 2 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169 of SEQ ID NO: 1, 2, 3 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 47, 48, 49, 50, 51, 52, 53 or 54 corresponding to W169 of SEQ ID NO:1, 2, 3.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant (also named type 3 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54. 361, 362, 363, 364, 365, 366, 367, 368, 369, 370, 371, 372, 373, 374, 375, 376, 377, 378, 379, 380, 381, 382 or 383 at positions corresponding to positions W169 and G600M of SEQ ID NO: 1, 2, 3 or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295,296, 307, 308, 309 or 310 corresponding to W169 and G600M of SEQ ID NO:1, 2, 3.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant (also named type 4 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and G600 of SEQ ID NO: 1, 2, or 3 or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295,296, 307, 308, 309 or 310 corresponding to W169, A306 and G600 of SEQ ID NO:1, 2, or 3.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

All combinations of altered positions in the SHC/AHC enzyme variant identified above are encompassed by the present disclosure:
W169
W169, A306
W169, A306, G600
W169, G600
A306, G600
A306
G600
   (when referring to SEQ ID NO:1)

The new amino acid (X) at a position corresponding to position 169 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 may be G, A or V or a functional equivalent thereof.

A functional equivalent of G is P. A and V are functional equivalents. M, L and I are also functional equivalents of A and V. A preferred new amino acid at position 169 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to 169 of SEQ ID NO:1, 2, or 3 is G, A or V. A most preferred new amino acid at position 169 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to 169 of SEQ ID NO:1, 2, or 3 is G.

For example, position 169 in AacSHC represented by SEQ ID NO:1 corresponds to position 222 in ZmoSHC1 (SEQ ID NO:13), position 177 in ZmoSHC2 (SEQ ID NO:15), position 172 in TeISHC (SEQ ID NO:23) and position 196 in ScoSH1 (SEQ ID NO:32).

The new amino acid (X) at a position corresponding to position 306 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 may be V or a functional equivalent thereof. A functional equivalent of V is A, M, L and I. A preferred new amino acid at position 306 of SEQ ID NO:1, 2, 3, 47, 48, 49, 50, 51, 52, 53, 54 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to 306 of SEQ ID NO:1, 2, or 3 is V.

For example, position 306 in AacSHC represented by SEQ ID NO:1 corresponds to position 368 in ZmoSHC1 (SEQ ID NO:13), position 321 in ZmoSHC2 (SEQ ID NO:15), position 311 in TeISHC (SEQ ID NO:23) and position 335 in ScoSH1 (SEQ ID NO:32).

The new amino acid (X) at a position corresponding to position 600 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 may be A, V, L, I or M or a functional equivalent thereof. All these amino acids are functional equivalents. A preferred new amino acid at position 600 of SEQ ID NO:1, 2, 3, 47, 48, 49, 50, 51, 52, 53, 54 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to 600 of SEQ ID NO:1, 2, 3 is A, V, L, I, or M. A most preferred new amino acid at position 600 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to 600 of SEQ ID NO:1, 2, or 3 is M.

For example, position 600 in AacSHC represented by SEQ ID NO:1 corresponds to position 667 in ZmoSHC1 (SEQ ID NO:13), position 619 in ZmoSHC2 (SEQ ID NO:15), position 609 in TeISHC (SEQ ID NO:23) and position 629 in ScoSH1 (SEQ ID NO:32).

TABLE 9
Positions of the three preferred mutated amino
acids (AAs) in four different wild type SHC/HAC

| | |
|---|---|
| AA position | Corresponding AA position in |

| in AacSHC | ZmoSHC1 | ZmoSHC2 | TelSHC | ScoSHC |
|---|---|---|---|---|
| 169 | 222 | 177 | 172 | 196 |
| 306 | 368 | 321 | 311 | 335 |
| 600 | 667 | 619 | 609 | 629 |

In an embodiment, a SHC/HAC enzyme variant (also named type 5 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and/or G600 of SEQ ID NO: 1, 2, or 3 or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to W169, A306 and/or G600 of SEQ ID NO:1, 2, or 3 and such amino acid alterations are:
the W at position 169 is replaced by G, A or V or a functional equivalent thereof, preferably wherein the W at position 169 is replaced by G,
the A at position 306 is replaced by V or a functional equivalent thereof and/or
the G at position 600 is replaced by A, V, L, I or M or a functional equivalent thereof, preferably wherein the G at position 600 is replace by M.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant (also named type 6 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 and has an amino acid alteration relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at position corresponding to position W169 of SEQ ID NO: 1, 2, or 3 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to W169 of SEQ ID NO:1, 2, or 3 and such amino acid alteration is the W at position 169 is replaced by G.

In an embodiment, the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant (also named type 7 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169 and G600M of SEQ ID NO: 1, 2, 3 or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to W169 and G600M of SEQ ID NO:1, 2, or 3 and such amino acid alterations are:
the W at position 169 is replaced by G and
the G at position 600 is replaced by M.

In an embodiment, the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, and the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by M.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169 and A306 of SEQ ID NO: 1, 2, 3 or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to W169 and A306 of SEQ ID NO:1, 2, or 3 and such amino acid alterations are:
the W at position 169 is replaced by G and
the A at position 306 is replaced by V.

In an embodiment, the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, and
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant (also named type 8 variants) has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and G600 of SEQ ID NO: 1, 2, 3 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to W169, A306 and G600 of SEQ ID NO:1, 2, or 3 and such amino acid alterations are:
the W at position 169 is replaced by G,
the A at position 306 is replaced by V and
the G at position 600 is replaced by M.

In an embodiment, the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G,
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V, and
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by M.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant has an amino acid sequence having at least 70.0% identity or similarity to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169 and G600 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and such amino acid alterations are:
the W at position 169 is replaced by G, and
the G at position 600 is replaced by M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant has an amino acid sequence having at least 70.0% identity or similarity to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and
has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169 and A306 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and such amino acid alterations are:
   the W at position 169 is replaced by G, and
   the A at position 306 is replaced by V.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a SHC/HAC enzyme variant (also named type 9 variants) has an amino acid sequence having at least 70.0% identity or similarity to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and/or G600 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and such amino acid alterations are:
the W at position 169 is replaced by G,
the A at position 306 is replaced by V and
the G at position 600 is replaced by M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (also named type 10 variants) has an amino acid sequence having at least 70.0% identity or similarity to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 305 or 306 and has amino acid alteration relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 305 or 306 at position W169 of SEQ ID NO: 1, 2, 3, 47, 48, 49, 50, 51, 52, 53 or 54 and such amino acid alteration is the W at position 169 is replaced by G.

In an embodiment, each of the SHC/HAC enzyme variant (type 10 variant) having at least 70.0% identity or similarity with SEQ ID NO: 2, 3, 47, 48, 49, 50, 51, 53, 54, 54, 361, 362, 363, 364, 365, 366, 367, 368, 369, 370, 371, 372, 373, 374, 375, 376, 377, 378, 379, 380, 381, 382, 383, 305 or 306 still has the same mutation(s) that differentiates it from SEQ ID NO:1. Each of these mutations has already been defined herein at least in the section entitled “Summary of the sequences”. In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ZmoSHC1 P1 variant, SEQ ID NO: 312) (also named type 10 variants) has an amino acid sequence having at least 70.0% identity or similarity to SEQ ID NO: 13 or 14 and has amino acid alteration relative to SEQ ID NO: 13 at position W222 of SEQ ID NO: 13 or 14 and such amino acid alteration is the W at position 222 is replaced by G.

In a preferred embodiment, this enzyme variant is represented by an amino acid sequence having at least 70% with SEQ ID NO:312 and that still has W222G.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ZmoSHC1 P2 variant: SEQ ID NO: 353) (also named type 10 variants) has an amino acid sequence having at least 70.0% identity or similarity to SEQ ID NO: 13 and has amino acid alterations relative to SEQ ID NO: 13 at positions W222 and G667 of SEQ ID NO: 13 and such amino acid alteration is the W at position 222 is replaced by G and G at position 667 is replaced by M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant has an amino acid sequence having at least 70.0% identity or similarity to SEQ ID NO: 13 and has amino acid alterations relative to SEQ ID NO: 13 at positions W222 and A368 of SEQ ID NO: 13 and such amino acid alteration is the W at position 222 is replaced by G and A at position 368 is replaced by V.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ZmoSHC1 P3 variant: SEQ ID NO: 354) (also named type 10 variants) has an amino acid sequence having at least 70.0% identity or similarity to SEQ ID NO: 13 and has amino acid alterations relative to SEQ ID NO: 13 at positions W222, A368 and G667 of SEQ ID NO: 13 and such amino acid alteration is the W at position 222 is replaced by G, A at position 368 is replaced by V, and G at position 667 is replaced by M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ZmoSHC2 P1 variant, SEQ ID NO: 313) (also named type 10 variants) has an amino acid sequence having at least 70.0% identity or similarity to SEQ ID NO:15 and has amino acid alteration relative to SEQ ID NO: 15 at position W177of SEQ ID NO: 15 and such amino acid alteration is the W at position 177 is replaced by G.

In a preferred embodiment, this enzyme variant is represented by an amino acid sequence having at least 70% with SEQ ID NO:313 and that still has W177G.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ZmoSHC2 P2 variant, SEQ ID NO: 355) (also named type 10 variants) has an amino acid sequence having at least 70.0% identity or similarity to SEQ ID NO:15 and has amino acid alteration relative to SEQ ID NO: 15 at position W177 and G619 of SEQ ID NO: 15 and such amino acid alteration is the W at position 177 is replaced by G and the G at position 619 is replaced by M.

In a preferred embodiment, this enzyme variant is represented by an amino acid sequence having at least 70% with SEQ ID NO:313 or 355 and that still has W177G and G619M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant has an amino acid sequence having at least 70.0% identity or similarity to SEQ ID NO:15 and has amino acid alteration relative to SEQ ID NO: 15 at positions W177, and 321 of SEQ ID NO: 15 and such amino acid alteration is the W at position 177 is replaced by G and the A at position 321 is replaced by V.

In a preferred embodiment, this enzyme variant is represented by an amino acid sequence having at least 70% with SEQ ID NO:313 and that still has W177G and A321V.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ZmoSHC2 P3 variant, SEQ ID NO: 356) (also named type 10 variants) has an amino acid sequence having at least 70.0% identity or similarity to SEQ ID NO:15 and has amino acid alteration relative to SEQ ID NO: 15 at positions W177, A321 and G619 of SEQ ID NO: 15 and such amino acid alteration is the W at position 177 is replaced by G, A at position 321 is replaced by V, and the G at position 619 is replaced by M.

In a preferred embodiment, this enzyme variant is represented by an amino acid sequence having at least 70% with SEQ ID NO:313 and that still has W177G, A321V and G619M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (Tel SHC variant P1 SEQ ID NO: 304) (also named type 10 variants) has an amino acid sequence having at least 70.0% identity or similarity to SEQ ID NO: 23 and has amino acid alteration relative to SEQ ID NO: 23 at position W172 of SEQ ID NO: 23 and such amino acid alteration is the W at position 172 is replaced by G.

In a preferred embodiment, this enzyme variant is represented by an amino acid sequence having at least 70% with SEQ ID NO:304 and that still has W172G.

In a preferred embodiment, this enzyme variant is represented by an amino acid sequence having at least 70% with SEQ ID NO:302 and that still has W172G.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (Tel SHC variant P2 SEQ ID NO: 359) (also named type 10 variants) has an amino acid sequence having at least 70.0% identity or similarity to SEQ ID NO: 23 and has amino acid alteration relative to SEQ ID NO: 23 at positions W172 and G609 of SEQ ID NO: 23 and such amino acid alteration is the W at position 172 is replaced by G and G at position 609 replaced by M.

In a preferred embodiment, this enzyme variant is represented by an amino acid sequence having at least 70% with SEQ ID NO:359 and that still has W172G and G609M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant has an amino acid sequence having at least 70.0% identity or similarity to SEQ ID NO: 23 and
has amino acid alteration relative to SEQ ID NO: 23 at positions W172 and A311 of SEQ ID NO: 23 and such amino acid alteration is the W at position 172 is replaced by G and A at position 311 is replaced by V.

In a preferred embodiment, this enzyme variant is represented by an amino acid sequence having at least 70% with SEQ ID NO:360 and that still has W172G and A311V.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (Tel SHC variant P3 SEQ ID NO: 360) (also named type 10 variants) has an amino acid sequence having at least 70.0% identity or similarity to SEQ ID NO: 23 and has amino acid alteration relative to SEQ ID NO: 23 at positions W172, A311 and G609 of SEQ ID NO: 23 and such amino acid alteration is the W at position 172 is replaced by G, A at position 311 is replaced by V, and G at position 609 replaced by M.

In a preferred embodiment, this enzyme variant is represented by an amino acid sequence having at least 70% with SEQ ID NO:360 and that still has W172G, A31 1V and G609M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ScoSHC1 P1 variant SEQ ID NO: 311) (also named type 10 variants) has an amino acid sequence having at least 70.0% identity or similarity to SEQ ID NO:32 and has amino acid alteration relative to SEQ ID NO: 32 at position W196 of SEQ ID NO: 32 and such amino acid alteration is the W at position 196 is replaced by G.

In a preferred embodiment, this enzyme variant is represented by an amino acid sequence having at least 70% with SEQ ID NO:311 and that still has W196G.

In a preferred embodiment, a SHC/HAC enzyme variant (ScoSHC1 P2 variant SEQ ID NO: 357) (also named type 10 variants) has an amino acid sequence having at least 70.0% identity or similarity to SEQ ID NO:32 and has amino acid alteration relative to SEQ ID NO: 32 at positions W196 and G629 of SEQ ID NO: 32 and such amino acid alteration is the W at position 196 is replaced by G and the G at position 629 is replaced by M.

In a preferred embodiment, this enzyme variant is represented by an amino acid sequence having at least 70% with SEQ ID NO:311 and that still has W196G and G629M.

In a preferred embodiment, a SHC/HAC enzyme variant has an amino acid sequence having at least 70.0% identity or similarity to SEQ ID NO:32 and has amino acid alteration relative to SEQ ID NO: 32 at positions W196, and A335 of SEQ ID NO: 32 and such amino acid alteration is the W at position 196 is replaced by G and the A at position 335 is replaced by V.

In a preferred embodiment, this enzyme variant is represented by an amino acid sequence having at least 70% with SEQ ID NO:311 and that still has W196G and A335V. In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (ScoSHC1 P3 variant SEQ ID NO: 358) (also named type 10 variants) has an amino acid sequence having at least 70.0% identity or similarity to SEQ ID NO:32 and has amino acid alteration relative to SEQ ID NO: 32 at positions W196, A335 and G629 of SEQ ID NO: 32 and such amino acid alteration is the W at position 196 is replaced by G, A at position 335 is replaced by V, and the G at position 629 is replaced by M.

In a preferred embodiment, this enzyme variant is represented by an amino acid sequence having at least 70% with SEQ ID NO:311 and that still has W196G, A335V and G629M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, the enzyme variant is as follows:
the SHC/HAC enzyme variant has an amino acid sequence having at least 70.0% identity to SEQ ID NO: 1, 4, 5, 6 or 350 and has at least one of the following mutations: W169G, A306V and G600M or
the SHC/HAC enzyme variant has an amino acid sequence having at least 90.0% identity to SEQ ID NO: 7, 8, 9 or 386 and has the following mutations: M132R, A224V, 1432T, A557T, R613S, and has at least one of the following mutations: W169G, A306V and G600M or
the SHC/HAC enzyme variant has an amino acid sequence having at least 90.0% identity to SEQ ID NO: 10, 11, 12 or 385 and has the following mutations: M132R, A224V, 1432T and has at least one of the following mutations: W169G, A306V and G600M or
the SHC/HAC enzyme variant has an amino acid sequence having at least 70.0% identity to SEQ ID NO: 23, 304, 359 or 360 and has at least one of the following mutations: W172G, A311V and G609M (Tel SHC/HAC variant) or
the SHC/HAC enzyme variant has an amino acid sequence having at least 70.0% identity to SEQ ID NO: 32, 311, 357, or 358 and has the following mutations: W196G, A335V and G629M (Sco SHC/HAC variant) or
the SHC/HAC enzyme variant has an amino acid sequence having at least 70.0% identity to SEQ ID NO:13, 312, 353 or 354 and has at least one of the following mutations: W222G, A368V and G667M (Zmo SHC1 variant) or
the SHC/HAC enzyme variant has an amino acid sequence having at least 70.0% identity to SEQ ID NO: 15, 313, 355 or 356 and has at least one of the following mutations: W177G, A321V and G619M (Zmo SHC2 variant) or
the SHC/HAC enzyme variant has an amino acid sequence having at least 70.0% identity to SEQ ID NO: 1 and has at least the following mutations: W169G, G600M, M132R and 1432T, or
the SHC/HAC enzyme variant has an amino acid sequence having at least 90.0% identity to SEQ ID NO: 1 and has the following mutations: W169G, G600M, M132R and 1432T, or
the SHC/HAC enzyme variant has an amino acid sequence having at least 95.0% identity to SEQ ID NO: land has the following mutations W169G, G600M, M132R and 1432T, or
the SHC/HAC enzyme variant has an amino acid sequence having at least 96.0%, 97.0%, 99.0%, 99.0% identity to SEQ ID NO: 1 and has the following mutations W169G, G600M, M132R and 1432T.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a preferred embodiment, a SHC/HAC enzyme variant (also named type 11 variants) has an amino acid sequence having at least 70.0% identity or similarity to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, and G600M of SEQ ID NO: 1, 2, 3, 47, 48, 49, 50, 51, 52, 53 or 54, 361, 362, 363, 364, 365, 366, 367, 368, 369, 370, 371, 372, 373, 374, 375, 376, 377, 378, 379, 380, 381, 382 or 383 and such amino acid alterations are:
the W at position 169 is replaced by G and
the G at position 600 is replaced by M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the SHC/HAC enzyme variant (also named type 12 variants) has an amino acid sequence having at least 70.0% identity to SEQ ID NO: 1 and has at least one of the following mutations relative to SEQ ID NO:1: W169G, A306V and G600M.

In an embodiment, the SHC/HAC enzyme variant (also named type 13 variants) has an amino acid sequence having at least 70.0% identity to SEQ ID NO: 4, and has at least the following mutation relative to SEQ ID NO:1: W169G.

In an embodiment, the SHC/HAC enzyme variant (also named type 14 variants) has an amino acid sequence having at least 70.0% identity to SEQ ID NO: 5, and has at least the following mutations relative to SEQ ID NO:1: W169G and G600M.

In an embodiment, the SHC/HAC enzyme variant has an amino acid sequence having at least 70.0% identity to SEQ ID NO: 350, and has at least the following mutations relative to SEQ ID NO:1: W169G and A306V.

In an embodiment, the SHC/HAC enzyme variant (also named type 15 variants) has an amino acid sequence having at least 70.0% identity to SEQ ID NO: 6, and has at least the following mutationS relative to SEQ ID NO:1: W169G, A306V and G600M.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the SHC/HAC enzyme variant (also named type 16 variants) has an amino acid sequence having at least 70.0% identity to SEQ ID NO: 3, has the following mutations M132R, A224V, 1432T relative to SEQ ID NO: 1 and has at least one of the following mutations: W169G, A306V and G600M relative to SEQ ID NO: 3.

In an embodiment, the SHC/HAC enzyme variant (also named type 17 variants) has an amino acid sequence having at least 70.0% identity to SEQ ID NO: 10, has the following mutations M132R, A224V, 1432T relative to SEQ ID NO: 1 and has the following mutation: W169G relative to SEQ ID NO: 3.

In an embodiment, the SHC/HAC enzyme variant (also named type 18 variants) has an amino acid sequence having at least 70.0% identity to SEQ ID NO: 11, has the following mutations M132R, A224V, 1432T relative to SEQ ID NO: 1 and has the following mutations: W169G and G600M relative to SEQ ID NO: 3.

In an embodiment, the SHC/HAC enzyme variant (also named type 32 variants) has an amino acid sequence having at least 70.0% identity to SEQ ID NO: 385, has the following mutations M132R, A224V, 1432T relative to SEQ ID NO: 1 and has the following mutations: W169G and A306V relative to SEQ ID NO: 3.

In an embodiment, the SHC/HAC enzyme variant (also named type 19 variants) has an amino acid sequence having at least 70.0% identity to SEQ ID NO: 12, has the following mutations M132R, A224V, 1432T relative to SEQ ID NO: 1 and has the following mutations: W169G, A306V and G600M relative to SEQ ID NO: 3.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the SHC/HAC enzyme variant (also named type 20 variants) has an amino acid sequence having at least 70.0% identity to SEQ ID NO: 2, has the following mutations: M132R, A224V, 1432T, A557T, R613S relative to SEQ ID NO: 1 and has at least one of the following mutations: W169G, A306V and G600M relative to SEQ ID NO: 2.

In an embodiment, the SHC/HAC enzyme variant (also named type 21 variants) has an amino acid sequence having at least 70.0% identity to SEQ ID NO: 7, has the following mutations M132R, A224V, 1432T, A557T, R613S relative to SEQ ID NO: 1 and has the following mutation: W169G relative to SEQ ID NO: 2.

In an embodiment, the SHC/HAC enzyme variant (also named type 22 variants) has an amino acid sequence having at least 70.0% identity to SEQ ID NO: 8, has the following mutations M132R, A224V, 1432T, A557T, R613S relative to SEQ ID NO: 1 and has the following mutations: W169G and G600M relative to SEQ ID NO: 2.

In an embodiment, the SHC/HAC enzyme variant (also named type 33 variants) has an amino acid sequence having at least 70.0% identity to SEQ ID NO:386, has the following mutations M132R, A224V, 1432T, A557T, R613S relative to SEQ ID NO: 1 and has the following mutations: W169G and A306V relative to SEQ ID NO: 2.

In an embodiment, the SHC/HAC enzyme variant (also named type 23 variants) has an amino acid sequence having at least 70.0% identity to SEQ ID NO: 9, has the following mutations M132R, A224V, 1432T, A557T, R613S relative to SEQ ID NO: 1 and has the following mutations: W169G, A306V and G600M relative to SEQ ID NO: 2.

In this context, the identity or similarity may be of at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the SHC/AHC enzyme variant (also named type 34 variants) has an amino acid sequence having at least 30.0% identity or similarity to SEQ ID NO: 1-12, 16, 17, 18, 19, 20, 21, 22, 23, 24, 25, 26, 27, 28, 29, 30, 31, 32, 33, 34, 47-54, 55-296, 301-313, 315-353, 354-383 wherein the W at position 169 or corresponding to position 169 of SEQ ID NO: 1, is replaced by G; wherein the A at position 306 or corresponding to position 306 of SEQ ID NO: 1, 2 or 3 is replaced by V; and/or wherein the G at position 600 or corresponding to 600 of SEQ ID NO: 1, 2 or 3 is replaced by M.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the SHC/AHC enzyme variant (also named type 24 variants) has an amino acid sequence comprising or consisting of or essentially consisting of SEQ ID NO: 4, 5, 6, 7, 8, 9, 10, 11, 12, 304, 311, 312, 313, 305, 306, 302, 359, 357, 353, 355, 360, 358, 354, 356.

In an embodiment, the SHC/AHC enzyme variant (also named type 25 variants) has an amino acid sequence comprising or consisting of or essentially consisting of SEQ ID NO: 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 350, 351, 352.

In an embodiment, each of the SHC/HAC enzyme variants defined herein (type 1 to 34 variants) may have the following additional amino acid alteration wherein the (new) amino acid (X) at a position corresponding to position 168 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 may be S or a functional equivalent thereof. A functional equivalent of S is C, T, N or Q. A preferred new amino acid at position 168 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295,296, 307, 308, 309 or 310 corresponding to 168 of SEQ ID NO:1, 2 or 3 is S.

In a preferred embodiment, a SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 and
has an amino acid alteration relative to SEQ ID NO: 1, 2, 3, 47, 48, 49, 50, 51, 52, 53 and/or 54 at position corresponding to position W169 of SEQ ID NO: 1, 2, 3 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 to W169 of SEQ ID NO:1, 2, 3 and such amino acid alteration is the W at position 169 is replaced by G, and
has amino acid S at position 168 relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at position corresponding to position S168 of SEQ ID NO: 1, 2, 3 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to S168 of SEQ ID NO:1, 2, 3.

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, any SHC/HAC enzyme variant may comprise at least one of the following conserved amino acids L22, Q26, G30, W32, A44, L48, Q72, G76, W78, Y95, L98, G102, A113, 1117, G121, G122, F129, T130, L134, A135, G138, W142, P146, W169, A170, R171, F217, D222, R237, 1261, P263, P281, S309, P310, W312, D313, T314, A320, W339, Q344, G349, D350, W351, G361, G362, A364, F365, N369, Y372, P373, D374, D376, D377, W406, Q411, G415, A419, P433, D436, D442, P443, D447, V448, Q479, G483, W485, G487, R488, W489, G490, N492, Y495, G496, T497, L504, W522, Q527, G531, G532, W533, G534, E535, S539, Y540, G547, T552, T556, W558, A559, A565, L581, Q585, G589, W591, G600, F601, P602, F605, Y609, Y612, F616, P617, A620 and R623 by reference to the wild type Aac SHC/HAC having SEQ ID NO: 1 or any variant derived from SEQ ID NO:1 and having SEQ ID NO: 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383. This variant may also be derived from any wild type SHC/HAC enzyme as defined herein (e.g. SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310) wherein corresponding positions are identified corresponding to the ones identified above for SEQ ID NO: 1. This variant may be derived from any variant types defined earlier herein.

There is therefore provided herein a process for making (−)-Ambrox by enzymatically converting EEH to (−)-Ambrox. There is also provided herein a process for making Ambra oxide by enzymatically converting E,E-bishomofarnesol to Ambra oxide. These processes may use any SHC/HAC enzyme variant described herein (especially of types 1 to 25).

“Percent (%) identity” with respect to a polypeptide or nucleotide sequence is defined respectively as the percentage of amino acids or nucleotides in a candidate sequence that are identical with the amino acids or nucleotides in the reference sequence, after aligning the sequence and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared. The terms “polypeptide” and “protein” are used interchangeably herein and mean any peptide-linked chain of amino acids, regardless of length or post-translational modification.

The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available eg. on https://www.ebi.ac.uk/Tools/msa/clustalo/or the GAP program (mathematical algorithm of the University of Iowa) or the mathematical algorithm of Myers and Miller (1989—Cabios 4: 11-17). Preferred parameters used are the default parameters as they are set on https://www.ebi.ac.uk/Tools/msa/clustalo/.

Percentage sequence identity may be calculated using, for example, BLAST, BLAT or BlastZ (or BlastX). A similar algorithm is incorporated into the BLASTN and BLASTP programs of Altschul et al (1990) J. Mol. Biol. 215, 403-410. BLAST polynucleotide searches may be performed with the BLASTN program, score=100, word length=12, to obtain polynucleotide sequences that are homologous to those nucleic acids which encode the relevant protein. BLAST protein searches may be performed with the BLASTP program, score=50, word length=3, to obtain amino acid sequences homologous to the polypeptide.

To obtain gapped alignments for comparative purposes, Gapped BLAST may be utilized as described in Altschul et al (1997) Nucleic Acids Res. 25, 3389-3402. When utilizing BLAST and Gapped BLAST programs, the default parameters of the respective programs are used.

Sequence matching analysis may be supplemented by established homology mapping techniques like Shuffle-LAGAN (Brudno M., Bioinformatics 2003b, 19 Suppl 1: 154-162) or Markov random fields. When percentages of sequence identity are referred to in the present application, these percentages are calculated in relation to the full length of the longer sequence, if not specifically indicated otherwise.

In particular embodiments, % identity between two sequences is determined using CLUSTAL O (version 1.2.4).

Additional amino acid alterations may be present in the SHC/AHC variants disclosed herein. In an embodiment, examples of additional amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 and are at positions corresponding to positions 81, 431, 90, 172, 277, 37, 174, 601, 77, 92, 129, 579, 605 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 5556, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to 81, 431, 90, 272, 177, 37, 174, 601, 77, 92, 129, 579, 605 of SEQ ID NO:1, 2, or 3.

Variant of type 26: In an embodiment, examples of additional amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382, 383 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 and are at positions corresponding to positions 81 and/or 431 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to 81, 431 of SEQ ID NO:1, 2, or 3.

Variant of type 27: In an embodiment, examples of additional amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 and are at positions corresponding to positions 90, 172 and/or 277 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to 90, 272, 177 of SEQ ID NO:1, 2, or 3.

Variant of type 28: In an embodiment, examples of additional amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 and are at positions corresponding to positions 37, 174, 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 corresponding to 37, 174, 601 of SEQ ID NO:1, 2, or 3.

Variant of type 29: In an embodiment, examples of additional amino acid alterations relative to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 are at positions corresponding to positions 77, 92, 129, 579, 601, 605 of SEQ ID NO: 1, 2, 3, 47, 48, 49, 50, 51, 52, 53 and/or 54 or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to 77, 92, 129, 579, 601, 605 of SEQ ID NO:1, 2, or 3.

The amino acid alteration at a position corresponding to position 81 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20 21, 22, 23, 24, 25, 26, 27, 28, 29, 30, 31, 32, 33, 34, 55-296, 307-310 corresponding to 81 of SEQ ID NO:1, 2, or 3) may, for example, be Y81X. This refers to a substitution of the amino acid Y at position 81 of SEQ ID NO: 1 for any different amino acid (X). As noted above, since the SHC/HAC enzyme variants may additionally comprise insertions and/or deletions, the numbering of the new amino acid (X) in the new SHC/HAC enzyme variant may not be 81.

The new amino acid (X) at a position corresponding to position 81 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 81 of SEQ ID NO:1, 2, 3) may, for example, be Met, Ala, Val, Leu, lie, Cys, Ser, Thr, Asn, Gln, Asp, Glu, His, Lys, Arg, Gly, Pro, Trp, or Phe. For example, the amino acid alteration at a position corresponding to position 81 of SEQ ID NO: 1 may substitute the amino acid of SEQ ID NO: 1 (i.e. Y) for a basic amino acid (i.e. His, Lys or Arg). For example, the amino acid alteration at a position corresponding to position 81 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 81 of SEQ ID NO:1, 2, or 3) may substitute the amino acid of SEQ ID NO: 1 (i.e. Y) for histidine (i.e. the amino acid alteration at a position corresponding to position 81 of SEQ ID NO: 1 is Y81H).

The amino acid alteration at a position corresponding to position 431 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 431 of SEQ ID NO:1, 2, 3) may, for example, be H431X. This refers to a substitution of the amino acid H at position 431 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 81 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 431.

The new amino acid (X) at a position corresponding to position 431 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 81 of SEQ ID NO:1, 2, 3) may, for example, be Met, Ala, Val, Leu, lie, Cys, Ser, Thr, Asn, Gln, Asp, Glu, Lys, Arg, Gly, Pro, Trp, Tyr or Phe. For example, the amino acid alteration at a position corresponding to position 431 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55-296, 307-310 corresponding to 81 of SEQ ID NO:1, 2, or 3) may substitute the amino acid of SEQ ID NO: 1 (i.e. H) for a hydrophobic amino acid (i.e. Met, Ala, Val, Leu or lie). For example, the amino acid alteration at a position corresponding to position 431 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 81 of SEQ ID NO:1, 2, 3) may substitute the amino acid of SEQ ID NO: 1 (i.e. H) for leucine (i.e. the amino acid alteration at a position corresponding to position 431 of SEQ ID NO: 1 is H431L).

The amino acid alteration at a position corresponding to position 90 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 90 of SEQ ID NO:1, 2, or 3) may, for example, be T90X. This refers to a substitution of the amino acid T at position 90 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 90 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 90.

The new amino acid (X) at a position corresponding to position 90 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 90 of SEQ ID NO:1, 2, 3) may, for example, be Met, Ala, Val, Leu, lie, Cys, Ser, Asn, Gln, Asp, Glu, His, Lys, Arg, Gly, Pro, Trp, Tyr or Phe. For example, the amino acid alteration at a position corresponding to position 90 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 90 of SEQ ID NO:1, 2, or 3) may substitute the amino acid of SEQ ID NO: 1 (i.e. T) for a hydrophobic amino acid (i.e. Met, Ala, Val, Leu, lie). For example, the amino acid alteration at a position corresponding to position 90 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 90 of SEQ ID NO:1, 2, or 3) may substitute the amino acid of SEQ ID NO: 1 (i.e. T) for alanine (i.e. the amino acid alteration at a position corresponding to position 90 of SEQ ID NO: 1 is T90A).

The amino acid alteration at a position corresponding to position 172 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 172 of SEQ ID NO:1, 2, 3) may, for example, be A172X. This refers to a substitution of the amino acid T at position 172 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 172 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 172.

The new amino acid (X) at a position corresponding to position 172 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 172 of SEQ ID NO:1, 2, or 3) may, for example, be Met, Val, Leu, lie, Cys, Ser, Thr, Asn, Gln, Asp, Glu, His, Lys, Arg, Gly, Pro, Trp, Tyr or Phe. For example, the amino acid alteration at a position corresponding to position 172 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 172 of SEQ ID NO:1, 2, or 3) may substitute the amino acid of SEQ ID NO: 1 (i.e. A) for a neutral hydrophilic amino acid (i.e. Cys, Ser, Thr, Asn, Gln). For example, the amino acid alteration at a position corresponding to position 172 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 172 of SEQ ID NO:1, 2, or 3) may substitute the amino acid of SEQ ID NO: 1 (i.e. A) for threonine (i.e. the amino acid alteration at a position corresponding to position 172 of SEQ ID NO: 1 is A172T).

The amino acid alteration at a position corresponding to position 277 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 277 of SEQ ID NO:1, 2, or 3) may, for example, be M277X. This refers to a substitution of the amino acid M at position 277 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 277 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 277.

The new amino acid (X) at a position corresponding to position 277 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 81 of SEQ ID NO:1, 2, or 3) may, for example, be Ala, Val, Leu, lie, Cys, Ser, Thr, Asn, Gln, Asp, Glu, His, Lys, Arg, Gly, Pro, Trp, Tyr or Phe. For example, the amino acid alteration at a position corresponding to position 277 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19 or 20, 21, 22, 23, 24, 25, 26, 27, 28, 29, 30, 31, 32, 33, 34, 55-296, 307-310 corresponding to 277 of SEQ ID NO:1, 2, or 3) may substitute the amino acid of SEQ ID NO: 1 (i.e. M) for a basic amino acid (i.e. His, Lys, Arg). For example, the amino acid alteration at a position corresponding to position 277 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 277 of SEQ ID NO:1, 2, or 3) may substitute the amino acid of SEQ ID NO: 1 (i.e. M) for lysine i.e. the amino acid alteration at a position corresponding to position 277 of SEQ ID NO: 1 is M277K).

The amino acid alteration at a position corresponding to position 37 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 37 of SEQ ID NO:1, 2, or 3) may, for example, be L37X. This refers to a substitution of the amino acid L at position 37 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 37 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 37.

The new amino acid (X) at a position corresponding to position 37 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 37 of SEQ ID NO:1, 2, or 3) may, for example, be Met, Ala, Val, lie, Cys, Ser, Thr, Asn, Gln, Asp, Glu, His, Lys, Arg, Gly, Pro, Trp, Tyr or Phe. For example, the amino acid alteration at a position corresponding to position 37 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 37 of SEQ ID NO:1, 2, or 3) may substitute the amino acid of SEQ ID NO: 1 (i.e. L) for a neutral hydrophilic amino acid (i.e. Cys, Ser, Thr, Asn or Gln). For example, the amino acid alteration at a position corresponding to position 37 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 37 of SEQ ID NO:1, 2, or 3) may substitute the amino acid of SEQ ID NO: 1 (i.e. L) for glutamine (i.e. the amino acid alteration at a position corresponding to position 37 of SEQ ID NO: 1 is L37Q).

The amino acid alteration at a position corresponding to position 174 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 174 of SEQ ID NO:1, 2, or 3) may, for example, be V174X. This refers to a substitution of the amino acid V at position 174 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 277 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 174.

The new amino acid (X) at a position corresponding to position 174 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 174 of SEQ ID NO:1, 2, or 3) may, for example, be Met, Ala, Leu, lie, Cys, Ser, Thr, Asn, Gln, Asp, Glu, His, Lys, Arg, Gly, Pro, Trp, Tyr or Phe. The new amino acid (X) at a position corresponding to position 174 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 174 of SEQ ID NO:1, 2, or 3) may, for example, be a hydrophobic amino acid (i.e. Met, Ala, Leu or lie).

For example, the amino acid alteration at a position corresponding to position 174 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 174 of SEQ ID NO:1, 2, 3) may substitute the amino acid of SEQ ID NO: 1 (i.e. V) for isoleucine (i.e. the amino acid alteration at a position corresponding to position 174 of SEQ ID NO: 1 is V1741).

The amino acid alteration at a position corresponding to position 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 601 of SEQ ID NO:1, 2, or 3) may, for example, be F601X. This refers to a substitution of the amino acid F at position 601 of SEQ ID NO: 1 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 601 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 601.

The new amino acid (X) at a position corresponding to position 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 2, 21, 22, 23, 24, 25, 26, 27, 28, 29, 30, 31, 32, 33, 34, 55-296, 307-310 corresponding to 601 of SEQ ID NO:1, 2, or 3) may, for example, be Met, Ala, Val, Leu, lie, Cys, Ser, Thr, Asn, Gln, Asp, Glu, His, Lys, Arg, Gly, Pro, Trp or Tyr. The new amino acid (X) at a position corresponding to position 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 601 of SEQ ID NO:1, 2, or 3) may, for example, be an aromatic acid (i.e. Trp, Tyr, Phe). For example, the amino acid alteration at a position corresponding to position 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 601 of SEQ ID NO:1, 2, or 3) may substitute the amino acid of SEQ ID NO: 1 (i.e. F) for tyrosine (i.e. the amino acid alteration at a position corresponding to position 601 of SEQ ID NO: 1 is F601Y).

The amino acid alteration at a position corresponding to position 77 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 77 of SEQ ID NO:1, 2, or 3) may, for example, be T77X. This refers to a substitution of the amino acid T at position 77 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 77 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 77.

The new amino acid (X) at a position corresponding to position 77 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 77 of SEQ ID NO:1, 2, or 3) may, for example, be Met, Ala, Val, Leu, lie, Cys, Ser, Asn, Gln, Asp, Glu, His, Lys, Arg, Gly, Pro, Trp, Tyr or Phe. For example, the amino acid alteration at a position corresponding to position 77 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 77 of SEQ ID NO:1, 2, or 3) may substitute the amino acid of SEQ ID NO: 1 (i.e. T) for a hydrophobic amino acid (i.e. Met, Ala, Val, Leu or lie). For example, the amino acid alteration at a position corresponding to position 77 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 77 of SEQ ID NO:1, 2, or 3) may substitute the amino acid of SEQ ID NO: 1 (i.e. T) for alanine (i.e. the amino acid alteration at a position corresponding to position 77 of SEQ ID NO: 1 is T77A).

The amino acid alteration at a position corresponding to position 92 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 92 of SEQ ID NO:1, 2, 3) may, for example, be 192X. This refers to a substitution of the amino acid I at position 92 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 92 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 92.

The new amino acid (X) at a position corresponding to position 92 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 92 of SEQ ID NO:1, 2, or 3) may, for example, be Met, Ala, Val, Leu, Cys, Ser, Thr, Asn, Gln, Asp, Glu, His, Lys, Arg, Gly, Pro, Trp, Tyr or Phe. For example, the amino acid alteration at a position corresponding to position 92 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 92 of SEQ ID NO:1, 2, or 3) may substitute the amino acid of SEQ ID NO: 1 (i.e. 1) for a hydrophobic amino acid (i.e. Met, Ala, Val, Leu or lie). For example, the amino acid alteration at a position corresponding to position 92 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 92 of SEQ ID NO:1, 2, 3) may substitute the amino acid of SEQ ID NO: 1 (i.e. 1) for valine (i.e. the amino acid alteration at a position corresponding to position 92 of SEQ ID NO: 1 is 192V).

The amino acid alteration at a position corresponding to position 129 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 129 of SEQ ID NO:1, 2, or 3) may, for example, be F129X. This refers to a substitution of the amino acid F at position 129 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 129 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 129.

The new amino acid (X) at a position corresponding to position 129 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 129 of SEQ ID NO:1, 2, or 3) may, for example, be Met, Ala, Val, Leu, lie, Cys, Ser, Thr, Asn, Gln, Asp, Glu, His, Lys, Arg, Gly, Pro, Trp or Tyr. For example, the amino acid alteration at a position corresponding to position 129 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 129 of SEQ ID NO:1, 2, or 3) may substitute the amino acid of SEQ ID NO: 1 (i.e. F) for a hydrophobic amino acid (i.e. Met, Ala, Val, Leu or lie). For example, the amino acid alteration at a position corresponding to position 129 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 129 of SEQ ID NO:1, 2, or 3) may substitute the amino acid of SEQ ID NO: 1 (i.e. F) for leucine (i.e. the amino acid alteration at a position corresponding to position 129 of SEQ ID NO: 1 is F129L).

The amino acid alteration at a position corresponding to position 579 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383(or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 579 of SEQ ID NO:1, 2, or 3) may, for example, be Q579X. This refers to a substitution of the amino acid Q at position 579 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 579 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 579.

The new amino acid (X) at a position corresponding to position 579 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 579 of SEQ ID NO:1, 2, or 3) may, for example, be Met, Ala, Val, Leu, lie, Cys, Ser, Thr, Asn, Asp, Glu, His, Lys, Arg, Gly, Pro, Trp, Tyr or Phe. For example, the amino acid alteration at a position corresponding to position 579 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 579 of SEQ ID NO:1, 2, or 3) may substitute the amino acid of SEQ ID NO: 1 (i.e. Q) for a basic amino acid (i.e. His, Lys or Arg). For example, the amino acid alteration at a position corresponding to position 579 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 579 of SEQ ID NO:1, 2, or 3) may substitute the amino acid of SEQ ID NO: 1 (i.e. Q) for histidine (i.e. the amino acid alteration at a position corresponding to position 579 of SEQ ID NO: 1 is Q579H).

The amino acid alteration at a position corresponding to position 605 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 605 of SEQ ID NO:1, 2, or 3) may, for example, be F605X. This refers to a substitution of the amino acid F at position 605 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 605 of SEQ ID NO:1, 2, or 3) for any different amino acid (X). As noted above, since the SHC/HAC enzyme variants may additionally comprise insertions and/or deletions, the numbering of the new amino acid (X) in the new SHC/HAC enzyme variant may not be 605.

The new amino acid (X) at a position corresponding to position 605 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 605 of SEQ ID NO:1, 2, or 3) may, for example, be Met, Ala, Val, Leu, lie, Cys, Ser, Thr, Asn, Gln, Asp, Glu, His, Lys, Arg, Gly, Pro, Trp or Tyr. The new amino acid (X) at a position corresponding to position 605 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 605 of SEQ ID NO:1, 2, or 3) may, for example, be an aromatic acid (i.e. Trp, Tyr, Phe). For example, the amino acid alteration at a position corresponding to position 605 of SEQ ID NO: 1, 2, or 3 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 605 of SEQ ID NO:1, 2, or 3) may substitute the amino acid of SEQ ID NO: 1 (i.e. F) for tryptophan (i.e. the amino acid alteration at a position corresponding to position 601 of SEQ ID NO: 1 is F605W).

For example, the SHC/HAC enzyme variant may have amino acid alterations (e.g. substitutions) at positions corresponding to positions 169, 306, 600, 132 and 432 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20 21, 22, 23, 24, 25, 26, 27, 28, 29, 30, 31, 32, 33, 34, 55-296, 307-310 corresponding to 169, 306, 600, 132 and 432 of SEQ ID NO:1, 2, or 3).

For example, the SHC/HAC enzyme variant is as earlier defined herein and may have an amino acid alteration (e.g. substitution) at a position corresponding to position 169, 306, 600, 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 601 of SEQ ID NO:1, 2, or 3).

For example, the SHC/HAC enzyme variant is as earlier defined herein and may have amino acid alterations (e.g. substitutions) at positions corresponding to positions 169, 306, 600, 77, 92 and 129 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 77, 92, 129 of SEQ ID NO:1, 2, or 3).

For example, the SHC/HAC enzyme variant is as earlier defined herein and may have amino acid alterations (e.g. substitutions) at positions corresponding to positions 169, 306, 600, 579 and 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33.34, 55-296, 307-310 corresponding to 169, 306, 600, 579 and 601 of SEQ ID NO:1, 2, or 3).

For example, the SHC/HAC enzyme variant is as earlier defined herein and may have amino acid alterations (e.g. substitutions) at positions corresponding to positions 169, 306, 600, 129, 132 and 432 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 129, 132, 432 of SEQ ID NO:1, 2, or 3).

For example, the SHC/HAC enzyme variant is as earlier defined herein and may have amino acid alterations (e.g. substitutions) at positions corresponding to positions 169, 306, 600, 132, 432 and 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 132, 432, 601 of SEQ ID NO:1, 2, or 3).

For example, the SHC/HAC enzyme variant is as earlier defined herein and may have amino acid alterations (e.g. substitutions) at positions corresponding to positions 169, 306, 600, 129, 132, 432 and 601 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 129, 132, 432, 601 of SEQ ID NO:1, 2, or 3).

The new SHC/HAC enzyme variants may, for example, exhibit attractive biological activities as earlier defined herein. The new enzyme variants may exhibit increased enzymatic activity for the conversion of EEH to (−)-Ambrox or the conversion of BisEEH to Ambra oxide compared to the SHC/HAC enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, or 307-310. Increased enzymatic activity may refer to any aspect of the enzymatic conversion of EEH to (−)-Ambrox or enzymatic conversion of BisEEH to Ambra oxide including, for example, increased total conversion of EEH or BisEEH, increased rate of conversion of EEH or BisEEH (e.g. in the first 3 hours or in the first 4 hours of reaction or in the first 5 hours of reaction), increased production of (−)-Ambrox or Ambra oxide, and decreased production of by-products. Increased enzymatic activity may be defined by increased productivity in general, which may be defined in terms of (−)-Ambrox or Ambra oxide produced per gram of biocatalyst, per hour and per liter of reaction.

In an embodiment, the SHC/HAC enzyme variant disclosed herein exhibits:
an increased substrate specificity for EEH (or for BisEEH) when homofarnesol or bis-homofarnesol substrates are used,
an increased product selectivity for (−)-Ambrox (or Ambra oxide) when homofarnesol or bis-homofarnesol substrates are used,
an increased specificity for a particular isomer of substrates when substrates other than homofarnesol or bis-homofarnesol are used (eg ethyl-homofarnesol, hydroxyfarnesylacetone and the like—see, for example WO2021/110858 and WO2021/209482 respectively)
an increased productivity and/or
an increased degree of conversion of EEH (or BisEEH) as well as an increased conversion rate of EEH (or BisEEH) over the first 3 to 6 hours (or over the first 4, 5, or 6 hours) of the reaction,
   compared to the reference SHC enzyme (e.g. wild-type SHC such as those represented by SEQ ID NO:1, 13, 14, 15, 16, 17, 18, 19, 20, 21, 22, 23, 24, 25, 26, 27, 28, 29, 30, 31, 32, 33, 34, 55-296, 307-310 or 215G2 AacSHC or SHC #65 or a parent SHC enzyme the variant derives from such as those represented by SEQ ID NO:2, 3, 47-54).

The new SHC/HAC enzyme variants as defined herein (variants of all types 1-34) may, for example, provide increased EEH or E,E-bishomofarnesol (BisEEH) conversion compared to the SHC/HAC wild-type enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, 307-310. Therefore, the process described herein may have an increased level of EEH or BisEEH conversion compared to the process using the SHC/HAC wild-type enzyme of SEQ ID NO:1, 2, 3, 13, 14, 15, 16, 17, 18, 19, 20, 21, 22, 23, 24, 25, 26, 27, 28, 29, 30, 31, 32, 33, 34, 55-296, 307-310. The new SHC/HAC enzyme variants may, for example, provide increased rate of EEH or BisEEH conversion compared to the SHC/HAC wild-type enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, 307-310. Therefore, the process described herein may have an increased rate of EEH or BisEEH conversion compared to the SHC/HAC wild-type enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, 307-310. The new SHC/HAC enzyme variants as defined herein (variants of all types 1-34) may, for example, provide increased rate of EEH or BisEEH conversion over the first 4 hours or over the first 3 hours or over the first 4 hours or over the first 5 hours or over the first 6 hours of the reaction compared to the SHC/HAC wild-type enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, 307-310.

Therefore, the process described herein may have an increased rate of EEH or BisEEH conversion over the first 3 hours or over the first 4 hours or over the first 5 hours or over the first 6 hours compared to the SHC/HAC wild-type enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, 307-310. This may be assessed using the new enzyme versus the reference enzyme under the same reaction conditions (e.g. same pH and temperature) or using each enzyme under its respective optimized reaction conditions (e.g. optimized pH and temperature) which may be different to each other.

For example, the new SHC/HAC enzyme variant as defined herein (variants of all types 1-34) may provide or the process may have at least about 40% EEH or BisEEH conversion in the first 6 hours of the reaction. For example, the new SHC/HAC enzyme variant may provide or the process may have at least about 45% or at least about 50% or at least about 55% or at least about 60% EEH or BisEEH conversion in the first 6 hours of the reaction. For example, the new SHC/HAC enzyme variant may provide or the process may have at least about 30% EEH or BisEEH conversion in the first 5 hours of the reaction. For example, the new SHC/HAC enzyme variant may provide or the process may have at least about 35% or at least about 45% or at least about 50% or at least about 55% EEH or BisEEH conversion in the first 6 hours of the reaction. This may be when compared to using both enzymes (i.e. the new SHC/HAC enzyme variant and the enzyme of SEQ ID NO: 1, 2, 3, 13, 14, 15, 16, 17, 18, 19, 20, 21, 22, 23, 24, 25, 26, 27, 28, 29, 30, 31, 32, 33, 34, 55-296, 307-310) under the same reaction conditions (e.g. same pH and temperature) or when compared to using each enzyme under its respective optimized reaction conditions (e.g. optimized pH and temperature) which may be different to each other.

The conversion of EEH to (−)-Ambrox or the conversion of BisEEH to Ambra oxide or the conversion of hydroxyfarnesylacetone to Amberketal (see for example, WO2021/209482) may, for example, be determined using an activity assay as described above and may be calculated as gram of recoverable product per gram of feedstock (which can be calculated as a percent molar conversion rate).

As used herein, any reference herein to a 99%/100% conversion rate for a homofarnesol substrate to (−)-Ambrox is a reference to a 99%/100% conversion of the homofarnesol isomer (i.e. EEH) capable of conversion to (−)-Ambrox using a SHC/HAC enzyme or enzyme variant.

As used herein, any reference herein to a 99%/100% conversion rate for a bishomofarnesol substrate to Ambra oxide is a reference to a 99%/100% conversion of the bishomofarnesol isomer (i.e. BisEEH) capable of conversion to Ambra oxide using a SHC/HAC enzyme or enzyme variant.

As used herein, any reference herein to a 99%/100% conversion rate for a hydroxyfarnesyl substrate to Amberketal is a reference to a 99%/100% conversion of the hydroxyfarnesyl substrate capable of conversion to Amberketal using a SHC/HAC enzyme or enzyme variant. The production of Amberketal from a hydroxyfarnesylacetone substrate using a WT SHC enzyme or an SHC variant is disclosed in WO2021/209482, the contents of which is incorporated herein by reference.

The optimum temperature range for the wild type SHC/HAC enzymes or the SHC/HAC enzyme variants described herein may, for example, be from about 25° C. to about 55° C., from about 25° C. to about 50° C., from about 30° C. to about 55° C., or from about 30° C. to about 50° C.

The optimum temperature for the wild type SHC/HAC enzymes or the SHC/HAC enzyme variants describe herein may, for example, be equal to or greater than about 35° C. For example, the optimum temperature for the wild type SHC/HAC enzymes or the SHC/HAC enzyme variants may range from about 40° C. to about 50° C., for example from about 42° C. to about 48° C. or from about 44° C. to about 46° C. For example, the optimum temperature of the wild type SHC/HAC enzymes or the SHC/HAC enzyme variants may be about 45° C. The processes for making (−)-Ambrox or Ambra oxide disclosed herein may be carried out at the optimum temperature of the SHC/HAC enzyme variant.

The optimum pH range for the wild type SHC/HAC enzymes or the SHC/HAC enzyme variants may from about 5 to about 7, from about 5 to about 6.5, or from about 5 to about 6.

The optimum pH for the wild type SHC/HAC enzymes or the SHC/HAC enzyme variants may, for example, be equal to or greater than about 5.4. For example, the optimum pH for the wild type SHC/HAC enzymes or the SHC/HAC enzyme variants may range from about 5.2 to about 6.0, for example from about 5.4 to about 5.8, for example from about 5.6 to about 5.8. For example, the optimum pH of the wild type SHC/HAC enzymes or the SHC/HAC enzyme variants may be about 5.6 or about 5.8. The processes for making (−)-Ambrox or Ambra oxide disclosed herein may be carried out at the optimum pH of the wild type SHC/HAC enzymes or the SHC/HAC enzyme variant.

The optimum concentration of sodium dodecyl sulfate (SDS) in the reaction medium of the process for making (−)-Ambrox or Ambra oxide disclosed herein may, for example, be from about 0.005 w/w % to about 0.04 w/w %, from about 0.010 w/w % to about 0.10 w/w %. For example, the optimum concentration of SDS may be from about 0.040 w/w % to about 0.080 w/w %, for example about 0.050 w/w % when the substrate (e.g. EEH or BisEEH) is used at 4 g/l with cells to an OD_{650 nm} of 10. The optimum concentration of sodium dodecyl sulfate (SDS) in the reaction medium of the processes for making (−)-Ambrox or Ambra oxide disclosed herein may, for example, be from about 1.0 w/w % to about 1.5 w/w % when the substrate (e.g. EEH or BisEEH) is used at 125 g/l with 250 g/l of cells. For example, the optimum concentration of SDS may be from about 1.2 w/w % to about 1.4 w/w %, for example about 1.3 w/w % when the substrate (e.g. EEH or BisEEH) is used at 125 g/l with 250 g/l of cells.

The processes for making (−)-Ambrox or Ambra oxide disclosed herein may be carried out at the optimum temperature range or optimum temperature and/or the optimum pH range or optimum pH and/or the SDS optimum concentration range or optimum SDS concentration for the specific enzyme used, as set out in the Table 7 the Examples below.

As discussed above, it has surprisingly been found that SHC/HAC enzyme variants as defined herein (of all types 1-34) derived from an Aac SHC/HAC enzyme variant exhibit improved biological activity such as improved enzymatic activity for the conversion of EEH to (−)-Ambrox and for the conversion of BisEEH to Ambra oxide. The new SHC/HAC enzyme variants have at least one, two or three amino acid alterations in addition to the amino acid substitutions already present in the 215G2 SHC/HAC (SEQ ID NO:3) or the SHC #65 (SEQ ID NO:2) enzyme variant.

Additional SHC/HAC enzyme variants (variant of type 29) may have amino acid alterations (e.g. substitutions) at positions corresponding to positions 169, 306, 600, 81, 90, 172, 277, 431, 557 and 613 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 81, 90, 172, 277, 557, 613 of SEQ ID NO:1, 2, 3).

In particular, it is expected that SHC/HAC enzyme variants derived from other non-Aac species but having one or more of the new amino acid alterations identified at positions 169, 306, 600, 81, 90, 172, 277, 431, 557 and 613 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 81, 90, 172, 277, 557, 613 of SEQ ID NO:1, 2, 3) will also provide enzymatic activity for the conversion of EEH to (−)-Ambrox or BisEEH to Ambra oxide. In particular, it is expected that enzyme variants derived from ZmoSHC1, ZmoSHC2, BjpSHC, GmoSHC, TeISHC, ApaSHC1, BmeSHC, SaISHC, or ApaSHCA having one or more of the new amino acid alterations identified at positions 169, 306, 600, 81, 90, 172, 277, 431, 557 and 613 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 81, 90, 172, 277, 557, 613 of SEQ ID NO:1, 2, 3) will also provide enzymatic activity for the conversion of EEH to (−)-Ambrox or BisEEH to Ambra oxide.

There is therefore provided herein a process for making (−)-Ambrox by enzymatically converting EEH to (−)-Ambrox. There is also provided herein a process for making Ambra oxide by enzymatically converting E,E-bishomofarnesol to Ambra oxide. These processes may use any SHC/HAC enzyme variant described herein.

In addition, there is provided herein a SHC/HAC enzyme variant (also named variant of type 30) having at least about 70.0% identity to a wild-type SHC/HAC enzyme amino acid sequence, wherein the SHC/HAC enzyme variant amino acid sequence has one or more amino acid alterations relative to the wild-type SHC/HAC enzyme at a position selected from positions corresponding to positions 169, 306, 600, 81, 90, 172, 277, 431, 557 and 613 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 81, 90, 172, 277, 557, 613 of SEQ ID NO:1, 2, or 3).

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In particular, there is provided herein a process for preparing (−)-Ambrox or a mixture comprising (−)-Ambrox, the process comprising enzymatically converting EEH or a mixture comprising EEH to (−)-Ambrox or a mixture comprising (−)-Ambrox using a SHC/HAC variant as defined herein. All SHC/AHC variants defined herein (all types 1-34) may be used in such a process.

In particular, there is provided herein a process for preparing Ambra oxide or a mixture comprising Ambra oxide, the process comprising enzymatically converting BisEEH or a mixture comprising BisEEH to Ambra oxide or a mixture comprising Ambra oxide using a SHC/HAC variant having at least about 70.0% identity to a wild-type SHC/HAC enzyme amino acid sequence, wherein the SHC/HAC enzyme variant amino acid sequence has one or more amino acid alterations relative to the wild-type SHC/HAC enzyme at a position selected from positions corresponding to positions 169, 306, 600, 81, 90, 172, 277, 431, 557 and 613 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600, 81, 90, 172, 277, 557, 613 of SEQ ID NO:1, 2, or 3).

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In addition, there is provided herein a SHC/HAC enzyme variant (also named variant of type 31) having at least about 70.0% identity to a wild-type SHC/HAC enzyme amino acid sequence, wherein the SHC/HAC enzyme variant amino acid sequence has one or more amino acid alterations relative to the wild-type SHC/HAC enzyme at a position selected from positions corresponding to positions 169, 306, 600, 77, 92, 129, 579, 601, 605 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20. 21, 22, 23, 24, 25, 26, 27, 28, 29, 30, 31, 32, 33 and/or 34, 55-296, 307-310 corresponding to 169, 306, 600, 77, 92, 129, 579, 601, 605 of SEQ ID NO:1, 2, or 3).

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In particular, there is provided herein a process for preparing Ambra oxide or a mixture comprising Ambra oxide, the process comprising enzymatically converting BisEEH or a mixture comprising BisEEH to Ambra oxide or a mixture comprising Ambra oxide using a SHC/HAC variant having at least about 70.0% identity to a wild-type SHC/HAC enzyme amino acid sequence, wherein the SHC/HAC enzyme variant amino acid sequence has one or more amino acid alterations relative to the wild-type SHC/HAC enzyme at a position selected from positions corresponding to positions 169, 306, 600, 77, 92, 129, 579, 601, 605 of SEQ ID NO: 1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33 and/or 34, 55-296, 307-310 corresponding to 169, 306, 600, 77, 92, 129, 579, 601, 605 of SEQ ID NO:1, 2, or 3).

In this context, the identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In each of the type of SHC/HAC enzyme variants disclosed herein (types 1-34), the identity of the amino acid substituting a given position has already been earlier disclosed herein and each of these types may thus further be defined by the specific identity of the amino acid substituting the amino acid altered.

Moreover, the identity of the amino acid substituting W169, A306 and G600 of SEQ ID NO:1, 2, 3, 47, 48, 49, 50, 51, 52, 53 and/or 54 (or at a position in an amino acid sequence of a wild type SHC/HAC enzyme as SEQ ID NO: 13, 14, 15, 16, 17, 18, 19, 20, 21, 22, 23, 24, 25, 26, 27, 28, 29, 30, 31, 32, 33, 34, 55-296, 307-310 corresponding to 169, 306, 600 of SEQ ID NO:1, 2, or 3) has already been disclosed herein. These specific key positions and identity of substituting amino acids are repeated below.
the W at position 169 is replaced by G, A or V or a functional equivalent thereof, preferably wherein the W at position 169 is replaced by G,
the A at position 306 is replaced by V or a functional equivalent thereof and/or
the G at position 600 is replaced by A, V, L, I or M or a functional equivalent thereof, preferably wherein the G at position 600 is replace by M.

In addition, in an embodiment, where the amino acid residue at position 168 has a large amino acid side chain (eg Tyrosine (Q), it is replaced by an amino acid residue with a smaller side chain (eg serine).

In another embodiment, where the amino acid residue at position 168 is a serine (S) residue, it should be retained (as in WT AacSHC and WT TeISHC) Table 10: Conserved positions of amino acids located at the active site of the SHC/HAC (based on Aac SHC/HAC). Conserved positions can be seen for amino acid positions 169, 261, 312, 365, 489, 600, 609 and 612 for five different WT SHC enzymes. Differences in hydrogen binding pocket can be seen for Q, Y and C in ZmoSHC1, ZmoSHC2 and ScoSHC for the amino acid position 168.

Sequence identity to AacSHC (%) 0 45.8 38.3 44.2 51.0 active site amino acid position based on AacSHC AacSHC ZmoSHC1 ZmoSHC2 TeISHC ScoSHC

TABLE 10
Conserved positions of amino acids located at the active site
of the SHC/HAC (based on Aac SHC/HAC). Conserved positions can
be seen for amino acid positions 169, 261, 312, 365, 489, 600,
609 and 612 for five different WT SHC enzymes. Differences in
hydrogen binding pocket can be seen for Q, Y and C in ZmoSHC1,
ZmoSHC2 and ScoSHC for the amino acid position 168.

| active site | | | | | |
|---|---|---|---|---|---|
| amino acid | Sequence identity to AacSHC (%) | | | | |
| position based | 0 | 45.8 | 38.3 | 44.2 | 51.0 |
| on AacSHC | AacSHC | ZmoSHC1 | ZmoSHC2 | TelSHC | ScoSHC |
| 36 | L | V | L | L | L |
| 168 | S | Q | Y | S | C |
| 169 | W | W | W | W | W |
| 261 | I | I | I | I | I |
| 306 | A | A | P | P | A |
| 307 | S | T | C | C | C |
| 312 | W | W | W | W | W |
| 365 | F | F | F | F | F |
| 366 | Q | E | Q | E | E |
| 420 | Y | F | F | F | F |
| 489 | W | W | W | W | W |
| 600 | G | G | G | G | G |
| 607 | L | L | L | L | I |
| 609 | Y | Y | Y | Y | Y |
| 612 | Y | Y | Y | Y | Y |

conserved (bold font)
difference in hydrogen-binding pocket (italic font)

### Substrate: Homofarnesol

Homofarnesol may have isomerism as shown below.

TABLE 11
Homofarnesol isomers.

| Compound | Abbreviation | Name and Structure |
|---|---|---|
| E,E-Homofarnesol | EEH | 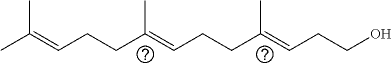 [SMILES: CC(C)=CCC/C(C)=C/CC/C(C)=C/CCO] |
| | | (3E,7E)-4,8,12-trimethyltrideca-3,7,11-trien-1-ol |
| E,Z-Homofarnesol | EZH | 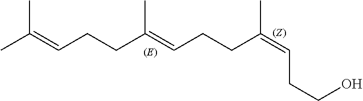 [SMILES: CC(C)=CCC/C(C)=C/CC/C(C)=C\CCO] |
| | | (3Z,7E)-4,8,12-trimethyltrideca-3,7,11-trien-1-ol |
| Z,E-Homofarnesol | ZEH | 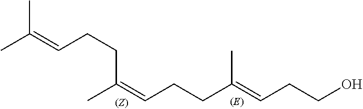 [SMILES: CC(C)=CCC/C(C)=C\CC/C(C)=C/CCO] |
| | | (3E,7Z)-4,8,12-trimethyltrideca-3,7,11-trien-1-ol |
| Z,Z-Homofarnesol | ZZH | 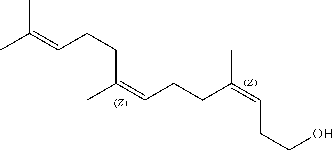 [SMILES: CC(C)=CCC/C(C)=C\CC/C(C)=C\CCO] |
| | | (3Z,7Z)-4,8,12-trimethyltrideca-3,7,11-trien-1-ol |

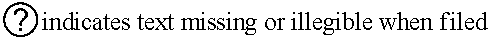

Beta-farnesene can be converted directly to E,E-homofarnesol (EEH) or indirectly to EEH via E,E-homofarnesate which is then converted to EEH. An overview on the production of (−)-Ambrox from different substrates can be found in US2012/0135477A1, WO 2010/139719, US2013.0273619A1, WO 2013/156398A1 and the Seitz PhD thesis (2012 as cited above) and Schaefer 2011 (Chemie Unserer Zeit 45, 374-388), the contents of which are incorporated herein by reference.

US2012/0135477A1 reports on the conversion of (3Z,7E) to (−)-Ambrox using ZmoSHC but according to the disclosure in Schaefer (2011) (as cited above), (7E,3Z) is only converted to 9b-epi-Ambrox (i.e. compound (III) as outlined above) and not to (−)-Ambrox. As used herein, a reference to (3Z,7E) homofarnesol is a reference to E,Z-homofarnesol which is also designated as EZH.

Whilst homofarnesol may be a mixture of four isomers, the (3Z,7Z), (3E,7Z), (3Z,7E) and (3E,7E) isomers, it seems from the literature that (−)-Ambrox is only obtained from (3E,7E) homofarnesol (see Neumann and Simon (1986) as cited above). As used herein, a reference to (3E,7E)-homofarnesol is a reference to E,E-homofarnesol which is also designated as EEH.

The starting materials for the processes described herein for preparing (−)-Ambrox may, for example, be (3E,7E)-homofarnesol or a mixture comprising (3E,7E)-homofarnesol, for example a mixture of isomers of homofarnesol comprising (3E,7E)-homofarnesol.

Preferably the homofarnesol starting material comprises a mixture of (3E,7E) and (3Z,7E), termed herein an EE:EZ isomeric mixture. An EE:EZ isomeric mixture of homofarnesol has the CAS number of 35826-67-6.

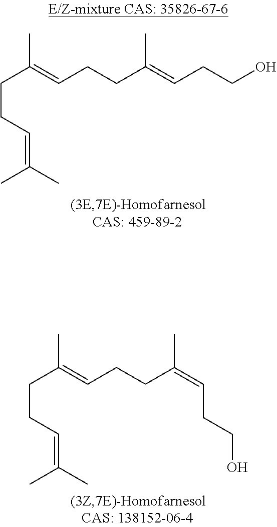 [SMILES: CC(C)=CCC/C(C)=C/CC/C(C)=C/CCO ; CC(C)=CCC/C(C)=C/CC/C(C)=C\CCO]

The homofarnesol feedstock/starting material may be a mixture of isomers. Accordingly, the homofarnesol starting material may also comprise a mixture of the four isomers EE:EZ:ZZ:ZE which corresponds with (3E,7E), (3Z,7E), (3Z,7Z) and (3E,7Z). Preferably, the homofarnesol starting material is selected from one of more of the following mixture: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)].

Preferably the homofarnesol starting material is selected from one or more of the following mixtures: [(3E,7E), (3Z,7E)] and/or [(3Z,7E), (3E/7E) and (3E,7Z)], also designated [EE:EZ] and [EE:EZ:ZE] respectively.

Accordingly, in certain embodiments, the ratio of EEH:EZH is about 100:00; 99:01; 98:02; 97:03; 96:04; 95:05; 94:06; 93:07; 92:08; 91:09; 90:10; 89:11; 88:12; 87:13; 86:14; 85:15; 84:16; 83:17; 82:18; 81:19; 80:20; 79:21; 78:22; 77:23; 76:24; 75:25; 74:26; 73:27; 72:28; 71:29; 70:30; 69:31; 68:32; 67:33; 66:34; 65:35; 64:36; 63:37; 62:38:61:39; 60:40; 59:41; 58:42; 57:43; 56:44; 55:45:54:46; 53:47:52:48; 51:49; or about 50:50. For example, the ratio of EEH:EZH may range from about 50:50 to about 100:00 or from about 50:50 to about 99:01 or from about 60:40 to about 99:1 or from about 70:30 to about 95:5 or from about 80:20 to about 95:5.

In some embodiments the homofarnesol starting material comprises >90% E,E-homofarnesol (EEH).

In other embodiments, the homofarnesol starting material comprises an EE:EZ weight ratio of 86:14.

In certain embodiments, the homofarnesol starting material comprises an EE:EZ weight ratio of 80:20.

In certain embodiments, the homofarnesol starting material comprises an EE:EZ weight ratio of 70:30.

In further embodiments, the homofarnesol starting material comprises an EE:EZ weight ratio of 69:31.

The number of homofarnesol isomers present may influence the speed of the reaction. A SHC/HAC enzyme or enzyme variant may be capable of converting E,E-homofarnesol to (−)-Ambrox from a complex mixture of homofarnesol isomers (e.g. EE:EZ:ZE:ZZ). However, a lower conversion rate may be observed, which is consistent with the view that homofarnesol isomers other than EEH may compete with EEH for access to the SHC/HAC derivative enzymes and thus may act as competitive inhibitors for the conversion of EEH to (−)-Ambrox and/or also act as alternative substrates (see for example, Eichhorn et al (2018) Adv. Synth. Catal. 360: 2339-2351, the contents of which are incorporated here by reference). Accordingly, the homofarnesol substrate may comprise a isomeric mixture of 2-4 isomers, preferably two isomers.

Accordingly, the homofarnesol substrate may consist of or consist essentially of a isomeric mixture of 2-4 isomers, preferably two isomers.

Preferably the homofarnesol substrate comprises an EE:EZ isomeric mixture. Preferably the homofarnesol substrate consists of or consists essentially of an EE:EZ isomeric mixture.

If, for example, an EE:EZ isomer mixture is used, then compounds (eg compounds II, Ill and IV as set out in Table 3) other than (−)-Ambrox are in an “oily” form (rather than a solid form) which facilates the stirring of the reaction mixture and ths resulting bioconversion process. In addition when the P1/P2/P3 SHC variants as disclosed and described herein are used to cyclise homofarnesol in a bioconversion process for producing (−)-Ambrox, a reduced cell:homofarnesol substrate ratio may be used leading to a reduction in the amount of solid material (eg cells and (−)-Ambrox)) in the reaction mixture which is advantageous not only for the stirrability of the reaction mixture but also for downstream processing (especially as regards filtration) as the solid cell mass is reduced meaning that there is less likelihood of filtration membrane congestion and the like.

In an embodiment, a cell to EEH ratio of 0.5 or 0.4 or 0.3 or 0.2 or 0.1 is used. In an embodiment, when such a cell to EEH ratio is used, the EEH conversion is of at least 50%, 60%, 70%, 80%, 90%, 95% or 98%. In an embodiment, such a cell to EEH ratio is used even when 100 or 150 or 200 or even 250 g/l EEH is present. In an embodiment when such cell to EEH ratio is used and such concentration of EEH is used a conversion of up to 4.5 or 5.0 or 5.5 or even 5.9 or 6.0 g of EEH is converted per gram of cells. Such results have been obtained in the experimental part (see example 9). In the context of the reference to the cell to EEH ratio, EEH may be present in a mixture with other isomer of homofarnesol as described herein. We refer to the total concentration of EEH present even if other isomer of homofarnesol may be present.

Bishomofarnesol Bishomofarnesol may have isomerism as shown in Table 12 below.

TABLE 12
Bishomofarnesol isomers.

| Compound | Abbreviation | Name and Structure |
|---|---|---|
| E,E-Bisomofarnesol | BisEEH | 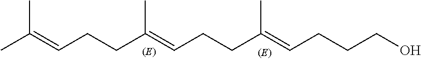 [SMILES: CC(C)=CCC/C(C)=C/CC/C(C)=C/CCCO] |
| | | (4E,8E)-5,9,13-trimethyltetradeca-4,8,12-trien-1-ol |
| E,Z-Bishomofarnesol | BisEZH | 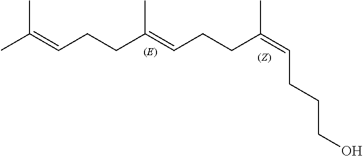 [SMILES: CC(C)=CCC/C(C)=C/CC/C(C)=C\CCCO] |
| | | (4Z,8E)-5,9,13-trimethyltetradeca-4,8,12-trien-1-ol |
| Z,E-Bishomofarnesol | BisZEH | 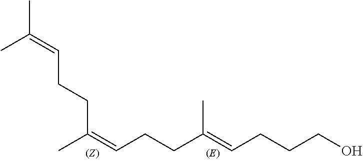 [SMILES: CC(C)=CCC/C(C)=C\CC/C(C)=C/CCCO] |
| | | (4E,8Z)-5,9,13-trimethyltetradeca-4,8,12-trien-1-ol |
| Z,Z-Bishomofarnesol | BisZZH | 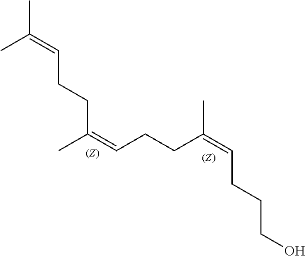 [SMILES: CC(C)=CCC/C(C)=C\CC/C(C)=C\CCCO] |
| | | (4Z,8Z)-5,9,13-trimethyltetradeca-4,8,12-trien-1-ol |

Bishomofarnesol may be produced from E-Nerolidol as described in WO 2021/110848. For example, bishomofarnesol may be produced as a mixture of two or more isomers (e.g. a mixture of E,E-bishomofarnesol and E,Z-bishomofarnesol). Whilst bishomofarnesol may present as a mixture of four isomers (the (Z,Z), (E,Z), (Z,E) and (E,E) isomers) it seems that Ambra oxide is only obtained from E,E-bishomofarnesol.

The starting materials for the processes described herein for preparing Ambra oxide may, for example, be E,E-bishomofarnesol or a mixture comprising E,E-bishomofarnesol, for example a mixture of isomers of bishomofarnesol comprising E,E-bishomofarnesol.

Preferably the bishomofarnesol starting material comprises a mixture of (BisEEH) and (BisEZH), termed herein an EE:EZ isomeric mixture.

The bishomofarnesol feedstock/starting material may be a mixture of isomers. Accordingly, the bishomofarnesol starting material may also comprise a mixture of the four isomers EE:EZ:ZZ:ZE.

Accordingly, in certain embodiments, the ratio of BisEEH:BisEZH is about 100:00; 99:01; 98:02; 97:03; 96:04; 95:05; 94:06; 93:07; 92:08; 91:09; 90:10; 89:11; 88:12; 87:13; 86:14; 85:15; 84:16; 83:17; 82:18; 81:19; 80:20; 79:21; 78:22; 77:23; 76:24; 75:25; 74:26; 73:27; 72:28; 71:29; 70:30; 69:31; 68:32; 67:33; 66:34; 65:35; 64:36; 63:37; 62:38:61:39; 60:40; 59:41; 58:42; 57:43; 56:44; 55:45:54:46; 53:47:52:48; 51:49; or about 50:50. For example, the ratio of BisEEH:BisEZH may range from about 50:50 to about 100:00 or from about 50:50 to about 99:01 or from about 60:40 to about 99:1 or from about 70:30 to about 95:5 or from about 80:20 to about 95:5.

In some embodiments preferably the bishomofarnesol starting material comprises >90% E,E-bishomofarnesol (EEH).

In other embodiments, the bishomofarnesol starting material comprises an BisEEH:BisEZH weight ratio of 86:14. In certain embodiments, the bishomofarnesol starting material comprises a BisEEH:BisEZH weight ratio of 80:20.

In certain embodiments, the bishomofarnesol starting material comprises a BisEEH:BisEZH weight ratio of 70:30.

In further embodiments, the bishomofarnesol starting material comprises an BisEEH:BisEZH weight ratio of 69:31 or 65:35 The number of bishomofarnesol isomers present may influence the speed of the reaction. A SHC/HAC enzyme or enzyme variant may be capable of converting E,E-bishomofarnesol to Ambra oxide from a complex mixture of bishomofarnesol isomers (e.g. EE:EZ:ZE:ZZ). However, a lower conversion rate may be observed, which is consistent with the view that bishomofarnesol isomers other than BisEEH may compete with BisEEH for access to the SHC/HAC enzyme or enzyme variant and thus may act as competitive inhibitors for the conversion of BisEEH to Ambra oxide and/or also act as alternative substrates. Accordingly, the bishomofarnesol substrate may comprise a isomeric mixture of 2-4 isomers, preferably two isomers.

Accordingly, the bishomofarnesol substrate may consist of or consist essentially of a isomeric mixture of 2-4 isomers, preferably two isomers.

Preferably the bishomofarnesol substrate comprises an EE:EZ isomeric mixture.

Preferably the bishomofarnesol substrate consists of or consists essentially of an EE:EZ isomeric mixture.

### Nucleic Acids and Methods of Making Nucleic Acids

There is further provided herein nucleic acids encoding a SHC/HAC enzyme variant as described herein. (of types 1 to 30). The nucleic acid may, for example, be an isolated nucleic acid.

In particular, there is provided herein a construct comprising a nucleic acid sequence encoding a SHC/HAC enzyme variant as described herein. As used herein, a “construct” is an artificially created segment of nucleic acid that is to be transfected into a target cell. The construct may comprise the nucleic acid encoding the SHC/HAC enzyme or enzyme variant and an expression controller (e.g. promoter).

There is further provided herein a vector comprising a construct as described herein. As used herein, a “vector” is a DNA molecule that is used as a vehicle to artificially carry foreign genetic material into a cell where it can be replicated and/or expressed. The vector may, for example, be a plasmid, a viral vector, a cosmid, or an artificial chromosome.

The terms “construct” and “vector” may overlap, for example where the construct is a plasmid.

In particular, there is provided herein a nucleic acid encoding an amino acid sequence of any one of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and as identified below: In an embodiment, SEQ ID NO: 38 is the nucleotide sequence encoding the AacSHC P1 represented by SEQ ID NO:4.

In an embodiment, SEQ ID NO: 39 is the nucleotide sequence encoding the AacSHC P2 represented by SEQ ID NO:5.

In an embodiment, SEQ ID NO: 40 is the nucleotide sequence encoding the AacSHC P3 represented by SEQ ID NO:6.

In an embodiment, SEQ ID NO: 41 is the nucleotide sequence encoding the SHC #65 P1 variant represented by SEQ ID NO:7.

In an embodiment, SEQ ID NO: 42 is the nucleotide sequence encoding the SHC #65 P2 variant represented by SEQ ID NO:8.

In an embodiment, SEQ ID NO: 43 is the nucleotide sequence encoding the SHC #65 P3 variant represented by SEQ ID NO:9.

In an embodiment, SEQ ID NO: 44 is the nucleotide sequence encoding the 215G2 P1 variant represented by SEQ ID NO:10.

In an embodiment, SEQ ID NO: 45 is the nucleotide sequence encoding the 215G2 P2 variant represented by SEQ ID NO:11.

In an embodiment, SEQ ID NO: 46 is the nucleotide sequence encoding the 215G2 P3 variant represented by SEQ ID NO:12.

The term “nucleic acid” or “nucleic acid molecule” as used herein shall specifically refer to polynucleotides of the disclosure which can be DNA, cDNA, genomic DNA, synthetic DNA, or RNA, and can be double-stranded or single-stranded, the sense and/or an antisense strand. The term “nucleic acid” or “nucleic acid molecule” shall particularly apply to the polynucleotide(s) as used herein, e.g. as full-length nucleotide sequence or fragments or parts thereof, which encode a polypeptide with enzymatic activity, e.g. an enzyme of a metabolic pathway, or fragments or parts thereof, respectively.

The term also includes a separate molecule such as a cDNA where the corresponding genomic DNA has introns and therefore a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein (e.g. a His tag), mutein, or fragment of a given protein; and a nucleic acid which is a degenerate variant of a cDNA or a naturally occurring nucleic acid. In addition, it includes a recombinant nucleotide sequence that is part of a hybrid gene, i.e. a gene encoding a non-naturally occurring fusion protein. Fusion proteins can add one or more amino acids (such as but not limited to Histidine (His)) to a protein, usually at the N-terminus of the protein but also at the C-terminus or fused within regions of the protein. Such fusion proteins or fusion vectors encoding such proteins typically serve three purposes: (i) to increase production of recombinant proteins; (ii) to increase the solubility of the recombinant protein; and (iii) to aid in the purification of the recombinant protein by providing a ligand for affinity purification.

The term “nucleic acid” or “nucleic acid molecule” also includes codon optimised sequences suitable for expression in a particular microbial host cell (e.g. E. coli host cell). As used herein, the term “codon optimized” means a nucleic acid protein coding sequence which has been adapted for expression in a prokaryotic or a eukaryotic host cell, particularly bacterial host cells such as E. coli host cells by substitution of one or more or preferably a significant number of codons with codons that are more frequently used in bacterial (e.g. E. coli) host cell genes.

In this regard, the nucleotide sequence encoding the reference amino acid sequence (SEQ ID NO: 1 or 2 or 3 or the gene can be codon-optimized for the selected host organisms, such as e.g. E. coli.

A nucleotide sequence encoding the wild type Aac SHC (SEQ ID NO:1) is SEQ ID NO:35.

A nucleotide sequence encoding the parent SHC (SEQ ID NO:2) is SEQ ID NO:36.

A nucleotide sequence encoding the other patent SHC (SEQ ID NO:3) is SEQ ID NO:37.

A ribonucleic acid (RNA) molecule can be produced by in vitro transcription. Segments of DNA molecules are also considered within the scope of the disclosure, and can be produced by, for example, the polymerase chain reaction (PCR) or generated by treatment with one or more restriction endonucleases. Segments of a nucleic acid molecule may be referred to as DNA fragments of a gene, in particular those that are partial genes. A fragment can also contain several open reading frames (ORF), either repeats of the same ORF or different ORF's. The term shall specifically refer to coding nucleotide sequences, but shall also include nucleotide sequences which are non-coding, e.g. untranscribed or untranslated sequences, or encoding polypeptides, in whole or in part. The genes as used herein, e.g. for assembly, diversification or recombination can be non-coding sequences or sequences encoding polypeptides or protein encoding sequences or parts or fragments thereof having sufficient sequence length for successful recombination events. More specifically, said genes have a minimum length of 3 bp, preferably at least 100 bp, more preferred at least 300 bp. It will be apparent from the foregoing that a reference to an isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice. An isolated nucleic acid molecule of the present disclosure encompasses segments that are not found as such in the natural state.

As used herein, the term “isolated DNA” can refer to (1) a DNA that contains sequence not identical to that of any naturally occurring sequence, a polynucleotide or nucleic acid which is not naturally occurring, (e.g. is made by the artificial combination (e.g. artificial manipulation of isolated segments of nucleic acids, e.g. by genetic engineering techniques) of two otherwise separated segments of sequences through human intervention) or (2), in the context of a DNA with a naturally-occurring sequence (e.g. a cDNA or genomic DNA), a DNA free of at least one of the genes that flank the gene containing the DNA of interest in the genome of the organism in which the gene containing the DNA of interest naturally occurs.

The term “isolated DNA” as used herein, specifically with respect to nucleic acid sequences may also refer to nucleic acids or polynucleotides produced by recombinant DNA techniques, e.g. a DNA construct comprising a polynucleotide heterologous to a host cell, which is optionally incorporated into the host cell. A chimeric nucleotide sequence may specifically be produced as a recombinant molecule. The term “recombination” shall specifically apply to assembly of polynucleotides, joining together such polynucleotides or parts thereof, with or without recombination to achieve a cross-over or a gene mosaic. For example, it is performed to join together nucleic acid segments of desired functions to generate a desired combination of functions. A recombinant gene encoding a polypeptide described herein may include the coding sequence for that polypeptide, operably linked, in sense orientation, to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence.

The term “recombinant” as used herein, specifically with respect to enzymes shall refer to enzymes produced by recombinant DNA techniques, i.e. produced from cells transformed by an exogenous DNA construct encoding the desired enzyme. “Synthetic” enzymes are those prepared by chemical synthesis. A chimeric enzyme may specifically be produced as recombinant molecule. The term “recombinant DNA” therefore includes a recombinant DNA incorporated into a vector into an autonomously replicating plasmid or virus, or into the genomic DNA of a prokaryote or eukaryote (or the genome of a homologous cell, at a position other than the natural chromosomal location).

In a further aspect the nucleic acid molecule(s) of the present disclosure is/are operatively linked to expression control sequences allowing expression in prokaryotic and/or eukaryotic host cells. As used herein, “operatively linked” means incorporated into a genetic construct so that expression control sequences effectively control expression of a coding sequence of interest. The transcriptional/translational regulatory elements referred to above include but are not limited to inducible and non-inducible, constitutive, cell cycle regulated, metabolically regulated promoters, enhancers, operators, silencers, repressors and other elements that are known to those skilled in the art and that drive or otherwise regulate gene expression. Such regulatory elements include but are not limited to regulatory elements directing constitutive expression or which allow inducible expression like, for example, CUP-1 promoter, the tet-repressor as employed, for example, in the tet-on or tet-off systems, the lac system, the trp system regulatory elements. By way of example, Isopropyl p-D-1-thiogalactopyranoside (IPTG) is an effective inducer of gene expression in the concentration range of 100 μM to 1.0 mM. This compound is a molecular mimic of allolactose, a lactose metabolite that triggers transcription of the lac operon, and it is therefore used to induce gene expression when the gene is under the control of the lac operator. Another example of a regulatory element which induces gene expression is lactose. Similarly, the nucleic acid molecule(s) of the present disclosure can form part of a hybrid gene encoding additional polypeptide sequences, for example, a sequence that functions as a marker or reporter. Examples of marker and reporter genes including beta-lactamase, chloramphenicol acetyltransferase (CAT), adenosine deaminase (ADA), aminoglycoside phosphotransferase dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). As with many of the standard procedures associated with the practice of the disclosure, skilled artisans will be aware of additional useful reagents, for example, additional sequences that can serve the function of a marker or reporter.

In some embodiments, the present disclosure provides a recombinant polynucleotide encoding the SHC/HAC enzyme or variant thereof, which may be inserted into a vector for expression and optional purification. One type of vector is a plasmid representing a circular double stranded DNA loop into which additional DNA segments are ligated. Certain vectors can control the expression of genes to which they are functionally linked. These vectors are called “expression vectors”. Usually expression vectors suitable for DNA recombination techniques are of the plasmid type. Typically, an expression vector comprises a gene such as the SHC/HAC enzyme or variant thereof as described herein. In the present description, the terms “plasmid” and “vector” may be used interchangeably since the plasmid is the vector type most often used. Such vectors can include DNA sequences which include but are not limited to DNA sequences that are not naturally present in the host cell, DNA sequences that are not normally transcribed into RNA or translated into a protein (“expressed”) and other genes or DNA sequences which one desires to introduce into the non-recombinant host. It will be appreciated that typically the genome of a recombinant host described herein is augmented through the stable introduction of one or more recombinant genes. However, autonomous or replicative plasmids or vectors can also be used within the scope of this disclosure. Moreover, the present disclosure can be practiced using a low copy number, e.g. a single copy, or high copy number (as exemplified herein) plasmid or vector.

In a preferred embodiment, the vector of the present disclosure includes plasmids, phagemids, phages, cosmids, artificial bacterial and artificial yeast chromosomes, knock-out or knock-in constructs, synthetic nucleic acid sequences or cassettes and subsets may be produced in the form of linear polynucleotides, plasmids, megaplasmids, synthetic or artificial chromosomes, such as plant, bacterial, mammalian or yeast artificial chromosomes.

It is preferred that the proteins encoded by the introduced polynucleotide are expressed within the cell upon introduction of the vector. The diverse gene substrates may be incorporated into plasmids. The plasmids are often standard cloning vectors, e.g. bacterial multicopy plasmids. The substrates can be incorporated into the same or different plasmids. Often at least two different types of plasmid having different types of selectable markers are used to allow selection for cells containing at least two types of vectors.

Typically, bacterial or yeast cells may be transformed with any one or more nucleotide sequences as is well known in the art. For in vivo recombination, the gene to be recombined with the genome or other genes is used to transform the host using standard transforming techniques. In a suitable embodiment DNA providing an origin of replication is included in the construct. The origin of replication may be suitably selected by the skilled person. Depending on the nature of the genes, a supplemental origin of replication may not be required if sequences are already present with the genes or genome that are operable as origins of replication themselves.

### Host Cells, Methods of Making Host Cells, and Methods of Making Ambrox and Ambra Oxide Using Host Cells

There is further provided herein a recombinant host cell comprising a nucleic acid sequence or a construct or a vector as described herein. There is further provided herein a recombinant host cell that produces a SHC/HAC enzyme or enzyme variant as described herein. A recombinant host cell may also be named an engineered cell or a cell.

The processes described herein for producing (−)-Ambrox or Ambra oxide may, for example, comprise culturing a recombinant host cell as described herein. As used herein, the term “culturing” refers to a process of producing living cells such that they produce a SHC/HAC enzyme or enzyme variant as described herein (such as those of the types 1 to 30) that can be used in a process for producing (−)-Ambrox or Ambra oxide as described herein. It is not necessary for the cells to divide and replicate themselves, although this is not excluded.

A bacterial or yeast cell may be transformed by exogenous or heterologous DNA when such DNA has been introduced inside the cell. The transforming DNA may or may not be integrated, i.e. covalently linked into the genome of the cell. In prokaryotes, and yeast, for example, the transforming DNA may be maintained on an episomal element such as a plasmid. With respect to eukaryotic cells, a stably transfected cell is one in which the transfected DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

Generally, the introduced DNA is not originally resident in the host that is the recipient of the DNA, but it is within the scope of the disclosure to isolate a DNA segment from a given host, and to subsequently introduce one or more additional copies of that DNA into the same host, e.g. to enhance production of the product of a gene or alter the expression pattern of a gene. In some instances, the introduced DNA will modify or even replace an endogenous gene or DNA sequence, e.g. by homologous recombination or site-directed mutagenesis. Suitable recombinant hosts include microorganisms, plant cells, and plants.

The present disclosure also features recombinant hosts. The term “recombinant host”, also referred to as a “genetically modified host cell” or a “transgenic cell” denotes a host cell that comprises a heterologous nucleic acid or the genome of which has been augmented by at least one incorporated DNA sequence. A host cell of the present disclosure may be genetically engineered with the polynucleotide or the vector as outlined above.

The host cells that may be used for purposes of the disclosure include but are not limited to prokaryotic cells such as bacteria (for example, E. coli and B. subtilis), which may, for example, be transformed with, for example, recombinant bacteriophage DNA, plasmid DNA, bacterial artificial chromosome, or cosmid DNA expression vectors containing the polynucleotide molecules of the disclosure; simple eukaryotic cells like yeast (for example, Saccharomyces and Pichia), which may, for example, be transformed with, for example, recombinant yeast expression vectors containing the polynucleotide molecule of the disclosure. Depending on the host cell and the respective vector used to introduce the polynucleotide of the disclosure the polynucleotide can integrate, for example, into the chromosome or the mitochondrial DNA or can be maintained extrachromosomally like, for example, episomally or can be only transiently comprised in the cells.

The term “cell” as used herein in particular with reference to genetic engineering and introducing one or more genes or an assembled cluster of genes into a cell, or a production cell is understood to refer to any prokaryotic or eukaryotic cell. Prokaryotic and eukaryotic host cells are both contemplated for use according to the disclosure, including bacterial host cells like E. coli or Bacillus sp, yeast host cells, such as S. cerevisiae, insect host cells, such as Spodoptora frugiperda or human host cells, such as HeLa and Jurkat.

Specifically, the cell is a eukaryotic cell, preferably a fungal, mammalian or plant cell, or a prokaryotic cell. Suitable eukaryotic cells include, for example, without limitation, mammalian cells, yeast cells, or insect cells (including Sf9), amphibian cells (including melanophore cells), or worm cells including cells of Caenorhabditis (including Caenorhabditis elegans). Suitable mammalian cells include, for example, without limitation, COS cells (including Cos-1 and Cos-7), CHO cells, HEK293 cells, HEK293T cells, HEK293 T-Rex™ cells, or other transfectable eucaryotic cell lines. Suitable bacterial cells include without limitation E. coli.

Preferably prokaryotes, such as E. coli, Bacillus, Streptomyces, or mammalian cells, like HeLa cells or Jurkat cells, or plant cells, like Arabidopsis, may be used.

The cell may, for example, be selected from prokaryotic, yeast, plant, and/or insect host cells. Preferably the cell is an Aspergillus sp. or a fungal cell, preferably, it can be selected from the group consisting of the genera Saccharomyces, Candida, Kluyveromyces, Hansenula, Schizosaccharomyces, Yarrowia, Pichia and Aspergillus.

Preferably, the cell us a bacteria cells, for example, having a genus selected from Escherichia, Streptomyces, Bacillus, Pseudomonas, Lactobacillus and Lactococcus. For example, the bacteria may be E. coli.

Preferably the E. coli host cell is an E. coli host cell which is recognized by the industry and regulatory authorities (including but not limited to an E. coli K12 host cell or an E. coli BL21 host cell).

One preferred host cell to use with the present disclosure is E. coli, which may be recombinantly prepared as described herein. Thus, the recombinant host may be a recombinant E. coli host cell. There are libraries of mutants, plasmids, detailed computer models of metabolism and other information available for E. coli, allowing for rational design of various modules to enhance product yield. Methods similar to those described above for Saccharomyces can be used to make recombinant E. coli micro-organisms.

In one embodiment, the recombinant E. coli microorganism comprises nucleotide sequences encoding SHC/HAC enzyme or enzyme variant genes.

Preferably, the recombinant E. coli microorganism comprises a vector construct as described herein. In another preferred embodiment, the recombinant E. coli microorganism comprises nucleotide sequences encoding the SHC/HAC enzymes and enzyme variants disclosed herein.

Another preferred host cell to use with the present disclosure is S. cerevisiae which is a widely used chassis organism in synthetic biology. Thus, the recombinant host may be S. cerevisiae. There are libraries of mutants, plasmids, detailed computer models of metabolism and other information available for S. cerevisiae, allowing for rational design of various modules to enhance product yield. Methods are known for making recombinant S. cerevisiae microorganisms.

Culturing of cells may be performed in a conventional manner. The culture medium may contain a carbon source, at least one nitrogen source and inorganic salts, and vitamins are added to it. The constituents of this medium can be the ones which are conventionally used for culturing the species of microorganism in question. Carbon sources of use in the instant method include any molecule that can be metabolized by the recombinant host cell to facilitate growth and/or production of (−)-Ambrox or Ambra oxide. Examples of suitable carbon sources include, but are not limited to, sucrose (e.g. as found in molasses), fructose, xylose, glycerol, glucose, cellulose, starch, cellobiose or other glucose containing polymer.

In embodiments employing yeast as a host, for example, carbon sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g. protein, and then provided with a source of carbon only during the fed-batch phase.

The suitability of a recombinant host cell microorganism for use in the methods of the present disclosure may be determined by simple test procedures using well known methods. For example, the microorganism to be tested may be propagated in a rich medium (e.g. LB-medium, Bacto-tryptone yeast extract medium, nutrient medium and the like) at a pH, temperature and under reaction conditions commonly used for propagation of the microorganism. Once recombinant microorganisms (i.e. recombinant host cells) are selected that produce the desired products of bioconversion, the products are typically produced by a production host cell line on the large scale by suitable expression systems and fermentations, e.g. by microbial production in cell culture. In one embodiment of the present disclosure, a defined minimal medium such as M9A is used for cell cultivation.

The components of M9A medium comprise: 14 g/l KH₂PO₄, 16 g/l K2HPO₄, 1 g/l Na₃Citrate·2H₂O, 7.5 g/l (NH₄)₂SO₄, 0.25 g/l MgSO₄·7H₂O, 0.015 g/l CaCl₂·2H₂O, 5 g/l glucose and 1.25 g/l yeast extract).

In another embodiment of the present disclosure, nutrient rich medium such as LB was used. The components of LB medium comprise: 10 g/l tryptone, 5 g/l yeast extract, 5 g/l NaCl. Other examples of Mineral Medium and M9 Mineral Medium are disclosed, for example, in U.S. Pat. No. 6,524,831B2 and US 2003/0092143A1.

Another example of a minimal medium may be prepared as follows: for 350 ml culture: to 35 ml citric acid/phosphate stock (133 g/l KH₂PO₄, 40 g/l (NH₄)₂HPO₄, 17 g/l citric acid·H₂O with pH adjusted to 6.3) was added 307 ml H₂O, the pH adjusted to 6.8 with 32% NaOH as required. After autoclaving 0.850 ml 50% MgSO₄, 0.035 ml trace elements solution (see below) solution, 0.035 ml Thiamin solution and 7 ml 20% glucose were added.

Trace elements solution: 50 g/l Na₂EDTA·2H₂O, 20 g/l FeSO₄·7H₂O, 3 g/l H₃BO₃, 0.9 g/l MnSO₄·2H₂O, 1.1 g/l COCl₂, 80 g/L CuCl₂, 240 g/l NiSO₄·7H₂O, 100 g/l KI, 1.4 g/l (NH₄)₆Mo₇O₂₄·4H₂O, 1 g/l ZnSO₄·7H₂O, in deionized water

Thiamin solution: 2.25 g/l Thiamin·HCl in deionized water

MgSO₄ solution: 50% (w/v) MgSO₄·7H₂O in deionized water

The recombinant microorganism may be grown in a batch, fed batch or continuous process or combinations thereof. Typically, the recombinant microorganism is grown in a fermentor at a defined temperature(s) in the presence of a suitable nutrient source, e.g. a carbon source, for a desired period of time to produce sufficient enzyme to convert homofarnesol to Ambrox or to convert bishomofarnesol to Ambra oxide and to produce a desired amount of Ambrox including (−)-Ambrox or a desired amount of Ambra oxide of formula (X). The recombinant host cells may be cultivated in any suitable manner, for example by batch cultivation or fed-batch cultivation.

As used herein, the term “batch cultivation” is a cultivation method in which culture medium and/or nutrients is/are neither added nor withdrawn during the cultivation.

As used herein, the term “fed-batch” means a cultivation method in which culture medium and/or nutrients is/are added during the cultivation but no culture medium is withdrawn.

One embodiment of the present disclosure provides a method (or a process) of producing (−)-Ambrox or Ambra oxide in a cellular system comprising producing SHC/HAC enzymes or enzyme variants under suitable conditions in a cellular system, adding homofarnesol or bishomofarnesol to the cellular system, converting homofarnesol to (−)-Ambrox or converting bishomofarnesol to Ambra oxide using the SHC/HAC enzymes or enzyme variants produced using the cellular system, collecting (−)-Ambrox or Ambra oxide from cellular system and optionally isolating the (−)-Ambrox materials or Ambra oxide of formula (X) from the system.

Each of the SHC/HAC enzyme variants earlier disclosed herein may be used in this process, (especially each of the types 1-30 disclosed earlier herein) of SHC/HAC enzyme variants.

In an embodiment, there is provided a process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using an SHC/HAC enzyme variant amino acid sequence that has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and/or G600 of SEQ ID NO: 1, 2, 3 or at a position in an amino acid sequence of a wild type SHC/HAC enzyme corresponding to W169, A306 and/or G600 of SEQ ID NO:1, 2, or 3.

In an embodiment, there is provided a process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant having an amino acid sequence with at least 30%, identity or similarity to SEQ ID NO: 1, SEQ ID NO:2, SEQ ID NO:3, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and/or SEQ ID NO: 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 and having amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 at positions corresponding to positions W169, A306 and/or G600 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 or at a position in an amino acid sequence of a wild type SHC/HAC enzyme as SEQ ID NO: 13, SEQ ID NO: 14, SEQ ID NO: 15, SEQ ID NO: 16, SEQ ID NO: 17, SEQ ID NO: 18, SEQ ID NO: 19 or SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO:55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 corresponding to W169, A306 and/or G600 of SEQ ID NO:1, 2, or 3.

In this context, the identity or similarity may be at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the amino acid alterations in the SHC/HAC variants used in said process are the following:
the W at position 169 of SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, A or V or a functional equivalent thereof, preferably wherein the W at position 169 is replaced by G,
the A at position 306 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by A, V, L, I or M or a functional equivalent thereof.

In an embodiment, the amino acid alterations in the SHC/HAC variants used in said process are the following:
the amino acid at position 168 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is S or the amino acid at a position in an amino acid sequence of a wild type SHC corresponding to 168 of SEQ ID NO:1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is S.

In an embodiment, the SHC/HAC variants used in said process are the following:
the SHC/HAC enzyme variant has an amino acid sequence having at least 70.0% identity to SEQ ID NO: 1, 4, 5, 6 or 350 and has at least one of the following mutations: W169G, A306V and G600M or
the SHC/HAC enzyme variant has an amino acid sequence having at least 70.0% identity to SEQ ID NO: 1, 4, 5, 6 or 350 and have mutation W169G or
the SHC/HAC enzyme variant has an amino acid sequence having at least 70.0% identity to SEQ ID NO: 1, 4, 5, 6 or 350 and have the following mutations: W169G and G600M or
the SHC/HAC enzyme variant has an amino acid sequence having at least 70.0% identity to SEQ ID NO: 1, 4, 5, 6 or 350 and has at least one of the following mutations: W169G and A306V or
the SHC/HAC enzyme variant has an amino acid sequence having at least 70.0% identity to SEQ ID NO: 1, 4, 5, 6 or 350 and have the following mutations: W169G, A306V and G600M or
the SHC/HAC enzyme variant has an amino acid sequence having at least 70.0% identity to SEQ ID NO: 7, 8, 9 or 386, has the following mutations: M132R, A224V, 1432T, A557T, R613S, and has at least one of the following mutations: W169G, A306V and G600M or
the SHC/HAC enzyme variant has an amino acid sequence having at least 70.0% identity to SEQ ID NO: 10, 11, 12 or 385 has the following mutations: M132R, A224V, 1432T and has at least one of the following mutations: W169G, A306V and G600M.

In this context, the identity or similarity may be at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the amino acid alterations in the SHC/HAC variants used in said process have an amino acid sequence comprising or consisting of or essentially consisting of SEQ ID NO: 4, 5, 6, 7, 8, 9, 10, 11, 12, 305, 306, 302, 304, 311, 312, 313, 353, 354, 355, 356, 357, 358, 359, 360, 350, 386 or 385.

In an embodiment, the amino acid alterations in the SHC/HAC variants used in said process have an amino acid sequence comprising or consisting of or essentially consisting of SEQ ID NO: 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 350, 351 or 352.

Expression of other nucleotide sequences may serve to enhance the method. The bioconversion method can include the additional expression of other nucleotide sequences in the cellular system. The expression of other nucleotide sequences may enhance the bioconversion pathway for making (−)-Ambrox or Ambra oxide. A further embodiment of the present disclosure is a bioconversion method of making (−)-Ambrox or Ambra oxide comprising growing host cells comprising SHC/HAC enzyme or enzyme variant genes, producing SHC/HAC enzyme variants in the host cells, feeding homofarnesol (e.g. EEH) or bishomofarnesol (e.g. bisEEH) to the host cells, incubating the host cells under conditions of pH, temperature and solubilizing agent suitable to promote the conversion of homofarnesol to (−)-Ambrox or conversion of bishomofarnesol to Ambra oxide and collecting (−)-Ambrox or Ambra oxide of formula (X). The production of the SHC/HAC enzymes or enzyme variants in the host cells provides a method of making (−)-Ambrox or Ambra oxide of formula (X) when homofarnesol or bishomofarnesol is added to the host cells under suitable reaction conditions. Achieved conversion may be enhanced by adding more biocatalyst and SDS to the reaction mixture.

The recombinant host cell microorganism may be cultured in a number of ways in order to provide cells in suitable amounts producing the SHC/HAC enzymes or enzyme variants for the subsequent bioconversion step. Since the microorganisms applicable for the bioconversion step vary broadly (e.g. yeasts, bacteria and fungi), culturing conditions are, of course, adjusted to the specific requirements of each species and these conditions are well known and documented. Any of the art known methods for growing cells of recombinant host cell microorganisms may be used to produce the cells utilizable in the subsequent bioconversion step of the present disclosure.

Typically, the cells are grown to a particular density (measurable as optical density (OD)) to produce a sufficient biomass for the bioconversion reaction.

The cultivation conditions chosen influence not only the amount of cells obtained (the biomass) but the quality of the cultivation conditions also influences how the biomass becomes a biocatalyst. The recombinant host cell microorganism expressing the SHC/HAC enzyme or enzyme variant gene and producing the SHC/HAC enzyme or enzyme variant is termed a biocatalyst which is suitable for use in a bioconversion reaction. In some embodiments the biocatalyst is a recombinant whole cell producing SHC/HAC enzymes or enzyme variants or it may be in suspension or an immobilized format. In other embodiments, the biocatalyst is a membrane fraction or a liquid fraction prepared from the recombinant whole cell producing the SHC/HAC enzyme or enzyme variant (as disclosed for example in Seitz et al 2012—as cited above). The recombinant biocatalyst producing SHC/HAC enzymes or enzyme variants includes whole cells collected from the fermenter (for the bioconversion reaction) or the cells in the fermenter (which are then used in a one-pot reaction). The recombinant biocatalyst producing SHC/HAC enzymes or enzyme variants can include intact recombinant whole cell and/or cell debris and/or permeabilized cells and/or spheroplasts (see https://chemrxiv.org/engage/chemrxiv/search-dashboard?text=Hauer) and/or cell extracts and/or cell membranes. Either way, the SHC/HAC enzyme or enzyme variant is associated with a membrane (such as a cell membrane) in some way in order to receive and/or interact with a substrate (e.g. homofarnesol or bishomofarnesol), which membrane (such as a cell membrane) can be part of or comprise a whole cell (e.g. a recombinant whole cell). The SHC/HAC enzymes or enzyme variants may also be in an immobilized form (e.g. associated with an enzyme carrier) which allows the SHC/HAC enzymes or enzyme variants to interact with a substrate (e.g. homofarnesol or bishomofarnesol). The SHC/HAC enzymes or enzyme variants may also be used in a soluble form.

In a preferred embodiment, the process of the disclosure is carried out using recombinant whole cells as biocatalyst. Without wishing to be bound by theory the use of whole cells in the appropriate amount is advantageous because (i) the integrity of the SHC enzyme (which is membrane associated) appears to be maintained, and (ii) the cells may have a positive contribution in the distribution of an oily (ie liquid) substrate in the reaction. The use of a recombinant whole cell as biocatalyst may assist in the distribution of the substrate (which is in liquid form) but not the (−)-Ambrox product (which is in a solid form).

In one embodiment, the biocatalyst is produced in sufficient amounts (to create a sufficient biomass), harvested and washed (and optionally stored (e.g. refrigerated, frozen or lyophilized)) before starting the bioconversion step.

In a further embodiment, the cells are produced in sufficient amounts (to create a sufficient biocatalyst) and the reaction conditions are then adjusted without the need to harvest and wash the biocatalyst for the bioconversion reaction. This one step (or “one pot”) method is advantageous as it simplifies the process while possibly reducing costs. The culture medium used to grow the cells is also suitable for use in the bioconversion reaction provided that the reaction conditions are adjusted to facilitate the bioconversion reaction.

The optimum pH for growing the cells is in the range of 6.0-7.0. The optimum pH for the bioconversion reaction is dependent on the type of SHC/HAC enzyme or enzyme variant used in the bioconversion reaction. The pH is regulated using techniques which are well known to the Skilled Person.

Whilst the terms “mixture” or “reaction mixture” may be used interchangeably with the term “medium” in the present disclosure (especially as it relates to a “one pot” reaction), it should be noted that growing the cells to create a sufficient biomass requires a cell culture/fermentation medium but a medium is not required for the bioconversion step as a reaction buffer will suffice at a suitable pH.

The bioconversion methods of the present disclosure are carried out under conditions of time, temperature, pH and solubilizing agent to provide for conversion of the homofarnesol or bishomofarnesol feedstock (e.g. comprising EEH or bisEEH) to (−)-Ambrox or Ambra oxide.

The pH of the reaction mixture may be in the range of 4-8, preferably, 5 to 6.5, more preferably 4.8-6.0 for the SHC/HAC enzyme variants and in the range of from about pH 5.0 to about pH 7.0 for the wild-type SHC/HAC enzymes and can be maintained by the addition of buffers to the reaction mixture. Exemplary buffers for this purpose include but are not limited to a citric acid buffer, a phosphate buffer, an acetic acid buffer and/or a succinic acid buffer.

The optimum pH range for the wild type SHC/HAC enzymes or the SHC/HAC enzyme variants may from about 5 to about 7, from about 5 to about 6.5, or from about 5 to about 6.

The preferred temperature is between from about 15° C. and about 60° C., for example from about 15° C. to about 50° C. or from about 15° C. to about 45° C. or from about 30° C. to about 60° C. or from about 30° C. to about 40° C. or from about 35 C to about 40° C. or or from about 40° C. to about 45° C. from about 40° C. to about 50° C. The temperature can be kept constant or can be altered during the bioconversion process.

The optimum temperature range for the wild type SHC/HAC enzymes or the SHC/HAC enzyme variants described herein may, for example, be from about 25° C. to about 55° C., from about 25° C. to about 50° C., from about 30° C. to about 55° C., or from about 30° C. to about 50° C.

The Applicant has demonstrated that it may be useful to include a solubilizing agent (e.g. a surfactant, detergent, solubility enhancer, water miscible organic solvent and the like) in the bioconversion reaction.

As used herein, the term “surfactant” means a component that lowers the surface tension (or interfacial tension) between two liquids or between a liquid and a solid. Surfactants may act as detergents, wetting agents, emulsifiers, foaming agents, and dispersants. Examples of surfactants include but are not limited to Triton X-100, Tween 80, taurodeoxycholate, Sodium taurodeoxycholate, Sodium dodecyl sulfate (SDS), and/or sodium lauryl sulfate (SLS).

Whilst Triton X-100 may be used to partially purify the SHC/HAC enzyme or enzyme variant (in soluble or membrane fraction/suspension form), it may also be used in the bioconversion reaction (see for example the disclosure in Seitz (2012 PhD thesis as cited above) as well as the disclosure in Neumann and Simon (1986—as cited above) and JP2009060799. However, surprisingly, the Applicant selected and identified SDS as a particularly useful solubilizing agent from a long list of other less useful solubilizing agents. In particular, the Applicant identified SDS as a remarkably better solubilizing agent than e.g. Triton X-100 in terms of reaction velocity and yield for the homofarnesol to (−)-Ambrox bioconversion reaction (when EEH is used at both 4 g/l and 125 g/l. For at least one SHC/HAC derivative enzyme, that maximal homofarnesol to (−)-Ambrox bioconversion activity with Triton X-100 (at a concentration range of about 0.005% to 0.48%) in the reaction may be only around 20% of the activity obtained with SDS (at a concentration of about 0.07%) with EEH at 4 g/l and cells at an OD_{650 nm} of 10.

Without wishing to be bound by theory, the use of SDS with recombinant microbial host cells may be advantageous as the SDS may interact advantageously with the host cell membrane in order to make the SHC/HAC enzyme or enzyme variant (which is a membrane bound enzyme) more accessible to the homofarnesol substrate. In addition, the inclusion of SDS at a suitable level in the reaction mixture may improve the properties of the emulsion (homofarnesol in water) and/or improve the access of the homofarnesol substrate to the SHC enzyme within the host cell while at the same time preventing the disruption (e.g. denaturation of the SHC (WT or SHC/HAC variant) enzyme). The concentration of the solubilising agent (e.g. SDS) used in the bioconversion reaction is influenced by the biomass amount and the substrate (EEH) concentration. That is, there is a degree of interdependency between the solubilising agent (e.g. SDS) concentration, the biomass amount and the substrate (EEH) concentration. By way of example, as the concentration of homofarnesol substrate increases, sufficient amounts of biocatalyst and solubilising agent (e.g. SDS) are required for an efficient bioconversion reaction to take place. If, for example, the solubilising agent (e.g. SDS) concentration is too low, a suboptimal homofarnesol conversion may be observed. On the other hand, if, for example, the solubilising agent (e.g. SDS) concentration is too high, then there may be a risk that the biocatalyst is affected through either the disruption of the intact microbial cell and/or denaturation/inactivation of the SHC/HAC enzyme or enzyme variant. The selection of a suitable concentration of SDS in the context of the biomass amount and, substrate (EEH) concentration is within the knowledge of the Skilled Person. By way of example, a predictive model can be developed by the Skilled Person to determine the suitable SDS, substrate (EEH) and biomass concentrations. SDS in the range of 0.010-0.075% may be appropriate when 4 g/l EEH and biocatalyst to an OD of 10.0 (650 nm) are used. When 125 g/l EEH is used with 2× the wet weight of biomass, an adjusted SDS concentration (1.3%) may be appropriate. However, an investigation of the percent EEH conversion to (−)-Ambrox using different SDS/cells ratio values indicated that the correct selection of the ratio of biocatalyst, homofarnesol substrate and solubilising agent (e.g. SDS) facilitates the development of a robust bioconversion reaction system which demonstrates a degree of tolerance to a range of SDS concentrations and pH ranges.

The temperature of the bioconversion reaction for a WT SHC enzyme (eg. AacSHC) or SHC/HAC variant may be from about 25° C. to about 55° C., from about 25° C. to about 50° C., from about 30° C. to about 55° C., or from about 30° C. to about 50° cm from about 30° C. to about 60° C., for example from about 45° C. to about 60° C., for example from about 50° C. to about 60° C., for example about 55° C.

The pH range of the bioconversion reaction for a WT SHC enzyme (eg. AacSHC) or SHC/HAC variant may be from about 5.0 to 7.0, from about 5 to about 6.5, or from about 5 to about 6, more preferably from about 5.6 to about 6.2, even more preferably about 6.0.

The temperature of the bioconversion reaction for a SHC/HAC enzyme variant may be about 30° C. to about 55° C., for example from about 40° C. to about 50° C., for example about 45° C.

The pH of the bioconversion reaction for a SHC/HAC enzyme variant may be about 4.8-6.4, preferably about 5.2-6.0.

Preferably the solubilising agent used in the bioconversion reaction is SDS. The SDS concentration used in the bioconversion reaction for the WT SHC enzyme (e.g. AacSHC) may be in the range of about 0.010-0.075%, preferably about 0.030% when EEH at about 4 g/l and cells at an OD_{650 nm} of 10 is used.

The SDS concentration used in the bioconversion reaction for the SHC/HAC enzyme variant may be in the range of about 0.005 w/w % to about 0.04 w/w %, from about 0.010 w/w % to about 0.10 w/w %, from about 0.0025-0.090%, preferably about 0.050% when EEH at about 4 g/l and cells at an OD_{650 nm} of 10 is used.

The biocatalyst may be loaded to the reaction to an OD of about 10.0 (650 nm) when the reaction is loaded with homofamesol at an EEH concentration of about 4 g/l EEH.

The [SDS]/[cells] ratio may be in the range of about, 10:1-20:1, preferably about 15:1-18:1, preferably about 16:1 when the ratio of biocatalyst to EEH homofamesol is about 2:1 in the bioconversion reaction.

The SDS concentration in the bioconversion reaction for a SHC variant enzyme may be in the range of about 1-2%, preferably in the range of about 1.4-1.7%, even more preferably about 1.5% when the homofamesol concentration is about 125 g/l EEH and the biocatalyst concentration is 250 g/l (corresponding to an OD of about 175 (650 nm)).

The ratio of biocatalyst to EEH homofamesol substrate may be in the range of about 0.5:1-2:1, in some embodiments 2:1, preferably about 1:1 or 0.5:1.

The weight ratio of the recombinant host cell producing the SHC/HAC enzyme variant to EEH or a mixture of isomers of homofarnesol comprising EEH in an EE:EZ isomer mixture in a weight ratio of 80:20 is about 2:1, 1:1 or about 0.5:1 or about 0.1:1.

The weight ratio of the recombinant host cell producing the SHC/HAC enzyme variant to EEH or a mixture of isomers of homofarnesol comprising EEH in an EE:EZ isomer mixture in a weight ratio of EEH:EZH is about 100:00; 99:01; 98:02; 97:03; 96:04; 95:05; 94:06; 93:07; 92:08; 91:09; 90:10; 89:11; 88:12; 87:13; 86:14; 85:15; 84:16; 83:17; 82:18; 81:19; 80:20; 79:21; 78:22; 77:23; 76:24; 75:25; 74:26; 73:27; 72:28; 71:29; 70:30; 69:31; 68:32; 67:33; 66:34; 65:35; 64:36; 63:37; 62:38:61:39; 60:40; 59:41; 58:42; 57:43; 56:44; 55:45:54:46; 53:47:52:48; 51:49; or about 50:50 is about 2:1, 1:1 or about 0.5:1 or about 0.1:1.

The weight ratio of the recombinant host cell producing the SHC/HAC enzyme variant to EEH or a mixture of isomers of homofarnesol comprising EEH in an EE:EZ isomer mixture in a weight ratio of from about 50:50 to about 100:00 or from about 50:50 to about 99:01 or from about 60:40 to about 99:1 or from about 70:30 to about 95:5 or from about 80:20 to about 95:5. preferably 80:20 is about 2:1, 1:1 or about 0.5:1 or about 0.1:1.

In an embodiment, a cell (or biocatalyst) to EEH ratio of 0.5 or 0.4 or 0.3 or 0.2 or 0.1 is used. In an embodiment, when such a cell (or biocatalyst) to EEH ratio is used, the EEH conversion is of at least 50%, 60%, 70%, 80%, 90%, 95% or 98%. In an embodiment, such a cell (or biocatalyst) to EEH ratio is used even when 100 or 150 or 200 or even 250 g/l EEH is present. In an embodiment when such cell (or biocatalyst) to EEH ratio is used and such concentration of EEH is used a conversion of up to 4.5 or 5.0 or 5.5 or even 5.9 or 6.0 g of EEH is converted per gram of cells (or biocatalyst). Such results have been obtained in the experimental part (see example 9). In the context of the reference to the cell (biocatalyst) to EEH ratio, EEH may be present in a mixture with other isomer of homofarnesol as described herein. We refer to the total concentration of EEH present even if other isomer of homofarnesol may be present.

As Example 9 demonstrates, under certain reaction conditions using the SHC variants disclosed and described herein, the [cells]:[EEH] ratio was lowered further down to values of 0.3, 0.2, and 0.1. The reaction time was extended to approx. 200 h to possibly allow for reaching a plateau in E,E-Homofarnesol conversion. Approx. 98% EEH conversion was obtained with a [cells]:[EEH] ratio of 0.3, approx. 84% EEH conversion with a [cells]:[EEH] ratio of 0.2, and approx. 59% with a [cells]:[EEH] of 0.1. The plateau reached in the reaction with a [cells]:[EEH] ratio of 0.1 indicated the possibility of converting approx. 5.9 g of E,E-Homofarnesol per gram of cells under the reaction conditions applied which represents about a three fold improvement over reference bioconversion reactions.

Furthermore in Example 17, it was demonstrated that using 450 g/l EEH and a [cells]:[EEH] ratio of 0.4, full EEH conversion was obtained in 72 h. Full conversion was obtained in 24 h with 250 g/l EEH at [cells]:[EEH] ratio of 1.0. This indicated the possibility for full conversion of up to 450 g/l EEH in 3 days using an appropriate amount of cells, and that this was possible with a [cells]:[EEH] ratio at least as low as 0.4 under the conditions tested.

Therefore, in an embodiment, a process is provided wherein the weight ratio of the recombinant host cell producing the SHC/HAC enzyme variant to EEH or a mixture of isomers of homofarnesol comprising EEH in an EE:EZ isomer mixture is 0.4 and the concentration of EEH is 450 g/l.

Therefore, in an embodiment, a process is provided wherein the weight ratio of the recombinant host cell producing the SHC/HAC enzyme variant to EEH or a mixture of isomers of homofarnesol comprising EEH in an EE:EZ isomer mixture is in a range of 0.1-0.5, preferably 0.4 and the concentration of EEH is in the range of 250 g/l to 650 g/l, preferably 450 g/l.

In another embodiment, a process is provided wherein the weight ratio of the recombinant host cell producing the SHC/HAC enzyme variant to EEH or a mixture of isomers of homofarnesol comprising EEH in an EE:EZ isomer mixture is 1 and the concentration of EEH is 250 g/l.

This improvement can be attributed to an increase in total turnover number when certain SHC mutations (eg P2 mutations) are introduced into a reference SHC enzyme (eg SHC #65). This increased total turnover number may be the result of an increased catalytic potential or an increased stability of the new SHC variant (eg SHC #65 P2 enzyme variant), or a combination of the two. An increased stability may be the result of the optimal reaction conditions for the new SHC variant (eg SHC #65 P2) with significantly lower temperature and SDS concentration.

The optimum temperature for the SHC/HAC enzyme variants may, for example, be equal to or greater than about 35° C. For example, the optimum temperature for the SHC/HAC enzyme variants may range from about 40° C. to about 50° C., for example from about 42° C. to about 48° C. or from about 44° C. to about 46° C. For example, the optimum temperature of the SHC/HAC enzyme variants may be about 45° C. The processes for making (−)-Ambrox or Ambra oxide disclosed herein may be carried out at the optimum temperature of the SHC/HAC enzyme variant.

The optimum pH for the SHC/HAC enzyme variants may, for example, be equal to or greater than about 5.4. For example, the optimum pH for the SHC/HAC enzyme variants may range from about 5.2 to about 6.0, for example from about 5.4 to about 5.8, for example from about 5.6 to about 5.8. For example, the optimum pH of the SHC/HAC enzyme variants may be about 5.6 or about 5.8. The process for making (−)-Ambrox or Ambra oxide disclosed herein may be carried out at the optimum pH of the SHC/HAC enzyme variant.

The optimum concentration of sodium dodecyl sulfate (SDS) in the reaction medium of the process for making (−)-Ambrox or Ambra oxide disclosed herein may, for example, be from about 0.005 w/w % to about 0.04 w/w %, from about 0.010 w/w % to about 0.10 w/w %. For example, the optimum concentration of SDS may be from about 0.040 w/w % to about 0.080 w/w %, for example about 0.050 w/w % when the substrate (e.g. EEH or BisEEH) is used at 4 g/l with cells to an OD_{650 nm} of 10 The process for making (−)-Ambrox or Ambra oxide disclosed herein may be carried out using the optimum concentration of SDS described herein. The optimum concentration of sodium dodecyl sulfate (SDS) in the reaction medium of the processes for making (−)-Ambrox or Ambra oxide disclosed herein may, for example, be from about 1.0 w/w % to about 1.5 w/w % when the substrate (e.g. EEH or BisEEH) is used at 125 g/l with 250 g/l of cells. For example, the optimum concentration of SDS may be from about 1.2 w/w % to about 1.4 w/w %, for example about 1.3 w/w % when the substrate (e.g. EEH or BisEEH) is used at 125 g/l with 250 g/l of cells or about 0.65 w/w % when the substrate (e.g. EEH or BisEEH) is used at 125 g/l with 125 g/l of cells.

The processes for making (−)-Ambrox or Ambra oxide disclosed herein may be carried out within the optimum temperature range or at the optimum temperature and/or within the optimum pH range or at the optimum pH and/or within the SDS optimum concentration range or at the optimum SDS concentration for the specific enzyme used, as set out in Table 15 in the Examples below.

In some embodiments, (−)-Ambrox is produced using a biocatalyst to which the homofamesol substrate is added.

In some embodiments, Ambra oxide is produced using a biocatalyst to which the bishomofamesol substrate is added.

It is possible to add the substrate by feeding using known means (e.g. peristaltic pump, infusion syringe and the like). Homofamesol and bishomofarnesol may be oil soluble compounds and provided in an oil format. Given that the biocatalyst (microbial cells such as intact recombinant whole cell and/or cell debris and/or immobilised enzyme) is present in an aqueous phase, the bioconversion reaction may be regarded as a three phase system (comprising an aqueous phase, a solid phase and an oil phase) when homofarnesol or bishomofarnesol is added to the bioconversion reaction mixture. This is the case even when SDS is present. By way of clarification, when a soluble WT SHC or a SHC/HAC enzyme variant is used as a biocatalyst, this is considered a two phase system.

A fermenter may be used to grow recombinant host cells expressing the SHC/HAC enzyme or enzyme variant gene and producing active SHC/HAC enzymes or enzyme variants to a sufficient biomass concentration suitable for use as a biocatalyst in the same fermenter vessel which is used to convert the homofarnesol source to (−)-Ambrox, for example in admixture with one or more of the by-products (II), (IV) and/or (III) or bishomofarnesol source to Ambra oxide, for example in admixture with one or more of the by-products (XI), (XII) and/or (XIII). Within the application, each molecule may be called a compound or a by-product depending on the context in which reference is made to said molecule. Usually, the wording “by-product” is used when said molecule is not desired but can be accepted under certain conditions in the end product. If a by-product is included in an end product (eg either alone or as part of a composition), it may be referred to as a compound rather than a by-product). By way of example, a by-product may be acceptable in an end product if does not impact or substantially impact on the olfactory profile of the end product or the composition comprising the by-product.

In an embodiment, the SHC/HAC variants disclosed herein are so selective for an EEH substrate isomer and so selective for the production of (−)-Ambrox that using pure EEH as the sole substrate isomer allows for the production of (−)-Ambrox and undetectable by-product (IV), even though no further downstream processing (such as filtration/selective crystallization/distillation) is carried out.

In an embodiment, the SHC/HAC variants disclosed herein are so selective for an EEH substrate isomer and so selective for the production of (−)-Ambrox that using pure EEH as the sole substrate isomer allows for the production of (−)-Ambrox. Moreover, by-product (IV) is undetectable, even though no further downstream processing (such as filtration/selective crystallization/distillation) is carried out.

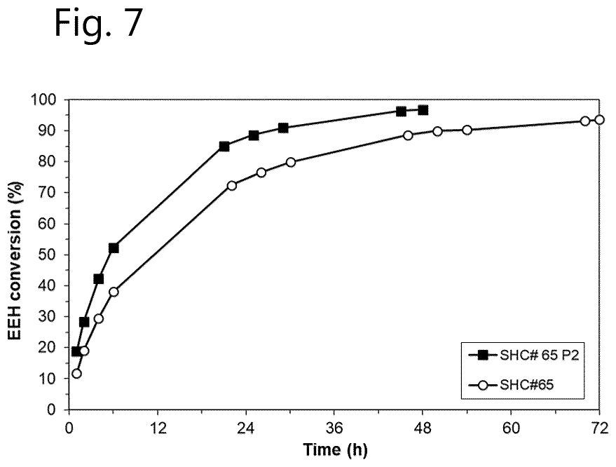

In an embodiment, the SHC/HAC variants disclosed herein are so selective for an EEH substrate isomer and so selective for the production of (−)-Ambrox that using a mixture of homofarnesol substrate isomers comprising at least two distinct substrate isomers being the EEH and the EZH isomers and being selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)] allows for the production of only (−)-Ambrox and by-product (III). In such embodiment, by-products (II) and (IV) are not detectable, even though no further downstream processing (such as filtration/selective crystallization/distillation) is carried out. This has been demonstrated in example 7 (FIG. 6).

In an embodiment, the mixture comprises EEH and EZH. In an embodiment, the mixture is [(3E,7E), (3Z,7E)] also designated [EE:EZ] or EEH:EZH. In an embodiment, the ratio of EEH:EZH within this mixture may range from about 50:50 to about 99:01 or from about 60:40 to about 99:1 or from about 70:30 to about 95:5 or from about 80:20 to about 95:5.

In an embodiment, the SHC/HAC variants disclosed herein are so selective for an EEH substrate isomer and so selective for the production of (−)-Ambrox that using a mixture of homofarnesol substrate isomers comprising at least two distinct substrate isomers being the EEH and the EZH isomers and being selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)] allows the production of (-only (−)-Ambrox and at least by-product (II).

In an embodiment, the mixture comprises EEH and EZH. In an embodiment, the mixture is [(3E,7E), (3Z,7E)] also designated [EE:EZ] or EEH:EZH. In an embodiment, the ratio of EEH:EZH within this mixture may range from about 50:50 to about 99:01 or from about 60:40 to about 99:1 or from about 70:30 to about 95:5 or from about 80:20 to about 95:5.

In an embodiment, the SHC/HAC variants disclosed herein are so selective for EEH and so selective for the production of (−)-Ambrox that using a mixture of homofarnesol substrate isomers comprising at least two distinct substrate isomers being the EEH and the EZH isomers and being selected from one of more of the following mixtures: [(3Z,7Z), (3E,7Z), (3Z,7E) and (3E,7E)], [(3Z,7E) and (3E,7E)], [(3Z,7E), (3E,7Z)] and/or [(3E,7E) and (3E,7Z)] allows the production of only (−)-Ambrox and at least by-product Ill but wherein by-products (II) and (IV) are not detected.

In an embodiment, the mixture comprises EEH and EZH. In an embodiment, the mixture is [(3E,7E), (3Z,7E)] also designated [EE:EZ] or EEH:EZH. In an embodiment, the ratio of EEH:EZH within this mixture may range from about 50:50 to about 99:01 or from about 60:40 to about 99:1 or from about 70:30 to about 95:5 or from about 80:20 to about 95:5.

The Skilled Person will understand that higher cumulative production titers can be achieved by implementing a continuous process, such as product removal, substrate feed, and biomass addition or (partial) replacement. Preferably the bioconversion of EEH into (−)-Ambrox or BisEEH to Ambra oxide in the presence of a recombinant host cell comprising a SHC/HAC enzyme or enzyme variant generates an (−)-Ambrox or Ambra oxide yield of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, given in mol percent and based on the mols of EEH or BisEEH employed; especially preferably, the yield is between 5 and 100, 10 and 100, and 100, 25 and 100, 30 and 100, 35 and 100, in particular between 40 and 100, 45 and 100, 50 and 100, 60 and 100, 70 and 100 mol percent.

The activity of the SHC/HAC enzyme or enzyme variant is defined via the reaction rate (amount of product/(amount of product+amount of remaining starting material))×100) in mol percent.

Preferably, the bioconversion of EEH into (−)-Ambrox or BisEEH into Ambra oxide in the presence of a SHC/HAC enzyme or enzyme variant produces an (−)-Ambrox or Ambra oxide yield of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, given in mol percent and based on the mols of EEH or BisEEH employed; especially preferably, the yield is between 5 and 100, 10 and 100, 20 and 100, 25 and 100, 30 and 100, 35 and 100, in particular between 40 and 100, 45 and 100, 50 and 100, 60 and 100, 70 and 100.

In a preferred embodiment of the invention, the yield and/or the reaction rate are determined over a defined time period of, for example, 4, 6, 8, 10, 12, 16, 20, 24, 36 or 48 hours, during which EEH is converted into (−)-Ambrox or BisEEH is converted into Ambra oxide by a recombinant host cell comprising a nucleotide sequence encoding a SHC/HAC enzyme or enzyme variant. In a further variant, the reaction is carried out under precisely defined conditions of, for example, 25° C., 30° C., 40° C., 50° C. or 60° C. In particular, the yield and/or the reaction rate are determined by carrying out the reaction of converting EEH into (−)-Ambrox or BisEEH into Ambra oxide by the SHC/HAC enzymes or enzyme variants according to the invention at 35° C. over a period of 24-72 hours.

In a further embodiment of the present invention, a recombinant host cell comprising a nucleotide sequence encoding a SHC/HAC enzyme variant is characterized in that it shows a 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48- , 49-, 50-, 51-, 52-, 53-, 54-, 55-, 56-, 57-, 58-, 59-, 60-, 61-, 62-, 63-, 64-, 65-, 66-, 67-, 68-, 69- , 70-, 71-, 72-, 73-, 74-, 75-, 76-, 77-, 78-, 79-, 80-, 81-, 82-, 83-, 84-, 85-, 86-, 87-, 88-, 89-, 90- , 91-, 92-, 93-, 94-, 95-, 96-, 97-, 98-, 99-, 100-, 200-, 500-, 1000-fold or higher yield and/or reaction rates in the reaction of homofamesol to give (−)-Ambrox or in the reaction of bishomofarnesol to give Ambra oxide in comparison with the WT SHC or SHC/HAC derivative enzyme under the same conditions. Here, the term condition relates to reaction conditions such as substrate concentration, enzyme concentration, reaction period and/or temperature.

The successful development of a bioconversion process for making (−)-Ambrox from homofamesol in a recombinant strain of E. coli comprising a nucleotide sequence encoding a WT/reference SHC or a SHC/HAC derivative can offer a low cost and industrially economical process for (−)-Ambrox production.

The successful development of a bioconversion process for making Ambra oxide from bishomofamesol in a recombinant strain of E. coli comprising a nucleotide sequence encoding a WT/reference SHC or a SHC/HAC derivative can offer a low cost and industrially economical process for Ambra oxide production.

### Ambrox and Uses Thereof

There is further provided herein the reaction products made by the processes described herein. The reaction products may, for example, comprise, consist essentially of consist of (−)-Ambrox and one or more further compounds, for example one or more of a compound of formula (II), a compound of formula (III) and a compound of formula (IV).

As used herein, the term “Ambrox” includes (−)-Ambrox of formula (I) below as well as (−)-Ambrox in isomerically pure form or in a mixture with one or more of the following molecules of formula (II), (III), and/or (IV),

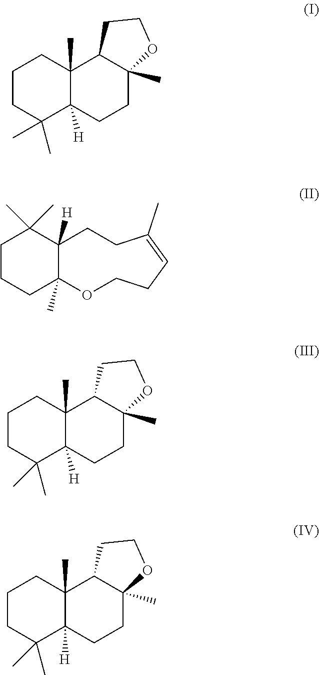 [SMILES: CC1(C)CCC[C@]2(C)[C@H]3CCO[C@]3(C)CC[C@@H]12 ; C/C1=C/CCO[C@@]2(C)CCCC(C)(C)[C@@H]2CC1 ; CC1(C)CCC[C@@]2(C)[C@H]1CC[C@@]1(C)OCC[C@@H]21 ; CC1(C)CCC[C@@]2(C)[C@H]1CC[C@]1(C)OCC[C@@H]21]

The nomenclature for the reaction products of formulae (I), (II), (III), and (IV) is set out below.

TABLE 13
Nomenclature for the reaction products of formulae (I), (II), (III) and (IV).

| Compound | Description | Name and Structure |
|---|---|---|
| (I) | (-)-Ambrox | 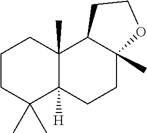 [SMILES: CC1(C)CCC[C@]2(C)[C@H]3CCO[C@]3(C)CC[C@@H]12] |
| | | (3aR,5aS,9aS,9bR)-3a,6,6,9a- |
| | | tetramethyldodecahydronaphtho[2,1-b]furan |
| (II) | Macrocycle | 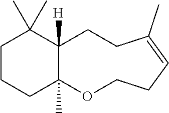 [SMILES: C/C1=C/CCO[C@@]2(C)CCCC(C)(C)[C@@H]2CC1] |
| | | (7aS,11aS,Z)-5,8,8,11a-tetramethyl-2,3,6,7,7a,8,9,10,11,11a- |
| | | decahydrobenzo[b]oxonine |
| (III) | 9b-epi-Ambrox | 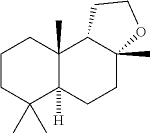 [SMILES: CC1(C)CCC[C@@]2(C)[C@H]1CC[C@@]1(C)OCC[C@@H]21] |
| | | (3aR,5aS,9aS,9bS)-3a,6,6,9a-tetramethyldodeca- |
| | | hydronaphtho[2,1-b]furan |
| (IV) | Escher et al (1990) | 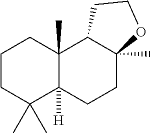 [SMILES: CC1(C)CCC[C@@]2(C)[C@H]1CC[C@]1(C)OCC[C@@H]21] |
| | | (3aS,5aS,9aS,9bS)-3a,6,6,9a-tetramethyldodeca- |
| | | hydronaphtho[2,1-6]furan |

(−)-Ambrox is known commercially as Ambrox (Firmenich), Ambroxan (Henkel), Ambrofix (Givaudan), Amberlyn (Quest), Cetalox Laevo (Firmenich), Ambermor (Aromor) and/or Norambrenolide Ether (Pacific).

(−)-Ambrox is an industrially important aroma compound and has been used in the fragrance industry for a long time. The special desirable sensory benefits from (−)-Ambrox come from the (−) isomer rather than the (+) one. The odour of the (−) isomer is described as musk-like, woody, warm or ambery whereas the (+)-Ambrox enantiomer has a relatively weak odour note. The odour and odour thresholds for Ambrox like products are also different. While various (−)-Ambrox enriched materials are available commercially, it is desirable to produce highly enriched (−)-Ambrox materials, ideally pure (−)-Ambrox. The processes described herein may make (−)-Ambrox of formula (II) alone or in a mixture with by-products such as the compounds shown in formulae (II), (III) and/or (IV) above.

(−)-Ambrox can be produced from sclareolide according to the production process as described below. Sclareol is a product extracted from the natural plant clary sage. However, because a natural starting material is used in this process, there are potential problems in that it involves a multistage reaction, its operation is circuitous, the quantity and stability of supply of starting material may not always be satisfactory, and the reaction may not be environmentally friendly because an oxidizing agent such as chromic acid or a permanganate is used in the step of (+)-sclareol oxidative degradation.

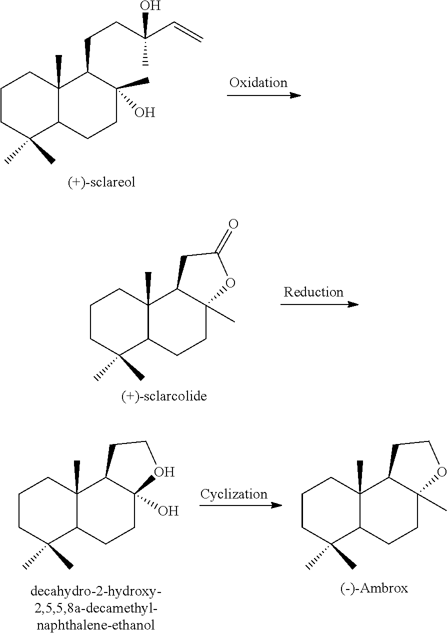

(−)-Ambrox may also be synthesized from homofarnesol using different routes. By way of example, homofarnesol can be obtained by brominating, cyanating, and hydrolysing nerolidol to give homofarnesylic acid, followed by reduction. Alternatively, homofarnesol may be obtained from farnesol, farnesylchloride, beta-farnesene or other substrates.

The processes described herein may make (−)-Ambrox of formula (II) alone or in a mixture with by-products such as the compound shown in formulae (II), (III) and/or (IV). For example, other stereoisomers of formula (I) may be made by the processes described herein. As explained earlier herein, in an embodiment, when only EE homofarnesol is present the process described herein allows the production of (−)-Ambrox with no by-product (IV) or undetectable by-product (IV) indicating a high substrate specificity and product selectivity for the SHC/HAC enzyme variant described herein.

There is therefore provided herein a compound of formula (I) or a composition comprising a compound of formula (I) obtained by or obtainable by the processes described herein, including all embodiments thereof.

In certain embodiments, not all of the homofarnesol (e.g. EEH) is converted to (−)-Ambrox or a by-product of the reaction. Therefore, the compositions described herein, for example the compositions obtained by obtainable by the processes described herein may comprise homofarnesol (e.g. EEH, for example in addition to the compound of formula (I) and/or compounds of formula (II), (III) and/or (IV)). Any remaining homofarnesol may be separated from the other reaction products such that the (−)-Ambrox product does not comprise homofarnesol. In other embodiments, all of the homofarnesol starting material is converted to (−)-Ambrox of formula (I) or a by-product of the reaction by the processes described herein.

Therefore, the compositions described herein may comprise, consist essentially of or consist of one or more of a compound of formula (I), a compound of formula (II), a compound of formula (III), a compound of formula (IV), homofarnesol starting material (e.g. EEH) and other stereoisomers of the compound of formula (I).

For example, the compositions described herein may comprise, consist essentially of or consist of a compound of formula (I) and one or more of a compound of formula (II), a compound of formula (III) and a compound of formula (IV).

For example, the compositions described herein may comprise, consist essentially of or consist of a compound of formula (I) and of a compound of formula (II) and a compound of formula (III).

Therefore, in an embodiment, when only the EE homofarnesol isomer is present in the bioconversion reaction, the compound of formula (IV) is not detectable even though no isolation/purification/downstream processing steps has been carried out.

For example, the compositions described herein may comprise, consist essentially of or consist of a compound of formula (I) and a compound of formula (III).

Therefore, in an embodiment, when the EE/EZ homofarnesol isomers are present in the bioconversion reaction the compound of formula (II) and (IV) are not detectable even though no purification/downstream processing steps had been carried out.

The compositions described herein may therefore comprise equal to or greater than about equal to or greater than about 50 wt % of the compound of formula (II) based on the total weight of the compound of formula (II), the compound of formula (II), the compound of formula (III) and the compound of formula (IV). For example, the compositions described herein may comprise equal to or greater than about 55 wt % or equal to or greater than about 60 wt % or equal to or greater than about 65 wt % or equal to or greater than about 70 wt % or equal to or greater than about 75 wt % or equal to or greater than about 80 wt % or equal to or greater than about 85 wt % or equal to or greater than about 90 wt % or equal to or greater than about 95 wt % of the compound of formula (I) based on the total weight of the compound of formula (II), the compound of formula (II), the compound of formula (III) and the compound of formula (IV). The compositions described herein may, for example, comprise equal to or less than about 100 wt % of the compound of formula (II) based on the total weight of the compound of formula (II), the compound of formula (II), the compound of formula (III) and the compound of formula (IV). For example, the mixture may comprise equal to or less than about 99 wt % or equal to or less than about 98 wt % or equal to or less than about 97 wt % of the compound of formula (II) based on the total weight of compound of formula (II), the compound of formula (II), the compound of formula (III) and the compound of formula (IV). For example, the compositions described herein may comprise from about 50 wt % to about 100 wt % or from about 60 wt % to about 99 wt % or from about 70 wt % to about 98 wt % or from about 80 wt % to about 97 wt % or from about 90 wt % to about 97 wt % of the compound of formula (II) based on the total weight of the compound of formula (II), the compound of formula (II), the compound of formula (III) and the compound of formula (IV).

The weight ratio of the compound of formula (II) to the total weight of the compound of formula (II), the compound of formula (III) and the compound of formula (IV) in the compositions described herein may, for example, range from about 60:40 to about 99:1. For example, the weight ratio of the compound of formula (II) to the total weight of the compound of formula (II), the compound of formula (III) and the compound of formula (IV) may range from about 65:35 to about 99:1 or from about 70:30 to about 99:1 or from about 75:25 to about 99:1 or from about 80:20 to about 99:1 or from about 85:15 to about 99:1 or from about 90:10 to about 99:1 or from about 95:5 to about 99:1. For example, the weight ratio of the compound of formula (II) to the total weight of the compound of formula (II), the compound of formula (III) and the compound of formula (IV) may range from about 65:35 to about 98:2 or from about 70:30 to about 97:3 or from about 75:25 to about 96:4 or from about 80:20 to about 95:5 or from about 85:15 to about 90:10.

The compositions described herein may therefore comprise equal to or greater than about equal to or greater than about 50 wt % of the compound of formula (II) based on the total weight of the compound of formula (I), the compound of formula (II) and the compound of formula (III). For example, the compositions described herein may comprise equal to or greater than about 55 wt % or equal to or greater than about 60 wt % or equal to or greater than about 65 wt % or equal to or greater than about 70 wt % or equal to or greater than about 75 wt % or equal to or greater than about 80 wt % or equal to or greater than about 85 wt % or equal to or greater than about 90 wt % or equal to or greater than about 95 wt % of the compound of formula (II) based on the total weight of the compound of formula (II), the compound of formula (II) and the compound of formula (III). The compositions described herein may, for example, comprise equal to or less than about 100 wt % of the compound of formula (II) based on the total weight of the compound of formula (II), the compound of formula (II) and the compound of formula (III). For example, the mixture may comprise equal to or less than about 99 wt % or equal to or less than about 98 wt % or equal to or less than about 97 wt % of the compound of formula (II) based on the total weight of compound of formula (II), the compound of formula (II) and the compound of formula (III). For example, the compositions described herein may comprise from about 50 wt % to about 100 wt % or from about 60 wt % to about 99 wt % or from about 70 wt % to about 98 wt % or from about 80 wt % to about 97 wt % or from about 90 wt % to about 97 wt % of the compound of formula (II) based on the total weight of the compound of formula (II), the compound of formula (II) and the compound of formula (III).

The weight ratio of the compound of formula (II) to the total weight of the compound of formula (II) and the compound of formula (III) in the compositions described herein may, for example, range from about 60:40 to about 99:1. For example, the weight ratio of the compound of formula (II) to the total weight of the compound of formula (II) and the compound of formula (III) may range from about 65:35 to about 99:1 or from about 70:30 to about 99:1 or from about 75:25 to about 99:1 or from about 80:20 to about 99:1 or from about 85:15 to about 99:1 or from about 90:10 to about 99:1 or from about 95:5 to about 99:1. For example, the weight ratio of the compound of formula (I) to the total weight of the compound of formula (II) and the compound of formula (III) may range from about 65:35 to about 98:2 or from about 70:30 to about 97:3 or from about 75:25 to about 96:4 or from about 80:20 to about 95:5 or from about 85:15 to about 90:10.

The compositions described herein may therefore comprise equal to or greater than about equal to or greater than about 50 wt % of the compound of formula (II) based on the total weight of the compound of formula (II) and the compound of formula (III). For example, the compositions described herein may comprise equal to or greater than about 55 wt % or equal to or greater than about 60 wt % or equal to or greater than about 65 wt % or equal to or greater than about 70 wt % or equal to or greater than about 75 wt % or equal to or greater than about 80 wt % or equal to or greater than about 85 wt % or equal to or greater than about 90 wt % or equal to or greater than about 95 wt % of the compound of formula (II) based on the total weight of the compound of formula (I) and the compound of formula (III). The compositions described herein may, for example, comprise equal to or less than about 100 wt % of the compound of formula (II) based on the total weight of the compound of formula (II) and the compound of formula (III). For example, the mixture may comprise equal to or less than about 99 wt % or equal to or less than about 98 wt % or equal to or less than about 97 wt % of the compound of formula (II) based on the total weight of compound of formula (II) and the compound of formula (III). For example, the compositions described herein may comprise from about 50 wt % to about 100 wt % or from about 60 wt % to about 99 wt % or from about 70 wt % to about 98 wt % or from about 80 wt % to about 97 wt % or from about 90 wt % to about 97 wt % of the compound of formula (II) based on the total weight of the compound of formula (II) and the compound of formula (III).

The weight ratio of the compound of formula (II) to the total weight of the compound of formula (III) in the compositions described herein may, for example, range from about 60:40 to about 99:1.

For example, the weight ratio of the compound of formula (II) to the total weight of the compound of formula (III) may range from about 65:35 to about 99:1 or from about 70:30 to about 99:1 or from about 75:25 to about 99:1 or from about 80:20 to about 99:1 or from about 85:15 to about 99:1 or from about 90:10 to about 99:1 or from about 95:5 to about 99:1. For example, the weight ratio of the compound of formula (II) to the total weight of the compound of formula (III) may range from about 65:35 to about 98:2 or from about 70:30 to about 97:3 or from about 75:25 to about 96:4 or from about 80:20 to about 95:5 or from about 85:15 to about 90:10.

The weight ratio of the compound of formula (II) to homofarnesol (e.g. EEH) in the compositions described herein may, for example, range from about 90:10 to about 100:0. For example, the weight ratio of the compound of formula (II) to homofarnesol (e.g. EEH) in the compositions described herein may range from about 92:8 to about 100:0 or from about 94:6 to about 100:0 or from about 95:5 to about 100:0 or from about 96:4 to about 99.5:0.5 or from about 97:3 to about 99.0:1.0 or from about 98:2 to about 99.0:1.0.

The amount of the compound of formula (II), the compound of formula (II), the compound of formula (III) and the compound of formula (IV) in a mixture of stereoisomers may, for example, be quantified by gas chromatography and/or identified by NMR spectroscopy.

(−)-Ambrox as synthesized by the processes described herein (e.g. using SHC/HAC enzymes or variants thereof and optionally recombinant host cells) may, for example, be in a solid form. The solid form may be amorphous form or in crystalline form.

The (−)-Ambrox produced by the methods described herein (e.g. using SHC/HAC enzymes or variants thereof and optionally recombinant host cells) may be isolated by steam extraction/distillation or organic solvent extraction using a non-water miscible solvent (to separate the reaction products and unreacted substrate from the biocatalyst which stays in the aqueous phase) followed by subsequent evaporation of the solvent to obtain a crude reaction product as determined by gas chromatographic (GC) analysis. The steam extraction/distillation and organic solvent extraction methods are known to those skilled in the art.

(−)-Ambrox is present in the solid phase as crystals or in amorphous form and can be separated from the remaining solid phase (cell material or debris thereof) and the liquid phase (of the reaction mixture (eg unreacted homofarnesol and/or by products (II and/or Ill and/or IV) and/or reaction buffer) by means of filtration (eg using a belt filter or press filters or any filter of appropriate mesh size). Alternatively, the solid phase of the reaction mixture that may comprise (−)-Ambrox can be separated from the liquid phase by, for example, centrifugation.

(−)-Ambrox may be extracted from the solid phase (eg cell material, debris and (−)-Ambrox crystals) using a water miscible solvent (for example ethanol) or a non-water miscible solvent (for example toluene). Alternatively, the resulting (−)-Ambrox may be extracted from the solid phase of the reaction mixture using a mixture of solvents (eg water miscible and non water miscible solvents).

(−)-Ambrox can also be extracted from the whole reaction mixture where it is extracted with a non-water miscible solvent (for example toluene).

Examples of suitable water miscible and non-water miscible organic solvents suitable for use in the extraction and/or selective crystallization of (−)-Ambrox include but are not limited to aliphatic hydrocarbons, preferably those having 5 to 8 carbon atoms, such as pentane, cyclopentane, hexane, cyclohexane, heptane, octane or cyclooctane, halogenated aliphatic hydrocarbons, preferably those having one or two carbon atoms, such as dichloromethane, chloroform, carbon tetrachloride, dichloroethane or tetrachloroethane, aromatic hydrocarbons, such as benzene, toluene, the xylenes, chlorobenzene or dichlorobenzene, aliphatic acyclic and cyclic ethers or alcohols, preferably those having 4 to 8 carbon atoms, such as ethanol, isopropanol, diethyl ether, methyl tert.-butyl ether, ethyl tert.-butyl ether, dipropyl ether, diisopropyl ether, dibutyl ether, tetrahydrofuran or esters such as ethyl acetate or n-butyl acetate or ketones such as methyl isobutyl ketone or dioxane or mixtures of these. The solvents which are especially preferably used are the abovementioned heptane, Methyl tert-butyl ether (also known as MTBE, tert-butyl methyl ether, tertiary butyl methyl ether and tBME), diisopropyl ether, tetrahydrofuran, ethyl acetate and/or mixtures thereof. Preferably, a water miscible solvent such as ethanol is used for the extraction of (−)-Ambrox from the solid phase of the reaction mixture. The use of ethanol is advantageous because it is easy to handle, it is non-toxic and it is environmentally friendly.

By way of example, the resulting (−)-Ambrox may be extracted from the whole reaction mixture using an organic solvent such as a non-water miscible solvent (for example toluene). Alternatively, the resulting (−)-Ambrox may be extracted from the solid phase of the reaction mixture (obtained by, for example, centrifugation or filtration) using a water miscible solvent (for example ethanol) or a non-water miscible solvent (for example toluene). Alternatively, the resulting (−)-Ambrox may be extracted from the solid phase of the reaction mixture using a mixture of solvents. By way of further example, (−)-Ambrox is present in the solid phase as crystals or in amorphous form and can be separated from the remaining solid phase (cell material or debris thereof) and the liquid phase also by means of filtration. By way of further example, at a temperature above the melting point of (−)-Ambrox (around. 75° C.), the (−)-Ambrox may form an oil layer on top of aqueous phase, which oil layer can be removed and collected. In order to ensure a complete recovery of (−)-Ambrox after the oil layer is removed, an organic solvent may be added to the aqueous phase containing the biomass in order to extract any residual (−)-Ambrox contained in, or on or about the biomass. The organic layer can be combined with the oil layer, before the whole is further processed to isolate and purify (−)-Ambrox.

The (−)-Ambrox may be further selectively crystallised to remove by-products (II), (IV) and (III) and any unreacted homofarnesol substrate from the final (−)-Ambrox product. The term “selective crystallization” refers to a process step whereby (−)-Ambrox is caused to crystallise from a solvent whilst the compounds (II), (III) and (IV) remain dissolved in the crystallising solvent to such an extent that isolated crystalline material contains only (−)-Ambrox product, or if it contains any of the other compounds (II), (III) or (IV), then they are present only in olfactory acceptable amounts.

The (−)-Ambrox may, for example, be free or substantially free of by-products (II), (III) and (IV). The selective crystallisation step may use a water miscible solvent such as ethanol or the like. The selective crystallisation of (−)-Ambrox may be influenced by the presence of unreacted homofarnesol substrate and also the ratio of (−)-Ambrox to the other detectable by-products (II), (III) and/or (IV). Even if only 10% conversion of the homofarnesol substrate to (−)-Ambrox is obtained, the selective crystallisation of (−)-Ambrox is still possible.

In the event that by-products (II) and/or (IV) may not be produced, the (−)-Ambrox may be further selectively crystallised to remove all or substantially all of by-product (III) and any unreacted homofarnesol substrate from the final (−)-Ambrox product. The term “selective crystallization” refers to a process step whereby (−)-Ambrox is caused to crystallise from a solvent whilst the compound (III) remains dissolved in the crystallising solvent to such an extent that isolated crystalline material contains only (−)-Ambrox product, or if it contains compound (III), then it is present only in olfactory acceptable amounts. The (−)-Ambrox may, for example, be free or substantially free of by-products (III). The selective crystallisation step may use a water miscible solvent such as ethanol or the like. The selective crystallisation of (−)-Ambrox may be influenced by the presence of unreacted homofarnesol substrate and also the ratio of (−)-Ambrox to the other detectable by-product (III), Even if only 10% conversion of the homofarnesol substrate to (−)-Ambrox is obtained, the selective crystallisation of (−)-Ambrox is still possible.

The olfactive purity of the final (−)-Ambrox product may be determined using a 10% ethanol extract in water or by testing the crystalline material. The final (−)-Ambrox product is tested against a commercially available reference of (−)-Ambrox product for its olfactive purity, quality and its sensory profile. The (−)-Ambrox material is also tested in application studies by experts in order to determine if the material meets the specifications with respect to its organoleptic profile.

The term “olfactively pure” as it is used in relation to (−)-Ambrox, is intended to mean that (−)-Ambrox is free of compounds (II), (III) or (IV), or any other material found in the reaction mixture, or that if such compounds or materials should be present, they are present in olfactory acceptable amounts, as that term is defined herein.

In an embodiment of the invention (−)-Ambrox in olfactively pure form contains less than 5% by weight of any of the compounds (II), (III) and/or (IV).

In an embodiment of the invention (−)-Ambrox in olfactively pure form contains less than 5% by weight of any of the compounds (II), (III), (IV) and/or any other material found in the reaction mixture.

In more particular embodiments, (−)-Ambrox in olfactively pure form contains less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or less than 0.05% by weight of each of the compounds (II), (III) and/or (IV).

In more particular embodiments, (−)-Ambrox in olfactively pure form contains less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or less than 0.05% by weight of each of the compounds (II), (III), (IV) and/or any other material found in the reaction mixture.

In more particular embodiments, (−)-Ambrox in olfactively pure form contains less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or less than 0.05% by weight but greater than 0% of each of the compounds (II), (III), (IV) and/or any other material found in the reaction mixture

The quality of separation of (−)-Ambrox from the mixture of the compounds (II), (III) and/or (IV) by selective crystallization may be influenced by the composition of the mixture from which it is separated. More particularly, the quality of the separation of (−)-Ambrox from a mixture of compounds (II), (III) and/or (IV) by crystallization was improved when the weight ratio of (−)-Ambrox to the other compounds (II), (III) and (IV) in the mixture was greater than 70:30, more particularly 80:20, more particularly 90:10, still more particularly 95:5, and more particularly still 97:3.

The quality of separation of (−)-Ambrox from the mixture of the compounds (II), (III), (IV) and/or any other material found in the reaction mixture by selective crystallization may be influenced by the composition of the mixture from which it is separated. More particularly, the quality of the separation of (−)-Ambrox from a mixture of compounds (II), (III), (IV) and/or any other material found in the reaction mixture by crystallization was improved when the weight ratio of (−)-Ambrox to the other compounds (II), (III), (IV) and any other material found in the reaction mixture in the mixture was greater than 70:30, more particularly 80:20, more particularly 90:10, still more particularly 95:5, and more particularly still 97:3.

Furthermore, the quality of the separation of (−)-Ambrox by crystallization may be influenced by the amount of unreacted homofarnesol present in the mixture from which it is separated. More particularly, the quality of separation is improved when the level of unreacted homofarnesol is less than 30% by weight, more particularly less than 20 wt %, more particularly less than 10% by weight, more particularly still less than 5 wt % and still more particularly less than 3% by weight, still more particularly less than 2% by weight, and more particularly still less than 1% by weight, based on the weight of the mixture from which (−)-Ambrox is crystallized.

Preferably, the reagents and reaction conditions employed in the bioconversion process of the present invention are such that the reaction proceeds with 100% conversion of homofarnesol, or substantially so, thus leaving no unreacted homofarnesol in the bioconversion medium. However, if unreacted homofarnesol is present, although economically disadvantageous, it can be separated from (−)-Ambrox and other by-products by distillation, or washing crystals of (−)-Ambrox with a suitable solvent, for example.

Accordingly, in a particular embodiment of the invention, there is provided a method of isolating and purifying (−)-Ambrox from a mixture comprising one or more of the compounds (II), (III) and (IV), which mixture is free, or substantially free, of homofarnesol. In a more particular embodiment, the isolation and purification of (−)-Ambrox from a mixture comprising one or more of the compounds (II), (III) and (IV), and free or substantially free of homofarnesol, is achieved by the selective crystallization of (−)-Ambrox.

(−)-Ambrox obtained according to a method of the present invention is obtained in olfacti-vely pure form. Olfactively pure (−)-Ambrox forms another aspect of the present invention.

The term “isolated” as used herein refers to a bioconversion product such as (−)-Ambrox which has been separated or purified from components which accompany it. An entity that is produced in a cellular system different from the source from which it naturally originates is “isolated”, because it will necessarily be free of components which naturally accompany it. The degree of isolation or purity can be measured by any appropriate method, e.g. gas chromatography (GC), HPLC or NMR analysis.

In some embodiments, the end product ((−)-Ambrox) is isolated and purified to homogeneity (e.g. at least 70%, 71%.72%, 73%, 74%, 75%, 76%, 77%, 78%, 79%, 80%, 81%, 82%, 83%, 84%, 85%, 86%, 87%, 88%, 89%, or 89.5% pure or 90%, 91%, 92%, 93%, 94%, 95%, 96%, 97%, 98%, 99%, or 99.5% pure).

Desirably, the amount of (−)-Ambrox produced can be from about 1 mg/l to about 20,000 mg/l (20 g/l) or higher such as from about 20 g/l to about 200 g/l or from 100-200 g/l, preferably about 125 g/l or 150 g/l or about 188 g/l or about 200 g/l or about 250 g/l or about 300 g/l or about 350 g/l or about 400 g/l or about 450 g/l

Desirably, the amount of (−)-Ambrox produced can be from about 1 mg/l to about 20,000 mg/l (20 g/l) or higher such as from about 20 g/l to about 200 g/l or from 100-200 g/l, preferably about 125 g/l or 150 g/l or about 188 g/l or about 200 g/l or about 250 g/l or about 300 g/l or about 350 g/l or about 400 g/l or about 450 g/l

At least 125 g/l (−)-Ambrox may be produced in a bioconversion reaction using a recombinant E. coli host cell producing a SHC/HAC enzyme or enzyme variant over about 2 days. It is possible to run bioconversions at 188 g/l EEH or higher (e.g. 400 g/l or 450 g/l) provided efficient mixing is achieved as stirring efficiency may be the only limitation of the system. In addition, a biocatalyst with improved activity and/or selectivity (e.g. in terms of SHC variants with further improved activity or in terms of increased SHC enzyme production and/or in terms of substrate selectivity or product selectivity) may improve or maintain productivity using less biomass or increasing substrate concentration which is advantageous with respect to mixing efficiencies.

For example about 1 to about 100 mg/l, about 30 to about 100 mg/l, about 50 to about 200 mg/l, about 100 to about 500 mg/l, about 100 to about 1,000 mg/l, about 250 to about 5,000 mg/l, about 1,000 (1 g/l) to about 15,000 mg/l (15 g/l), or about 2,000 (2 g/l) to about 10,000 mg/l (10 g/l) or about 2,000 (2 g/l) to about 25,000 mg/l (25 g/l) or about 2,000 (2 g/l) to about 25,000 mg/l (25 g/l), 26,000 mg/l (26 g/l), 27,000 mg/l (27 g/l), 28,000 mg/l (28 g/l), 29,000 mg/l (29 g/l), 30,000 mg/l (30 g/l), 40 g/l, 50 g/l, 60 g/l, 70 g/l, 80 g/l, 90 g/l, 100 g/l, 110 g/l, 120 g/l, 125 g/l, 130 g/l, 140 g/l, 150 g/l, 160 g/l, 170 g/l, 180 g/l, 190 g/l or 200 g/l or 300 g/l or 400 g/l or 450 g/l or 500 g/l of (−)-Ambrox is produced.

Preferably (−)-Ambrox at a concentration of at least 100 g/l is produced within a period of time of from 48 to 72 hours.

Preferably (−)-Ambrox at a concentration of about 150 g/l is produced within a time period of from about 48 to 72 hours. Preferably (−)-Ambrox at a concentration of about 200 g/l is produced within a time period of from about 48 to 72 hours.

Preferably (−)-Ambrox at a concentration of about 250 g/l is produced within a time period of from about 48 to 72 hours.

Preferably (−)-Ambrox at a concentration of about 300 g/l is produced within a time period of from about 48 to 72 hours.

Preferably (−)-Ambrox at a concentration of about 400 g/l is produced within a time period of from about 48 to 72 hours.

Preferably (−)-Ambrox at a concentration of about 450 g/l is produced within a time period of from about 48 to 72 hours.

The bioconversion of homofarnesol to (−)-Ambrox according to the present disclosure produces (−)-Ambrox as a predominant compound but may also produce compounds other than (−)-Ambrox which may or may not impart pleasant olfactive notes to the bioconversion mixture and so may contribute in a positive or negative manner to the sensory character of the (−)-Ambrox end product. Accordingly, a sensory analysis is carried out using well established sensory tests utilized by trained Experts (e.g. Perfumers) so that the testing can assist in determining if a chemically relevant product is also an olfactively relevant end product relative to a reference product. The removal of one of more by-product compounds from (−)-Ambrox can improve the odor of the reaction product mixture comprising (−)-Ambrox even if the removed compounds are actually odorless compounds per se. That is, an (−)-Ambrox odor enhancement may be observed in the absence of compounds 1l, Ill and IV.

Various applications for (−)-Ambrox include but are not limited to a fine fragrance or a consumer product such as fabric care, toiletries, beauty care and cleaning products, detergent products, and soap products, including essentially all products where the currently available Ambrox ingredients are used commercially, including but not limited to: Ambrox (Firmenich), Ambroxan (Henkel), Ambrofix (Givaudan), Amberlyn (Quest), Cetalox Laevo (Firmenich), Ambermor (Aromor) and Norambrenolide Ether (Pacific) products.

Thus, there is further provided herein a use of (−)-Ambrox obtained by or obtainable by a process described herein as part of a fragrance or a cosmetic or a consumer product. There is also provided herein a product comprising (−)-Ambrox obtained by or obtainable by a process described herein. The product may, for example, be a fragrance or a cosmetic or a consumer product.

### Ambra Oxide and Uses Thereof

There is further provided herein the reaction products made by the processes described herein. The reaction products may, for example, comprise, consist essentially of consist of Ambra oxide and one or more further compounds, for example one or more of a compound of formula (XI), a compound of formula (XII) and a compound of formula (XIII).

As used herein, the term “Ambra oxide” includes Ambra oxide of formula (X) below as well as Ambra oxide of formula (X) in isomerically pure form or in a mixture with one or more of the following molecules of formula (XI), (XII) and/or (XIII),

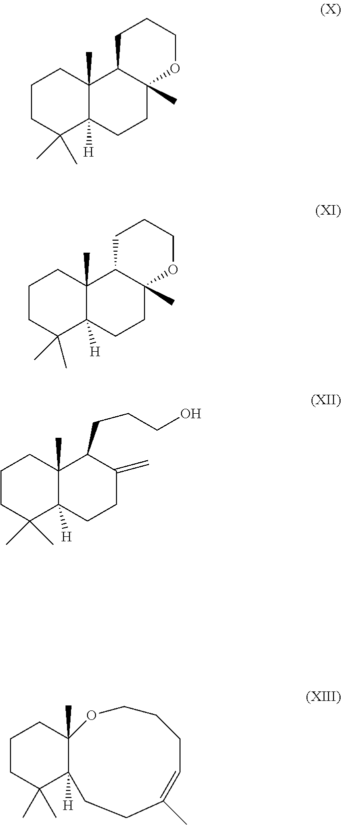 [SMILES: CC1(C)CCC[C@]2(C)[C@H]3CCCO[C@]3(C)CC[C@@H]12 ; CC1(C)CCC[C@@]2(C)[C@H]1CC[C@@]1(C)OCCC[C@@H]21 ; C=C1CC[C@H]2C(C)(C)CCC[C@]2(C)[C@H]1CCCO ; C/C1=C/CCCO[C@@]2(C)CCCC(C)(C)[C@@H]2CC1]

The nomenclature for the reaction products of formulae (X), (XI), (XII) and (XIII) is set out below.

TABLE 14
Nomenclature for the reaction products of
formulae (X), (XI), (XII) and (XIII).

| Compound | Name and Structure |
|---|---|
| (X) | 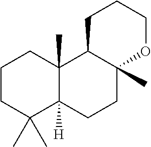 [SMILES: CC1(C)CCC[C@]2(C)[C@H]3CCCO[C@]3(C)CC[C@@H]12] |
| | (4aR,6aS,10aS,10bR)-4a,7,7,10a- |
| | tetramethyldodecahydro-1H-benzo[f]chromene] (Ambra |
| | oxide) |
| (XI) | 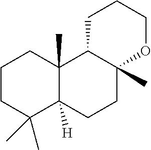 [SMILES: CC1(C)CCC[C@@]2(C)[C@H]1CC[C@@]1(C)OCCC[C@@H]21] |
| | [(4aR,6aS,10aS,10bS)-4a,7,7,10a- |
| | tetramethyldodecahydro-1H-benzo[f]chromene] |
| (XII) | 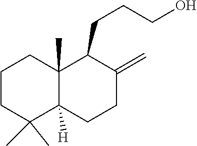 [SMILES: C=C1CC[C@H]2C(C)(C)CCC[C@]2(C)[C@H]1CCCO] |
| | [3-((1S,4aS,8aS)-5,5,8a-trimethyl-2- |
| | methylenedecahydronaphthalen-1-yl)propan-1-ol] |
| (XIII) | 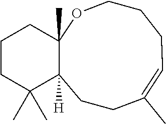 [SMILES: C/C1=C/CCCO[C@@]2(C)CCCC(C)(C)[C@@H]2CC1] |
| | [(8aS,12aS,Z)-6,9,9,12a-tetramethyl- |
| | 3,4,7,8,8a,9,10,11,12,12a-decahydro-2H-benzo[b]oxecine] |

Ambra oxide can be produced from (+)-Larixol as described in Bolster et al., Tetrahedron, 2002, 58(26), pages 5275-5285. However, it is desirable to provide alternative or improved methods for producing Ambra oxide.

The processes described herein may make Ambra oxide of formula (X) alone or in a mixture with by-products such as the compound shown in formulae (XI), (XII) and/or (XIII). For example, other stereoisomers of formula (X) may be made by the processes described herein.

There is therefore provided herein a compound of formula (X) or a composition comprising a compound of formula (X) obtained by or obtainable by the processes described herein, including all embodiments thereof.

In certain embodiments, not all of the bishomofarnesol (e.g. BisEEH) is converted to Ambra oxide or a by-product of the reaction. Therefore, the compositions described herein, for example the compositions obtained by obtainable by the processes described herein may comprise bishomofarnesol (e.g. bisEEH, for example in addition to the compound of formula (X) and/or compounds of formula (XI), (XII) and/or (XIII)). Any remaining homofarnesol may be separated from the other reaction products such that the Ambra oxide product does not comprise bishomofarnesol. In other embodiments, all of the bishomofarnesol starting material is converted to Ambra oxide of formula (X) or a by-product of the reaction by the processes described herein.

Therefore, the compositions described herein may comprise, consist essentially of or consist of one or more of a compound of formula (X), a compound of formula (XI), a compound of formula (XII), a compound of formula (XIII), bishomofarnesol starting material (e.g. bisEEH) and other stereoisomers of the compound of formula (X). For example, the compositions described herein may comprise, consist essentially of or consist of a compound of formula (X) and one or more of a compound of formula (XI), a compound of formula (XII) and a compound of formula (XIII).

For example, the compositions described herein may comprise, consist essentially of or consist of a compound of formula (X) and one or more of a compound of formula (XI), a compound of formula (XII).

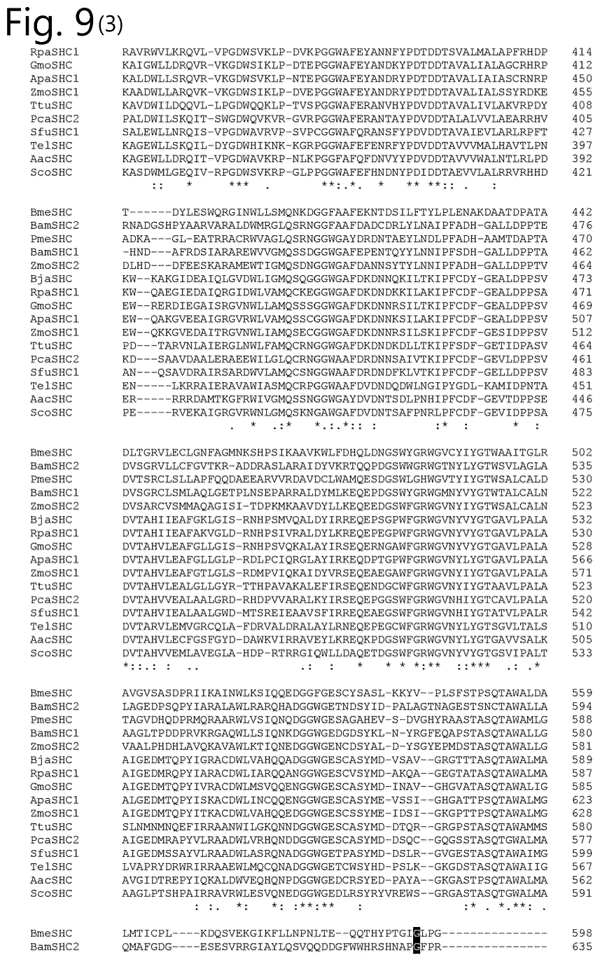

For example, the compositions described herein may comprise, consist essentially of or consist of a compound of formula (X) and a compound of formula (XII) Example 12 demonstrates that the SHC/HAC variants described herein exhibit:
an increased substrate specificity for E,E-Bis-homofarnesol (see FIG. 10), when a bis-homofarnesol substrate is used
an increased product selectivity for the E,E isomer of Bis-homofarnesol, when a bis-homofarnesol substrates are used,
an increased degree of conversion of BisEEH) as well as an increased conversion rate of BisEEH) over the first 3 to 6 hours (or over the first 4, 5, or 6 hours) of the reaction, compared to the reference SHC enzyme (e.g. wild-type SHC such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or 215G2 AacSHC or SHC #65 or a parent SHC enzyme the variant derives from such as those represented by SEQ ID NO:2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383.

Overall the observations regarding substrate specificity and product selectivity were in line with the ones described with Homofarnesol earlier herein. It was concluded that the effect of P2 mutations can be transferred to substrates other than E,E-homofarnesol, E,E-Bis-homofarnesol being one example.

The compositions described herein may therefore comprise equal to or greater than about equal to or greater than about 50 wt % of the compound of formula(X) based on the total weight of the compound of formula (X), the compound of formula (XI), the compound of formula (XII) and the compound of formula (XIII). For example, the compositions described herein may comprise equal to or greater than about 55 wt % or equal to or greater than about 60 wt % or equal to or greater than about 65 wt % or equal to or greater than about 70 wt % or equal to or greater than about 75 wt % or equal to or greater than about 80 wt % or equal to or greater than about 85 wt % or equal to or greater than about 90 wt % or equal to or greater than about 95 wt % of the compound of formula (X) based on the total weight of the compound of formula (X), the compound of formula (XI), the compound of formula (XII) and the compound of formula (XIII). The compositions described herein may, for example, comprise equal to or less than about 100 wt % of the compound of formula (X) based on the total weight of the compound of formula (X), the compound of formula (XI), the compound of formula (XII) and the compound of formula (XIII). For example, the mixture may comprise equal to or less than about 99 wt % or equal to or less than about 98 wt % or equal to or less than about 97 wt % of the compound of formula (X) based on the total weight of compound of formula (X), the compound of formula (XI), the compound of formula (XII) and the compound of formula (XIII). For example, the compositions described herein may comprise from about 50 wt % to about 100 wt % or from about 60 wt % to about 99 wt % or from about 70 wt % to about 98 wt % or from about 80 wt % to about 97 wt % or from about 90 wt % to about 97 wt % of the compound of formula (X) based on the total weight of the compound of formula (X), the compound of formula (XI), the compound of formula (XII) and the compound of formula (XIII).

The weight ratio of the compound of formula (X) to the total weight of the compound of formula (XI), the compound of formula (XII) and the compound of formula (XIII) in the compositions described herein may, for example, range from about 60:40 to about 99:1. For example, the weight ratio of the compound of formula (X) to the total weight of the compound of formula (XI), the compound of formula (XII) and the compound of formula (XIII) may range from about 65:35 to about 99:1 or from about 70:30 to about 99:1 or from about 75:25 to about 99:1 or from about 80:20 to about 99:1 or from about 85:15 to about 99:1 or from about 90:10 to about 99:1 or from about 95:5 to about 99:1. For example, the weight ratio of the compound of formula (X) to the total weight of the compound of formula (XI), the compound of formula (XII) and the compound of formula (XIII) may range from about 65:35 to about 98:2 or from about 70:30 to about 97:3 or from about 75:25 to about 96:4 or from about 80:20 to about 95:5 or from about 85:15 to about 90:10.

The weight ratio of the compound of formula (X) to bishomofarnesol (e.g. bisEEH) in the compositions described herein may, for example, range from about 90:10 to about 100:0. For example, the weight ratio of the compound of formula (X) to bishomofarnesol (e.g. bisEEH) in the compositions described herein may range from about 92:8 to about 100:0 or from about 94:6 to about 100:0 or from about 95:5 to about 100:0 or from about 96:4 to about 99.5:0.5 or from about 97:3 to about 99.0:1.0 or from about 98:2 to about 99.0:1.0.

The amount of the compound of formula (X), the compound of formula (XI), the compound of formula (XII) and the compound of formula (XIII) in a mixture of stereoisomers may, for example, be quantified by gas chromatography and/or identified by NMR spectroscopy.

Ambra oxide synthesized by the processes described herein (e.g. using SHC/HAC enzymes or variants thereof and optionally recombinant host cells) may, for example, be present in the solid phase. It may be in amorphous form or crystalline form.

The Ambra oxide produced by the methods described herein (e.g. using SHC/HAC enzymes or variants thereof and recombinant host cells) may be isolated by steam extraction/distillation or organic solvent extraction using a non-water miscible solvent (to separate the reaction products and unreacted substrate from the biocatalyst which stays in the aqueous phase) followed by subsequent evaporation of the solvent to obtain a crude reaction product as determined by gas chromatographic (GC) analysis. The steam extraction/distillation and organic solvent extraction methods are known to those skilled in the art.

By way of example, the resulting Ambra oxide may be extracted from the whole reaction mixture using an organic solvent such as a non-water miscible solvent (for example toluene). Alternatively, the resulting Ambra oxide may be extracted from the solid phase of the reaction mixture (obtained by, for example, centrifugation or filtration) using a water miscible solvent (for example ethanol) or a non-water miscible solvent (for example toluene). By way of further example, Ambra oxide is formed as solid. It may be present in the solid phase as crystals or in amorphous form and can be separated from the remaining solid phase (cell material or debris thereof) and the liquid phase also by means of filtration.

By way of further example, at a temperature above the melting point of Ambra oxide, the Ambra oxide may form an oil layer on top of aqueous phase, which oil layer can be removed and collected. In order to ensure a complete recovery of Ambra oxide after the oil layer is removed, an organic solvent may be added to the aqueous phase containing the biomass in order to extract any residual Ambra oxide contained in, or on or about the biomass. The organic layer can be combined with the oil layer, before the whole is further processed to isolate and purify Ambra oxide. The Ambra oxide may be further selectively crystallised to remove by-products (XI), (XII) and (XIII) and any unreacted bishomofarnesol substrate from the final Ambra oxide product. The term “selective crystallization” refers to a process step whereby Ambra oxide is caused to crystallise from a solvent whilst the compounds (XI), (XII) and (XIII) remain dissolved in the crystallising solvent to such an extent that isolated crystalline material contains only Ambra oxide product, or if it contains any of the other compounds (X), (XII) or (XIII), then they are present only in olfactory acceptable amounts. The Ambra oxide may, for example, be free or substantially free of by-products (XI), (XII) and (XIII). The selective crystallisation step may use a water miscible solvent such as ethanol or the like. The selective crystallisation of Ambra oxide may be influenced by the presence of unreacted homofarnesol substrate and also the ratio of Ambra oxide to the other detectable by-products (XI), (XII) and/or (XIII). Even if only 10% conversion of the homofarnesol substrate to Ambra oxide is obtained, the selective crystallisation of Ambra oxide is still possible.

The olfactive purity of the final Ambra oxide product may be determined using a 10% ethanol extract in water or by testing the crystalline material. The final Ambra oxide product is tested against a commercially available reference of Ambra oxide product for its olfactive purity, quality and its sensory profile. The Ambra oxide material is also tested in application studies by experts in order to determine if the material meets the specifications with respect to its organoleptic profile.

Examples of suitable water miscible and non-water miscible organic solvents suitable for use in the extraction and/or selective crystallization of Ambra oxide include but are not limited to aliphatic hydrocarbons, preferably those having 5 to 8 carbon atoms, such as pentane, cyclopentane, hexane, cyclohexane, heptane, octane or cyclooctane, halogenated aliphatic hydrocarbons, preferably those having one or two carbon atoms, such as dichloromethane, chloroform, carbon tetrachloride, dichloroethane or tetrachloroethane, aromatic hydrocarbons, such as benzene, toluene, the xylenes, chlorobenzene or dichlorobenzene, aliphatic acyclic and cyclic ethers or alcohols, preferably those having 4 to 8 carbon atoms, such as ethanol, isopropanol, diethyl ether, methyl tert.-butyl ether, ethyl tert.-butyl ether, dipropyl ether, diisopropyl ether, dibutyl ether, tetrahydrofuran or esters such as ethyl acetate or n-butyl acetate or ketones such as methyl isobutyl ketone or dioxane or mixtures of these. The solvents which are especially preferably used are the abovementioned heptane, Methyl tert-butyl ether (also known as MTBE, tert-butyl methyl ether, tertiary butyl methyl ether and tBME), diisopropyl ether, tetrahydrofuran, ethyl acetate and/or mixtures thereof. Preferably, a water miscible solvent such as ethanol is used for the extraction of Ambra oxide from the solid phase of the reaction mixture. The use of ethanol is advantageous because it is easy to handle, it is non-toxic and it is environmentally friendly.

The term “isolated” as used herein refers to a bioconversion product such as Ambra oxide which has been separated or purified from components which accompany it. An entity that is produced in a cellular system different from the source from which it naturally originates is “isolated”, because it will necessarily be free of components which naturally accompany it. The degree of isolation or purity can be measured by any appropriate method, e.g. gas chromatography (GC), HPLC or NMR analysis.

In some embodiments, the end product (Ambra oxide) is isolated and purified to homogeneity (e.g. at least 80%, 81%, 82%, 83%, 84%, 85%, 86%, 87%, 88%, 89%, or 89.5% pure or 90%, 91%, 92%, 93%, 94%, 95%, 96%, 97%, 98%, 99%, or 99.5% pure).

Desirably, the amount of Ambra oxide produced can be from about 1 mg/l to about 20,000 mg/l (20 g/l) or higher such as from about 20 g/l to about 200 g/l or from 100-200 g/l, preferably about 125 g/l or 150 g/l or about 188 g/l

For example about 1 to about 100 mg/l, about 30 to about 100 mg/l, about 50 to about 200 mg/l, about 100 to about 500 mg/l, about 100 to about 1,000 mg/l, about 250 to about 5,000 mg/l, about 1,000 (1 g/l) to about 15,000 mg/l (15 g/l), or about 2,000 (2 g/l) to about 10,000 mg/l (10 g/l) or about 2,000 (2 g/l) to about 25,000 mg/l (25 g/l) or about 2,000 (2 g/l) to about 25,000 mg/l (25 g/l), 26,000 mg/l (26 g/l), 27,000 mg/l (27 g/l), 28,000 mg/l (28 g/l), 29,000 mg/l (29 g/l), 30,000 mg/l (30 g/l), 40 g/l, 50 g/l, 60 g/l, 70 g/l, 80 g/l, 90 g/l, 100 g/l, 110 g/l, 120 g/l, 125 g/l, 130 g/l, 140 g/l, 150 g/l, 160 g/l, 170 g/l, 180 g/l, 190 g/l or 200 g/l or 300 g/l or 400 g/l or 500 g/l of Ambra oxide is produced. Preferably Ambra oxide at a concentration of at least 100 g/l is produced within a period of time from 48 to 72 hours.

Preferably Ambra oxide at a concentration of about 150 g/l is produced within a time period of from about 48 to 72 hours. Preferably Ambra oxide at a concentration of about 200 g/l is produced within a time period of from about 48 to 72 hours.

Preferably Ambra oxide at a concentration of about 250 g/l is produced within a time period of from about 48 to 72 hours.

The bioconversion of bishomofarnesol to Ambra oxide according to the present disclosure produces Ambra oxide as a predominant compound but may also produce compounds other than Ambra oxide which may or may not impart pleasant olfactive notes to the bioconversion mixture and so may contribute in a positive or negative manner to the sensory character of the Ambra oxide end product. Accordingly, a sensory analysis is carried out using well established sensory tests utilized by trained Experts (e.g. Perfumers) so that the testing can assist in determining if a chemically relevant product is also an olfactively relevant end product relative to a reference product. The removal of one of more by-product compounds from Ambra oxide can improve the odor of the remaining compound (Ambra oxide) even if the removed compounds are actually odorless compounds per se. That is, an Ambra oxide odor enhancement may be observed in the absence of compounds XI, XII and XIII.

Various applications for Ambra oxide include but are not limited to a fine fragrance or a consumer product such as fabric care, toiletries, beauty care and cleaning products, detergent products, and soap products, including essentially all products where the currently available Ambra oxide ingredients are used commercially.

Thus, there is further provided herein a use of Ambra oxide obtained by or obtainable by a process described herein as part of a fragrance or a cosmetic or a consumer product. There is also provided herein a product comprising Ambra oxide obtained by or obtainable by a process described herein. The product may, for example, be a fragrance or a cosmetic or a consumer product.

### Fragrance Compositions

There is further provided herein the use of the compounds and compositions described herein as or in a fragrance composition.

Thus, there is also provided herein a fragrance composition comprising one or more compounds of formula (II) or (X). A “fragrance composition” may, for example, be any composition comprising one or more compounds of formula (I) or (X) and a base material.

As used herein, the “base material” includes all known fragrance ingredients selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, diluents, and other auxiliary agents commonly used in the art.

Fragrance ingredients known to the art are readily available commercially from the major fragrance manufacturers. Non-limiting examples of such ingredients include:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-0l); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1 (2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); 3-(4-isobutyl-2-methylphenyl)propanal; maltol; methyl cedryl ketone; methylionone; verbenone; and/or vanillin;
ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicyclo[4.1.0]heptane-2,5′-[1,3]dioxane]);
macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g. isobutylquinoline (2-isobutylquinoline).

As used herein, “carrier material” means a material which is practically neutral from an odorant point of view, i.e. a material that does not significantly alter the organoleptic properties of odorants.

By “diluents” is meant any diluent conventionally used in conjunction with odorants, such as diethyl phthalate (DEP), dipropylene glycol (DPG), isopropyl myristate (IPM), triethyl citrate (TEC) and alcohol (e.g. ethanol).

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same, such as anti-oxidant adjuvant.

Said anti-oxidant may be selected, for example, from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

It might also be an ingredient that provides additional benefits such as imparting colour or texture.

It might also be an ingredient that imparts light resistance or chemical stability to one or more ingredients contained in a fragrance composition.

A detailed description of the nature and type of auxiliary agent commonly used in fragrance compositions containing same cannot be exhaustive, but it has to be mentioned that said ingredients are well known to a person skilled in the art.

There is also provided herein a consumer product comprising a compound or a composition or fragrance composition as described herein, including any embodiment thereof. The consumer product may, for example, be a cosmetic product (e.g. an eau de parfum or eau de toilette), a cleaning product, a detergent product, or a soap product.

The following numbered paragraphs define further aspects of the present disclosure.

Paragraph 1: A protein with the enzymatic activity of a Squalene-hopene-cyclase (SHC) comprising
a) a mutated amino acid sequence relative to the wild type SHC of Alicyclobacillus acidocaldarius (wild type AacSHC) of SEQ ID No. 1, wherein the W at position 169 of SEQ ID No. 1 is replaced by one amino acid selected from the group consisting of G, A and V or a functional equivalent of the mutated amino acid sequence, wherein the functional equivalent of the mutated amino acid sequence has an amino acid sequence identity from 38.0 to 99.9% to the amino acid sequence of the mutated amino acid sequence or
b) a mutated amino acid sequence relative to a wild type SHC (WT-SHC), wherein the W at a position in the amino acid sequence of the WT-SHC corresponding to W169 in the wild type AacSHC of SEQ ID No. 1 is replaced by an amino acid selected from the group consisting of G, A and V or a functional equivalent of the mutated amino acid sequence, wherein the functional equivalent of the mutated amino acid sequence has an amino acid sequence identity from 38.0 to 99.9% to the amino acid sequence of the mutated amino acid sequence.

Paragraph 2: The protein of paragraph 1, wherein the mutated amino acid sequence relative to the wild type AacSHC of SEQ ID No. 1 is the amino acid sequence of SEQ ID Nos 315, 316 or 317 Paragraph 3: The protein of paragraph 1, wherein the WT-SHC is selected from the group consisting of WT-SHC of Acetobacter pasteurianus (ApaSHC1, SEQ ID No. 24), Burkholderia ambifaria (BamSHC1, SEQ ID No. 28, BamSHC2, SEQ ID No. 29), Bradyrhizobium japonicum (BjaSHC or BjpSHC SEQ ID No. 119), Pelobacter carbinolicus or Syntrophotalea carbinolica DSM 2380 (PcaSHC2, SEQ ID No. 30), Rhodopseudomonas palustris (RpaSHC1, SEQ ID No. 31), Streptomyces coelicolor (ScoSHC, SEQ ID No. 32), Syntrophobacter fumaroxidans (SfuSHC1, SEQ ID No. 33), Teredinibacter turnerae (TtuSHC, SEQ ID No. 34), Zymomonas mobilis (ZmoSHC1, SEQ ID No. 13 or 14, ZmoSHC2, SEQ ID No. 15) and Thermosynechococcus elongatus (TeISHC, SEQ ID No. 23).

Paragraph 4: The protein of paragraph 1 or 3, wherein the mutated amino acid sequence relative to a WT-SHC of SEQ ID Nos 13, 14, 15, 23-24, 28, 29, 30, 31, 32, 33, 34 is the amino acid sequence of SEQ ID Nos 302, 303, 304.

Paragraph 5: The protein of one of the preceding paragraphs, comprising a mutated amino acid sequence, wherein a) the W at position 169 of the wild type AacSHC of SEQ ID No. 1 is replaced by G (SEQ ID No. 315, 318, 319, 320, 321,322, 333, 334, 335 336, 337, 350, 305) or b) wherein the W at position 172 in the amino acid sequence of the wild type TeISHC (SEQ ID No. 23) corresponding to W169 in the wild type AacSHC of SEQ ID No. 1 is replaced by G (SEQ ID No. 302, 304).

Paragraph 6: The protein of one of the preceding paragraphs, comprising a mutated amino acid sequence, wherein a) the G at position 600 of the wild type AacSHC of SEQ ID No. 1 is replaced by one amino acid selected from the group consisting of A, V, L, I and M (SEQ ID Nos 318, 319, 320, 321, 322, 323, 324, 325, 326, 327, 328, 329, 330, 331, 332, 305) or b) wherein the amino acid, in particular G, at a position in the amino acid sequence of the WT-SHC corresponding to G600 in the wild type AacSHC of SEQ ID No. 1 is replaced by an amino acid selected from the group consisting of A, V, L, I and M.

Paragraph 7 The protein of one of the preceding paragraphs, comprising a mutated amino acid sequence, wherein a) the A at position 306 of the wild type AacSHC of SEQ ID No. 1 is replaced by V (SEQ ID Nos 333, 334, 335, 336, 337, 338, 339, 340, 341, 342, 343, 344, 345, 346,347, 350, 351, 352) or b) wherein the amino acid, in particular P or C, at a position in the amino acid sequence of the WT-SHC corresponding to A306 in the wild type AacSHC of SEQ ID No. 1 is replaced by V.

Paragraph 8: the protein of one of the preceding paragraphs, comprising a mutated amino acid sequence, wherein a) the W at position 169 of the wild type AacSHC of SEQ ID No. 1 is replaced by G (SEQ ID No. 315, 318, 319, 320, 321,322, 333, 334, 335, 336,337, 350, 305) or b) wherein the W at a position in an amino acid sequence of the WT-SHC corresponding to W169 in the wild type AacSHC of SEQ ID No. 1 is replaced by G and the amino acid, in particular Y, Q, A or C, at the position in the amino acid sequence of the WT-SHC corresponding to S168 of the wild type AacSHC of SEQ ID No. 1 is replaced by S.

Paragraph 9: The protein of one of the preceding paragraphs comprising a mutated amino acid sequence, wherein the W at a position in an amino acid sequence of the WT-SHC corresponding to W169 in the wild type AacSHC of SEQ ID No. 1 is replaced by G and the amino acid, in particular F, at the position in the amino acid sequence of the WT-SHC corresponding to Y420 of the wild type AacSHC of SEQ ID No. 1 is replaced by Y.

Paragraph 10: The protein of one of the preceding paragraphs, comprising a mutated amino acid sequence, wherein the W at a position in an amino acid sequence of the WT-SHC corresponding to W169 in the wild type AacSHC of SEQ ID No. 1 is replaced by G the amino acid, in particular Y, Q, A or C, at the position in the amino acid sequence of the WT-SHC corresponding to S168 of the wild type AacSHC of SEQ ID No. 1 is replaced by S and the amino acid, in particular F, at the position in the amino acid sequence of the WT-SHC corresponding to Y420 of the wild type AacSHC of SEQ ID No. 1 is replaced by Y.

Paragraph 11: The protein of one of the preceding paragraphs, comprising a mutated amino acid sequence, wherein a) the W at position 169 of the wild type AacSHC of SEQ ID No. 1 is replaced by A (SEQ ID No. 316, 323, 324, 325, 326,327, 338, 339, 340, 341,342, 351) or b), wherein the W at the position in an amino acid sequence of the WT-SHC corresponding to W169 in the wild type AacSHC of SEQ ID No. 1 is replaced by A and the amino acid, in particular Y, Q, A or C, at the position in the amino acid sequence of the WT-SHC corresponding to S168 of the wild type AacSHC of SEQ ID No. 1 is replaced by S.

Paragraph 12: The protein of one of the preceding paragraphs, comprising a mutated amino acid sequence, wherein the W at a position in an amino acid sequence of the WT-SHC corresponding to W169 in the wild type AacSHC of SEQ ID No. 1 is replaced by A and the amino acid, in particular F, at the position in the amino acid sequence of the WT-SHC corresponding to Y420 of the wild type AacSHC of SEQ ID No. 1 is replaced by Y.

Paragraph 13: The protein of one of the preceding paragraphs, comprising a mutated amino acid sequence, wherein the W at a position in an amino acid sequence of the WT-SHC corresponding to W169 in the wild type AacSHC of SEQ ID No. 1 is replaced by A, the amino acid, in particular Y, Q, A or C, at the position in the amino acid sequence of the WT-SHC corresponding to S168 of the wild type AacSHC of SEQ ID No. 1 is replaced by S and the amino acid, in particular F, at the position in the amino acid sequence of the WT-SHC corresponding to Y420 of the wild type AacSHC of SEQ ID No. 1 is replaced by Y.

Paragraph 14: A nucleic acid sequence, which encodes a protein of one of the preceding claims, in particular as identified in SEQ ID No. 349.

Paragraph 15: An expression cassette, comprising the nucleic acid sequence of paragraph 14.

Paragraph 16. A vector, comprising the nucleic acid sequence of paragraph 14 or the expression cassette of claim 15.

Paragraph 17. A host cell, comprising the vector of paragraph 16 or the expression cassette of paragraph 15 or the nucleic acid sequence of paragraph 14.

Paragraph 18: A process for preparing a biocatalyst with the enzymatic activity of a SHC, comprising the following steps:
a) providing the host cell of paragraph 17, comprising a nucleic acid sequence encoding a protein according to any one of paragraphs 1 to 13,
b) cultivating the host cell of step a) in a culture medium under conditions, which allow the expression of the protein and
c) obtaining the biocatalyst, in particular the expressed protein or a host cell comprising the expressed protein.

Paragraph 19. A biocatalyst, in particular prepared according to the process of paragraph 18, wherein the biocatalyst is the protein according to any one of paragraph 1 to 13, a host cell comprising the protein according to any one of paragraph 1 to 13, a host cell lysate, freeze dried host cell, host cell extract or host cell fraction comprising the protein according to any one of paragraph 1 to 13.

Paragraph 20. A process for preparing a composition comprising (−)-Ambrox, comprising the following steps:
x) providing homofarnesol, a reaction medium and the biocatalyst of paragraph 19,
y) mixing the provided homofarnesol with the reaction medium and the biocatalyst of paragraph 19 and reacting the homofarnesol with the biocatalyst under reaction conditions so as to convert the homofarnesol to (−)-Ambrox and
z) obtaining a composition comprising (−)-Ambrox.

Paragraph 21. A process for preparing an aroma for human or animal use comprising the following steps:
i) performing a process according to paragraph 20,
j) admixing the composition comprising (−)-Ambrox obtained in step i) and at least one further component and
l) obtaining an aroma for human or animal use comprising the composition and at least one further component.

Paragraph 22. A biocatalyst comprising up to two or two or more SHC active site mutations and/or up to two or two or more site mutations other than SHC active site mutations wherein the active site mutations are selected from the active site amino acid positions (based on AacSHC) listed in Table 10 comprising active site positions 36, 168, 169, 261, 306, 307, 312, 365, 366, 420, 489, 600, 607, 609, and/or 612, preferably 169, 261, 312, 365, 489, 600, 609 and/or 612 and wherein the mutations other than active site mutations are selected from the site mutations listed in Table 7 and/or Table 8 and/or Table 9 and/or Table 18 disclosed herein, preferably comprising M132R and/or 1432T or functional equivalents, thereof wherein the biocatalyst is a protein, a host cell comprising the protein, a host cell lysate, freeze dried host cell, host cell extract or host cell fraction comprising the protein. By way of example, FIG. 19 of WO2016/170099 shows the location of the non-active site mutations identified in SHC/HAC variants 101A10, 111C8 and 215G2 (comprising M132R, 1432T and A224V) on the SHC Crystal Structure. A CAVER analysis of the AacSHC crystal structure can be performed to identify useful active site mutations and site mutations other than active site mutations useful for the preparation of a product of interest using an SHC variant comprising such beneficial active site mutations and site mutations other than active site mutations as described herein.

Paragraph 23. A process for preparing a composition comprising (−)-Ambrox or a mixture comprising (−)-Ambrox, comprising the following steps:
x) providing homofarnesol, a reaction medium and the biocatalyst of paragraph 22,
y) mixing the provided homofarnesol with the reaction medium and the biocatalyst of paragraph 22 and reacting the homofarnesol with the biocatalyst under reaction conditions so as to convert the homofarnesol to (−)-Ambrox and
z) obtaining a composition comprising (−)-Ambrox.

Paragraph 24. A process for preparing a composition comprising (−)-Ambrox or a mixture comprising (−)-Ambrox, comprising the following steps:
x) providing homofarnesol, a reaction medium and a biocatalyst disclosed in any one of Tables 2, 3, 7, 8, 9, 16 and/or 18
y) mixing the provided homofarnesol with the reaction medium and the biocatalyst disclosed in any one of Tables 2, 3, 7, 8, 9, 16 and/or 18 and reacting the homofarnesol with the biocatalyst under reaction conditions so as to convert the homofarnesol to (−)-Ambrox and
z) obtaining a composition comprising (−)-Ambrox.

Paragraph 25. A process for preparing a composition comprising Amberketal, or a mixture comprising Amberketal comprising the following steps:
x) providing hydroxyfarnesylacetone, a reaction medium and the biocatalyst disclosed in any one of Tables 2, 3, 7, 8, 9, 16 and/or 18
y) mixing the provided hydroxyfarnesylacetone with the reaction medium and the biocatalyst disclosed in any one of Tables 2, 3, 7, 8, 9, 16 and/or 18 and reacting the hydroxyfarnesylacetone with the biocatalyst under reaction conditions so as to convert the hydroxyfarnesylacetone to (−)-Amberketal and
z) obtaining a composition comprising Amberketal.

Paragraph 26. A process for preparing a composition comprising Ambra oxide or a mixture comprising Ambra oxide comprising the following steps:
x) providing (2-E)-Bishomofarnesol (BisEEH), a reaction medium and the biocatalyst disclosed in any one of Tables 2, 3, 7, 8, 9, 16 and/or 18
y) mixing the provided BisEEH with the reaction medium and the biocatalyst disclosed in any one of Tables 2, 3, 7, 8, 9, 16 and/or 18 and reacting the BisEEH with the biocatalyst under reaction conditions so as to convert the BisEEH to Ambra oxide and
z) obtaining a composition comprising Ambra oxide.

Paragraph 27: A process for preparing (−)-Ambrox or a mixture comprising (−)-Ambrox, wherein (3E,7E)-homofarnesol (EEH) or a mixture of stereoisomers comprising EEH is enzymatically converted to (−)-Ambrox or a mixture comprising (−)-Ambrox wherein the enzymatic conversion is carried out using an SHC/HAC enzyme under reaction conditions suitable for the production of (−)-Ambrox and wherein the mixture of stereoisomers comprising EEH consists essentially of homofarnesol isomers selected from one or more of the following groups consisting of [(3E,7E) and [(3Z,7E)] and/or [(3E,7E) and (3E,7Z)] and/or [(3Z,7E), (3E,7E) and (3E,7Z)] also designated as [EE:EZ], [EE:ZE] and [EE:EZ:ZE] respectively and wherein the SHC/HAC enzyme polypeptide sequence is selected from the group consisting of a SHC enzyme or SHC enzyme variant selected from Table 2, Table 3, Table 7, Table 8, Table 9, Table 16, or Table 18 and/or a sequence with a least 30%, at least 40% identity, at least 50% identity, or at least 60% identity, or at least 70% identity, or at least 80% identity, or at least 90% identity, or at least 95% identity, or at least 96% identity, or at least 97% identity, or at least 98% identity, or at least 99% identity to an SHC amino acid sequence or SHC variant amino acid sequence as selected from Table 2, Table 3, Table 7, Table 8, Table 9, Table 16, or Table 18.

Within the context of this above-mentioned aspect of the present application, SEQ ID No. 1 represents the amino acid sequence of the wild type SHC from Alicyclobacillus acidocaldarius (wild type AacSHC). SEQ ID No. 24 represents the amino acid sequence of WT-SHC of Acetobacter pasteurianus (ApaSHC). SEQ ID No. 28 represents the amino acid sequence of WT-SHC of Burkholderia ambifaria (BamSHC1). SEQ ID No. 29 represents the amino acid sequence of WT-SHC of Burkholderia ambifaria (BamSHC2) SEQ ID No. 16 represents the amino acid sequence of WT-SHC of Bradyrhizobium japonicum (BjaSHC or BjpSHC). SEQ ID No. 30 represents the amino acid sequence of WT-SHC of Pelobacter carbinolicus (PcaSHC) or Syntrophotalea carbinolica DSM 2380. SEQ ID No. 31 represents the amino acid sequence of WT-SHC of Rhodopseudomonas palustris (RpaSHC). SEQ ID No. 32 represents the amino acid sequence of WT-SHC of Streptomyces coelicolor(ScoSHC). SEQ ID No. 33 represents the amino acid sequence of WT-SHC of Syntrophobacter fumaroxidans (SfuSHC). SEQ ID No. 34 represents the amino acid sequence of WT-SHC of Teredinibacter turnerae (TtuSHC). SEQ ID No. 13 represents the amino acid sequence of WT-SHC of Zymomonas mobilis (ZmoSHC1). SEQ ID No. 15 represents the amino acid sequence of WT-SHC of Zymomonas mobilis (ZmoSHC2). SEQ ID No. 23 represents the amino acid sequence of WT-SHC of Thermosynechococcus elongatus (TeISHC, SEQ ID No. 13). SEQ ID No. 315 represents the mutated amino acid sequence of a mutant SHC from wild type AacSHC (SEQ ID No. 1), wherein W169 is exchanged by G (W169G). SEQ ID No. 316 represents the mutated amino acid sequence of a mutant SHC from wild type AacSHC (SEQ ID No. 1), wherein W169 is exchanged by A (W169A). SEQ ID No. 317 represents the mutated amino acid sequence of a mutant SHC from wild type AacSHC (SEQ ID No. 1), wherein W169 is exchanged by V (W169V). SEQ ID No. 318 represents the mutated amino acid sequence of a functional equivalent of a mutant SHC from wild type AacSHC (SEQ ID No. 1), wherein W169 is exchanged by G (W169G) and G600 is exchanged by A (G600A). SEQ ID No. 319 represents the mutated amino acid sequence of a functional equivalent of a mutant SHC from wild type AacSHC (SEQ ID No. 1), wherein W169 is exchanged by G (W169G) and G600 is exchanged by V (G600V). SEQ ID No. 320 represents the mutated amino acid sequence of a functional equivalent of a mutant SHC from wild type AacSHC (SEQ ID No. 1), wherein W169 is exchanged by G (W169G) and G600 is exchanged by L (G600L). SEQ ID No. 321 represents the mutated amino acid sequence of a functional equivalent of a functional equivalent of a mutant SHC from wild type AacSHC (SEQ ID No. 1), wherein W169 is exchanged by G (W169G) and G600 is exchanged by I (G6001). SEQ ID No. 322 represents the mutated amino acid sequence of a functional equivalent of a mutant SHC from wild type AacSHC (SEQ ID No. 1), wherein W169 is exchanged by G (W169G) and G600 is exchanged by M (G600M). SEQ ID No. 323 represents the mutated amino acid sequence of a functional equivalent of a mutant SHC from wild type AacSHC (SEQ ID No. 1), wherein W169 is exchanged by A (W169A) and G600 is exchanged by A (G600A). SEQ ID No. 324 represents the mutated amino acid sequence of a functional equivalent of a mutant SHC from wild type AacSHC (SEQ ID No. 1), wherein W169 is exchanged by A (W169A) and G600 is exchanged by V (G600V). SEQ ID No. 325 represents the mutated amino acid sequence of a functional equivalent of a mutant SHC from wild type AacSHC (SEQ ID No. 1), wherein W169 is exchanged by A (W169A) and G600 is exchanged by L (G600L). SEQ ID No. 326 represents the mutated amino acid sequence of a functional equivalent of a mutant SHC from wild type AacSHC (SEQ ID No. 1), wherein W169 is exchanged by A (W169A) and G600 is exchanged by I (G6001). SEQ ID No. 327 represents the mutated amino acid sequence of a functional equivalent of a mutant SHC from wild type AacSHC (SEQ ID No. 1), wherein W169 is exchanged by A (W169A) and G600 is exchanged by M (G600M). SEQ ID No. 328 represents the mutated amino acid sequence of a functional equivalent of a mutant SHC from wild type AacSHC (SEQ ID No. 1), wherein W169 is exchanged by V (W169V) and G600 is exchanged by A (G600A). SEQ ID No. 329 represents the mutated amino acid sequence of a functional equivalent of a mutant SHC from wild type AacSHC (SEQ ID No. 1), wherein W169 is exchanged by V (W169V) and G600 is exchanged by V (G600V). SEQ ID No. 330 represents the mutated amino acid sequence of a functional equivalent of a mutant SHC from wild type AacSHC (SEQ ID No. 1), wherein W169 is exchanged by V (W169V) and G600 is exchanged by L (G600L). SEQ ID No. 331 represents the mutated amino acid sequence of a functional equivalent of a mutant SHC from wild type AacSHC (SEQ ID No. 1), wherein W169 is exchanged by V (W169V) and G600 is exchanged by I (G6001). SEQ ID No. 332 represents the mutated amino acid sequence of a functional equivalent of a mutant SHC from wild type AacSHC (SEQ ID No. 1), wherein W169 is exchanged by V (W169V) and G600 is exchanged by M (G600M). SEQ ID No. 333 represents the mutated amino acid sequence of a functional equivalent of a mutant SHC from wild type AacSHC (SEQ ID No. 1), wherein W169 is exchanged by G (W169G), G600 is exchanged by A (G600A) and A306 is exchanged by V (A306V). SEQ ID No. 334 represents the mutated amino acid sequence of a functional equivalent of a mutant SHC from wild type AacSHC (SEQ ID No. 1), wherein W169 is exchanged by G (W169G), G600 is exchanged by V (G600V) and A306 is exchanged by V (A306V). SEQ ID No. 335 represents the mutated amino acid sequence of a functional equivalent of a mutant SHC from wild type AacSHC (SEQ ID No. 1), wherein W169 is exchanged by G (W169G), G600 is exchanged by L (G600L) and A306 is exchanged by V (A306V). SEQ ID No. 336 represents the mutated amino acid sequence of a functional equivalent of a mutant SHC from wild type AacSHC (SEQ ID No. 1), wherein W169 is exchanged by G (W169G), G600 is exchanged by I (G6001) and A306 is exchanged by V (A306V). SEQ ID No. 337 represents the mutated amino acid sequence of a functional equivalent of a mutant SHC from wild type AacSHC (SEQ ID No. 1), wherein W169 is exchanged by G (W169G), G600 is exchanged by M (G600M) and A306 is exchanged by V (A306V). SEQ ID No. 338 represents the mutated amino acid sequence of a functional equivalent of a mutant SHC from wild type AacSHC (SEQ ID No. 1), wherein W169 is exchanged by A (W169A), G600 is exchanged by A (G600A) and A306 is exchanged by V (A306V). SEQ ID No. 339 represents the mutated amino acid sequence of a functional equivalent of a mutant SHC from wild type AacSHC (SEQ ID No. 1), wherein W169 is exchanged by A (W169A), G600 is exchanged by V (G600V) and A306 is exchanged by V (A306V). SEQ ID No. 340 represents the mutated amino acid sequence of a functional equivalent of a mutant SHC from wild type AacSHC (SEQ ID No. 1), wherein W169 is exchanged by A (W169A), G600 is exchanged by L (G600L) and A306 is exchanged by V (A306V). SEQ ID No. 341 represents the mutated amino acid sequence of a functional equivalent of a mutant SHC from wild type AacSHC (SEQ ID No. 1), wherein W169 is exchanged by A (W169A), G600 is exchanged by I (G6001) and A306 is exchanged by V (A306V). SEQ ID No. 342 represents the mutated amino acid sequence of a functional equivalent of a mutant SHC from wild type AacSHC (SEQ ID No. 1), wherein W169 is exchanged by A (W169A), G600 is exchanged by M (G600M) and A306 is exchanged by V (A306V). SEQ ID No. 343 represents the mutated amino acid sequence of a functional equivalent of a mutant SHC from wild type AacSHC (SEQ ID No. 1), wherein W169 is exchanged by V (W169V), G600 is exchanged by A (G600A) and A306 is exchanged by V (A306V). SEQ ID No. 344 represents the mutated amino acid sequence of a functional equivalent of a mutant SHC from wild type AacSHC (SEQ ID No. 1), wherein W169 is exchanged by V (W169V), G600 is exchanged by V (G600V) and A306 is exchanged by V (A306V). SEQ ID No. 345 represents the mutated amino acid sequence of a functional equivalent of a mutant SHC from wild type AacSHC (SEQ ID No. 1), wherein W169 is exchanged by V (W169V), G600 is exchanged by L (G600L) and A306 is exchanged by V (A306V). SEQ ID No. 346 represents the mutated amino acid sequence of a functional equivalent of a mutant SHC from wild type AacSHC (SEQ ID No. 1), wherein W169 is exchanged by V (W169V), G600 is exchanged by I (G6001) and A306 is exchanged by V (A306V). SEQ ID No. 347 represents the mutated amino acid sequence of a functional equivalent of a mutant SHC from wild type AacSHC (SEQ ID No. 1), wherein W169 is exchanged by V (W169V), G600 is exchanged by M (G600M) and A306 is exchanged by V (A306V). SEQ ID No. 348 represents the mutated amino acid sequence of the benchmark SHC from wild type AacSHC (SEQ ID No. 1), wherein M132 is exchanged by R (M132R), A224 is exchanged by V (A224V) and 1432 is exchanged by T (1432T). SEQ ID No. 349 represents the nucleotide sequence of the wild type SHC from Alicyclobacillus acidocaldarius (wild type AacSHC, SEQ ID No. 1). SEQ ID No. 350 represents the mutated amino acid sequence of a functional equivalent of a mutant SHC from wild type AacSHC (SEQ ID No. 1), wherein W169 is exchanged by G (W169G) and A306 is exchanged by V (A306V). SEQ ID No. 351 represents the mutated amino acid sequence of a functional equivalent of a mutant SHC from wild type AacSHC (SEQ ID No. 1), wherein W169 is exchanged by A (W169A) and A306 is exchanged by V (A306V).

SEQ ID No. 352 represents the mutated amino acid sequence of a functional equivalent of a mutant SHC from wild type AacSHC (SEQ ID No. 1), wherein W169 is exchanged by V (W169V) and A306 is exchanged by V (A306V). SEQ ID No. 302 represents the mutated amino acid sequence of a functional equivalent of a mutant SHC from wild type TeISHC (SEQ ID No. 23), wherein W172 is exchanged by G (W172G), P311 is exchanged by A (P311A), F425 is exchanged by Y (F425Y) and G609 is exchanged by A (G609A). SEQ ID No. 303 represents the mutated amino acid sequence of a functional equivalent of a mutant SHC from wild type ZmoSHC1 (SEQ ID No. 13), wherein Q221 is exchanged by S (Q221S), W222 is exchanged by G (W222G), A368 is exchanged by V (A368V), F486 is exchanged by Y (F486Y) and G667 is exchanged by A (G667A). SEQ ID No. 304 represents the mutated amino acid sequence of a mutant SHC from wild type TeISHC (SEQ ID No. 23), wherein W172 is exchanged by G (W172G). SEQ ID No. 305 represents the mutated amino acid sequence of a functional equivalent of a mutant SHC from wild type AacSHC (SEQ ID No. 1), wherein W169 is exchanged by G (W169G), G600 is exchanged by M (G600M), M132 is exchanged by R (M132R), A224 is exchanged by V (A224V) and 1432 is exchanged by T (1432T).

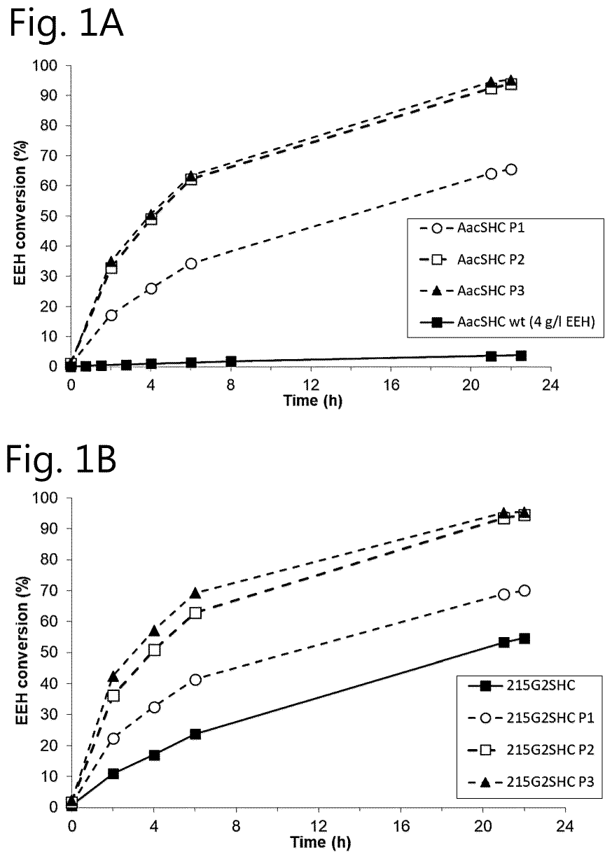

FIG. 19 of WO2016/170099 and FIG. 1 of Eichhorn et al Adv.Synth.Catal (2018) 360:2339-2351 show the location of the site mutations other than active site mutations identified in SHC/HAC variants 101A10, 111C8 and 215G2 (comprising M132R, 1432T and A224V) on the SHC Crystal Structure (in colour): red for variant 215G2; purple (wine red) for variant 101A10 and green for variant 111C8. For the amino acids identified at as being responsible for the increased activity, the side-chains are highlighted in yellow in the co-crystallized substrate analog. Other mutations for identified variants with no improved activity are marked in blue. It is noted that blue mutations are spread about half-half (i.e. 50:50) over the 2 domains of the enzyme, whereas the beneficial AacSHC mutations which were identified are located mostly (apart from one) in domain 2. The only exception is the mutation F601Y which is in the vicinity of the active site. FIG. 20 WO2016/170099 and FIG. 1 of Eichhorn eta/Adv.Synth.Catal (2018) 360:2339-2351 show the following mutations (in black and white): mutations having no beneficial effect on SHC/HAC activity are shown in black, they are spread over the 2 domains of the SHC enzyme. In grey are shown the mutations identified in SHC variants (101A10, 111C8 and 215G2) showing improved SHC/HAC activity, they are located with only one exception in domain 2 of the SHC enzyme. The side chain of the mutations contributing to the improved activity of the variants are highlighted, This improvement disclosed herein can be attributed to an increase in total turnover number (total number of reactions that one enzyme molecule can catalyse) when particular mutations are introduced in WT or SHC variants. This increased total turnover number may be the result of an increased catalytic potential or an increased stability of the resulting SHC enzyme variant, or a combination of the two. An increased stability may be the result of the optimal reaction conditions for the SHC variant with, for example, significantly lower temperature and SDS concentration.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. The term “comprising” also means “including” as well as “consisting” e.g. a composition “comprising” X may consist exclusively of X or may include something additional e.g. X+Y. It must be noted also that, as used in this specification and the appended claims, the singular forms “a”, “an” and “the” include plural referents unless the content clearly dictates otherwise. By way of example, a reference to “a gene” or “an enzyme” is a reference to “one or more genes” or “one or more enzymes”.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristics of the invention. In addition, the verb “to consist” may be replaced by “to consist essentially of” meaning that a method or use as described herein may comprise additional step(s) than the ones specifically identified, said additional step(s) not altering the unique characteristic of the invention. In addition, the verb “to consist” may be replaced by “to consist essentially of” meaning that a nucleotide or amino acid sequence as described herein may comprise additional nucleotides or amino acids than the ones specifically identified, said additional nucleotides or amino acids not altering the unique characteristics of the invention.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 1% of the value.

As used herein, the term “and/or” is understood to mean that all members of a group connected by the term “and/or” are represented both cumulatively with respect to each other in any combination, and alternatively with respect to each other. Exemplarily, for the expression “A, B and/or C”, the following disclosure is to be understood thereunder: i) (A or B or C), or ii) (A and B), or iii) (A and C), or iv) (B and C), or v) (A and B and C), or vi) (A and B or C), or vii) (A or B and C), or viii) (A and C or B).

As used herein, the term “substantially free of” is understood to mean that a compound or composition is largely free of other components but is not guaranteed to be 100% pure as trace amounts of other not deliberately added components may be present. The term allows for the presence of other components provided that they do not affect the basic characteristics of a claimed compound or composition.

Various embodiments are described herein. Each embodiment as identified herein may be combined together unless otherwise indicated. It is to be understood that this disclosure is not limited to the particular methodology, protocols and reagents described herein as these may vary.

It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by the person skilled in the art. In accordance with the present disclosure there may be conventional molecular biology, microbiology, and recombinant DNA techniques employed which are within the skill of the art.

This disclosure is not limited in its application to the details of construction and the arrangement of components set forth in the following description or illustrated in the drawings. The disclosure is capable of other embodiments and of being practiced or of being carried out in various ways.

Also, the phraseology and terminology used herein is for the purpose of description and should not be regarded as limiting. Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety.

Stocks of materials for use in bioconversion reactions (125 g/l substrate or higher) were prepared either in g/l (for example a biocatalyst suspension) or w/w % (for example SDS) concentrations. Bioconversion reactions were set up by adding by weight the required amounts of individual components (for example stocks of substrate, biocatalyst suspension, SDS solution, reaction buffer) as described in, for example Example 4. For this reason, the actual volume of the reaction mixture is expressed in mass units and not in volume units (see for example Example 4). Because the substrate used is an oil (density <1), a total reaction mass of e.g. 150 g (as for example in Example 4) does not translate into a reaction volume of 150 ml. Nonetheless the concentrations of the individual components in the bioconversion reaction mixtures are indicated in mass per volume (for example g/l or (w/v) %) and thus do not take into account that a total reaction mass of e.g. 150 g (as for example in. Example 4) does not correspond exactly to a reaction volume of 150 ml (as for example in Example 4).

For the avoidance of doubt, all reference to EEH in the Examples, the Figures and the legends to the Figures is a reference to the EE isomer of homofarnesol. The EEH used is taken from a homofarnesol stock comprising an EE:EZ isomer mixture in a weight ratio of 80:20.

The examples described herein are illustrative of the present disclosure and are not intended to be limitations thereon. Different embodiments of the present disclosure have been described according to the present disclosure. Many modifications and variations may be made to the techniques described and illustrated herein without departing from the spirit and scope of the disclosure. Accordingly, it should be understood that the examples are illustrative only and are not limiting upon the scope of the disclosure.

The following numbered paragraphs define further aspects of the present disclosure.

Paragraph 28: A process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant, wherein the SHC/HAC enzyme variant has an amino acid sequence having at least 30.0%, Identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 and wherein the SHC/HAC enzyme variant amino acid sequence has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 at positions corresponding to positions W169, A306 and/or G600 of SEQ ID NO: 1, 2, or 3 or at a position in an amino acid sequence of a wild type SHC/HAC enzyme corresponding to W169, A306 and/or G600 of SEQ ID NO: 1, 2, or 3 wherein the W at position 169 or corresponding to position 169 of SEQ ID NO: 1, 2 or 3 is replaced by G; wherein the A at position 306 or corresponding to position 306 of SEQ ID NO: 1, 2 or 3 is replaced by V; and/or wherein the G at position 600 or corresponding to 600 of SEQ ID NO: 1, 2 or 3 is replaced by M.

Sequence identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% or 100% identity or similarity to the polypeptide identified.

Paragraph 29: A process for preparing Ambra oxide or a mixture comprising Ambra oxide, the process comprising enzymatically converting (2,E)-Bishomofarnesol (BisEEH) or a mixture of isomers of bishomofarnesol comprising BisEEH to Ambra oxide or a mixture comprising Ambra oxide using a SHC/HAC enzyme variant, wherein the SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 and wherein the SHC/HAC enzyme variant amino acid sequence has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290. 291, 292, 293, 294, 295, 296, 307, 308, 309 or 310 at positions corresponding to positions W169, A306 and/or G600 of SEQ ID NO: 1, 2, or 3 or at a position in an amino acid sequence of a wild type SHC/HAC enzyme corresponding to W169, A306 and/or G600 of SEQ ID NO:1, 2, or 3 wherein the W at position 169 or corresponding to position 169 of SEQ ID NO: 1, 2 or 3 is replaced by G; wherein the A at position 306 or corresponding to position 306 of SEQ ID NO: 1, 2 or 3 is replaced by V; and/or wherein the G at position 600 or corresponding to position 600 of SEQ ID NO: 1, 2 or 3 is replaced by M.

Sequence identity or similarity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% or 100% identity or similarity to the polypeptide identified.

Paragraph 30:A process according to paragraph 28 or 29, wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, A or V or a functional equivalent thereof, preferably wherein the W at position 169 is replaced by G, and/or
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by A, V, L, I or M or a functional equivalent thereof.

In an embodiment, the process according to paragraph 30, is such that:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G or a functional equivalent thereof, and/or
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by A, V, L, I or M or a functional equivalent thereof.

In an embodiment, the process according to paragraph 30 is such that:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, A or V or a functional equivalent thereof, preferably wherein the W at position 169 is replaced by G, and/or
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by V, L, I or M or a functional equivalent thereof.

In an embodiment, the process according to paragraph 30 is such that:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G or a functional equivalent thereof and/or
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by V, L, I or M or a functional equivalent thereof.

Paragraph 31:A process according to any one of the preceding paragraphs, wherein the amino acid at position 168 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290. 291, 292, 293, 294, 295, 296, 307, 308, 309 or 310 is S or the amino acid at a position in an amino acid sequence of a wild type SHC corresponding to 168 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290. 291, 292, 293, 294, 295, 296, 307, 308, 309 or 310 is S.

Paragraph 32: A process according to any one of the preceding paragraphs, wherein
the SHC/HAC enzyme variant has an amino acid sequence having at least 70.0% identity to SEQ ID NO: 1, 4, 5, 6 or 350 and has at least one of the following mutations: W169G, A306V and G600M or
the SHC/HAC enzyme variant has an amino acid sequence having at least 70.0% identity to SEQ ID NO: 7, 8, 9 or 386 and has the following mutations: M132R, A224V, 1432T, A557T, R613S, and has at least one of the following mutations: W169G, A306V and G600Mor
the SHC/HAC enzyme variant has an amino acid sequence having at least 70.0% identity to SEQ ID NO: 10, 11, 12 or 385 and has the following mutations: M132R, A224V, 1432T and has at least one of the following mutations: W169G, A306V and G600M.

Sequence identity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the polypeptide identified.

Paragraph 33: A process according to any one of the preceding paragraphs, wherein
the SHC/HAC enzyme variant has an amino acid sequence having at least 70.0% identity to SEQ ID NO: 23, 304, 359 or 360 and has at least one of the following mutations: W172G, A311V and G609M (Tel SHC/HAC variant) or
the SHC/HAC enzyme variant has an amino acid sequence having at least 70.0% identity to SEQ ID NO: 32, 311, 357, or 358 and has the following mutations: W196G, A335V and G629M (Sco SHC/HAC variant) or
the SHC/HAC enzyme variant has an amino acid sequence having at least 70.0% identity to SEQ ID NO:13, 312, 353 or 354 and has at least one of the following mutations: W222G, A368V and G667M (Zmo SHC1 variant) or
the SHC/HAC enzyme variant has an amino acid sequence having at least 70.0% identity to SEQ ID NO: 15, 313, 355 or 356 and has at least one of the following mutations: W177G, A321V and G619M (Zmo SHC2 variant).

Sequence identity may be of at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the polypeptide identified.

Paragraph 34: A process according to any one of the preceding paragraphs, wherein the SHC/AHC enzyme variant has an amino acid sequence comprising or consisting of or essentially consisting of SEQ ID NO: 4, 5, 6, 7, 8, 9, 10, 11, 12, 305, 306, 304, 302, 311, 312, 313, 353, 354, 355, 356, 357, 358, 359, 360, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 350, 351 or 352.

Paragraph 35: A process according to any one of the preceding paragraphs, wherein, the SHC/HAC enzyme variant disclosed herein exhibits:
an increased substrate specificity for EEH (or for BisEEH) when homofarnesol or bis-homofarnesol substrates are used,
an increased product selectivity for (−)-Ambrox (or Ambra oxide) when homofarnesol or bis-homofarnesol substrates are used,
an increased specificity for a particular isomer of substrates when substrates other than homofarnesol or bis-homofarnesol are used (eg ethyl-homofarnesol, hydroxyfarnesylacetone and the like—see, for example WO2021/110858 and WO2021/209482 respectively)
an increased productivity and/or
an increased degree of conversion of EEH (or BisEEH) as well as an increased conversion rate of EEH (or BisEEH) over the first 3 to 6 hours (or over the first 4, 5, or 6 hours) of the reaction, compared to the reference SHC enzyme (e.g. wild-type SHC such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290. 291, 292, 293, 294, 295, 296, 307, 308, 309, 310 or 215G2 AacSHC or SHC #65 or a parent SHC enzyme the variant derives from such as those represented by SEQ ID NO:2, 3, 47-48, 49, 50, 51, 52, 53, 54, 361, 362, 363, 364, 365, 366, 367, 368, 369, 370, 371, 372, 373, 374, 375, 376, 377, 378, 379, 380, 381, 382 or 383.

Paragraph 36: A SHC/HAC enzyme variant as defined in any one of the preceding paragraphs, preferably wherein the variant comprises: a G at position 169 or corresponding to position 169 of SEQ ID NO: 1, 2 or 3;
a G at position 169 or corresponding to position 169 of SEQ ID NO: 1, 2 or 3 and a V at position 306 or corresponding to position 306 of SEQ ID NO: 1, 2 or 3; or
a G at position 169 or corresponding to position 169 of SEQ ID NO: 1, 2 or 3 and an M at position 600 or corresponding to position 306 of SEQ ID NO: 1, 2 or 3; or
a G at position 169 or corresponding to position 169 of SEQ ID NO: 1, 2 or 3 and a V at position 306 or corresponding to position 306 of SEQ ID NO: 1, 2 or 3 and an M at position 600 or corresponding to position 600 of SEQ ID NO: 1, 2 or 3,
And/or wherein the G at position 600 or corresponding to 600 of SEQ ID NO: 1, 2 or 3 is replaced by M.

Paragraph 37: A process according to any one of the preceding paragraphs except paragraph 36, wherein the enzymatic conversion takes place at a temperature in the range of about 30° C. to about 50° C., for example from about 40° to about 50° C., and/or at a pH in the range of about 5 to about 6.

Paragraph 38: A process according to any one of the preceding paragraphs except paragraph 36, wherein the process comprises culturing a recombinant host cell that produces the SHC/HAC enzyme variant.

Paragraph 39: A process according to paragraph 38, wherein the recombinant host cells comprise a nucleic acid sequence selected from SEQ ID NO: 38, 39, 40, 41, 42, 43, 44, 45 and/or 46.

Paragraph 40:A process according to any one of the preceding paragraphs except paragraph 36, wherein the mixture of isomers of homofarnesol comprising EEH is an EE:EZ isomer mixture, preferably wherein the EE:EZ isomer mixture is in a weight ratio of 80:20.

Paragraph 41: A process according to any one of the preceding paragraphs except paragraph 36, wherein the weight ratio of the recombinant host cell producing the SHC/HAC enzyme variant to EEH or a mixture of isomers of homofarnesol comprising EEH in an EE:EZ isomer mixture in a weight ratio of 80:20 is about 2:1, 1:1 or about 0.5:1 or about 0.1:1.

Paragraph 42: (−)-Ambrox obtained by or obtainable by the process of any preceding paragraphs except paragraph 36, in a solid form in an amorphous or crystalline form.

Paragraph 43: Use of (−)-Ambrox of claim 15 as part of a fragrance or a cosmetic or a consumer product such as fabric care, toiletry, beauty care, a cleaning product, a detergent product, and/or a soap product.

Paragraph 44: A fragrance or a cosmetic or a consumer product comprising (−)-Ambrox of paragraph 42.

Paragraph 45: A nucleic acid sequence encoding the SHC/HAC enzyme variant of paragraph 36.

Paragraph 46: A construct comprising the nucleic acid sequence of paragraph 45.

Paragraph 47: A recombinant host cell comprising the nucleic acid sequence of paragraph 45 or the construct of paragraph 46.

Paragraph 48: A process for preparing (−)-Ambrox or a mixture comprising (−)-Ambrox according to any one of paragraphs 28, 29 or 31, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant, wherein the SHC/HAC enzyme variant has an amino acid sequence having at least 30.0%, identity or similarity to SEQ ID NO: 1-12, 16, 17, 18, 19, 20, 21, 22, 23, 24, 25, 26, 27, 28, 29, 30, 31, 32, 33, 34, 47-54, 55-296, 301-313, 315-353, 354-383 wherein the W at position 169 or corresponding to position 169 of SEQ ID NO: 1, is replaced by G; wherein the A at position 306 or corresponding to position 306 of SEQ ID NO: 1, 2 or 3 is replaced by V; and/or wherein the G at position 600 or corresponding to 600 of SEQ ID NO: 1, 2 or 3 is replaced by M.

Sequence identity or similarity may be at least 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The process according to any one of the preceding paragraphs, wherein the process is carried out on an industrial scale, preferably where the bioconversion volume is at least about 100L, more preferably in the range of about 1000L to about 3000L, even more preferably about 10,000L, even more preferably about 100,000L, or even about 250,000L.

The following numbered paragraphs define further aspects of the present disclosure.

Paragraph 49: A protein with the enzymatic activity of a Squalene-hopene-cyclase (SHC) comprising
a) a mutated amino acid sequence relative to the wild type SHC of Alicyclobacillus acidocaldarius (wild type AacSHC) of SEQ ID No. 1, wherein the W at position 169 of SEQ ID No. 1 is replaced by amino acid G, or a functional equivalent of the mutated amino acid sequence, wherein the functional equivalent of the mutated amino acid sequence has an amino acid sequence identity from 38.0 to 99.9% to the amino acid sequence of the mutated amino acid sequence or
b) a mutated amino acid sequence relative to a wild type SHC (WT-SHC), wherein the W at a position in the amino acid sequence of the WT-SHC corresponding to W169 in the wild type AacSHC of SEQ ID No. 1 is replaced by amino acid G, or a functional equivalent of the mutated amino acid sequence, wherein the functional equivalent of the mutated amino acid sequence has an amino acid sequence identity from 38.0 to 99.9% to the amino acid sequence of the mutated amino acid sequence.

Sequence identity may be of at least 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%,70%, 71%, 72%, 73%, 74%, 75%, 76%, 77%, 78%, 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, 99.9% or 100% identity to the polypeptide identified.

Paragraph 50: The protein of paragraph 49, wherein the mutated amino acid sequence relative to the wild type AacSHC of SEQ ID No. 1 is the amino acid sequence of SEQ ID Nos 315.

Paragraph 51: The protein of paragraph 49, wherein the WT-SHC is selected from the group consisting of WT-SHC of Acetobacter pasteurianus (ApaSHC1, SEQ ID No. 24), Burkholderia ambifaria (BamSHC1, SEQ ID No. 28, BamSHC2, SEQ ID No. 29), Bradyrhizobium japonicum (BjaSHC or BjpSHC SEQ ID No. 119), Pelobacter carbinolicus or Syntrophotalea carbinolica DSM 2380 (PcaSHC2, SEQ ID No. 30), Rhodopseudomonas palustris (RpaSHC1, SEQ ID No. 31), Streptomyces coelicolor (ScoSHC, SEQ ID No. 32), Syntrophobacter fumaroxidans (SfuSHC1, SEQ ID No. 33), Teredinibacter turnerae (TtuSHC, SEQ ID No. 34), Zymomonas mobilis (ZmoSHC1, SEQ ID No. 13 or 14, ZmoSHC2, SEQ ID No. 15) and Thermosynechococcus elongatus (TeISHC, SEQ ID No. 23).

Paragraph 52: The protein of paragraph 49 or 51, wherein the mutated amino acid sequence relative to a WT-SHC of SEQ ID Nos 13, 14, 15, 23-24, 28, 29, 30, 31, 32, 33, 34 is the amino acid sequence of SEQ ID Nos 302, 303, 304.

Paragraph 53: The protein of one of the preceding paragraphs, comprising a mutated amino acid sequence, wherein a) the W at position 169 of the wild type AacSHC of SEQ ID No. 1 is replaced by G (SEQ ID No. 315, 318, 319, 320, 321,322, 333, 334, 335 336, 337, 350, 305) or b) wherein the W at position 172 in the amino acid sequence of the wild type TeISHC (SEQ ID No. 23) corresponding to W169 in the wild type AacSHC of SEQ ID No. 1 is replaced by G (SEQ ID No. 302, 304).

Paragraph 54: The protein of one of the preceding paragraphs, comprising a mutated amino acid sequence, wherein a) the G at position 600 of the wild type AacSHC of SEQ ID No. 1 is replaced by amino acid selected from the group consisting of A, V, L, I and M (SEQ ID Nos 318, 319, 320, 321, 322, 323, 324, 325, 326, 327, 328, 329, 330, 331, 332, 305) or b) wherein the amino acid, in particular G, at a position in the amino acid sequence of the WT-SHC corresponding to G600 in the wild type AacSHC of SEQ ID No. 1 is replaced by amino acid selected from the group consisting of A, V, L, I and M.

Paragraph 55 The protein of one of the preceding paragraphs, comprising a mutated amino acid sequence, wherein a) the A at position 306 of the wild type AacSHC of SEQ ID No. 1 is replaced by V (SEQ ID Nos 333, 334, 335, 336, 337, 338, 339, 340, 341, 342, 343, 344, 345, 346,347, 350, 351, 352) or b) wherein the amino acid, in particular P or C, at a position in the amino acid sequence of the WT-SHC corresponding to A306 in the wild type AacSHC of SEQ ID No. 1 is replaced by V.

Paragraph 56: the protein of one of the preceding paragraphs, comprising a mutated amino acid sequence, wherein a) the W at position 169 of the wild type AacSHC of SEQ ID No. 1 is replaced by G (SEQ ID No. 315, 318, 319, 320, 321,322, 333, 334, 335, 336,337, 350, 305) or b) wherein the W at a position in an amino acid sequence of the WT-SHC corresponding to W169 in the wild type AacSHC of SEQ ID No. 1 is replaced by G and the amino acid, in particular Y, Q, A or C, at the position in the amino acid sequence of the WT-SHC corresponding to S168 of the wild type AacSHC of SEQ ID No. 1 is replaced by S.

Paragraph 57: The protein of one of the preceding paragraphs comprising a mutated amino acid sequence, wherein the W at a position in an amino acid sequence of the WT-SHC corresponding to W169 in the wild type AacSHC of SEQ ID No. 1 is replaced by G and the amino acid, in particular F, at the position in the amino acid sequence of the WT-SHC corresponding to Y420 of the wild type AacSHC of SEQ ID No. 1 is replaced by Y.

Paragraph 58: The protein of one of the preceding paragraphs, comprising a mutated amino acid sequence, wherein the W at a position in an amino acid sequence of the WT-SHC corresponding to W169 in the wild type AacSHC of SEQ ID No. 1 is replaced by G the amino acid, in particular Y, Q, A or C, at the position in the amino acid sequence of the WT-SHC corresponding to S168 of the wild type AacSHC of SEQ ID No. 1 is replaced by S and the amino acid, in particular F, at the position in the amino acid sequence of the WT-SHC corresponding to Y420 of the wild type AacSHC of SEQ ID No. 1 is replaced by Y.

Paragraph 59: The protein of one of the preceding paragraphs, comprising a mutated amino acid sequence, wherein a) the W at position 169 of the wild type AacSHC of SEQ ID No. 1 is replaced by G (SEQ ID No. 315, 318, 319, 320, 321,322, 333, 334, 335 336, 337, 350, 305) (or b), wherein the W at the position in an amino acid sequence of the WT-SHC corresponding to W169 in the wild type AacSHC of SEQ ID No. 1 is replaced by G and the amino acid, in particular Y, Q, A or C, at the position in the amino acid sequence of the WT-SHC corresponding to S168 of the wild type AacSHC of SEQ ID No. 1 is replaced by S.

Paragraph 60: The protein of one of the preceding paragraphs, comprising a mutated amino acid sequence, wherein the W at a position in an amino acid sequence of the WT-SHC corresponding to W169 in the wild type AacSHC of SEQ ID No. 1 is replaced by G (SEQ ID No. 315, 318, 319, 320, 321,322, 333, 334, 335 336, 337, 350, 305) and the amino acid, in particular F, at the position in the amino acid sequence of the WT-SHC corresponding to Y420 of the wild type AacSHC of SEQ ID No. 1 is replaced by Y.

Paragraph 61: The protein of one of the preceding paragraphs, comprising a mutated amino acid sequence, wherein the W at a position in an amino acid sequence of the WT-SHC corresponding to W169 in the wild type AacSHC of SEQ ID No. 1 is replaced by G (SEQ ID No. 315, 318, 319, 320, 321,322, 333, 334, 335 336, 337, 350, 305) amino acid, in particular Y, Q, A or C, at the position in the amino acid sequence of the WT-SHC corresponding to S168 of the wild type AacSHC of SEQ ID No. 1 is replaced by S and the amino acid, in particular F, at the position in the amino acid sequence of the WT-SHC corresponding to Y420 of the wild type AacSHC of SEQ ID No. 1 is replaced by Y.

Paragraph 62: A nucleic acid sequence, which encodes a protein of one of the preceding paragraphs s, in particular as identified in SEQ ID No. 349.

Paragraph 63: An expression cassette, comprising the nucleic acid sequence of paragraph 62.

Paragraph 64. A vector, comprising the nucleic acid sequence of paragraph 62 or the expression cassette of claim 63.

Paragraph 65. A host cell, comprising the vector of paragraph 64 or the expression cassette of paragraph 63 or the nucleic acid sequence of paragraph 62.

Paragraph 66: A process for preparing a biocatalyst with the enzymatic activity of a SHC, comprising the following steps:
a) providing the host cell of paragraph 65, comprising a nucleic acid sequence encoding a protein according to any one of paragraphs 49 to 61,
b) cultivating the host cell of step a) in a culture medium under conditions, which allow the expression of the protein and
c) obtaining the biocatalyst, in particular the expressed protein or a host cell comprising the expressed protein.

Paragraph 67. A biocatalyst, in particular prepared according to the process of paragraph 66, wherein the biocatalyst is the protein according to any one of paragraph 49 to 61, a host cell comprising the protein according to any one of paragraph 49 to 61, a host cell lysate, freeze dried host cell, host cell extract or host cell fraction comprising the protein according to any one of paragraph 49 to 61.

Paragraph 68. A process for preparing a composition comprising (−)-Ambrox, comprising the following steps:
x) providing homofarnesol, a reaction medium and the biocatalyst of paragraph 67,
y) mixing the provided homofarnesol with the reaction medium and the biocatalyst of paragraph 19 and reacting the homofarnesol with the biocatalyst under reaction conditions so as to convert the homofarnesol to (−)-Ambrox and
z) obtaining a composition comprising (−)-Ambrox.

Paragraph 69. A process for preparing an aroma for human or animal use comprising the following steps:
i) performing a process according to paragraph 68,
j) admixing the composition comprising (−)-Ambrox obtained in step i) and at least one further component and
l) obtaining an aroma for human or animal use comprising the composition and at least one further component.

Paragraph 70. A biocatalyst comprising up to two or two or more SHC active site mutations and/or up to two or two or more site mutations other than SHC active site mutations wherein the active site mutations are selected from the active site amino acid positions (based on AacSHC) listed in Table 10 comprising active site positions 36, 168, 169, 261, 306, 307, 312, 365, 366, 420, 489, 600, 607, 609, and/or 612, preferably 169, 261, 312, 365, 489, 600, 609 and/or 612 and wherein the mutations other than active site mutations are selected from the site mutations listed in Table 7 and/or Table 8 and/or Table 9 and/or Table 18 disclosed herein, preferably comprising M132R and/or 1432T or functional equivalents, thereof wherein the biocatalyst is a protein, a host cell comprising the protein, a host cell lysate, freeze dried host cell, host cell extract or host cell fraction comprising the protein. By way of example, FIG. 19 of WO2016/170099 shows the location of the non-active site mutations identified in SHC/HAC variants 101A10, 111C8 and 215G2 (comprising M132R, 1432T and A224V) on the SHC Crystal Structure. A CAVER analysis of the AacSHC crystal structure can be performed to identify useful active site mutations and site mutations other than active site mutations useful for the preparation of a product of interest using an SHC variant comprising such beneficial active site mutations and site mutations other than active site mutations as described herein.

Paragraph 71. A process for preparing a composition comprising (−)-Ambrox or a mixture comprising (−)-Ambrox, comprising the following steps:
x) providing homofarnesol, a reaction medium and the biocatalyst of paragraph 70,
y) mixing the provided homofarnesol with the reaction medium and the biocatalyst of paragraph 22 and reacting the homofarnesol with the biocatalyst under reaction conditions so as to convert the homofarnesol to (−)-Ambrox and
z) obtaining a composition comprising (−)-Ambrox.

Paragraph 72. A process for preparing a composition comprising (−)-Ambrox or a mixture comprising (−)-Ambrox, comprising the following steps:
x) providing homofarnesol, a reaction medium and a biocatalyst disclosed in any one of Tables 2, 3, 7, 8, 9, 16 and/or 18
y) mixing the provided homofarnesol with the reaction medium and the biocatalyst disclosed in any one of Tables 2, 3, 7, 8, 9, 16 and/or 18 and reacting the homofarnesol with the biocatalyst under reaction conditions so as to convert the homofarnesol to (−)-Ambrox and
z) obtaining a composition comprising (−)-Ambrox.

Paragraph 73. A process for preparing a composition comprising Amberketal, or a mixture comprising Amberketal comprising the following steps:
x) providing hydroxyfarnesylacetone, a reaction medium and the biocatalyst disclosed in any one of Tables 2, 3, 7, 8, 9, 16 and/or 18
y) mixing the provided hydroxyfarnesylacetone with the reaction medium and the biocatalyst disclosed in any one of Tables 2, 3, 7, 8, 9, 16 and/or 18 and reacting the hydroxyfarnesylacetone with the biocatalyst under reaction conditions so as to convert the hydroxyfarnesylacetone to (−)-Amberketal and
z) obtaining a composition comprising Amberketal.

Paragraph 74. A process for preparing a composition comprising Ambra oxide or a mixture comprising Ambra oxide comprising the following steps:
x) providing (2-E)-Bishomofarnesol (BisEEH), a reaction medium and the biocatalyst disclosed in any one of Tables 2, 3, 7, 8, 9, 16 and/or 18
y) mixing the provided BisEEH with the reaction medium and the biocatalyst disclosed in any one of Tables 2, 3, 7, 8, 9, 16 and/or 18 and reacting the BisEEH with the biocatalyst under reaction conditions so as to convert the BisEEH to Ambra oxide and
z) obtaining a composition comprising Ambra oxide.

Paragraph 75: A process for preparing (−)-Ambrox or a mixture comprising (−)-Ambrox, wherein (3E,7E)-homofarnesol (EEH) or a mixture of stereoisomers comprising EEH is enzymatically converted to (−)-Ambrox or a mixture comprising (−)-Ambrox wherein the enzymatic conversion is carried out using an SHC/HAC enzyme under reaction conditions suitable for the production of (−)-Ambrox and wherein the mixture of stereoisomers comprising EEH consists essentially of homofarnesol isomers selected from one or more of the following groups consisting of [(3E,7E) and [(3Z,7E)] and/or [(3E,7E) and (3E,7Z)] and/or [(3Z,7E), (3E,7E) and (3E,7Z)] also designated as [EE:EZ], [EE:ZE] and [EE:EZ:ZE] respectively and wherein the SHC/HAC enzyme polypeptide sequence is selected from the group consisting of a SHC enzyme or SHC enzyme variant selected from Table 2, Table 3, Table 7, Table 8, Table 9, Table 16, or Table 18 and/or a sequence with a least 30%, at least 40% identity, at least 50% identity, or at least 60% identity, or at least 70% identity, or at least 80% identity, or at least 90% identity, or at least 95% identity, or at least 96% identity, or at least 97% identity, or at least 98% identity, or at least 99% identity to an SHC amino acid sequence or SHC variant amino acid sequence as selected from Table 2, Table 3, Table 7, Table 8, Table 9, Table 16, or Table 18.

### EXAMPLES

### Example 1

### Biocatalyst Production

For squalene hopene cyclase enzyme production in Escherichia coli the gene coding for the desired wild-type or variant SHC enzyme was inserted into plasmid pET-28a(+) (commercially available (for example from Merck Millipore) and as disclosed in SEQ ID NO:179 of FIG. 21 of WO 2016/170099), where it is under the control of an IPTG inducible T7 promotor. The plasmid was transformed into E. coli strain BL21(DE3) (commercially available) using a standard heat-shock transformation procedure.

### Cultivation Medium

The minimal medium used for biocatalyst production contained:
10% 10× citric acid/phosphate buffer (133 g/l KH₂PO₄; 40 g/l (NH₄)₂HPO₄, 17 g/l citric acid. H₂O in deionized water, with pH adjusted to 6.8 using 32% NaOH),
2.43% MgSO₄ solution (50% w/v MgSO₄·7H₂O in deionized water),
0.01% trace elements solution (50 g/l Na₂EDTA·2H₂O, 20 g/l FeSO₄·7H₂O, 3 g/l H₃BO₃, 0.9 g/l MnSO₄·2H₂O, 1.1 g/l CoCl₂, 80 g/l CuCl₂, 240 g/l NiSO₄·7H₂O, 100 g/l KI, 1.4 g/l (NH₄)₆Mo₇O₂₄·4H₂O, 1 g/l ZnSO₄·7H₂O in deionized water),
0.01% Thiamin solution (2.25 g/l Thiamin·HCl in deionized water),
2% glucose solution (20% w/v glucose in deionized water).

The citric acid/phosphate buffer was first sterilized by autoclaving, the other ingredients added afterwards from sterile solutions sterilized either by autoclaving or filter-sterilization (0.2 μm).

### Fermentation

Fermentations were run in 750 ml InforsHT reactors. To the fermentation vessel was added 168 ml deionized water. The reaction vessel was equipped with all required probes (pO₂, pH, sampling, antifoam), C+N feed and sodium hydroxide bottles and autoclaved. After autoclaving was added:
20 ml 10× phosphate/citric acid buffer
14 ml 50% glucose
0.53 ml MgSO₄ solution
2 ml (NH₄)₂SO₄ solution (50% (w/v) (NH₄)₂SO₄ in deionized water)
0.020 ml trace elements solution
0.400 ml thiamine solution
0.200 ml kanamycin solution (50 mg/ml)

The running parameters were as follows: pH=6.95, pO₂=40%, T=30° C., 300 rpm. Cascade: rpm 300, min 300, max 1000, flow (1/min) 0.1, min 0, max 0.6. Antifoam control: 1:9.

A seed culture was grown in LB medium (+Kanamycin) at 37° C., 220 rpm for 8 h. The fermenter was inoculated to an OD_{650 nm} of 0.4-0.5 from this seed culture. The fermentation was run first in batch mode for 11.5 h, where after was started the C+N feed with a feed solution (sterilized glucose solution (143 ml H₂O+35 g glucose) to which had been added after sterilization: 17.5 ml (NH₄)₂SO₄ solution, 1.8 ml MgSO₄ solution, 0.018 ml trace elements solution, 0.360 ml Thiamine solution, 0.180 ml kanamycin solution. The feed was run at a constant flow rate of approx. 4.2 ml/h. Glucose and NH₄⁺ measurements were done externally to evaluate availability of the C- and N-sources in the culture. Usually glucose levels stay very low.

Cultures were grown for a total of approx. 25 hours, where they reached typically and OD_{650 nm} of 40-45. SHC production was then induced by the addition of IPTG to a concentration of 1 mM to the fermenter, and lasted for approx. 16 h at 30° C. and pO₂=20%. At the end of induction, the cells were collected by centrifugation, washed with citric acid/sodium phosphate buffer pH 5.6 and stored as pellets at 4° C. or −20° C. until further use.

### Example 2

### Setting Optimized Reaction Conditions for SHC Variants

The reaction conditions for the SHC variants were individually optimized with regard to temperature, pH and SDS concentration. Biocatalysts were prepared by fermentation as described in Example 1 and used is whole cell reactions. The Homofarnesol feedstock used was of EEH:EZH ratio 80:20.

Reactions of 2-4 ml volume containing 4, 8 or 16 g/l E,E-Homofarnesol and cells loaded at an OD_{650 nm} of 10 were run in 0.1 M citric acid/sodium phosphate, or 0.1 M acetic acid/sodium acetate, or 0.1 M succinic acid/NaOH buffer pH 5.2-7.0, in presence of SDS at concentrations ranging from 0.005 to 0.095% at temperatures ranging from 25 to 55° C., at pH ranging from 5.2 to 6.6, and under constant agitation (Heidolph synthesis 1 Liquid 16 or 24 (800 rpm), or Radleys Carousel 12 Plus/Monoblock 16 (200 rpm)).

We discovered that AacSHC variants having several mutations exhibit an improved substrate specificity for EEH and/or a product selectivity for (−)-Ambrox which result in an improved EEH conversion rate. These mutations are:
Mutation W169G is introduced in P1 variants
Mutations W169G and G600M are introduced in P2 variants
Mutations W169G, A306V and G600M are introduced in P3 variants
(these positions correspond to positions in SEQ ID NO:1)

The mutations of the P1, P2 or P3 variants had been introduced in:
The wild type Aac SHC being represented by SEQ ID NO:1 leading to:
   AaC SHC P1 variant represented by SEQ ID NO: 4,
   AaC SHC P2 variant represented by SEQ ID NO: 5,
   AaC SHC P3 variant represented by SEQ ID NO: 6,
The SHC #65 variant being represented by SEQ ID NO:2 leading to:
   SHC #65 P1 variant represented by SEQ ID NO: 7,
   SHC #65 P2 variant represented by SEQ ID NO: 8,
   SHC #65 P3 variant represented by SEQ ID NO: 9,
The 215G2 SHC variant being represented by SEQ ID NO:3 leading to:
   215G2 SHC P1 variant represented by SEQ ID NO: 10,
   215G2 SHC P2 variant represented by SEQ ID NO: 11,
   215G2 SHC P3 variant represented by SEQ ID NO: 12,

Introducing mutations of the P1, P2, and P3 variants had a significant influence on enzymatic activity depending on the SHC variant considered. It can be assumed that the same effect may be expected for the other SHC variants as listed in Table 7 and/or Table 8. Reaction conditions defined as optimized were confirmed/adjusted (pH) in 0.1 M succinic acid/NaOH buffer, the buffer used in EEH bioconversion at high substrate concentration.

The results (Table 15) indicated the importance of setting properly reaction conditions to individual optima for a careful evaluation of the relative performance in E,E-Homofarnesol conversion of the SHC variants.

TABLE 15
Optimized reaction conditions for SHC variants.
Useful ranges and optimal values.

| | Temperature | | [SDS] |
|---|---|---|---|
| SHC enzyme | (° C.) | pH ¹ | (w/v %) ² |
| AacSHC ³ | 55 | 6.0 | 0.035 |
| AacSHC P1 | 40-45/45 | 6.0-6.4/6.2 | 0.005-0.050/0.035 |
| AacSHC P2 | 40-50/45 | 5.6-5.8/5.6 | 0.020-0.050/0.035 |
| AacSHC P3 | 45-50/50 | 5.8-6.6/6.4 | 0.005-0.030/0.020 |
| 215G2SHC ³ | 35 | 5.4 | 0.060 |
| 215G2SHC P1 | 35-40/35 | 6.2-6.6/6.4 | 0.020 |
| 215G2SHC P2 | 30-35/35 | 5.8-6.0/6.0 | 0.020 |
| 215G2SHC P3 | 35-40/40 | 6.0-6.6/6.4 | 0.035 |
| SHC#65 ⁴ | 45 | 5.6 | 0.050 |
| SHC#65 P2 | 40-45/40 | 5.4-6.4/5.6 | 0.030-0.040/0.030 |
| SHC#65 P3 | 45-50/45 | 5.4-6.2/5.8 | 0.010-0.030/0.025 |

¹ as determined in citric acid/sodium phosphate buffer,
² in reactions containing cells at an OD_{650 nm} of 10,
³ as published in WO2016/170099,
⁴ as published in WO2021/110848.

### Example 3

### Relative Performance of SHC Variants in E,E-Homofarnesol Cyclization Reactions

The influence of the mutations of P1, P2 and P3 variants on E,E-Homofarnesol conversion was investigated when introduced in wild type AacSHC and 215G2SHC background. Reactions were run in 4 ml volume with constant agitation (Heidolph Synthesis 1 Liquid 16, 800 rpm) applying individually optimized reaction conditions (T, pH, SDS). The Homofarnesol feedstock used was of EEH:EZH ratio 80:20.

With P1, P2 and P3 variants of AacSHC was EEH conversion dramatically increased over that of the parent wild type enzyme (see FIG. 1). P1, P2, and P3 variants of 215G2SHC showed a significantly increased EEH conversion over 215G2SHC (see FIG. 1).

### Example 4

Substrate specificity and product selectivity of P1, P2, and P3 SHC variants (homofarnesol) The influence of P1, P2 and P3 variants on substrate specificity and product selectivity was investigated when introduced in wild type AacSHC, 215G2SHC, and SHC #65. Reactions (4 ml volume), contained 8 g/l EEH and cells to an OD_{650 nm} of 10, and were run with constant agitation (Heidolph Synthesis 1 Liquid 16, 800 rpm) applying individually optimized reaction conditions (T, pH, SDS). The Homofarnesol feedstock used was a mixture of isomers with an EEH:EZH ratio of 80:20.

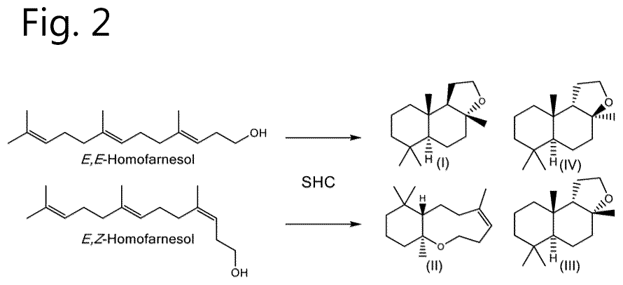

FIG. 2 shows the reaction products produced when Homofarnesol consisting of a mixture of EE and EZ isomers is cyclized with SHC as published in Eichhorn et al. Adv. Synth. Catal. (2018), 360, 2339-2351 and in WO2016/170099.

The analysis of the reaction products distribution in Homofarnesol cyclization reaction catalysed by wild type AacSHC, 215G2SHC and SHC #65, and in the P1, P2 and P3 variants of these enzymes showed an increased substrate specificity and product selectivity of the P1, P2, and P3 variants.

The parent enzymes converted both EEH and EZH to the products shown in FIG. 2.

The following conclusions could be drawn from FIG. 3: With the P1 variant of AacSHC was observed as strong reduction in compounds 1l, Ill, and IV in the reaction product, which indicated an increased specificity for E,E-Homofarnesol, E,Z-Homofarnesol being less efficiently cyclized. The product selectivity for (−)-Ambrox was increased as the main product of E,E-Homofarnesol cyclization was (−)-Ambrox, the reaction product containing no or almost no compound IV. With P2 and P3 variants of AacSHC the substrate specificity was slightly lower as more compound Ill was produced from E,Z-Homofarnesol cyclization, the product specificity for (−)-Ambrox remaining high, no or almost no compound IV being present in the reaction product. Overall, variant P1 showed highest substrate specificity, variants P2 and P3 showing increase E,Z-Homofarnesol conversion compared to variant P1.

The P1, P2, and P3 variants of 215G2SHC showed also a significantly increased substrate selectivity towards E,E-Homofarnesol and product selectivity towards (−)-Ambrox. As observed with the P1, P2, and P3 variants of AacSHC, 215G2 P2 and P3 showed lower substrate specificity compared to variant 215G2 SHC P1.

Introducing the mutations for P1, P2, and P3 variants in SHC #65 strongly increased substrate specificity towards E,E-Homofarnesol and product selectivity towards (−)-Ambrox, this selectivity remaining almost same within the three SHC #65 variants.

### Example 5

### Performance of P1, P2, and P3 AacSHC Variants in 125 g/l E,E-Homofarnesol Bioconversion

Biocatalysts produced by fermentation of the E. coli strains transformed with the plasmid carrying the gene coding for the selected SHC enzyme were used in 125 g/l E,E-Homofarnesol bioconversions. The Homofarnesol stock used was of EEH:EZH ratio 80:20. The cell concentration of the cell suspensions used was determined by centrifuge an approx. 1 g aliquot of the cell suspension in a tabletop centrifuge (13'000 rpm), carefully discarding the supernatant, and weighing the mass of the cell pellet (triplicate). 25 ml reactions were run in Radleys Carousel Plus/Monoblock 16. They contained 125 g/l E,E-Homofarnesol, 250 g/l cells, and were run in 0.1 M succinic acid or citric acid/sodium phosphate buffer. Temperature, pH, and SDS concentration (SDS:cells ratio) were set according to the conditions defined individually as optimal for each variant. The pH of the reaction was regularly measured at an externally calibrated pH electrode and adjusted manually with appropriately diluted phosphoric acid as required.

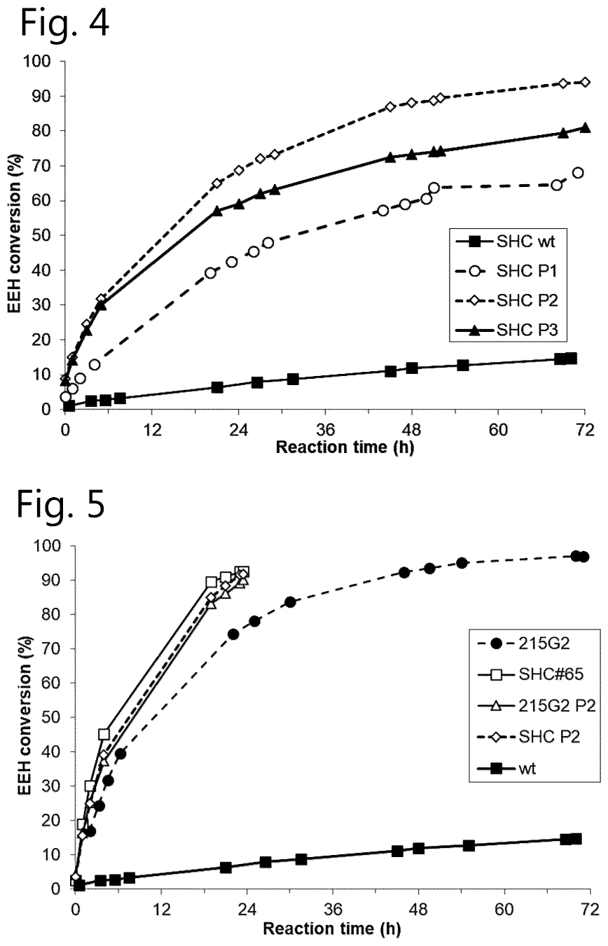

FIG. 4 demonstrate that the EEH conversion rate of the variants was markedly increased (from 50% to 90% depending on the variant) with compared to that of the parent wild type AacSHC enzyme (15%).

### Example 6

### Performance of 215G2 P2 and SHC #65 P2 Variants in 125 g/l E,E-Homofarnesol Bioconversion

Biocatalysts produced by fermentation of the E. coli strains transformed with the plasmid carrying the gene coding for the selected SHC enzyme were used in 125 g/l E,E-Homofarnesol bioconversions. The Homofarnesol stock used was of EEH:EZH ratio 80:20. The cell concentration of the cell suspensions used was determined by centrifuge an approx. 1 g aliquot of the cell suspension in a tabletop centrifuge (13'000 rpm), carefully discarding the supernatant, and weighing the mass of the cell pellet (triplicate).

25 ml reactions were run in Radleys Carousel Plus/Monoblock 16. They contained 125 g/l E,E-Homofarnesol, 250 g/l cells, and were run in 0.1 M succinic acid or citric acid/sodium phosphate buffer under conditions defined as optimal regarding temperature, pH, and SDS concentration (SDS:cells ratio). The pH of the reaction was measured at an externally calibrated pH electrode and adjusted manually with appropriately diluted phosphoric acid as required.

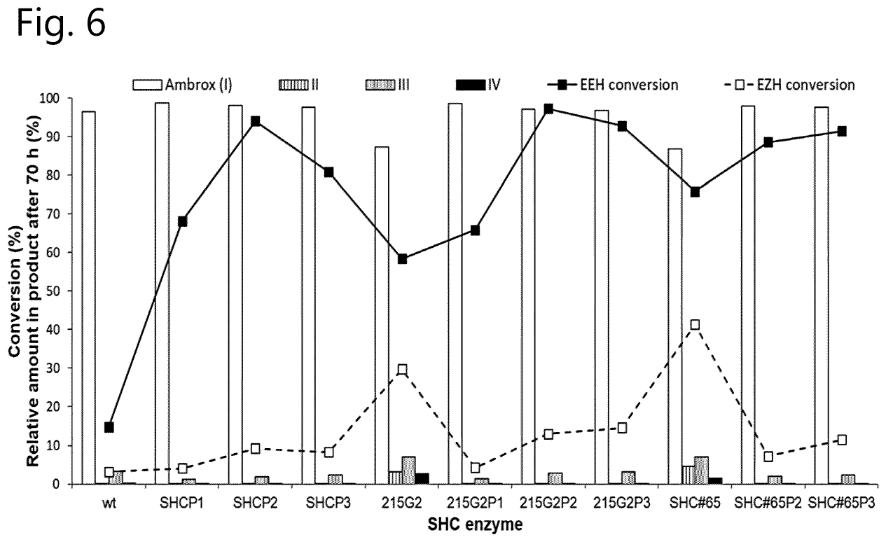

The introduction of P2 variant mutations into 215G2SHC dramatically improved EEH conversion: <90% conversion was obtained in 24 h instead of approx. 48 h with the parent 215G2SHC enzyme. Variants 215G2 P2 and SHC P2 performed as well as the SHC variant SHC #65 (see FIG. 5).

### Example 7

### Substrate Specificity and Product Selectivity of P1, P2, and P3 SHC Variants (Homofarnesol)

Biocatalysts produced by fermentation of the E. coli strains transformed with the plasmid carrying the gene coding for the selected SHC enzyme were used in 125 g/l E,E-Homofarnesol bioconversions. The Homofarnesol stock used was of EEH:EZH ratio 80:20. 25 ml reactions were run in Radleys Carousel Plus/Monoblock 16. The cell concentration of the cell suspensions used was determined by centrifuge an approx. 1 g aliquot of the cell suspension in a tabletop centrifuge (13'000 rpm), carefully discarding the supernatant, and weighing the mass of the cell pellet (triplicate).

The reactions contained 125 g/l E,E-Homofarnesol, 250 g/l cells, and were run under conditions defined as optimal regarding temperature, pH, and SDS concentration (SDS:Cells ratio) in 0.1 M succinic acid or citric acid/sodium phosphate buffer. The pH of the reaction was measured at an externally calibrated pH electrode and adjusted manually with appropriately diluted phosphoric acid as required.

FIG. 6 shows the composition of the reaction product, E,E-Homofarnesol and E,Z-Homofarnesol conversion with each of the variants tested. Introducing the mutations for P1, P2, and P3 variants into 215G2SHC and SHC #65 strongly reduced the conversion of E,Z-Homofarnesol increasing hereby the substrate selectivity of the variant enzymes towards E,E-Homofarnesol. The mutations also strongly increased the product specificity of the enzyme variants: no or almost no compound IV was produced from E,E-Homofarnesol, and compound Ill being the only or almost only product of E,Z-Homofarnesol cyclization.

### Example 8

### Bioconversion at Increased E,E-Homofarnesol Concentration

The Homofarnesol stock used was of EEH:EZH ratio 80:20. The cell concentration of the cell suspensions used was determined by centrifuge an approx. 1 g aliquot of the cell suspension in a tabletop centrifuge (13'000 rpm), carefully discarding the supernatant, and weighing the mass of the cell pellet (triplicate). 20 ml reactions were run in Radleys Carousel Plus/Monoblock 16 (constant agitation). They contained 250 g/l E,E-Homofarnesol, 250 g/l cells that had produced SHC variants SHC #65 or SHC #65 P2, and were run in 0.1 M succinic acid or citric acid/sodium phosphate buffer under conditions defined as optimal regarding temperature, pH, and SDS concentration (SDS:cells ratio). The pH of the reactions was measured at an externally calibrated pH electrode and adjusted manually with appropriately diluted phosphoric acid as required.

Although approximately the same level of E,E-Homofarnesol conversion was achieved with both SHC #65 and SHC #65 P2 variants, the increased substrate selectivity of SHC #65 towards E,E-Homofarnesol allowed for a faster conversion compared to SHC #65.

### Example 9

Reducing Cells:Substrate Ratio with SHC #65 and SHC #65 P2 SHC Variants

The Homofarnesol stock used was of EEH:EZH ratio 80:20. The cell concentration of the cell suspensions used was determined by centrifuge an approx. 1 g aliquot of the cell suspension in a tabletop centrifuge (13'000 rpm), carefully discarding the supernatant, and weighing the mass of the cell pellet (triplicate).

Reactions with SHC variant SHC #65 were run in 300 ml volume in a DASBox Fermenter (constant agitation), reactions with SHC #65 P2 SHC variant were run in 20 ml reactions in Radleys Carousel Plus/Monoblock 16 flasks (constant agitation). They contained either 250 g/l E,E-Homofarnesol and 150, 125, or 100 g/l cells that had produced SHC variants SHC #65 P2, or 300 g/l E,E-Homofarnesol and 300, 270 or 220 g/l cells that had produced SHC #65. The reactions were run in 0.1 M succinic acid (SHC #65) or citric acid/sodium phosphate (SHC #65 P2) buffer under conditions defined as optimal regarding temperature, pH, and SDS concentration (SDS:cells ratio). The pH of the reaction was measured at an externally calibrated pH electrode and adjusted manually with appropriately diluted phosphoric acid as required.

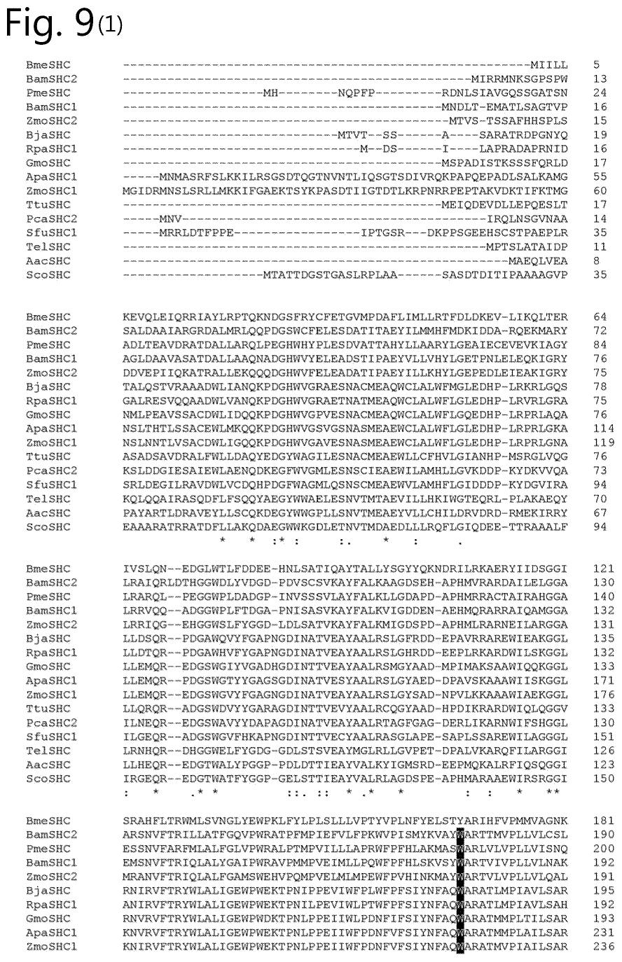

The results (see FIG. 8) indicated that it was possible to convert approx. 1.4 g E,E-Homofarnesol per gram of cells producing SHC variant SHC #65. When using cells producing SHC #65 P2 SHC variant, this value was increased to approx. at least 2.2 g E,E-Homofarnesol per gram of cells. This can be attributed to the increased substrate selectivity of SHC #65 P2 towards E,E-Homofarnesol.

In a second set of investigations, bioconversions were run as described above with 250 g/l EEH, and with cells that had produced SHC #65 P2 variant; the [cells]:[EEH] ratio was lowered further down to values of 0.3, 0.2, and 0.1. The reaction time was extended to approx. 200 h to possibly allow for reaching a plateau in E,E-Homofarnesol conversion. Approx. 98% EEH conversion was obtained with a [cells]:[EEH] ratio of 0.3, approx. 84% EEH conversion with a [cells]:[EEH] ratio of 0.2, and approx. 59% with a [cells]:[EEH] of 0.1. The plateau reached in the reaction with a [cells]:[EEH] ratio of 0.1 indicated the possibility of converting approx. 5.9 g of E,E-Homofarnesol per gram of cells under the reaction conditions applied. This represents 2.7-fold improvement over SHC #65.

### Example 10

GC-FID analysis Samples were extracted with an appropriate volume of tert-butylmethyl ether (MBTE/tBME) for quantification of their content in substrate and reaction products. The solvent fraction was separated from the water phase by centrifugation prior to analysis with gas chromatography. 1 μl of the solvent phase was injected (split ratio 10) onto a 30 m×0.32 mm×0.25 μm Zebron ZB-5 column. The column was developed at constant flow (4 ml/min H₂) with the temperature gradient: 100° C., 15° C./min to 200° C., 120° C./min to 240° C., 4 min at 240° C. Inlet temperature: 250° C., detector temperature: 250° C. This resulted in separation of EEH and EZH, and of the peaks corresponding to (−)-Ambrox and the 3 other reaction products resulting from E,E- and E,Z-Homofarnesol cyclization.

E,E-Homofarnesol and E,Z-Homofarnesol conversion were calculated from the areas of the peaks corresponding to the EEH or EZH cyclization products and EEH or EZH substrate peaks area with the following formulae:

$$\begin{matrix}EEH conversion ( \% ) = 100 \times ( {Area}_{EEH products} / ( {Area}_{EEH products} + {Area}_{EEH} ) ) \\ EZH conversion ( \% ) = 100 \times ( {Area}_{EZH products} / ( {Area}_{EZH products} + {Area}_{EZH} ) )\end{matrix}$$

### Example 11

### Squalene Hopene Cyclase Amino Acid Sequence Alignment

Source microorganisms and amino acid sequence accession numbers for squalene hopene cyclase enzymes are listed in Table 16.

TABLE 16
Squalene hopene cyclase enzymes. Source microorganisms
and amino acid sequence accessionnumbers. Accession
number is Genbank when not otherwise mentioned.

| Abbrevia- | | Accessionnum- | SEQ |
|---|---|---|---|
| tion | Source microorganism | ber | ID NO |
| AacSHC | Alicyclobacillus acidocaldarius | ACV59449.1 | 1 |
| ApaSHC1 | Acetobacter pasteurianus | ASC07046.1 | 24 |
| BamSHC1 | Burkholderia ambifaria | AJY25620.1 | 28 |
| BamSHC2 | Burkholderia ambifaria | ABI91648.1 | 29 |
| BjaSHC | Bradyrhizobium japonicum | ABQ33590.1 | 16 |
| (or | | | |
| BjpSHC) | | | |
| BmeSHC | Bacillus megaterium | ADF38987.1 | 25 |
| GmoSHC | Gluconobacter morbifer | EHH69691.1 | 22 |
| PcaSHC2 | Pelobacter carbinolicus or | ABA87615.1 | 30 |
| | Syntrophotalea carbinolica | | |
| | DSM 2380 | | |
| PmeSHC | Pseudomonas mendocina | A0A0X1SYK9¹ | 296 |
| RpaSHC1 | Rhodopseudomonas palustris | ABJ08391.1 | 31 |
| ScoSHC | Streptomyces coelicolor | CAB39697.1 | 32 |
| SfuSHC1 | Syntrophobacter fumaroxidans | ABK17672.1 | 33 |
| TelSHC | Thermosynechococcus elongatus | BAC09861.1 | 23 |
| TtuSHC | Teredinibacter turnerae | ACR13362.1 | 34 |
| ZmoSHC1 | Zymomonas mobilis | AVZ42714.1 | 13 |
| ZmoSHC2 | Zymomonas mobilis | AHJ71916.1 | 15 |

¹UniProtKB

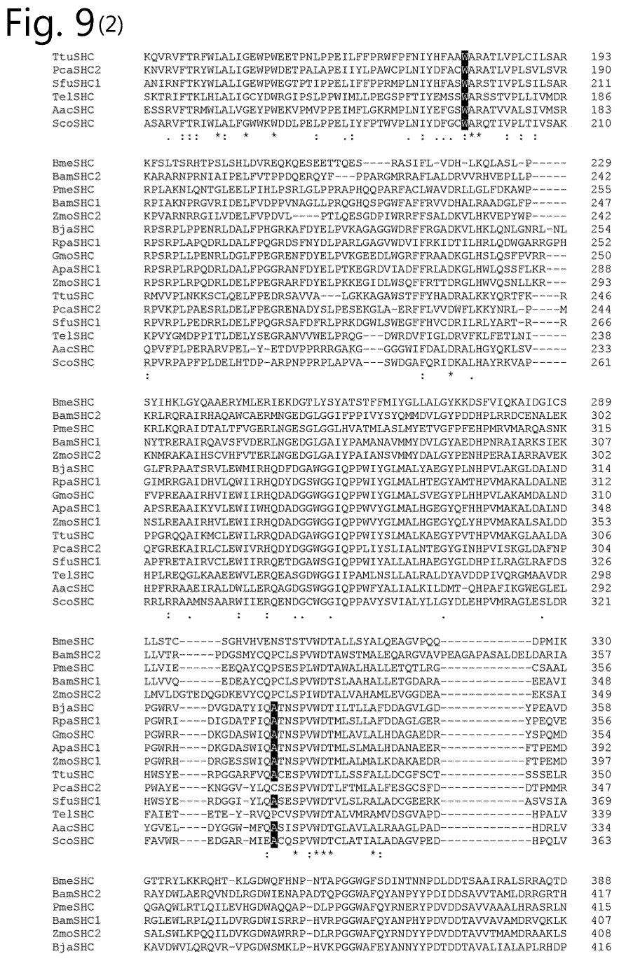

FIG. 9 shows an amino acid sequence alignment of the squalene hopene cyclase enzymes listed in Table 3 prepared with CLUSTAL O (1.2.4). The amino acids at positions 169, 306, and 600 of the Alicyclobacillus acidocaldarius (AacSHC) sequence are highlighted in white on a black background. W at position 169 is almost strictly conserved as is G at position 600 throughout the amino acid sequences aligned: 15 out of 16 sequences, one single conservative substitution.

Amino acids at position 306 are less conserved (9 out of 16 sequences).

### Example 12

### Substrate Specificity and Product Selectivity of P2 SHC Variants (Bis-Homofarnesol)

The influence of P2 mutations on substrate specificity and product selectivity was investigated when introduced in SHC #65 for the cyclization of Bis-homofarnesol for Ambra oxide production. The EE:EZ ratio of the Bis-homofarnesol feedstock used was 66:34.

Reactions (3 ml volume), contained 4 g/l E,E-Bis-homofarnesol and cells to an OD_{650 nm} of 10. The reactions were run with constant agitation (Heidolph Synthesis 1 Liquid 24, 800 rpm). The reaction with SHC #65 was run at pH 5.6 and 45° C. in presence of 0.07% SDS. The reaction with SHC #65 P2 was run at pH 6.2 and 35° C. in presence of 0.03% SDS.

The reactions were sampled over time, extracted with MTBE and their content in E,E-Bis-homofarnesol, E,Z-Bis-homofarnesol, compounds X, XI, XII, and XIII (reaction products as shown in Table 12) analysed by GC-FID. Conversion was calculated as indicated in Example 10 for Homofarnesol.

The new variants comprising P2 mutations in the SHC #65 background exhibit increased conversion of E,E-Bis-homofarnesol (see FIG. 10).

A higher specificity for the E,E isomer of Bis-homofarnesol and a higher product specificity towards Ambra oxide of the SHC #65 P2 variant was observed compared to SH #65. E,E-Bis-homofarnesol was preferentially converted over E,Z-Bis-homofarnesol with SHC #65 P2. The proportion of Ambra oxide (X) in the reaction products arising from E,E-Bis-homofarnesol cyclization (X+XII) was increased: approx. 95:5 X:XII with SHC #65 P2 compared to approx. 90:10 with SHC #65 (Table 17).

Product specificity regarding E,Z-Bis-homofarnesol cyclization was increased as well as no or almost no compound XIII resulted from E,Z-Bis-homofarnesol cyclization, but only compound XI was detected (Table 17).

Overall the observations regarding substrate specificity and product selectivity were in line with the ones described with Homofarnesol in other examples. It was concluded that the effect of P2 mutations can be transferred to substrates other than E,E-homofarnesol, E,E-Bis-homofarnesol being one example. It can be assumed that the benefits obtained here can be transferred to other substrates (eg for the cyclisation of hydroxyfarnesylacetone for the production of Amberketal as set out in WO2021/209482).

TABLE 17
Bis-homofarnesol cyclization with SHC enzyme variants. Bis-
homofarnesol was cyclized with SHC#65 and SHC#65
P2. The composition of the reaction product (mixture of
compounds X, XI, XII, XIII) is indicated for each compound
as percent of total reaction product, and as a sum of the
product pairs resulting form E,E-Bis-homofarnesol (X +
XII) and E,Z-Bis-homofarnesol (XI + XIII).

| | Bis- | Reaction products | | | | | |
|---|---|---|---|---|---|---|---|
| | homofarnesol | (% of total reaction product) | | | | | |
| SHC | conversion | | | | | X + | XI + |
| enzyme | (%) ¹ | X | XI | XII | XIII | XII | XIII |
| SHC#65 | 85.1 | 70.5 | 17.0 | 8.2 | 4.3 | 78.7 | 21.3 |
| SHC#65 P2 | 86.5 | 80.9 | 14.7 | 4.4 | 0.0 | 85.3 | 14.7 |

¹ Total conversion of both EE and EZ isomers after 24 h of reaction.

### Example 13

### Influence of P1 Mutation on Homofarnesol Conversion and Product Selectivity in Wt SHC Enzymes.

The influence of P1 mutation on Homofarnesol conversion and product selectivity for (−)-Ambrox was investigated when introduced in AacSHC (SEQ ID NO:1), ZmoSHC1 (SEQ ID NO:13 ZmoSHC2 (SEQ ID NO:15), TeISHC (SEQ ID NO:23), and ScoSHC (SEQ ID NO:32).
AaC SHC P1 variant (W169G mutation) represented by SEQ ID NO: 4,
ZmoSHC1 P1 variant (W222G mutation) represented by SEQ ID NO:312
ZmoSCH2 P1 variant (W177G mutation) represented by SEQ ID NO:313
TeISHC P1 variant (W172G mutation) represented by SEQ ID NO: 304
ScoSHC P1 variant (W196G mutation) represented by SEQ ID NO:311

Reactions (0.5 ml volume) contained 2.36 g/l Homofarnesol, cells that had produced the SHC enzyme to an OD_{650 nm} of 6 and were run in deionized water at 30° C. (constant agitation). They were solvent extracted after 24 h and their content in reaction products (as shown in Table 13 and E,E- and E,Z-Homofarnesol isomers analysed by GC-FID analysis.

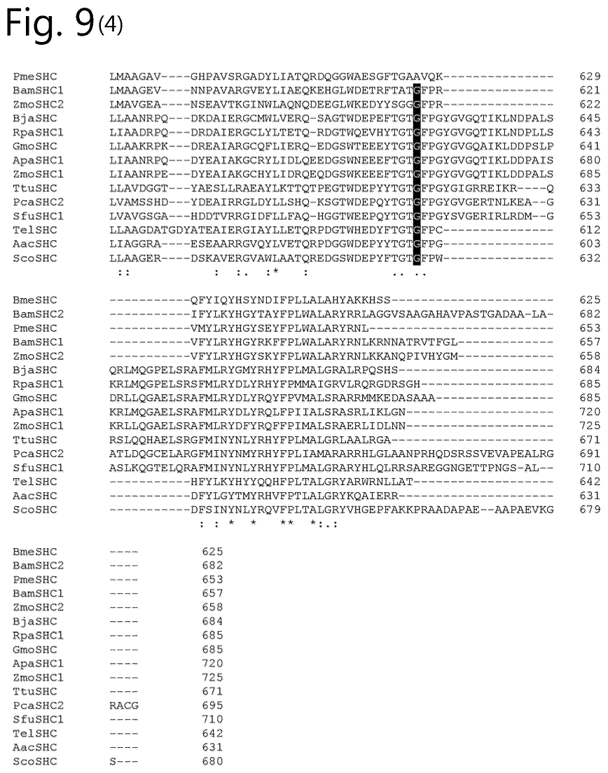

The results indicated an increase in total homofarnesol conversion when the P1 mutation was inserted into the wild type SHC enzymes tested (FIG. 11).

With the reaction conditions applied was the selectivity of the wild type SHC enzymes towards E,E-homofarnesol already quite high. It was nevertheless possible to conclude on an increased selectivity of the corresponding P1 variants towards E,E-Homofarnesol, this increase in selectivity being more pronounced with TeISHC P1, and significant with regard to ScoSHC P1 in comparison to the corresponding wild-type enzyme (FIG. 11).

### Example 14

The table below is identical with Table 10 earlier depicted. This table shows a set of conserved amino acids at the active site for five wild type SHC enzymes

TABLE 10
Set of conserved amino acids at the active site of 5 wt
SHC enzymes. Conserved positions can be seen for amino
acid positions 169, 261, 312, 365, 489, 600, 609 and 612
for five different WT SHC enzymes. Differences in hydrogen
binding pocket can be seen for Q, Y and C in ZmoSHC1,
ZmoSHC2 and ScoSHC for the amino acid position 168.

| active site | | | | | |
|---|---|---|---|---|---|
| amino acid | Sequence identity to AacSHC (%) | | | | |
| position based | 0 | 45.8 | 38.3 | 44.2 | 51.0 |
| on AacSHC | AacSHC | ZmoSHC1 | ZmoSHC2 | TelSHC | ScoSHC |
| 36 | L | V | L | L | L |
| 168 | S | Q | Y | S | C |
| 169 | W | W | W | W | W |
| 261 | I | I | I | I | I |
| 306 | A | A | P | P | A |
| 307 | S | T | C | C | C |
| 312 | W | W | W | W | W |
| 365 | F | F | F | F | F |
| 366 | Q | E | Q | E | E |
| 420 | Y | F | F | F | F |
| 489 | W | W | W | W | W |
| 600 | G | G | G | G | G |
| 607 | L | L | L | L | I |
| 609 | Y | Y | Y | Y | Y |
| 612 | Y | Y | Y | Y | Y |

conserved (bold font)
difference in hydrogen-binding pocket (italic font)

### Example 15

### Substrate Specificity and Product Selectivity of Additional SHC Variants (Eg SEQ ID NO: 306, or a Combination of the Mutations in SEQ ID NO: 5 and SEQ ID NO: 369)

The mutations of the P1, P2 or P3 variants are introduced in:
The wild type Aac SHC being represented by SEQ ID NO:1 leading to:
   AaC SHC P4 variant represented by SEQ ID NO: 5 and SEQ ID NO: 369
   AaC SHC P4 variant represented by SEQ ID NO:306

The introduction of the P4 variants show a significant influence on enzymatic activity. Reaction conditions defined as optimized are confirmed/adjusted (pH) in 0.1 M succinic acid/NaOH buffer, the buffer used in EEH bioconversion either at low or high substrate concentration. Overall the observations regarding substrate specificity and product selectivity are in line with the results described in other examples.

### Example 16

Reducing Cells:Substrate Ratio with SHC #65 P2 SHC at Increased EEH Concentration

The EE:EZ ratio of the Homofarnesol stock used was 80:20. The cell concentration of the cell suspension used was determined as outlined in Example 9.

Reactions were run in a total volume of 25 g (“25 ml reactions”) in Radleys Monoblock Flask (200 rpm, constant agitation) using cells that had produced SHC variant SHC #65 P2. They contained 400 g/l E,E-Homofarnesol and the appropriate amount of cells for a [cells]:[EEH] ratio of 0.6, 0.5, 0.4, or 0.3. The reactions were run at pH 5.8 and 35° C. in 0.1 M succinic acid buffer with SDS provided at a constant [SDS]:[cells] ratio of 0.025. The pH of the reactions was measured at an externally calibrated electrode and adjusted manually as required with appropriately diluted phosphoric acid.

With an EEH concentration of 400 g/l, full EEH conversion was achieved with a [cells]:[EEH]0.5 in 72 hours. With a [cells]:[EEH] ratio of 0.4, 95% EEH conversion was obtained in the same time. It was deduced from the reaction run at a [cells]:[EEH] ratio of 0.3 (approx. 70% EEH conversion after 3 days) that at least 2.3 g of EEH could be converted per gram of cells under the conditions tested.

### Example 17

### Increased Volumetric Productivity

The EE:EZ ratio of the Homofarnesol stock used was 80:20. The cell concentration of the cell suspension used was determined as outlined in Example 9.

Reactions were run in a total volume of 25 g (“25 ml reactions”) in Radleys Monoblock Flask (200 rpm, constant agitation) using cells that had produced SHC variant SHC #65 P2. They contained either 450 g/l E,E-Homofarnesol and 180 g/l cells ([cells]:[EEH] ratio of 0.4), or 250 g/l g/l E,E-Homofarnesol and 250 g/l cells ([cells]:[EEH] ratio of 1.0). The reactions were run in 0.1 M succinic acid buffer at pH 5.8 and 35° C., and at a constant [SDS]:[cells] ratio of 0.030. The pH of the reactions was measured at an externally calibrated pH electrode and adjusted as required manually with appropriately diluted phosphoric acid.

With 450 g/l EEH and a [cells]:[EEH] ratio of 0.4, full EEH conversion was obtained in 72 h. Full conversion was obtained in 24 h with 250 g/l EEH at [cells]:[EEH] ratio of 1.0. This indicated the possibility for full conversion of up to 450 g/l EEH in 3 days using an appropriate amount of cells, and that this was possible with a [cells]:[EEH] ratio at least as low as 0.4 under the conditions tested.

### Example 18

### Preparative Scale Reaction: Bioconversion at Increased EEH Concentration

The EE:EZ ratio of the Homofarnesol stock used was 80:20. The cell concentration of the cell suspension used was determined as outlined in Example 9.

Reactions were run in a total volume of 1.1 kg (“1.1 litre reaction”) in a 1.5 litre sulfonation flask (230 rpm, constant agitation) placed in a water bath. The reaction contained 400 g/l E,E-Homofarnesol and 200 g/l cells that had produced SHC variant SHC #65 P2 ([cells]:[EEH] ratio of 0.5). The reaction was run at 35° C. and pH 5.8 in 0.1 M succinic acid buffer in presence of 5 g/l SDS ([SDS]:[cells] ratio of 0.025). The pH of the reaction was measured at an externally calibrated pH electrode and adjusted as required manually with appropriately diluted phosphoric acid.

EEH conversion was >97.1% after 72 hours as judged from GC-FID analysis results. The reaction broth was treated as described e.g. in WO 2017182542 or WO2022023464 prior to filtration over a 500 mesh filter. The filter cake was washed with deionized water and dried under vacuum (approx. 80 mbar, 60° C.). 428.6 g crude (−)-ambrox were recovered (white-brownish, slightly sticky crystalline material). GC-FID analysis of the crude material indicated that it consisted of 90.7% (−)-ambrox, 0.0% compound II, 0.0% compound Ill, 1.2% compound IV, 2.6% E,E-Homofarnesol, and 5.5% E,Z-Homofarnesol. According to this data (−)-ambrox accounted for 98.7% of the reaction products mixture ((−)-ambrox and compounds 1l, Ill, and IV). Overall (−)-ambrox recovery yield was 91.0%: 388.7 g recovered form 427.2 g expected (EEH conversion as determined by GC-FID).

Additional sequences

>ZmoSHC1 P2: SEQ ID NO: 353
MGIDRMNSLSRLLMKKIFGAEKTSYKPASDTIIGTDTLKRPNRRPEPTAKVDKTIFKTMGNSLNNTLVSA
CDWLIGQQKPDGHWVGAVESNASMEAEWCLALWFLGLEDHPLRPRLGNALLEMQREDGSWGVYFGAGNGD
INATVEAYAALRSLGYSADNPVLKKAAAWIAEKGGLKNIRVFTRYWLALIGEWPWEKTPNLPPEIIWFPD
NFVFSIYNFAQGARATMVPIAILSARRPSRPLRPQDRLDELFPEGRARFDYELPKKEGIDLWSQFFRTTD
RGLHWVQSNLLKRNSLREAAIRHVLEWIIRHQDADGGWGGIQPPWVYGLMALHGEGYQLYHPVMAKALSA
LDDPGWRHDRGESSWIQATNSPVWDTMLALMALKDAKAEDRFTPEMDKAADWLLARQVKVKGDWSIKLPD
VEPGGWAFEYANDRYPDTDDTAVALIALSSYRDKEEWQKKGVEDAITRGVNWLIAMQSECGGWGAFDKDN
NRSILSKIPFCDFGESIDPPSVDVTAHVLEAFGTLGLSRDMPVIQKAIDYVRSEQEAEGAWFGRWGVNYI
YGTGAVLPALAAIGEDMTQPYITKACDWLVAHQQEDGGWGESCSSYMEIDSIGKGPTTPSQTAWALMGLI
AANRPEDYEAIAKGCHYLIDRQEQDGSWKEEEFTGTMFPGYGVGQTIKLDDPALSKRLLQGAELSRAFML
RYDFYRQFFPIMALSRAERLIDLNN
>ZmoSHC1 P3: SEQ ID O: 354
MGIDRMNSLSRLLMKKIFGAEKTSYKPASDTIIGTDTLKRPNRRPEPTAKVDKTIFKTMGNSLNNTLVSA
CDWLIGQQKPDGHWVGAVESNASMEAEWCLALWFLGLEDHPLRPRLGNALLEMQREDGSWGVYFGAGNGD
INATVEAYAALRSLGYSADNPVLKKAAAWIAEKGGLKNIRVFTRYWLALIGEWPWEKTPNLPPEIIWEPD
NFVFSIYNFAQGARATMVPIAILSARRPSRPLRPQDRLDELFPEGRARFDYELPKKEGIDLWSQFFRTTD
RGLHWVQSNLLKRNSLREAAIRHVLEWIIRHQDADGGWGGIQPPWVYGLMALHGEGYQLYHPVMAKALSA
LDDPGWRHDRGESSWIQVTNSPVWDTMLALMALKDAKAEDRETPEMDKAADWLLARQVKVKGDWSIKLPD
VEPGGWAFEYANDRYPDTDDTAVALIALSSYRDKEEWQKKGVEDAITRGVNWLIAMQSECGGWGAFDKDN
NRSILSKIPFCDFGESIDPPSVDVTAHVLEAFGTLGLSRDMPVIQKAIDYVRSEQEAEGAWFGRWGVNYI
YGTGAVLPALAAIGEDMTQPYITKACDWLVAHQQEDGGWGESCSSYMEIDSIGKGPTTPSQTAWALMGLI
AANRPEDYEAIAKGCHYLIDRQEQDGSWKEEEFTGTMFPGYGVGQTIKLDDPALSKRLLQGAELSRAFML
RYDFYRQFFPIMALSRAERLIDLNN
>ZmoSHC2 P2: SEQ ID NO: 355
MTVSTSSAFHHSPLSDDVEPIIQKATRALLEKQQQDGHWVFELEADATIPAEYILLKHYLGEPEDLEIEA
KIGRYLRRIQGEHGGWSLFYGGDLDLSATVKAYFALKMIGDSPDAPHMLRARNEILARGGAMRANVFTRI
QLALFGAMSWEHVPQMPVELMLMPEWFPVHINKMAYGARTVLVPLLVLQALKPVARNRRGILVDELFVPD
VLPTLQESGDPIWRRFFSALDKVLHKVEPYWPKNMRAKAIHSCVHFVTERLNGEDGLGAIYPAIANSVMM
YDALGYPENHPERAIARRAVEKLMVLDGTEDQGDKEVYCQPCLSPIWDTALVAHAMLEVGGDEAEKSAIS
ALSWLKPQQILDVKGDWAWRRPDLRPGGWAFQYRNDYYPDVDDTAVVTMAMDRAAKLSDLHDDFEESKAR
AMEWTIGMQSDNGGWGAFDANNSYTYLNNIPFADHGALLDPPTVDVSARCVSMMAQAGISITDPKMKAAV
DYLLKEQEEDGSWFGRWGVNYIYGTWSALCALNVAALPHDHLAVQKAVAWLKTIQNEDGGWGENCDSYAL
DYSGYEPMDSTASQTAWALLGLMAVGEANSEAVTKGINWLAQNQDEEGLWKEDYYSGGMFPRVFYLRYHG
YSKYFPLWALARYRNLKKANQPIVHYGM
>ZmoSHC2 P3: SEQ ID NO: 356
MTVSTSSAFHHSPLSDDVEPIIQKATRALLEKQQQDGHWVFELEADATIPAEYILLKHYLGEPEDLEIEA
KIGRYLRRIQGEHGGWSLFYGGDLDLSATVKAYFALKMIGDSPDAPHMLRARNEILARGGAMRANVFTRI
QLALFGAMSWEHVPQMPVELMLMPEWFPVHINKMAYGARTVLVPLLVLQALKPVARNRRGILVDELFVPD
VLPTLQESGDPIWRRFFSALDKVLHKVEPYWPKNMRAKAIHSCVHFVTERLNGEDGLGAIYPAIANSVMM
YDALGYPENHPERAIARRAVEKLMVLDGTEDQGDKEVYCQVCLSPIWDTALVAHAMLEVGGDEAEKSAIS
ALSWLKPQQILDVKGDWAWRRPDLRPGGWAFQYRNDYYPDVDDTAVVTMAMDRAAKLSDLHDDFEESKAR
AMEWTIGMQSDNGGWGAFDANNSYTYLNNIPFADHGALLDPPTVDVSARCVSMMAQAGISITDPKMKAAV
DYLLKEQEEDGSWFGRWGVNYIYGTWSALCALNVAALPHDHLAVQKAVAWLKTIQNEDGGWGENCDSYAL
DYSGYEPMDSTASQTAWALLGLMAVGEANSEAVTKGINWLAQNQDEEGLWKEDYYSGGMFPRVFYLRYHG
YSKYFPLWALARYRNLKKANQPIVHYGM
>TelSHC P2: SEQ ID NO: 359
MPTSLATAIDPKQLQQAIRASQDELFSQQYAEGYWWAELESNVTMTAEVILLHKIWGTEQRLPLAKAEQY
LRNHQRDHGGWELFYGDGGDLSTSVEAYMGLRLLGVPETDPALVKARQFILARGGISKTRIFTKLHLALI
GCYDWRGIPSLPPWIMLLPEGSPETIYEMSSGARSSTVPLLIVMDRKPVYGMDPPITLDELYSEGRANVV
WELPRQGDWRDVFIGLDRVFKLFETLNIHPLREQGLKAAEEWVLERQEASGDWGGIIPAMLNSLLALRAL
DYAVDDPIVQRGMAAVDRFAIETETEYRVQPCVSPVWDTALVMRAMVDSGVAPDHPALVKAGEWLLSKQI
LDYGDWHIKNKKGRPGGWAFEFENRFYPDVDDTAVVVMALHAVTLPNENLKRRAIERAVAWIASMQCRPG
GWAAFDVDNDQDWLNGIPYGDLKAMIDPNTADVTARVLEMVGRCQLAFDRVALDRALAYLRNEQEPEGCW
FGRWGVNYLYGTSGVLTALSLVAPRYDRWRIRRAAEWLMQCQNADGGWGETCWSYHDPSLKGKGDSTASQ
TAWAIIGLLAAGDATGDYATEAIERGIAYLLETQRPDGTWHEDYFTGTMFPCHFYLKYHYYQQHFPLTAL
GRYARWRNLLAT
>TelSHC P3: SEQ ID NO: 360
MPTSLATAIDPKQLQQAIRASQDELFSQQYAEGYWWAELESNVTMTAEVILLHKIWGTEQRLPLAKAEQY
LRNHQRDHGGWELFYGDGGDLSTSVEAYMGLRLLGVPETDPALVKARQFILARGGISKTRIFTKLHLALI
GCYDWRGIPSLPPWIMLLPEGSPFTIYEMSSGARSSTVPLLIVMDRKPVYGMDPPITLDELYSEGRANVV
WELPRQGDWRDVFIGLDRVFKLFETLNIHPLREQGLKAAEEWVLERQEASGDWGGIIPAMLNSLLALRAL
DYAVDDPIVQRGMAAVDRFAIETETEYRVQVCVSPVWDTALVMRAMVDSGVAPDHPALVKAGEWLLSKQI
LDYGDWHIKNKKGRPGGWAFEFENRFYPDVDDTAVVVMALHAVTLPNENLKRRAIERAVAWIASMQCRPG
GWAAFDVDNDQDWLNGIPYGDLKAMIDPNTADVTARVLEMVGRCQLAFDRVALDRALAYLRNEQEPEGCW
FGRWGVNYLYGTSGVLTALSLVAPRYDRWRIRRAAEWLMQCQNADGGWGETCWSYHDPSLKGKGDSTASQ
TAWAIIGLLAAGDATGDYATEAIERGIAYLLETQRPDGTWHEDYFTGTMFPCHFYLKYHYYQQHEPLTAL
GRYARWRNLLAT
>ScoSHC P2: SEQ ID NO: 357
MTATTDGSTGASLRPLAASASDTDITIPAAAAGVPEAAARATRRATDELLAKQDAEGWWKGDLETNVTMD
AEDLLLRQFLGIQDEETTRAAALFIRGEQREDGTWATFYGGPGELSTTIEAYVALRLAGDSPEAPHMARA
AEWIRSRGGIASARVETRIWLALFGWWKWDDLPELPPELIYFPTWVPLNIYDFGCGARQTIVPLTIVSAK
RPVRPAPFPLDELHTDPARPNPPRPLAPVASWDGAFQRIDKALHAYRKVAPRRLRRAAMNSAARWIIERQ
ENDGCWGGIQPPAVYSVIALYLLGYDLEHPVMRAGLESLDRFAVWREDGARMIEACQSPVWDTCLATIAL
ADAGVPEDHPQLVKASDWMLGEQIVRPGDWSVKRPGLPPGGWAFEFHNDNYPDIDDTAEVVLALRRVRHH
DPERVEKAIGRGVRWNLGMQSKNGAWGAFDVDNTSAFPNRLPFCDFGEVIDPPSADVTAHVVEMLAVEGL
AHDPRTRRGIQWLLDAQETDGSWFGRWGVNYVYGTGSVIPALTAAGLPTSHPAIRRAVRWLESVQNEDGG
WGEDLRSYRYVREWSGRGASTASQTGWALMALLAAGERDSKAVERGVAWLAATQREDGSWDEPYFTGTME
PWDESINYNLYRQVFPLTALGRYVHGEPFAKKPRAADAPAEAAPAEVKGS
>ScoSHC P3: SEQ ID NO: 358
MTATTDGSTGASLRPLAASASDTDITIPAAAAGVPEAAARATRRATDELLAKQDAEGWWKGDLETNVTMD
AEDLLLRQFLGIQDEETTRAAALFIRGEQREDGTWATFYGGPGELSTTIEAYVALRLAGDSPEAPHMARA
AEWIRSRGGIASARVETRIWLALFGWWKWDDLPELPPELIYFPTWVPLNIYDFGCGARQTIVPLTIVSAK
RPVRPAPFPLDELHTDPARPNPPRPLAPVASWDGAFQRIDKALHAYRKVAPRRLRRAAMNSAARWIIERQ
ENDGCWGGIQPPAVYSVIALYLLGYDLEHPVMRAGLESLDRFAVWREDGARMIEVCQSPVWDTCLATIAL
ADAGVPEDHPQLVKASDWMLGEQIVRPGDWSVKRPGLPPGGWAFEFHNDNYPDIDDTAEVVLALRRVRHH
DPERVEKAIGRGVRWNLGMQSKNGAWGAFDVDNTSAFPNRLPFCDEGEVIDPPSADVTAHVVEMLAVEGL
AHDPRTRRGIQWLLDAQETDGSWFGRWGVNYVYGTGSVIPALTAAGLPTSHPAIRRAVRWLESVQNEDGG
WGEDLRSYRYVREWSGRGASTASQTGWALMALLAAGERDSKAVERGVAWLAATQREDGSWDEPYFTGTME
PWDESINYNLYRQVFPLTALGRYVHGEPFAKKPRAADAPAEAAPAEVKGS
AacSHC P1 variant: SEQ ID NO: 4
MAEQLVEAPAYARTLDRAVEYLLSCQKDEGYWWGPLLSNVTMEAEYVLLCHILDRVDRDRMEKIRRYLLHEQREDGT
WALYPGGPPDLDTTIEAYVALKYIGMSRDEEPMQKALRFIQSQGGIESSRVFTRMWLALVGEYPWEKVPMVPPEIMFL
GKRMPLNIYEFGSGARATVVALSIVMSRQPVFPLPERARVPELYETDVPPRRRGAKGGGGWIFDALDRALHGYQKLSV
HPFRRAAEIRALDWLLERQAGDGSWGGIQPPWFYALIALKILDMTQHPAFIKGWEGLELYGVELDYGGWMFQASISP
VWDTGLAVLALRAAGLPADHDRLVKAGEWLLDRQITVPGDWAVKRPNLKPGGFAFQFDNVYYPDVDDTAVVVWAL
NTLRLPDERRRRDAMTKGFRWIVGMQSSNGGWGAYDVDNTSDLPNHIPFCDFGEVTDPPSEDVTAHVLECFGSFGY
DDAWKVIRRAVEYLKREQKPDGSWFGRWGVNYLYGTGAVVSALKAVGIDTREPYIQKALDWVEQHQNPDGGWGE
DCRSYEDPAYAGKGASTPSQTAWALMALIAGGRAESEAARRGVQYLVETQRPDGGWDEPYYTGTGFPGDFYLGYTM
YRHVFPTLALGRYKQAIERR
AacSHC P2 variant: SEQ ID NO: 5
MAEQLVEAPAYARTLDRAVEYLLSCQKDEGYWWGPLLSNVTMEAEYVLLCHILDRVDRDRMEKIRRYLLHEQREDGT
WALYPGGPPDLDTTIEAYVALKYIGMSRDEEPMQKALRFIQSQGGIESSRVFTRMWLALVGEYPWEKVPMVPPEIMFL
GKRMPLNIYEFGSGARATVVALSIVMSRQPVFPLPERARVPELYETDVPPRRRGAKGGGGWIFDALDRALHGYQKLSV
HPFRRAAEIRALDWLLERQAGDGSWGGIQPPWFYALIALKILDMTQHPAFIKGWEGLELYGVELDYGGWMFQASISP
VWDTGLAVLALRAAGLPADHDRLVKAGEWLLDRQITVPGDWAVKRPNLKPGGFAFQFDNVYYPDVDDTAVVVWAL
NTLRLPDERRRRDAMTKGFRWIVGMQSSNGGWGAYDVDNTSDLPNHIPFCDFGEVTDPPSEDVTAHVLECFGSFGY
DDAWKVIRRAVEYLKREQKPDGSWFGRWGVNYLYGTGAVVSALKAVGIDTREPYIQKALDWVEQHQNPDGGWGE
DCRSYEDPAYAGKGASTPSQTAWALMALIAGGRAESEAARRGVQYLVETQRPDGGWDEPYYTGTMFPGDFYLGYTM
YRHVFPTLALGRYKQAIERR
AacSHC P3 variant: SEQ ID NO: 6
MAEQLVEAPAYARTLDRAVEYLLSCQKDEGYWWGPLLSNVTMEAEYVLLCHILDRVDRDRMEKIRRYLLHEQREDGT
WALYPGGPPDLDTTIEAYVALKYIGMSRDEEPMQKALRFIQSQGGIESSRVFTRMWLALVGEYPWEKVPMVPPEIMFL
GKRMPLNIYEFGSGARATVVALSIVMSRQPVFPLPERARVPELYETDVPPRRRGAKGGGGWIFDALDRALHGYQKLSV
HPFRRAAEIRALDWLLERQAGDGSWGGIQPPWFYALIALKILDMTQHPAFIKGWEGLELYGVELDYGGWMFQVSISP
VWDTGLAVLALRAAGLPADHDRLVKAGEWLLDRQITVPGDWAVKRPNLKPGGFAFQFDNVYYPDVDDTAVVVWAL
NTLRLPDERRRRDAMTKGFRWIVGMQSSNGGWGAYDVDNTSDLPNHIPFCDFGEVTDPPSEDVTAHVLECFGSFGY
DDAWKVIRRAVEYLKREQKPDGSWFGRWGVNYLYGTGAVVSALKAVGIDTREPYIQKALDWVEQHQNPDGGWGE
DCRSYEDPAYAGKGASTPSQTAWALMALIAGGRAESEAARRGVQYLVETQRPDGGWDEPYYTGTMFPGDFYLGYTM
YRHVFPTLALGRYKQAIERR
ZmoSHC1 P1 variant: SEQ ID NO: 312
MTRALRQAPESAGAIGIAAASPATETSGQDTHPREISGAITAARDALLKLQQADGHWCFMLEADCTIPAEYILWTHFTG
ELEPEIERKLAARLRAKQASHGGWPLYEGGDLDISCSVKVYYALKLVGDDPNAPHMRRAREAILAQGGGARANVFTRL
ALAMFSQIPWRGVPFIPVEIMLLPRWFPFHLSKVSYWSRTVMVPLAILYSLKAQAQNPRNVHIQELFTVPPEQERHYFP
VRSRLNKILLSVERTARLLEPLIPSMLRRRALKKAETWFTERLNGEDGLGGIFPAMVNAHESLILLGYSPDHPWRVQAKK
ALQNLVIEEKNSASCQPCLSPIWDTGLAALALQETEGGHTTAPVIRALDWLKERQILEQSGDWQVQHPNLKGGGWAF
QYNNSYYPDLDDTALVAWSMDQAATPERYGEAIGRACDWLCGMQSRNGGFAAFESDNTHYYLNEIPFADHGALLDP
PTADVTARCIVLLGRLNKPQYAETLQRALDYLRREQEPNGSWFGRWGTNYIYGTWSALTALEQANIDPQEGFIRKAVE
WLKQVQRLDGGWGEDNYSYFDSSLAGRYQESTPVHTAWALLALMAVGEANSEAVKKGIAYLLQIQQEDGLWDHPAF
NAPGFPRVFYLKYHGYDKFFPLWALARYRNHLNRQC
TelSHC P1 variant: SEQ ID NO: 304
MPTSLATAIDPKQLQQAIRASQDFLFSQQYAEGYWWAELESNVTMTAEVILLHKIWGTEQRLPLAKAEQYLRNHQRD
HGGWELFYGDGGDLSTSVEAYMGLRLLGVPETDPALVKARQFILARGGISKTRIFTKLHLALIGCYDWRGIPSLPPWIML
LPEGSPFTIYEMSSGARSSTVPLLIVMDRKPVYGMDPPITLDELYSEGRANVVWELPRQGDWRDVFIGLDRVFKLFETL
NIHPLREQGLKAAEEWVLERQEASGDWGGIIPAMLNSLLALRALDYAVDDPIVQRGMAAVDRFAIETETEYRVQPCVS
PVWDTALVMRAMVDSGVAPDHPALVKAGEWLLSKQILDYGDWHIKNKKGRPGGWAFEFENRFYPDVDDTAVVVM
ALHAVTLPNENLKRRAIERAVAWIASMQCRPGGWAAFDVDNDQDWLNGIPYGDLKAMIDPNTADVTARVLEMVGR
CQLAFDRVALDRALAYLRNEQEPEGCWFGRWGVNYLYGTSGVLTALSLVAPRYDRWRIRRAAEWLMQCQNADGG
WGETCWSYHDPSLKGKGDSTASQTAWAIIGLLAAGDATGDYATEAIERGIAYLLETQRPDGTWHEDYFTGTGFPCHFY
LKYHYYQQHFPLTALGRYARWRNLLAT
ScoSHC P1 variant: SEQ ID NO: 311
MTATTDGSTGASLRPLAASASDTDITIPAAAAGVPEAAARATRRATDFLLAKQDAEGWWKGDLETNVTMDAEDLLLR
QFLGIQDEETTRAAALFIRGEQREDGTWATFYGGPGELSTTIEAYVALRLAGDSPEAPHMARAAEWIRSRGGIASARVF
TRIWLALFGWWKWDDLPELPPELIYFPTWVPLNIYDFGCGARQTIVPLTIVSAKRPVRPAPFPLDELHTDPARPNPPRPL
APVASWDGAFQRIDKALHAYRKVAPRRLRRAAMNSAARWIIERQENDGCWGGIQPPAVYSVIALYLLGYDLEHPVMR
AGLESLDRFAVWREDGARMIEACQSPVWDTCLATIALADAGVPEDHPQLVKASDWMLGEQIVRPGDWSVKRPGLPP
GGWAFEFHNDNYPDIDDTAEVVLALRRVRHHDPERVEKAIGRGVRWNLGMQSKNGAWGAFDVDNTSAFPNRLPFC
DFGEVIDPPSADVTAHVVEMLAVEGLAHDPRTRRGIQWLLDAQETDGSWFGRWGVNYVYGTGSVIPALTAAGLPTS
HPAIRRAVRWLESVQNEDGGWGEDLRSYRYVREWSGRGASTASQTGWALMALLAAGERDSKAVERGVAWLAATQ
REDGSWDEPYFTGTGFPWDFSINYNLYRQVFPLTALGRYVHGEPFAKKPRAADAPAEAAPAEVKGS
ZmoSCH2 P1 variant: SEQ ID NO: 313
MTVSTSSAFHHSPLSDDVEPIIQKATRALLEKQQQDGHWVFELEADATIPAEYILLKHYLGEPEDLEIEAKIGRYLRRIQGE
HGGWSLFYGGDLDLSATVKAYFALKMIGDSPDAPHMLRARNEILARGGAMRANVFTRIQLALFGAMSWEHVPQMP
VELMLMPEWFPVHINKMAYGARTVLVPLLVLQALKPVARNRRGILVDELFVPDVLPTLQESGDPIWRRFFSALDKVLHK
VEPYWPKNMRAKAIHSCVHFVTERLNGEDGLGAIYPAIANSVMMYDALGYPENHPERAIARRAVEKLMVLDGTEDQG
DKEVYCQPCLSPIWDTALVAHAMLEVGGDEAEKSAISALSWLKPQQILDVKGDWAWRRPDLRPGGWAFQYRNDYYP
DVDDTAVVTMAMDRAAKLSDLHDDFEESKARAMEWTIGMQSDNGGWGAFDANNSYTYLNNIPFADHGALLDPPT
VDVSARCVSMMAQAGISITDPKMKAAVDYLLKEQEEDGSWFGRWGVNYIYGTWSALCALNVAALPHDHLAVQKAV
AWLKTIQNEDGGWGENCDSYALDYSGYEPMDSTASQTAWALLGLMAVGEANSEAVTKGINWLAQNQDEEGLWKE
DYYSGGGFPRVFYLRYHGYSKYFPLWALARYRNLKKANQPIVHYGM

TABLE 18
The correspondence between sequence ID numbers of WO2016/170099
and WO2021/110848 and the new sequence ID numbers of
this disclosure is indicated in the table below.

| Source | Original SEQ ID NO | New SEQ ID NO |
|---|---|---|
| WO2016/170099 | 5 | 361 |
| WO2016/170099 | 7 | 362 |
| WO2016/170099 | 9 | 363 |
| WO2016/170099 | 11 | 364 |
| WO2016/170099 | 13 | 365 |
| WO2016/170099 | 15 | 366 |
| WO2016/170099 | 17 | 367 |
| WO2016/170099 | 19 | 368 |
| WO2016/170099 | 23 | 369 |
| WO2016/170099 | 25 | 370 |
| WO2016/170099 | 27 | 371 |
| WO2016/170099 | 29 | 372 |
| WO2016/170099 | 31 | 373 |
| WO2016/170099 | 33 | 374 |
| WO2016/170099 | 35 | 375 |
| WO2016/170099 | 37 | 376 |
| WO2016/170099 | 39 | 377 |
| WO2016/170099 | 171 | 378 |
| WO2021/110848 | 2 | 379 |
| WO2021/110848 | 4 | 380 |
| WO2021/110848 | 5 | 381 |
| WO2021/110848 | 17 | 382 |
| WO2021/110848 | 18 | 383 |

TABLE 19
summary of all sequences

| | | SHC |
|---|---|---|
| | | Name/Accession |
| Amino acid | | No/PCT |
| SEQ ID No. | Mutation (s) | publication |
| SEQ ID No. 1 | None | AacSHC |
| SEQ ID No. 2 | M132R + A224V + I432T + A557T + | SHC65 |
| | R613S | |
| SEQ ID No. 3 | M132R + A224V + I432T | 215G2 |
| SEQ ID No. 4 | W169G | AacSHC P1 |
| SEQ ID No. 5 | W169G + G600M | AacSHCP2 |
| SEQ ID No. 6 | W169G + G600M + A306V | AacSHCP3 |
| SEQ ID No. 7 | M132R + A224V + I432T + A557T + | SHC65P1 |
| | R613S + W169G | |
| SEQ ID No. 8 | M132R + A224V + I432T + A557T + | SHC65P2 |
| | R613S + W169G + G600M | |
| SEQ ID No. 9 | M132R + A224V + I432T + A557T + | SHC65P3 |
| | R613S + W169G + G600M + A306V | |
| SEQ ID No. 10 | M132R + A224V + I432T + W169G | 215G2SHCP1 |
| SEQ ID No. 11 | M132R + A224V + I432T + W169G + | 215G2SHCP2 |
| | G600M | |
| SEQ ID No. 12 | M132R + A224V + I432T + W169G + | 215G2SHCP3 |
| | G600M + A306V | |
| SEQ ID No. 13 | Zymomonas mobilis subsp. mobilis | AAV90172.1 |
| | ZM4 ATCC 31821 | |
| SEQ ID No. 14 | Zymomonas mobilis (ZmoSHC1) | WO2010139719 |
| | ATCC31821 | US20120135477 |
| | PF62207_2 | |
| | Genpept Accession No. AAV90172 | |
| | AAF12829.1 | |
| | AVZ42714.1 | |
| SEQ ID No. 15 | ZmoSHC2 | WO2010139719 |
| | EMBL/Genbank Accession No. | US20120135477 |
| | X80766 | |
| SEQ ID No. 16 | Bradryhizobium japonicum (BjpSHC) | WO2010139719 |
| | PF62207_5 | US20120135477 |
| | ABQ33590. | |
| SEQ ID No. 17 | Burkholderia ambifaria | WO2010139719 |
| | | US20120135477 |
| SEQ ID No. 18 | Burkholderia ambifaria | WO2010139719 |
| | | US20120135477 |
| SEQ ID No. 19 | Bacillus anthracis | WO2010139719 |
| | | US20120135477 |
| SEQ ID No. 20 | Frankia alni | WO2010139719 |
| | | US20120135477 |
| SEQ ID No. 21 | Rhodopseudomonas palustris | WO2010139719 |
| | | US20120135477 |
| SEQ ID No. 22 | Gluconobacter morbifer (WT | WO2018157021 |
| | GmoSHC) | EHH69691.1 |
| SEQ ID No. 23 | Thermosynechococcus elongatus | BAC09861.1 |
| | (TelSHC) | |
| SEQ ID No. 24 | Acetobacter pasteurianus (ApaSHC1) | ASC07046.1 |
| SEQ ID No. 25 | Bacillus megaterium (BmeSHC) | WP_016763969 |
| SEQ ID No. 26 | Streptomyces albolongus SalSHC) | AZN28579 |
| SEQ ID No. 27 | Acetobacter pasteurianus ApaSHCA) | WP_003625617 |
| SEQ ID No. 28 | Burkholderia ambifaria BamSHC1) | AJY25620.1 |
| SEQ ID No. 29 | Burkholderia ambifaria (BamSHC2) | ABI91648.1 |
| SEQ ID No. 30 | Pelobacter carbinolicus (PcaSHC2) | ABA87615.1 |
| | or Syntrophotalea carbinolica DSM | |
| | 2380 | |
| SEQ ID No. 31 | Rhodopseudomonas palustris | ABJ08391.1 |
| | (RpaSHC1) | |
| SEQ ID No. 32 | Streptomyces coelicolor (ScoSHC) | CAB39697.1 |
| SEQ ID No. 33 | Syntrophobacter fumaroxidans | ABK17672.1 |
| | (SfuSHC) | |
| SEQ ID No. 34 | Teredinibacter turnerae (TtuSHC) | ACR13362.1 |
| SEQ ID No. 35 | SEQ ID NO: 35 is the nucleotide | |
| | sequence encoding the wild-type | |
| | AacSHC. | |
| SEQ ID No. 36 | SEQ ID NO: 36 is the nucleotide | |
| | sequence encoding the polypeptide of | |
| | SEQ ID NO: 2 (SHC#65 variant). | |
| SEQ ID No. 37 | SEQ ID NO: 37 is the nucleotide | |
| | sequence encoding the 215G2 variant | |
| | (SEQ ID NO: 3). | |
| SEQ ID No. 38 | SEQ ID NO: 38 is the nucleotide | |
| | sequence encoding the AacSHC P1 | |
| | represnted by SEQ ID NO: 4. | |
| SEQ ID No. 39 | SEQ ID NO: 39 is the nucleotide | |
| | sequence encoding the AacSHC P2 | |
| | represented by SEQ ID NO: 5. | |
| SEQ ID No. 40 | SEQ ID NO: 40 is the nucleotide | |
| | sequence encoding the AacSHC P3 | |
| | represented by SEQ ID NO: 6. | |
| SEQ ID No. 41 | SEQ ID NO: 41 is the nucleotide | |
| | sequence encoding the SHC#65 P1 | |
| | variant represented by SEQ ID NO: 7. | |
| SEQ ID No. 42 | SEQ ID NO: 42 is the nucleotide | |
| | sequence encoding the SHC#65 P2 | |
| | variant represented by SEQ ID NO: 8. | |
| SEQ ID No. 43 | SEQ ID NO: 43 is the nucleotide | |
| | sequence encoding the SHC#65 P3 | |
| | variant represented by SEQ ID NO: 9. | |
| SEQ ID No. 44 | SEQ ID NO: 44 is the nucleotide | |
| | sequence encoding the 215G2 P1 | |
| | variant represented by SEQ ID NO: 10 | |
| SEQ ID No. 45 | SEQ ID NO: 45 is the nucleotide | |
| | sequence encoding the 215G2 P2 | |
| | variant represented by SEQ ID NO: 11 | |
| SEQ ID No. 46 | SEQ ID NO: 46 is the nucleotide | |
| | sequence encoding the 215G2 P3 | |
| | variant represented by SEQ ID NO: 12 | |
| SEQ ID No. 47 | M132R + A224V + I432T + Y81H + | SHC66 |
| | A557T + R613S | |
| SEQ ID No. 48 | M132R + A224V + I432T + Y81H + | SHC110B8 |
| | H431L + A557T | |
| SEQ ID No. 49 | M132R + A224V + I432T + T90A + | SHC90C7 |
| | R613S | |
| SEQ ID No. 50 | M132R + A224V + I432T + A172T + | SHC115A7 |
| | M277K | |
| SEQ ID No. 51 | M132R + A224V + I432T + L37Q | |
| SEQ ID No. 52 | M132R + A224V + I432T + V174I | |
| SEQ ID No. 53 | M132R + A224V + I432T + V174I + | |
| | F601Y | |
| SEQ ID No. 54 | M132R + A224V + I432T + L37Q + | |
| | V1741 + F601Y | |
| SEQ ID No. 55 | Acetobacter pasteurianus | |
| | IFO 3283-01 | |
| | BAH99456.1 squalenehopene | |
| | cyclase[Acetobacter pasteurianus | |
| | IFO3283-01] | |
| SEQ ID No. 56 | Acidithiobacillus caldus | EET25937.1 |
| SEQ ID No. 57 | Acidithiobacillus ferrooxidans | ACH84004.1 |
| SEQ ID No. 58 | Acidothermus cellulolyticus | ABK53469.1 |
| SEQ ID No. 59 | Adiantum capillus-veneris | BAF93209.1 |
| SEQ ID No. 60 | Histoplasma capsulatum H143 | EER40510.1 |
| SEQ ID No. 61 | Histoplasma capsulatum G186AR | EEH02950.1 |
| SEQ ID No. 62 | Alicyclobacillus acidocaldarius LAA1 | EED08231.1 |
| SEQ ID No. 63 | squalene-hopene cyclase-partial | |
| | [Alicyclobacillus acidocaldarius] | |
| SEQ ID No. 64 | Alicyclobacillus acidoterrestris | CAA61950.1 |
| SEQ ID No. 65 | Anabaena variabilis | ABA24268.1 |
| SEQ ID No. 66 | Anaeromyxobacter sp. | ABS28257.1 |
| SEQ ID No. 67 | Aspergillus clavatus | EAW07713.1 |
| SEQ ID No. 68 | Aspergillus flavus | EED48353.1 |
| SEQ ID No. 69 | Aspergillus fumigatus | EDP50814.1 |
| SEQ ID No. 70 | Aspergillus fumigatus | EAL84865.1 |
| SEQ ID No. 71 | Aspergillus fumigatus | EAL86291.2 |
| SEQ ID No. 72 | Aspergillus niger | CAK43501.1 |
| SEQ ID NO 73 | Aspergillus niger | |
| SEQ ID No. 74 | Aspergillus oryzae | BAE63941.1 |
| SEQ ID No. 75 | Bacillus velezensis FZB42 | ABS74269.1 |
| SEQ ID No. 76 | Bacillus wiedmannii | EEK66523.1 |
| SEQ ID No. 77 | Bacillus cereus G9241 | EAL12758.1 |
| SEQ ID No. 78 | Bacillus cereus Rock3-42 | EEL44583.1 |
| SEQ ID No. 79 | Bacillus cereus m1293 | EEK43841.1 |
| SEQ ID No. 80 | Bacillus cereus | EEK82938.1 |
| SEQ ID No. 81 | Bacillus cereus | EEK99528.1 |
| SEQ ID No. 82 | Bacillus cereus | EEK77935.1 |
| SEQ ID No. 83 | Bacillus cereus | EEL81079.1 |
| SEQ ID No. 84 | Bacillus cereus m1550 | EEK88307.1 |
| SEQ ID No. 85 | Bacillus cereus F65185 | EEL63943.1 |
| SEQ ID No. 86 | Bacillus cereus Rock4-18 | EEL59884.1 |
| SEQ ID | Bacillus cereus | EEL69857.1 |
| No. 87Molybdopterin | | |
| converting factor, | | |
| subunit 1 [Bacillus | | |
| cereus Rock4-18] | | |
| EEL59884.2 SEQ | | |
| ID No. 88 | | |
| SEQ ID No. 89 | Bacillus cereus AH1273 | EEL92663.1 |
| SEQ ID No. 90 | Bacillus cereus Rock3-44 | EEL49968.1 |
| SEQ ID No. 91 | Bacillus cereus E33L | AAU16998.1 |
| SEQ ID No. 92 | Bacillus cereus ATCC 10987 | AAS42477.1 |
| SEQ ID No. 93 | Bacillus cereus G9842 | ACK95843.1 |
| SEQ ID No. 94 | Bacillus coahuilensis | ZP_03226483.1 |
| SEQ ID No. 95 | Bacillus cytotoxicus | ABS22481.1 |
| SEQ ID No. 96 | Bacillus licheniformis DSM 13 | AAU23777.1 |
| SEQ ID No. 97 | Bacillus mycoides DSM 2048 | EEL98438.1 |
| SEQ ID No. 98 | Bacillus pseudomycoides | EEM04821.1 |
| SEQ ID No. 99 | Bacillus pseudomycoides DSM 12442 | EEM16144.1 |
| SEQ ID No. 100 | Bacillus pumilus SAFR-032 | ABV62529.1 |
| SEQ ID No. 101 | Bacillus pumilus ATCC 7061 | EDW21137.1 |
| 102 | Bacillus sp. NRRL B-14911 | EAR64404.1 |
| 103 | Bacillus sp. SG-1 | EDL66148.1 |
| 104 | Bacillus subtilis | Q796C3.1 |
| 105 | Bacillus subtilis | AAB84441.1 |
| 106 | Bacillus thuringiensis str. Al Hakam | ABK86448.1 |
| 107 | Bacillus thuringiensis serovar | EEM21409.1 |
| | tochigiensis BGSC 4Y1 | |
| 108 | Bacillus thuringiensis serovar | EEM82653.1 |
| | huazhongensis BGSC 4BD1 | |
| 109 | Bacillus thuringiensis serovar kurstaki | EEM52372.1 |
| | str. T03a001 | |
| 110 | Bacillus thuringiensis Bt407 | EEM27851.1 |
| 111 | Bacillus thuringiensis serovar sotto | EEM40716.1 |
| | str. T04001 | |
| 112 | Bacillus thuringiensis serovar pakistani | EEM46814.1 |
| | str. T13001 | |
| 113 | Bacillus thuringiensis IBL 200 | EEM94969.1 |
| 114 | Bacillus mycoides KBAB4 | ABY44436.1 |
| 115 | Pedosphaera parvula Ellin514 | EEF59508.1 |
| 116 | Beijerinckia indica subsp. indica ATCC | ACB96717.1 |
| | 9039 | |
| 117 | Blastopirellula marina DSM 3645 | EAQ81955.1 |
| 118 | Blastopirellula marina DSM 3645 | EAQ78122.1 |
| 119 | Bradyrhizobium japonicum | CAA60250.1 |
| 120 | Acetobacter pasteurianus IFO 3283- | BAH98349.1 |
| | 01 | |
| 121 | Bradyrhizobium sp. ORS 278 | CAL79893.1 |
| 122 | Brevibacillus brevis NBRC 100599 | BAH44778.1 |
| 123 | Burkholderia ambifaria IOP40-10 | EDT05097.1 |
| 124 | Burkholderia ambifaria MEX-5 | EDT37649.1 |
| 125 | Burkholderia ambifaria MC40-6 | ACB68303.1 |
| 126 | Burkholderia ambifaria MEX-5 | EDT42454.1 |
| 127 | Burkholderia cenocepacia PC184 | EAY66961.1 |
| 128 | Burkholderia cenocepacia | ACA95661.1 |
| 129 | Burkholderia cenocepacia J2315 | CAR57099.1 |
| 130 | Burkholderia cenocepacia J2315 | CAR56694.1 |
| 131 | Burkholderia dolosa AU0158 | EAY71311.1 |
| 132 | Burkholderia glumae BGR1 | ACR32572.1 |
| 133 | Burkholderia glumae BGR1 | ACR30752. |
| 134 | Burkholderia graminis C4D1M | EDT12320.1 |
| 135 | Burkholderia mallei SAVP1 | ABM48844.1 |
| 136 | Burkholderia multivorans ATCC 17616 | ABX19650.1 |
| 137 | Burkholderia multivorans ATCC 17616 | ABX16859.1 |
| 138 | Burkholderia phymatum | ACC73258.1 |
| 139 | Burkholderia phytofirmans PsJN | ACD21317.1 |
| 140 | Burkholderia pseudomallei | EEC32728.1 |
| 141 | Burkholderia sp. H160 | EEA03553.1 |
| 142 | Burkholderia lata | ABB06563.1 |
| 143 | Burkholderia lata | ABB10136.1 |
| 144 | Burkholderia sp. CCGE1002 | EFA54357.1 |
| 145 | Burkholderia xenovorans LB400 | ABE35912.1 |
| 146 | Candidatus Koribacter versatilis | ABF40741.1 |
| | Ellin345 | |
| 147 | Candidatus Kuenenia stuttgartiensis | CAJ71215.1 |
| 148 | Candidatus Solibacter usitatus | ABJ82180.1 |
| | Ellin6076 | |
| 149 | Candidatus Solibacter usitatus | ABJ82254.1 |
| | Ellin6076 | |
| 150 | Catenulispora acidiphila DSM 44928 | ACU75510.1 |
| 151 | Chthoniobacter flavus Ellin428 | EDY15838.1 |
| 152 | Chthoniobacter flavus Ellin428 | EDY22035.1 |
| 153 | Crocosphaera watsonii WH 8501 | EAM53094.1 |
| 154 | Cupriavidus taiwanensis LMG 19424 | CAQ72562.1 |
| 155 | Crocosphaera subtropica ATCC | ACB53858.1 |
| | 51142 | |
| 156 | Gloeothece citriformis PCC 7424 | ACK71719.1 |
| 157 | Cyanothece sp. PCC 8802 | EDY02410.1 |
| 158 | Rippkaea orientalis PCC 8801 | ACK66841.1 |
| 159 | Cyanothece sp. PCC 7822 | EDX97382.1 |
| 160 | Crocosphaera chwakensis | EAZ91809.1 |
| 161 | Cyanothece sp. PCC 7425 | ACL45896.1 |
| 162 | Rippkaea orientalis PCC 8802 | ACV02092.1 |
| 163 | Desulfovibrio salexigens DSM 2638 | EEC62384.1 |
| 164 | Dryopteris crassirhizoma | BAG68223.1 |
| 165 | Frankia alni ACN14a | CAJ61140.1 |
| 166 | Frankia alni ACN14a | CAJ60090.1 |
| 167 | Frankia casuarinae | ABD10207.1 |
| 168 | Frankia sp. | ABW15063.1 |
| 169 | Frankia sp. | ABW14125.1 |
| 170 | Frankia sp. Eul1c | EFA59873.1 |
| 171 | Frankia sp. Eul1c | EFA59089.1 |
| 172 | Geobacillus sp. | EED61885.1 |
| 173 | Geobacillus thermodenitrificans | EDY05760.1 |
| 174 | Geobacillus sp. Y412MC52 | EEN95021.1 |
| 175 | Geobacillus sp. Y412MC61 | ACX79399.1 |
| 176 | Geobacter bemidjiensis | ACH40355.1 |
| 177 | Geobacter lovleyi | ACD95949.1 |
| 178 | Geobacter metallireducens | ABB30662.1 |
| 179 | Geobacter metallireducens | ABB33038.1 |
| 180 | Geotalea daltonii FRC-32 | ACM21577.1 |
| 181 | Geobacter sp. M21 | EDV72707.1 |
| 182 | Geotalea daltonii FRC-32. | ACM22003.1 |
| 183 | Geobacter sp. M18 | EET34621.1 |
| 184 | Geobacter sp. M21 | ACT16952.1 |
| 185 | Geobacter sulfurreducens | AAR36453.1 |
| 186 | Geobacter sulfurreducens | AAR34018.1 |
| 187 | Geotalea uraniireducens Rf4 | ABQ25226.1 |
| 188 | Gloeobacter violaceus PCC 7421 | BAC91998.1 |
| 189 | Gluconacetobacter diazotrophicus | ACI51585.1 |
| 190 | Gluconacetobacter diazotrophicus | CAP55563.1 |
| 191 | Gluconobacter oxydans | AAW61994.1 |
| 192 | Granulibacter bethesdensis | ABI63005.1 |
| 193 | Hyphomicrobium denitrificans ATCC | EET65847.1 |
| | 51888 | |
| 194 | Leptospirillum ferrodiazotrophum | EES53667.1 |
| 195 | Leptospirillum rubarum. | EAY57382 |
| 196 | Leptospirillum sp. | EDZ38599.1 |
| 197 | Magnetospirillum magnetotacticum | ZP_00052068.2 |
| 198 | Methylacidiphilum infernorum | ACD82457.1 |
| 199 | Methylorubrum extorquens CM4 | ACK83067.1 |
| 200 | Methylorubrum extorquens | ACK86232.1 |
| 201 | Methylobacterium extorquens | CAX24364.1 |
| 202 | Methylobacterium nodulans | ACL61886.1 |
| 203 | Methylobacterium populi | ACB79998.1 |
| 204 | Methylobacterium radiotolerans | ACB27373.1 |
| 205 | Methylobacterium sp. | ACA20611.1 |
| 206 | Methylocella silvestris | ACK52150.1 |
| 207 | Methylococcus capsulatus | CAA71098.1 |
| 208 | Neosartorya fischeri NRRL 181 | EAW20752.1 |
| 209 | Nitrobacter hamburgensis | ABE63461.1 |
| 210 | Nitrobacter sp. | EAQ34404.1 |
| 211 | Nitrobacter winogradskyi | ABA05523.1 |
| 212 | Nitrococcus mobilis | EAR22397.1 |
| 213 | Nitrosococcus oceani ATCC 19707 | ABA57818.1 |
| 214 | Nitrosomonas eutropha | ABI59752.1 |
| 215 | Nitrosomonas sp. AL212 | EET32702.1 |
| 216 | Nitrosospira multiformis ATCC 25196 | ABB75845.1 |
| 217 | Nostoc punctiforme PCC 73102 | ACC84529.1 |
| 218 | Nostoc sp. PCC 7120 | BAB72732.1 |
| 219 | Afipia carboxidovorans | ACI93782.1 |
| 220 | Paenibacillus sp. | EDS49994.1 |
| 221 | Paenibacillus sp. JDR-2 | ACS99948.1 |
| 222 | Paenibacillus sp. oral taxon 786 | EES74793.1 |
| 223 | Syntrophotalea carbinolica DSM 2380 | ABA87701.1 |
| 224 | Pelobacter propionicus DSM 2379 | ABK98395.1 |
| 225 | Pelobacter propionicus DSM 2379 | ABK98811.1 |
| 226 | Goniophlebium niponicum | BAI48071.1 |
| 227 | Goniophlebium niponicum | BAI48070.1 |
| 228 | Cupriavidus pinatubonensis JMP134 | AAZ64302.1 |
| 229 | Cupriavidus necator H16 | CAJ96989.1 |
| 230 | Ralstonia metallidurans | ABF11015.1 |
| 231 | Ralstonia metallidurans | ABF11268.1 |
| 232 | Rhizobium sp. | P55348.1 |
| 233 | Rhodopirellula baltica | CAD74517.1 |
| 234 | Rhodopseudomonas palustris | ACF02757.1 |
| 235 | Rhodopseudomonas palustris | CAA71101.1 |
| 236 | Rhodopseudomonas palustris | ABD06434.1 |
| 237 | Rhodopseudomonas palustris | ABD87279.1 |
| 238 | Rhodospirillum rubrum ATCC 11170 | ABC20867.1 |
| 239 | Rubrobacter xylanophilus DSM 9941 | ABG05671.1 |
| 240 | Saccharomonospora viridis DSM | ACU97316.1 |
| | 43017 | |
| 241 | Saccharopolyspora erythraea NRRL | CAM03596.1 |
| | 2338 | |
| 242 | Schizosaccharomyces japonicus | EEB08219.1 |
| 243 | Sphaerobacter thermophilus DSM | ACZ39437.1 |
| | 20745 | |
| 244 | Streptomyces albus | ZP_04706071.1 |
| 245 | Streptomyces avermitilis NBRC 14893 | BAC69361.1 |
| 246 | Acaryochloris marina | ABW29816.1 |
| 247 | Streptomyces roseosporus NRRL | ZP_04697579.1 |
| | 15998 | |
| 248 | Streptomyces flavogriseus ATCC | EEW70811.1 |
| | 33331 | |
| 249 | Streptomyces ghanaensis ATCC | ZP_04684415.1 |
| | 14672 | |
| 250 | Streptomyces griseoflavus | ZP_05537325.1 |
| 251 | Streptomyces griseus ATCC 53653 | BAG17791.1 |
| 252 | Streptomyces lividans | ZP_05522220.1 |
| 253 | Streptomyces peucetius subsp. | ACA52082.1 |
| | caesius ATCC 27952 | |
| 254 | Streptomyces pristinaespiralis ATCC | EDY61772.1 |
| | 25486 | |
| 255 | Streptomyces scabiei | CBG68454.1 |
| 256 | Streptomyces sp. | EDX25760.1 |
| 257 | Streptomyces sp. | EDY46371.1 |
| 258 | Streptomyces sp. AA4 | ZP_05479203.1 |
| 259 | Streptomyces sp. | ZP_05510126.1 |
| 260 | Streptomyces sp. Mg1 | ZP_05001249.1 |
| 261 | Streptomyces sp. SPB74 | ZP_04995097.1 |
| 262 | Streptomyces sp. SPB78 | ZP_05490781.1 |
| 263 | Streptomyces sviceus ATCC 29083 | EDY55942.1 |
| 264 | Streptomyces viridochromogenes | ZP_05535608.1 |
| | DSM 40736 | |
| 265 | Synechococcus sp. PCC 7335 | EDX84551.1 |
| 266 | Synechococcus sp. PCC 7335 | ZP_05035816.1 |
| 267 | Synechocystis sp. PCC 6803 | BAA17978.1 |
| 268 | Trichodesmium erythraeum | ABG50159.1 |
| 269 | Verrucomicrobium spinosum DSM | ZP_02925563.1 |
| | 4136 | |
| 270 | Acidiphilium cryptum | ABQ30890.1 |
| 271 | Acetobacter fabarum | PAK78064.1 |
| 272 | Acetobacter orleanensis JCM 7639 | GAN69910.1 |
| 272 | Acetobacter orleanensis JCM 7639 | GAN69910.1 |
| 273 | Acetobacter pasteurianus | WP_012812952.1 |
| 274 | Acidothermus cellulolyticus | WP_011720532.1 |
| 275 | Alicyclobacillus acidocaldarius | WP_012811690.1 |
| 276 | Bacillus megaterium | WP_013083001.1 |
| 277 | Bacillus subtilis | WP_004399534.1 |
| 278 | Bacillus thuringiensis | AJI35613.1 |
| 279 | Brevibacillus thermoruber | WP_029099368.1 |
| 280 | Chloracidobacterium thermophilum | WP_014100779.1 |
| 281 | Cohnella thermotolerans | WP_027091823.1 |
| 282 | Geobacillus thermodenitrificans | WP_029761705.1 |
| 283 | Geobacillus sp. | WP_031409036.1 |
| 284 | Gluconacetobacter xylinus | CUW48332.1 |
| 285 | Gluconobacter frateurii NBRC 103465 | GAD08844.1 |
| 285 | Gluconobacter frateurii NBRC 103465 | GAD08844.1 |
| 286 | Komagataeibacter nataicola | AQU88860.1 |
| 287 | Komagataeibacter xylinus | AHI26287.1 |
| 288 | Methylacidiphilum fumariolicum | WP_009061034.1 |
| 289 | Methylococcus capsulatus | WP_010960137.1 |
| 290 | Sphaerobacter thermophiles | WP_012872483.1 |
| 291 | Syntrophobacter fumaroxidans | WP_011698842.1 |
| 292 | Teredinibacter turnerae | WP_015819476.1 |
| 293 | Thermoactinomyces sp. Gus2-1 | KFZ40906.1 |
| 294 | Zymomonas mobilis | WP_011241313.1 |
| 295 | Alicyclobacillus acidocaldarius | P33247.4 |
| SEQ ID No. 296 | WT Pseudomonas mendocina | |
| | PmeSHC amino acid sequence | |
| SEQ ID No. 297 | nucleotide sequence of SHC/HAC | |
| | enzyme variant #66 | |
| SEQ ID No. 298 | nucleotide sequence of SHC/HAC | |
| | enzyme variant #110B8 | |
| SEQ ID No. 299 | nucleotide sequence of SHC/HAC | |
| | variant #90C7 | |
| SEQ ID No. 300 | nucleotide sequence of SHC/HAC | |
| | variant #115A7 | |
| SEQ ID No. 301 | amino acid sequence of a GmoSHC | |
| | variant with V45L, Q54E, M184I, | |
| | T326S, F624Y | |
| SEQ ID No. 302 | TelSHC with W172G , P311V, F425Y, | |
| | G609A | |
| SEQ ID No. 303 | ZmoSHC1 with Q221S, W222G, | |
| | A368V, F486Y, G667A | |
| SEQ ID No. 304 | TelSHC with W172G | |
| SEQ ID No. 305 | AacSHC with W169G, G600M, | |
| | M132R, A224V and I432T mutations | |
| SEQ ID No. 306 | AacSHC with W169G, G600M , | |
| | M132R and I432T mutations | |
| SEQ ID No. 307 | EHH69691.1 squalene-hopene | |
| | cyclase [Gluconobacter morbifer | |
| | G707] | |
| SEQ ID No. 308 | EER62728.1 squalene-hopene | EER62728.1 |
| | cyclase [Zymomonas mobilis subsp. | |
| | mobilis ATCC 10988] | |
| SEQ ID No. 309 | AHI26287.1 squalene--hopene | AHI26287.1 |
| | cyclase [Komagataeibacter xylinus | |
| | E25] | |
| SEQ ID No. 310 | Geobacillus vulcani | WP_031409036 |
| | WP_031409036.1 MULTISPECIES: | |
| | squalene--hopene cyclase | |
| | [Geobacillus] | |
| SEQ ID No. 311 | Sco SHC with the W196G mutation | |
| SEQ ID No. 312 | ZmoSHC1 with the W222G mutation | |
| SEQ ID No. 313 | ZmoSHC2 with the W177G mutation | |
| SEQ ID No. 314 | <400> 314 | |
| | Asp Xaa Asp Asp Thr Ala | |
| | 1 5 | |
| | Artificial Sequence (Motif) | |
| SEQ ID No. 315 | W169G in WT AacSHC | Mutated SHC of |
| | | Alicyclobacillus |
| | | acidocaldarius |
| SEQ ID No. 316 | W169A | Mutated SHC of |
| | | Alicyclobacillus |
| | | acidocaldarius |
| SEQ ID No. 317 | W169V | Mutated SHC of |
| | | Alicyclobacillus |
| | | acidocaldarius |
| SEQ ID No. 318 | W169G + G600A | Mutated SHC of |
| | | Alicyclobacillus |
| | | acidocaldarius |
| SEQ ID No. 319 | W169G + G600V | Mutated SHC of |
| | | Alicyclobacillus |
| | | acidocaldarius |
| SEQ ID No. 320 | W169G + G600L | Mutated SHC of |
| | | Alicyclobacillus |
| | | acidocaldarius |
| SEQ ID No. 321 | W169G + G600I | Mutated SHC of |
| | | Alicyclobacillus |
| | | acidocaldarius |
| SEQ ID No. 322 | W169G + G600M | Mutated SHC of |
| | | Alicyclobacillus |
| | | acidocaldarius |
| SEQ ID No. 323 | W169A + G600A | Mutated SHC of |
| | | Alicyclobacillus |
| | | acidocaldarius |
| SEQ ID No. 324 | W169A + G600V | Mutated SHC of |
| | | Alicyclobacillus |
| | | acidocaldarius |
| SEQ ID No. 325 | W169A + G600L | Mutated SHC of |
| | | Alicyclobacillus |
| | | acidocaldarius |
| SEQ ID No. 326 | W169A + G600I | Mutated SHC of |
| | | Alicyclobacillus |
| | | acidocaldarius |
| SEQ ID No. 327 | W169A + G600M | Mutated SHC of |
| | | Alicyclobacillus |
| | | acidocaldarius |
| SEQ ID No. 328 | W169V + G600A | Mutated SHC of |
| | | Alicyclobacillus |
| | | acidocaldarius |
| SEQ ID No. 329 | W169V + G600V | Mutated SHC of |
| | | Alicyclobacillus |
| | | acidocaldarius |
| SEQ ID No. 330 | W169V + G600L | Mutated SHC of |
| | | Alicyclobacillus |
| | | acidocaldarius |
| SEQ ID No. 331 | W169V + G600I | Mutated SHC of |
| | | Alicyclobacillus |
| | | acidocaldarius |
| SEQ ID No. 332 | W169V + G600M | Mutated SHC of |
| | | Alicyclobacillus |
| | | acidocaldarius |
| SEQ ID No. 333 | W169G + G600A + A306V | Mutated SHC of |
| | | Alicyclobacillus |
| | | acidocaldarius |
| SEQ ID No. 334 | W169G + G600V + A306V | Mutated SHC of |
| | | Alicyclobacillus |
| | | acidocaldarius |
| SEQ ID No. 335 | W169G + G600L + A306V | Mutated SHC of |
| | | Alicyclobacillus |
| | | acidocaldarius |
| SEQ ID No. 336 | W169G + G600I + A306V | Mutated SHC of |
| | | Alicyclobacillus |
| | | acidocaldarius |
| SEQ ID No. 337 | W169G + G600M + A306V | Mutated SHC of |
| | | Alicyclobacillus |
| | | acidocaldarius |
| SEQ ID No. 338 | W169A + G600A + A306V | Mutated SHC of |
| | | Alicyclobacillus |
| | | acidocaldarius |
| SEQ ID No. 339 | W169A + G600V + A306V | Mutated SHC of |
| | | Alicyclobacillus |
| | | acidocaldarius |
| SEQ ID No. 340 | W169A + G600L + A306V | Mutated SHC of |
| | | Alicyclobacillus |
| | | acidocaldarius |
| SEQ ID No. 341 | W169A + G600I + A306V | Mutated SHC of |
| | | Alicyclobacillus |
| | | acidocaldarius |
| SEQ ID No. 342 | W169A + G600M + A306V | Mutated SHC of |
| | | Alicyclobacillus |
| | | acidocaldarius |
| SEQ ID No. 343 | W169V + G600A + A306V | Mutated SHC of |
| | | Alicyclobacillus |
| | | acidocaldarius |
| SEQ ID No. 344 | W169V + G600V + A306V | Mutated SHC of |
| | | Alicyclobacillus |
| | | acidocaldarius |
| SEQ ID No. 345 | W169V + G600L + A306V | Mutated SHC of |
| | | Alicyclobacillus |
| | | acidocaldarius |
| SEQ ID No. 346 | W169V + G600I + A306V | Mutated SHC of |
| | | Alicyclobacillus |
| | | acidocaldarius |
| SEQ ID No. 347 | W169V + G600M + A306V | Mutated SHC of |
| | | Alicyclobacillus |
| | | acidocaldarius |
| SEQ ID No. 348 | M132R + A224V + I432T | Mutated SHC of |
| | | Alicyclobacillus |
| | | acidocaldarius |
| SEQ ID No. 349 | Nucleotide sequence for WT AacSHC | Coding DNA for |
| | (SEQ ID No. 1) | mutated SHC of |
| | | Alicyclobacillus |
| | | acidocaldarius |
| SEQ ID No. 350 | W169G + A306V | Mutated SHC of |
| | | Alicyclobacillus |
| | | acidocaldarius |
| SEQ ID No. 351 | W169A + A306V | Mutated SHC of |
| | | Alicyclobacillus |
| | | acidocaldarius |
| SEQ ID No. 352 | W169V + A306V | Mutated SHC of |
| | | Alicyclobacillus |
| | | acidocaldarius |
| SEQ ID No. 353 | ZmoSHC1 P2 | SEQ ID NO: 13 |
| | | with the |
| | | substitutions |
| | | W222G and |
| | | G667M |
| SEQ ID No. 354 | ZmoSHC1 P3 | SEQ ID NO: 13 |
| | | with the |
| | | substitutions |
| | | W222G, A368V |
| | | and G667M |
| SEQ ID No. 355 | ZmoSHC2 P2 | SEQ ID NO: 15 |
| | | with the |
| | | substitutions |
| | | W177G and |
| | | G619M |
| SEQ ID No. 356 | ZmoSHC2 P3 | SEQ ID NO: 15 |
| | | with the |
| | | substitutions |
| | | W177G, A321V |
| | | and G619M |
| SEQ ID No. 357 | ScoSHC P2 | SEQ ID NO: 32 |
| | | with the |
| | | substitutions |
| | | W196G and |
| | | G629M |
| SEQ ID No. 358 | ScoSHC P3 | SEQ ID NO: 32 |
| | | with the |
| | | substitutions |
| | | W196G, A335V |
| | | and G629M |
| SEQ ID No. 359 | TelSHC P2 | SEQ ID NO: 23 |
| | | with the |
| | | substitutions |
| | | W172G and |
| | | G609M |
| SEQ ID No. 360 | TelSHC P3 | SEQ ID NO: 23 |
| | | with the |
| | | substitutions |
| | | W172G, A311V |
| | | and G609M |
| SEQ ID No. 361 | T77A | SEQ ID No. 5 of |
| | Synthetic AacSHC Derivative | WO2016/170099 |
| SEQ ID No. 362 | I92V | SEQ ID No. 7 of |
| | Synthetic AacSHC Derivative | WO2016/170099 |
| SEQ ID No. 363 | F129L | SEQ ID No. 9 of |
| | | WO2016/170099 |
| SEQ ID No. 364 | M132R | SEQ ID No. 11 of |
| | | WO2016/170099 |
| SEQ ID No. 365 | A224V | SEQ ID No. 13 of |
| | | WO2016/170099 |
| SEQ ID No. 366 | I432T | SEQ ID No. 15 of |
| | | WO2016/170099 |
| SEQ ID No. 367 | Q579H | SEQ ID No. 17 of |
| | | WO2016/170099 |
| SEQ ID No. 368 | F601Y | SEQ ID No. 19 of |
| | | WO2016/170099 |
| SEQ ID No. 369 | M132R + I432T | SEQ ID No. 23 of |
| | | WO2016/170099 |
| | | SHC26 |
| SEQ ID No. 370 | F601Y | SEQ ID No. 25 of |
| | | WO2016/170099 |
| | | SHC3 |
| SEQ ID No. 371 | T77A + I92V + F129L | SEQ ID No. 27of |
| | | WO2016/170099 |
| | | 111C8 |
| SEQ ID No. 372 | Q579H + F601Y | SEQ ID No. 29 of |
| | | WO2016/170099 |
| | | 101A10 |
| SEQ ID No. 373 | F129L | SEQ ID No. 31 of |
| | | WO2016/170099 |
| | | SHC10 |
| SEQ ID No. 374 | F129L + F601Y | SEQ ID No. 33 of |
| | | WO2016/170099 |
| | | SHC30 |
| SEQ ID No. 375 | F129L + M132R + I432T | SEQ ID No. 35 of |
| | | WO2016/170099 |
| | | SHC31 |
| SEQ ID No. 376 | M132R + I432T + F601Y | SEQ ID No. 37 of |
| | | WO2016/170099 |
| | | SHC32 |
| SEQ ID No. 377 | F129L + M132R + I432T + F601Y | SEQ ID No. 39 of |
| | | WO2016/170099 |
| | | SHC33 |
| SEQ ID No. 378 | F605W | SEQ ID No. 171 |
| | | of |
| | | WO2016/170099 |
| SEQ ID No. 379 | M132R, A224V, I432T, A557T and | SHC#49 |
| | H431L, | |
| SEQ ID No. 380 | M132R, A224V, I432T, Y81H, A557T | SHC#66 |
| | and R613S | |
| SEQ ID No. 381 | M132R, A224V, I432T, Y81H, H431L | SHC#110B8 |
| | and A557T | |
| SEQ ID No. 382 | M132R, A224V, I432T, T90A and | SHC#90C7 |
| | R613S, | |
| SEQ ID No. 383 | M132R, A224V, I432T, A172T and | SHC#115A7 |
| | M277K | |
| SEQ ID No. 384 | Zymomonas mobilis (ZmoSHC1) | SEQ ID NO: 2 of |
| | ATCC31821 | WO2010139719 |
| | PF62207_2 | US20120135477 |
| | Genpept Accession No. AAV90172 | |
| | AAF12829.1 | |
| | AVZ42714.1 | |
| SEQ ID No. 385 | M132R + A224V + I432T + | |
| | W169G + A306V. | |
| SEQ ID No. 386 | M132R + A224V + I432T + A557T + | |
| | R613S + W169G + A306V. | |

1. A process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant, wherein the SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, or 310 and wherein the SHC/HAC enzyme variant amino acid sequence has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 at positions corresponding to positions W169, A306 and/or G600 of SEQ ID NO: 1, 2, or 3 or at a position in an amino acid sequence of a wild type SHC/HAC enzyme corresponding to W169, A306 and/or G600 of SEQ ID NO: 1, 2, or 3.

2. A process for preparing (−)-Ambrox or a mixture comprising (−)-Ambrox, the process comprising enzymatically converting (3E,7E)-homofarnesol (EEH) or a mixture of isomers of homofarnesol comprising EEH to (−)-Ambrox or a mixture comprising (−)-Ambrox using a SHC/HAC enzyme variant, wherein the SHC/HAC enzyme variant has an amino acid sequence having at least 30.0% identity or similarity to SEQ ID NO: 1, 13, 15, 23, 32, and wherein the SHC/HAC enzyme variant amino acid sequence has amino acid alterations relative to SEQ ID NO: 1 at positions corresponding to positions W169, A306 and/or G600 of SEQ ID NO: 1 or at a position in an amino acid sequence of a wild type SHC/HAC enzyme corresponding to W169, A306 and/or G600 of SEQ ID NO: 1.

3. A process for preparing Ambra oxide or a mixture comprising Ambra oxide, the process comprising enzymatically converting (2,E)-Bishomofarnesol (BisEEH) or a mixture of isomers of bishomofarnesol comprising BisEEH to Ambra oxide or a mixture comprising Ambra oxide using a SHC/HAC enzyme variant, wherein the SHC/HAC enzyme variant has an amino acid sequence having at least 30.0% identity or similarity to SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 and wherein the SHC/HAC enzyme variant amino acid sequence has amino acid alterations relative to SEQ ID NO: 1, 2, 3, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 at positions corresponding to positions W169, A306 and/or G600 of SEQ ID NO: 1, 2, or 3 or at a position in an amino acid sequence of a wild type SHC/HAC enzyme corresponding to W169, A306 and/or G600 of SEQ ID NO:1, 2, or 3.

4. A process for preparing Ambra oxide or a mixture comprising Ambra oxide, the process comprising enzymatically converting (2,E)-Bishomofarnesol (BisEEH) or a mixture of isomers of bishomofarnesol comprising BisEEH to Ambra oxide or a mixture comprising Ambra oxide using a SHC/HAC enzyme variant, wherein the SHC/HAC enzyme variant has an amino acid sequence having at least 30.0% identity or similarity to SEQ ID NO: 1, 13, 15, 23, 32, and wherein the SHC/HAC enzyme variant amino acid sequence has amino acid alterations relative to SEQ ID NO: 1 at positions corresponding to positions W169, A306 and/or G600 of SEQ ID NO: 1 or at a position in an amino acid sequence of a wild type SHC/HAC enzyme corresponding to W169, A306 and/or G600 of SEQ ID NO: 1.

5. A process according to any one of the preceding claims, wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, A or V or a functional equivalent thereof, preferably wherein the W at position 169 is replaced by G,
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V or a functional equivalent thereof and/or
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by A, V, L, I or M or a functional equivalent thereof.

6. A process according to any one of the preceding claims, wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G.

7. A process according to any one of the preceding claims, wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, and
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V.

8. A process according to any one of the preceding claims, wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G, and
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by M.

9. A process according to any one of the preceding claims, wherein:
the W at position 169 of SEQ ID NO: 1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by G,
the A at position 306 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54 361, 362, 363, 364, 365, 366, 367, 368, 369, 370, 371, 372, 373, 374, 375, 376, 377, 378, 379, 380, 381, 382 or 383 is replaced by V, and
the G at position 600 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383 is replaced by M.

10. A process according to any one of the preceding claims, wherein the amino acid at position 168 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 is S or the amino acid at a position in an amino acid sequence of a wild type SHC corresponding to 168 of SEQ ID NO:1, 2, 3, 13, 14, 15, 16, 17, 18, 19, 20, 21, 22, 23, 24, 25, 26, 27, 28, 29, 30, 31, 32, 33, 34, 47, 48, 49, 50, 51, 52, 53, 54, 361, 362, 363, 364, 365, 366, 367, 368, 369, 370, 371, 372, 373, 374, 375, 376, 377, 378, 379, 380, 381, 382 or 383, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309 or 310 is S.

11. A process according to any one of the preceding claims, wherein
the SHC/HAC enzyme variant has an amino acid sequence having at least 70.0% identity to SEQ ID NO: 1, 4, 5, 6 or 350 and has at least one of the following mutations: W169G, A306V and G600M or
the SHC/HAC enzyme variant has an amino acid sequence having at least 70.0% identity to SEQ ID NO: 7, 8, 9 or 386 and has the following mutations: M132R, A224V, 1432T, A557T, R613S, and has at least one of the following mutations: W169G, A306V and G600M or
the SHC/HAC enzyme variant has an amino acid sequence having at least 70.0% identity to SEQ ID NO: 10, 11, 12 or 387 and has the following mutations: M132R, A224V, 1432T and has at least one of the following mutations: W169G, A306V and G600M.

12. A process according to any one of claims 1 to 11, wherein the W at position 169 is replaced by G.

13. A process according to any one of claims 1 to 11, wherein the W at position 169 is replaced by G, and the A at position is replaced by V.

14. A process according to any one of claims 1 to 11, wherein the W at position 169 is replaced by G, and the G at position 600 is replaced by M.

15. A process according to any one of claims 1 to 11, wherein the W at position 169 is replaced by G, the A at position 306 is replaced by V, and the G at position 600 is replaced by M.

16. A process according to any one of the preceding claims, wherein
the SHC/HAC enzyme variant has an amino acid sequence having at least 70.0% identity to SEQ ID NO: 23, 304, 359 or 360 and has at least one of the following mutations: W172G, A311V and G609M (Tel SHC/HAC variant) or
the SHC/HAC enzyme variant has an amino acid sequence having at least 70.0% identity to SEQ ID NO: 32, 311, 357, or 358 and has the following mutations: W196G, A335V and G629M (Sco SHC/HAC variant) or
the SHC/HAC enzyme variant has an amino acid sequence having at least 70.0% identity to SEQ ID NO:13, 312, 353 or 354 and has at least one of the following mutations: W222G, A368V and G667M (Zmo SHC1 variant) or
the SHC/HAC enzyme variant has an amino acid sequence having at least 70.0% identity to SEQ ID NO: 15, 313, 355 or 356 and has at least one of the following mutations: W177G, A321V and G619M (Zmo SHC2 variant).

17. A process according to any one of claims 1 to 16, wherein the SHC/HAC enzyme variant has an amino acid sequence having at least 70.0% identity to SEQ ID NO: 23, 304, 359 or 360 and has W172G mutation.

18. A process according to any one of claims 1 to 16, wherein the SHC/HAC enzyme variant has an amino acid sequence having at least 70.0% identity to SEQ ID NO: 23, 304, 359 or 360 and has W172G and A311V mutations.

19. A process according to any one of claims 1 to 16, wherein the SHC/HAC enzyme variant has an amino acid sequence having at least 70.0% identity to SEQ ID NO: 23, 304, 359 or 360 and has W172G and G609M mutations.

20. A process according to any one of claims 1 to 16, wherein the SHC/HAC enzyme variant has an amino acid sequence having at least 70.0% identity to SEQ ID NO: 23, 304, 359 or 360 and has W172G, A311V and G609M mutations.

21. A process according to any one of claims 1 to 16, wherein the SHC/HAC enzyme variant has an amino acid sequence having at least 70.0% identity to SEQ ID NO: 32, 311, 357, or 358 and has W196G mutation.

22. A process according to any one of claims 1 to 16, wherein the SHC/HAC enzyme variant has an amino acid sequence having at least 70.0% identity to SEQ ID NO: 32, 311, 357, or 358 and has W196G and A335V mutations.

23. A process according to any one of claims 1 to 16, wherein the SHC/HAC enzyme variant has an amino acid sequence having at least 70.0% identity to SEQ ID NO: 32, 311, 357, or 358 and has W196G and G629M mutations.

24. A process according to any one of claims 1 to 16, wherein the SHC/HAC enzyme variant has an amino acid sequence having at least 70.0% identity to SEQ ID NO: 32, 311, 357, or 358 and has W196G, A335V and G629M mutations.

25. A process according to any one of claims 1 to 16, wherein the SHC/HAC enzyme variant has an amino acid sequence having at least 70.0% identity to SEQ ID NO:13, 312, 353 or 354 and has W222G mutation.

26. A process according to any one of claims 1 to 16, wherein the SHC/HAC enzyme variant has an amino acid sequence having at least 70.0% identity to SEQ ID NO:13, 312, 353 or 354 and has W222G and A368V mutations.

27. A process according to any one of claims 1 to 16, wherein the SHC/HAC enzyme variant has an amino acid sequence having at least 70.0% identity to SEQ ID NO:13, 312, 353 or 354 and has W222G and G667M mutations.

28. A process according to any one of claims 1 to 16, wherein the SHC/HAC enzyme variant has an amino acid sequence having at least 70.0% identity to SEQ ID NO:13, 312, 353 or 354 and has W222G, A321V and G667M mutations.

29. A process according to any one of claims 1 to 16, wherein the SHC/HAC enzyme variant has an amino acid sequence having at least 70.0% identity to SEQ ID NO: 15, 313, 355 or 356 and has W177G mutation.

30. A process according to any one of claims 1 to 16, wherein the SHC/HAC enzyme variant has an amino acid sequence having at least 70.0% identity to SEQ ID NO: 15, 313, 355 or 356 and has W177G and A321V mutation.

31. A process according to any one of claims 1 to 16, wherein the SHC/HAC enzyme variant has an amino acid sequence having at least 70.0% identity to SEQ ID NO: 15, 313, 355 or 356 and has W177G and G619M mutation.

32. A process according to any one of claims 1 to 16. wherein the SHC/HAC enzyme variant has an amino acid sequence having at least 70.0% identity to SEQ ID NO: 15, 313, 355 or 356 and has W177G, A321V and G619M mutations.

33. A process according to any one of the preceding claims, wherein the SHC/AHC enzyme variant has an amino acid sequence comprising or consisting of or essentially consisting of SEQ ID NO: 4, 5, 6, 7, 8, 9, 10, 11, 12, 305, 306, 304, 302, 311, 312, 313, 353, 354, 355, 356, 357, 358, 359, 360, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 350, 351,352, 385 or 386.

34. A process according to any one of the preceding claims, wherein, the SHC/HAC enzyme variant disclosed herein exhibits:
an increased substrate specificity for EEH (or for BisEEH) when homofarnesol or bis-homofarnesol substrates are used,
an increased product selectivity for (−)-Ambrox (or Ambra oxide) when homofarnesol or bis-homofarnesol substrates are used,
an increased specificity for a particular isomer of substrates when substrates other than homofarnesol or bis-homofarnesol are used (eg ethyl-homofarnesol, hydroxyfarnesylacetone and the like—see, for example WO2021/110858 and WO2021/209482 respectively)
an increased productivity and/or
an increased degree of conversion of EEH (or BisEEH) as well as an increased conversion rate of EEH (or BisEEH) over the first 3 to 6 hours (or over the first 4, 5, or 6 hours) of the reaction, compared to the reference SHC enzyme (e.g. wild-type SHC such as those represented by SEQ ID NO:1, 13, 14, 15, 16, 17, 18, 19, 20, 21, 22, 23, 24, 25, 26, 27, 28, 29, 30, 31, 32, 33, 34, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 307, 308, 309, 310 or 215G2 AacSHC or SHC #65 or a parent SHC enzyme the variant derives from such as those represented by SEQ ID NO:2, 3, 47-48, 49, 50, 51, 52, 53, 54, 361, 362, 363, 364, 365, 366, 367, 368, 369, 370, 371, 372, 373, 374, 375, 376, 377, 378, 379, 380, 381, 382 or 383.

35. A SHC/HAC enzyme variant as defined in any one of the preceding claims.

36. A process according to any one of the preceding claims except claim 35, wherein the enzymatic conversion takes place at a temperature in the range of about 30° C. to about 50° C., for example from about 40° to about 50° C., and/or at a pH in the range of about 5 to about 6.

37. A process according to any one of the preceding claims except claim 35, wherein the process comprises culturing a recombinant host cell that produces the SHC/HAC enzyme variant.

38. A process according to claim 35, wherein the recombinant host cells comprise a nucleic acid sequence selected from SEQ ID NO: 38, 39, 40, 41, 42, 43, 44, 45 or 46.

39. A process according to any one of the preceding claims except claim 35, wherein the mixture of isomers of homofarnesol comprising EEH is an EE:EZ isomer mixture, preferably wherein the EE:EZ isomer mixture is in a weight ratio of 80:20.

40. A process according to any one of the preceding claims except claim 35, wherein the weight ratio of the recombinant host cell producing the SHC/HAC enzyme variant to EEH or a mixture of isomers of homofarnesol comprising EEH in an EE:EZ isomer mixture in a weight ratio of 80:20 is about 2:1, 1:1 or about 0.5:1 or about 0.1:1.

41. A process according to any one of the preceding claims except claim 35, wherein the weight ratio of the recombinant host cell producing the SHC/HAC enzyme variant to EEH or a mixture of isomers of homofarnesol comprising EEH in an EE:EZ isomer mixture is in the range of 0.1-0.5, preferably 0.4 and the concentration of EEH is in the range of 250 g/l to 650 g/l, preferably 450 g/l.

42. A process according to any one of the preceding claims except claim 35, wherein the weight ratio of the recombinant host cell producing the SHC/HAC enzyme variant to EEH or a mixture of isomers of homofarnesol comprising EEH in an EE:EZ isomer mixture is 1 and the concentration of EEH is 250 g/l.

43. (−)-Ambrox obtained by or obtainable by the process of any preceding claim except claim 35, in a solid form in an amorphous or crystalline form.

44. Use of (−)-Ambrox of claim 43 as part of a fragrance or a cosmetic or a consumer product such as fabric care, toiletry, beauty care, a cleaning product, a detergent product, and/or a soap product.

45. A fragrance or a cosmetic or a consumer product comprising (−)-Ambrox of claim 43.

46. A nucleic acid sequence encoding the SHC/HAC enzyme variant of claim 35.

47. A construct comprising the nucleic acid sequence of claim 46.

48. A recombinant host cell comprising the nucleic acid sequence of claim 46 or the construct of claim 47.
